(19) United States
(12) Patent Application Publication
(10) Pub. No.: US 2012/0115848 A1
(43) Pub. Date: May 10, 2012

(54) Inhibitors of Polo-Like Kinase

(75) Inventors: Robert A. Galemmo, JR., San Francisco, CA (US); Dean Richard Artis, I, Kensington, CA (US); Xiaocong Michael Ye, Palo Alto, CA (US); Danielle L. Aubele, Burlingame, CA (US); Anh P. Truong, Burlingame, CA (US); Simeon Bowers, Oakland, CA (US); Roy K. Hom, San Francisco, CA (US); Yong-Liang Zhu, Fremont, CA (US); R. Jeffrey Neitz, San Francisco, CA (US); Jennifer Sealy, Oakland, CA (US); Marc Adler, Orinda, CA (US); Paul Beroza, Belmont, CA (US); John P. Anderson, San Francisco, CA (US)

(73) Assignee: ELAN PHARMACEUTICALS, INC., South San Francisco, CA (US)

(21) Appl. No.: 13/267,834

(22) Filed: Oct. 6, 2011

Related U.S. Application Data
(60) Provisional application No. 61/404,758, filed on Oct. 8, 2010.
(60) Provisional application No. 61/425,560, filed on Dec. 21, 2010.

(51) Int. Cl. A61K 31/55; C07D 471/22; C07D 487/22; C07D 498/22; A61K 31/519; A61P 35/02; A61K 31/5377; A61P 25/00; A61P 25/16; A61P 25/28; A61P 35/00; A61P 35/04; C07D 487/14; A61K 31/5383

(52) U.S. Cl. 514/214.02; 544/251; 544/247; 544/99; 540/578; 544/115; 544/238; 514/250; 514/229.5; 514/233.2

(57) ABSTRACT

The present invention provides compounds having a structure according to Formula (I):

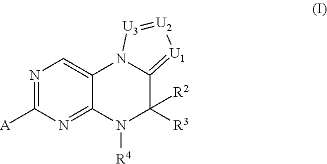

or a salt or solvate thereof, wherein ring A, U¹, U², U³, R², R³ and R⁴ are defined herein. The invention further provides pharmaceutical compositions including the compounds of the invention and methods of making and using the compounds and compositions of the invention, e.g., in the treatment and prevention of various disorders, such as Parkinson's disease.

### CROSS REFERENCE TO RELATED APPLICATIONS

This application claims priority to U.S. Provisional Application No. 61/404,758 entitled “Inhibitors of Polo-Like Kinase” filed Oct. 8, 2010 and U.S. Provisional Application No. 61/425,560 entitled “Inhibitors of Polo-Like Kinase” filed Dec. 21, 2010, each of which is incorporated herein by reference in their entirety.

### BACKGROUND OF THE INVENTION

Lewy body diseases (LBDs) are characterized by degeneration of the dopaminergic system, motor alterations, cognitive impairment, and formation of Lewy bodies (LBs) (see, e.g., McKeith et al, Neurology 1996, 47:1113-1124). LBDs include Parkinson's disease (PD), Diffuse Lewy body disease (DLBD), Lewy body variant of Alzheimer's disease (LBV), combined Parkinson's disease (PD) and Alzheimer's disease (AD), as well as the syndromes identified as multiple system atrophy (MSA). Dementia with Lewy bodies (DLB) is a term coined to reconcile differences in the terminology of LBDs. Disorders with LBs continue to be a common cause for movement disorders and cognitive deterioration in the aging population (see e.g., Galasko et al., Arch. Neurol. 1994, 51:888-895).

In recent years, new hope for understanding the pathogenesis of LBDs has emerged. Several studies suggest that the synaptic protein alpha-synuclein plays a central role in PD pathogenesis. For example, alpha-synuclein accumulates in LBs (see e.g., Spillantini et al., Nature 1997, 388:839-840; Takeda et al., J. Pathol. 1998, 152:367-372; and Wakabayashi et al., Neurosci. Lett. 1997, 239:45-48). Further, mutations in the alpha-synuclein gene co-segregate with rare familial forms of parkinsonism (see e.g., Kruger et al., Nature Gen. 1998, 18:106-8; and Polymeropoulos, et al., Science 1997, 276:2045-2047). In addition, overexpression of alpha-synuclein in transgenic mice (e.g., Masliah et al., Science 2000, 287:1265-1269) and Drosophila (see e.g., Feany et al, Nature 2000, 404:394-398) mimics several pathological aspects of PD.

Many scientists believe that PD is a relatively late development in a systemic synucleinopathy and that “parkinsonism is just the tip of the iceberg” (Langston, Annals of Neurology (2006) 59:591-596). For example, Lewy bodies have been described in sympathetic ganglia and in the myenteric plexus of the gut (Herzog E., Dtch Z Nervenheilk (1928) 107:75-80; Kupsky et al., Neurology (1987) 37:1253-1255). Various disorders have been associated with the presence of Lewy bodies. For example, Lewy bodies have been found in the brain stem of a patient with rapid eye movement sleep behavioral disorder (Uchiyama et al., Neurology (1995) 45:709-712). Olfactory dysfunction has been reported in many PD patients long before the development of parkinsonism. Examination of cardiac tissue from patients with incidental Lewy body disease and typical PD revealed synuclein-positive neuritis in the myocardium (Iwanaga et al., Neurology (1999) 52:1269-1271). There is also evidence that esophageal, lower bowel and bladder dysfunction are early manifestations of PD-related pathology in the peripheral autonomic system (Qualman et al., Gastroenterology (1984) 87:848-856; Castell et al., Neurogasdtroenterol Motil (2001) 13:361-364; Hague et al., Acta Neuropathol (Berl) (1997) 94:192-196). Thus, the fact that accumulation of alpha-synuclein in the brain and other tissues is associated with similar morphological and neurological alterations in species as diverse as humans, mice, and flies suggests that this molecule contributes to the development of PD.

Although the incidence of LBDs continues to increase, creating a serious public health problem, these disorders lack approved treatments.

### SUMMARY OF THE INVENTION

Compounds are provided that are inhibitors of polo-like kinases (PLKs), in particular PLK1 or PLK2, preferably wherein the compound selectively inhibits PLK2 relative to PLK1. PLK2 is a kinase that has been shown to phosphorylate alpha-synuclein, a protein involved in the formation of Lewy bodies Inhibitors of PLK2 are thus useful for the treatment of neurodegenerative diseases, and especially those implicating the formation of Lewy bodies (e.g., Parkinson's disease). Also provided are pharmaceutical compositions comprising inhibitors of PLK2 and methods of utilizing those compositions in the treatment and prevention of various neurodegenerative disorders associated with activation of polo-like kinases, such as Lewy body and Lewy body-type diseases.

Certain PLK inhibitors are known (see, e.g., WO 2008/076392, WO 2009/023269, WO 2010/008454, WO 2010/008459, WO 2010/025073, and U.S. Pat. No. 7,763,629). Typically, those inhibitors are designed to inhibit PLK1, a kinase which is involved in cell proliferation. Consequently those inhibitors are useful for the treatment of various cancers. Thus, compounds described herein that are inhibitors of PLK1 are useful in the treatment of various cancers. PLK inhibitors that are characterized by selectivity for PLK2 over other polo-like kinases, such as PLK1 have not yet been described. Compounds are described herein that are inhibitors of PLK2, preferebly those that are selective relative to PLK1, and are useful in the treatment of neurodegenerative disorders, such as Parkinson's disease and other Lewy body diseases.

In various aspects, compounds are provided having a structure according to Formula (I):

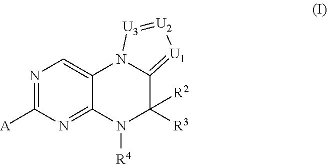

or a salt or solvate thereof, wherein:
A is a ring selected from the group consisting of substituted or unsubstituted aryl, substituted or unsubstituted 5- or 6-membered heterocycloalkyl, and substituted or unsubstituted 5- or 6-membered heteroaryl;
U¹ is N or CR¹, U² is N or CR^{1a} and U³ is N or CR^{1b}, with the proviso that any one or any two of U¹, U² and U³ is N, wherein R¹, R^{1a} and R^{1b}, if present, are independently selected from the group consisting of H, halogen, CN, unsubstituted C₁-C₄ alkyl, and C₁-C₄ haloalkyl;
R² is selected from the group consisting of H, substituted or unsubstituted C₁-C₆ alkyl, substituted or unsubstituted C₂-C₆ alkenyl, substituted or unsubstituted C₂-C₆ alkynyl, substituted or unsubstituted 3- to 6-membered heteroalkyl, substituted or unsubstituted C₃-C₆ cycloalkyl, and substituted or unsubstituted 3- to 6-membered heterocycloalkyl;
R³ is selected from the group consisting of substituted or unsubstituted C₁-C₆ alkyl, substituted or unsubstituted C₂-C₆ alkenyl, substituted or unsubstituted C₂-C₆ alkynyl, substituted or unsubstituted 3- to 6-membered heteroalkyl, substituted or unsubstituted C₃-C₆ cycloalkyl, and substituted or unsubstituted 3- to 6-membered heterocycloalkyl;
or R² and R³, together with the carbon atom to which they are attached, are optionally joined to form a substituted or unsubstituted C₃-C₆ cycloalkyl or a substituted or unsubstituted 3- to 6-membered heterocycloalkyl;
R⁴ is selected from the group consisting of substituted or unsubstituted C₁-C₁₀ alkyl, substituted or unsubstituted C₂-C₁₀ alkenyl, substituted or unsubstituted C₂-C₁₀ alkynyl, substituted or unsubstituted 3- to 10-membered heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, and —NR²⁵R²⁶; or R⁴ and R³, together with the atoms to which they are attached, are optionally joined to form a substituted or unsubstituted 3- to 8-membered heterocyclic ring; or R⁴, R² and R³, together with the atoms to which they are attached, are optionally joined to form a substituted or unsubstituted heterocyclic bicyclic ring system of fused 4- to 8-membered rings; and
R²⁵ and R²⁶ are independently H, substituted or unsubstituted C₃-C₈ cycloalkyl, or substituted or unsubstituted C₁-C₁₀ alkyl.

### DETAILED DESCRIPTION OF THE INVENTION

### Definitions

The definitions and explanations below are for the terms as used throughout this entire document including both the specification and the claims. Throughout the specification and the appended claims, a given formula or name shall encompass all isomers thereof, such as stereoisomers (e.g. diastereomers, enantiomers), geometrical isomers, tautomers, and mixtures thereof where such isomers exist, as well as pharmaceutically acceptable salts and solvates (e.g., hydrates) thereof. In one example, a given formula or name shall encompass all stereoisomers thereof, and pharmaceutically acceptable salts and solvates thereof. In one example, a given formula or name shall encompass all stereoisomers thereof, and pharmaceutically acceptable solvates thereof. In one example, a given formula or name shall encompass all stereoisomers thereof, and pharmaceutically acceptable salts thereof. In one example, a given formula or name shall encompass all pharmaceutically acceptable salts and solvates thereof. In one example, a given formula or name shall encompass all isomers thereof. In one example, a given formula or name shall encompass all stereoisomers thereof. In one example, a given formula or name shall encompass all enantiomers thereof. In one example, a given formula or name shall encompass all diastereomers thereof. In one example, a given formula or name shall encompass all pharmaceutically acceptable salts thereof. In one example, a given formula or name shall encompass all solvates thereof.

Reference to compounds as described herein (e.g. compounds of Formula (I)), or reference to compounds of Formula (I) includes reference to Formula (I) including any sub-generic embodiments thereof, e.g. Formula (I), (Ia), (Ib), (II), (III), (IV), (V), (VI), (VII), (VIII), (VIIIa), (VIIIb), (IX), (IXa), (IXb), (X), (Xa), (Xb), (XI), (XIa), (XIb), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), (XIVf), (XVa), (XVb), (XVc), (XVd), (XVe), (XVf), (XVI), (XVIa), (XVIb), (XVII), (XVId), (XVIe), (XVIf), (XVIg), (XVIIa), (XVIIb), (XVIIc), (XVIId), (XVIIe), or (XVIIf), including all sub-generic embodiments thereof.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Where multiple substituents are indicated as being attached to a structure, those substituents are independently selected. For example “ring A is optionally substituted, e.g., with 1, 2 or 3 R groups” indicates that ring A is substituted with 1, 2 or 3 R groups, wherein the R groups are independently selected (i.e., can be the same or different). It is understood that for any optionally substituted group, any such substitution results in a stable molecule.

Compounds were named using Autonom 2000 4.01.305, which is available from Beilstein Information Systems, Inc, Englewood, Colo.; ChemDraw v.10.0 or ChemDraw Ultra v. 10.0.4, (available from Cambridgesoft at 100 Cambridge Park Drive, Cambridge, Mass. 02140), or ACD Name pro, which is available from Advanced Chemistry Development, Inc., at 110 Yonge Street, 14^{th} floor, Toronto, Ontario, Canada M5c 1T4. Alternatively, the names were generated based on the IUPAC rules or were derived from names originally generated using the aforementioned nomenclature programs. In any instance where there may be any ambiguity between a name given to a compound structure, or if no name is provided for a given structure, the provided structure is intended to clearly define the compound.

The term “alkyl,” by itself or as part of another substituent, means, unless otherwise stated, a straight or branched chain hydrocarbon radical having the number of carbon atoms designated (e.g., C₁-C₁₀ means one to ten carbon atoms). Typically, an alkyl group will have from 1 to 24 carbon atoms (i.e. C₁-C₂₄ alkyl), with those groups having from 1 to 12 carbon atoms (i.e. C₁-C₁₂ alkyl), from 1 to 10 carbon atoms (i.e. C₁-C₁₀ alkyl), from 1 to 8 carbon atoms (i.e. C₁-C₈ alkyl), from 1 to 6 carbon atoms (i.e. C₁-C₆ alkyl) or from 1 to 4 carbon atoms (i.e. C₁-C₄ alkyl) being preferred. A “lower alkyl” group is an alkyl group having from 1 to 4 carbon atoms (i.e. C₁-C₄ alkyl). The term “alkyl” includes di- and multivalent radicals. For example, the term “alkyl” includes “alkylene” wherever appropriate, e.g., when the formula indicates that the alkyl group is divalent or when substituents are joined to form a ring. Examples of alkyl radicals include, but are not limited to methyl, ethyl, n-propyl, iso-propyl, n-butyl, tert-butyl, iso-butyl, sec-butyl, as well as homologs and isomers of, for example, n-pentyl, n-hexyl, n-heptyl and n-octyl.

The term “alkylene” by itself or as part of another substituent means a divalent (diradical) alkyl group, wherein alkyl is defined herein. “Alkylene” is exemplified, but not limited, by —CH₂CH₂CH₂CH₂—. Typically, an “alkylene” group will have from 1 to 24 carbon atoms, with those groups having 10 or fewer carbon atoms (e.g., 1 to 8, 1 to 6, or 1 to 4 carbon atoms) being preferred in the present invention. A “lower alkylene” group is an alkylene group having from 1 to 4 carbon atoms.

The term “alkenyl” by itself or as part of another substituent refers to a straight or branched chain hydrocarbon radical having from 2 to 24 carbon atoms (i.e. C₂-C₂₄ alkenyl) and at least one double bond. A typical alkenyl group has from 2 to 10 carbon atoms (i.e. C₂-C₁₀ alkenyl) and at least one double bond. Preferred alkenyl groups have from 2 to 8 carbon atoms (i.e. C₂-C₈ alkenyl) or from 2 to 6 carbon atoms (i.e. C₂-C₆ alkenyl) and from 1 to 3 double bonds. Exemplary “alkenyl” groups include vinyl, 2-propenyl, 1-but-3-enyl, crotyl, 2-(butadienyl), 2,4-pentadienyl, 3-(1,4-pentadienyl), 2-isopentenyl, 1-pent-3-enyl, 1-hex-5-enyl and the like.

The term “alkynyl” by itself or as part of another substituent refers to a straight or branched chain, unsaturated or polyunsaturated hydrocarbon radical having from 2 to 24 carbon atoms (i.e. C₂-C₂₄ alkynyl) and at least one triple bond. A typical “alkynyl” group has from 2 to 10 carbon atoms (i.e. C₂-C₁₀ alkynyl) and at least one triple bond. Preferred “alkynyl” groups have from 2 to 6 carbon atoms (i.e. C₂-C₆ alkynyl) and at least one triple bond. Exemplary “alkynyl” groups include prop-1-ynyl, prop-2-ynyl (i.e., propargyl), ethynyl and 3-butyryl.

The terms “alkoxy,” “alkylamino” and “alkylthio” (or thioalkoxy) are used in their conventional sense, and refer to substituted or unsubstituted alkyl groups that are attached to the remainder of the molecule via an oxygen atom, an amino group, or a sulfur atom, respectively. “Mono-alkylamino” refers to an amino group substituted with a lower alkyl group and “di-alkylamino” refers to an amino group substituted independently with two lower alkyl groups.

The term “heteroalkyl,” by itself or in combination with another term, means a stable, straight or branched chain hydrocarbon radical consisting of the stated number of carbon atoms (e.g., C₂-C₂₄, C₂-C₁₀, C₂-C₈, or C₂-C₆) and at least one heteroatom selected, e.g., from N, O, S, Si, B and P (preferably N, O and S), wherein the nitrogen, sulfur and phosphorus atoms are optionally oxidized, and the nitrogen atom(s) are optionally quaternized. The heteroatom(s) is/are placed at any interior position of the heteroalkyl group. Examples of heteroalkyl groups include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —CH₂—Si(CH₃)₃, —CH₂—CH═N—OCH₃, and —CH═CH—N(CH₃)—CH₃. Up to two heteroatoms can be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. Typically, a heteroalkyl group will have from 3 to 24 atoms (carbon and heteroatoms, excluding hydrogen) (3- to 24-membered heteroalkyl). In another example, the heteroalkyl group has a total of 3 to 12 atoms (3- to 12-membered heteroalkyl), 3 to 10 atoms (3- to 10-membered heteroalkyl) or from 3 to 8 atoms (3- to 8-membered heteroalkyl). The term “heteroalkyl” includes “heteroalkylene” wherever appropriate, e.g., when the formula indicates that the heteroalkyl group is divalent or when substituents are joined to form a ring.

The term “cycloalkyl” by itself or in combination with other terms, represents a saturated or unsaturated, non-aromatic carbocyclic radical having from 3 to 24 carbon atoms (i.e. C₃-C₂₄ cycloalkyl), with those groups having from 3 to 12 carbon atoms (e.g., C₃-C₁₂ cycloalkyl, C₃-C₁₀ cycloalkyl, C₃-C₈ cycloalkyl or C₃-C₆ cycloalkyl) being preferred. Examples of cycloalkyl include, but are not limited to, cyclopropyl, cyclobutyl, cyclopentyl, cyclohexyl, cycloheptyl, 1-cyclohexenyl, 3-cyclohexenyl, cycloheptyl and the like. The term “cycloalkyl” also includes bridged, polycyclic (e.g., bicyclic) structures, such as norbornyl, adamantyl and bicyclo[2.2.1]heptyl. The “cycloalkyl” group can be fused to at least one (e.g., 1 to 3) other ring selected from aryl (e.g., phenyl), heteroaryl (e.g., pyridyl) and non-aromatic (e.g., carbocyclic or heterocyclic) rings. When the “cycloalkyl” group includes a fused aryl, heteroaryl or heterocyclic ring, then the “cycloalkyl” group is attached to the remainder of the molecule via the carbocyclic ring.

The term “heterocycloalkyl”, “heterocyclic”, “heterocycle”, or “heterocyclyl”, by itself or in combination with other terms, represents a carbocyclic, saturated or unsaturated, non-aromatic ring (e.g., 3- to 10-membered or 3- to 8-membered ring and preferably 4-, 5-, 6- or 7-membered ring) containing at least one and up to 5 heteroatoms selected from, e.g., N, O, S, Si, B and P (preferably N, O and S), wherein the nitrogen, sulfur and phosphorus atoms are optionally oxidized, and the nitrogen atom(s) are optionally quaternized (e.g., from 1 to 4 heteroatoms selected from nitrogen, oxygen and sulfur), or a fused ring system of 4- to 8-membered rings (e.g. bicyclic ring system of fused 4- to 8-membered rings), containing at least one and up to 5 heteroatoms (e.g., from 1 to 5 heteroatoms selected from N, O and S) in stable combinations known to those of skill in the art. Exemplary heterocycloalkyl groups include a fused aryl, heteroaryl or cycloalkyl ring. When the “heterocyclic” group includes a fused aryl, heteroaryl or cycloalkyl ring, then the “heterocyclic” group is attached to the remainder of the molecule via a heterocycle. A heteroatom can occupy the position at which the heterocycle is attached to the remainder of the molecule. Exemplary heterocycloalkyl or heterocyclic groups of the present invention include morpholinyl, thiomorpholinyl, thiomorpholinyl S-oxide, thiomorpholinyl S,S-dioxide, piperazinyl, homopiperazinyl, pyrrolidinyl, pyrrolinyl, imidazolidinyl, tetrahydropyranyl, piperidinyl, tetrahydrofuranyl, tetrahydrothienyl, piperidinyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazolyl, dihydropyridyl, dihydropyrimidinyl, dihydrofuryl, dihydropyranyl, tetrahydrothienyl S-oxide, tetrahydrothienyl S,S-dioxide, homothiomorpholinyl S-oxide, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

By “aryl” is meant an aromatic monocyclic or polycyclic carbocyclic group having 6 to 14 carbon atoms, or 6 to 10 carbon atoms, preferably phenyl. Exemplary aryl groups include a fused cycloalkyl, heterocycloalkyl or heteroaryl ring (e.g., from 1 to 3 other rings). When the “aryl” group includes a fused cycloalkyl, heterocycloalkyl or heteroaryl group, then the “aryl” group is linked to the remainder of the molecule via an aryl ring (e.g., a phenyl ring). In one example of a fused ring, two of the hydrogen atoms on adjacent carbon atoms of the aryl ring are replaced with a substituent of the formula -T-C(O)—(CRR′)_{q}—U—, wherein T and U are independently —NR—, —O—, —CRR′— or a single bond, and q is an integer from 0 to 3, wherein R and R′ are independently hydrogen or (C₁-C₆)alkyl. In one example of a fused ring, two of the hydrogen atoms on adjacent carbon atoms of the aryl ring are replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer from 1 to 4, wherein R and R′ are independently hydrogen or (C₁-C₆)alkyl. One of the single bonds of the ring so formed can optionally be replaced with a double bond. In one example of a fused ring, two of the hydrogen atoms on adjacent carbon atoms of the aryl ring are replaced with a substituent of the formula —(CRR′)ₛ—X—(CR″R′″)_{d}—, where s and d are independently integers from 0 to 3, and X is —O—, —NR′—, —S—, —S(O)—, —S(O)₂—, or —S(O)₂NR′—, wherein R, R′, R″ and R′″ are independently hydrogen or (C₁-C₆)alkyl. An “optionally substituted aryl” group is optionally substituted with one or more substituents as described herein (e.g., with 1 to 5 independent substituents). Non-limiting examples of aryl groups include phenyl, 1-naphthyl, 2-naphthyl, quinoline, indanyl, indenyl, dihydronaphthyl, fluorenyl, tetralinyl, benzo[d][1,3]dioxolyl or 6,7,8,9-tetrahydro-5H-benzo[a]cycloheptenyl. Preferred “aryl” groups include phenyl, benzo[d][1,3]dioxolyl and naphthyl. Particularly preferred is phenyl.

The term “arylalkyl” is meant to include those radicals in which an substituted or unsubstituted aryl group is attached to a substituted or unsubstituted alkylene group to create the radical -alkylene-aryl, wherein alkylene and aryl are defined herein. Exemplary “arylalkyl” groups include benzyl, phenethyl, and the like.

By “aryloxy” is meant the group —O-aryl, where aryl is substituted or unsubstituted aryl as defined herein. In one example, the aryl portion of the aryloxy group is phenyl or naphthyl, and preferably phenyl.

By “arylthiooxy” is meant the group —S-aryl, where aryl is substituted or unsubstituted aryl as defined herein.

The term “heteroaryl” or “heteroaromatic” refers to a polyunsaturated, 5-, 6- or 7-membered aromatic moiety containing at least one heteroatom (e.g., 1 to 5 heteroatoms, and preferably 1-3 heteroatoms) selected from N, O, S, Si and B (preferably N, O and S), wherein the nitrogen and sulfur atoms are optionally oxidized, and the nitrogen atom(s) are optionally quaternized. The “heteroaryl” group can be a single ring or be fused to other aryl, heteroaryl, cycloalkyl or heterocycloalkyl rings (e.g., from 1 to 3 other rings). In one example of a fused ring, two of the hydrogen atoms on adjacent atoms (e.g. carbon or nitrogen) of the heteroaryl ring are replaced with a substituent of the formula -T-C(O)—(CRR′)_{q}—U—, wherein T and U are independently —NR—, —O—, —CRR′— or a single bond, and q is an integer from 0 to 3, wherein R and R′ are independently hydrogen or (C₁-C₆)alkyl. In one example of a fused ring, two of the hydrogen atoms on adjacent atoms of the heteroaryl ring are replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer from 1 to 4, wherein R and R′ are independently hydrogen or (C₁-C₆)alkyl. One of the single bonds of the ring so formed can optionally be replaced with a double bond. In one example of a fused ring, two of the hydrogen atoms on adjacent atoms of the heteroaryl ring are replaced with a substituent of the formula —(CRR′)ₛ—X—(CR″R′″)_{d}—, where s and d are independently integers from 0 to 3, and X is —O—, —NR′—, —S—, —S(O)—, —S(O)₂—, or —S(O)₂NR′—, wherein R, R′, R″ and R′″ are independently hydrogen or (C₁-C₆)alkyl. When the “heteroaryl” group includes a fused aryl, cycloalkyl or heterocycloalkyl ring, then the “heteroaryl” group is attached to the remainder of the molecule via a heteroaryl ring. A heteroaryl group can be attached to the remainder of the molecule through a carbon- or heteroatom. An “optionally substituted heteroaryl” group is optionally substituted with one or more substituents as described herein (e.g., with 1 to 5 independent substituents). In one example, the heteroaryl group has from 4 to 10 carbon atoms and from 1 to 5 heteroatoms selected from O, S and N. Non-limiting examples of heteroaryl groups include pyridyl, pyrimidinyl, quinolinyl, benzothienyl, indolyl, indolinyl, pryidazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, isothiazolyl, naphthyridinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridyl, benzotetrahydrofuranyl, benzotetrahydrothienyl, purinyl, benzodioxolyl, triazinyl, pteridinyl, benzothiazolyl, imidazopyridyl, imidazothiazolyl, dihydrobenzisoxazinyl, benzisoxazinyl, benzoxazinyl, dihydrobenzisothiazinyl, benzopyranyl, benzothiopyranyl, chromonyl, chromanonyl, pyridyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Preferred heteroaryl groups include imidazolyl, pyrazolyl, thiadiazolyl, triazolyl, isoxazolyl, isothiazolyl, imidazolyl, thiazolyl, oxadiazolyl, and pyridyl. Other exemplary heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, pyridin-4-yl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

The term “heteroarylalkyl” is meant to include those radicals in which a substituted or unsubstituted heteroaryl group is attached to a substituted or unsubstituted alkylene group to create the radical -alkylene-heteroaryl, wherein alkylene and heteroaryl are defined herein. Exemplary “heteroarylalkyl” groups include pyridylmethyl, pyimidinylmethyl and the like.

By “heteroaryloxy” is meant the group —O-heteroaryl, where heteroaryl is substituted or unsubstituted heteroaryl as defined herein.

By “heteroarylthiooxy” is meant the group —S—heteroaryl, where heteroaryl is substituted or unsubstituted heteroaryl as defined herein.

Each of the above terms (e.g., “alkyl”, “alkenyl”, “alkynyl”, “cycloalkyl”, “heteroalkyl”, heterocycloalkyl”, “aryl” and “heteroaryl”) are meant to include both substituted and unsubstituted forms of the indicated radical, unless otherwise indicated. The term “substituted” for each type of radical is explained below. When a compound of the invention includes more than one substituent, then each of the substituents is independently selected.

The term “substituted” in connection with alkyl, alkenyl, alkynyl, and heteroalkyl radicals (including those groups referred to as alkylene, heteroalkylene, and the like) refers to one or more, also 1-5, also 1-3, substituents, wherein each substituent is independently selected from the group consisting of 3- to 10-membered heteroalkyl optionally substituted with one or more, also 1-5, also 1-3, independently selected substituents R^{f}, C₃-C₁₀ cycloalkyl optionally substituted with one or more, also 1-5, also 1-3, independently selected substituents R^{f}, 3- to 10-membered heterocycloalkyl optionally substituted with one or more, also 1-5, also 1-3, independently selected substituents R^{f}, aryl optionally substituted with one or more, also 1-5, also 1-3, independently selected substituents R^{f}, heteroaryl optionally substituted with one or more, also 1-5, also 1-3, independently selected substituents R^{f}, —OR^{a}, —SR^{a}, ═O, ═NR^{a}, ═N—OR^{a}, —NR^{a}R^{b}, -halogen, —SiR^{a}R^{b}R^{c}, —OC(O)R^{a}, —C(O)R^{e}, —C(O)OR^{a}, —C(O)NR^{a}R^{b}, —OC(O)NR^{a}R^{b}, —NR^{c}C(O)R^{e}, —NR^{c}C(O)NR^{a}R^{b}, —NR^{c}C(S)NR^{a}R^{b}, —NR^{c}C(O)OR^{a}, —NR^{c}C(NR^{a}R^{b})═NR^{d}, —S(O)R^{e}, —S(O)₂R^{e}, —S(O)₂NR^{a}R^{b}, —NR^{c}S(O)₂R^{a}, —CN and —NO₂. R^{a}, R^{a}, R^{b}, R^{c}, R^{d} and R^{e} at each occurrence are each independently selected from the group consisting of hydrogen, C₁-C₂₄ alkyl (e.g., C₁-C₁₀ alkyl, C₁-C₆ alkyl, or C₁-C₄ alkyl) optionally substituted with one or more, also 1-5, also 1-3, independently selected substituents R^{f}, C₃-C₁₀ cycloalkyl optionally substituted with one or more, also 1-5, also 1-3, independently selected substituents R^{f}, 3- to 10-membered heteroalkyl optionally substituted with one or more, also 1-5, also 1-3, independently selected substituents R^{f}, 3- to 10-membered heterocycloalkyl optionally substituted with one or more, also 1-5, also 1-3, independently selected substituents R^{f}, aryl optionally substituted with one or more, also 1-5, also 1-3, independently selected substituents R^{f}, heteroaryl optionally substituted with one or more, also 1-5, also 1-3, independently selected substituents R^{f}, arylalkyl, wherein the aryl ring is optionally substituted with one or more, also 1-5, also 1-3, independently selected substituents R^{f}, and heteroarylalkyl, wherein the heteroaryl ring is optionally substituted with one or more, also 1-5, also 1-3, independently selected substituents R^{f}, wherein R^{e} is preferably other than hydrogen. When two of the above R groups (e.g., R^{a} and R^{b}) are attached to the same nitrogen atom, they can be combined with the nitrogen atom to form a 5-, 6-, or 7-membered heterocycloalkyl ring optionally substituted with one or more, also 1-5, also 1-3, independently selected substituents R^{f} or a 5- or 7-membered heteroaryl ring optionally substituted with one or more, also 1-5, also 1-3, independently selected substituents R^{f}. For example, —NR^{a}R^{b} is meant to include pyrrolidinyl, N-alkyl-piperidinyl and morpholinyl. R^{f} at each occurrence is independently selected from the group consisting of —R^{g}, —OR^{g}, —SR^{g}, ═NR^{g}, ═N—OR^{g}, —NHR, —NR^{h}R^{g}, —SiR^{g}R^{g}R^{g}, —OC(O)R^{g}, —C(O)R^{g}, —C(O)OR^{g}, —C(O)NHR^{g}, —C(O)NR^{h}R^{g}, —OC(O)NHR^{g}, —OC(O)NR^{h}R^{g}, —NHC(O)R^{g}, —NR^{g}C(O)R^{g}, —NHC(O)NR^{h}R^{g}, —NHC(O)NHR^{g}, —NR^{g}C(O)NH₂, —NR^{g}C(O)NHR^{g}, —NR^{g}C(O)NR^{h}R^{g}, —NHC(S)NR^{h}R^{g}, —NHC(S)NHR^{g}, —NR^{g}C(S)NH₂, —NR^{g}C(S)NHR^{g}, —NR^{g}C(S)NR^{h}R^{g}, —NR^{g}C(O)OH, —NHC(O)OR^{g}, —NR^{g}C(O)OR^{g}, —NHC(NR^{h}R^{g})=NR^{g}, —NHC(NR^{h}R^{g})=NH, —NHC(NHR^{g})=NR^{g}, —NHC(NHR^{g})=NH, —NHC(NH₂)═NR^{g}, —NR^{g}C(NHR^{g})=NR^{g}, —NR^{g}C(NHR^{g})=NH, —NR^{g}C(NH₂)═NR^{g}, —NR^{g}C(NH₂)═NH, —NR^{g}C(NR^{h}R^{g})=NH, —NR^{g}C(NR^{h}R^{g})=NR^{g}, —S(O)₂R^{g}, —S(O)₂NHR^{g}, —S(O)₂NR^{h}R^{g}, —NHS(O)₂R^{g}, —NR^{g}S(O)₂R^{g}, -halogen, ═O, ═NH, ═N—OH, —C(O)OH, —C(O)NH₂, —S(O)₂NH₂, —OC(O)NH₂, —NHC(O)NH₂, —NHC(S)NH₂, —NHC(O)OH, —NHC(NH₂)═NH, —CN, —NO₂, —OH, and —NH₂, wherein R^{h} and R^{g} at each occurrence are independently C₁-C₄ alkyl optionally substituted with one or more, also 1-5, also 1-3, substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NRⁱR^{j}; or —NR^{h}R^{g} forms a 5-, 6-, or 7-membered heterocycloalkyl optionally substituted with one or more, also 1-3, unsubstituted C₁-C₄ alkyl; wherein —NRⁱR^{j} forms a 5-, 6-, or 7-membered heterocycloalkyl optionally substituted with one or more, also 1-3, unsubstituted C₁-C₄ alkyl.

The term “substituted” in connection with cycloalkyl, and heterocycloalkyl radicals refers to one or more, also 1-5, also 1-3, substituents, wherein each substituent is independently selected from the group consisting of C₁-C₆ alkyl optionally substituted with one or more, also 1-5, also 1-3, independently selected substituents R^{f}, 3- to 10-membered heteroalkyl optionally substituted with one or more, also 1-5, also 1-3, independently selected substituents R^{f}, C₃-C₁₀ cycloalkyl optionally substituted with one or more, also 1-5, also 1-3, independently selected substituents R^{f}, 3- to 10-membered heterocycloalkyl optionally substituted with one or more, also 1-5, also 1-3, independently selected substituents R^{f}, aryl optionally substituted with one or more, also 1-5, also 1-3, independently selected substituents R^{f}, heteroaryl optionally substituted with one or more, also 1-5, also 1-3, independently selected substituents R^{f}, —OR^{a}, —SR^{a}, ═O, ═NR^{a}, ═N—OR^{a}, —NR^{a}R^{b}, -halogen, —SiR^{a}R^{b}R^{c}, —OC(O)R^{a}, —C(O)R^{e}, —C(O)OR^{a}, —C(O)NR^{a}R^{b}, —OC(O)NR^{a}R^{b}, —NR^{c}C(O)R^{e}, —NR^{c}C(O)NR^{a}R^{b}, —NR^{c}C(S)NR^{a}R^{b}, —NR^{c}C(O)OR^{a}, —NR^{c}C(NR^{a}R^{b})═NR^{d}, —S(O)R^{e}, —S(O)₂R^{e}, —S(O)₂NR^{a}R^{b}, —NR^{c}S(O)₂R^{a}, —CN and —NO₂; wherein R^{a}, R^{b}, R^{c}, R^{d}, R^{e}, and R^{f} are as defined above for substitutions of alkyl and the like.

The term “substituted” in connection with aryl and heteroaryl groups, refers to one or more, also 1-5, also 1-3, substituents, wherein each substituent is independently selected from the group consisting of substituted or unsubstituted alkyl (e.g., C₁-C₂₄ alkyl, C₁-C₁₂ alkyl, C₁-C₁₀ alkyl, C₁-C₆ alkyl, or C₁-C₄ alkyl), substituted or unsubstituted cycloalkyl (e.g., C₃-C₁₀ cycloalkyl, or C₃-C₈ cycloalkyl), substituted or unsubstituted alkenyl (e.g., C₂-C₁₀ alkenyl or C₂-C₆ alkenyl), substituted or unsubstituted alkynyl (e.g., C₂-C₁₀ alkynyl or C₂-C₆ alkynyl), substituted or unsubstituted heteroalkyl (e.g., 3- to 10-membered heteroalkyl, or 3- to 8-membered heteroalkyl), substituted or unsubstituted heterocycloalkyl (e.g., 3- to 10-membered heterocycloalkyl or 3- to 8-membered heterocycloalkyl), aryl optionally substituted with one or more, also 1-5, also 1-3, independently selected substituents R^{k}, heteroaryl optionally substituted with one or more, also 1-5, also 1-3, independently selected substituents R^{k}, —OR^{m}, —SR^{m}, ═O, ═NR^{m}, ═N—OR^{m}, —NR^{m}Rⁿ, -halogen, —SiR^{m}RⁿR^{o}, —OC(O)R^{q}, —C(O)R^{q}, —C(O)OR^{m}, —C(O)NR^{m}Rⁿ, —OC(O)NR^{m}Rⁿ, —NR^{o}C(O)R^{q}, —NR^{o}C(O)NR^{m}Rⁿ, —NR^{o}C(S)NR^{m}Rⁿ, —NR^{o}C(O)OR^{m}, —NR^{o}C(NR^{m}Rⁿ)=NR^{p}, —S(O)R^{q}, —S(O)₂R^{q}, —S(O)₂NR^{m}Rⁿ, —NR^{o}S(O)₂R^{m}, —CN, —NO₂, and —N₃, in a number ranging from one to the total number of open valences on the aromatic ring system, wherein R^{m}, Rⁿ, R^{o}, R^{p} and R^{q} each are independently selected from the group consisting of hydrogen, substituted or unsubstituted C₁-C₂₄ alkyl (e.g., C₁-C₁₀ alkyl, C₁-C₆ alkyl or C₁-C₄ alkyl), substituted or unsubstituted C₃-C₁₀ cycloalkyl, substituted or unsubstituted C₂-C₂₄ heteroalkyl (e.g., C₂-C₁₀ heteroalkyl or C₂-C₆ heteroalkyl), substituted or unsubstituted 3- to 10-membered heterocycloalkyl, aryl optionally substituted with one or more, also 1-5, also 1-3, independently selected substituents R^{k}, heteroaryl optionally substituted with one or more, also 1-5, also 1-3, independently selected substituents R^{k}, arylalkyl, wherein the aryl ring is optionally substituted with one or more, also 1-5, also 1-3, independently selected substituents R^{f}, and heteroarylalkyl, wherein the heteroaryl ring is optionally substituted with one or more, also 1-5, also 1-3, independently selected substituents R^{f}, wherein R^{q} is preferably other than hydrogen. When two R groups (e.g., R^{m} and Rⁿ) are attached to the same nitrogen atom, they can be combined with the nitrogen atom to form a 5-, 6-, or 7-membered heterocycloalkyl ring optionally substituted with one or more, also 1-5, also 1-3, independently selected substituents R^{f} or a 5- or 7-membered heteroaryl ring optionally substituted with one or more, also 1-5, also 1-3, independently selected substituents R^{f}. For example, —NR^{m}Rⁿ is meant to include pyrrolidinyl, N-alkyl-piperidinyl and morpholinyl. R^{k} is independently selected from the group consisting of C₁-C₁₀ alkyl optionally substituted with one or more, also 1-5, also 1-3, independently selected substituents R^{f}, C₃-C₈ cycloalkyl optionally substituted with one or more, also 1-5, also 1-3, independently selected substituents R^{f}, C₂-C₆ alkenyl optionally substituted with one or more, also 1-5, also 1-3, independently selected substituents R^{f}, C₂-C₆ alkynyl, optionally substituted with one or more, also 1-5, also 1-3, independently selected substituents R^{f}, 3- to 10-membered heteroalkyl, optionally substituted with one or more, also 1-5, also 1-3, independently selected substituents R^{f}, 3- to 8-membered heterocycloalkyl optionally substituted with one or more, also 1-5, also 1-3, independently selected substituents R^{f}, aryl optionally substituted with one or more, also 1-5, also 1-3, independently selected substituents R^{f}, heteroaryl optionally substituted with one or more, also 1-5, also 1-3, independently selected substituents R^{f}, —OR^{r}, —SR^{r}, ═O, —NR^{r}, ═N—OR^{r}, —NR^{r}R^{s}, -halogen, —SiR^{r}R^{s}R^{t}, —OC(O)R^{v}, —C(O)R^{v}, —C(O)OR^{r}, —C(O)NR^{r}R^{s}, —OC(O)NR^{r}R^{s}, —NR^{t}C(O)R^{v}, —NR^{t}C(O)NR^{r}R^{s}, —NR^{t}C(S)NR^{r}R^{s}, —NR^{t}C(O)OR^{r}, —NR^{t}C(NR^{r}R^{s})═NR^{u}, —S(O)R^{v}, —S(O)₂R^{v}, —S(O)₂NR^{r}R^{s}, —NR^{t}S(O)₂R^{v}, —CN, —NO₂, and —N₃, in a number ranging from one to the total number of open valences on the aromatic ring system, wherein R^{r}, R^{s}, R^{t}, R^{u} and R^{v} at each occurrence are each independently selected from the group consisting of hydrogen, C₁-C₆ alkyl optionally substituted with one or more, also 1-5, also 1-3, independently selected substituents R^{f}, C₃-C₈ cycloalkyl optionally substituted with one or more, also 1-5, also 1-3, independently selected substituents R^{f}, 3- to 6-membered heteroalkyl optionally substituted with one or more, also 1-5, also 1-3, independently selected substituents R^{f}, 3- to 8-membered heterocycloalkyl optionally substituted with one or more, also 1-5, also 1-3, independently selected substituents R^{f}, aryl optionally substituted with one or more, also 1-5, also 1-3, independently selected substituents R^{f}, heteroaryl optionally substituted with one or more, also 1-5, also 1-3, independently selected substituents R^{f}, arylalkyl, wherein the aryl ring is optionally substituted with one or more, also 1-5, also 1-3, independently selected substituents R^{f}, and heteroarylalkyl, wherein the heteroaryl ring is optionally substituted with one or more, also 1-5, also 1-3, independently selected substituents R^{f}, wherein R^{v} is preferably other than hydrogen. When two R groups (e.g., R^{r} and R^{s}) are attached to the same nitrogen atom, they can be combined with the nitrogen atom to form a 5-, 6-, or 7-membered heterocycloalkyl ring optionally substituted with one or more, also 1-5, also 1-3, independently selected substituents R^{f} or a 5- or 7-membered heteroaryl ring optionally substituted with one or more, also 1-5, also 1-3, independently selected substituents R^{f}. R^{f} is as defined above for substitutions of alkyl and the like.

The terms “halo” or “halogen,” by themselves or as part of another substituent, mean at least one of fluorine, chlorine, bromine and iodine.

By “haloalkyl” is meant an alkyl radical, wherein alkyl is as defined above and wherein the only substitution(s) are halogen, i.e. at least one hydrogen atom is replaced by a halogen atom. The term “haloalkyl,” is meant to include monohaloalkyl and polyhaloalkyl. For example, the term “halo(C₁-C₄)alkyl” or “C₁-C₄ haloalkyl” is mean to include, but not limited to, chloromethyl, 1-bromoethyl, fluoromethyl, difluoromethyl, trifluoromethyl, 1,1,1-trifluoroethyl and 4-chlorobutyl, and 3-bromopropyl. Similarly, “haloalkoxy” is meant an alkoxy radical as defined above, wherein the only substitution(s) are halogen, i.e. at least one hydrogen atom of the alkyl chain is replaced by a halogen atom. For example, the term “C₁-C₄ haloalkoxy” is mean to include, but not limited to, fluoromethoxy, difluoromethoxy, trifluoromethoxy, 2,2,2-trifluoroethoxy and the like.

As used herein, the term “acyl” describes the group —C(O)R^{w}, wherein R^{w} is selected from hydrogen, unsubstituted C₁-C₂₄ alkyl (e.g., C₁-C₁₀ alkyl, C₁-C₆ alkyl or C₁-C₄ alkyl), unsubstituted C₂-C₂₄ alkenyl (e.g., C₂-C₁₀ alkenyl or C₂-C₆ alkenyl), unsubstituted C₂-C₂₄ alkynyl (e.g., C₂-C₁₀ alkynyl or C₂-C₆ alkynyl), unsubstituted C₃-C₁₀ cycloalkyl, unsubstituted C₂-C₂₄ heteroalkyl (e.g., C₂-C₁₀ heteroalkyl or C₂-C₆ heteroalkyl), unsubstituted 3- to 10-membered heterocycloalkyl, unsubstituted aryl, unsubstituted heteroaryl, unsubstituted arylalkyl and unsubstituted heteroarylalkyl. R^{w} is preferably other than hydrogen. The term “substituted acyl” describes the group —C(O)R^{x}, wherein R^{x} is selected from substituted C₁-C₂₄ alkyl (e.g., C₁-C₁₀ alkyl, C₁-C₆ alkyl or C₁-C₄ alkyl), substituted C₂-C₂₄ alkenyl (e.g., C₂-C₁₀ alkenyl or C₂-C₆ alkenyl), substituted C₂-C₂₄ alkynyl (e.g., C₂-C₁₀ alkynyl or C₂-C₆ alkynyl), substituted C₃-C₁₀ cycloalkyl, substituted C₂-C₂₄ heteroalkyl (e.g., C₂-C₁₀ heteroalkyl or C₂-C₆ heteroalkyl), substituted 3- to 10-membered heterocycloalkyl, substituted aryl, substituted heteroaryl, substituted arylalkyl and substituted heteroarylalkyl.

As used herein, the term “heteroatom” includes oxygen (O), nitrogen (N), sulfur (S), silicon (Si), boron (B) and phosphorus (P). Preferred heteroatoms are O, S and N.

By “oxo” is meant the group ═O.

The symbol “R” is a general abbreviation that represents a substituent group as described herein. Exemplary substituent groups include alkyl, alkenyl, alkynyl, cycloalkyl, heteroalkyl, aryl, heteroaryl and heterocycloalkyl groups, each as defined herein.

As used herein, the term “aromatic ring” or “non-aromatic ring” is consistent with the definition commonly used in the art. For example, aromatic rings include phenyl and pyridyl. Non-aromatic rings include cyclohexanes.

As used herein, the term “fused ring system” means at least two rings, wherein each ring has at least 2 atoms in common with another ring. “Fused ring systems can include aromatic as well as non aromatic rings. Examples of “fused ring systems” are naphthalenes, indoles, quinolines, chromenes and the like. Likewise, the term “fused ring” refers to a ring that has at least two atoms in common with the ring to which it is fused.

The phrase “therapeutically effective amount” as used herein means that amount of a compound, material, or composition of the present invention, which is effective for producing a desired therapeutic effect, at a reasonable benefit/risk ratio applicable to any medical treatment. For example, a “therapeutically effective amount” is an amount effective to reduce or lessen at least one symptom of the disease or condition being treated or to reduce or delay onset of one or more clinical markers or symptoms associated with the disease or condition, or to modify or reverse the disease process.

The terms “treatment” or “treating” when referring to a disease or condition, means producing a desired therapeutic effect. Exemplary therapeutic effects include delaying onset or reducing at least one symptom associated with the disease, positively affecting (e.g., reducing or delaying onset) of a clinical marker associated with the disease and slowing or reversing disease progression.

The term “pharmaceutically acceptable” refers to those properties and/or substances that are acceptable to a patient (e.g., human patient) from a toxicological and/or safety point of view.

The term “pharmaceutically acceptable salts” means salts of the compounds as described herein, e.g. compounds of Formula (I), which are prepared with relatively nontoxic acids or bases, depending on the particular substituents found on the compounds described herein. When compounds of the present invention contain relatively acidic functionalities (e.g., —COOH group), base addition salts can be obtained by contacting the compound (e.g., neutral form of such compound) with a sufficient amount of the desired base, either neat or in a suitable inert solvent. Examples of pharmaceutically acceptable base addition salts include lithium, sodium, potassium, calcium, ammonium, organic amino, magnesium and aluminum salts and the like. When compounds of the present invention contain relatively basic functionalities (e.g., amines), acid addition salts can be obtained, e.g., by contacting the compound (e.g., neutral form of such compound) with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, diphosphoric, monohydrogenphosphoric, dihydrogenphosphoric, sulfuric, monohydrogensulfuric, hydriodic and the like, as well as the salts derived from relatively nontoxic organic acids like formic, acetic, propionic, isobutyric, malic, maleic, malonic, benzoic, succinic, suberic, fumaric, lactic, mandelic, phthalic, benzenesulfonic, p-tolylsulfonic, citric, tartaric, methanesulfonic, 2-hydroxyethylsulfonic, salicylic, stearic and the like. Also included are salts of amino acids such as arginate and the like, and salts of organic acids like glucuronic or galactunoric acids and the like (see, for example, Berge et al., Journal of Pharmaceutical Science, 1977, 66: 1-19). Certain specific compounds of the present invention contain both basic and acidic functionalities that allow the compounds to be converted into either base or acid addition salts.

The neutral forms of the compounds can be regenerated, for example, by contacting the salt with a base or acid and isolating the parent compound in the conventional manner. The parent form of the compound can differ from the various salt forms in certain physical properties, such as solubility in polar solvents, but otherwise the salts are equivalent to the parent form of the compound for the purposes of the present invention.

When a substituent includes a negatively charged oxygen atom “O⁻”, e.g., in “—COO⁻”, then the formula is meant to optionally include a proton or an organic or inorganic cationic counterion. In one example, the resulting salt form of the compound is pharmaceutically acceptable. Further, when a compound of Formula (I) includes an acidic group, such as a carboxylic acid group, e.g., written as the substituent “—COOH”, “—CO₂H” or “—C(O)₂H”, then the formula is meant to optionally include the corresponding “de-protonated” form of that acidic group, e.g., “—COO⁻”, “—CO₂⁻” or “—C(O)₂⁻”, respectively.

In addition to salt forms, the present invention provides compounds, which are in a prodrug form. Prodrugs of the compounds described herein are those compounds that readily undergo chemical changes under physiological conditions to provide the compounds of the present invention. Non-limiting examples of “pharmaceutically acceptable derivative” or “prodrug” include pharmaceutically acceptable esters, phosphate esters or sulfonate esters thereof as well as other derivatives of a compound of this invention which, upon administration to a recipient, is capable of providing, either directly or indirectly, a compound of this invention. Particularly favored derivatives or prodrugs are those that increase the bioavailability of the compounds of this invention when such compounds are administered to a mammal (e.g., by allowing an orally administered compound to be more readily absorbed into the blood stream) or which enhance delivery of the parent compound to a biological compartment (e.g., the brain or lymphatic system) relative to the parent species.

Prodrugs include a variety of esters (i.e., carboxylic acid ester). Ester groups, which are suitable as prodrug groups are generally known in the art and include benzyloxy, di(C₁-C₆)alkylaminoethyloxy, acetoxymethyl, pivaloyloxymethyl, phthalidoyl, ethoxycarbonyloxyethyl, 5-methyl-2-oxo-1,3-dioxol-4-yl methyl, and (C₁-C₆)alkoxy esers, optionally substituted by N-morpholino and amide-forming groups such as di(C₁-C₆)alkylamino. Preferred ester prodrug groups include C₁-C₆ alkoxy esters. Those skilled in the art will recognize various synthetic methodologies that may be employed to form pharmaceutically acceptable prodrugs of the compounds of Formula (I) (e.g., via esterification of a carboxylic acid group).

In an exemplary embodiment, the prodrug is suitable for treatment/prevention of those diseases and conditions that require the drug molecule to cross the blood brain barrier. In a preferred embodiment, the prodrug enters the brain, where it is converted into the active form of the drug molecule. In another example, a prodrug is used to enable an active drug molecule to reach the inside of the eye after topical application of the prodrug to the eye. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention can exist in multiple crystalline or amorphous forms (“polymorphs”). In general, all physical forms are of use in the methods contemplated by the present invention and are intended to be within the scope of the present invention. “Compound or a pharmaceutically acceptable salt, hydrate, polymorph or solvate of a compound” intends the inclusive meaning of “and/or”, in that materials meeting more than one of the stated criteria are included, e.g., a material that is both a salt and a solvate is encompassed.

The compounds of the present invention can contain unnatural proportions of atomic isotopes at one or more of the atoms that constitute such compounds. For example, the compounds can be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention. Compounds described herein, in which one or more of the hydrogen atoms are replaced with another stable isotope of hydrogen (i.e., deuterium) or a radioactive isotope (i.e., tritium), are part of this invention. For example, alkyl groups generically include isotopic variants of hydrogen and carbon, such that methyl, for example, as an option for a variable in any Formula, includes —CH₃, or analogous structure in which any atoms can include any isotopes thereof, for example methyl includes —CD₃, —¹⁴CH₃, and the like.

### Compositions Including Stereoisomers

Compounds as described herein, e.g. compounds of Formula (I), can exist in particular geometric or stereoisomeric forms. The invention contemplates all such compounds, including cis- and trans-isomers, (−)- and (+)-enantiomers, diastereomers, (D)-isomers, (L)-isomers, the racemic mixtures thereof, and other mixtures thereof, such as enantiomerically or diastereomerically enriched mixtures, as falling within the scope of compounds of Formula (I). Additional asymmetric carbon atoms can be present in a substituent such as an alkyl group. All such isomers, as well as mixtures thereof, are intended to be included in this invention. When the compounds described herein contain olefinic double bonds or other centers of geometric asymmetry, and unless specified otherwise, it is intended that the compounds include both E and Z geometric isomers. Likewise, all tautomeric forms and mixtures of tautomers are included.

Optically active (R)- and (S)-isomers and d and/isomers can be prepared using chiral synthons or chiral reagents, or resolved using conventional techniques. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound. If, for instance, a particular enantiomer of a compound of the present invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

As used herein, the term “chiral”, “enantiomerically enriched” or “diastereomerically enriched” refers to a compound having an enantiomeric excess (ee) or a diastereomeric excess (de) of greater than about 50%, preferably greater than about 70% and more preferably greater than about 90%. In general, higher than about 90% enantiomeric or diastereomeric excess is particularly preferred, e.g., those compositions with greater than about 95%, greater than about 97% and greater than about 99% ee or de.

The terms “enantiomeric excess” and “diastereomeric excess” are used in their conventional sense. Compounds with a single stereocenter are referred to as being present in “enantiomeric excess”, those with at least two stereocenters are referred to as being present in “diastereomeric excess”. The value of ee will be a number from 0 to 100, zero being racemic and 100 being enantiomerically pure. For example, a 90% ee reflects the presence of 95% of one enantiomer and 5% of the other(s) in the material in question.

Hence, in one embodiment, compositions are provided including a first stereoisomer and at least one additional stereoisomer of a compound as described herein, e.g. a compound of Formula (I). The first stereoisomer can be present in a diastereomeric or enantiomeric excess of at least about 80%, preferably at least about 90% and more preferably at least about 95%. In a particularly preferred embodiment, the first stereoisomer is present in a diastereomeric or enantiomeric excess of at least about 96%, at least about 97%, at least about 98%, at least about 99% or at least about 99.5%. In another embodiment, the compound of Formula (I) is enantiomerically or diastereomerically pure (diastereomeric or enantiomeric excess is about 100%). Enantiomeric or diastereomeric excess can be determined relative to exactly one other stereoisomer, or can be determined relative to the sum of at least two other stereoisomers. In an exemplary embodiment, enantiomeric or diastereomeric excess is determined relative to all other detectable stereoisomers, which are present in the mixture. Stereoisomers are detectable if a concentration of such stereoisomer in the analyzed mixture can be determined using common analytical methods, such as chiral HPLC.

The term “PLK1-mediated condition”, “polo-like kinase 1 mediated disorder” or any other variation thereof, as used herein means any disease or other condition in which PLK1 is known to play a role, or a disease state that is associated with elevated activity or expression of PLK1. For example, a “PLK1-mediated condition” may be relieved by inhibiting PLK1 activity. Such conditions include various cancers, including bladder, thyroid, ovarian, pancreatic, breast, endometrial, prostate, colorectal, lung (e.g. non small cell lung cancer), head and neck, gastric, oropharyngeal, and esophageal cancers, glioma, glioblastoma, papillary carcinoma, hepatoma, melanoma, lymphomas (e.g. non-Hodgkins lymphoma, Hodgkin's lymphoma), leukemias (e.g. chronic myeloid leukemia, acute myeloid leukemia), advanced metastatic cancers, and advanced solid tumors.

The term “PLK2-mediated condition”, “polo-like kinase 2 mediated disorder” or any other variation thereof, as used herein means any disease or other condition in which PLK2 is known to play a role, or a disease state that is associated with elevated activity or expression of PLK2. For example, a “PLK2-mediated condition” may be relieved by inhibiting PLK2 activity. Such conditions include certain neurodegenerative diseases, such as dementias with Lewy bodies (DLB) or Lewy body diseases (LBDs), such as Parkinson's disease (PD), diffuse Lewy body disease (DLBD), Lewy body variant of Alzheimer's disease (LBV) and Alzheimer's disease (AD), as well as any syndrome identified as multiple system atrophy (MSA).

The term “neurodegenerative diseases” includes any disease or condition characterized by problems with movements, such as ataxia, and conditions affecting cognitive abilities (e.g., memory) as well as conditions generally related to all types of dementia. “Neurodegenerative diseases” may be associated with impairment or loss of cognitive abilities, potential loss of cognitive abilities and/or impairment or loss of brain cells. Exemplary “neurodegenerative diseases” include Alzheimer's disease, Parkinson's disease, amyotrophic lateral sclerosis (ALS), Down syndrome, dementia, multi-infarct dementia, mild cognitive impairment (MC1), epilepsy, seizures, Huntington's disease, neurodegeneration induced by viral infection (e.g. AIDS, encephalopathies), traumatic brain injuries, as well as ischemia and stroke.

The term “neurological disorder” refers to any undesirable condition of the central or peripheral nervous system of a mammal. The term “neurological disorder” includes neurodegenerative diseases (e.g., Alzheimer's disease, Parkinson's disease and amyotrophic lateral sclerosis), neuropsychiatric diseases (e.g. schizophrenia and anxieties, such as general anxiety disorder). Exemplary neurological disorders include MLS (cerebellar ataxia), Huntington's disease, Down syndrome, multi-infarct dementia, status epilecticus, contusive injuries (e.g. spinal cord injury and head injury), viral infection induced neurodegeneration, (e.g. AIDS, encephalopathies), epilepsy, benign forgetfulness, closed head injury, sleep disorders, depression (e.g., bipolar disorder), dementias, movement disorders, psychoses, alcoholism, post-traumatic stress disorder and the like. “Neurological disorder” also includes any undesirable condition associated with the disorder. For instance, a method of treating a neurodegenerative disorder includes methods of treating loss of memory and/or loss of cognition associated with a neurodegenerative disorder. Such method would also include treating or preventing loss of neuronal function characteristic of neurodegenerative disorder.

“Pain” is an unpleasant sensory and emotional experience. Pain classifications have been based on duration, etiology or pathophysiology, mechanism, intensity, and symptoms. The term “pain” as used herein refers to all categories of pain, including pain that is described in terms of stimulus or nerve response, e.g., somatic pain (normal nerve response to a noxious stimulus) and neuropathic pain (abnormal response of a injured or altered sensory pathway, often without clear noxious input); pain that is categorized temporally, e.g., chronic pain and acute pain; pain that is categorized in terms of its severity, e.g., mild, moderate, or severe; and pain that is a symptom or a result of a disease state or syndrome, e.g., inflammatory pain, cancer pain, AIDS pain, arthropathy, migraine, trigeminal neuralgia, cardiac ischaemia, and diabetic peripheral neuropathic pain (see, e.g., Harrison's Principles of Internal Medicine, pp. 93-98 (Wilson et al., eds., 12th ed. 1991); Williams et al., J. of Med. Chem. 42: 1481-1485 (1999), herein each incorporated by reference in their entirety). “Pain” is also meant to include mixed etiology pain, dual mechanism pain, allodynia, causalgia, central pain, hyperesthesia, hyperpathia, dysesthesia, and hyperalgesia.

### Compositions

Certain 2-aryl- or 2-heteroarylpteridinones (e.g., 2-(imidazo)pteridinones) and certain 7-aryl- or 7-heteroaryl dihydropyrido[4,3-b]pyrazinones, e.g. compounds as described herein within the scope of Formula (I), are potent inhibitors of PLK. In addition those compounds exhibit properties conducive to good CNS exposure. Compared to known PLK inhibitors, compounds as described herein are characterized by one or more of the following properties: (i) reduced affinity for the P-glycoprotein (In one example, the compounds exhibit essentially no binding affinity/are no substrate for the P-glycoprotein);

(ii) relatively low molecular weight;
(iii) reduced number of H-bond donors (In one example, the compounds do not incorporate an H-bond donor group);
(iv) reduced total polar surface area (TPSA);
(v) isoform selectivity favoring PLK2 over PLK1; and
(vi) improved solubility.

Furthermore, certain compounds as described herein are characterized by relatively high brain to plasma ratios and good brain exposure as indicated by in vivo experimental results (see, e.g., Example B). The structure of the current PLK inhibitors provides compounds with good CNS exposure properties and isoform selectivity favoring PLK2 over PLK1.

In various aspects, the invention provides a compound having a structure according to Formula (I):

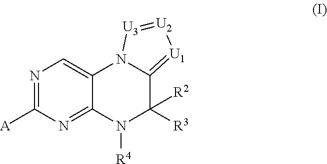

or a salt or solvate thereof, wherein A is a ring selected from the group consisting of substituted or unsubstituted aryl, substituted or unsubstituted 5- or 6-membered heterocycloalkyl and substituted or unsubstituted 5- or 6-membered heteroaryl. In one example, A is substituted or unsubstituted aryl, wherein the aryl is fused to an additional ring, wherein the additional ring is substituted or unsubstituted 5- or 6-membered heterocycloalkyl or substituted or unsubstituted 5- or 6-membered heteroaryl. Exemplary A rings are described herein, below.

In Formula (I), U¹ is N or CR¹, U² is N or CR^{1a} and U³ is N or CR^{1b}, with the proviso that any one or any two of U¹, U² and U³ is N, wherein R¹, R^{1a} and R^{1b}, if present, are independently selected from H, halogen, CN, unsubstituted C₁-C₄ alkyl, and C₁-C₄ haloalkyl.

In Formula (I), R² is selected from the group consisting of H, substituted or unsubstituted C₁-C₆ alkyl, substituted or unsubstituted C₂-C₆ alkenyl, substituted or unsubstituted C₂-C₆ alkynyl, substituted or unsubstituted 3- to 6-membered heteroalkyl, substituted or unsubstituted C₃-C₆ cycloalkyl and substituted or unsubstituted 3- to 6-membered heterocycloalkyl; R³ is selected from the group consisting of substituted or unsubstituted C₁-C₆ alkyl, substituted or unsubstituted C₂-C₆ alkenyl, substituted or unsubstituted C₂-C₆ alkynyl, substituted or unsubstituted 3- to 6-membered heteroalkyl, substituted or unsubstituted C₃-C₆ cycloalkyl and substituted or unsubstituted 3- to 6-membered heterocycloalkyl; or R² and R³, together with the carbon atom to which they are attached, are optionally joined to form a substituted or unsubstituted C₃-C₆ cycloalkyl or a substituted or unsubstituted 3- to 6-membered heterocycloalkyl group; or R⁴ and R³, together with the atoms to which they are attached, are optionally joined to form a substituted or unsubstituted 3- to 8-membered heterocyclic ring; or R⁴, R² and R³, together with the atoms to which they are attached, are optionally joined to form a substituted or unsubstituted heterocyclic bicyclic ring system of fused 4- to 8-membered rings.

In Formula (I), R⁴ is selected from substituted or unsubstituted C₁-C₁₀ alkyl, substituted or unsubstituted C₂-C₁₀ alkenyl, substituted or unsubstituted C₂-C₁₀ alkynyl, substituted or unsubstituted 3- to 10-membered heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, and —NR²⁵R²⁶; or R⁴ and R³, together with the atoms to which they are attached, are optionally joined to form a substituted or unsubstituted 3- to 8-membered heterocyclic ring; or R⁴, R² and R³, together with the atoms to which they are attached, are optionally joined to form a substituted or unsubstituted heterocyclic bicyclic ring system of fused 4- to 8-membered rings; wherein R²⁵ and R²⁶ are independently H, substituted or unsubstituted C₃-C₈ cycloalkyl, or substituted or unsubstituted C₁-C₁₀ alkyl.

In one example in Formula (I), A is linked to the remainder of the compound via a nitrogen atom (N-linked). In one embodiment, the compound of Formula (I) has a structure according to Formula (II):

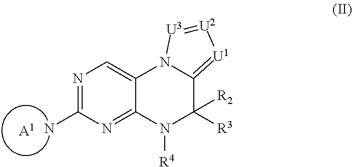

or a salt or solvate thereof, wherein U¹, U², U³, R², R³, and R⁴ are defined as for Formula (I), above, and ring A¹ is substituted or unsubstituted 5- or 6-membered heterocycloalkyl or substituted or unsubstituted 5- or 6-membered heteroaryl.

In one embodiment, the compound of Formula I has a structure according to Formula (III):

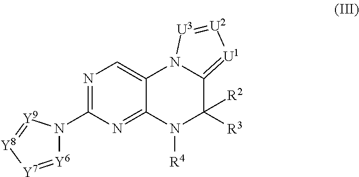

or a salt or solvate thereof, wherein U¹, U², U³, R², R³, and R⁴, are defined as for Formula (I), above. In the above formulae, Y⁶ is N or CR⁶, Y⁷ is N or CR⁷, Y⁸ is N or CR⁸ and Y⁹ is N or CR⁹, wherein at least one of Y⁶, Y⁷, Y⁸ and Y⁹ is other than N. R⁶, R⁷, R⁸ and R⁹ are independently selected from the group consisting of H, substituted or unsubstituted alkyl, substituted or unsubstituted alkenyl, substituted or unsubstituted alkynyl, substituted or unsubstituted heteroalkyl, substituted or unsubstituted cycloalkyl, substituted or unsubstituted heterocycloalkyl, aryl optionally substituted with one or more, also 1-5, also 1-3, independently selected substituents R²⁷, heteroaryl optionally substituted with one or more, also 1-5, also 1-3, independently selected substituents R²⁷, —CN, -halogen, —OR¹², —SR¹², —NR¹²R¹³, —C(O)R¹⁴, —C(O)NR¹²R¹³, —OC(O)NR¹²R¹³, —C(O)OR¹², —NR¹⁵C(O)R¹⁴—NR¹⁵C(O)OR¹², —NR¹⁵C(O)NR¹²R¹³, —NR¹⁵C(S)NR¹²R¹³, —NR¹⁵S(O)₂R¹⁴, —S(O)₂NR¹²R¹³, —S(O)R¹⁴ and —S(O)₂R¹⁴, wherein each occurrence of R¹², R¹³ and R¹⁵ are independently selected from the group consisting of H, substituted or unsubstituted C₁-C₆ alkyl, substituted or unsubstituted 3- to 6-membered heteroalkyl, aryl optionally substituted with one or more, also 1-5, also 1-3, independently selected substituents R²⁷, 5- or 6-membered heteroaryl optionally substituted with one or more, also 1-5, also 1-3, independently selected substituents R²⁷, substituted or unsubstituted C₃-C₈ cycloalkyl and substituted or unsubstituted 3- to 8-membered heterocycloalkyl; each occurrence of R¹⁴ is independently selected from substituted or unsubstituted C₁-C₆ alkyl, substituted or unsubstituted 3- to 6-membered heteroalkyl, aryl optionally substituted with one or more, also 1-5, also 1-3, independently selected substituents R²⁷, 5- or 6-membered heteroaryl optionally substituted with one or more, also 1-5, also 1-3, independently selected substituents R²⁷, substituted or unsubstituted C₃-C₈ cycloalkyl and substituted or unsubstituted 3- to 8-membered heterocycloalkyl; or two of R⁶, R⁷, R⁸ and R⁹ on adjacent ring atoms, together with the ring atoms to which they are attached, are optionally joined to form a 3- to 7-membered ring selected from phenyl optionally substituted with one or more, also 1-5, also 1-3, independently selected substituents R²⁷, heteroaryl optionally substituted with one or more, also 1-5, also 1-3, independently selected substituents R²⁷, cycloalkyl optionally substituted with one or more, also 1-5, also 1-3, independently selected substituents R²⁹, and heterocycloalkyl optionally substituted with one or more, also 1-5, also 1-3, independently selected substituents R²⁹; R²⁷ at each occurrence is selected from the group consisting of C₁-C₁₀ alkyl optionally substituted with one or more, also 1-5, also 1-3, independently selected substituents R²⁸, 3- to 10-membered heteroalkyl optionally substituted with one or more, also 1-5, also 1-3, independently selected substituents R²⁸, C₃-C₈ cycloalkyl optionally substituted with one or more, also 1-5, also 1-3, independently selected substituents R²⁹, 3- to 8-membered heterocycloalkyl optionally substituted with one or more, also 1-5, also 1-3, independently selected substituents R²⁹, aryl optionally substituted with one or more, also 1-5, also 1-3, independently selected substituents R²⁹, heteroaryl optionally substituted with one or more, also 1-5, also 1-3, independently selected substituents R²⁹, —CN, —NO₂, -halogen, —OR³⁰, —SR³⁰, —NR³⁰R³¹, —C(O)R³², —C(O)NR³⁰R³¹, —OC³⁰R³¹, —C(O)OR³⁰, —OC(O)R³², —NR³³C(O)R³², —NR³³C(O)R³⁰, —NR³³C(O)NR³⁰R³¹, —NR³³C(S)NR³⁰R³¹, —NR—S(O)₂NR³⁰R³¹, —S(O)R³² and —S(O)₂R³²; R³⁰, R³¹, R³², and R³³, at each occurrence are independently selected from the group consisting of hydrogen, C₁-C₁₀ alkyl optionally substituted with one or more, also 1-5, also 1-3, independently selected substituents R²⁸, 3- to 12-membered heteroalkyl optionally substituted with one or more, also 1-5, also 1-3, independently selected substituents R²⁸, C₃-C₈ cycloalkyl optionally substituted with one or more, also 1-5, also 1-3, independently selected substituents R²⁹, 3- to 8-membered heterocycloalkyl optionally substituted with one or more, also 1-5, also 1-3, independently selected substituents R²⁹, aryl optionally substituted with one or more, also 1-5, also 1-3, independently selected substituents R²⁹, and heteroaryl optionally substituted with one or more, also 1-5, also 1-3, independently selected substituents R²⁹, provided that R³² is other than hydrogen; R²⁸ at each occurrence is independently selected from the group consisting of aryl optionally substituted with one or more, also 1-5, also 1-3, independently selected substituents R³⁹, heteroaryl optionally substituted with one or more, also 1-5, also 1-3, independently selected substituents R³⁹, —OR³⁴, —SR³⁴, —NHR³⁴, —NR—C(O)R³⁴, —C(O)OR³⁴, —C(O)NHR³⁴, —C(O)NR³⁵R³⁴, —NHC(O)R³⁴, —NR³⁴C(O)R³⁴, —NHC(O)OR³⁴, —NR³⁴C(O)OR³⁴, —NR³⁴C(O)OH, —S(O)₂R³⁴, —S(O)₂NHR³⁴, —S(O)₂NR³⁵R³⁴, —NHS(O)₂R³⁴, —NR³⁴S(O)₂R³⁴, -halogen, —NHC(O)OH, —C(O)OH, —C(O)NH₂, —S(O)₂NH₂, —CN, —NO₂, ═O, —OH, ═NH, and —NH₂; R²⁹ at each occurrence is independently —R²⁸ or —R³⁴; R³⁴ and R³⁵ are independently selected from the group consisting of aryl optionally substituted with one or more, also 1-5, also 1-3, independently selected substituents R³⁹, heteroaryl optionally substituted with one or more, also 1-5, also 1-3, independently selected substituents R³⁹, and C₁-C₄ alkyl optionally substituted with one or more, also 1-5, also 1-3, substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR³⁶R³⁷; or —NR³⁴R³⁵ forms a 5-, 6-, or 7-membered heterocycloalkyl optionally substituted with one or more, also 1-5, also 1-3, unsubstituted C₁-C₄ alkyl; wherein —NR³⁶R³⁷ forms a 5-, 6-, or 7-membered heterocycloalkyl optionally substituted with one or more, also 1-5, also 1-3, unsubstituted C₁-C₄ alkyl; R³⁹ at each occurrence is independently selected from the group consisting of —R⁴⁴, —OR⁴⁴, —SR⁴⁴, —NHR⁴⁴, —NR⁴⁴R⁴⁵, —C(O)R⁴⁴, —C(O)OR⁴⁴, —NHC(O)R⁴⁴, —C(O)NHR⁴⁵, —C(O)NR⁴⁴R⁴⁵, —S(O)₂R⁴⁴, —NHS(O)₂R⁴⁴, —S(O)₂NHR⁴⁵, —S(O)₂NR⁴⁴R⁴⁵, -halogen, —C(O)OH, —C(O)NH₂, —CN, —OH, and —NH₂; R⁴⁴ and R⁴⁵ are independently C₁-C₄ alkyl optionally substituted with one or more, also 1-5, also 1-3, independently selected substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁴⁶R⁴⁷; or —NR⁴⁴R⁴⁵ forms a 5-, 6-, or 7-membered heterocycloalkyl optionally substituted with one or more, also 1-3, unsubstituted C₁-C₄ alkyl; wherein —NR⁴⁶R⁴⁷ forms a 5-, 6-, or 7-membered heterocycloalkyl optionally substituted with one or more, also 1-3, unsubstituted C₁-C₄ alkyl.

In one embodiment, the compound of Formula (I) has a structure according to Formula (IV):

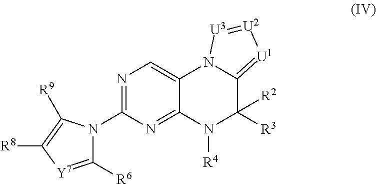

or a salt or solvate thereof, wherein U¹, U², U³, R², R³, and R⁴ are defined as for Formula (I), and Y⁷, R⁶, R⁸ and R⁹ are defined as for Formula (III), above.

In one example in Formula (I), ring A is linked to the remainder of the molecule via a carbon atom (C-linked). In one embodiment the compound of Formula (I) has a structure according to Formula (V):

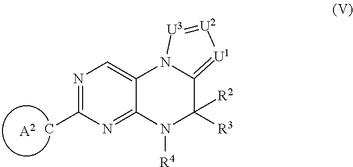

or a salt or solvate thereof, wherein U¹, U², U³, R², R³, and R⁴ are defined as for Formula (I), above, and ring A² is substituted or unsubstituted 5- or 6-membered heterocycloalkyl or substituted or unsubstituted 5- or 6-membered heteroaryl.

In one example in Formula (V), A² is selected from the group consisting of:

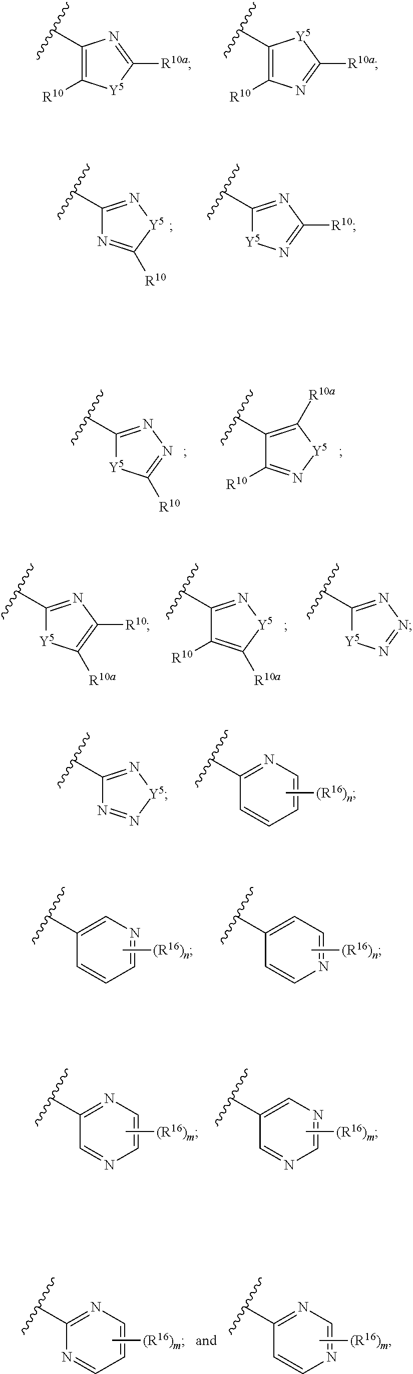

wherein n is an integer selected from 0 to 4 and m is an integer selected from 0 to 3; Y⁵ is O, S or NR¹¹, wherein R¹¹ is selected from the group consisting of H, —C(O)R²², substituted or unsubstituted C₁-C₆-alkyl, substituted or unsubstituted 3- to 6-membered heteroalkyl, aryl optionally substituted with one or more, also 1-5, also 1-3, independently selected substituents R²⁷, 5- or 6-membered heteroaryl optionally substituted with one or more, also 1-5, also 1-3, independently selected substituents R²⁷, substituted or unsubstituted C₃-C₈ cycloalkyl and substituted or unsubstituted 3- to 8-membered heterocycloalkyl; R¹⁰, R^{10a} and each R¹⁶ are independently selected from the group consisting of H, substituted or unsubstituted alkyl, substituted or unsubstituted alkenyl, substituted or unsubstituted alkynyl, substituted or unsubstituted heteroalkyl, substituted or unsubstituted cycloalkyl, substituted or unsubstituted heterocycloalkyl, aryl optionally substituted with one or more, also 1-5, also 1-3, independently selected substituents R²⁷, heteroaryl optionally substituted with one or more, also 1-5, also 1-3, independently selected substituents R²⁷, —CN, -halogen, —OR²⁰, —SR²⁰, —NR²⁰R²¹, —C(O)R²² —C(O)NR²⁰R²¹, —OC(O)NR²⁰R²¹, —C(O)OR²⁰, —NR²³C(O)R²², —NR²³C(O)OR²⁰, —NR²³C(O)NR²⁰R²¹, —NR²³C(S)NR²⁰R²¹, —NR²³S(O)₂R²², —S(O)₂NR²⁰R²¹, —S(O)R²² and —S(O)₂R²²; wherein each occurrence of R²⁰, R²¹ and R²³ are independently selected from the group consisting of H, substituted or unsubstituted C₁-C₆ alkyl, substituted or unsubstituted 3- to 6-membered heteroalkyl, aryl optionally substituted with one or more, also 1-5, also 1-3, independently selected substituents R²⁷, 5- or 6-membered heteroaryl optionally substituted with one or more, also 1-5, also 1-3, independently selected substituents R²⁷, substituted or unsubstituted C₃-C₈ cycloalkyl and substituted or unsubstituted 3- to 8-membered heterocycloalkyl; each occurrence of R²² is independently selected from the group consisting of substituted or unsubstituted C₁-C₆ alkyl, substituted or unsubstituted 3- to 6-membered heteroalkyl, aryl optionally substituted with one or more, also 1-5, also 1-3, independently selected substituents R²⁷, 5- or 6-membered heteroaryl optionally substituted with one or more, also 1-5, also 1-3, independently selected substituents R²⁷, substituted or unsubstituted C₃-C₈ cycloalkyl and substituted or unsubstituted 3- to 8-membered heterocycloalkyl; or any two adjacent R¹⁶, together with the carbon atoms to which they are attached, are optionally joined to form a 5- to 7-membered ring selected from the group consisting of phenyl optionally substituted with one or more, also 1-5, also 1-3, independently selected substituents R²⁷, heteroaryl optionally substituted with one or more, also 1-5, also 1-3, independently selected substituents R²⁷, cycloalkyl optionally substituted with one or more, also 1-5, also 1-3, independently selected substituents R²⁹, and heterocycloalkyl optionally substituted with one or more, also 1-5, also 1-3, independently selected substituents R²⁹; or any two members selected from R¹⁰, R^{10a} and R¹¹, when substituted on adjacent ring atoms, together with the atoms to which they are attached, are optionally joined to form a 5- to 7-membered ring selected from the group consisting of phenyl optionally substituted with one or more, also 1-5, also 1-3, independently selected substituents R²⁷, heteroaryl optionally substituted with one or more, also 1-5, also 1-3, independently selected substituents R²⁷, cycloalkyl optionally substituted with one or more, also 1-5, also 1-3, independently selected substituents R²⁹, and heterocycloalkyl optionally substituted with one or more, also 1-5, also 1-3, independently selected substituents R²⁹; wherein R²⁷ and R²⁹ are as defined for Formula (III).

In one embodiment, the compound of Formula (I) has a structure according to Formula (VI):

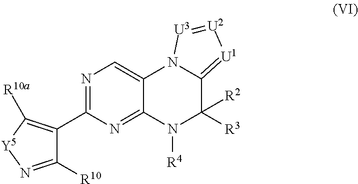

or a salt or solvate thereof, wherein U¹, U², U³, R², R³, and R⁴ are defined as for Formula (I), and Y⁵, R¹⁰ and R^{10a} are defined as for Formula (V) above.

In one embodiment, the compound of Formula (I) has a structure according to Formula (VII):

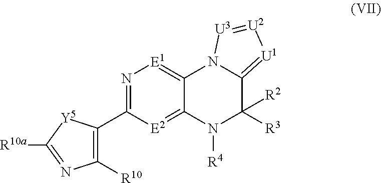

or a salt or solvate thereof, wherein U¹, U², U³, R², R³, and R⁴ are defined as for Formula (I), and Y⁵, R¹⁰ and R^{10a} are defined as for Formula (V) above.

In one example according to any of the above embodiments of Formula (I) to (VII), R² is H. In one embodiment, the compound of Formula (I) has a structure according to Formula (VIII); in one embodiment, the compound of Formula (I) has a structure according to Formula (VIIIa); or in one embodiment, the compound of Formula (I) has a structure according to Formula (VIIIb). In one embodiment, the compound of Formula (I) has a structure selected from the group consisting of Formula (VIIIa) and Formula (VIIIb):

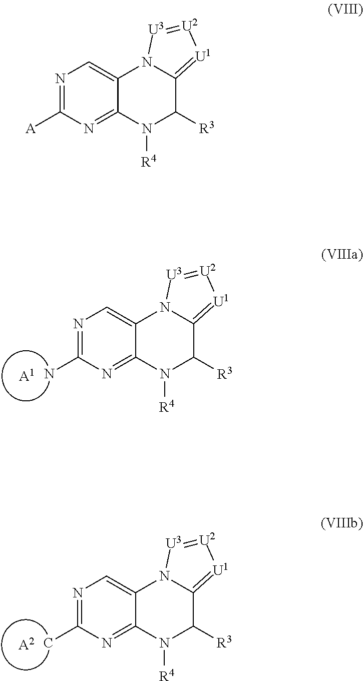

or a salt or solvate thereof, wherein A, U¹, U², U³, R³, and R⁴ are defined as for Formula (I) and A¹ and A² are defined as for Formula (II) and Formula (V), respectively.

In one example, according to any of the above embodiments of Formula (I) to (VII), R⁴ and R³ taken together with the atoms to which they are bound are joined to form a substituted or unsubstituted 5-, or 6-membered heterocylic ring. In one embodiment, the compound of Formula (I) has a structure according to Formula (IX); in one embodiment, the compound of Formula (I) has a structure according to Formula (IXa); or in one embodiment, the compound of Formula (I) has a structure according to Formula (IXb). In one embodiment, the compound of Formula (I) has a structure selected from the group consisting of Formula (IXa) and Formula (IXb):

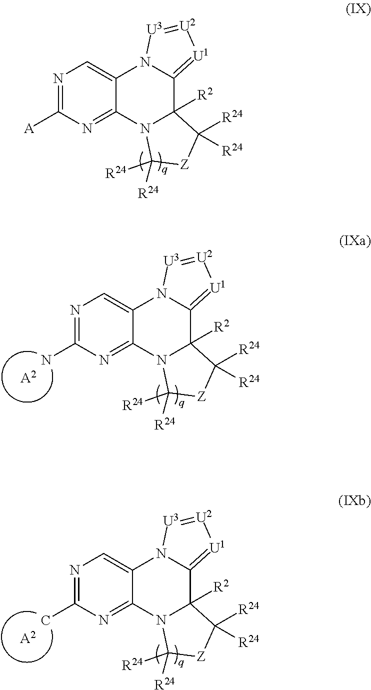

or a salt or solvate thereof, wherein A, U¹, U², U³, and R² are defined as for Formula (I), above; q is 1 or 2, Z is O, N(R⁶⁷), or C(R²⁴)₂, and each R²⁴ is independently H, fluoro, unsubstituted C₁-C₄ alkyl, or C₁-C₄ haloalkyl, R⁶⁷ is H, —C(O)R⁶⁸, —C(O)OR⁶⁸, unsubstituted C₃-C₆ cycloalkyl or unsubstituted C₁-C₄ alkyl, and R⁶⁸ is unsubstituted C₁-C₄ alkyl.

In one example according to any of the above embodiments of Formula (I) to (VII), U³ is CR^{1b}. In one example, the compound of Formula (I) has a structure according to Formula (X); in one example, the compound of Formula (I) has a structure according to Formula (Xa); or in one example, the compound of Formula (I) has a structure according to Formula (Xb). In one embodiment, the compound of Formula (I) has a structure selected from the group consisting of Formula (Xa) and Formula (Xb):

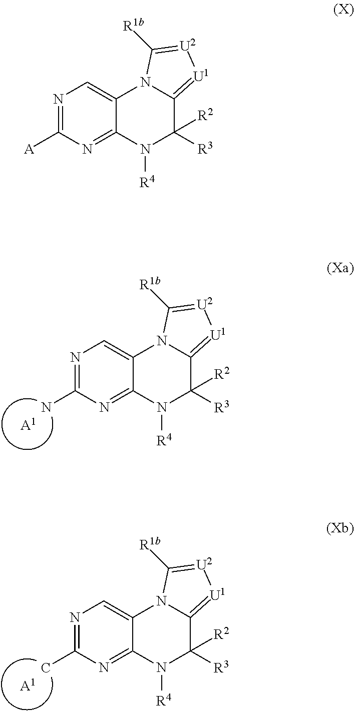

or a salt or solvate thereof, wherein A, U¹, U², R^{1b}, R², R³ and R⁴ are defined as for Formula (I) and A¹ and A² are defined as for Formula (II) and Formula (V), respectively.

In one example according to any of the above embodiments of Formula (I) to (V), U¹ and U² are N, and U³ is CR^{1b}. In one example, the compound of Formula (I) has a structure according to Formula (XI); in one example, the compound of Formula (I) has a structure according to Formula (XIa); or in one example, the compound of Formula (I) has a structure according to Formula (XIb). In one embodiment, the compound of Formula (I) has a structure selected from the group consisting of Formula (XIa) and Formula (XIb):

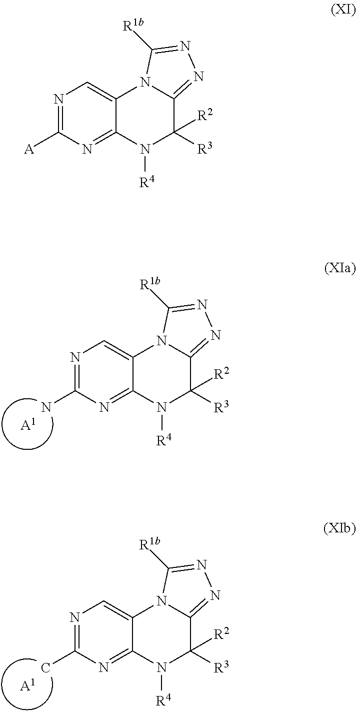

or a salt or solvate thereof, wherein A, R^{1b}, R², R³, and R⁴ are defined as for Formula (I) and A¹ and A² are defined as for Formula (II) and Formula (V), respectively.

In one embodiment, the compound of Formula (I) has a structure according to Formula (XIIa); In one embodiment, the compound of Formula (I) has a structure according to Formula (XIIb); In one embodiment, the compound of Formula (I) has a structure according to Formula (XIIc); In one embodiment, the compound of Formula (I) has a structure according to Formula (XIId); In one embodiment, the compound of Formula (I) has a structure according to Formula (XIIe); or In one embodiment, the compound of Formula (I) has a structure according to Formula (XIIf). In one embodiment, the compound of Formula (I) has a structure selected from the group consisting of Formula (XIIa), Formula (XIIb), Formula (XIIc), Formula (XIId), Formula (XIIe), and Formula (XIIf):

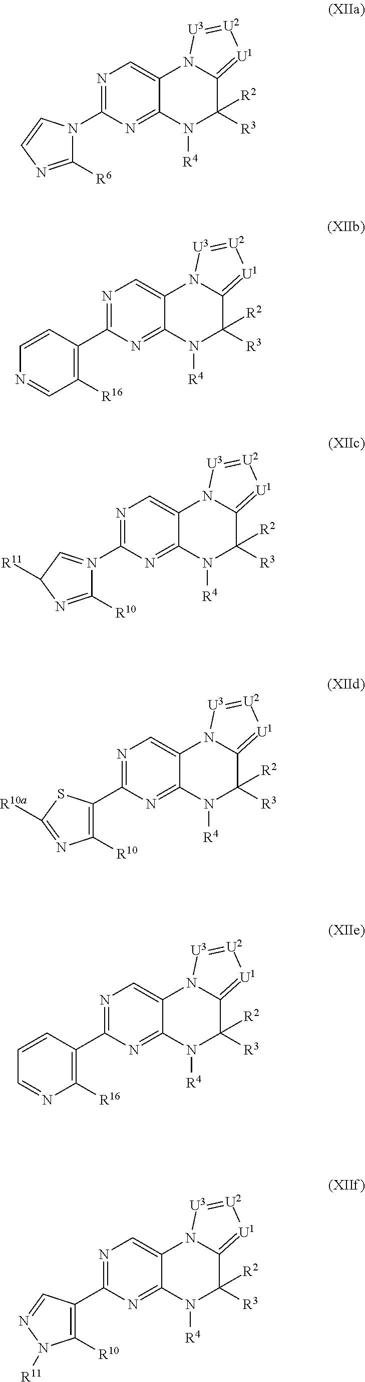

or a salt or solvate thereof, wherein U¹, U², U³, R², R³, and R⁴ are defined as for Formula (I), R⁶ is as defined for Formula (III), and R¹⁰, R^{10a}, R¹¹ and R¹⁶ are as defined for Formula (V), above.

In one embodiment, the compound of Formula (I) has a structure according to Formula (XIIIa); in one embodiment, the compound of Formula (I) has a structure according to Formula (XIIIb); in one embodiment, the compound of Formula (I) has a structure according to Formula (XIIIc); in one embodiment, the compound of Formula (I) has a structure according to Formula (XIIId); in one embodiment, the compound of Formula (I) has a structure according to Formula (XIIIe); or in one embodiment, the compound of Formula (I) has a structure according to Formula (XIIIf). In one embodiment, the compound of Formula (I) has a structure selected from the group consisting of Formula (XIIIa), Formula (XIIIb), Formula (XIIIc), Formula (XIIId), Formula (XIIIe), and Formula (XIIIf):

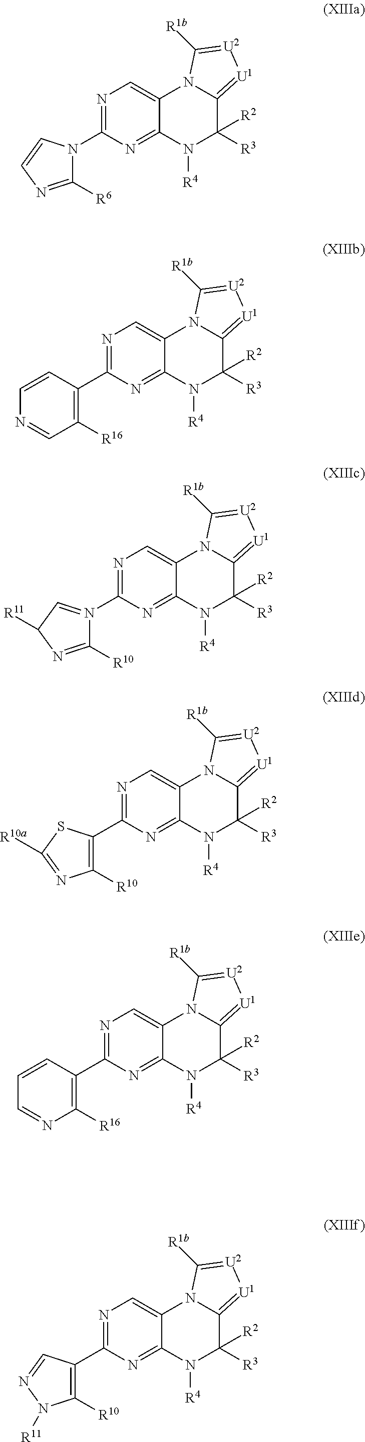

or a salt or solvate thereof, wherein U¹, U², R^{1b}, R², R³, and R⁴ are defined as for Formula (I), R⁶ is as defined for Formula (III), and R¹⁰, R^{10a}, R¹¹ and R¹⁶ are as defined for Formula (V), above.

In one embodiment, the compound of Formula (I) has a structure according to Formula (XIVa); in one embodiment, the compound of Formula (I) has a structure according to Formula (XIVb); in one embodiment, the compound of Formula (I) has a structure according to Formula (XIVc); in one embodiment, the compound of Formula (I) has a structure according to Formula (XIVd); in one embodiment, the compound of Formula (I) has a structure according to Formula (XIVe); or in one embodiment, the compound of Formula (I) has a structure according to Formula (XIVf). In one embodiment, the compound of Formula (I) has a structure selected from the group consisting of Formula (XIVa), Formula (XIVb), Formula (XIVc), Formula (XIVd), Formula (XIVe), and Formula (XIVf):

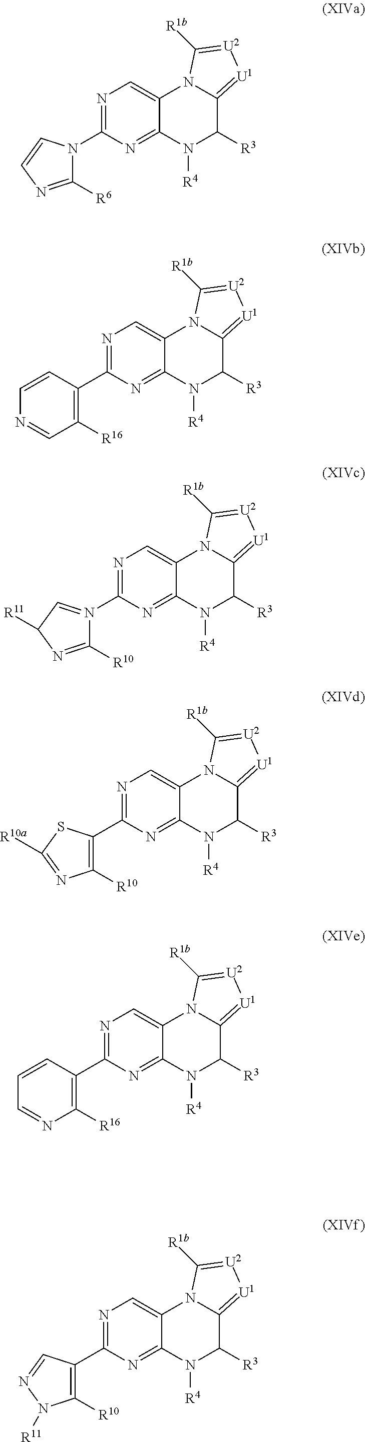

or a salt or solvate thereof, wherein U¹, U², R^{1b}, R³ and R⁴ are defined as for Formula (I), R⁶ is defined as for Formula (III), and R¹⁰, R^{10a}, R¹¹ and R¹⁶ are as defined for Formula (V), above.

In one embodiment, the compound of Formula (I) has a structure according to Formula (XVa); in one embodiment, the compound of Formula (I) has a structure according to Formula (XVb); in one embodiment, the compound of Formula (I) has a structure according to Formula (XVc); in one embodiment, the compound of Formula (I) has a structure according to Formula (XVd); in one embodiment, the compound of Formula (I) has a structure according to Formula (XVe); or in one embodiment, the compound of Formula (I) has a structure according to Formula (XVf). In one embodiment, the compound of Formula (I) has a structure selected from the group consisting of Formula (XVa), Formula (XVb), Formula (XVc), Formula (XVd), Formula (XVe), and Formula (XVf):

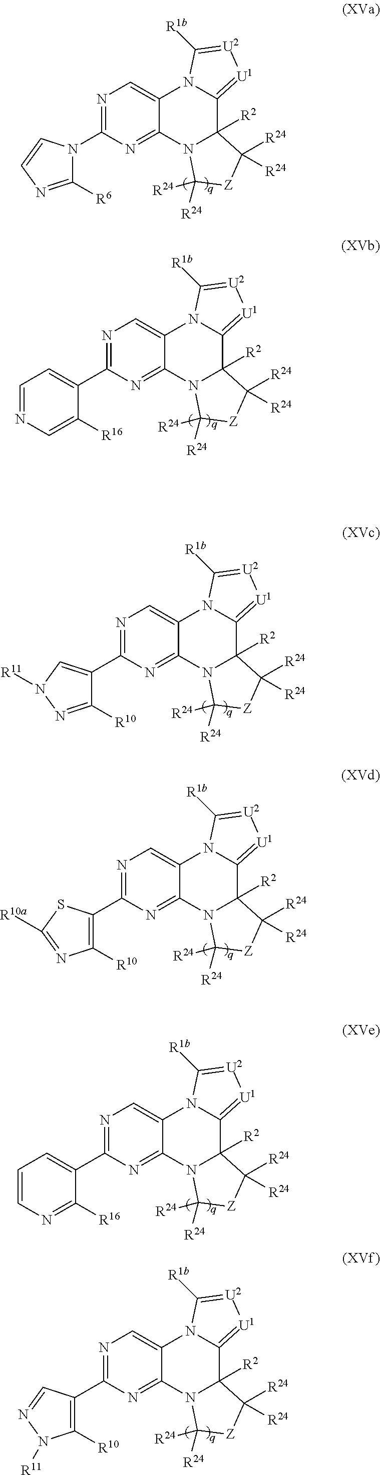

or a salt or solvate thereof, wherein U¹, U², R^{1b}, and R² are defined as for Formula (I), R⁶ is as defined for Formula (III), and R¹⁰, R^{10a}, R¹¹ and R¹⁶ are as defined for Formula (V), and Z, q and R²⁴ are as defined for Formula (IX), above.

In one embodiment, compounds as described herein will have a preferred stereoisomer at the carbon bound to R² and R³ as follows (using Formula (I) for demonstration, the preferred stereoisomer applies to all Formulae as described herein): when R² is H and R³ is selected from the group consisting of substituted or unsubstituted C₁-C₆ alkyl, substituted or unsubstituted C₂-C₆ alkenyl, substituted or unsubstituted C₂-C₆ alkynyl, substituted or unsubstituted 3- to 6-membered heteroalkyl, substituted or unsubstituted C₃-C₆ cycloalkyl and substituted or unsubstituted 3- to 6-membered heterocycloalkyl (preferably when R³ is —CD₃, —CH₃, —CD₂CD₃, —CH₂CH₃, —CH₂-cyclopropyl, or —CH₂CF₃, preferably, —CD₂CD₃, —CH₂CH₃, or —CH₂CF₃) the preferred isomer is represented by the following structure Formula (Ia):

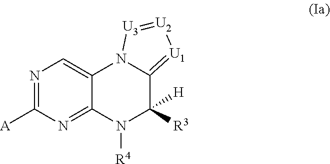

and when R² is selected from the group consisting of substituted or unsubstituted C₁-C₆ alkyl, substituted or unsubstituted C₂-C₆ alkenyl, substituted or unsubstituted C₂-C₆ alkynyl, substituted or unsubstituted 3- to 6-membered heteroalkyl, substituted or unsubstituted C₃-C₆ cycloalkyl and substituted or unsubstituted 3- to 6-membered heterocycloalkyl (preferably when R² is —CD₃, —CH₃, —CD₂CD₃, —CH₂CH₃, —CH₂-cyclopropyl, or —CH₂CF₃, preferably, —CD₂CD₃, —CH₂CH₃, or —CH₂CF₃), and R³ and R⁴, together with the atoms to which they are attached, combine to form a substituted or unsubstituted 3- to 8-membered heterocyclic ring, the preferred isomer is represented by the following structure Formula (Ib), where the dotted line connecting R³ and R⁴ represents a ring as provided in Formula (I) above:

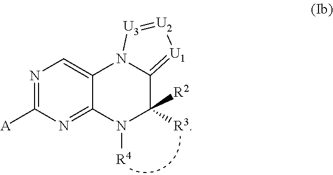

The compounds as represented by Formula I, including all embodiments therein above, also encompass the following embodiments of the various substituents, i.e. A, U₁, U₂, U₃, R², R³ and R⁴, and all sub-embodiment thereof. It is understood that all embodiments of these variables apply to all relevant Formulae (i.e. Formula (I), (II), (III), (IV), (V), (VI), (VII), (VIII), (VIIIa), (VIIIb), (IX), (IXa), (IXb), (X), (Xa), (Xb), (XI), (XIa), (XIb), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), (XIVf), (XVa), (XVb), (XVc), (XVd), (XVe), or (XVf)) and also to any combination of the various embodiments for one variable with any other variable, as applied to all relevant Formulae.

### Ring A

In one example, ring A in Formula (I), (VIII), (IX), (X), or (XI), is a substituted or unsubstituted ring selected from pyrrolidinyl, piperidinyl, morpholinyl, thiomorpholinyl, N-alkyl-piperazinyl, oxazolidinyl, thiazolidinyl, pyridyl, pyrimidinyl, pyridazinyl, pyrazinyl, triazinyl, pyrrolyl, imidazolyl, pyrazolyl, thiazolyl, isothiazolyl, oxazolyl, isoxazolyl, thiadiazolyl, triazolyl and tetrazolyl. In one example, ring A is a substituted or unsubstituted ring selected from pyridyl, pyrimidinyl, pyridazinyl, pyrazinyl, triazinyl, pyrrolyl, imidazolyl, pyrazolyl, thiazolyl, isothiazolyl, oxazolyl, isoxazolyl, thiadiazolyl, triazolyl and tetrazolyl. In one example, ring A is a substituted or unsubstituted ring selected from pyridyl, imidazolyl, pyrazolyl, triazolyl, thiazolyl, isothiazolyl, oxazolyl, and isoxazolyl. In a particular example, ring A is substituted or unsubstituted imidazolyl. In a particular example, ring A is substituted or unsubstituted pyrazolyl. In a particular example, ring A is substituted or unsubstituted thiazolyl. In a particular example, ring A is substituted or unsubstituted pyridyl. In a particular example, ring A is a substituted or unsubstituted ring selected from the group consisting of pyridyl, pyrazolyl and imidazolyl, preferably pyridin-3-yl, pyridin-4-yl, pyrazol-4-yl and imidazol-1-yl.

In one example, ring A¹ in Formula (II), (VIIIa), (IXa), (Xa), or (XIa) is a substituted or unsubstituted ring selected from the group consisting of pyrrolidinyl, piperidinyl, morpholinyl, thiomorpholinyl, N-alkyl-piperazinyl, oxazolidinyl, thiazolidinyl, pyrrolyl, imidazolyl, pyrazolyl, triazolyl and tetrazolyl. In a particular example, ring A¹ is substituted or unsubstituted imidazolyl.

In one example, ring A² in Formula (V), (VIIIb), (IXb), (Xb), or (XIb), is a substituted or unsubstituted ring selected from the group consisting of pyrrolidinyl, piperidinyl, morpholinyl, thiomorpholinyl, N-alkyl-piperazinyl, oxazolidinyl, thiazolidinyl, pyrrolidinyl, pyridyl, pyrimidinyl, pyridazinyl, pyrazinyl, triazinyl, pyrrolyl, imidazolyl, pyrazolyl, thiazolyl, isothiazolyl, oxazolyl, isoxazolyl, thiadiazolyl, triazolyl and tetrazolyl. In one example, ring A² is a substituted or unsubstituted ring selected from the group consisting of pyridyl, pyrimidinyl, pyridazinyl, pyrazinyl, triazinyl, pyrrolyl, imidazolyl, pyrazolyl, thiazolyl, isothiazolyl, oxazolyl, isoxazolyl, thiadiazolyl, triazolyl and tetrazolyl. In one example, ring A² is a substituted or unsubstituted ring selected from the group consisting of pyridyl, imidazolyl, pyrazolyl, thiazolyl, isothiazolyl, oxazolyl, and isoxazolyl. In a particular example, ring A² is a substituted or unsubstituted ring selected from the group consisting of imidazolyl, pyrazolyl, pyrrolyl, triazolyl, tetrazolyl, oxazolyl, thiazolyl and 4-pyridyl. In a particular example, ring A² is substituted or unsubstituted imidazolyl. In a particular example, ring A² is substituted or unsubstituted pyrazolyl. In a particular example, ring A² is substituted or unsubstituted thiazolyl. In a particular example, ring A² is substituted or unsubstituted pyridyl. In a particular example, ring A² is a substituted or unsubstituted ring selected from the group consisting of pyridyl and pyrazolyl, preferably pyridin-3-yl, pyridin-4-yl, and pyrazol-4-yl.

In one example, for ring A in Formula (I), (VIII), (IX), (X), or (XI), ring A¹ in Formula (II), (VIIIa), (IXa), (Xa), or (XIa), or ring A² in Formula (V), (VIIIb), (IXb), (Xb), or (XIb), when the ring is 5- or 6-membered heterocycloalkyl, the ring is optionally substituted with one or more, preferably 1-3, substituents independently selected from the group consisting of C₁-C₆ alkyl optionally substituted with one or more, also 1-5, also 1-3, independently selected substituents R³⁸, 3- to 8-membered heteroalkyl optionally substituted with one or more, also 1-3, independently selected substituents R³⁸, C₃-C₈ cycloalkyl optionally substituted with one or more, also 1-3, independently selected substituents R³⁹, 3- to 8-membered heterocycloalkyl optionally substituted with one or more, also 1-3, independently selected substituents R³⁹, aryl optionally substituted with one or more, also 1-3, independently selected substituents R³⁹, heteroaryl optionally substituted with one or more, also 1-3, independently selected substituents R³⁹, halogen, —CN, ═O, —OR⁴⁰, —SR⁴⁰, ═NR⁴⁰, —NR⁴⁰R⁴¹, —C(O)R⁴², —C(O)OR⁴⁰, —C(O)NR⁴⁰R⁴¹, —NR⁴³C(O)R⁴², —S(O)₂R⁴², —S(O)₂NR⁴⁰R⁴¹, and —NR⁴³S(O)₂R⁴²; R³⁸ at each occurrence is independently selected from the group consisting of —OR⁴⁴, —SR⁴⁴, —NHR⁴⁴, —NR⁴⁴R⁴⁵, —C(O)R⁴⁴, —C(O)OR⁴⁴, —NHC(O)R⁴⁴, —C(O)NHR⁴⁵, —C(O)NR⁴⁴R⁴⁵, —S(O)₂R⁴⁴, —NHS(O)₂R⁴⁴, —S(O)₂NHR⁴⁵, —S(O)₂NR⁴⁴R⁴⁵, -halogen, —C(O)OH, —C(O)NH₂, —CN, —OH, and —NH₂; R³⁹ at each occurrence is independently —R³⁸ or —R⁴⁴; R⁴⁴ and R⁴⁵ are independently C₁-C₄ alkyl optionally substituted with one or more, also 1-5, also 1-3, independently selected substituents selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁴⁶R⁴⁷; or —NR⁴⁴R⁴⁵ forms a 5-, 6-, or 7-membered heterocycloalkyl optionally substituted with one or more, also 1-3, unsubstituted C₁-C₄ alkyl; wherein —NR⁴⁶R⁴⁷ forms a 5-, 6-, or 7-membered heterocycloalkyl optionally substituted with one or more, also 1-3, unsubstituted C₁-C₄ alkyl; and when the ring is aryl or 5- or 6-membered heteroaryl, the ring is optionally substituted with one or more, preferably 1-3, substituents independently selected from the group consisting of C₁-C₆ alkyl optionally substituted with one or more, also 1-5, also 1-3, independently selected substituents R²⁸, C₂-C₆ alkenyl optionally substituted with one or more, also 1-3, independently selected substituents R²⁸, C₂-C₆ alkynyl optionally substituted with one or more, also 1-3, independently selected substituents R²⁸, 3- to 8-membered heteroalkyl optionally substituted with one or more, also 1-3, independently selected substituents R²⁸, C₃-C₈ cycloalkyl optionally substituted with one or more, also 1-3, independently selected substituents R²⁹, 3- to 8-membered heterocycloalkyl optionally substituted with one or more, also 1-3, independently selected substituents R²⁹, aryl optionally substituted with one or more, also 1-3, independently selected substituents R²⁷, heteroaryl optionally substituted with one or more, also 1-3, independently selected substituents R²⁷, —CN, —NO₂, -halogen, —OR¹², —SR¹², —NR¹²R¹³, —C(O)R¹⁴, —C(O)NR¹²R¹³, —OC(O)NR¹²R¹³, —C(O)OR¹², —NR¹⁵C(O)R¹⁴, —NR¹⁵C(O)OR¹², —NR¹⁵C(O)NR¹²R¹³, —NR¹⁵C(S)NR¹²R¹³, —NR¹⁵S(O)₂R¹⁴, —S(O)₂NR¹²R¹³, —S(O)R¹⁴ and —S(O)₂R¹⁴, wherein each occurrence of R¹², R¹³ and R¹⁵ are independently selected from the group consisting of H, C₁-C₆ alkyl optionally substituted with one or more, also 1-5, also 1-3, independently selected substituents R²⁸, 3- to 6-membered heteroalkyl optionally substituted with one or more, also 1-3, independently selected substituents R²⁸, aryl optionally substituted with one or more, also 1-3, independently selected substituents R²⁷, 5- or 6-membered heteroaryl optionally substituted with one or more, also 1-3, independently selected substituents R²⁷, C₃-C₈ cycloalkyl optionally substituted with one or more, also 1-3, independently selected substituents R²⁹, and 3- to 8-membered heterocycloalkyl optionally substituted with one or more, also 1-3, independently selected substituents R²⁹; each occurrence of R¹⁴ is independently selected from the group consisting of C₁-C₆ alkyl optionally substituted with one or more, also 1-5, also 1-3, independently selected substituents R²⁸, 3- to 6-membered heteroalkyl optionally substituted with one or more, also 1-3, independently selected substituents R²⁸, aryl optionally substituted with one or more, also 1-3, independently selected substituents R²⁷, 5- or 6-membered heteroaryl optionally substituted with one or more, also 1-3, independently selected substituents R²⁷, C₃-C₈ cycloalkyl optionally substituted with one or more, also 1-3, independently selected substituents R²⁹, and 3- to 8-membered heterocycloalkyl optionally substituted with one or more, also 1-3, independently selected substituents R²⁹; where R²⁷, R²⁸ and R²⁹ are as defined for Formula (III) above.

In one example, for ring A in Formula (I), (VIII), (IX), (X), or (XI), ring A¹ in Formula (II), (VIIIa), (IXa), (Xa), or (XIa), or ring A² in Formula (V), (VIIIb), (IXb), (Xb), or (XIb), when the ring is 5- or 6-membered heterocycloalkyl, the ring is optionally substituted with one or more, preferably 1-3, substituents independently selected from the group consisting of C₁-C₆ alkyl optionally substituted with one or more, also 1-5, also 1-3, independently selected substituents R³⁸, 3- to 8-membered heteroalkyl optionally substituted with one or more, also 1-3, independently selected substituents R³⁸, C₃-C₆ cycloalkyl optionally substituted with one or more, also 1-3, independently selected substituents R³⁹, 3- to 8-membered heterocycloalkyl optionally substituted with one or more, also 1-3, independently selected substituents R³⁹, aryl optionally substituted with one or more, also 1-3, independently selected substituents R³⁹, heteroaryl optionally substituted with one or more, also 1-3, independently selected substituents R³⁹, halogen, —CN, —OR⁴⁰, —SR⁴⁰, —NR⁴⁰R⁴¹, —C(O)R⁴², —C(O)OR⁴⁰, —C(O)NR⁴⁰R⁴¹, —NR⁴³C(O)R⁴², —S(O)₂R⁴², —S(O)₂NR⁴⁰R⁴¹, and —NR⁴³S(O)₂R⁴²; and when the ring is aryl or 5- or 6-membered heteroaryl, the ring is optionally substituted with one or more, preferably 1-3, substituents independently selected from the group consisting of C₁-C₆ alkyl optionally substituted with one or more, also 1-5, also 1-3, independently selected substituents R³⁸, C₂-C₆ alkenyl optionally substituted with one or more, also 1-3, independently selected substituents R³⁸, C₂-C₆ alkynyl optionally substituted with one or more, also 1-3, independently selected substituents R³⁸, 3- to 8-membered heteroalkyl optionally substituted with one or more, also 1-3, independently selected substituents R³⁸, C₃-C₆ cycloalkyl optionally substituted with one or more, also 1-3, independently selected substituents R³⁹, 3- to 8-membered heterocycloalkyl optionally substituted with one or more, also 1-3, independently selected substituents R³⁹, aryl optionally substituted with one or more, also 1-3, independently selected substituents R³⁹, heteroaryl optionally substituted with one or more, also 1-3, independently selected substituents R³⁹, —CN, —NO₂, halogen, —OR⁴⁰, —SR⁴⁰, —NR⁴⁰R⁴¹, —C(O)R⁴², —C(O)OR⁴⁰, —C(O)NR⁴⁰R⁴¹, —NR⁴³C(O)R⁴², —S(O)₂R⁴², —S(O)₂NR⁴⁰R⁴¹, and —NR⁴³S(O)₂R⁴²; where R⁴⁰, R⁴¹, R⁴², and R⁴³, at each occurrence are independently selected from the group consisting of hydrogen, C₁-C₆ alkyl optionally substituted with one or more, also 1-5, also 1-3, independently selected substituents R³⁸, 3- to 6-membered heteroalkyl optionally substituted with one or more, also 1-3, independently selected substituents R³⁸, C₃-C₆ cycloalkyl optionally substituted with one or more, also 1-3, independently selected substituents R³⁹, 3- to 8-membered heterocycloalkyl optionally substituted with one or more, also 1-3, independently selected substituents R³⁹, aryl optionally substituted with one or more, also 1-3, independently selected substituents R³⁹, and heteroaryl optionally substituted with one or more, also 1-3, independently selected substituents R³⁹, provided that R⁴² is other than hydrogen; R³⁸ at each occurrence is independently selected from the group consisting of —OR⁴⁴, —SR⁴⁴ —NHR⁴⁴, —NR⁴⁴R⁴⁵, —C(O)R⁴⁴, —C(O)OR⁴⁴, —NHC(O)R⁴⁴, —C(O)NHR⁴⁵, —C(O)NR⁴⁴R⁴⁵, —S(O)₂R⁴⁴, —NHS(O)₂R⁴⁴, —S(O)₂NHR⁴⁵, —S(O)₂NR⁴⁴R⁴⁵, -halogen, —C(O)OH, —C(O)NH₂, —CN, —OH, and —NH₂; R³⁹ at each occurrence is independently —R³⁸ or —R⁴⁴; R⁴⁴ and R⁴⁵ are independently C₁-C₄ alkyl optionally substituted with one or more, also 1-5, also 1-3, substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁴⁶R⁴⁷; or —NR⁴⁴R⁴⁵ forms a 5-, 6-, or 7-membered heterocycloalkyl optionally substituted with one or more, also 1-3, unsubstituted C₁-C₄ alkyl; wherein —NR⁴⁶R⁴⁷ forms a 5-, 6-, or 7-membered heterocycloalkyl optionally substituted with one or more, also 1-3, unsubstituted C₁-C₄ alkyl.

In one example, for ring A in Formula (I), (VIII), (IX), (X), or (XI), ring A¹ in Formula (II), (VIIIa), (IXa), (Xa), or (XIa), or ring A² in Formula (V), (VIIIb), (IXb), (Xb), or (XIb), when the ring is 5- or 6-membered heterocycloalkyl, the ring is optionally substituted with one or more, preferably 1-3, substituents independently selected from the group consisting of C₁-C₆ alkyl optionally substituted with one or more, also 1-5, also 1-3, independently selected substituents R³⁸, phenyl optionally substituted with one or more, also 1-3, independently selected substituents R³⁹, 5- or 6-membered heteroaryl optionally substituted with one or more, also 1-3, independently selected substituents R³⁹, fluoro, —OR⁴⁰, —SR⁴⁰, —NR⁴⁰R⁴¹, —C(O)R⁴², —C(O)NR⁴⁰R⁴¹, —S(O)₂R⁴², and —S(O)₂NR⁴⁰R⁴¹; and when the ring is aryl or 5- or 6-membered heteroaryl, the ring is optionally substituted with one or more, preferably 1-3, substituents independently selected from the group consisting of C₁-C₆ alkyl optionally substituted with one or more, also 1-5, also 1-3, independently selected substituents R³⁸, C₃-C₆ cycloalkyl optionally substituted with one or more, also 1-3, independently selected substituents R³⁹, 3- to 8-membered heterocycloalkyl optionally substituted with one or more, also 1-3, independently selected substituents R³⁹, phenyl optionally substituted with one or more, also 1-3, independently selected substituents R³⁹, 5- or 6-membered heteroaryl optionally substituted with one or more, also 1-3, independently selected substituents R³⁹, —CN, —NO₂, halogen, —OR⁴⁰, —SR⁴⁰, —NR⁴⁰R⁴¹, —C(O)R⁴², —NR⁴³C(O)R⁴², —C(O)_{NR}⁴⁰R⁴¹, —S(O)₂R⁴², —NR⁴³S(O)₂R⁴², and —S(O)₂NR⁴⁰R⁴¹. Wherein for the examples in this paragraph, R³⁸ at each occurrence is independently —OR⁴⁴, —NHR⁴⁴, —NR⁴⁴R⁴⁵, -halogen, —CN, —OH, or —NH₂; R³⁹ at each occurrence is independently —R³⁸ or —R⁴⁴; R⁴⁰, R⁴¹, R⁴², and R⁴³, at each occurrence are independently hydrogen or C₁-C₆ alkyl optionally substituted with one or more, also 1-5, also 1-3, independently selected substituents R³⁸; R⁴⁴ and R⁴⁵ are independently C₁-C₄ alkyl optionally substituted with one or more substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁴⁶R⁴⁷; or —NR⁴⁴R⁴⁵ forms a 5-, 6-, or 7-membered heterocycloalkyl optionally substituted with one or more, also 1-3, unsubstituted C₁-C₄ alkyl; wherein —NR⁴⁶R⁴⁷ forms a 5-, 6-, or 7-membered heterocycloalkyl optionally substituted with one or more, also 1-3, unsubstituted C₁-C₄ alkyl.

In one example, for ring A in Formula (I), (VIII), (IX), (X), or (XI), or ring A² in Formula (V), (VIIIb), (IXb), (Xb), or (XIb), the ring A is phenyl or 5- or 6-membered heteroaryl, the ring A² is 5- or 6-membered heteroaryl, and the ring is substituted with one substituent selected from the group consisting of —NHC(O)phenyl, —S(O)₂CH₃, 5- or 6-membered unsubstituted cycloalkyl, 5- or 6-membered unsubstituted heterocycloalkyl, aryl optionally substituted with one or more, also 1-3, independently selected substituents R²⁷ and heteroaryl optionally substituted with one or more, also 1-3, independently selected substituents R²⁷, and the ring is further optionally substituted with 1-2 substituents independently selected from the group consisting of C₁-C₆ alkyl optionally substituted with one or more, also 1-5, also 1-3, independently selected substituents R³⁸, halogen, —CN, —OR⁴⁰, —SR⁴⁰, —NR⁴⁰R⁴¹, —C(O)R⁴², —C(O)OR⁴⁰, —C(O)NR⁴⁰R⁴¹, —NR⁴³C(O)R⁴², —S(O)₂R⁴², —S(O)₂NR⁴⁰R⁴¹, and —NR⁴³S(O)₂R⁴². In one example, the ring A is phenyl or 5- or 6-membered heteroaryl, the ring A² is 5- or 6-membered heteroaryl, and the ring is substituted with one substituent selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with one or more, also 1-3, independently selected substituents R²⁷ and 5- or 6-membered heteroaryl optionally substituted with one or more, also 1-3, independently selected substituents R²⁷, and the ring is further optionally substituted with 1-2 substituents independently selected from the group consisting of C₁-C₆ alkyl optionally substituted with one or more, also 1-5, also 1-3, independently selected substituents R³⁸, halogen, —CN, —OR⁴⁰, —SR⁴⁰, —NR⁴⁰R⁴¹, —C(O)R⁴², —C(O)OR⁴⁰, —C(O)NR⁴⁰R⁴¹, —NR⁴³C(O)R⁴², —S(O)₂R⁴², —S(O)NR⁴⁰R⁴¹ and —NR⁴³S(O)₂R⁴². In one example, the ring A is phenyl or 5- or 6-membered heteroaryl, the ring A² is 5- or 6-membered heteroaryl, and the ring is substituted with one substituent selected from the group consisting of —NHC(O)phenyl, —S(O)₂CH₃, 5- or 6-membered unsubstituted cycloalkyl, 5- or 6-membered unsubstituted heterocycloalkyl, aryl optionally substituted with one or more, also 1-3, independently selected substituents R³⁹ and heteroaryl optionally substituted with one or more, also 1-3, independently selected substituents R³⁹, and the ring is further optionally substituted with 1-2 substituents independently selected from the group consisting of C₁-C₆ alkyl optionally substituted with one or more, also 1-5, also 1-3, independently selected substituents R³⁸, halogen, —CN, —OR⁴⁰, —SR⁴⁰, —NR⁴⁰R⁴¹, —C(O)R⁴², —C(O)OR⁴⁰, —C(O)NR⁴⁰R⁴¹, —NR⁴³C(O)R⁴², —S(O)₂R⁴², —S(O)₂NR⁴⁰R⁴¹, and —NR⁴³S(O)₂R⁴². In one example, the ring A is phenyl or 5- or 6-membered heteroaryl, the ring A² is 5- or 6-membered heteroaryl, and the ring is substituted with one substituent selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with one or more, also 1-3, independently selected substituents R³⁹ or 5- or 6-membered heteroaryl optionally substituted with one or more, also 1-3, independently selected substituents R³⁹, and the ring is further optionally substituted with 1-2 substituents independently selected from the group consisting of C₁-C₆ alkyl optionally substituted with one or more, also 1-5, also 1-3, independently selected substituents R³⁸, halogen, —CN, —OR⁴⁰, —SR⁴⁰, —NR⁴⁰R⁴¹, —C(O)R⁴², —C(O)OR⁴⁰, —C(O)NR⁴⁰R⁴¹, —NR⁴³C(O)R⁴², —S(O)₂R⁴², —S(O)₂NR⁴⁰R⁴¹, and —NR⁴³S(O)₂R⁴². Wherein for the examples in this paragraph, R²⁷ is as defined for Formula (III); R³⁸ at each occurrence is independently —OR⁴⁴, —NHR⁴⁴, —NR⁴⁴R⁴⁵, -halogen, —CN, —OH, or —NH₂; R³⁹ at each occurrence is independently —R³⁸ or —R⁴⁴; R⁴⁰, R⁴¹, R⁴², and R⁴³, at each occurrence are independently hydrogen or C₁-C₆ alkyl optionally substituted with one or more, also 1-5, also 1-3, independently selected substituents R³⁸; R⁴⁴ and R⁴⁵ are independently C₁-C₄ alkyl optionally substituted with one or more substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁴⁶R⁴⁷; or —NR⁴⁴R⁴⁵ forms a 5-, 6-, or 7-membered heterocycloalkyl optionally substituted with one or more, also 1-3, unsubstituted C₁-C₄ alkyl; wherein —NR⁴⁶R⁴⁷ forms a 5-, 6-, or 7-membered heterocycloalkyl optionally substituted with one or more, also 1-3, unsubstituted C₁-C₄ alkyl.

In one example, for ring A in Formula (I), (VIII), (IX), (X), or (XI), or ring A² in Formula (V), (VIIIb), (IXb), (Xb), or (XIb), the ring A is phenyl or 5- or 6-membered heteroaryl, the ring A² is 5- or 6-membered heteroaryl, and the ring is substituted with one substituent selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with one or more, also 1-3, substituents independently selected from the group consisting of halogen, —CN, unsubstituted C₁-C₄ alkyl, C₁-C₄ haloalkyl, —OR⁷⁰, and —S(O)₂R⁷⁰, and heteroaryl optionally substituted with one or more, also 1-3, substituents independently selected from the group consisting of halogen, unsubstituted C₁-C₄ alkyl, and C₁-C₄ haloalkyl, and the ring is further optionally substituted with 1-2 substituents independently selected from the group consisting of unsubstituted C₁-C₄ alkyl, C₁-C₄ haloalkyl, halogen, —CN, —OR⁷¹, —NR⁷¹R⁷², —C(O)R⁷³, —C(O)NR⁷¹R⁷², —NHC(O)R⁷³, —S(O)₂R⁷³, —S(O)₂NR⁷¹R⁷², and —NHS(O)₂R⁷³; wherein R⁷⁰, R⁷¹, R⁷², and R⁷³ are independently unsubstituted C₁-C₄ alkyl or C₁-C₄ haloalkyl. In one example, the ring A is phenyl or 5- or 6-membered heteroaryl, the ring A² is 5- or 6-membered heteroaryl, and the ring is substituted with one substituent selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with one or more, also 1-3, substituents independently selected from the group consisting of halogen, —CN, unsubstituted C₁-C₄ alkyl, C₁-C₄ haloalkyl, —OR⁷⁰, and —S(O)₂R⁷⁰, and heteroaryl optionally substituted with one or more, also 1-3, substituents independently selected from the group consisting of halogen, unsubstituted C₁-C₄ alkyl, and C₁-C₄ haloalkyl; wherein R⁷⁰ is unsubstituted C₁-C₄ alkyl or C₁-C₄ haloalkyl. In one example, the ring A or A² is 5- or 6-membered heteroaryl substituted with one substituent selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with 1-2 substituents independently selected from the group consisting of —F, —Cl, —Br, —CN, —CF₃, and —OCF₃, and heteroaryl optionally substituted with 1-2 fluoro, where preferably ring A or A² is pyridine-4-yl, imidazole, thiazole, isothiazole, pyrazole or triazole substituted with one substituent selected from the group consisting of phenyl optionally substituted with 1-2 substituents independently selected from the group consisting of —F, —Cl, —Br, —CN, —CF₃, and —OCF₃, pyridine optionally substituted with 1-2 fluoro, pyrimidine optionally substituted with 1-2 fluoro, thiazole, oxazole, and pyrazole.

In one example, in Formula (II), (VIII a), (IXa), (Xa), or (XIa), ring A¹ is 5-membered heteroaryl substituted with one substituent selected from the group consisting of —NHC(O)phenyl, —S(O)₂CH₃, 5- or 6-membered unsubstituted cycloalkyl, 5- or 6-membered unsubstituted heterocycloalkyl, aryl optionally substituted with one or more substituents R²⁷ and heteroaryl optionally substituted with one or more substituents R²⁷, and the ring is further optionally substituted with 1-2 substituents independently selected from the group consisting of C₁-C₆ alkyl optionally substituted with one or more, also 1-5, from the group consisting of C₁-C₆ alkyl optionally substituted with one or more, also 1-5, also 1-3, independently selected substituents R³⁸, halogen, —CN, —OR⁴⁰, —SR⁴⁰, —NR⁴⁰R⁴¹, —C(O)R⁴², —C(O)OR⁴⁰, —C(O)NR⁴⁰R⁴¹, —NR⁴³C(O)R⁴², —S(O)₂R⁴², —S(O)₂NR⁴⁰R⁴¹, and —NR⁴³S(O)₂R⁴². In one example, ring A¹ is 5-membered heteroaryl substituted with one substituent selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with one or more, also 1-3, independently selected substituents R²⁷ and 5- or 6-membered heteroaryl optionally substituted with one or more, also 1-3, independently selected substituents R²⁷, and the ring is further optionally substituted with 1-2 substituents independently selected from the group consisting of C₁-C₆ alkyl optionally substituted with one or more, also 1-5, also 1-3, independently selected substituents R³⁸, halogen, —CN, —OR⁴⁰, —SR⁴⁰, —NR⁴⁰R⁴¹, —C(O)R⁴², —C(O)OR⁴⁰, —C(O)NR⁴⁰R⁴¹, —NR⁴³C(O)R⁴², —S(O)₂R⁴², —S(O)₂NR⁴⁰R⁴¹, and —NR⁴³S(O)₂R⁴². In one example, ring A¹ is 5-membered heteroaryl substituted with one substituent selected from the group consisting of —NHC(O)phenyl, —S(O)₂CH₃, 5- or 6-membered unsubstituted cycloalkyl, 5- or 6-membered unsubstituted heterocycloalkyl,aryl optionally substituted with one or more, also 1-3, independently selected substituents R³⁹ and heteroaryl optionally substituted with one or more, also 1-3, independently selected substituents R³⁹, and the ring is further optionally substituted with 1-2 substituents independently selected from the group consisting of C₁-C₆ alkyl optionally substituted with one or more, also 1-5, also 1-3, independently selected substituents R³⁸, halogen, —CN, —OR⁴⁰, —SR⁴⁰, —NR⁴⁰R⁴¹, —C(O)R⁴², —C(O)OR⁴⁰, —C(O)NR⁴⁰R⁴¹, —NR⁴³C(O)R⁴², —S(O)₂R⁴², —S(O)₂NR⁴⁰R⁴¹, and —NR⁴³S(O)₂R⁴². In one example, ring A¹ is 5-membered heteroaryl substituted with one substituent selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with one or more, also 1-3, independently selected substituents R³⁹ or 5- or 6-membered heteroaryl optionally substituted with one or more, also 1-3, independently selected substituents R³⁹, and the ring is further optionally substituted with 1-2 substituents independently selected from the group consisting of C₁-C₆ alkyl optionally substituted with one or more, also 1-5, also 1-3, independently selected substituents R³⁸, halogen, —CN, —OR⁴⁰, —SR⁴⁰, —NR⁴⁰R⁴¹, —C(O)R⁴², —C(O)OR⁴⁰, —C(O)NR⁴⁰R⁴¹; —NR⁴³C(O)R⁴², —S(O)₂NR⁴⁰R⁴¹, and —NR⁴³S(O)₂R⁴². Wherein for the examples in this paragraph, R²⁷ is as defined for Formula (III); R³⁸ at each occurrence is independently —OR⁴⁴; —NHR⁴⁴, —NR⁴⁴R⁴⁵; -halogen, —CN, —OH, or —NH₂; R³⁹ at each occurrence is independently —R³⁸ or —R⁴⁴; R⁴⁰; R⁴¹, R⁴², and R⁴³, at each occurrence are independently hydrogen or C₁-C₆ alkyl optionally substituted with one or more, also 1-5, also 1-3, independently selected substituents R³⁸; R⁴⁴ and R⁴⁵ are independently C₁-C₄ alkyl optionally substituted with one or more substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁴⁶R⁴⁷; or —NR⁴⁴R⁴⁵ forms a 5-, 6-, or 7-membered heterocycloalkyl optionally substituted with one or more, also 1-3, unsubstituted C₁-C₄ alkyl; wherein —NR⁴⁶R⁴⁷ forms a 5-, 6-, or 7-membered heterocycloalkyl optionally substituted with one or more, also 1-3, unsubstituted C₁-C₄ alkyl.

In one example, in Formula (II), (VIIIa), (IXa), (Xa), or (XIa), ring A¹ is 5-membered heteroaryl substituted with one substituent selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with one or more, also 1-3, substituents independently selected from the group consisting of halogen, —CN, unsubstituted C₁-C₄ alkyl, C₁-C₄ haloalkyl, —OR⁷⁰, and —S(O)₂R⁷⁰, and heteroaryl optionally substituted with one or more, also 1-3, substituents independently selected from the group consisting of halogen, unsubstituted C₁-C₄ alkyl, and C₁-C₄ haloalkyl, and the ring is further optionally substituted with 1-2 substituents independently selected from the group consisting of unsubstituted C₁-C₄ alkyl, C₁-C₄ haloalkyl, halogen, —CN, —OR⁷¹, —NR⁷¹R⁷²₅—C(O)R⁷³, —C(O)NR⁷¹R⁷², —NHC(O)R⁷³, —S(O)₂R⁷³, —S(O)₂NR⁷¹R⁷², and —NHS(O)₂R⁷³; wherein R⁷⁰, R⁷¹, R⁷², and R⁷³ are independently unsubstituted C₁-C₄ alkyl or C₁-C₄ haloalkyl. In one example, the ring is 5-membered heteroaryl substituted with one substituent selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with one or more, also 1-3, substituents independently selected from the group consisting of halogen, —CN, unsubstituted C₁-C₄ alkyl, C₁-C₄ haloalkyl, —OR⁷⁰, and —S(O)₂R⁷⁰, and heteroaryl optionally substituted with one or more, also 1-3, substituents independently selected from the group consisting of halogen, unsubstituted C₁-C₄ alkyl, and C₁-C₄ haloalkyl; wherein R⁷⁰ is unsubstituted C₁-C₄ alkyl or C₁-C₄ haloalkyl. In one example, the ring is 5-membered heteroaryl substituted with one substituent selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with 1-2 substituents independently selected from the group consisting of —F, —Cl, —Br, —CN, —CF₃, and —OCF₃, and heteroaryl optionally substituted with 1-2 fluoro, where preferably A¹ is imidazole, pyrazole, or triazole, more preferably imidazole substituted with one substituent selected from the group consisting of phenyl optionally substituted with 1-2 substituents independently selected from the group consisting of —F, —Cl, —Br, —CN, —CF₃, and —OCF₃, pyridine optionally substituted with 1-2 fluoro, pyrimidine optionally substituted with 1-2 fluoro, thiazole, oxazole, and pyrazole.

In one example, in Formula (III), (IV), (VI), (VII), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), (XIVf), (XVa), (XVb), (XVc), (XVd), (XVe), or (XVf), each occurrence of R⁶, R⁷, R⁸, R⁹, R¹⁰, R^{10a}, or R¹⁶ are independently selected from the group consisting of H, substituted or unsubstituted C₁-C₁₀ alkyl, substituted or unsubstituted C₂-C₁₀ alkenyl, substituted or unsubstituted C₂-C₁₀ alkynyl, substituted or unsubstituted 3- to 10-membered heteroalkyl, substituted or unsubstituted C₃-C₈ cycloalkyl, substituted or unsubstituted 3- to 8-membered heterocycloalkyl, phenyl optionally substituted with one or more, also 1-3, independently selected substituents R²⁷, 5- or 6-membered heteroaryl optionally substituted with one or more, also 1-3, independently selected substituents R²⁷, —CN, -halogen, —OR¹², —SR¹², —NR¹²R¹³, —C(O)R¹⁴, —C(O)NR¹²R¹³, —OC(O)NR¹²R¹³, —C(O)OR¹², —NR¹⁵C(O)R¹⁴, —NR¹⁵C(O)OR¹², —NR¹⁵C(O)NR¹²R¹³, —NR¹⁵C(S)NR¹²R¹³, —NR¹⁵S(O)₂R¹⁴, —S(O)₂NR¹²R¹³, —S(O)R¹⁴ and —S(O)₂R¹⁴; or any two of R⁶, R⁷, R⁸ or R⁹ are optionally joined to form a 3- to 7-membered ring selected from the group consisting of phenyl optionally substituted with one or more, also 1-3, independently selected substituents R²⁷, 5- or 6-membered heteroaryl optionally substituted with one or more, also 1-3, independently selected substituents R²⁷, C₃-C₈ cycloalkyl optionally substituted with one or more, also 1-3, independently selected substituents R²⁹, and 3- to 8-membered heterocycloalkyl optionally substituted with one or more, also 1-3, independently selected substituents R²⁹; or any two of R¹⁰, R^{10a} or R¹¹, when on adjacent ring atoms, or any two R¹⁶, when on adjacent ring atoms, together with the atoms to which they are attached, are optionally joined to form a 5- to 7-membered ring selected from the group consisting of phenyl optionally substituted with one or more, also 1-3, independently selected substituents R²⁷, heteroaryl optionally substituted with one or more, also 1-3, independently selected substituents R²⁷, cycloalkyl optionally substituted with one or more, also 1-3, independently selected substituents R²⁹, and heterocycloalkyl optionally substituted with one or more, also 1-3, independently selected substituents R²⁹; each occurrence of R¹¹ is independently selected from the group consisting of H, —C(O)R²², substituted or unsubstituted C₁-C₆ alkyl, substituted or unsubstituted 3- to 6-membered heteroalkyl, aryl optionally substituted with one or more, also 1-3, independently selected substituents R²⁷, 5- or 6-membered heteroaryl optionally substituted with one or more, also 1-3, independently selected substituents R²⁷, C₃-C₈ cycloalkyl optionally substituted with one or more, also 1-3, independently selected substituents R²⁹, and 3- to 8-membered heterocycloalkyl optionally substituted with one or more, also 1-3, independently selected substituents R²⁹; and R²² is independently selected from the group consisting of substituted or unsubstituted C₁-C₆ alkyl, substituted or unsubstituted 3- to 6-membered heteroalkyl, aryl optionally substituted with one or more, also 1-3, independently selected substituents R²⁷, 5- or 6-membered heteroaryl optionally substituted with one or more, also 1-3, independently selected substituents R²⁷, C₃-C₈ cycloalkyl optionally substituted with one or more, also 1-3, independently selected substituents R²⁹, and 3- to 8-membered heterocycloalkyl optionally substituted with one or more, also 1-3, independently selected substituents R²⁹; where R²⁷, R²⁸ and R²⁹ are as defined for Formula (III) above.

In one example, in Formula (III), (IV), (VI), (VII), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), (XIVf), (XVa), (XVb), (XVc), (XVd), (XVe), or (XVf), each occurrence of R⁶, R⁷, R⁸, R⁹, R¹⁰, R^{10a}, or R¹⁶ are independently selected from the group consisting of H, C₁-C₆ alkyl optionally substituted with one or more, also 1-5, also 1-3, independently selected substituents R²⁸, C₂-C₆ alkenyl optionally substituted with one or more, also 1-3, independently selected substituents R²⁸, C₂-C₆ alkynyl optionally substituted with one or more, also 1-3, independently selected substituents R²⁸, 3- to 8-membered heteroalkyl optionally substituted with one or more, also 1-3, independently selected substituents R²⁸, C₃-C₈ cycloalkyl optionally substituted with one or more, also 1-3, independently selected substituents R²⁹, 3- to 8-membered heterocycloalkyl optionally substituted with one or more, also 1-3, independently selected substituents R²⁹, aryl optionally substituted with one or more, also 1-3, independently selected substituents R²⁷, heteroaryl optionally substituted with one or more, also 1-3, independently selected substituents R²⁷, —CN, -halogen, —OR¹², —SR¹², —NR¹²R¹³, —C(O)R¹⁴, —C(O)NR¹²R¹³, —OC(O)NR¹²R¹³, —C(O)OR¹², —NR¹⁵C(O)R¹⁴, —NR¹⁵C(O)OR¹², —NR¹⁵C(O)NR¹²R¹³, —NR¹⁵S(O)₂R¹⁴, —S(O)₂NR¹²R¹³, —S(O)R¹⁴ and —S(O)₂R¹⁴, wherein each occurrence of R¹², R¹³ and R¹⁵ are independently selected from the group consisting of H, C₁-C₆ alkyl optionally substituted with one or more, also 1-5, also 1-3, independently selected substituents R²⁸, 3- to 6-membered heteroalkyl optionally substituted with one or more, also 1-3, independently selected substituents R²⁸, aryl optionally substituted with one or more, also 1-3, independently selected substituents R²⁷, 5- or 6-membered heteroaryl optionally substituted with one or more, also 1-3, independently selected substituents R²⁷, C₃-C₈ cycloalkyl optionally substituted with one or more, also 1-3, independently selected substituents R²⁹, and 3- to 8-membered heterocycloalkyl optionally substituted with one or more, also 1-3, independently selected substituents R²⁹; each occurrence of R¹⁴ is independently selected from the group consisting of C₁-C₆ alkyl optionally substituted with one or more, also 1-5, also 1-3, independently selected substituents R²⁸, 3- to 6-membered heteroalkyl optionally substituted with one or more, also 1-3, independently selected substituents R²⁸, aryl optionally substituted with one or more, also 1-3, independently selected substituents R²⁷, 5- or 6-membered heteroaryl optionally substituted with one or more, also 1-3, independently selected substituents R²⁷, C₃-C₈ cycloalkyl optionally substituted with one or more, also 1-3, independently selected substituents R²⁹, and 3- to 8-membered heterocycloalkyl optionally substituted with one or more, also 1-3, independently selected substituents R²⁹; each occurrence of R¹¹ is independently selected from the group consisting of H, —C(O)R²², substituted or unsubstituted C₁-C₆ alkyl, substituted or unsubstituted 3- to 6-membered heteroalkyl, aryl optionally substituted with one or more, also 1-3, independently selected substituents R²⁷, 5- or 6-membered heteroaryl optionally substituted with one or more, also 1-3, independently selected substituents R²⁷, C₃-C₈ cycloalkyl optionally substituted with one or more, also 1-3, independently selected substituents R²⁹, and 3- to 8-membered heterocycloalkyl optionally substituted with one or more, also 1-3, independently selected substituents R²⁹; and R²² is independently selected from the group consisting of substituted or unsubstituted C₁-C₆ alkyl, substituted or unsubstituted 3- to 6-membered heteroalkyl, aryl optionally substituted with one or more, also 1-3, independently selected substituents R²⁷, 5- or 6-membered heteroaryl optionally substituted with one or more, also 1-3, independently selected substituents R²⁷, C₃-C₈ cycloalkyl optionally substituted with one or more, also 1-3, independently selected substituents R²⁹, and 3- to 8-membered heterocycloalkyl optionally substituted with one or more, also 1-3, independently selected substituents R²⁹; where R²⁷, R²⁸ and R²⁹ are as defined for Formula (III) above.

In one example, in Formula (III), (IV), (VI), (VII), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), (XIVf), (XVa), (XVb), (XVc), (XVd), (XVe), or (XVf), each occurrence of R⁶, R⁷, R⁸, R⁹, R¹⁰_{, R}^{10a}, or R¹⁶ are independently selected from the group consisting of H, C₁-C₆ alkyl optionally substituted with one or more, also 1-5, also 1-3, independently selected substituents R³⁸, C₂-C₆ alkenyl optionally substituted with one or more, also 1-3, independently selected substituents R³⁸, C₂-C₆ alkynyl optionally substituted with one or more, also 1-3, independently selected substituents R³⁸, 3- to 8-membered heteroalkyl optionally substituted with one or more, also 1-3, independently selected substituents R³⁸, C₃-C₆ cycloalkyl optionally substituted with one or more, also 1-3, independently selected substituents R³⁹, 3- to 8-membered heterocycloalkyl optionally substituted with one or more, also 1-3, independently selected substituents R³⁹, aryl optionally substituted with one or more, also 1-3, independently selected substituents R³⁹, heteroaryl optionally substituted with one or more, also 1-3, independently selected substituents R³⁹, —CN, —NO₂, halogen, —OR⁴⁰, —SR⁴⁰, —NR⁴⁰R⁴¹, —C(O)R⁴², —C(O)OR⁴⁰, —C(O)NR⁴⁰R⁴¹, —NR⁴³C(O)R⁴², —S(O)₂R⁴², —S(O)₂NR⁴⁰R⁴¹, and —NR⁴³S(O)₂R⁴²; where R⁴⁰, R⁴¹, R⁴² and R⁴³, at each occurrence are independently selected from the group consisting of hydrogen, C₁-C₆ alkyl optionally substituted with one or more, also 1-5, also 1-3, independently selected substituents R³⁸, 3- to 6-membered heteroalkyl optionally substituted with one or more, also 1-3, independently selected substituents R³⁸, C₃-C₈ cycloalkyl optionally substituted with one or more, also 1-3, independently selected substituents R³⁹, 3- to 8-membered heterocycloalkyl optionally substituted with one or more, also 1-3, independently selected substituents R³⁹, aryl optionally substituted with one or more, also 1-3, independently selected substituents substituents R³⁹, and heteroaryl optionally substituted with one or more, also 1-3, independently selected substituents substituents R³⁹, provided that R⁴² is other than hydrogen; R³⁸ at each occurrence is independently selected from the group consisting of —OR⁴⁴, —SR⁴⁴, —NHR⁴⁴, —NR⁴⁴R⁴⁵, —C(O)R⁴⁴, —C(O)OR⁴⁴, —NHC(O)R⁴⁴, —C(O)NHR⁴⁵, —C(O)NR⁴⁴R⁴⁵, —S(O)₂R⁴⁴, —NHS(O)₂R⁴⁴, —S(O)₂NHR⁴⁵, —S(O)₂NR⁴⁴R⁴⁵, -halogen, —C(O)OH, —C(O)NH₂, —CN, —OH, and —NH₂; R³⁹ at each occurrence is independently —R³⁸ or —R⁴⁴; R⁴⁴ and R⁴⁵ are independently C₁-C₄ alkyl optionally substituted with one or more, also 1-5, also 1-3, independently selected substituents selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁴⁶R⁴⁷; or —NR⁴⁴R⁴⁵ forms a 5-, 6-, or 7-membered heterocycloalkyl optionally substituted with one or more, also 1-3, unsubstituted C₁-C₄ alkyl; wherein —NR⁴⁶R⁴⁷ forms a 5-, 6-, or 7-membered heterocycloalkyl optionally substituted with one or more, also 1-3, unsubstituted C₁-C₄ alkyl; each occurrence of R¹¹ is independently selected from the group consisting of H, —C(O)R²², C₁-C₆ alkyl optionally substituted with one or more R³⁸, 3- to 6-membered heteroalkyl optionally substituted with one or more R³⁸, aryl optionally substituted with one or more R³⁹, 5- or 6-membered heteroaryl optionally substituted with one or more R³⁹, C₃-C₈ cycloalkyl optionally substituted with one or more R³⁹, and 3- to 8-membered heterocycloalkyl optionally substituted with one or more R³⁹; and R²² is independently selected from the group consisting of C₁-C₆ alkyl optionally substituted with one or more R³⁸, 3- to 6-membered heteroalkyl optionally substituted with one or more R³⁸, aryl optionally substituted with one or more substituents R³⁹, 5- or 6-membered heteroaryl optionally substituted with one or more substituents R³⁹, C₃-C₈ cycloalkyl optionally substituted with one or more R³⁹, and 3- to 8-membered heterocycloalkyl optionally substituted with one or more R³⁹.

In one example, in Formula (III), (IV), (VI), (VII), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), (XIVf), (XVa), (XVb), (XVc), (XVd), (XVe), or (XVf), each occurrence of R⁶, R⁷, R⁸, R⁹, R¹⁰, R^{10a}, or R¹⁶ are in dependently selected from the group consisting of H, C₁-C₆ alkyl optionally substituted with one or more, also 1-5, also 1-3, independently selected substituents R³⁸, C₃-C₆ cycloalkyl optionally substituted with one or more, also 1-3, independently selected substituents R³⁹, 3- to 8-membered heterocycloalkyl optionally substituted with one or more, also 1-3, independently selected substituents R³⁹, phenyl optionally substituted with one or more, also 1-3, independently selected substituents R³⁹, 5- or 6-membered heteroaryl optionally substituted with one or more, also 1-3, independently selected substituents R³⁹, —CN, —NO₂, halogen, —OR⁴⁰, —SR⁴⁰, —NR⁴⁰R⁴¹—C(O)R⁴², —NR⁴³C(O)R⁴², —C(O)NR⁴⁰R⁴¹, —S(O)₂R⁴², —NR⁴³S(O)₂R⁴², and —S(O)₂NR⁴⁰R⁴¹ each occurrence of R¹¹ is independently selected from the group consisting of H, —C(O)R²², C₁-C₆ alkyl optionally substituted with one or more R³⁸, 3- to 6-membered heteroalkyl optionally substituted with one or more R³⁸, aryl optionally substituted with one or more R³⁹, 5- or 6-membered heteroaryl optionally substituted with one or more R³⁹, C₃-C₈ cycloalkyl optionally substituted with one or more R³⁹, and 3- to 8-membered heterocycloalkyl optionally substituted with one or more R³⁹; and R²² is independently selected from the group consisting of C₁-C₆ alkyl optionally substituted with one or more R³⁸, 3- to 6-membered heteroalkyl optionally substituted with one or more R³⁸, aryl optionally substituted with one or more substituents R³⁹, 5- or 6-membered heteroaryl optionally substituted with one or more substituents R³⁹, C₃-C₈ cycloalkyl optionally substituted with one or more R³⁹, and 3- to 8-membered heterocycloalkyl optionally substituted with one or more R³⁹. Wherein for the examples in this paragraph, R³⁸ at each occurrence is independently —OR⁴⁴, —NHR⁴⁴, —NR⁴⁴R⁴⁵, -halogen, —CN, —OH, or —NH₂; R³⁹ at each occurrence is independently —R³⁸ or —R⁴⁴; R⁴⁰, R⁴¹, R⁴², and R⁴³, at each occurrence are independently hydrogen or C₁-C₆ alkyl optionally substituted with one or more, also 1-5, also 1-3, independently selected substituents R³⁸; R⁴⁴ and R⁴⁵ are independently C₁-C₄ alkyl optionally substituted with one or more substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁴⁶R⁴⁷; or —NR⁴⁴R⁴⁵ forms a 5-, 6-, or 7-membered heterocycloalkyl optionally substituted with one or more, also 1-3, unsubstituted C₁-C₄ alkyl; wherein —NR⁴⁶R⁴⁷ forms a 5-, 6-, or 7-membered heterocycloalkyl optionally substituted with one or more, also 1-3, unsubstituted C₁-C₄ alkyl.

In one example, in Formula (III), (IV), (VI), (VII), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), (XIVf), (XVa), (XVb), (XVc), (XVd), (XVe), or (XVf), each occurrence of R⁶, R¹⁰, and R¹⁶ are independently selected from the group consisting of —NHC(O)phenyl, —S(O)₂CH₃, 5- or 6-membered unsubstituted cycloalkyl, 5- or 6-membered unsubstituted heterocycloalkyl, aryl optionally substituted with one or more substituents R²⁷ or and heteroaryl optionally substituted with one or more substituents R²⁷; each occurrence of R⁷, R⁸, R⁹, and R^{10a} are independently selected from the group consisting of H, C₁-C₆ alkyl optionally substituted with one or more, also 1-5, also 1-3, independently selected substituents R³⁸, halogen, —CN, —OR⁴⁰, —SR⁴⁰, —NR⁴⁰R⁴¹, —C(O)R⁴², —C(O)OR⁴⁰, —C(O)NR⁴⁰R⁴¹, —NR⁴³C(O)R⁴², —S(O)₂R⁴², —S(O)₂NR⁴⁰R⁴¹, and —NR⁴³S(O)₂R⁴²; and each occurrence of R¹¹ is independently selected from the group consisting of H, —C(O)R²², C₁-C₆ alkyl optionally substituted with one or more R³⁸, 3- to 6-membered heteroalkyl optionally substituted with one or more R³⁸, aryl optionally substituted with one or more R³⁹, 5- or 6-membered heteroaryl optionally substituted with one or more R³⁹, C₃-C₈ cycloalkyl optionally substituted with one or more R³⁹, and 3- to 8-membered heterocycloalkyl optionally substituted with one or more R³⁹; and R²² is independently selected from the group consisting of C₁-C₆ alkyl optionally substituted with one or more R³⁸, 3- to 6-membered heteroalkyl optionally substituted with one or more R³⁸, aryl optionally substituted with one or more substituents R³⁹, 5- or 6-membered heteroaryl optionally substituted with one or more substituents R³⁹, C₃-C₈ cycloalkyl optionally substituted with one or more R³⁹, and 3- to 8-membered heterocycloalkyl optionally substituted with one or more R³⁹. Wherein for the examples in this paragraph, R²⁷ is as defined for Formula (III); R³⁸ at each occurrence is independently —OR⁴⁴, —NHR⁴⁴, —NR⁴⁴R⁴⁵, -halogen, —CN, —OH, or —NH₂; R³⁹ at each occurrence is independently —R³⁸ or and R⁴³, at each occurrence are independently hydrogen or C₁-C₆ alkyl optionally substituted with one or more, also 1-5, also 1-3, independently selected substituents R³⁸; R⁴⁴ and R⁴⁵ are independently C₁-C₄ alkyl optionally substituted with one or more substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁴⁶R⁴⁷; or —NR⁴⁴R⁴⁵ forms a 5-, 6-, or 7-membered heterocycloalkyl optionally substituted with one or more, also 1-3, unsubstituted C₁-C₄ alkyl; wherein —NR⁴⁶R⁴⁷ forms a 5-, 6-, or 7-membered heterocycloalkyl optionally substituted with one or more, also 1-3, unsubstituted C₁-C₄ alkyl.

In one example, in Formula (III), (IV), (VI), (VII), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), (XIVf), (XVa), (XVb), (XVc), (XVd), (XVe), or (XVf), each occurrence of R⁶, R¹⁰, and R¹⁶ are independently selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with one or more, also 1-3, substituents independently selected from the group consisting of halogen, —CN, unsubstituted C₁-C₄ alkyl, C₁-C₄ haloalkyl, —OR⁷⁰, and —S(O)₂R⁷⁰, and heteroaryl optionally substituted with one or more, also 1-3, substituents independently selected from the group consisting of halogen, unsubstituted C₁-C₄ alkyl, and C₁-C₄ haloalkyl; each occurrence of R⁷, R⁸, R⁹, and R^{10a} are independently selected from the group consisting of H, C₁-C₄ alkyl, C₁-C₄ haloalkyl, halogen, —CN, —OR⁷¹, —NR⁷¹R⁷², —C(O)R⁷³, —C(O)NR⁷¹R⁷², —NHC(O)R⁷³, —S(O)₂R⁷³, —S(O)₂NR⁷¹R⁷², and —NHS(O)₂R⁷³; and each occurrence of R¹¹ is independently selected from the group consisting of H, —C(O)R⁷³, unsubstituted C₁-C₄ alkyl, and C₁-C₄ haloalkyl; wherein R⁷⁰, R⁷¹, R⁷², and R⁷³ are independently unsubstituted C₁-C₄ alkyl or C₁-C₄ haloalkyl. In one example each occurrence of R⁶, R¹⁰, R¹⁶ are independently selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with one or more, also 1-3, substituents independently selected from the group consisting of halogen, —CN, unsubstituted C₁-C₄ alkyl, C₁-C₄ haloalkyl, —OR⁷⁰, and —S(O)₂R⁷⁰, and heteroaryl optionally substituted with one or more, also 1-3, substituents independently selected from the group consisting of halogen, unsubstituted C₁-C₄ alkyl, and C₁-C₄ haloalkyl wherein R⁷⁰ is unsubstituted C₁-C₄ alkyl or C₁-C₄ haloalkyl. In one example, each occurrence of R⁶, R¹⁰ and R¹⁶ are independently selected from the group consisting of —NHC(O)phenyl, —S(O)₂CH₃, 5- or 6-membered unsubstituted cycloalkyl, 5- or 6-membered unsubstituted heterocycloalkyl, phenyl optionally substituted with 1-2 substituents independently selected from the group consisting of —F, —Cl, —Br, —CN, —CF₃, and —OCF₃, and heteroaryl optionally substituted with 1-2 fluoro, where preferably each occurrence of R⁶, R¹⁰, and R¹⁶ are independently selected from the group consisting of phenyl optionally substituted with 1-2 substituents independently selected from the group consisting of —F, —Cl, —Br, —CN, —CF₃, and —OCF₃, pyridine optionally substituted with 1-2 fluoro, pyrimidine optionally substituted with 1-2 fluoro, thiazole, oxazole, and pyrazole.

In one example according to any of the above embodiments of Formula (I), (V), (VIII), (VIIIb), (IX), (IXb), (X), (Xb), (XI), or (XIb), ring A or A² is preferably other than 3-pyridinyl or 3,5-pyrimidinyl. In one example, ring A or A² is preferably other than substituted 3-pyridinyl or substituted 3,5-pyrimidinyl.

### Substituent U¹, U² and U³

In one example, regarding embodiments of Formula (I), (II), (III), (IV), (V), (VI), (VII), (VIII), (VIII a), (VIIIb), (IX), (IXa), (IXb), (XIIa), (XIIb), (XIIc), (XIId), (XIIe), or (XIIf), U¹ is N or CR¹, U² is N or CR^{1a} and U³ is CR^{1b}. In one example, U¹ is N, U² is N and U³ is CR^{1b}. In one example, U¹ is CR¹, U² is N and U³ is CR^{1b}. In one example, U¹ is N, U² is CR^{1a} and U³ is CR^{1b}. In one example, U¹ is N or CH, U² is N or CH and U³ is CH. In one example, U¹ is N, U² is N and U³ is CH. In one example, U¹ is CH, U² is N and U³ is CH. In one example, U¹ is N, U² is CH and U³ is CH.

In one example, regarding embodiments of Formula (I), (II), (III), (IV), (V), (VI), (VII), (VIII), (VIII a), (VIIIb), (IX), (IXa), (IXb), (XIIa), (XIIb), (XIIc), (XIId), (XIIe), or (XIIf), U¹ is N or CR¹, U² is CR^{1a} and U³ is N or CR^{1b}. In one example, U¹ is N, U² is CR^{1a} and U³ is N. In one example, U¹ is CR¹, U² is CR^{1a} and U³ is N. In one example, U¹ is N or CH, U² is CH and U³ is N or CH. In one example, U¹ is N, U² is CH and U³ is CH. In one example, U¹ is CH, U² is CH and U³ is N.

In one example, regarding embodiments of Formula (I), (II), (III), (IV), (V), (VI), (VII), (VIII), (VIII a), (VIIIb), (IX), (IXa), (IXb), (XIIa), (XIIb), (XIIc), (XIId), (XIIe), or (XIIf), U¹ is CR¹, U² is N or CR^{1a} and U³ is N or CR^{1b}. In one example, U¹ is CR¹, U² is N and U³ is N. In one example, U¹ is CH, U² is N or CH and U³ is N or CH. In one example, U¹ is CH, U² is N and U³ is N.

In one example, regarding embodiments of Formula (X), (Xa), (Xb), (XIIIa), (XIIIb), (XIIIc), (XIIId), (XIIId), (XIIIe), (XIIIf), (XIVa), (XIVb), (XIVc), (XIVd), (XIVe), (XIVf), (XVa), (XVb), (XVc), (XVd), (XVe), or (XVf), U¹ is CR¹ and U² is N. In one example, U¹ is N and U² is CR^{1a}. In one example, U¹ is N and U² is N. In one example, U¹ is CH and U² is N. In one example, U¹ is N and U² is CH.

In one example, regarding embodiments of Formula (I), (II), (III), (IV), (V), (VI), (VII), (VIII), (VIIIa), (VIIIb), (IX), (IXa), (IXb), (X), (Xa), (Xb), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), (XIVf), (XVa), (XVb), (XVc), (XVd), (XVe), or (XVf), R¹, R^{1a} and R^{1b}, if present, are independently selected from H, fluoro, unsubstituted C₁-C₂ alkyl, and C₁-C₂ haloalkyl.

### Substituents R² and R³

In one example, regarding embodiments of Formula (I), (II), (III), (IV), (V), (VI), (VII), (X), (Xa), (Xb), (XI), (XIa), (XIb), (XIIa), (XIIb), (XIIc), (XIId), (XIIe), (XIIf), (XIIIa), (XIIIb), (XIIIc), (XIIId), (XIIIe), or (XIIIf), R² is selected from the group consisting of H, substituted or unsubstituted C₁-C₄ alkyl, substituted or unsubstituted C₂-C₄ alkenyl, substituted or unsubstituted C₂-C₄ alkynyl, substituted or unsubstituted 3- to 6-membered heteroalkyl, substituted or unsubstituted C₃-C₆ cycloalkyl and substituted or unsubstituted 3- to 6-membered heterocycloalkyl; R³ is selected from the group consisting of substituted or unsubstituted C₁-C₄ alkyl, substituted or unsubstituted C₂-C₄ alkenyl, substituted or unsubstituted C₂-C₄ alkynyl, substituted or unsubstituted 3- to 6-membered heteroalkyl, substituted or unsubstituted C₃-C₆ cycloalkyl and substituted or unsubstituted 3- to 6-membered heterocycloalkyl; or R² and R³, together with the carbon atom to which they are attached, are joined to form a substituted or unsubstituted C₃-C₆ cycloalkyl or a substituted or unsubstituted 3- to 6-membered heterocycloalkyl group; or R⁴ and R³ are joined to form a substituted or unsubstituted 3- to 8-membered heterocyclic ring, and R² is selected from H, substituted or unsubstituted C₁-C₄ alkyl, substituted or unsubstituted C₂-C₄ alkenyl, substituted or unsubstituted C₂-C₄ alkynyl, substituted or unsubstituted 3- to 6-membered heteroalkyl, substituted or unsubstituted C₃-C₆ cycloalkyl and substituted or unsubstituted 3- to 6-membered heterocycloalkyl.

In one example, regarding embodiments of Formula (I), (II), (III), (IV), (V), (VI), (VII), (X), (Xa), (Xb), (XI), (XIa), (XIb), (XIIa), (XIIb), (XIIc), (XIId), (XIIe), (XIIf), (XIIIa), (XIIIb), (XIIIc), (XIIId), (XIIIe), or (XIIIf), R² is selected from the group consisting of H, C₁-C₄ alkyl optionally substituted with one or more, also 1-5, also 1-3, independently selected substituents R⁵³, C₂-C₄ alkenyl optionally substituted with one or more, also 1-3, independently selected substituents R⁵³, C₂-C₄ alkynyl optionally substituted with one or more, also 1-3, independently selected substituents R⁵³, 3- to 6-membered heteroalkyl optionally substituted with one or more, also 1-3, independently selected substituents R⁵³, C₃-C₆ cycloalkyl optionally substituted with one or more, also 1-3, independently selected substituents R⁵⁴, and 3- to 6-membered heterocycloalkyl optionally substituted with one or more, also 1-3, independently selected substituents R⁵⁴; R³ is selected from the group consisting of C₁-C₄ alkyl optionally substituted with one or more, also 1-5, also 1-3, independently selected substituents R⁵³, C₂-C₄ alkenyl optionally substituted with one or more, also 1-3, independently selected substituents R⁵³, C₂-C₄ alkynyl optionally substituted with one or more, also 1-3, independently selected substituents R⁵³, 3- to 6-membered heteroalkyl optionally substituted with one or more, also 1-3, independently selected substituents R⁵³, C₃-C₆ cycloalkyl optionally substituted with one or more, also 1-3, independently selected substituents R⁵⁴, and 3- to 6-membered heterocycloalkyl optionally substituted with one or more, also 1-3, independently selected substituents R⁵⁴; or R² and R³, together with the carbon atom to which they are attached, are joined to form a C₃-C₆ cycloalkyl group optionally substituted with one or more, also 1-3, independently selected substituents R⁵⁴, or a 3- to 6-membered heterocycloalkyl group optionally substituted with one or more, also 1-3, independently selected substituents R⁵⁴; or R⁴ and R³ are joined to form a 3- to 8-membered heterocyclic ring optionally substituted with one or more, also 1-3, independently selected substituents R⁵⁴, and R² is selected from H, C₁-C₄ alkyl optionally substituted with one or more, also 1-5, also 1-3, independently selected substituents R⁵³, C₂-C₄ alkenyl optionally substituted with one or more, also 1-3, independently selected substituents R⁵³, C₂-C₄ alkynyl optionally substituted with one or more, also 1-3, independently selected substituents R⁵³, 3- to 6-membered heteroalkyl optionally substituted with one or more, also 1-3, independently selected substituents R⁵³, C₃-C₆ cycloalkyl optionally substituted with one or more, also 1-3, independently selected substituents R⁵⁴, and 3- to 6-membered heterocycloalkyl optionally substituted with one or more, also 1-3, independently selected substituents R⁵⁴; wherein R⁵³ at each occurrence is independently —OR⁵⁵, —NHR⁵⁵, —NR⁵⁵R⁵⁶, -halogen, —OH, or —NH₂; R″ at each occurrence is independently —R⁵³ or —R⁵⁵; R″ and R⁵⁶ are independently unsubstituted C₃-C₆ cycloalkyl or C₁-C₄ alkyl optionally substituted with one or more, also 1-5, also 1-3, substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁵⁷R⁵⁸; or —NR⁵⁵R⁵⁶ forms a 5-, 6-, or 7-membered heterocycloalkyl optionally substituted with one or more, also 1-3, unsubstituted C₁-C₄ alkyl; wherein —NR⁵⁷R⁵⁸ forms a 5-, 6-, or 7-membered heterocycloalkyl optionally substituted with one or more, also 1-3, unsubstituted C₁-C₄ alkyl.

In one example, regarding embodiments of Formula (I), (II), (III), (IV), (V), (VI), (VII), (X), (Xa), (Xb), (XI), (XIa), (XIb), (XIIa), (XIIb), (XIIc), (XIId), (XIIe), (XIIf), (XIIIa), (XIIIb), (XIIIc), (XIIId), (XIIIe), or (XIIIf), R² is H, unsubstituted C₁-C₄ alkyl or C₁-C₄ haloalkyl, and R³ is unsubstituted C₁-C₄ alkyl or C₁-C₄ haloalkyl; or R² and R³ are joined to form an unsubstituted C₃-C₅ cycloalkyl ring; or R⁴ and R³ together with the atoms to which they are attached are joined to form a 5-, 6-, or 7-membered heterocycloalkyl ring optionally substituted with one or more, also 1-3, substituents independently selected from the group consisting of fluoro, unsubstituted C₃-C₆ cycloalkyl, unsubstituted C₁-C₄ alkyl, and C₁-C₄ haloalkyl, and R² is H, unsubstituted C₁-C₄ alkyl or C₁-C₄ haloalkyl. In one example, R² is H and R³ is ethyl; or R² and R³ are joined to form a cyclopropyl or cyclobutyl ring; or R⁴ and R³ together with the atoms to which they are attached are joined to form a 5-, 6-, or 7-membered heterocycloalkyl ring optionally substituted with one or more, also 1-3, substituents independently selected from the group consisting of fluoro, unsubstituted C₃-C₆ cycloalkyl, unsubstituted C₁-C₄ alkyl, and C₁-C₄ haloalkyl, and R² is H or ethyl. In one example, R⁴ and R³ together with the atoms to which they are attached are joined to form a 5-, 6-, or 7-membered heterocycloalkyl ring optionally substituted with one or more, also 1-3, substituents independently selected from the group consisting of fluoro, unsubstituted C₃-C₆ cycloalkyl, unsubstituted C₁-C₄ alkyl, and C₁-C₄ haloalkyl, and R² is H or ethyl. In one example, R⁴ and R³ together with the atoms to which they are attached form a morpholine, pyrrolidine, piperidine, or piperazine ring, wherein the morpholine, pyrrolidine, piperidine or piperazine ring is optionally substituted with one or more, also 1-3, substituents independently selected from the group consisting of fluoro, unsubstituted C₃-C₆ cycloalkyl, unsubstituted C₁-C₄ alkyl, and C₁-C₄ haloalkyl, and R² is H or ethyl.

In one example, regarding embodiments of Formula (I), (II), (III), (IV), (V), (VI), (VII), (X), (Xa), (Xb), (XI), (XIa), (XIb), (XIIa), (XIIb), (XIIc), (XIId), (XIIe), (XIIf), (XIIIa), (XIIIb), (XIIIc), (XIIId), (XIIIe), or (XIIIf), R² is H and R³ is unsubstituted C₁-C₄ alkyl or C₁-C₄ haloalkyl. In one example, R² is H and R³ is unsubstituted C₁-C₂ alkyl or C₁-C₂ haloalkyl. In one example, R² is H and R³ is ethyl, monofluoroethyl, difluoroethyl or trifluoroethyl. In one example, R² is H and R³ is ethyl. In one example, R² is H and R³ is CH₂CH₃ or CD₂CD₃.

In one example, regarding embodiments of Formula (VIII), (VIIIa), (VIIIb), (XIVa), (XIVb), (XIVc), (XIVd), (XIVe), or (XIVf), R³ is selected from the group consisting of substituted or unsubstituted C₁-C₄ alkyl, substituted or unsubstituted C₂-C₄ alkenyl, substituted or unsubstituted C₂-C₄ alkynyl, substituted or unsubstituted 3- to 6-membered heteroalkyl, substituted or unsubstituted C₃-C₆ cycloalkyl and substituted or unsubstituted 3- to 6-membered heterocycloalkyl; or R⁴ and R³ are joined to form a substituted or unsubstituted 3- to 8-membered heterocyclic ring.

In one example, regarding embodiments of Formula (VIII), (VIIIa), (VIIIb), (XIVa), (XIVb), (XIVc), (XIVd), (XIVe), or (XIVf), R³ is selected from the group consisting of C₁-C₄ alkyl optionally substituted with one or more, also 1-5, also 1-3, independently selected substituents R⁵³, C₂-C₄ alkenyl optionally substituted with one or more, also 1-3, independently selected substituents R⁵³, C₂-C₄ alkynyl optionally substituted with one or more, also 1-3, independently selected substituents R⁵³, 3- to 6-membered heteroalkyl optionally substituted with one or more, also 1-3, independently selected substituents R⁵³, C₃-C₆ cycloalkyl optionally substituted with one or more, also 1-3, independently selected substituents R⁵⁴, and 3- to 6-membered heterocycloalkyl optionally substituted with one or more, also 1-3, independently selected substituents R⁵⁴; or R⁴ and R³ are joined to form a 3- to 8-membered heterocyclic ring optionally substituted with one or more, also 1-3, independently selected substituents R⁵⁴; wherein R⁵³ at each occurrence is independently —OR⁵⁵, —NHR⁵⁵, —NR⁵⁵R⁵⁶, -halogen, —OH, or —NH₂; R⁵⁴ at each occurrence is independently —R⁵³ or —R⁵⁵; R⁵⁵ and R⁵⁶ are independently unsubstituted C₃-C₆ cycloalkyl or C₁-C₄ alkyl optionally substituted with one or more, also 1-5, also 1-3, substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁵⁷R⁵⁸; or —NR⁵⁵R⁵⁶ forms a 5-, 6-, or 7-membered heterocycloalkyl optionally substituted with one or more, also 1-3, unsubstituted C₁-C₄ alkyl; wherein —NR⁵⁷R⁵⁸ forms a 5-, 6-, or 7-membered heterocycloalkyl optionally substituted with one or more, also 1-3, unsubstituted C₁-C₄ alkyl.

In one example, regarding embodiments of Formula (VIII), (VIII a), (VIIIb), (XIVa), (XIVb), (XIVc), (XIVd), (XIVe), or (XIVf), R³ is unsubstituted C₁-C₄ alkyl or C₁-C₄ haloalkyl; or R⁴ and R³ together with the atoms to which they are attached are joined to form a 5-, 6-, or 7-membered heterocycloalkyl ring optionally substituted with one or more, also 1-3, substituents independently selected from the group consisting of fluoro, unsubstituted C₃-C₆ cycloalkyl, unsubstituted C₁-C₄ alkyl, and C₁-C₄ haloalkyl. In one example, R⁴ and R³ together with the atoms to which they are attached are joined to form a 5-, 6-, or 7-membered heterocycloalkyl ring optionally substituted with one or more, also 1-3, substituents independently selected from the group consisting of fluoro, unsubstituted C₃-C₆ cycloalkyl, unsubstituted C₁-C₄ alkyl, and C₁-C₄ haloalkyl. In one example, R⁴ and R³ together with the atoms to which they are attached form a morpholine, pyrrolidine, piperidine, or piperazine ring, wherein the morpholine, pyrrolidine, piperidine, or piperazine ring is optionally substituted with one or more, also 1-3, substituents independently selected from the group consisting of fluoro, unsubstituted C₃-C₆ cycloalkyl, unsubstituted C₁-C₄ alkyl, and C₁-C₄ haloalkyl.

In one example, regarding embodiments of Formula (VIII), (VIII a), (VIIIb), (XIVa), (XIVb), (XIVc), (XIVd), (XIVe), or (XIVf), R³ is unsubstituted C₁-C₄ alkyl or C₁-C₄ haloalkyl. In one example, R³ is unsubstituted C₁-C₂ alkyl or C₁-C₂ haloalkyl. In one example, R³ is ethyl, monofluoroethyl, difluoroethyl or trifluoroethyl. In one example, R³ is ethyl. In one example, R³ is CH₂CH₃ or CD₂CD₃.

In one example, regarding embodiments of Formula (IX), (IXa), (IXb), (XVa), (XVb), (XVc), (XVd), (XVe), or (XVf), R² is H or unsubstituted C₁-C₄ alkyl or C₁-C₄ haloalkyl. In one example, R² is H or unsubstituted C₁-C₂ alkyl or C₁-C₂ haloalkyl. In one example, R² is H or ethyl, monofluoroethyl, difluoroethyl or trifluoroethyl. In one example, R² is H or ethyl. In one example, R² is ethyl, monofluoroethyl, difluoroethyl or trifluoroethyl. In one example, R² ethyl. In one example, R² is CH₂CH₃ or CD₂CD₃.

### Substituent R⁴

In one example, regarding embodiments of Formula (I), (II), (III), (IV), (V), (VI), (VII), (VIII), (VIIIa), (VIIIb), (X), (Xa), (Xb), (XI), (XIa), (XIb), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), or (XIVf), R⁴ is selected from the group consisting of —NR⁶⁵R⁶⁶, C₁-C₁₀ alkyl optionally substituted with one or more, also 1-5, also 1-3, independently selected substituents R⁵⁹, C₂-C₁₀ alkenyl optionally substituted with one or more, also 1-3, independently selected substituents R⁵⁹, C₂-C₁₀ alkynyl optionally substituted with one or more, also 1-3, independently selected substituents R⁵⁹, 3- to 10-membered heteroalkyl optionally substituted with one or more, also 1-3, independently selected substituents R⁵⁹, C₃-C₈ cycloalkyl optionally substituted with one or more, also 1-3, independently selected substituents R⁶⁰, 3- to 8-membered heterocycloalkyl optionally substituted with one or more, also 1-3, independently selected substituents R⁶⁰, phenyl optionally substituted with one or more, also 1-3, independently selected substituents R⁶⁰, and 5 or 6 membered heteroaryl optionally substituted with one or more, also 1-3, independently selected substituents R⁶⁰; or R⁴ and R³, together with the atoms to which they are attached, are joined to form a 3- to 8-membered heterocyclic ring optionally substituted with one or more, also 1-3, independently selected substituents R⁶⁰; wherein R⁵⁹ at each occurrence is independently —OR⁶¹; —NHR⁶¹; —NR⁶¹R⁶²; -halogen, —CN, —OH, or —NH₂; R⁶⁰ at each occurrence is independently —R⁵⁹ or —R⁶¹; R⁶¹ and R⁶² are independently unsubstituted C₃-C₆ cycloalkyl or C₁-C₄ alkyl optionally substituted with one or more, also 1-5, also 1-3, substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁶³R⁶⁴; or —NR⁶¹R⁶² forms a 5-, 6-, or 7-membered heterocycloalkyl optionally substituted with one or more, also 1-3, unsubstituted C₁-C₄ alkyl; wherein —NR⁶³R⁶⁴ forms a 5-, 6-, or 7-membered heterocycloalkyl optionally substituted with one or more, also 1-3, unsubstituted C₁-C₄ alkyl; and wherein R⁶⁵ and R⁶⁶ are independently H, unsubstituted C₁-C₆ alkyl, or unsubstituted C₃-C₆ cycloalkyl.

In one example, regarding embodiments of Formula (I), (II), (III), (IV), (V), (VI), (VII), (VIII), (VIIIa), (VIIIb), (X), (Xa), (Xb), (XI), (XIa), (XIb), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), or (XIVf), R⁴ is selected from the group consisting of —NR⁶⁵R⁶⁶, C₁-C₆ alkyl optionally substituted with one or more, also 1-5, also 1-3, independently selected substituents R⁵⁹, 3- to 8-membered heteroalkyl optionally substituted with one or more, also 1-3, independently selected substituents R⁵⁹, C₃-C₆ cycloalkyl optionally substituted with one or more, also 1-3, independently selected substituents R⁶⁰, 3- to 6-membered heterocycloalkyl optionally substituted with one or more, also 1-3, independently selected substituents R⁶⁰, phenyl optionally substituted with one or more, also 1-3, independently selected substituents R⁶⁰, and 5 or 6 membered heteroaryl optionally substituted with one or more, also 1-3, independently selected substituents R⁶⁰; or R⁴ and R³, together with the atoms to which they are attached, are joined to form a 5-, 6-, or 7-membered heterocyclic ring optionally substituted with one or more, also 1-3, independently selected substituents R⁶⁰; wherein R⁵⁹ at each occurrence is independently —OR⁶¹; —NHR⁶¹, —NHR⁶¹R⁶², -halogen, —CN, —OH, or —NH₂; R⁶⁰ at each occurrence is independently —R⁵⁹ or —R⁶¹; R⁶¹ and R⁶² are independently unsubstituted C₃-C₆ cycloalkyl or C₁-C₄ alkyl optionally substituted with one or more, also 1-5, also 1-3, substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁶³R⁶⁴; or —NR⁶¹R⁶² forms a 5-, 6-, or 7-membered heterocycloalkyl optionally substituted with one or more, also 1-3, unsubstituted C₁-C₄ alkyl; wherein —NR⁶³R⁶⁴ forms a 5-, 6-, or 7-membered heterocycloalkyl optionally substituted with one or more, also 1-3, unsubstituted C₁-C₄ alkyl; and wherein R⁶⁵ and R⁶⁶ are independently H, unsubstituted C₁-C₆ alkyl, or unsubstituted C₃-C₆ cycloalkyl.

In one example, regarding embodiments of Formula (I), (II), (III), (IV), (V), (VI), (VII), (VIII), (VIIIa), (VIIIb), (X), (Xa), (Xb), (XI), (XIa), (XIb), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), or (XIVf), R⁴ is selected from the group consisting of —NR⁶⁵R⁶⁶, unsubstituted C₁-C₄ alkyl, C₁-C₄ haloalkyl, C₃-C₆ cycloalkyl optionally substituted with 1-2 substituents independently selected from the group consisting of —F, unsubstituted C₁-C₃ alkyl, and C₁-C₃ haloalkyl, 4- to 6-membered heterocycloalkyl optionally substituted with 1-2 substituents independently selected from the group consisting of —F, unsubstituted C₁-C₃ alkyl, and C₁-C₃ haloalkyl, phenyl optionally substituted with 1-3 substituents independently selected from the group consisting of —F, —Cl, —CN, unsubstituted C₁-C₄ alkyl, C₁-C₄ haloalkyl, unsubstituted C₁-C₄ alkoxy, and C₁-C₄ haloalkoxy, and 5 or 6 membered heteroaryl optionally substituted with 1-3 substituents independently selected from the group consisting of —F, —Cl, —CN, unsubstituted C₁-C₄ alkyl, C₁-C₄ haloalkyl, unsubstituted C₁-C₄ alkoxy, C₁-C₄ haloalkoxy; or R⁴ and R³, together with the atoms to which they are attached, are joined to form a 5-, 6-, or 7-membered heterocyclic ring optionally substituted with 1-2 substituents independently selected from the group consisting of fluoro, unsubstituted C₃-C₆ cycloalkyl, unsubstituted C₁-C₄ alkyl, and C₁-C₄ haloalkyl. In one example, R⁴ is selected from the group consisting of —NR⁶⁵R⁶⁶, unsubstituted C₁-C₄ alkyl, C₁-C₄ haloalkyl, C₃-C₆ cycloalkyl optionally substituted with 1-2 fluoro, 4- to 6-membered unsubstituted heterocycloalkyl, phenyl optionally substituted with 1-3 substituents independently selected from the group consisting of —F, —Cl, —CN, unsubstituted C₁-C₄ alkyl, C₁-C₄ haloalkyl, unsubstituted C₁-C₄ alkoxy, and C₁-C₄ haloalkoxy, and 5 or 6 membered heteroaryl optionally substituted with 1-3 substituents independently selected from the group consisting of —F, —Cl, —CN, unsubstituted C₁-C₄ alkyl, C₁-C₄ haloalkyl, unsubstituted C₁-C₄ alkoxy, C₁-C₄ haloalkoxy; or R⁴ and R³, together with the atoms to which they are attached, are joined to form a 5-, 6-, or 7-membered heterocyclic ring optionally substituted with 1-2 substituents independently selected from the group consisting of fluoro, unsubstituted C₃-C₆ cycloalkyl, unsubstituted C₁-C₄ alkyl, and C₁-C₄ haloalkyl.

In one example, regarding embodiments of Formula (I), (II), (III), (IV), (V), (VI), (VII), (VIII), (VIII a), (VIIIb), (X), (Xa), (Xb), (XI), (XIa), (XIb), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), or (XIVf), R⁴ is selected from the group consisting of —NH₂, unsubstituted C₁-C₃ alkyl, C₁-C₃ haloalkyl, C₃-C₆ cycloalkyl optionally substituted with 1-2 fluoro, 4- to 6-membered unsubstitutede heterocycloalkyl, phenyl optionally substituted with 1-3 substituents independently selected from the group consisting of —F, —Cl, —CN, unsubstituted C₁-C₄ alkyl, C₁-C₄ haloalkyl, unsubstituted C₁-C₄ alkoxy, and C₁-C₄ haloalkoxy, and 5 or 6 membered heteroaryl optionally substituted with 1-3 substituents independently selected from the group consisting of —F, —Cl, —CN, unsubstituted C₁-C₄ alkyl, C₁-C₄ haloalkyl, unsubstituted C₁-C₄ alkoxy, C₁-C₄ haloalkoxy; or R⁴ and R³, together with the atoms to which they are attached, are joined to form a 5-, 6-, or 7-membered heterocyclic ring optionally substituted with 1-2 substituents independently selected from the group consisting of fluoro, unsubstituted C₃-C₆ cycloalkyl, unsubstituted C₁-C₄ alkyl, and C₁-C₄ haloalkyl, preferably wherein R⁴ as C₃-C₆ cycloalkyl is cyclopropyl, cyclobutyl or cyclopentyl, each optionally substituted with 1-2 fluoro, and R⁴ as 4- to 6-membered unsubstituted heterocycloalkyl is oxetane, tetrahydrofuran or tetrahydropyran, and R⁴ as 5 or 6 membered heteroaryl is pyridiyl, pyridimidinyl, pyrazolyl, isothiazolyl, isoxazolyl, imidazolyl, thiazolyl, or oxazolyl, each optionally substituted with 1-3 substituents independently selected from the group consisting of —F, —Cl, —CN, unsubstituted C₁-C₄ alkyl, C₁-C₄ haloalkyl, unsubstituted C₁-C₄ alkoxy, C₁-C₄ haloalkoxy.

In one example of Formula (IX), (IXa), (IXb), (XVa), (XVb), (XVc), (XVd), (XVe), or (XVf), q is 1 or 2, Z is C(R²⁴)₂ and each R²⁴ is independently H, fluoro, unsubstituted C₁-C₄ alkyl, or C₁-C₄ haloalkyl. In one example, q is 1 or 2, Z is C(R²⁴)₂ and each R²⁴ is independently H, fluoro, unsubstituted C₁-C₂ alkyl, or C₁-C₂ haloalkyl. In one example, q is 1 or 2, Z is C(R²⁴)₂ and each R²⁴ is H. In one example, q is 2, Z is 0 and each R²⁴ is independently H, fluoro, unsubstituted C₁-C₄ alkyl, or C₁-C₄ haloalkyl. In one example, q is 2, Z is 0 and each R²⁴ is independently H, fluoro, unsubstituted C₁-C₂ alkyl, or C₁-C₂ haloalkyl. In one example, q is 2, Z is 0 and each R²⁴ is H. In one example, q is 2, Z is N(R⁶⁷), and each R²⁴ is independently H, fluoro, unsubstituted C₁-C₄ alkyl, or C₁-C₄ haloalkyl. In one example, q is 2, Z is N(R⁶⁷), R⁶⁷ is H, unsubstituted C₃-C₆ cycloalkyl or unsubstituted C₁-C₄ alkyl, and each R²⁴ is independently H, fluoro, unsubstituted C₁-C₂ alkyl, or C₁-C₂ haloalkyl. In one example, q is 2, Z is N(R⁶⁷), R⁶⁷ is H or unsubstituted C₁-C₂ alkyl, and each R²⁴ is H.

In one example, regarding embodiments of Formula (I), (II), (III), (IV), (V), (VI), (VII), (VIII), (VIIIa), (VIIIb), (X), (Xa), (Xb), (XI), (XIa), (XIb), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), or (XIVf), U¹ is N, U² is N, U³ is CH, R² is H, R³ is ethyl, and R⁴ is cyclobutyl.

In one example, a compound according to Formula (XIVa) is provided wherein

U¹ is N, U² is N, R^{1b} is H, R³ is ethyl, and R⁴ is cyclobutyl, preferably wherein R⁶ is phenyl optionally substituted with 1-2 substituents independently selected from the group consisting of —F, —Cl, —Br, —CN, —CF₃, and —OCF₃.

In one example, regarding embodiments of Formula (I), (II), (III), (IV), (V), (VI), (VII), (VIII), (VIII a), (VIIIb), (XI), (XIa), (XIb), (XIIa), (XIIb), (XIIc), (XIId), (XIIe), (XIIf), (XIIIa), (XIIIb), (XIIIc), (XIIId), (XIIIe), (XIIIf), (XIVa), (XIVb), (XIVc), (XIVd), (XIVe), or (XIVf), R⁴ is preferably other than benzyl. In a further example according to any of the embodiments of Formula (I), R⁴ is preferably other than halogen-substituted benzyl. In a particular example, R⁴ is preferably other than:

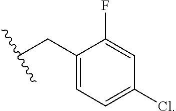

In one embodiment, compounds are provided having a structure according to

Formula (XVI):

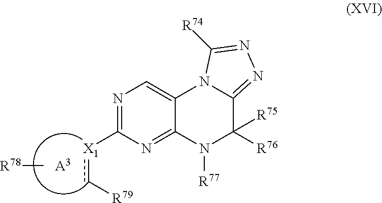

or a salt or solvate thereof, wherein:
X₁ is C or N and the dashed line represents a single or double bond;
A³ is a ring selected from the group consisting of phenyl, pyridine, pyrimidine, pyrazine, pyridazine, pyrrole, pyrazole, imidazole, thiazole, isothiazole, isoxazole, triazole, thiadiazole, benzimidazole, indole, pyrrolo[2,3-b]pyridine, quinoline, pyrrolidine, piperidine, piperazine, and dihydro-imidazole;
R⁷⁴ is hydrogen or methyl;
R⁷⁵ is hydrogen, methyl (e.g. —CD₃ or —CH₃), ethyl (e.g. —CD₂CD₃ or —CH₂CH₃), —CH₂-cyclopropyl, or —CH₂CF₃;
R⁷⁶ is methyl (e.g. —CD₃ or —CH₃), ethyl (e.g. —CD₂CD₃ or —CH₂CH₃), —CH₂-cyclopropyl, or —CH₂CF₃;
or R⁷⁵ and R⁷⁶, together with the carbon atom to which they are attached, are optionally joined to form cyclobutyl;
R⁷⁷ is selected from the group consisting of —NH₂, —NHCH₃, —NHcyclopropyl, pyrrolidine, —CH₂-cyclopropyl, —CH(CH₃)-cyclopropyl, cyclopropyl, cyclobutyl optionally substituted with 1 or 2 fluoro, cyclopentyl optionally substituted with 1 or 2 fluoro, isopropyl (e.g. —CH(CH₃)₂ or —CD(CD₃)₂), —CH₂CH₂CF₃, tetrahydropyran, tetrahydrofuran, oxetane, phenyl optionally substituted with 1 or 2 substituents R⁸⁰, pyrazole optionally substituted with 1 substituent R⁸¹, and pyrimidine;
or R⁷⁷ and R⁷⁶, together with the atoms to which they are attached, are optionally joined to form a substituted or unsubstituted 5- to 7-membered heterocyclic ring selected from the group consisting of

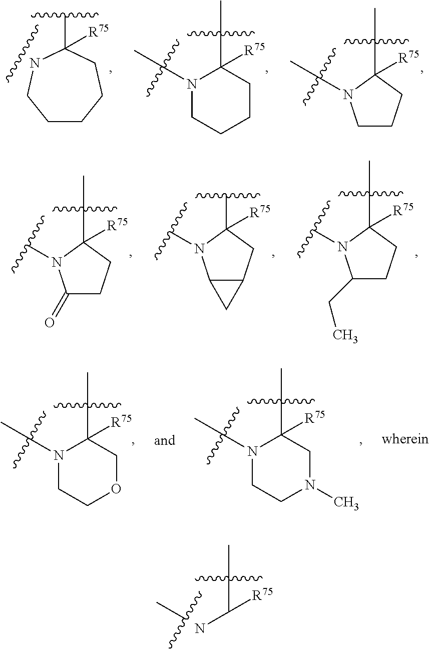

represents the core ring of Formula I, i.e. the N attached to R⁷⁷ and the C attached to R⁷⁶;
or R⁷⁷, R⁷⁵ and R⁷⁶, together with the atoms to which they are attached, are optionally joined to form a substituted or unsubstituted 7-membered heterocyclic ring selected from the group consisting of

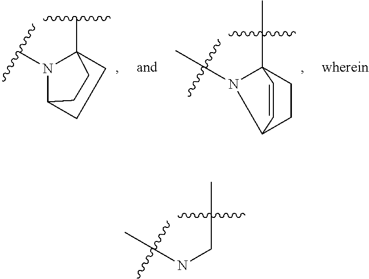

represents the core ring of Formula I, i.e. the N attached to R⁷⁷ and the C attached to R⁷⁶/R⁷⁵;
R⁷⁸ is hydrogen, —Br, —CN, —CH₃, —CH₂CN, —CH₂CH₂NH₂, —OH, —O⁻, ═O, —OCH₃, —Obenzyl, —C(O)OH, —C(O)OCH₃, —C(O)OCH₂CH₃, —C(O)NH₂, —C(O)NHCH₃, —C(O)N(CH₃)₂,

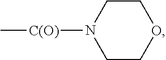

—NH₂, ═NH, —NHCH₃, —N(CH₃)₂, —NHS(O)₂CH₃, —S(O)₂CH₃, phenyl, thiazole, pyridine or pyrazine;
R⁷⁹ is hydrogen, —Cl, —Br, —CH₃, —CF₃, —CH₂NH₂, —NH₂, —CH₂NHC(O)OCH₃, —CH₂NHC(O)CH₃, —CH₂NHC(O)phenyl, —CH₂NHS(O)₂CH₃, —CH₂NHS(O)₂phenyl, —NHC(O)CH₃, —NHC(O)OCH₃, —NHC(O)phenyl, —NHS(O)₂CH₃, —NHS(O)₂phenyl, —CH≡CHphenyl, cyclopropyl, cyclopentenyl, benzyl, phenyl optionally sub with 1, 2 or 3 substituents R⁸², pyridine optionally substituted with 1 fluoro, pyrimidine, pyrazine, pyridazine, pyrazole, thiazole, oxazole, thiophene optionally substituted with 1 chloro, pyrrolidine, oxazolidinone, pyrrolidinone, dihydropyran, tetrahydropyran, morpholine, 4-methyl-piperazine, pyrrolidine-dione, pyridinone, isoquinoline, or quinoline;
R⁸⁰ at each occurrence is independently —C(O)NH₂, fluoro, chloro, cyano, pyrazole, triazole, pyridine or pyrimidine;
R⁸¹ is methyl or 2-(trimethylsilyl)ethoxy)methyl, cyclopropyl, or —CH₂-cyclopropyl; and
R⁸² at each occurrence is independently selected from the group consisting of fluoro, chloro, bromo, —S(O)₂CH₃, —OCF₃, —CF₃, —CN, pyridine, triazole, and pyrazole.

In one embodiment, compounds are provided having a structure according to Formula (XVI), or a salt or solvate thereof, wherein:
A³ is a ring selected from the group consisting of phenyl, pyridin-2-yl, pyridin-3-yl, pyridin-4-yl, pyrimidin-5-yl, pyrazin-2-yl, pyridazin-4-yl, pyridin-2-on-4-yl, pyridin-4-imine, pyrrol-2-yl, pyrazol-1-yl, pyrazol-3-yl, pyrazol-4-yl, imidazol-1-yl, thiazol-5-yl, isothiazol-4-yl, isoxazol-4-yl, 1,2,3-triazol-5-yl, 1,2,4-triazol-1-yl, 1,2,3-thiadiazol-5-yl, benzimidazol-1-yl, indol-1-yl, indol-2-yl, indol-7-yl, pyrrolo[2,3-b]pyridin-5-yl, quinolin-8-yl, pyrrolidin-1-yl, piperidin-1-yl, piperazin-1-yl, and 4,5-dihydro-1H-imidazol-1-yl(A³ orientation is preferably structurally as follows:

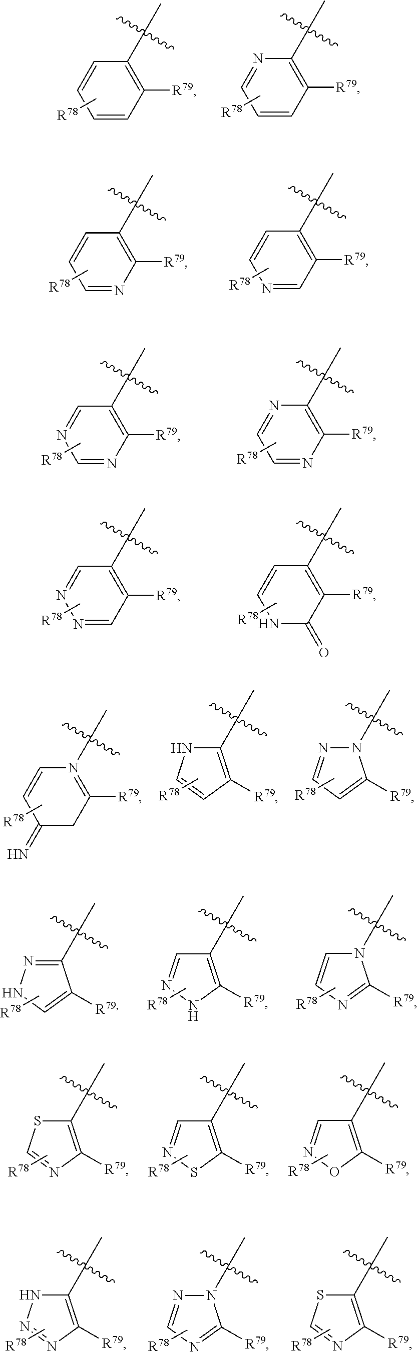

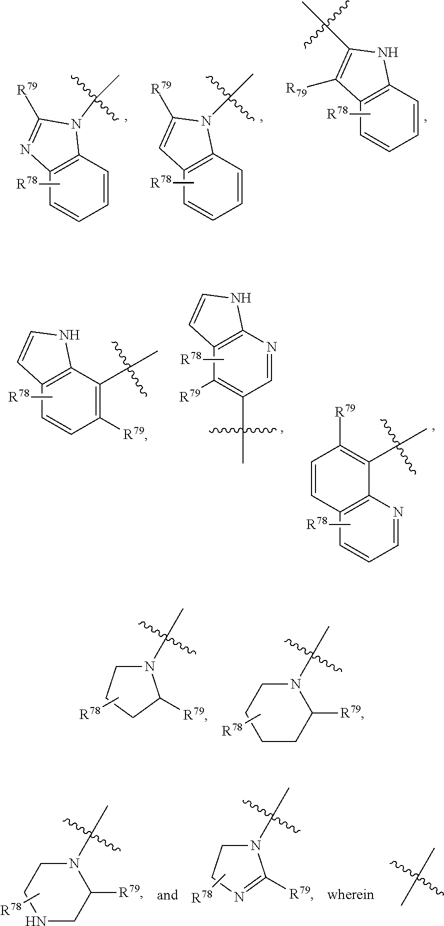

represents the attachment of X₁ to the 7-position of the 4,5-dihydro-[1,2,4]triazolo[4,3-f]pteridine core);
R⁷⁴ is hydrogen or —CH₃;
R⁷⁵ is hydrogen, —CD₃, —CH₃, —CD₂CD₃, —CH₂CH₃, —CH₂-cyclopropyl, or —CH₂CF₃;
R⁷⁶ is —CD₃, —CH₃, —CD₂CD₃, —CH₂CH₃, —CH₂-cyclopropyl, or —CH₂CF₃;
or R⁷⁵ and R⁷⁶, together with the carbon atom to which they are attached, are optionally joined to form cyclobutyl;
R⁷⁷ is selected from the group consisting of —NH₂, —NHCH₃, —NHcyclopropyl, pyrrolidin-1-yl, —CH₂-cyclopropyl, —CH(CH₃)-cyclopropyl, cyclopropyl, cyclobutyl, 3-fluorocyclobutyl, 3,3-difluorocyclobutyl, cyclopentyl, 3,3-difluorocyclopentyl, —CH(CH₃)₂, —CD(CD₃)₂, —CH₂CH₂CF₃, tetrahydro-2H-pyran-4-yl, tetrahydrofuran-3-yl, oxetan-3-yl, phenyl, 4-fluoro-phenyl, 4-chloro-phenyl, 3-cyano-phenyl, 4-cyano-phenyl, 3-pyrimidin-5-yl-phenyl, 3-pyrazol-1-yl-phenyl, 3-pyridin-3-yl-phenyl, 3-1,2,4-triazol-1-yl-phenyl, pyrazol-3-yl, pyrazol-4-yl, 1-methyl-pyrazol-4-yl, 1-cyclopropyl-pyrazol-4-yl, 1-cyclopropylmethyl-pyrazol-4-yl, 1-(2-(trimethylsilyl)ethoxy)methyl)-pyrazol-4-yl, and pyrimidin-5-yl;
or R⁷⁷ and R⁷⁶, together with the atoms to which they are attached, are optionally joined to form a substituted or unsubstituted 5- to 7-membered heterocyclic ring selected from the group consisting of

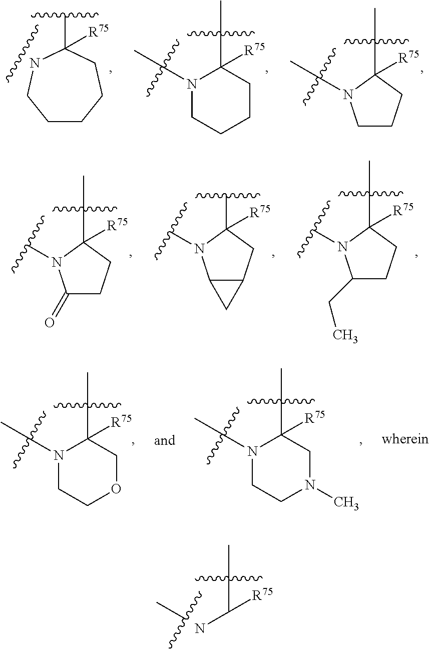

represents the core ring of Formula I, i.e. the N attached to R⁷⁷ and the C attached to R⁷⁶;
or R⁷⁷, R⁷⁵ and R⁷⁶, together with the atoms to which they are attached, are optionally joined to form a substituted or unsubstituted 7-membered heterocyclic ring selected from the group consisting of

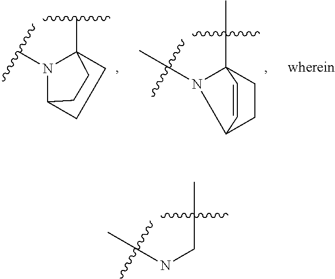

represents the core ring of Formula I, i.e. the N attached to R⁷⁷ and the C attached to R⁷⁶/R⁷⁵;
R⁷⁸ is hydrogen, —Br, —CN, —CH₃, —CH₂CN, —CH₂CH₂NH₂, —OH, ═O, —O⁻, —OCH₃, —Obenzyl, —C(O)OH, —C(O)OCH₃, —C(O)OCH₂CH₃, —C(O)NH₂, —C(O)NHCH₃, —C(O)N(CH₃)₂,

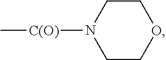

—NH₂, —NHCH₃, —N(CH₃)₂, —NHS(O)₂CH₃, —S(O)₂CH₃, phenyl, thiazol-2-yl, thiazol-4-yl, pyridin-3-yl, and pyrazin-2-yl;
R⁷⁹ is hydrogen, —Cl, —Br, —CH₃, —CF₃, —CH₂NH₂, —NH₂, —CH₂NHC(O)OCH₃, —CH₂NHC(O)CH₃, —CH₂NHC(O)phenyl, —CH₂NHS(O)₂CH₃, —CH₂NHS(O)₂-phenyl, —NHC(O)CH₃, —NHC(O)OCH₃, —NHC(O)phenyl, —NHS(O)₂CH₃, —NHS(O)₂-phenyl, —CH≡CHphenyl, cyclopropyl, cyclopent-1-enyl, benzyl, phenyl optionally substituted with 1, 2, or 3 substituents R⁸², pyridin-2-yl, 5-fluoro-pyridin-2-yl, pyridin-3-yl, 5-fluoro-pyridin-3-yl, pyridin-4-yl, pyrimidin-2-yl, pyrimidin-5-yl, pyrazin-2-yl, pyridazin-3-yl, pyrazol-1-yl, pyrazol-5-yl, pyrazol-4-yl, thiazol-2-yl, thiazol-4-yl, oxazol-2-yl, 5-C1-thiophen-2-yl, pyrrolidin-1-yl, oxazolidin-2-on-3-yl, 2-oxopyrrolidin-1-yl, 3,6-dihydro-2H-pyran-4-yl, tetrahydro-2H-pyran-4-yl, morpholin-4-yl, 4-methyl-piperazin-1-yl, pyrrolidine-2,5-dion-1-yl, pyridin-2-on-1-yl, isoquinolin-1-yl, quinolin-5-yl, and quinolin-3-yl; and
R⁸² gives substitution of the phenyl ring selected from the group consisting of 4-S(O)₂CH₃, 3-OCF₃, 4-OCF₃, 3-CF₃, 4-CF₃, 2-F, 3-F, 3-Cl, 3-Br, 4-F, 2,3-diF, 2,4-diF, 2-Cl-4-F, 3,4-diF, 3,5-diCl, 3,5-diF, 3-F-5-CF₃, 3-C₁₋₄-F, 3-CN, 4-CN, 3,4,5-triF, 3-pyridin-3-yl, 3-1,2,4-triazol-1-yl, and 3-pyrazol-1-yl.

In one embodiment, compounds are provided having a structure according to Formula (XVI), or a salt or solvate thereof, wherein:
A³ is a ring selected from the group consisting of phenyl, pyridin-2-yl, pyridin-3-yl, pyridin-4-yl, pyrimidin-5-yl, pyrazin-2-yl, pyridin-2-one, pyridin-4-imine, pyrazol-1-yl, pyrazol-4-yl, imidazol-1-yl, thiazol-5-yl, 1,2,3-triazol-5-yl, 1,2,4-triazol-1-yl, 1,2,3-thiadiazol-5-yl, indol-1-yl, indol-2-yl, indol-7-yl, piperazin-1-yl, 4,5-dihydro-1H-imidazol-1-yl;
R⁷⁴ is hydrogen or —CH₃;
R⁷⁵ is hydrogen, —CD₃, —CH₃, —CD₂CD₃, —CH₂CH₃ or —CH₂CF₃;
R⁷⁶ is —CD₃, —CH₃, —CD₂CD₃, —CH₂CH₃, or —CH₂CF₃;
or R⁷⁵ and R⁷⁶, together with the carbon atom to which they are attached, are optionally joined to form cyclobutyl;
R⁷⁷ is selected from the group consisting of —NH₂, cyclopropyl, cyclobutyl, 3,3-difluorocyclobutyl, cyclopentyl, —CH(CH₃)₂, —CD(CD₃)₂, —CH₂CH₂CF₃, tetrahydro-2H-pyran-4-yl, tetrahydrofuran-3-yl, oxetan-3-yl, phenyl, 4-fluoro-phenyl, 4-chloro-phenyl, 3-cyano-phenyl, 4-cyano-phenyl, pyrazol-3-yl, pyrazol-4-yl, 1-methyl-pyrazol-4-yl, and pyrimidin-5-yl;
or R⁷⁷ and R⁷⁶, together with the atoms to which they are attached, are optionally joined to form a substituted or unsubstituted 5- to 6-membered heterocyclic ring selected from the group consisting of

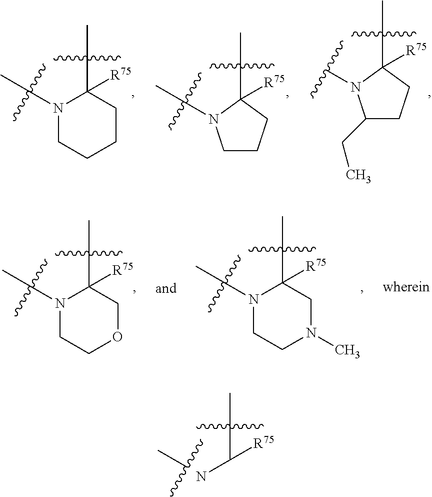

represents the core ring of Formula I, i.e. the N attached to R⁷⁷ and the C attached to R⁷⁶;
R⁷⁸ is hydrogen, —CN, —Br, —CH₃, —CH₂CN, —CH₂CH₂NH₂, —OH, ═O, —O⁻, —C(O)OH, —C(O)OCH₃, —C(O)OCH₂CH₃, —C(O)NH₂, —C(O)NHCH₃, —C(O)N(CH₃)₂,

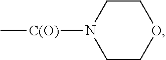

—NH₂, —N(CH₃)₂, —NHS(O)₂CH₃, phenyl, thiazol-2-yl, thiazol-4-yl, or pyridin-3-yl;
R⁷⁹ is hydrogen, —Cl, —CH₃, —NH₂, —CH₂NHC(O)OCH₃, —CH₂NHC(O)CH₃, —CH₂NHS(O)₂CH₃, —NHC(O)CH₃, —NHC(O)OCH₃, —NHS(O)₂CH₃, cyclopropyl, cyclopent-1-enyl, phenyl optionally substituted with 1, 2, or 3 substituents R⁸², pyridin-2-yl, 5-fluoro-pyridin-2-yl, pyridin-3-yl, pyridin-4-yl, pyrimidin-2-yl, pyrimidin-5-yl, pyrazin-2-yl, pyridazin-3-yl, pyrazol-1-yl, pyrazol-5-yl, pyrazol-4-yl, thiazol-2-yl, thiazol-4-yl, oxazol-2-yl, pyrrolidin-1-yl, oxazolidin-2-on-3-yl, 2-oxopyrrolidin-1-yl, tetrahydro-2H-pyran-4-yl, morpholin-4-yl, 4-methyl-piperazin-1-yl, quinolin-5-yl, or quinolin-3-yl; and
R⁸² gives substitution of the phenyl ring selected from the group consisting of 4-S(O)₂CH₃, 4-CF₃, 3-F, 3-Cl, 3-Br, 4-F, 2,4-diF, 3,4-diF, 3,5-diF, 3-C₁₋₄-F, 4-CN, 3-1,2,4-triazol-1-yl, and 3-pyrazol-1-yl.

In one embodiment, compounds are provided having a structure according to Formula (XVI), or a salt or solvate thereof, wherein:
A³ is a ring selected from the group consisting of pyridin-3-yl, pyridin-4-yl, pyridin-2-one, pyridin-4-imine, pyrazol-1-yl, pyrazol-4-yl, imidazol-1-yl, thiazol-5-yl, 1,2,4-triazol-1-yl, and 1,2,3-thiadiazol-5-yl;
R⁷⁴ is hydrogen;
R⁷⁵ is hydrogen, —CD₃, —CH₃, —CD₂CD₃, or —CH₂CH₃;
R⁷⁶ is —CD₃, —CH₃, —CD₂CD₃, or —CH₂CH₃;
R⁷⁷ is selected from the group consisting of —NH₂, cyclopropyl, cyclobutyl, cyclopentyl, —CH(CH₃)₂, —CD(CD₃)₂, tetrahydro-2H-pyran-4-yl, tetrahydrofuran-3-yl, oxetan-3-yl, 4-chloro-phenyl, 4-cyano-phenyl, pyrazol-3-yl, pyrazol-4-yl, 1-methyl-pyrazol-4-yl, and pyrimidin-5-yl;
or R⁷⁷ and R⁷⁶, together with the atoms to which they are attached, are optionally joined to form a substituted or unsubstituted 5- to 6-membered heterocyclic ring selected from the group consisting of

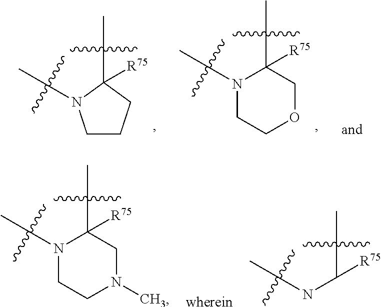

represents the core ring of Formula I, i.e. the N attached to R⁷⁷ and the C attached to R⁷⁶;
R⁷⁸ is hydrogen, —CH₃, —CH₂CH₂NH₂, —OH, —O⁻, —C(O)OH, —C(O)OCH₂CH₃, —C(O)NH₂, —C(O)NHCH₃, —C(O)N(CH₃)₂,

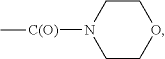

—NHCH₃, or pyridin-3-yl; and
R⁷⁹ is hydrogen, phenyl, 4-methylsulfonyl-phenyl, 4-fluoro-phenyl, 2,3-difluoro-phenyl, 2,4-difluoro-phenyl, pyridin-2-yl, 5-fluoro-pyridin-2-yl, pyridin-3-yl, pyridin-4-yl, pyrimidin-2-yl, pyrimidin-5-yl, pyrazin-2-yl, pyridazin-3-yl, pyrazol-5-yl, pyrazol-4-yl, thiazol-2-yl, oxazol-2-yl, or oxazolidin-2-on-3-yl.

In one embodiment, for compounds of Formula (XVI), R⁷⁵, R⁷⁶, and R⁷⁷ are selected to give a structure selected from the group consisting of Formula (XVIa), Formula (XVIb), Formula (XVIc), Formula (XVId), and Formula (XVIe), as follows:

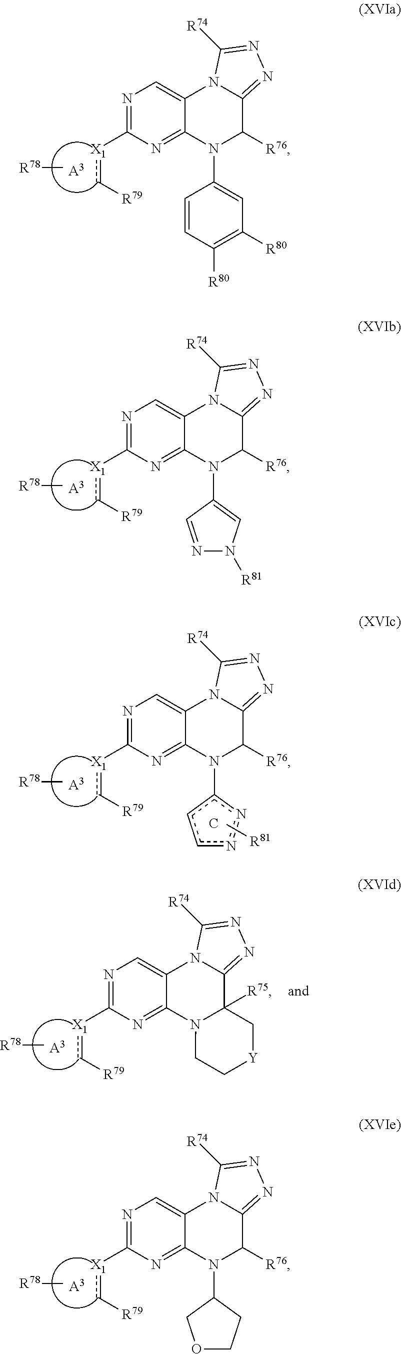

or a salt or solvate thereof, wherein:
C is pyrazole, wherein R⁸¹ is bound to either of the nitrogens in the pyrazole ring;
Y is O or N—CH₃; and
X1, A³, R⁷⁴, R⁷⁵, R⁷⁶, R⁷⁸, R⁷⁹, R⁸⁰ and R⁸¹ are as defined for Formula XVI.

In one embodiment, for compounds of Formula (XVI), the preferred stereoisomer at the carbon bound to R⁷⁵ and R⁷⁶ is as follows:

when R⁷⁵ is H and R⁷⁶ is —CD₃, —CH₃, —CD₂CD₃, —CH₂CH₃, —CH₂-cyclopropyl, or —CH₂CF₃, preferably, —CD₂CD₃, —CH₂CH₃, or —CH₂CF₃, the preferred isomer is represented by the following structure of Formula (XVIf):

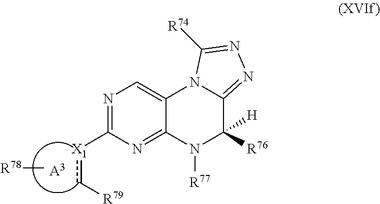

and when R⁷⁵ is —CD₂CD₃, —CH₂CH₃, or —CH₂CF₃, and R⁷⁶ and R⁷⁷, together with the atoms to which they are attached, combine to form a substituted or unsubstituted 3- to 8-membered heterocyclic ring, the preferred isomer is represented by the following structure of Formula (XVIg), where the dotted line connecting R⁷⁶ and R⁷⁷ represents one of the rings as provided in Formula (XVI) above:

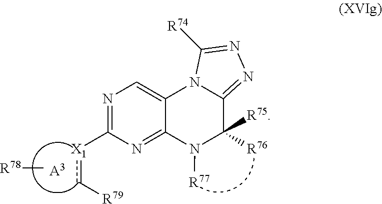

In one embodiment, compounds are provided having a structure selected from the group consisting of Formula (XVIIa), Formula (XVIIb), Formula (XVIIc), Formula (XVIId), and Formula (XVIIe), as follows:

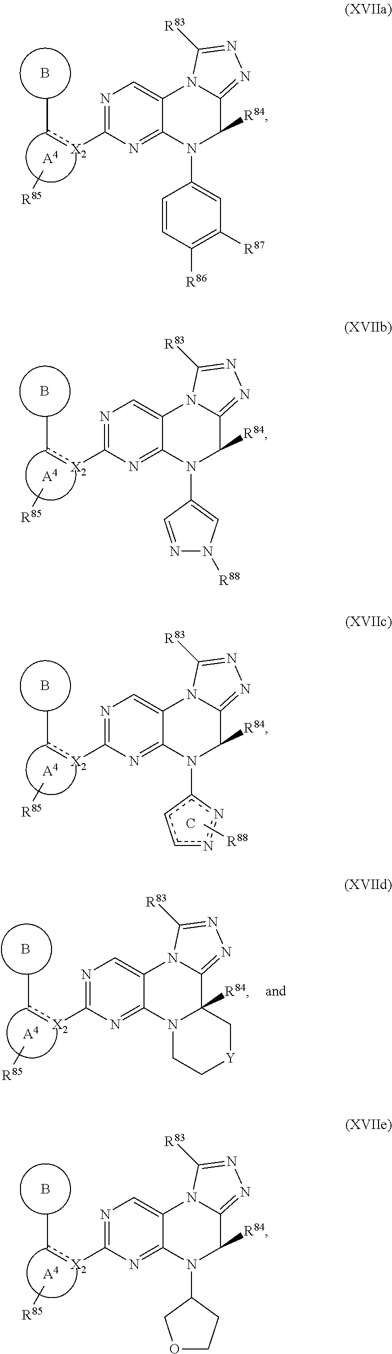

or a salt or solvate thereof, wherein:
X₂ is C or N and the dashed line represents a single or double bond;
Y is O or N—CH₃;
A⁴ is selected from the group consisting of phenyl, pyridin-2-yl, pyridin-3-yl, pyridin-4-yl, pyrimidin-5-yl, pyrazin-2-yl, pyridin-2-one, pyridin-4-imine, pyrazol-1-yl, pyrazol-4-yl, imidazol-1-yl, thiazol-5-yl, isothiazol-4-yl, isoxazol-4-yl, 1,2,3-triazol-5-yl, 1,2,4-triazol-1-yl, 1,2,3-thiadiazol-5-yl, indol-1-yl, indol-2-yl, indol-7-yl, piperazin-1-yl, 4,5-dihydro-1H-imidazol-1-yl;
B is selected from the group consisting of phenyl optionally substituted with 1, 2, or 3 substituents R⁸⁹, pyridin-2-yl, 5-fluoro-pyridin-2-yl, pyridin-3-yl, pyridin-4-yl, pyrimidin-2-yl, pyrimidin-5-yl, pyrazin-2-yl, pyridazin-3-yl, pyrazol-1-yl, pyrazol-5-yl, pyrazol-4-yl, thiazol-2-yl, thiazol-4-yl, oxazol-2-yl, pyrrolidin-1-yl, oxazolidin-2-on-3-yl, 2-oxopyrrolidin-1-yl, tetrahydro-2H-pyran-4-yl, morpholin-4-yl, 4-methyl-piperazin-1-yl, quinolin-5-yl, and quinolin-3-yl;
C is pyrazole, wherein R⁸⁸ is bound to either of the nitrogens in the pyrazole ring;
R⁸³ is hydrogen or —CH₃;
R⁸⁴ is —CD₂CD₃ or —CH₂CH₃;
R⁸⁵ is hydrogen, —CH₃, —Br, —CN, or —NH₂;
R⁸⁶ is hydrogen, —F, —Cl, —C(O)NH₂, or —CN;
R⁸⁷ is hydrogen, —F, —Cl, —C(O)NH₂, or —CN;
R⁸⁸ is hydrogen, methyl, cyclopropyl, or —CH₂-cyclopropyl; and
R⁸⁹ at each occurrence is independently selected from the group consisting of fluoro, chloro, bromo, —S(O)₂CH₃, —OCF₃, —CF₃, —CN, pyridine, triazole, and pyrazole.

In one embodiment, compounds are provided having a structure selected from the group consisting of Formula (XVIIa), Formula (XVIIb), Formula (XVIIc), Formula (XVIId), and Formula (XVIIe), or a salt or solvate thereof, wherein:
X₂ is C or N and the dashed line represents a single or double bond;
Y is O or N—CH₃;
A⁴ is selected from the group consisting of pyridin-3-yl, pyridin-4-yl, pyridin-2-one, pyridin-4-imine, pyrazol-1-yl, pyrazol-4-yl, imidazol-1-yl, thiazol-5-yl, 1,2,4-triazol-1-yl, and 1,2,3-thiadiazol-5-yl;
B is selected from the group consisting of phenyl, 4-methylsulfonyl-phenyl, 4-fluoro-phenyl, 2,3-difluoro-phenyl, 2,4-difluoro-phenyl, pyridin-2-yl, 5-fluoro-pyridin-2-yl, pyridin-3-yl, pyridin-4-yl, pyrimidin-2-yl, pyrimidin-5-yl, pyrazin-2-yl, pyridazin-3-yl, pyrazol-5-yl, pyrazol-4-yl, thiazol-2-yl, oxazol-2-yl, or oxazolidin-2-on-3-yl;
R⁸³ is hydrogen or —CH₃;
R⁸⁴ is —CD₂CD₃ or —CH₂CH₃;
R⁸⁵ is hydrogen, —CH₃, —Br, —CN, or —NH₂;
R⁸⁶ is hydrogen, —F, —Cl, —C(O)NH₂, or —CN;
R⁸⁷ is hydrogen, —F, —Cl, —C(O)NH₂, or —CN; and
R⁸⁸ is —CH₃, cyclopropyl, or —CH₂-cyclopropyl.

In one embodiment, compounds are provided having a structure selected from the group consisting of Formula (XVIIb), Formula (XVIIc), Formula (XVIId), and Formula (XVIIe), or a salt or solvate thereof, wherein:
X₂ is C or N and the dashed line represents a single or double bond;
Y is O or N—CH₃;
A⁴ is selected from the group consisting of pyridin-3-yl, pyridin-4-yl, pyrazol-4-yl, and imidazol-1-yl;
B is selected from the group consisting of phenyl, 4-fluoro-phenyl, 2,3-difluoro-phenyl, 2,4-difluoro-phenyl, 5-fluoro-pyridin-2-yl, and thiazol-2-yl;
R⁸³ is hydrogen;
R⁸⁴ is —CD₂CD₃ or —CH₂CH₃;
R⁸⁵ is hydrogen or —CH₃; and
R⁸⁸ is —CH₃.

Exemplary compounds as described herein, e.g. compounds of Formula (I), and their in vitro biological activities are listed in the table of Example A.

### In Vitro Activities

Certain compounds as described herein, e.g. compounds of Formula (I), exhibit various in vitro biological activities (see, e.g., Example A), such as activity against polo-like kinases (PLKs). In vitro assays for the determination of PLK activities are known in the art and exemplary assay formats are described herein (see e.g., Example A). Many compounds as described herein, e.g. compounds of Formula (I), are especially active against PLK2, but may also inhibit PLK1 and PLK3.

In one example, the compounds as described herein, e.g. compounds of Formula (I), are inhibitors of PLK2 with an IC₅₀ of less than about 50 μM, less than about 40 μM, less than about 30 μM, less than about 20 μM or less than about 10 μM. In another example, the compounds of Formula (I) exhibit inhibitory activity against PLK2 with an IC₅₀ of less than about 9 μM, less than about 8 μM, less than about 7 μM, less than about 6 μM, less than about 5 μM, less than about 4 μM, less than about 3 μM, less than about 2 μM, or less than about 1 μM. In yet another example, the compounds of Formula (I) exhibit inhibitory activity against PLK2 with an IC₅₀ of less than about 0.9 μM, less than about 0.8 μM, less than about 0.7 μM, less than about 0.6 μM, less than about 0.5 μM, less than about 0.4 μM, less than about 0.3 μM, less than about 0.2 μM. In a particular example, the compounds of Formula (I) exhibit inhibitory activity against PLK2 with an IC₅₀ of less than about 0.1 μM (100 nM). In another particular example, the compounds of Formula (I) exhibit inhibitory activity against PLK2 with an IC₅₀ of less than about 90 nM, less than about 80 nM, less than about 70 nM, less than about 60 nM, less than about 50 nM, less than about 40 nM, less than about 30 nM or less than about 20 nM. In another particular example, the compounds of Formula (I) exhibit inhibitory activity against PLK2 with an IC₅₀ of less than about 10 nM.

In one example, the compounds as described herein, e.g. compounds of Formula (I), are also inhibitors of PLK1 with an IC₅₀ of less than about 1 μM, less than about 0.9 μM, less than about 0.8 μM, less than about 0.7 μM, less than about 0.6 μM, less than about 0.5 μM, less than about 0.4 μM, less than about 0.3 μM, less than about 0.2 μM. In a particular example, the compounds of Formula (I) exhibit inhibitory activity against PLK1 with an IC₅₀ of less than about 0.1 μM (100 nM). In another particular example, the compounds of Formula (I) exhibit inhibitory activity against PLK1 with an IC₅₀ of less than about 90 nM, less than about 80 nM, less than about 70 nM, less than about 60 nM, less than about 50 nM, less than about 40 nM, less than about 30 nM or less than about 20 nM. In another particular example, the compounds of Formula (I) exhibit inhibitory activity against PLK1 with an IC₅₀ of less than about 10 nM.

In one example, compounds as described herein, e.g. compounds of Formula (I), inhibit PLK2 and are selective against certain other members of the PLK family. Particularly, compounds of Formula (I) inhibit PLK2 and are selective against PLK1 or PLK3. For the purpose of this application the selectivity of the instant compounds for PLK2 over other PLKs is expressed in a ratio of IC₅₀ values. Those can be determined using assays known in the art or those described herein (see e.g., Example A).

In one example, compounds as described herein, e.g. compounds of Formula (I), inhibit PLK2 and are selective against other kinases. Particularly, compounds of Formula (I) inhibit PLK2 and are selective against one or more kinases selected from the group consisting of CDK-1, CDK-2, CDK-5, CLK-1, CLK-2, CLK-3, CLK-4, NEK-1, NEK-2, NEK-4, NEK-6, NEK-7, MAP4K4 and STK16. In one example, compounds are selective against other kinases, such as one or more kinases selected from the group consisting of CDK-1, CDK-2, CDK-5, CLK-1, CLK-2, CLK-3, CLK-4, NEK-1, NEK-2, NEK-4, NEK-6, NEK-7, MAP4K4 and STK16, and are selective against other PLK family members, including PLK1 or PLK3. For the purpose of this application the selectivity of the instant compounds for PLK2 over other kinases is expressed in a ratio of IC₅₀ values, or in some instances as a ratio of % inhibition at a given concentration of compound, such as at 10 μM, which can be determined using assays known in the art or those described herein (see e.g., Example A).

Certain compounds as described herein are characterized by the following inhibitory activities involving PLK2 and PLK1. In one example, the ratio of IC₅₀ (PLK2)/IC₅₀ (PLK1) is less than about 1, less than about 0.9, less than about 0.8, less than about 0.7, less than about 0.6, less than about 0.5, less than about 0.4, less than about 0.3, less than about 0.2 or less than about 0.1. In another example, the ratio of IC₅₀ (PLK2)/IC₅₀ (PLK1) is less than about 0.09, less than about 0.08, less than about 0.07, less than about 0.06, less than about 0.05, less than about 0.04, less than about 0.03, less than about 0.02 or less than about 0.01. In a further example, the ratio of IC₅₀ (PLK2)/IC₅₀ (PLK1) is less than about 0.009, less than about 0.008, less than about 0.007, less than about 0.006, less than about 0.005, less than about 0.004, less than about 0.003, less than about 0.002 or less than about 0.001. In yet another example, the ratio of IC₅₀ (PLK2)/IC₅₀ (PLK1) is less than about 0.0009, less than about 0.0008, less than about 0.0007, less than about 0.0006, less than about 0.0005, less than about 0.0004, less than about 0.0003, less than about 0.0002 or less than about 0.0001.

Certain compounds as described herein are characterized by the following inhibitory activities involving PLK2 and PLK3. In one example, the ratio of IC₅₀ (PLK2)/IC₅₀ (PLK3) is less than about 1, less than about 0.9, less than about 0.8, less than about 0.7, less than about 0.6, less than about 0.5, less than about 0.4, less than about 0.3, less than about 0.2 or less than about 0.1. In another example, the ratio of IC₅₀ (PLK2)/IC₅₀ (PLK3) is less than about 0.09, less than about 0.08, less than about 0.07, less than about 0.06, less than about 0.05, less than about 0.04, less than about 0.03, less than about 0.02 or less than about 0.01. In a further example, the ratio of IC₅₀ (PLK2)/IC₅₀ (PLK3) is less than about 0.009, less than about 0.008, less than about 0.007, less than about 0.006, less than about 0.005, less than about 0.004, less than about 0.003, less than about 0.002 or less than about 0.001. In yet another example, the ratio of IC₅₀ (PLK2)/IC₅₀ (PLK3) is less than about 0.0009, less than about 0.0008, less than about 0.0007, less than about 0.0006, less than about 0.0005, less than about 0.0004, less than about 0.0003, less than about 0.0002 or less than about 0.0001.

Certain compounds as described herein are characterized by the following inhibitory activities involving PLK2, PLK1 and PLK3. In one example, the ratio of IC₅₀ (PLK2)/IC₅₀ (PLK1) is less than about 1, less than about 0.9, less than about 0.8, less than about 0.7, less than about 0.6, less than about 0.5, less than about 0.4, less than about 0.3, less than about 0.2 or less than about 0.1 and the ratio of IC₅₀ (PLK2)/IC₅₀ (PLK3) is each than about 1, less than about 0.9, less than about 0.8, less than about 0.7, less than about 0.6, less than about 0.5, less than about 0.4, less than about 0.3, less than about 0.2 or less than about 0.1. In another example, the ratio of IC₅₀ (PLK2)/IC₅₀ (PLK1) is less than about 0.09, less than about 0.08, less than about 0.07, less than about 0.06, less than about 0.05, less than about 0.04, less than about 0.03, less than about 0.02 or less than about 0.01 and the ratio of IC₅₀ (PLK2)/IC₅₀ (PLK3) is less than about 0.09, less than about 0.08, less than about 0.07, less than about 0.06, less than about 0.05, less than about 0.04, less than about 0.03, less than about 0.02 or less than about 0.01. In a further example, the ratio of IC₅₀ (PLK2)/IC₅₀ (PLK1) is less than about 0.009, less than about 0.008, less than about 0.007, less than about 0.006, less than about 0.005, less than about 0.004, less than about 0.003, less than about 0.002 or less than about 0.001 and the ratio of IC₅₀ (PLK2)/IC₅₀ (PLK3) is less than about 0.009, less than about 0.008, less than about 0.007, less than about 0.006, less than about 0.005, less than about 0.004, less than about 0.003, less than about 0.002 or less than about 0.001. In yet another example, the ratio of IC₅₀ (PLK2)/IC₅₀ (PLK1) is less than about 0.0009, less than about 0.0008, less than about 0.0007, less than about 0.0006, less than about 0.0005, less than about 0.0004, less than about 0.0003, less than about 0.0002 or less than about 0.0001 and the ratio of IC₅₀ (PLK2)/IC₅₀ (PLK3) is less than about 0.0009, less than about 0.0008, less than about 0.0007, less than about 0.0006, less than about 0.0005, less than about 0.0004, less than about 0.0003, less than about 0.0002 or less than about 0.0001.

Certain compounds as described herein are characterized by the following inhibitory activities involving PLK2 and other kinases. In one example, the ratio of IC₅₀ (PLK2)/IC₅₀ (Kinase) is less than about 1, less than about 0.9, less than about 0.8, less than about 0.7, less than about 0.6, less than about 0.5, less than about 0.4, less than about 0.3, less than about 0.2 or less than about 0.1. In another example, the ratio of IC₅₀ (PLK2)/IC₅₀ (Kinase) is less than about 0.09, less than about 0.08, less than about 0.07, less than about 0.06, less than about 0.05, less than about 0.04, less than about 0.03, less than about 0.02 or less than about 0.01. In a further example, the ratio of IC₅₀ (PLK2)/IC₅₀ (Kinase) is less than about 0.009, less than about 0.008, less than about 0.007, less than about 0.006, less than about 0.005, less than about 0.004, less than about 0.003, less than about 0.002 or less than about 0.001. In yet another example, the ratio of IC₅₀ (PLK2)/IC₅₀ (Kinase) is less than about 0.0009, less than about 0.0008, less than about 0.0007, less than about 0.0006, less than about 0.0005, less than about 0.0004, less than about 0.0003, less than about 0.0002 or less than about 0.0001. Where preferably (Kinase) is one or more kinases selected from the group consisting of CDK-1, CDK-2, CDK-5, CLK-1, CLK-2, CLK-3, CLK-4, NEK-1, NEK-2, NEK-4, NEK-6, NEK-7, MAP4K4 and STK16.

Certain compounds as described herein are characterized by the following inhibitory activities involving PLK2 and other kinases. In one example, the ratio of [% inhibition at 10 μM (Kinase)]/[% inhibition at 10 μM (PLK2)] is less than about 1, less than about 0.9, less than about 0.8, less than about 0.7, less than about 0.6, less than about 0.5, less than about 0.4, less than about 0.3, less than about 0.2 or less than about 0.1. In another example, the ratio of [% inhibition at 10 μM (Kinase)]/[% inhibition at 10 μM (PLK2)] is less than about 0.09, less than about 0.08, less than about 0.07, less than about 0.06, less than about 0.05, less than about 0.04, less than about 0.03, or less than about 0.02. Where preferably (Kinase) is one or more kinases selected from the group consisting of CDK-1, CDK-2, CDK-5, CLK-1, CLK-2, CLK-3, CLK-4, NEK-1, NEK-2, NEK-4, NEK-6, NEK-7, MAP4K4 and STK16.

### In Vivo Activities

Certain compounds as described herein exhibit in vivo biological activities, such as the reduction of alpha-synuclein phosphorylation in the brain of a test animal. An in vivo model, which can be used to assess the potential in vivo beneficial effect of the compounds as described herein, is described in Example B. For example, mice dosed with the compounds as described herein show reduced levels of phosphorylated alpha-synuclein (e.g., p-Ser-129-alpha-synuclein) in their brain tissue (e.g., cerebral cortex) when compared to mice treated with vehicle.

Certain compounds as described herein are characterized by the following in vivo biological activities involving the concentration of p-Ser-129-alpha-synuclein and total alpha-synuclein in the brain tissue (e.g., cerebral cortex) of a test animal (e.g., rodent, such as mice, rat, rabbit and the like). In one example, administration of a compound as described herein to a test animal (e.g., at a dose of about 50 mg, about 100 mg, about 200 mg or about 300 mg/kg), results in a reduction of the p-Ser-129-alpha-synuclein/total alpha-synuclein ratio in the brain tissue of the test animal by at least about 1%, at least about 2%, at least about 3%, at least about 4%, at least about 5%, at least about 6%, at least about 7%, at least about 8%, at least about 9% or at least about 10% relative to the p-Ser-129-alpha-synuclein/total alpha-synuclein ratio found in the brain tissue of a comparable, untreated (vehicle treated) test animal. In another example, administration of a compound as described herein to a test animal (e.g., at a dose of about 50 mg, about 100 mg, about 200 mg or about 300 mg/kg), results in a reduction of the p-Ser-129-alpha-synuclein/total alpha-synuclein ratio in the brain tissue of the test animal by at least about 11%, at least about 12%, at least about 13%, at least about 14%, at least about 15%, at least about 16%, at least about 17%, at least about 18%, at least about 19% or at least about 20% relative to the p-Ser-129-alpha-synuclein/total alpha-synuclein ratio found in brain tissue of a comparable, untreated (vehicle treated) test animal.

In yet another example, administration of a compound as described herein to a test animal (e.g., at a dose of about 50 mg, about 100 mg, about 200 mg or about 300 mg/kg), results in a reduction of the p-Ser-129-alpha-synuclein/total alpha-synuclein ratio in the brain tissue of the test animal by at least about 21%, at least about 22%, at least about 23%, at least about 24%, at least about 25%, at least about 26%, at least about 27%, at least about 28%, at least about 29% or at least about 30% relative to the p-Ser-129-alpha-synuclein/total alpha-synuclein ratio found in brain tissue of a comparable, untreated (vehicle treated) test animal. In a further example, administration of a compound as described herein to a test animal (e.g., at a dose of about 50 mg, about 100 mg, about 200 mg or about 300 mg/kg), results in a reduction of the p-Ser-129-alpha-synuclein/total alpha-synuclein ratio in the brain tissue of the test animal by at least about 31%, at least about 32%, at least about 33%, at least about 34%, at least about 35%, at least about 36%, at least about 37%, at least about 38%, at least about 39% or at least about 40% relative to the p-Ser-129-alpha-synuclein/total alpha-synuclein ratio found in brain tissue of a comparable, untreated (vehicle treated) test animal. In yet another example, administration of a compound as described herein to a test animal (e.g., at a dose of about 50 mg, about 100 mg, about 200 mg or about 300 mg/kg), results in a reduction of the p-Ser-129-alpha-synuclein/total alpha-synuclein ratio in the brain tissue of the test animal by at least about 41%, at least about 42%, at least about 43%, at least about 44%, at least about 45%, at least about 46%, at least about 47%, at least about 48%, at least about 49% or at least about 50% relative to the p-Ser-129-alpha-synuclein/total alpha-synuclein ratio found in brain tissue of a comparable, untreated (vehicle treated) test animal. In yet another example, administration of a compound as described herein to a test animal (e.g., at a dose of about 50 mg, about 100 mg, about 200 mg or about 300 mg/kg), results in a reduction of the p-Ser-129-alpha-synuclein/total alpha-synuclein ratio in the brain tissue of the test animal by at least about 51%, at least about 52%, at least about 53%, at least about 54%, at least about 55%, at least about 56%, at least about 57%, at least about 58%, at least about 59% or at least about 60% relative to the p-Ser-129-alpha-synuclein/total alpha-synuclein ratio found in brain tissue of a comparable, untreated (vehicle treated) test animal.

### Synthesis of the Compounds of the Invention

The compounds as described herein can be prepared using methods known in the art of organic synthesis and those described herein in the Examples. The starting materials and various intermediates may be obtained from commercial sources, prepared from commercially available compounds, and/or prepared using known synthetic methods. For example, the compounds as described herein, as well as all intermediates, can be synthesized by known processes using either solution or solid phase techniques. Exemplary procedures for preparing compounds as described herein are outlined in the following schemes.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P.G. M. Wuts, Protecting Groups in Organic Synthesis, Third Edition, Wiley, New York, 1999, and references cited therein.

In one example, compounds as described herein, e.g. compounds of Formula (I), can be prepared from, for example, the chloro substituted compound E or E¹, which are prepared using a procedure outlined in Scheme 1, below:

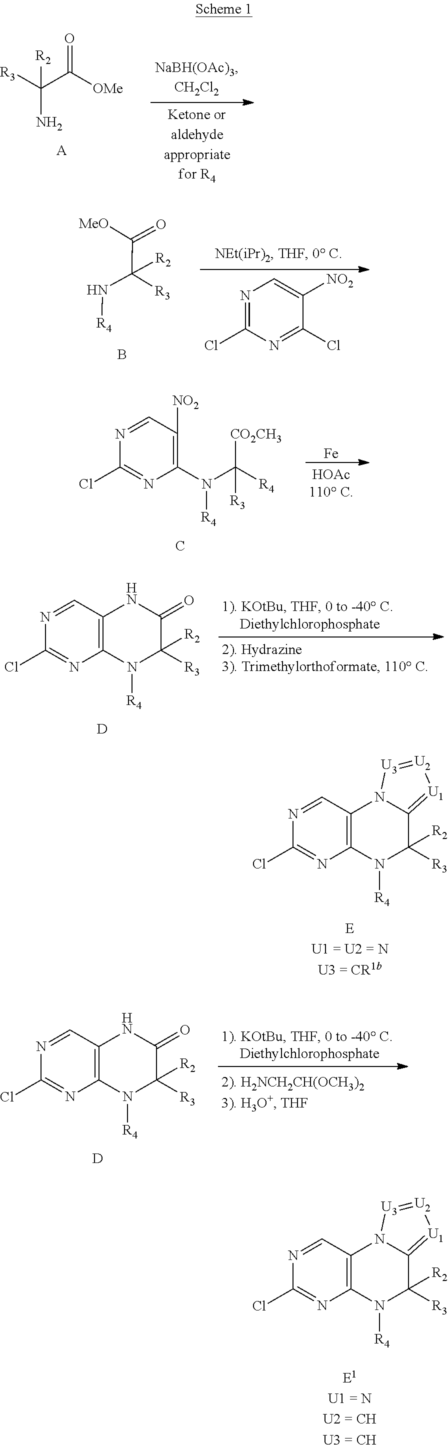

In Scheme 1, U¹, U², U³, R², R³ and R⁴ are as defined herein (see, e.g., Formula (I)). Compound B can be prepared from Compound A by the reductive amination of amino acid ester followed by coupling with 2,4-dichloro-5-nitropyrimidine to form Compound C, which can be accomplished by a variety of synthetic methods. To prepare N-substituted amino acid esters, such as Compound B, from the unsubstituted amino acid Compound A and an aldehyde or ketone appropriate for R₄, sodium triacetoxy borohydride is especially suitable for reductive animations (A. F. Abdel-Magid, K. G. Carson, B. D. Harris, C. A. Maryanoff, R. D. Shah, J. Org. Chem., 1996, 61, 3849-3862) under a range of temperatures (˜78° C. to reflux) in alcoholic or chlorocarbon or other aprotic non-polar solvents with or without catalytic acetic acid. An alternative reagent for reductive amination is sodium cyanoborohydride (Ellen W. Baxter, Allen B. Reitz, Reductive Aminations of Carbonyl Compounds with Borohydride and Borane Reducing Agents in Organic Reactions, 2002, John Wiley and Sons). This reagent can be used effectively in alcoholic or non-polar aprotic solvents at a range of temperatures (˜78° C. to reflux) often with a catalytic amount of acetic acid added to enhance the generation of the required imine intermediate in situ. N-arylation of 2,4-dichloro-5-nitropyrimidine (Compound B to Compound C) can be accomplished by a variety of methods. The Buchwald-Hartwig amination is a general method that could lead to useful amounts of compound C (John P. Wolfe and Stephen L. Buchwald (2004), (Palladium-Catalyzed Amination Of Aryl Halides And Aryl Triflates, Org. Synth., Coll. Vol. 10: 423; Frederic Paul, Joe Patt, John F. Hartwig (1994) Palladium-catalyzed formation of carbon-nitrogen bonds. Reaction intermediates and catalyst improvements in the hetero cross-coupling of aryl halides and tin amides J. Am. Chem. Soc. 116: 5969-5970). However, the 5-nitro group of this pyrimidine analog activates the 4-Cl towards displacement and often leads to preferential N-arylation at the 4-position over the 2-position using simple base-promoted nucelophilic substitution chemistry. Typical bases used can be alkoxide, NaH, NaOH, K₂CO₃, Na₂CO₃ or trialkylamines; temperature may range from −78° C. to reflux temperature of the solvent; solvents used may be polar or non-polar aprotic solvents included DMF, acetonitrile, chlorocarbon solvents, THF or DME.

Ring closure of compound C is conveniently effected by iron reduction of the nitro functionality in hot acetic acid to give the cyclized Pteridine ring Compound D. The temperatures used for this conversion can range from ambient temperature to the reflux point of the solvent. Alternative approaches to the transformation of Compound C to Compound D can use a stepwise reduction of the nitro group to an amine using a variety of reagents (Pd—C/H₂; SnCl₄; Ph₃P/H₃O⁺, etc) followed by cyclization to D in a wide variety of solvents ranging from water, alcohols and glacial acetic acid to non-polar aprotic (chloroform, THF) and polar aprotic solvents (DMF, acetonitrile) at temperatures ranging from ambient to reflux temperature of the solvent. Furthermore, in some cases it can be advantageous to catalyze the ring closure by the addition of a catalytic amount of acid such as a few drops of mineral acid, acetic acid of an arylsulfonic acid.

Triazole analogs where U¹=U²=—N— and U³=—CR^{1b}— (Compound E) can be prepared from Compound D by a three step procedure involving the generation of an iminum phosphonate intermediate using strong base (such as KOtBu, LDA, LiHMDS, NaH) to first deprotonate the amine —NH—, the resulting oxyanion is then quenched with diethylchlorophosphate to form the iminium phosphonate. Solvents most advantageous for these transformations are ethers, such as diethylether, THF, dioxane and DME; temperatures used for this step range from ambient to −78° C. At ambient temperature excess hydrazine is added and the mixture is stirred at this temperature from one to 48 hours. The hydrazine addition product may then be isolated and purified by chromatography or carried forward crude; it is taken up in the appropriate trialkyl ortho-acid ester (e.g. trimethyl orthoformate of the example provides R^{1b} as H, orthoacetate provides R^{1b} as methyl) and stirred at temperatures ranging from ambient to the reflux point of the ortho-acid ester for periods of 0.25 to 8 hours.

Imidazole analogs in which U¹=—N— and U²=U³=—CH— (Compound E¹) can be prepared from Compound D from the same iminium phosphate intermediate by quenching with (1,1-dimethoxy-2-amino)ethanol in non-polar aprotic solvents such as THF, DME, ethyl ether or dioxane at temperatures ranging from 0° C. to reflux followed by hydrolysis with aqueous mineral acid and cyclization in the same media with or without heating.

The final steps used in the formation of such triazole or imidazole analogs (i.e. conversion of Compound D to Compounds E or E′) can be applied to a variety of compounds having the pteridinone core of Compound D, such as those compounds as described in PCT International Publication Number WO 2011/079118.

In one example, compounds of Formula (I) are prepared from E or E¹ as outlined in Scheme 2, below:

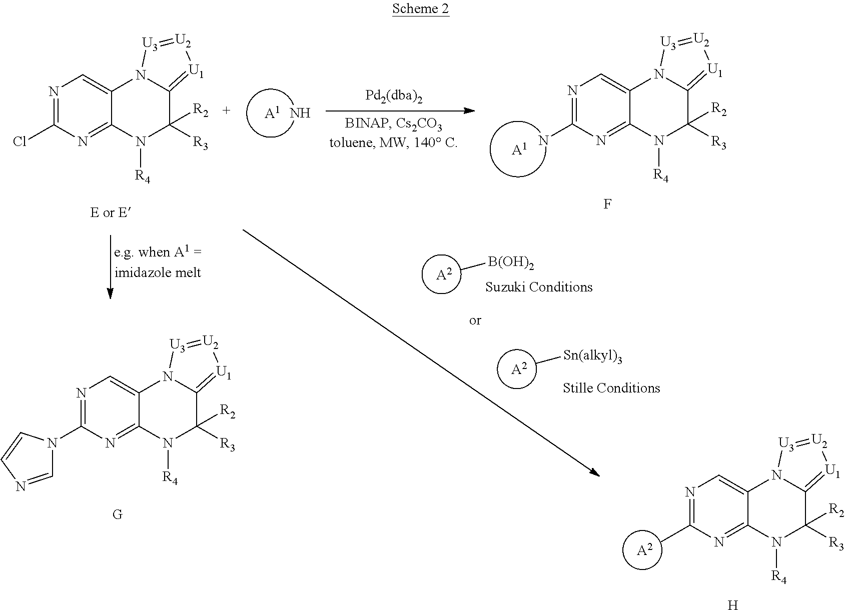

Compound F where the group A¹ is attached to the Pteridine core via a N-atom embedded within the cyclic heteroalkyl or cyclic heteroaryl can be derived from compound E or E¹ by the Buchwald-Hartwig coupling [John P. Wolfe and Stephen L. Buchwald (2004), (Palladium-Catalyzed Amination Of Aryl Halides And Aryl Triflates, Org. Synth., Coll. Vol. 10: 423; Frederic Paul, Joe Patt, John F. Hartwig (1994) Palladium-catalyzed formation of carbon-nitrogen bonds; Reaction intermediates and catalyst improvements in the hetero cross-coupling of aryl halides and tin amides J. Am. Chem. Soc. 116: 5969-5970] of an amine ranging from a simple cyclic heteroalkylamine (such as morpholine, pyrrolidine, piperidine, piperizine, etc.) to a cyclic heteroarylamine (such as C-substituted or unsubstituted imidazoles and C-substituted or unsubstituted pyrazoles). Alternatively, Product F with simple cyclic heteroalkylamine (such as morpholine, pyrrolidine, piperidine, piperizine, etc.) can be obtained by heating an excess of the liquid amine neat with E or E¹ at 50° C. to 150° C. for 1 to 8 h. Compound G where Ring A¹ is unsubstituted imidazole can be obtained by simply melting a mixture of E or E¹ with excess imidazole. Compound H can be synthesized from E or E¹ using either Suzuki coupling conditions (Suzuki, A. Pure Appl. Chem. 1991, 63, 419-422) with the requisite boronic acids or Stille coupling conditions (Stille, J. K. Angew. Chem. Int. Ed. Engl. 1986, 25, 508-524) with the requisite trialkyltin analog.

In one example, compounds as described herein, e.g. compounds of Formula (I), may be prepared using the methods of Schemes 1 and 2, for example Compound D can be reacted similarly to Scheme 2 to form Compounds D′ and D″, which are then reacted following the protocols of Scheme 1, resulting in F or H directly from D′ or D″, respectively, as outlined in Scheme 3 below:

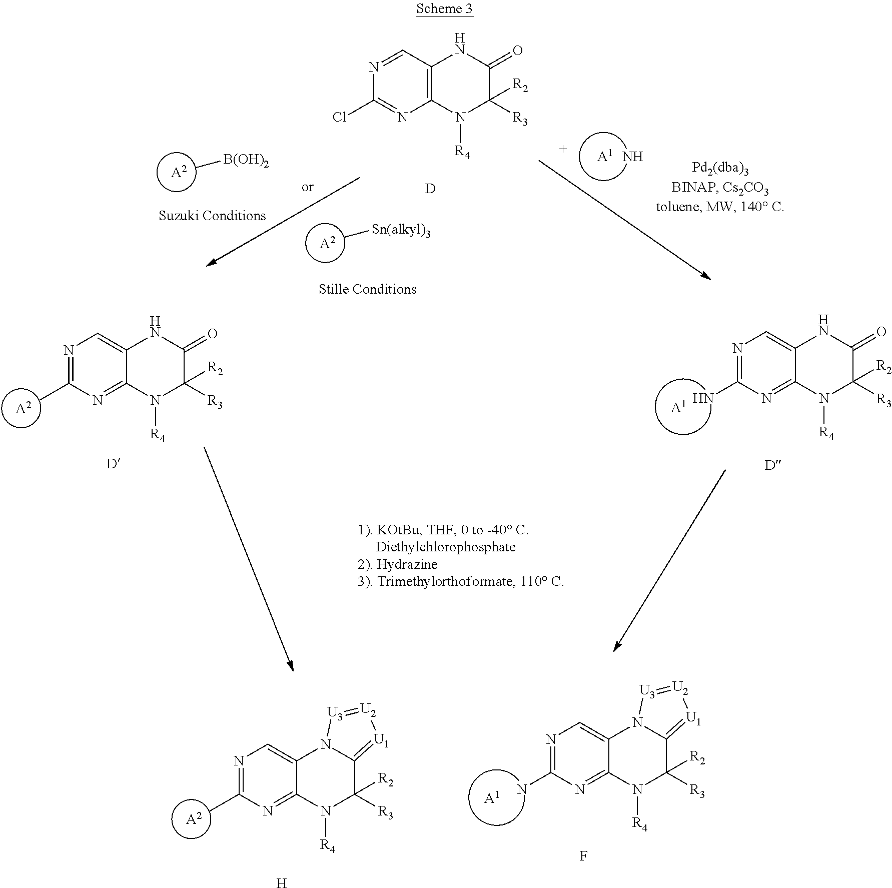

In one example, compounds as described herein, e.g. compounds of Formula (I), may be prepared using the methods of Schemes 1 and 2, for example Compound C can be reacted similarly to Scheme 2 to form Compounds C′ and C″ prior to reacting to form D (to give D′ or D″, respectively). D′ or D″ is then reacted following the protocols of Scheme 1, resulting in F or H from D′ or D″, respectively, as outlined in Scheme 4 below:

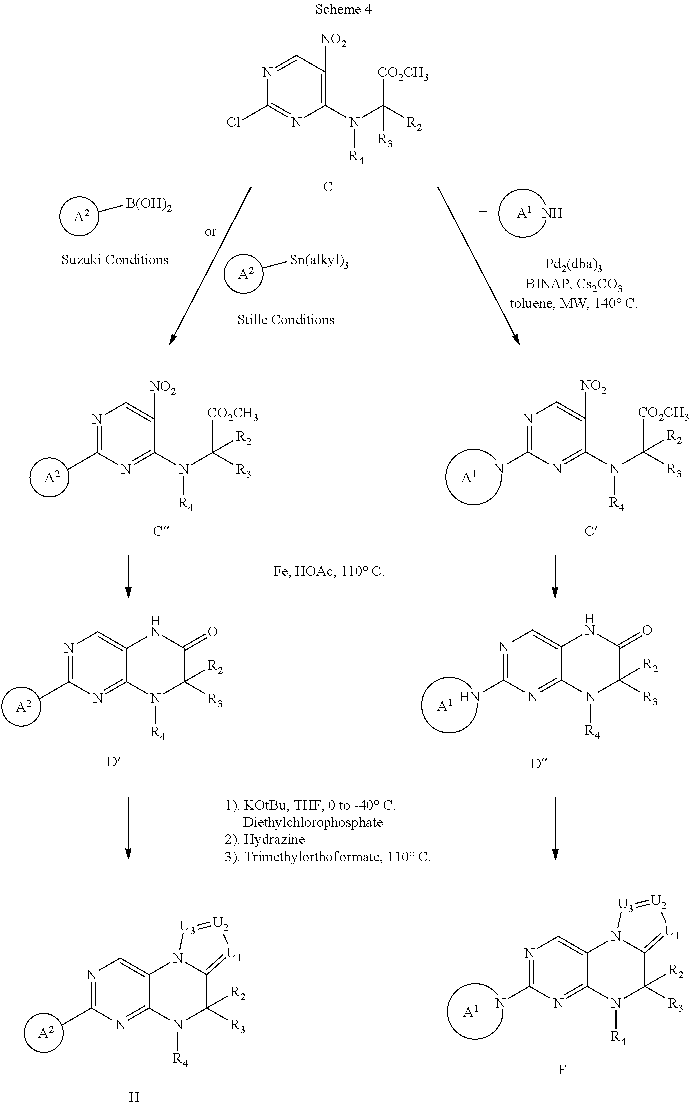

### Boronic Acid Reagents

In Schemes 3 and 4, the boronic acid reagent can be any aryl- or heteroaryl boronic acid or ester thereof. Exemplary boronic acid reagents include:

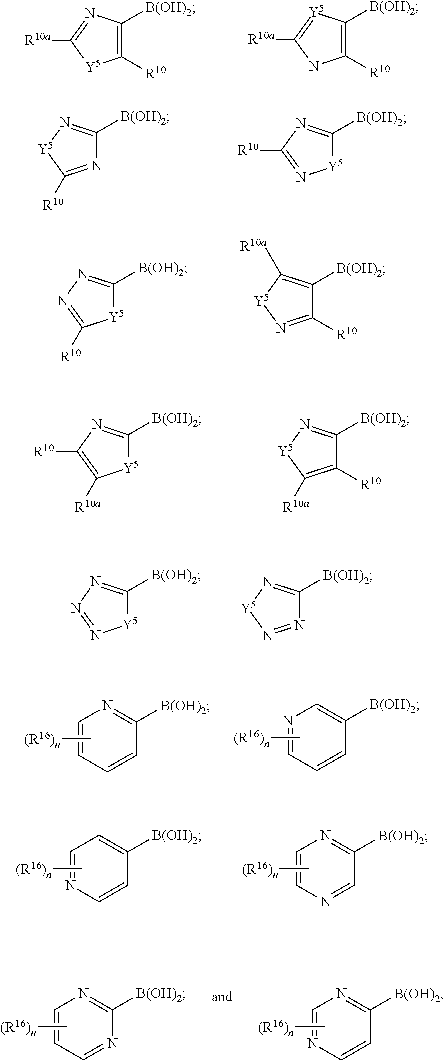

wherein n is an integer selected from 0 to 4 and m is an integer selected from 0 to 3. Y⁵ is a member selected from O, S and NR¹¹, wherein R¹¹ is defined herein (e.g., R¹¹ is a member selected from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl).

In the boronic acid reagents above, R¹⁰, R^{10a} and each R¹⁶ are defined as herein above. In one example, R¹⁰, R^{10a} and each R¹⁶ are members independently selected from H, substituted or unsubstituted C₁-C₁₀-alkyl, substituted or unsubstituted 2- to 10-membered heteroalkyl, substituted or unsubstituted C₃-C₈-cycloalkyl, substituted or unsubstituted 3- to 8-membered heterocycloalkyl, substituted or unsubstituted aryl, substituted or unsubstituted heteroaryl, CN and halogen. Two members selected from R¹⁰, R^{10a} and R¹¹, when on adjacent ring atoms, together with the atoms to which they are attached, are optionally joined to form a 5- to 7-membered ring.

### Catalyst

In Schemes 3 and 4, the catalyst can be any catalyst useful to affect C—C cross coupling reactions, such as Suzuki-type reactions. Such catalysts are known to those of skill in the art and include transition metal catalysts, such as palladium catalysts. Exemplary catalysts include Pd(OAc)₂ in combination with a ligand, as well as preformed Pd complexes, such as Pd(dppf)Cl₂ and the like.

### Pharmaceutical Compositions

The invention further provides pharmaceutical compositions including a compound as described herein, e.g., those of Formulae (I) to (XVII) (or any embodiment thereof), and at least one pharmaceutically acceptable carrier. The term “pharmaceutically acceptable carrier” means all pharmaceutically acceptable ingredients known to those of skill in the art, which are typically considered non-active ingredients. The term “pharmaceutically acceptable carrier” includes solvents, solid or liquid diluents, vehicles, adjuvants, excipients, glidants, binders, granulating agents, dispersing agents, suspending agents, wetting agents, lubricating agents, disintegrants, solubilizers, stabilizers, emulsifiers, fillers, preservatives (e.g., anti-oxidants), flavoring agents, sweetening agents, thickening agents, buffering agents, coloring agents and the like, as well as any mixtures thereof. Exemplary carriers (i.e., excipients) are described in, e.g., Handbook of Pharmaceutical Manufacturing Formulations, Volumes 1-6, Niazi, Sarfaraz K., Taylor & Francis Group 2005, which is incorporated herein by reference in its entirety. A pharmaceutical composition of the invention may include one or more compounds of the invention in association with one or more pharmaceutically acceptable carrier and optionally other active ingredients.

The compounds of the invention may be administered orally, topically, parenterally, by inhalation or spray or rectally in dosage unit formulations containing at least one pharmaceutically acceptable carrier. The term “parenteral” as used herein includes percutaneous, subcutaneous, intravascular (e.g., intravenous), intramuscular, or intrathecal injection or infusion techniques and the like. The pharmaceutical compositions containing compounds of the invention may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The compounds of the invention may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of the invention may be administered parenterally in a sterile medium. The compound, depending on the vehicle and concentration used, can either be suspended or dissolved in the vehicle. Advantageously, adjuvants such as local anesthetics, preservatives and buffering agents can be dissolved in the vehicle.

For disorders of the eye or other external tissues, e.g., mouth and skin, the formulations are preferably applied as a topical gel, spray, ointment or cream, or as a scleral suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound, which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or oil or with both a fat and an oil. Preferably, a hydrophilic emulsifier is included together with a lipophilic emulsifier, which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base, which forms the oily, dispersed phase of the cream formulations. Emulsifiers and emulsion stabilizers suitable for use in the formulation of the invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The anti-inflammatory active ingredients are preferably present in such formulations in a concentration of 0.5 to 20%, advantageously 0.5 to 10% and particularly about 1.5% w/w. For therapeutic purposes, the active compounds of this combination invention are ordinarily combined with one or more adjuvants appropriate to the indicated route of administration. The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.005 mg to about 100 mg per kilogram of body weight per day are useful in the treatment of the diseases and conditions described herein (e.g., about 0.35 mg to about 7 g per human patient per day, based on an average adult person weight of 70 kg). The amount of active ingredient that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may be preferable to apply a topical preparation of compounds of this invention to the affected area one to four times a day.

Formulations suitable for inhalation or insufflation include solutions and suspensions in pharmaceutically acceptable aqueous or organic solvents, or mixtures thereof, and powders. The liquid or solid compositions may contain suitable pharmaceutically acceptable excipients as describe above. The compositions may be administered by oral or nasal respiratory route for local or systemic effect. Compositions may be nebulized by use of inert gases or vaporized, and breathed directly from the nebulizing/vaporizing device or the nebulizing device may be attached to a facemask tent or intermittent positive pressure-breathing machine.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water.

### Methods

Over-activation of PLK2 is believed to be an important mechanism in the formation of Lewy bodies and is thus implicated in diseases, which are characterized by the formation of Lewy bodies. Over-activation of PLK1 is implicated in a variety of cancers. Certain compounds of the invention exhibit inhibitory activity against PLKs (e.g., PLK1, PLK2 and PLK3). Kinase activity can be determined using a kinase assay, which typically employs a kinase substrate and a phosphate group donor, such as ATP (or a derivative thereof). Exemplary kinase substrates for various kinases are described in Example A. The kinase catalyzes the transfer of a phosphate group from the phosphate group donor (e.g., ATP) onto the substrate forming a covalent bond. Compounds of the invention can inhibit the activity of the kinase, slowing the above described reaction and resulting in a smaller number of phosphate groups being transferred. Hence, the current invention provides a method (i.e., an in vitro assay) that includes: (i) contacting a compound of the invention with a kinase (e.g., PLK1, PLK2, PLK3 or other PLK isoform) thereby forming a mixture. The method may further include (ii) contacting the mixture with a kinase substrate (e.g., peptide substrate) and ATP (or a derivative thereof), thereby forming an amount of phosphorylated kinase substrate. The method can further include (iii) measuring the amount of phosphorylated kinase substrate. The amount of phosphorylated substrate may be measured using a detection reagent. Suitable detection reagents can include a metal reagent, such as a lanthanoid (e.g., Eu-63), a radioactive probe, a labeled (e.g., fluorescently labelled) antibody and combinations thereof. In one example, the assay is a fluorescence resonance energy transfer (FRET) assay (e.g., TR-FRET). Examples of such assays are described in Example A. In a particular embodiment, a compound of the invention is used as a reference standard to determine the in vitro activity of other compounds in a kinase assay as described above. Thus, in another example, the compound of the invention is used in an in vitro assay for identifying candidate compounds that are capable of inhibiting PLK (e.g., PLK1, PLK2 and PLK3). In one example, in the above described methods, the kinase is PLK2.

### Methods of Treatment

Compounds and compositions of the invention are useful in the treatment and/or prevention of PLK mediated disorders, including PLK1 mediated diseases such as cancers and PLK2 mediated diseases such as neurodegenerative diseases (e.g., Lewy body diseases) described herein. An in vivo model, which can be used to assess the potential in vivo beneficial effect of the compounds of the invention, is described in Example B.

In one example, the invention provides a method of treating a disease. The method includes administering to a mammalian subject (e.g., human) in need thereof a therapeutically effective amount of a compound or salt of the invention, for example those according to any one of Formulae (I) to (XVII) (or any embodiment thereof), or a composition comprising such compounds or salts. Exemplary diseases, which can be treated with the compounds and compositions of the invention include neurodegenerative diseases, and especially alpha-synucleinopathies, e.g, those associated with the formation of Lewy bodies (Lewy body diseases or those associated with the formation of glial cortical inclusions). Lewy body diseases (LBDs) are characterized by the formation of Lewy bodies (LBs) and may further be associated with degeneration of the dopaminergic system, motor alterations and cognitive impairment and include Parkinson's disease and dementia with Lewy bodies (DLB), which is a type of dementia closely allied to Parkinson's disease. It is characterized anatomically by the presence of Lewy bodies—clumps of alpha-synuclein and ubiquitin protein in neurons (e.g., detectable in post-mortem brain biopsies). Multiple system atrophy (MSA) is an exemplary disease associated with the formation of glial cortical inclusions.

Thus, compounds as described herein that are PLK2 inhibitors can be used to treat alpha-synucleinopathies, which include without limitation Lewy body diseases such as Parkinson's disease (PD), Parkinson disease with dementia (PDD), PD at risk syndrome (PARS), dementia with Lewy bodies (DLB) (i.e., diffuse Lewy body disease (DLBD), Lewy body dementia, Lewy body disease, cortical Lewy body disease or senile dementia of Lewy type), Lewy body variant of Alzheimer's disease (LBV) (i.e., diffuse Lewy body type of Alzheimer's disease), combined Parkinson's disease (PD) and Alzheimer's disease (AD), as well as diseases associated with glial cortical inclusions, such as syndromes identified as multiple system atrophy (MSA), including striatonigral degeneration, olivopontocerebellar atrophy, and Shy-Drager syndrome.

Compounds as described herein that are PLK2 inhibitors can also be used to treat disease with Parkinson-like symptoms, such as Hallervorden-Spatz syndrome (also referred to as Hallervorden-Spatz disease), fronto-temporal dementia, Sandhoff disease, progressive supranuclear palsy (PSP), and corticobasal degeneration (CBD).

In a particular example, the neurodegenerative disease is Parkinson's disease, dementia with Lewy bodies (DLB), diffuse Lewy body type of Alzheimer's disease or multiple system atrophy (MSA). Thus, in one example, the invention provides a method of treating Parkinson's disease, dementia with Lewy bodies (DLB), diffuse Lewy body type of Alzheimer's disease or multiple system atrophy (MSA), comprising administering to a mammalian subject (e.g., human) in need of such treatment, a therapeutically effective amount of a compound or composition of any one of Formula (I) to (XVI) (or any embodiment thereof).

Other diseases, which can be treated with the compounds and compositions of the invention also include any conditions associated with the disease, e.g., Parkinsonism, autonomic dysfunctions (e.g., Shy-Drager syndrome, postural or orthostatic hypotension), cerebellar dysfunctions, ataxia, movement disorders, cognitive deterioration, sleep disorders, hearing disorders, tremors, rigidity (e.g., joint stiffness, increased muscle tone), bradykinesia, akinesia and postural instability (failure of postural reflexes, along other disease related factors such as orthostatic hypotension or cognitive and sensory changes, which lead to impaired balance and falls).

Other neurodegenerative diseases which may be treated by the compounds of this invention include, but are not limited to Alzheimer's disease, Down syndrome, dementia, mild cognitive impairment (MCI), amyotrophic lateral sclerosis (ALS) (Lou Gehrig's Disease), traumatic brain injuries, cerebral ischemic brain damage, ischemic or hemorrhaging stroke, hereditary cerebral hemorrhage with amyloidosis of the dutch-type and cerebral amyloid angiopathy. Neurodegenerative diseases also includes epilepsy, seizures, traumatic brain injury, neurodegenerative disease caused by traumatic injury, ischemia/reperfusion in stroke, ischemic and hemorrhaging stroke, cerebral ischemias, acute hypoxia and ischemia or glutamate neurotoxicity.

The association of cancers with polo-like kinases is well known. It has been established that PLK1 over expression inhibits the function of the tumor suppressor p53 (Ando, Kiyohiro, et al., Nichidai Igaku Zasshi (2003), 62(9), 496-501). The presence of PLK1 correlates with the severity of disease and survival in patients with glioma (Duan et al., Xiandai Zhongliu Yixue (2007), 15(7), 912-913). PKL1 gene plays an important regulatory role in the proliferation of human glioma cells, and RNA interference of PLK1 gene inhibits cell proliferation possibly by suppressing the telomerase activity (Fan, Yu et al., Zhonghua Shenjingyixue Zazhi (2009), 8(1), 5-9). In hepatocellular carcinoma levels of PLK1 expression in tumors correlated with poor patient survival (Pellegrino et al., Hepatology (Hoboken, N.J., United States) (2010), 51(3), 857-868; He, Zi-Li et al., World Journal of Gastroenterology (2009), 15(33), 4177-4182). PLK1 expression appears to be tumor specific in human pancreatic carcinoma (Zhu, Yi, et al., Yixianbingxue (2007), 7(1), 9-12). PLK1 is a prognostic marker in ovarian carcinomas whose over expression correlates with shortened survival times for patients (Weichert, W. et al., British Journal of Cancer (2004), 90(4), 815-821). PLK1 is overexpressed in primary colorectal cancers (Takahashi, Takao, et al., Cancer Science (2003), 94(2), 148-152). Evidence suggest that PLK1 does not act as a cell cycle regulator but plays a constitutive role in papillary carcinoma in the early phase, and may contribute to the malignant transformation of this carcinoma (Ito, Y eta al., British Journal of Cancer (2004), 90(2), 414-418). PLK expression is a marker of proliferation and its expression closely correlates with estrogen receptor expression in human breast cancer (Wolf, Georg et al., Pathology, Research and Practice (2000), 196(11), 753-759). Patients with head and neck squamous cell cancer with moderate rather than high expression levels of PLK had longer 5-year survival rates (Knecht, Rainald et al., Cancer Research (1999), 59(12), 2794-2797). In non-small cell lung cancer, patients with moderate expression of PLK had significantly longer 5-year survival rates than patients with high levels of expression (Wolf, Georg et al., Oncogene (1997), 14(5), 543-549). Thus compounds as described herein that are PLK1 inhibitors can be used to treat oncological disorders, including solid tumors, liquid tumors, tumor metastasis, and without limitation, angiogenic disordors, ocular neovasculization, and infantile haemangiomas. Proliferative diseases which may be treated or prevented by the compounds of this invention include, but are not limited to, acute myelogenous leukemia, chronic myelogenous leukemia, metastatic melanoma, hepatocellular carcinoma, pancreatic carcinoma, brain cancer, lung cancer (e.g. non small cell lung cancer), breast cancer, bladder cancer, thyroid cancer, endometrial cancer, prostate cancer, gastric cancer, oropharyngeal cancer, esophageal cancer, head and neck cancer, ovarian carcinomas, papillary carcinomas, colorectal cancers, hepatoma, melanoma, lymphomas (e.g. non-Hodgkins lymphoma, Hodgkin's lymphoma), advanced metastatic cancers, advanced solid tumors, Kaposi's sarcoma, multiple myeloma and HTLV-1 mediated tumorigenesis. In one embodiment, the cancer, is glioma, glioblastoma, hepatacellular carcinoma, pancreatic carcinoma, colorectal cancer, papillary carcinoma, ovarian carcinoma, non small cell lung cancer, breast cancer, or squamous cell carcinoma.

In another embodiment, the invention provides a method of treating a disease selected from epilepsy, seizures, Huntington's disease, multiple sclerosis, cancer, age-related macular degeneration, diabetic retinopathy and retinal neurodegeneration related to glaucoma or ocular trauma, the method comprising administering to a mammalian subject (e.g., a human subject) in need thereof a pharmaceutically effective amount of a compound or salt of any one of Formulae (I) to (XVII) (or an embodiment thereof) or a pharmaceutical composition comprising at least one compound of Formulae (I) to (XVII) (or an embodiment thereof). Other diseases, which may be treated using the compounds of the invention include alcoholism, Alexander's disease, Alper's disease, ataxia telangiectasia, Batten disease (also known as Spielmeyer-Vogt-Sjogren-Batten disease), prion diseases, bovine spongiform encephalopathy (BSE), Canavan disease, cerebral palsy, Cockayne syndrome, corticobasal degeneration, Creutzfeldt-Jakob disease, frontotemporal lobar degeneration, Huntington's disease, HIV-associated dementia, Kennedy's disease, Krabbe's disease, Lewy body dementia, neuroborreliosis, Machado-Joseph disease (e.g., spinocerebellar ataxia type 3), multiple system atrophy, multiple sclerosis, narcolepsy, Niemann Pick disease, Pelizaeus-Merzbacher disease, Pick's disease, primary lateral sclerosis, progressive supranuclear palsy, Refsum's disease, Sandhoffs disease, Schilder's disease, subacute combined degeneration of spinal cord secondary to pernicious anaemia, spinocerebellar ataxia (multiple types with varying characteristics), spinal muscular atrophy, Steele-Richardson-Olszewski disease and tabes dorsalis.

Autoimmune diseases which may be treated or prevented by the compounds of this invention include, but are not limited to, glomerulonephritis, rheumatoid arthritis, systemic lupus erythematosus, scleroderma, chronic thyroiditis, Graves′ disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis and graft versus host disease (GVHD). The compounds and compositions of the invention are also useful to treat pathologic immune responses such as that caused by T cell activation and thrombin-induced platelet aggregation.

Additional specific conditions or diseases that can be treated with the compounds or compositions of the invention include, without limitation, myocardial ischemia, ischemia/reperfusion in heart attacks, organ hypoxia, vascular hyperplasia, cardiac and renal reperfusion injury, thrombosis, cardiac hypertrophy, hepatic ischemia, liver disease, congestive heart failure, thrombin induced platelet aggregation, endotoxemia and/or toxic shock syndrome, and conditions associated with prostaglandin endoperoxidase synthase-2.

Other specific conditions or diseases that can be treated with the compounds or compositions of the invention include, without limitation, acute pancreatitis, chronic pancreatitis, asthma, allergies, adult respiratory distress syndrome, chronic obstructive pulmonary disease, glomerulonephritis, rheumatoid arthritis, systemic lupus erythematosis, scleroderma, chronic thyroiditis, Grave's disease, diabetes, thrombocytopenia, atopic dermatitis, chronic active hepatitis, myasthenia gravis, multiple sclerosis, inflammatory bowel disease, ulcerative colitis, Crohn's disease, psoriasis, graft versus host disease (GVHD), inflammatory reaction induced by endotoxin, tuberculosis, atherosclerosis, muscle degeneration, cachexia, psoriatic arthritis, Reiter's syndrome, gout, traumatic arthritis, rubella arthritis, acute synovitis, pancreatic beta-cell disease; diseases characterized by massive neutrophil infiltration, rheumatoid spondylitis, gouty arthritis and other arthritic conditions, cerebral malaria, chronic pulmonary inflammatory disease, silicosis, pulmonary sarcoisosis, bone resorption disease, allograft rejections, fever and myalgias due to infection, cachexia secondary to infection, meloid formation, scar tissue formation, ulcerative colitis, pyresis, influenza, osteoporosis, osteoarthritis and multiple myeloma-related bone disorder.

In addition, PLK inhibitors of the instant invention may be capable of inhibiting the expression of inducible pro-inflammatory proteins. Therefore, other “PLK-mediated conditions” which may be treated by the compounds of this invention include edema, analgesia, fever and pain, such as neuromuscular pain, migrains, cancer pain, dental pain and arthritis pain.

In addition to the compounds of this invention, pharmaceutically acceptable derivatives or prodrugs of the compounds of this invention may also be employed in compositions to treat or prevent the above-identified disorders.

The disclosures in this document of all articles and references, including patents, are incorporated herein by reference in their entirety.

The invention is illustrated further by the following examples, which are not to be construed as limiting the invention in scope or spirit to the specific procedures described in them. Analogous structures and alternative synthetic routes within the scope of the invention will be apparent to those skilled in the art.

### EXAMPLES

Reagents and solvents obtained from commercial suppliers were used without further purification unless otherwise stated. Thin layer chromatography was performed on precoated 0.25 mm silica gel plates (E. Merck, silica gel 60, F₂₅₄). Visualization was achieved using UV illumination or staining with phosphomolybdic acid, ninhydrin or other common staining reagents. Flash chromatography was performed using either a Biotage Flash 40 system and prepacked silica gel columns or hand packed columns (E. Merck silica gel 60, 230-400 mesh). Preparatory HPLC was performed on a Varian Prepstar high performance liquid chromatograph. ¹H and ¹³C NMR spectra were recorded at 300 or 400 MHz and 75 MHz, respectively, on a Varian Gemini or Bruker Avance spectrometer. Chemical shifts are reported in parts per million (ppm) downfield relative to tetramethylsilane (TMS) or to proton resonances resulting from incomplete deuteration of the NMR solvent (δ scale). Mass spectra (LCMS) were recorded on an Agilent series 1100 mass spectrometer connected to an Agilent series 1100 HPLC.

In several instances the synthetic examples give a racemic mixture of stereoisomers, which are readily separated by chiral HPLC. The absolute configuration of such compounds is typically assigned based on which is the more active compound against PLK2, consistent with the configuration of several analogs and their known configuration from x-ray co-crystal structures.

LCMS was performed on an Agilent 1100 Series HPLC with a Series 1100 MSD with electrospray ionization using a Phenomenex Luna C18 4.6 mm i.d.×30 mm length, 3μ particle size column. Compound purity was typically determined by HPLC/MS analysis using a variety of analytical methods. Exemplary HPLC methods that may be used in the examples below are as follows:
Analytical Method A: The initial solvent composition is 20% CH₃CN with 0.1% Trifluoroacetic Acid (TFA) and water with 0.1% TFA which ramped to 70% CH₃CN over 10 min., held at 70% for 2 min., then ramped to 95% over 1 min. and held at 95% for 2 minutes with a flow rate of 1.5 ml/minute.
Analytical Method B: The same parameters as Method A changed so that the initial solvent composition is 50% CH₃CN which ramped to 95% CH₃CN over 10 minutes with a flow rate of 1.5 mL/minute.
Analytical Method C: The same parameters as Method A changed so that the initial solvent composition is 20% CH₃CN which ramped to 50% CH₃CN over 10 minutes with a flow rate of 1.5 mL/minute.
Analytical Method D: The same parameters as Method A changed so that the initial solvent composition is 5% CH₃CN which ramped to 20% CH₃CN over 10 minutes with a flow rate of 1.5 mL/minute.
Analytical Method E: Solvent A-Water (0.05% TFA), Solvent B-Acetonitrile (0.05% TFA) with a gradient of 5% B to 95% B in 1.4 min, flow rate: 2.3 mL/min, column: SunFire C18, 4.6*50 mm, 3.5 um, oven temperature: 50° C.

The examples are intended to be illustrative and are not limiting or restrictive to the scope of the invention. For example, where additional compounds are prepared similarly to synthetic methods of another example, or in the same manner as another example, it is understood that conditions may vary, for example, any of the solvents, reaction times, reagents, temperatures, work up conditions, or other reaction parameters may be varied employing alternate solvents, reagents, reaction times, temperatures, work up conditions, and the like, as are readily available to one skilled in the art. Reagents, solvents, and other terms used in the following examples may be referred to in abbreviated forms as are known to one skilled in the art, for example terms and abbreviations are used according to the following table.

| Term or abbreviation | Definition |
|---|---|
| AcOH or HOAc | Acetic acid |
| AcCl | Acetyl chloride |
| BINAP | 2,2′-bis(diphenylphosphino)-1,1′-binaphthyl |
| BnBr | Benzyl bromide |
| BrNBu₄ | Tetrabutylammonium bromide |
| (Boc)₂O | di-tert-butyl dicarbonate |
| tBuOK | Potassium tert-butoxide |
| tBuOH | tert-butanol |
| tBuONO | tert-butyl nitrite |
| mCPBA | meta-Chloroperoxybenzoic acid |
| DAST | Diethylaminosulfur trifluoride |
| DBU | 1,8-dizazbicyclo[5.4.0]undec-7-ene |
| DCM | Dichloromethane (CH₂Cl₂) |
| DCE | 1,2-dichloroethane |
| DIB | (Diacetoxyiodo)benzene |
| DIPEA or Hunig's | N,N-diisopropylethylamine |
| base or NEt(iPr)₂ | |
| DMF | N,N-dimethylformamide |
| DMF-DMA or | Dimethylformamide dimethylacetal |
| DMF DMA | |
| DMAP | 4-Dimethylaminopyridine |
| DME | Dimethyl ether |
| DMSO | Dimethyl sulfoxide |
| DPPP | 1,3-Bis(diphenylphosphino)propane |
| EDCI | 1-Ethyl-3-(3′-dimethylaminopropyl)carbodiimide |
| | hydrochloride |
| EtOAc or EA | Ethyl acetate |
| Et₂O | Diethyl ether |
| Et₂Zn | Diethyl Zinc |
| Et₃N | Triethylamine |
| HATU | 2-(1H-7-Azabenzotriazol-1-yl)--1,1,3,3- |
| | tetramethyl uronium hexafluorophosphate |
| | Methanaminium |
| HOAt | 7-aza-N-hydroxybenzotriazole |
| HMPA | Hexamethylphosphoramide |
| KHMDS | Potassium hexamethyldisilazane |
| LDA | Lithium diisopropylamine |
| LiBHEt₃ | Lithium triethylborohydride |
| MPLC | ISCO CombiFlash ® medium pressure liquid |
| | chromatography system |
| MeCN | Acetonitrile |
| MeOH | Methanol |
| Me₃PO₄ or (MeO)₃PO | Trimethylphosphate |
| or PO(MeO)₃ | |
| NaBH(OAc)₃ | Sodium triacetoxyborohydride |
| NaOAc | Sodium acetate |
| NH(OMe)MeHCl | N,O-dimethylhydroxylammonium chloride |
| NIS | N-iodosuccinimide |
| NMP | N-methyl-2-pyrrolidone |
| Pd(OAc)₂ | Palladium(II) acetate |
| Pd₂(dba)₃ | Tris(dibenzylideneacetone)dipalladium(0) |
| Pd(dppf)Cl₂ | [1,1′-Bis(diphenylphosphino)ferrocene] |
| | dichloropalladium(II), complex with |
| | dichloromethane |
| Pd(PPh₃)₂Cl₂ | Bis (Triphenylphosphine) Palladium Chloride |
| Pd(PPh₃)₄ | Tetrakis(triphenylphosphine)palladium(0) |
| PE | Petroleum Ether |
| PhMe | Toluene |
| PPA | Polyphosphoric acid |
| iPrOH | isopropanol |
| SnBu₃Cl | Tri-n-butylstannyl chloride |
| TEA | Triethylamine |
| THF | Tetrahydrofuran |
| TFA | Trifluoroacetic acid |
| TFAA | Trifluoroacetic anhydride |
| TMSCl | Trimethylsilyl chloride |
| TMSCN | Trimethylsilyl carbonitrile |

### Synthesis of Intermediates

### (R)-Methyl 2-((2-chloro-5-nitropyrimidin-4-yl)(cyclopentyl)amino)butanoate (Intermediate A)

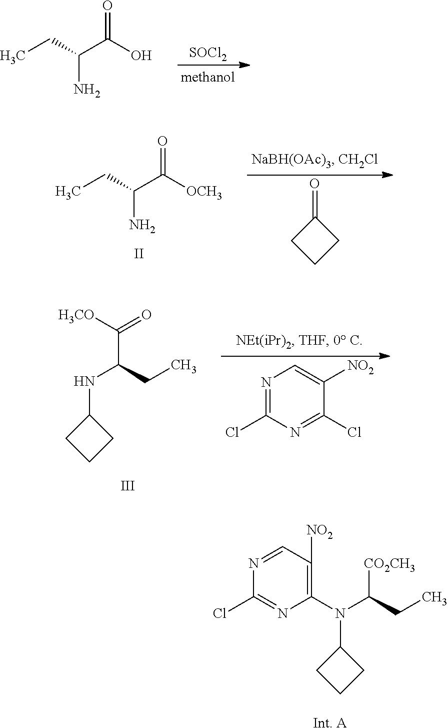

To a suspension of (R)-2-aminobutanoic acid (5.0 g, 48 mmol) in MeOH (27 mL) at −10° C. (ice-salt bath) under N₂ was added dropwise with stirring SOCl₂ (6.4 mL, 86.4 mmol) over 90 min. The flask was fitted with a reflux condenser and heated to 70° C. for 1 hr then cooled to room temperature (rt). The solvent was removed and the residue was dried under high vacuum to afford (R)-methyl 2-aminobutanoate (compound II) as a white powder (7.5 g, 100%).

Compound II (850 mg) and cyclobutanone (540 mg, 1.05 EQ) were dissolved in 8 mL dichloromethane. After the addition of sodium acetate (830 mg, 1.4 EQ) and sodium triacetoxyborohydride (2.3 g, 1.5 EQ) at 0° C., the mixture was stirred for 12 hr at ambient temperature and then 20 mL saturated sodium bicarbonate solution were added. The aqueous phase was extracted with dichloromethane. The combined organic phases were washed with water, dried over MgSO₄ and evaporated down to provide (R)-methyl 2-(cyclobutylamino)butanoate (Compound III), which was taken directly to the next reaction without further purification. LS-MS: [M+H] 172.1.

A mixture of Compound III and Hunig's Base (1.6 mL, 1.2 EQ) in THF (15 mL) was stirred at 0° C. and a solution of 2,4-dichloro-5-nitropyrimidine (1.55 g, 1.1 EQ) in THF (3 mL) at 0° C. was slowly added. After 30 min, the reaction mixture was slowly quenched with brine and diluted with EtOAc (25 mL). The aqueous phase was separated and a normal aqueous workup with EtOAc was followed. The combined organic phases were washed with water, dried over MgSO₄ and evaporated. The residue was purified by silica column (hex: EtOAc=3:1). Yield: 1.1 g (46% 3 steps from Compound I) of (R)-methyl 2-((2-chloro-5-nitropyrimidin-4-yl)(cyclopentyl)amino)butanoate (Intermediate A, yellow solid). LC-MS: [M+H] 329.0.

(R)-methyl 2-((2-chloro-5-nitropyrimidin-4-yl)(cyclopentyl)amino)butanoate (Intermediate E-0), was prepared similarly by the following method:

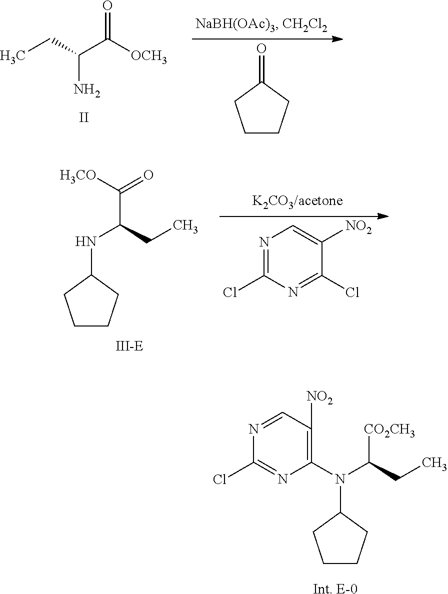

Compound II (7.4 g) and cyclopentanone (4.1 g, 49 mmol) were dissolved in 80 mL DCM. After the addition of sodium acetate (4.0 g, 4 mmol) and sodium triacetoxyborohydride (15.0 g, 71 mmol) at 0° C., the mixture was stirred for 12 hr at rt and then 50 mL saturated sodium bicarbonate solution were added. The aqueous phase was extracted with dichloromethane. The combined organic phases were washed with water, dried over MgSO₄ and evaporated down to give (R)-methyl 2-(cyclopentylamino)butanoate as a light yellow oil (compound III-E, 8.6 g, 95% yield).

Compound III-E (8.6 g) and potassium carbonate (6.0 g, 44 mol) were suspended in 120 mL of acetone. To the mixture was added 2,4-dichloro-5-nitropyrimidine (9.0 g) in 40 mL of acetone at 0° C. After 12 hr, another batch of 2,4-dichloro-5-nitropyrimidine (1.0 g) was added and the mixture was stirred for 4 hr. The reaction mixture was evaporated and the residue partitioned between 800 mL ethyl acetate and 600 mL water. The aqueous phase was extracted with ethyl acetate a second time. The combined organic phases were washed with water, dried over MgSO₄ and evaporated. The residue was purified by silica column (PE: EtOAc=10:1) to give (R)-methyl 2-((2-chloro-5-nitropyrimidin-4-yl)(cyclopentyl)amino)butanoate as a yellow solid (intermediate E-0, 8.0 g, 53% yield).

(R)-Methyl 2-((2-chloro-5-nitropyrimidin-4-yl)(tetrahydro-2H-pyran-4-yl)amino)butanoate (Intermediate M-1), was prepared similarly by the following method:

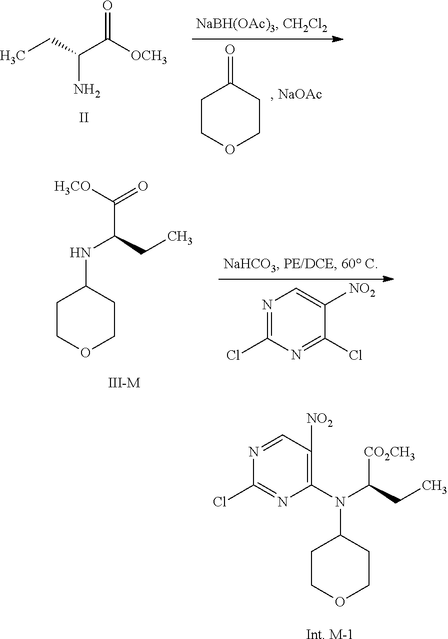

Compound III-M was prepared similarly to the analogous step in the synthesis of Intermediate A, using dihydro-2H-pyran-4(3H)-one instead of cyclobutanone.

To a stirring mixture of compound III-M in petroleum ether:1,2-dichloroethane (2:1, 8 mL total volume), sodium bicarbonate (3.36 g, 4 eq) and 2.4-dichloro-5-nitropyrimidine (2.33 g, 1.2 eq) were added. The resulting mixture was warmed to 60° C. until all the starting material was consumed. This reaction mixture was filtered through a plug of Celite® and the plug was washed several times with dichloromethane.

This mixture was concentrated under reduced pressure and further purified via silica gel chromatography to give Intermediate M-1.

Additional intermediates are prepared similarly to these methods, optionally replacing (R)-2-aminobutanoic acid with a suitable carboxylic acid/ester (in some instances, the product is taken directly to coupling with 2,4-dichloro-5-nitropyrimidine, no reductive amination step) and/or replacing e.g. cyclobutanone with a suitable ketone reactant in the reductive amination step. The following compounds are prepared:
(R)-methyl 2-((2-chloro-5-nitropyrimidin-4-yl)(cyclopentyl)amino)butanoate (Intermediate E-0),
(R)-methyl 2-((2-chloro-5-nitropyrimidin-4-yl)(isopropyl)amino)butanoate (Intermediate G-1),
(R)-methyl 1-(2-chloro-5-nitropyrimidin-4-yl)piperidine-2-carboxylate (Intermediate I-1), and
(R)-methyl 1-(2-chloro-5-nitropyrimidin-4-yl)pyrrolidine-2-carboxylate (Intermediate K-1),
(R)-methyl 2-((2-chloro-5-nitropyrimidin-4-yl)(tetrahydrofuran-3-yl)amino)butanoate (Intermediate N-1),
(R)-methyl 2-((2-chloro-5-nitropyrimidin-4-yl)(cyclopropyl)amino)butanoate (Intermediate 0-1),
methyl 1-((2-chloro-5-nitropyrimidin-4-yl)(isopropyl)amino)cyclopropanecarboxylate (Intermediate R-1),
(R)-methyl 2-((2-chloro-5-nitropyrimidin-4-yl)(3,3,3-trifluoropropyl)amino)butanoate (Intermediate U-1),
(R)-methyl 2-((3-(benzyloxy)cyclobutyl)(2-chloro-5-nitropyrimidin-4-yl)amino)butanoate (Intermediate V-1),
ethyl 1-((2-chloro-5-nitropyrimidin-4-yl)(isopropyl)amino)cyclobutanecarboxylate (Intermediate GG-1),
methyl 2-((2-chloro-5-nitropyrimidin-4-yl)(isopropyl)amino)-2-methylpropanoate (Intermediate HH-1),
methyl 4-(2-chloro-5-nitropyrimidin-4-yl)morpholine-3-carboxylate (Intermediate LL-1),
(R)-methyl 2-((2-chloro-5-nitropyrimidin-4-yl)(oxetan-3-yl)amino)butanoate (Intermediate RR-1),
(2R)-methyl 2-((2-chloro-5-nitropyrimidin-4-yl)(1-cyclopropylethyl)amino)butanoate (Intermediate SS-1),
(R)-methyl 2-((2-chloro-5-nitropyrimidin-4-yl)(perdeuteroisopropyl)amino)butanoate (Intermediate VV-1)
(R)-methyl 2-((2-chloro-5-nitropyrimidin-4-yl)(isopropyl)amino)-2-cyclopropylacetate (Intermediate WW-1),
(R)-tert-butyl 4-((2-chloro-5-nitropyrimidin-4-yl)(1-methoxy-1-oxobutan-2-yl)amino)piperidine-1-carboxylate (Intermediate YY-1), and methyl 2-((2-chloro-5-nitropyrimidin-4-yl)(3,3,3-trifluoropropyl)amino)-2-methylbutanoate (Intermediate ZZ-1).

For Intermediate N-1, the stereochemistry at the 7-position is known to be the R isomer, while the stereochemistry of the tetrahydrofuran ring is not known, but a pure diastereomer is isolated by HPLC. Intermediate N-1 is preferred for use in subsequent reactions, as resulting in more active inhibitors of PLK2. Intermediate N-1 (LCMS: 345.1 m/z (M+H)); ret. Time 5.312 min (Analytical Method A).

The following table provides the Intermediate (column 1), carboxylic acid (or ester, in some instances with no reductive amination) and/or ketone or similar reactant in the reductive amination step (column 2), to give the Intermediate structure as provided in column 3, with LCMS results indicated in column 3.

| Inter- | | |
|---|---|---|
| medi- | Carboxylic acid or red. | |
| ate | animation reactant | Intermediate structure |
| E-0 | 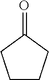 | 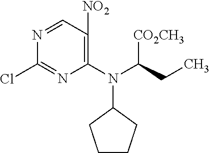 |
| G-1 | 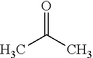 | 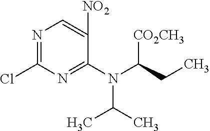 |
| I-1 | 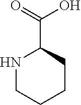 | 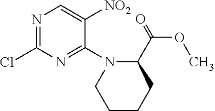 |
| K-1 | 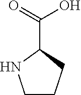 | 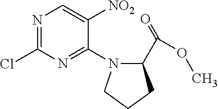 |
| N-1 | 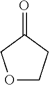 | 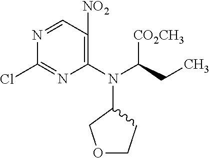 |
| O-1 | 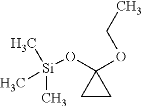 | 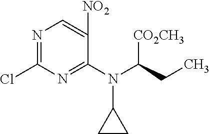 |
| R-1 | 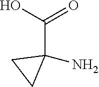 | 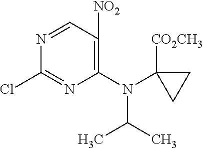 |
| U-1 | 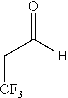 | 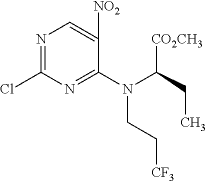 |
| V-1 | 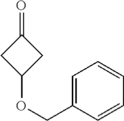 | 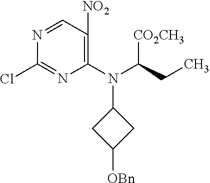 |
| GG-1 | 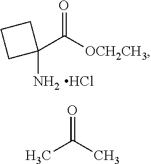 | 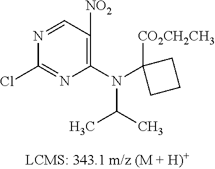 |
| HH-1 | 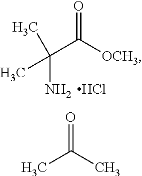 | 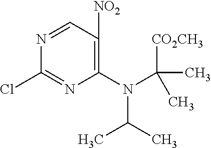 |
| LL-1 | 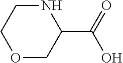 | 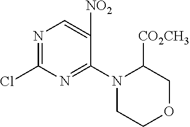 |
| RR-1 | 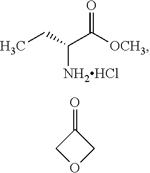 | 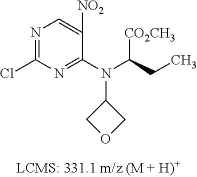 |
| SS-1 | 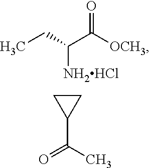 | 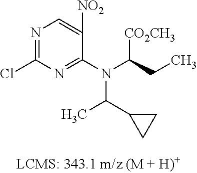 |
| VV-1 | 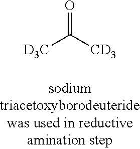 | 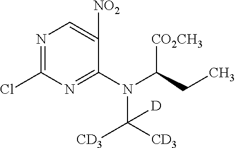 |
| WW-1 | 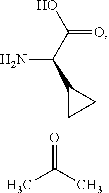 | 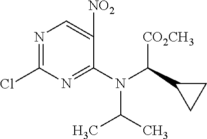 |
| YY-1 | 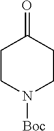 | 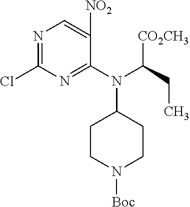 |
| ZZ-1 | 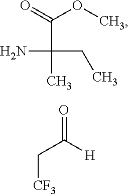 | 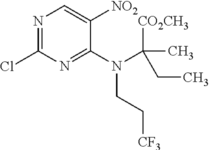 |

### Ethyl 2-((2-chloro-5-nitropyrimidin-4-yl)(isopropyl)amino)-4,4,4-trifluorobutanoate (Intermediate P-1)

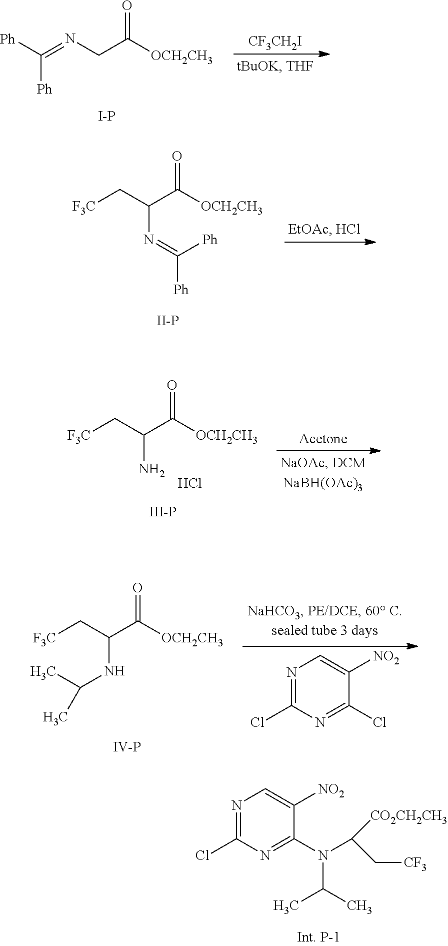

t-BuOK (11.02 g, mmol) was added to 125 mL of DMF and the mixture was stirred at 0° C. for 10 min. Ethyl N-(diphenylmethylene)glycinate (compound I-P, 18 g, 67.34 mmol) was added at this temperature in portions over 5 min. After aging 30 min, 2,2,2-trifluoro-1-iodoethane (14.5 g, 69.07 mmol) was added over 5 min, maintaining the temperature at −5° C. to 5° C. The reaction mixture was stirred at 0° C. for 6 h and then allowed to warm up to rt. After quenching by NH₄Cl, the mixture was extracted with EtOAc. The organic phase was washed with water, brine and dried with MgSO₄. After evaporation of the solvent, the crude product was purified by MPLC to give a colorless oil as the desired compound II-P (16.75 g, yield 71%). ¹H NMR (CDCl₃) δ: 7.69 (d, J=3.5 Hz, 2H), 7.54-7.36 (m, 6H), 7.30-7.28 (m, 2H), 4.48 (dd, J=3.5, 8.8 Hz, 1H), 4.30-4.20 (m, 2H), 2.99-2.86 (m, 2H), 1.32 (t, J=7.2 Hz, 3H).

Compound II-P (3.4 g, 9.73 mmol) was dissolved in 30 mL of EtOAc, and 10 mL of 3N HCl was added. The mixture was stirred at rt overnight. Solvent was removed under reduced pressure and the yellow solid was triturated with EtOAc a few times to give a white solid as the pure compound III-P (1.91 g, yield 88%). ¹H NMR (CD₃OD) δ: 4.72 (dd, J=4.8, 7.1 Hz, 1H), 4.36 (q, J=7.1 Hz, 2H), 3.10-3.02 (m, 1H), 2.96-2.88 (m, 1H), 1.36 (t, J=7.1 Hz, 3H).

Compound IV-P was prepared from compound III-P by the reductive alkylation of the amino acid similarly to the analogous step in the synthesis of intermediate A, with the exception that acetone is used instead of cyclopentanone. ¹H NMR (CDCl₃) δ: 4.21 (q, J=9.5 Hz, 2H), 3.59 (t, J=8.1 Hz, 1H), 2.75 (p, J=8.2 Hz, 1H), 2.56-2.35 (m, 2H), 1.28 (t, J=9.5 Hz, 3H), 1.01 (t, J=8.6 Hz, 6H).

The conversion of compound IV-P to Intermediate P-1 was similar to the conversion of compound III-M to Intermediate M-1 as described above. Intermediate P-1; ¹H NMR (CDCl₃) δ: 8.67 (s, 1H), 4.31-4.23 (m, 3H), 3.65 (p, J=6.5 Hz, 1H), 3.58-3.50 (m, 1H), 2.80-2.71 (m, 1H), 1.39 (d, J=6.5 Hz, 3H), 1.35 (d, J=6.5 Hz, 3H), 1.29 (t, J=7.1 Hz, 3H).

perdeuteroethyl 2-((2-chloro-5-nitropyrimidin-4-yl)(perdeuteroisopropyl)amino)butanoate (Intermediate Q-1)

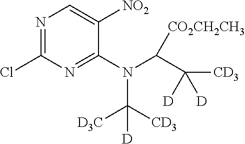

was prepared similarly, with perdeutero-iodoethane instead of 2,2,2-trifluoro-1-iodoethane; in the reductive alkylation of the amino acid, and with perdeutero-acetone instead of acetone and NaBD₃CN instead of sodium triacetoxyborohydride, and using CD₃OD as solvent.

### Methyl 1-(2-chloro-5-nitropyrimidin-4-yl)-2-ethylpiperidine-2-carboxylate (Intermediate Y-1)

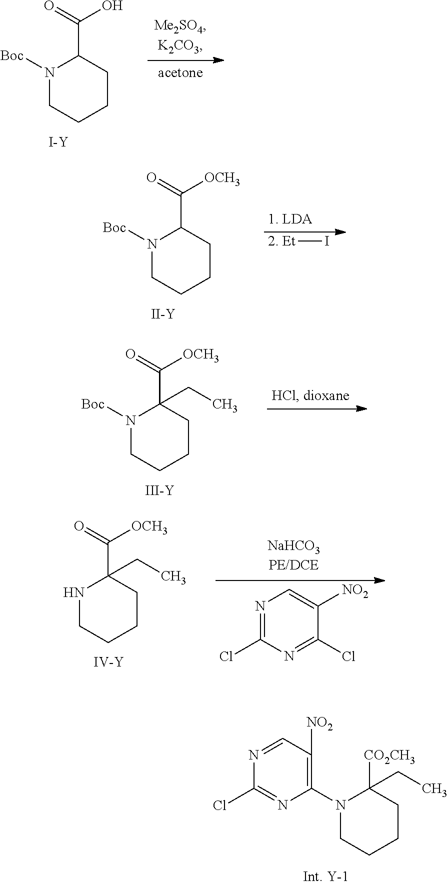

A 100 mL round bottom flask was charged with compound I-Y (5 g, 21.8 mmol), 40 mL of dry acetone, potassium carbonate (9 g, 69 mmol), and dimethylsulfate (3.8 mL, 38 mmol). A condenser was affixed, and the mixture was brought to reflux for 16 h. Upon cooling to 23° C., the reaction mixture was filtered to remove excess base, and the filtrate was concentrated under reduced pressure to give a clear oil as crude compound II-Y (2.25 g). LCMS: 266.1 m/z [M+Na], 144.1 m/z [M-Boc].

Crude compound II-Y (4.5 g, 18.5 mmol) was diluted with 6 mL of THF and slowly added at 0° C. to a preformed mixture of diisopropylamine (2.3 g, 23 mmol) and n-BuLi (10 mL of 2.3 M in THF) at 0° C. After stirring for 40 min at 0° C., a red color was observed, and ethyl iodide (2 mL, 25 mmol) was added by syringe as a neat liquid. After stirring for 0.5 h, the cooling bath was removed and the reaction slowly warmed to 23° C. over 16 h. The reaction mix was quenched by addition of saturated aqueous ammonium chloride and the biphasic mixture was extracted with EtOAc. The organic layer was rinsed with a saturated aqueous sodium bicarbonate solution, dried over sodium sulfate, and decanted before being concentrated under reduced pressure to give the desired compound III-Y. The compound was further purified by MPLC (0 to 100% EtOAc/hexane gradient) to give 3.8 g of compound III-Y.

Deprotection of compound III-Y was achieved by dissolving the pure material in 5 mL of DCM and adding 20 mL of 4N HCl in dioxane. After 1.5 h, LCMS confirmed complete formation of the amine. The reaction mix was concentrated under reduced pressure to give the HCl salt of compound IV-Y as a tan solid.

The conversion of compound IV-Y to Intermediate Y-1 was similar to the conversion of compound III-M to Intermediate M—as described above. Intermediate Y-1 (170 mg).

### Methyl 4-(2-chloro-5-nitropyrimidin-4-yl)-3-ethylmorpholine-3-carboxylate (Intermediate Z-1)

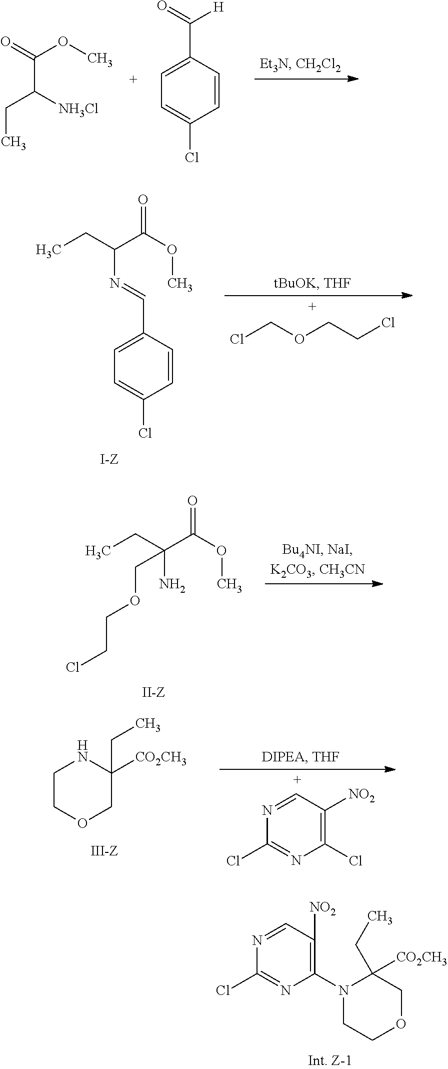

To a suspension of 2-amino-n-butyric acid methyl ester hydrochloride (73.71 mmol, 11.32 g) in 45 mL of DCM, triethylamine (36.85 mmol, 5.13 mL), and MgSO₄ (233.1 mmol, 28.06 g) were added. The suspension was stirred for 10 minutes before 4-chlorobenzaldehyde (36.85 mmol, 5.18 g) was added. The reaction mixture was stirred at rt under N₂ for 48 h, and then was filtered and concentrated. The resulting residue was dissolved in 50 mL of water and was washed with Et₂O (3×50 mL). The combined organic extracts were dried with MgSO₄, filtered and concentrated to provide compound I-Z.

The resulting residue (compound I-Z) was added to a −78° C. solution of potassium tert-butoxide (101.64 mmol, 11.41 g) in 50 mL of THF, and was stirred for 10 minutes before 1-chloro-2-(chloromethoxy)ethane (101.64 mmol, 13.11 g) was added. The reaction mixture was stirred for 18 h while slowly warming to rt. The temperature was then decreased to 0° C., and the reaction was quenched with 10 mL of water. The reaction mixture was stirred with 1N HCl at rt for 1.5 hours, and then was washed with 50 mL of Et₂O. The pH of the aqueous layer was adjusted to pH=8 with the addition of saturated K₂CO₃. The reaction mixture was extracted with DCM (3×50 mL). The combined organic extracts were dried with Na₂SO₄, filtered and concentrated to give compound II-Z.

The resulting residue (compound II-Z) was dissolved in 50 mL of acetonitrile and tetrabutyl ammonium iodide (1.477 mmol, 0.545 g), sodium iodide (73.87 mmol, 11.07 g), and K₂CO₃ (29.55 mmol, 4.08 g) were added. The reaction mixture was plunged into a preheated 90° C. oil bath and was stirred for 18 h. The reaction mixture was cooled to rt, filtered through a pad of Celite, and concentrated to give compound III-Z.

The conversion of compound III-Z to Intermediate Z-1 was similar to the conversion of compound III to Intermediate A as described above. Intermediate Z-1 (0.454 g, 4%); ¹H NMR (400 MHz, CDCl₃) δ: 8.78 (s, 1H), 3.91 (m, 5H), 3.72 (s, 3H), 3.56 (m, 1H), 3.04 (m, 1H), 2.50 (m, 1H), 1.97 (m, 1H), 0.86 (t, J=7.3 Hz, 3H), LCMS: 331.1 m/z (M+H)⁺; ret. Time: 1.724 min (Analytical Method A).

### (3R)-ethyl 2-(2-chloro-5-nitropyrimidin-4-yl)-2-azabicyclo[3.1.0]hexane-3-carboxylate (Intermediate AA)

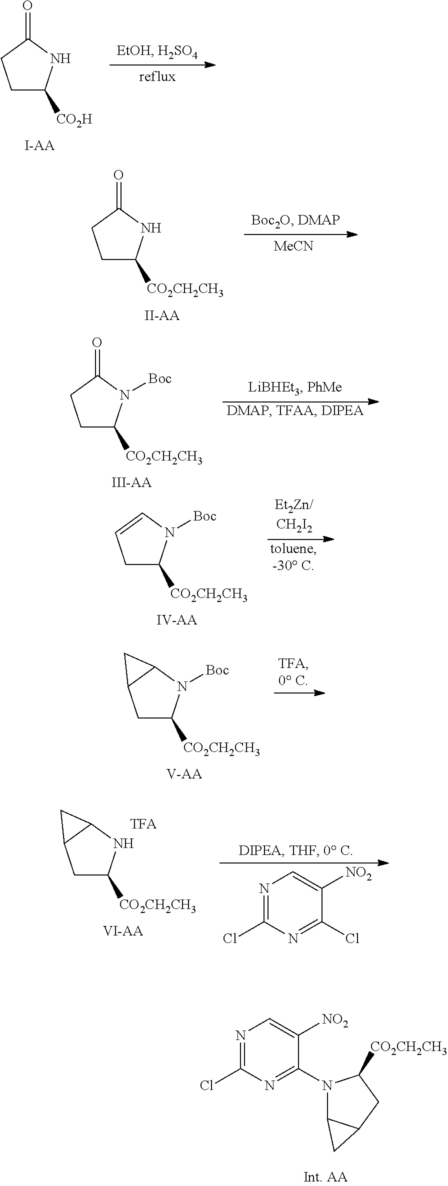

To a solution of D-pyroglutamic acid (compound I-A, 20.4 g, 0.16 mol) in 100 mL of EtOH, 1.2 mL of conc. sulfuric acid was added. The mixture was heated under reflux overnight. Solvent was removed under reduced pressure to give (R)-ethyl 5-oxopyrrolidine-2-carboxylate (compound II-AA).

To a solution of compound II-AA in 400 mL of acetonitrile cooled in an ice-bath, DMAP (2.65 g) and (Boc)₂O (51.8 g, 1.5 eq) were added. The mixture was stirred at rt overnight. Solvent was removed under reduced pressure and the resulting yellow oil was purified by MPLC to give 31 g of (R)-1-tert-butyl 2-ethyl 5-oxopyrrolidine-1,2-dicarboxylate (compound III-AA).

To a solution of compound III-AA (19.3 g, 75.2 mmol) in 162 mL of toluene at −78° C., LiBHEt₃ (82.7 mL, 1.0 M in THF) was added dropwise via syringe. The reaction mixture was stirred between −30 and −78° C. for 8 hours, followed by addition of DIPEA (73.3 mL), DMAP (915 mg) and TFAA (14.8 mL). The cooling bath was removed and the mixture was stirred at rt overnight. The reaction was quenched by water and diluted with 200 mL of EtOAc. The organic layer was separated and washed with water, brine and dried over MgSO₄. After evaporation of the solvent, the yellow oil was purified by MPLC to give 20.4 g of (R)-1-tert-butyl 2-ethyl 5-oxopyrrolidine-1,2-dicarboxylate (compound IV-AA). ¹H NMR (CDCl₃) δ: 6.53-6.65 (m, 1H), 4.96-4.91 (m, 1H), 4.67-4.55 (m, 1H), 4.24-4.17 (m, 2H), 3.13-3.01 (m, 1H), 2.71-2.61 (m, 1H), 1.74-1.49 (m, 9H), 1.31-1.26 (m, 3H). LCMS: 264.2 m/z (M+Na).

An oven-dried flask equipped with magnetic stirring bar was charged with 2.07 g (8.58 mmol) of compound IV-AA and 21 mL of dry toluene. The resulting solution was cooled to −30° C. and 15.6 mL of ZnEt₂ (1.1 M in toluene, 17.2 mmol) was added dropwise. A solution of 2.67 mL of diiodomethane (34.4 mmol) in 2.1 mL of toluene was then added to the mixture and the mixture was stirred between −25 and −30° C. for 6 hours. The reaction was quenched by adding 42 mL of 50% diluted sat. NaHCO₃. The organic layer was separated and the aqueous layer was extracted with EtOAc. The organic phases were combined and washed with water, brine and dried with MgSO₄. After evaporation of the solvent, the resulting yellow oil was purified by MPLC to give 2-tert-butyl 3-ethyl 2-azabicyclo[3.1.0]hexane-2,3-dicarboxylate (compound V-AA). LCMS: 278.1 m/z (M+Na); ret. Time 6.149 min (Analytical Method A).

Compound V-AA (515 mg, 2.02 mmol) was mixed with 1.5 mL of TFA and stirred at 0° C. for 30 min. TFA was removed under reduced pressure to give (3R)-ethyl 2-azabicyclo[3.1.0]hexane-3-carboxylate (compound VI-AA).

Compound VI-AA (2.17 mmol) was dissolved in 6 mL of THF and cooled to 0° C. DIPEA (1.05 mL, 3 eq) and 2,4-dichloro-4-nitropyrimidine (460 mg, 1.1 eq) were added sequentially. The mixture was stirred at 0° C. for 30 min. Thirty mL of EtOAc was added and the mixture was washed with sat NaHCO₃, water, brine and dried with MgSO₄. After evaporation of the solvent, the crude product was purified by MPLC to give pure (3R)-ethyl 2-(2-chloro-5-nitropyrimidin-4-yl)-2-azabicyclo[3.1.0]hexane-3-carboxylate (Intermediate AA). ¹H NMR (CDCl₃) δ: 8.60-8.54 (m, 1H), 5.23-5.20 (m, 0.67H), 4.68-4.66 (m, 0.33H), 4.21-4.09 (m, 2H), 3.30 (bs, 0.33H), 3.03 (bs, 0.33H), 2.83 (bs, 0.67H), 2.70-2.65 (m, 0.67H), 2.11-2.07 (m, 1H), 1.79-1.75 (m, 1H), 1.34-1.21 (m, 3H), 1.01 (bs, 1H), 0.82-0.79 (m, 1H).

### Methyl 2-((2-chloro-5-nitropyrimidin-4-yl)(3,3,3-trifluoropropyl)amino)-4,4,4-trifluorobutanoate (Intermediate BB-1)

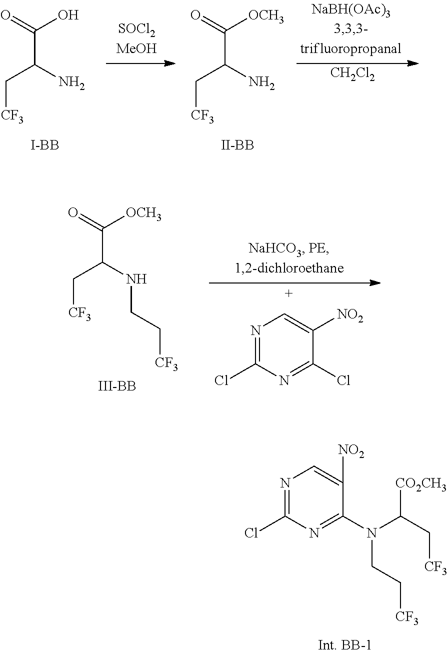

Compound I-BB (2 g, 12.73 mmol) was dissolved in 80 mL of methanol and cooled to 0° C. Thionyl chloride (1.66 mL, 22.91 mmol) was added dropwise over 20 minutes after which the reaction mixture was stirred at 70° C. for 3 h. The resulting solution was concentrated and dried under vacuum to give compound II-BB (2.14 g, 81%); LCMS: 172.0 m/z (M+H)⁺.

Compound II-BB (1.5 g, 7.22 mmol) and 3,3,3-trifluoropropanal (0.64 g, 5.79 mmol) were dissolved in 20 mL of DCM. After the addition of sodium acetate (0.59 g, 7.23 mmol) and sodium triacetoxyborohydride (2.0 g, 9.39 mmol), the mixture was stirred for 24 hr at rt and then saturated sodium bicarbonate solution was added. The aqueous phase was extracted with DCM. The combined organic phases were washed with water, dried over MgSO₄ and evaporated to give compound III-BB. LCMS: 268.1 m/z (M+H)⁺.

The conversion of compound III-BB to Intermediate BB-1 was similar to the conversion of compound III-J to Intermediate J-1 as described above. Intermediate BB-1 (2.14 g, 69%); LCMS: 425.0 m/z (M+H)⁺.

### Methyl 2-((2-chloro-5-nitropyrimidin-4-yl)(phenyl)amino)butanoate (Intermediate CC-1)

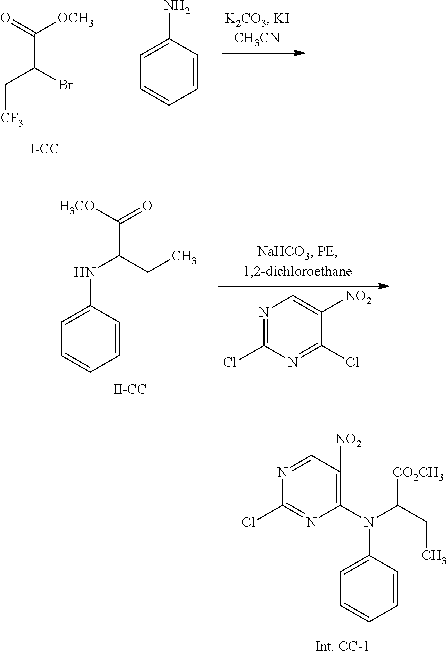

Compound I-CC (3.1 g, 17.1 mmol) and aniline (1.59 g, 17.1 mmol) were dissolved in 30 mL of acetonitrile in a glass pressure tube. After the addition of potassium carbonate (4.71 g, 34.2 mmol) and potassium iodide (0.283 g, 1.71 mmol), the tube was sealed and mixture was stirred for 18 hr at 100° C. The reaction mixture was diluted with ethyl acetate and washed with saturated sodium bicarbonate solution. The organic phase was dried over Na₂SO₄, filtered, evaporated down and purified by silica column (hexane:EtOAc) to give Compound II-CC (1.97 g, 59%); LCMS: 194.12 m/z (M+H)⁺.

The conversion of compound II-CC to Intermediate CC-1 was similar to the conversion of compound III-M to Intermediate M-1 as described above. Intermediate CC-1 (2.21 g, 62%); LCMS: 351.1 m/z (M+H)⁺.

Methyl 2-((2-chloro-5-nitropyrimidin-4-yl)(3-iodophenyl)amino)butanoate (Intermediate OO-0) and methyl 2-((2-chloro-5-nitropyrimidin-4-yl)(4-iodophenyl)amino)butanoate (Intermediate PP-1)

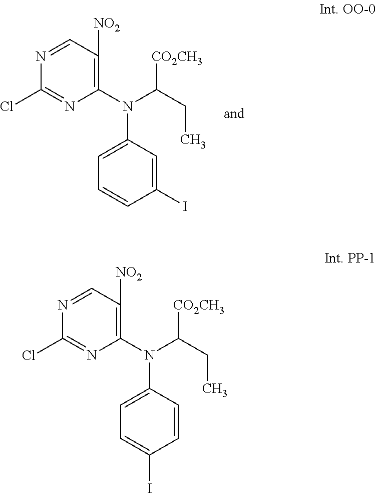

were prepared similarly with 3-iodoaniline and 4-iodoaniline, respectively, instead of aniline.

### (R)-methyl 2-((2-chloro-5-nitropyrimidin-4-yl)(cyclopropylmethyl)amino)butanoate (Intermediate DD-1)

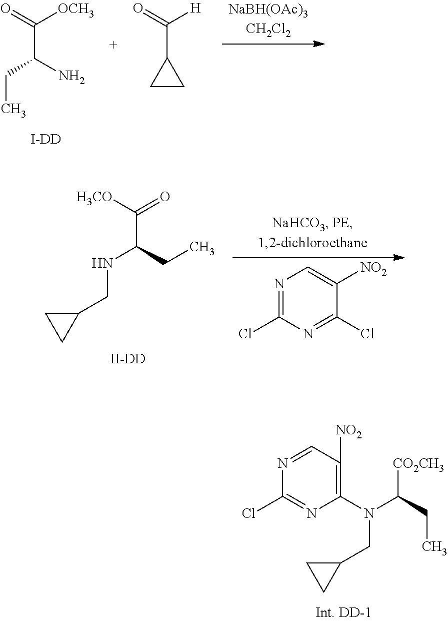

Compound I-DD (1.02 g, 6.70 mmol) and cyclopropanecarbaldehyde (0.375 g, 5.36 mmol) were dissolved in 10 mL of DCM. After the addition of sodium acetate (0.55 g, 5.36 mmol) and sodium triacetoxyborohydride (1.84 g, 8.71 mmol), the mixture was stirred for 18 hr at rt and then saturated sodium bicarbonate solution was added. The aqueous phase was extracted with DCM. The combined organic phases were washed with water, dried over MgSO₄ and evaporated down to give Compound II-DD; LCMS: 172.1 m/z (M+H)⁺.

The conversion of compound II-DD to Intermediate DD-1 was similar to the conversion of compound III-M to Intermediate M-1 as described above. Intermediate DD-1 (1.42 g, 65%); LCMS: 329.1 m/z (M+H)⁺.

### methyl 2-((2-chloro-5-nitropyrimidin-4-yl)(4-fluorophenyl)amino)butanoate (Intermediate EE-1)

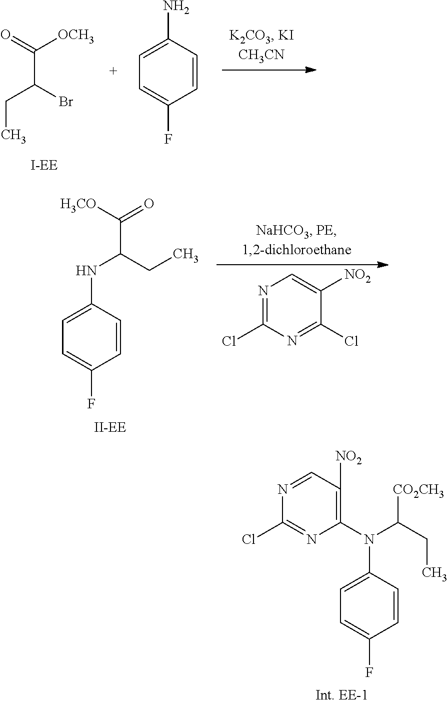

Compound I-EE (3.1 g, 17.1 mmol) and 4-fluoroaniline (1.90 g, 17.1 mmol) were dissolved in 30 mL of acetonitrile in a glass pressure tube. After the addition of potassium carconate (4.71 g, 34.2 mmol) and potassium iodide (0.283 g, 1.71 mmol), the tube was sealed and mixture was stirred for 18 hr at 100° C. The reaction mixture was diluted with ethyl acetate and washed with saturated sodium bicarbonate solution. The organic phase was dried over Na₂SO₄, filtered, evaporated down and purified by silica column (hexane:EtOAc) to give Compound II-EE (1.41 g, 39%); LCMS: 212.1 m/z (M+H)⁺.

The conversion of compound II-EE to Intermediate EE-1 was similar to the conversion of compound III-M to Intermediate M-1 as described above. Intermediate EE-1 (1.851 g, 79%); LCMS: 369.1 m/z (M+H)⁺.

Methyl 2-((2-chloro-5-nitropyrimidin-4-yl)(4-chlorophenyl)amino)butanoate (Intermediate TT-1) and (Intermediate UU-1)

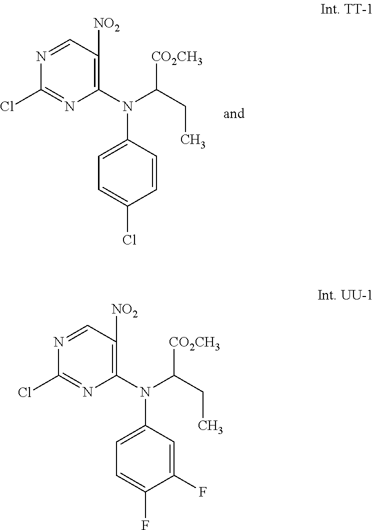

were prepared similarly, with 4-chloroaniline or 3,4-difluoroaniline, respectively, instead of 4-fluoroaniline.

### (2R)-methyl 2-((3-(benzyloxy)cyclopentyl) (2-chloro-5-nitropyrimidin-4-yl)amino)butanoate (Intermediate FF-1)

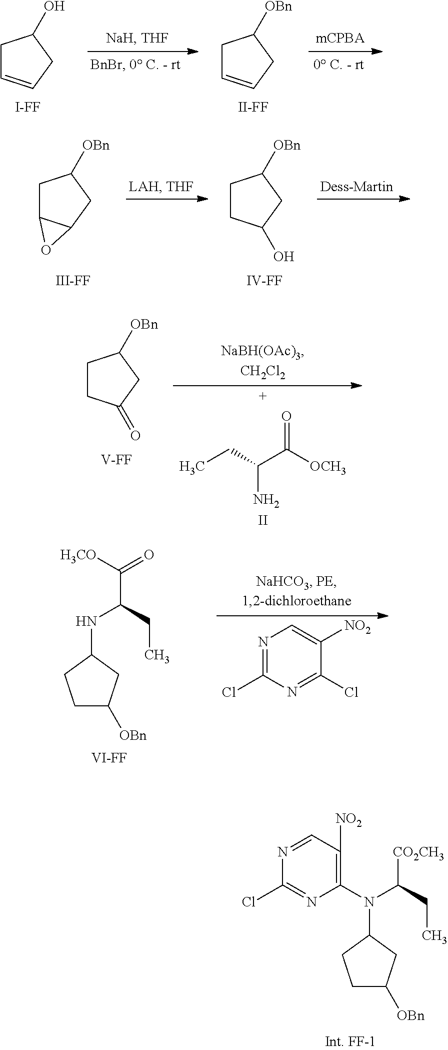

To a stirring mixture of cyclopent-3-enol (1-FF, 2.4 g, 28.5 mmol) in 41 mL of THF at 0° C., NaH (1.6 g, 39.9 mmole, 60% in mineral oil) was added portion wise. The reaction mixture was warmed to rt for 15 min. The reaction mixture was cooled to 0° C. before BnBr was added. The reaction mixture was stirred for 4 h before it was slowly quenched with water and the resulting mixture was diluted with 100 mL of EtOAc. The layers were separated. The aqueous layer was extracted with EtOAc (2×50 mL). The combined organic layers were dried over MgSO₄, filtered, and concentrated under reduced pressure. The crude product was purified by MPLC, using EtOAc/Hex to give compound II-FF (1.2 g). LCMS: 175.1 m/z (M+H)⁺.

To a stirring mixture of ((cyclopent-3-enyloxy)methyl)benzene (II-FF, 1.2 g, 6.85 mmol) in DCM at 0° C., mCPBA (0.13 g, 7.58 mmol) was added in one portion. The reaction mixture was stirred at 0° C. for 2 h before it was slowly warmed to rt. The reaction mixture was slowly quenched with a saturated NaHSO₃ and NaHCO₃ solution (1:1, 10 mL). The reaction was diluted with EtOAc. The layers were separated. The aqueous layer was extracted with EtOAc (2×50 mL). The combined organic layers were dried over MgSO₄, filtered, and concentrated under reduced pressure. The crude product was purified by MPLC, using EtOAc/Hex to give compound III-FF (1.1 g). LCMS: 191.1 m/z (M+H)⁺.

To a stirring mixture of the epoxide (III-FF, 1.1 g, 5.75 mmol) in 10 mL of THF at 0° C., a solution of LiAlH (6.4 mL, 6.36 mmol, 1.0 M in THF) was added dropwise. The reaction mixture was stirred for 2 h at 0° C. and quickly warmed to rt for 5 min. To this a mixture of Celite/Na₂SO₄.10H₂O (1:1, 5 g total) was added until all the gas was evolved. The solid mixture was dissolved in ether and filtered through a plug of Celite to give the desired compound IV-FF. LCMS: 193.2 m/z (M+H)⁺.

To a stirring mixture of 3-(benzyloxy)cyclopentanol (IV-FF, 1.7 g) in 30 mL of DCM, NaHCO₃ (3.7 g, 44 mmole), and Dess Martin reagent (11.2 g, 26.42 mmol) were added. The resulting mixture was stirred at rt until all the alcohols were consumed. The reaction mixture was slowly quenched with a saturated NaHSO₃ and NaHCO₃ solution (1:1, 20 mL total volume). The reaction mixture was diluted with EtOAc. The layers were separated and the aqueous layer was extracted with EtOAc (2×50 mL). The combined organic layers were dried over MgSO₄, filtered, and concentrated under reduced pressure. The crude product was purified by MPLC, using EtOAc/Hex, to give compound V-F (1.4 g). LCMS: 191.2 m/z (M+H)⁺.

To a stirring mixture of compound II (500 mg, 3.26 mmol, prepared as in synthesis of Intermediate A) and 3-(benzyloxy)cyclopentanone (V-F, 622 mg, 3.26 mmol) in 7 mL of DCM, sodium acetate (350 mg, 4.3 mmol) and sodium triacetoxyborohydride (1.0 g, 4.56 mmol) were added at 0° C. The resulting mixture was stirred for 12 hr at rt and 50 mL of a saturated sodium bicarbonate solution was added. The layers were separated and the aqueous phase was extracted with DCM (2×25 mL). The combined organic phases were washed with water, dried over MgSO₄ and evaporated under reduced pressure to give compound VI-F. LCMS: 292.3 m/z (M+H)⁺.

The conversion of compound VI-FF to Intermediate FF-1 was similar to the conversion of compound III to Intermediate A as described above. Intermediate FF-1; LCMS: 449.3 m/z (M+H)⁺.

### (+/−)Ethyl 1-(2-chloro-5-nitropyrimidin-4-yl)-2-(2,2,2-trifluoroethyl)pyrrolidine-2-carboxylate (Intermediate II)

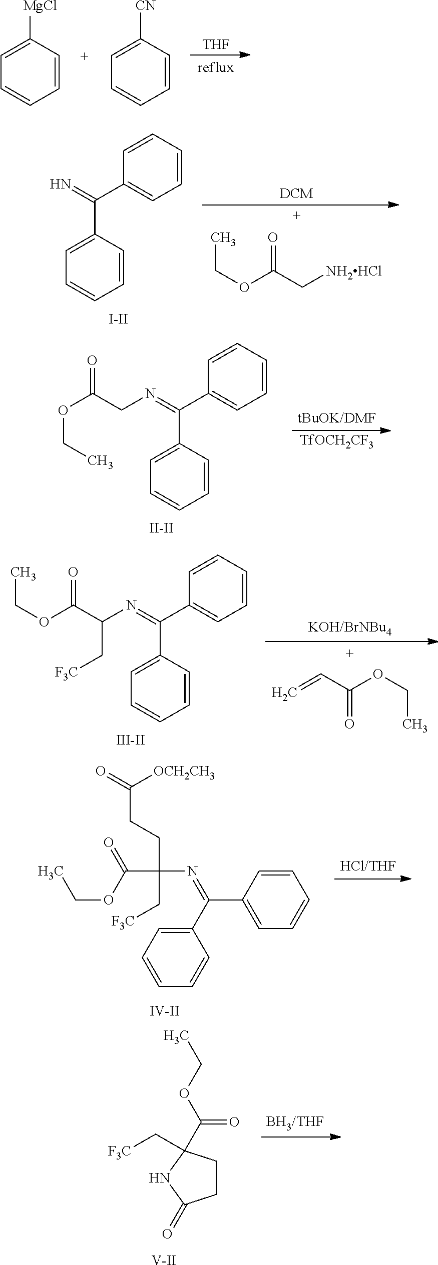

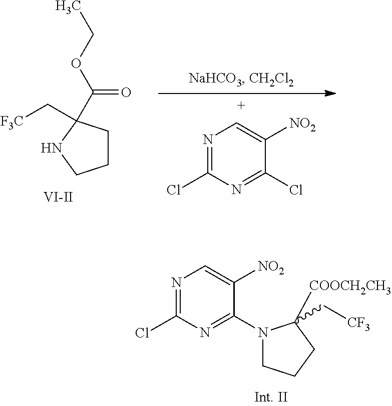

To a solution of phenylmagnesium chloride (100 ml, 200 mmol) in 100 mL of THF, benzonitrile (20.6 g, 200 mmol) was added at 0° C. The mixture was refluxed for 4 h, and then cooled to 0° C. Dry methanol (200 ml) was added carefully, and the solvent was evaporated to give compound I-II. LCMS: 182.1 m/z (M+H)⁺.

A mixture of compound I-II (36.2 g, 200 mmol), ethyl 2-aminoacetate (28 g, 200 mmol) and 500 mL of DCM was stirred overnight at rt, filtered and the filtrate was washed with water (2×400 mL), dried with Na₂SO₄, concentrated and the residue was crystallized from PE to give compound II-II. LCMS: 268.1 m/z (M+H)⁺.

To a solution of t-BuOK (4.41 g, 39.3 mmol) in 30 mL of dry, compound II-II (10 g, 37.4 mmol, dissolved in 20 mL dry DMF) was added at 0° C. over 10 min. After 30 min, TfOCH₂CF₃ (10.1 g, 43.4 mmol) was added at 0° C. over 10 min, then the mixture was stirred at rt 18 h. The mixture was partitioned between 5% aqueous NH₄Cl and EtOAc, and the organic layer was washed by saturated aqueous NaCl, dried over Na₂SO₄, concentrated under reduced pressure, and purified by chromatography (PE:EtOAc=15:1) to give compound III-II. LCMS: 350.1 m/z (M+H)⁺.

To a solution of KOH (5.0 g, 88.5 mmol) and BrNBu₄ (0.95 g, 2.95 mmol) in 60 mL of CH₃CN, a solution of compound III-II (10.3 g, 29.5 mmol) and ethyl acrylate (14.8 g, 147.6 mmol) in 60 mL of CH₃CN was added dropwise at rt. The mixture was stirred 18 h and then the solvent was removed under vacuum. The residue was dissolved in 200 mL of diethyl ether, and washed with water (3×200 mL), dried over Na₂SO₄, evaporated and purified by chromatography (PE:EtOAc=10:1) to give compound IV-II. LCMS: 450.1 m/z (M+H)⁺.

A mixture of compound IV-II (7.33 g, 16.3 mmol), 3 mL of concentrated HCl and 50 mL of THF was heated at 40° C. overnight. The solvent was removed and the residue was partitioned between water and EtOAc. The organic layer was washed with water (2×100 mL), dried over Na₂SO₄, evaporated and purified by flash silica column (PE:EtOAc=75%:25%) to give compound V-II. LCMS: 240.1 m/z (M+H)⁺.

To compound V-II (1.21 g, 5.06 mmol) in 15 mL of THF, BH₃ (1M in THF, 10.1 ml, 10.1 mmol) was carefully added at 0° C. and the mixture was stirred overnight at rt. Ten mL of 1N HCl was added to quench the reaction, then adjusted to pH 7 with aqueous NH₄OH. The mixture was concentrated and extracted with 75 mL of EtOAc and the organic layer was washed with water (2×50 mL), dried over Na₂SO₄ and evaporated to give compound VI-II. LCMS: 226.1 m/z (M+H)⁺.

Compound VI-II (595 mg, 2.64 mmol), 2,4-dichloro-5-nitropyrimidine (615 mg, 3.17 mmol), NaHCO₃ (444 mg, 5.29 mmol) and 20 mL of DCM were stirred at rt for 18 h. The reaction was filtered and the filtrate was washed with water (2×25 mL), dried over Na₂SO₄ and evaporated, then purified by flash silica column (PE: EtOAc=60%:40%) to give Intermediate II. LCMS: 383.1 m/z (M+H)⁺.

### 1-tert-butyl 3-methyl 4-(2-chloro-5-nitropyrimidin-4-yl)-3-ethylpiperazine-1,3-dicarboxylate (Intermediate JJ-1)

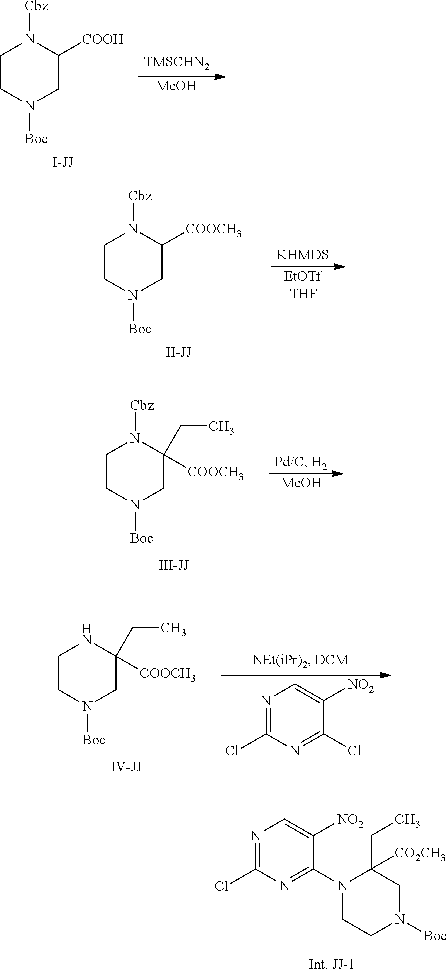

1-(Benzyloxycarbonyl)-4-(tert-butoxycarbonyl)piperazine-2-carboxylic acid (I-JJ, 1.07 g, 2.9 mmol, Small Molecules, Inc., Hoboken, N.J. USA) was dissolved in 10 mL of dry methanol and trimethylsilyl diazomethane (2.0 M in diethyl ether, Aldrich) was added dropwise with stirring at rt until a slight yellow color persisted. The solution was then concentrated under reduced pressure, and flash chromatography (0-50% EtOAc/hexanes elution) gave 1-benzyl 4-tert-butyl 2-methyl piperazine-1,2,4-tricarboxylate (compound II-JJ) as a colorless oil: [M+Na]⁺=401.2 (35); [M-Boc+H]⁺=279.1 (100).

Following the procedure according to WO 2005/079799 (the disclosure of which is hereby incorporated by reference with respect to this synthesis), 1-benzyl 4-tert-butyl 2-methyl piperazine-1,2,4-tricarboxylate (II-JJ, 1.1 g, 2.9 mmol) was dissolved in 6 mL of dry THF and cooled to −78° C. Potassium hexamethyldisilazane (0.5M solution in toluene, Aldrich, 10 mL, 5.0 mmol) was added by syringe, and the reaction mixture stirred at −78° C. for 75 min. Ethyl trifluoromethanesulfonate (0.65 mL, 5.0 mmol) was added dropwise by syringe to this mixture, and then the reaction was allowed to warm to rt for 5 h. The reaction was quenched with saturated sodium bicarbonate solution, and the mixture was extracted twice with ethyl acetate. The combined organics were dried with MgSO₄, filtered and concentrated under reduced pressure. Flash chromatography (0-10% methanol/DCM gradient elution) gave 1-Benzyl 4-tert-butyl 2-methyl 2-ethylpiperazine-1,2,4-tricarboxylate (compound III-JJ) as a yellow oil, approximately 5:1 ratio of methyl and ethyl esters (1.06 g): LCMS: [M+Na]⁺=429.2 (60); [M-Boc+H]⁺=307.1 (100).

1-Benzyl 4-tert-butyl 2-methyl 2-ethylpiperazine-1,2,4-tricarboxylate (1.1 g, 2.7 mmol) was dissolved in 10 mL of methanol and glacial acetic acid (2 drops) was added. Palladium on carbon (5%, 410 mg) was added, and the reaction mixture was stirred under a H₂ atmosphere for 17 h at rt. The mixture was filtered through diatomaceous earth and the filter cake washed with MeOH. The combined filtrates were concentrated under reduced pressure to give 1-tert-butyl 3-methyl 3-ethylpiperazine-1,3-dicarboxylate (compound IV-JJ) as an oil. LCMS: 273.1 m/z (M+H)⁺.

The conversion of compound IV-JJ to Intermediate JJ-1 was similar to the conversion of compound III to Intermediate A as described above. Intermediate JJ-1; LCMS: 430.1 m/z (M+H)⁺.

### Methyl 2-((2-chloro-5-nitropyrimidin-4-yl)(1-((2-(trimethylsilyl)ethoxy)methyl)-1H-pyrazol-4-yl)amino)butanoate (Intermediate KK-1)

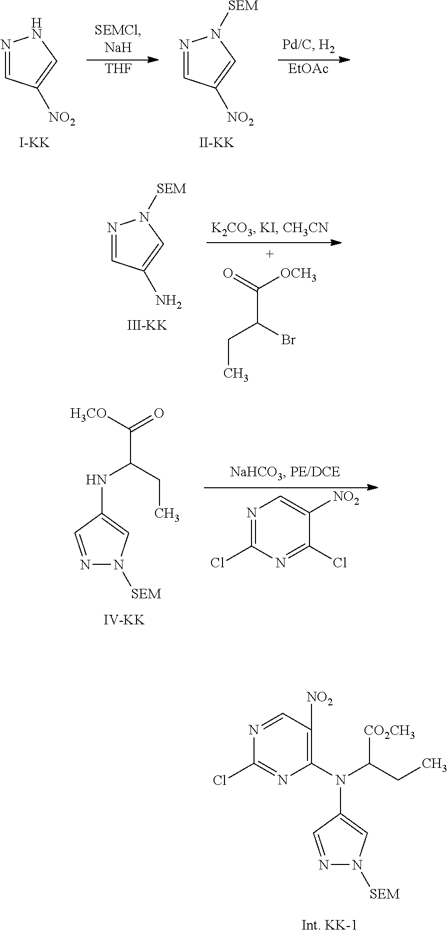

Sodium hydride (849 mg of a 60% dispersion in mineral oil, 21.2 mmol) was added to a solution of compound I-KK ((2 g, 17.7 mmol) in 80 mL of THF at 0° C. and the resulting mixture was stirred for 10 minutes. SEM-Cl (3.43 mL, 19.5 mmol) was added dropwise and the resulting mixture was stirred at rt for 1 h. The reaction mixture was diluted with ethyl acetate and washed with brine. The organic phase was dried over Na₂SO₄ and evaporated. The residue was purified by silica column (hexane:EtOAc) to give compound II-KK (4.01 g, 93%); LCMS: 243.8 m/z (M+H)⁺.

Palladium on carbon (10%, 0.5 g) was added to a solution of compound II-KK (4.01 g, 16.4 mmol) in 50 mL of ethyl acetate and the resulting suspension was stirred under 1 atm of hydrogen for 2 hr. The mixture was filtered through a pad of Celite and the filtrate was concentrated under vacuum to give compound III-KK (3.24 g, 93%); LCMS: 214.1 m/z (M+H)⁺.

Compound III-KK (1.21 g, 5.67 mmol) and methyl 2-bromobutanoate (1.54 g, 8.51 mmol) were dissolved in 15 mL of acetonitrile in a glass pressure tube. Potassium carbonate (1.56 g, 11.342 mmol) and potassium iodide (94 mg, 0.567 mmol) were added and the tube was sealed and the mixture was stirred for 18 hr at 100° C. The reaction mixture was diluted with ethyl acetate and washed with saturated sodium bicarbonate solution. The organic phase was dried over Na₂SO₄, filtered, concentrated and purified by silica column (hexane:EtOAc) to give compound IV-KK (1.42 g, 79%); LCMS: 314.1 m/z (M+H)⁺.

The conversion of compound IV-KK to Intermediate KK-1 was similar to the conversion of compound III-M to Intermediate M-1 as described above. Intermediate KK-1 (1.83 g, 86%); LCMS: 471.2 m/z (M+H)⁺.

Methyl 2-((2-chloro-5-nitropyrimidin-4-yl)(1-((2-(trimethylsilyl)ethoxy)methyl)-1H-pyrazol-3-yl)amino)butanoate (Intermediate QQ-1)

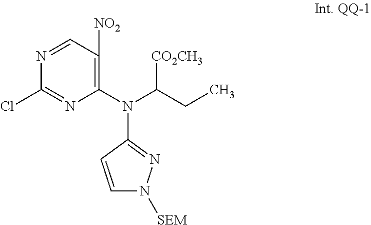

was prepared similarly, with 3-nitro-1H-pyrazole instead of 4-nitro-1H-pyrazole. Intermediate QQ-1 (0.624 g, 26%); LCMS: 471.2 m/z (M+H)⁺.

### (S)-methyl 1-(2-chloro-5-nitropyrimidin-4-yl)-2-ethylpyrrolidine-2-carboxylate (Intermediate XX-1)

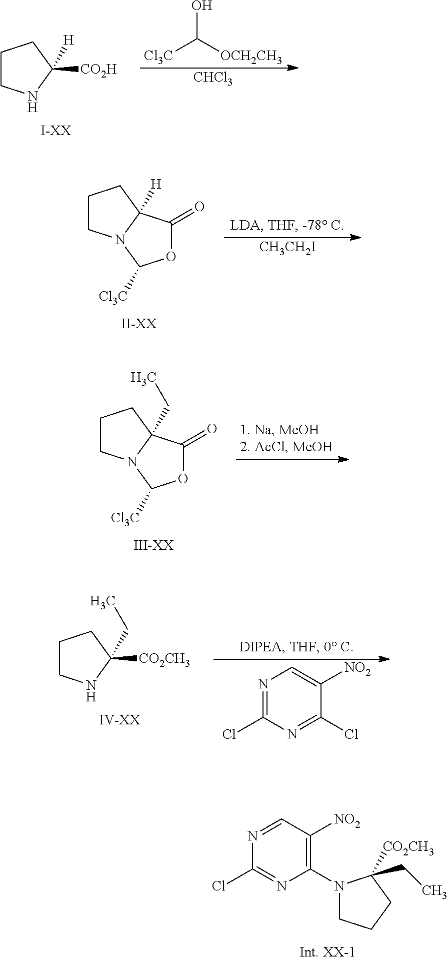

To a suspension of Compound I-XX (11.55 g, 100.3 mmol) in 500 mL of chloroform, 2,2,2-trichloro-1-ethoxyethanol (23.27 g, 120.3 mmol) was added. The reaction flask was fitted with a 25-mL Dean-Stark trap and reflux condenser, and the reaction mixture was heated to reflux for 18 h. The reaction mix was cooled to rt and the volatile organics were removed under reduced pressure. The resulting residue was recrystallized from EtOH, by dissolving the residue in 30 mL of boiling EtOH, pouring the hot solution into a 125-mL Erlenmeyer flask, slowly cooling the flask to rt, and then cooling to 0° C. for 1 h. The resulting crystals were isolated by filtration and washed with cold EtOH to provide compound II-XX (15.19 g, 62%).

To a solution of N,N-diisopropylamine (7.94 mL, 56.18 mmol) in 25 mL of THF at −78° C., n-butyllitium in hexanes (1.6 M, 37.62 mL, 60.19 mmol) was added. The reaction mixture was stirred for 30 minutes at −78° C., then was warmed to 0° C. for 30 minutes. The reaction was cooled to −78° C. and a solution of compound II-XX (9.75 g, 40.13 mmol) in 50 mL of THF was added rapidly via addition funnel. The reaction mixture was stirred for 30 minutes at −78° C. Iodoethane (5.83 mL, 72.23 mmol) was added via syringe in a single portion. The reaction mixture was warmed to −40° C. and was stirred for 1 h. The reaction mixture was poured into a reparatory funnel containing 200 mL of water and was extracted with chloroform (3×300 mL). The combined organic extracts were dried with anhydrous Na₂SO₄, filtered and concentrated to provide compound III-XX (10.94 g, 71%).

Compound III-XX (29.0 mmol, 7.90 g) was dissolved in 75 mL of MeOH and sodium (0.420 g, 18.3 mmol) was added in small pieces. The reaction mixture was stirred for 30 minutes at rt until all of the sodium dissolved. The temperature was decreased to 0° C., and acetyl chloride (40 mL, 563 mmol) was added slowly via addition funnel (˜1 drop/sec). Upon complete addition of the acetyl chloride, the reaction mixture was warmed to rt and then transferred to a preheated 65° C. oil bath. The reaction mixture was stirred at 65° C. for 12 h, and then was cooled to rt. The reaction mixture was concentrated and the resulting residue was purified by flash chromatography (10% MeOH in CH₂Cl₂, stains bright yellow in KMnO₄ (R_{f}: 0.29, 10% MeOH in CH₂Cl₂)) to provide compound IV-XX (3.28 g, 59%).

The conversion of compound IV-XX to Intermediate XX-1 was similar to the conversion of compound III to Intermediate A as described above. Intermediate XX-1 (6.16 g, 61%).

(S)-methyl 1-(2-chloro-5-nitropyrimidin-4-yl)-2-perdeuteroethyl-pyrrolidine-2-carboxylate (Intermediate S-1) and (S)-methyl 1-(2-chloro-5-nitropyrimidin-4-yl)-2-methylpyrrolidine-2-carboxylate (Intermediate T-1),

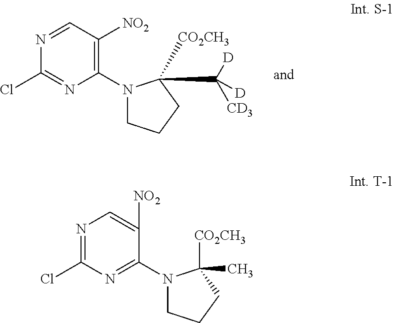

were prepared similarly, with perdeuteroiodoethane or iodomethane, respectively, instead of iodoethane.

### (R)-2-Chloro-8-cyclobutyl-7-ethyl-7,8-dihydropteridin-6(5H)-one (Intermediate B)

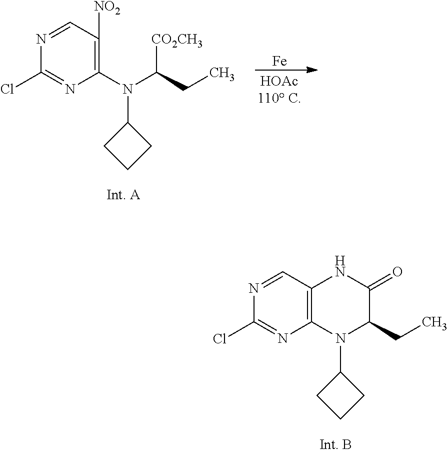

Intermediate A (1.1 g, 1 EQ) in HOAc (5 mL) was stirred and iron powder (1.87 g, 6 EQ) was added. The reaction was heated at 100° C. for 1 h. The reaction mixture was filtered hot and the cake was further purified with HOAc. The mother liquors were concentrated under reduced pressure. The residue was taken up with 3 N NaOH and EtOAc. The layers were separated and the aqueous layer was extracted with EtOAc. The crude product mixture was further purified via the isco column to give the desired (R)-2-chloro-8-cyclobutyl-7-ethyl-7,8-dihydropteridin-6(5H)-one (Intermediate B, 680 mg, 76% yield). LC-MS: [M+H] 267.1.

The cyclization can alternatively be done using Raney nickel and hydrogen, as was done with Intermediate E-0 as follows:

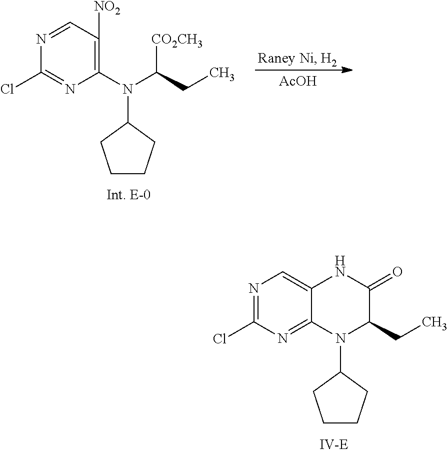

Intermediate E-0 (1 g) was dissolved in AcOH (5 ml), Raney Ni (400 mg) was added, and the mixture was stirred under H₂ at 50° C. until Intermediate E-0 was consumed. The solvent was removed by evaporation under vacuum, and the residue was purified by flash silica column to give (R)-2-chloro-8-cyclopentyl-7-ethyl-7,8-dihydropteridin-6(5H)-one (compound IV-E, 530 mg, yield 65%).

### (R)-7-Chloro-5-cyclobutyl-4-ethyl-4,5-dihydro-[1,2,4]-triazolo[4,3-f]pteridine (Intermediate C)

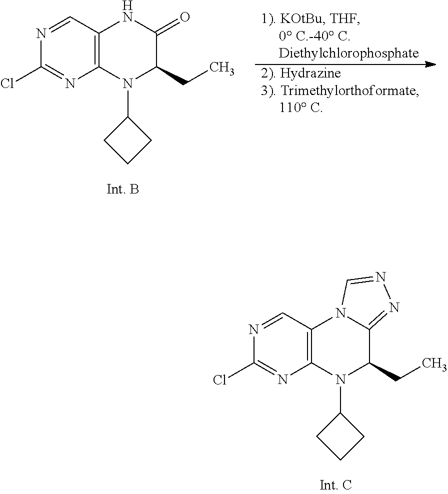

Intermediate B ((R)-2-chloro-8-cyclobutyl-7-ethyl-7,8-dihydropteridin-6(5H)-one, 440 mg, 1 EQ) in THF (8 mL) was stirred at −20° C. and potassium tert-butoxide (240 mg, 1.3 EQ) was added over 5 min. The reaction mixture was warmed up to 0° C. for 25 min after complete addition. The reaction mixture was cooled to −40° C. and diethylchlorophosphate (400 mg, 1.4 EQ) was added. The reaction mixture was warmed up to rt for 45 min. To the resulting mixture 1M hydrazine (10 EQ) was added and the reaction mixture was stirred at rt for 18 h. The reaction mixture was concentrated under reduced pressure and diluted with DCM and a saturated NaHCO₃ solution. The organic layer was dried over MgSO₄ and concentrated under pressure. The crude material was purified via the iso column. LC-MS: [M+H] 281.1. The resulting material was dissolved in trimethylorthoformate (10 EQ) and heated to 110° C. for 1 h. The reaction mixture was concentrated under reduced pressure and purified via silica gel column chromatography to afford the desired (R)-7-Chloro-5-cyclobutyl-4-ethyl-4,5-dihydro-[1,2,4]triazolo[4,3-f]pteridine (Intermediate C) as a white solid. LC-MS: [M+H] 291.1.

### (R)-7-Chloro-5-cyclobutyl-4-ethyl-1-methyl-4,5-dihydro-[1,2,4]-triazolo[4,3-f]pteridine (Intermediate D)

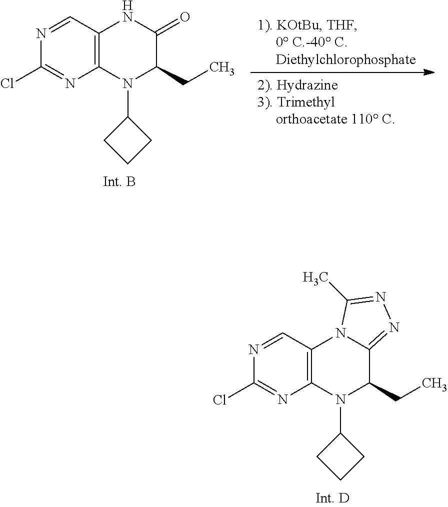

Intermediate D was prepared in the same manner as intermediate C, using trimethyl orthoacetate instead of trimethyl orthoformate in the last step.

Additional intermediates are prepared similarly to the preparation of Intermediates C or D, using a suitable Intermediate instead of Intermediate A. The initial cyclization step in some instances is performed similarly to the reaction described for Intermediate E-0. The following compounds are prepared:
(R)-7-chloro-5-cyclopentyl-4-ethyl-4,5-dihydro-[1,2,4]triazolo[4,3-f]pteridine (Intermediate E),
(R)-7-chloro-5-cyclopentyl-4-ethyl-1-methyl-4,5-dihydro-[1,2,4]triazolo[4,3-f]pteridine (Intermediate F),
(R)-7-chloro-4-ethyl-5-isopropyl-4,5-dihydro-[1,2,4]triazolo[4,3-f]pteridine (Intermediate G), and
(R)-7-chloro-4-ethyl-5-isopropyl-1-methyl-4,5-dihydro-[1,2,4]triazolo[4,3-f]pteridine (Intermediate H),
(R)-7-chloro-11,12,13,13a-tetrahydro-10H-pyrido[2,1-h][1,2,4]triazolo[4,3-f]pteridine (Intermediate I),
(R)-7-chloro-3-methyl-11,12,13,13a-tetrahydro-10H-pyrido[2,1-h][1,2,4]triazolo[4,3-f]pteridine (Intermediate J),
(R)-7-chloro-10,11,12,12a-tetrahydropyrrolo[2,1-h][1,2,4]triazolo[4,3-f]pteridine (Intermediate K), and
(R)-7-chloro-3-methyl-10,11,12,12a-tetrahydropyrrolo[2,1-h][1,2,4]triazolo[4,3-f]pteridine (Intermediate L),
(R)-7-chloro-4-ethyl-5-(tetrahydro-2H-pyran-4-yl)-4,5-dihydro-[1,2,4]triazolo[4,3-f]pteridine (Intermediate M),
(R)-7-chloro-4-ethyl-1-methyl-5-(tetrahydro-2H-pyran-4-yl)-4,5-dihydro-[1,2,4]triazolo[4,3-f]pteridine (Intermediate M′),
(R)-7-chloro-4-ethyl-5-(tetrahydrofuran-3-yl)-4,5-dihydro-[1,2,4]triazolo[4,3-f]pteridine (Intermediate N),
(R)-7-chloro-4-ethyl-1-methyl-5-(tetrahydrofuran-3-yl)-4,5-dihydro-[1,2,4]triazolo[4,3-f]pteridine (Intermediate N′),
(R)-7-chloro-5-cyclopropyl-4-ethyl-4,5-dihydro-[1,2,4]triazolo[4,3-f]pteridine (Intermediate O),
(R)-7-chloro-5-cyclopropyl-4-ethyl-1-methyl-4,5-dihydro-[1,2,4]triazolo[4,3-f]pteridine (Intermediate O′),
7-chloro-5-isopropyl-4-(2,2,2-trifluoroethyl)-4,5-dihydro-[1,2,4]triazolo[4,3-f]pteridine (Intermediate P),
7-chloro-5-isopropyl-1-methyl-4-(2,2,2-trifluoroethyl)-4,5-dihydro-[1,2,4]triazolo[4,3-f]pteridine (Intermediate P′),
(R)-7-chloro-4-perdeuteroethyl-5-perdeuteroisopropyl-4,5-dihydro-[1,2,4]triazolo[4,3-f]pteridine (Intermediate Q), and
(R)-7-chloro-4-perdeuteroethyl-5-perdeuteroisopropyl-1-methyl-4,5-dihydro-[1,2,4]triazolo[4,3-f]pteridine (Intermediate Q′),
7-chloro-5-isopropyl-5H-spiro[[1,2,4]triazolo[4,3-f]pteridine-4,1′-cyclopropane] (Intermediate R),
7-chloro-5-isopropyl-1-methyl-5H-spiro[[1,2,4]triazolo[4,3-f]pteridine-4,1′-cyclopropane] (Intermediate R′),
(S)-7-chloro-12a-perdeuteroethyl-10,11,12,12a-tetrahydropyrrolo[2,1-h][1,2,4]triazolo[4,3-f]pteridine (Intermediate S),
(S)-7-chloro-12a-perdeuteroethyl-3-methyl-10,11,12,12a-tetrahydropyrrolo[2,1-h][1,2,4]triazolo[4,3-f]pteridine (Intermediate S′),
(S)-7-chloro-12a-methyl-10,11,12,12a-tetrahydropyrrolo[2,1-h][1,2,4]triazolo[4,3-f]pteridine (Intermediate T),
(S)-7-chloro-3,12a-dimethyl-10,11,12,12a-tetrahydropyrrolo[2,1-h][1,2,4]triazolo[4,3-f]pteridine (Intermediate T′),
(R)-7-chloro-4-ethyl-5-(3,3,3-trifluoropropyl)-4,5-dihydro-[1,2,4]triazolo[4,3-f]pteridine (Intermediate U),
(R)-7-chloro-4-ethyl-1-methyl-5-(3,3,3-trifluoropropyl)-4,5-dihydro-[1,2,4]triazolo[4,3-f]pteridine (Intermediate U-1),
(R)-5-(3-(benzyloxy)cyclobutyl)-7-chloro-4-ethyl-4,5-dihydro-[1,2,4]triazolo[4,3-f]pteridine (Intermediate V-2),
(R)-5-(3-(benzyloxy)cyclobutyl)-7-chloro-4-ethyl-1-methyl-4,5-dihydro-[1,2,4]triazolo[4,3-f]pteridine (Intermediate V′-2),
7-chloro-13a-ethyl-11,12,13,13a-tetrahydro-10H-pyrido[2,1-h][1,2,4]triazolo[4,3-f]pteridine (Intermediate Y),
7-chloro-13a-ethyl-3-methyl-11,12,13,13a-tetrahydro-10H-pyrido[2,1-h][1,2,4]triazolo[4,3-f]pteridine (Intermediate Y′),
7-chloro-13a-ethyl-10,11,13,13a-tetrahydro-[1,4]oxazino[3,4-h][1,2,4]triazolo[4,3-f]pteridine (Intermediate Z),
7-chloro-13a-ethyl-3-methyl-10,11,13,13a-tetrahydro-[1,4]oxazino[3,4-h][1,2,4]triazolo[4,3-f]pteridine (Intermediate Z′),
7-chloro-4-(2,2,2-trifluoroethyl)-5-(3,3,3-trifluoropropyl)-4,5-dihydro-[1,2,4]triazolo[4,3-f]pteridine (Intermediate BB),
7-chloro-1-methyl-4-(2,2,2-trifluoroethyl)-5-(3,3,3-trifluoropropyl)-4,5-dihydro-[1,2,4]triazolo[4,3-f]pteridine (Intermediate BB′),
7-chloro-4-ethyl-5-phenyl-4,5-dihydro-[1,2,4]triazolo[4,3-f]pteridine (Intermediate CC),
7-chloro-4-ethyl-1-methyl-5-phenyl-4,5-dihydro-[1,2,4]triazolo[4,3-f]pteridine (Intermediate CC′),
(R)-7-chloro-5-(cyclopropylmethyl)-4-ethyl-4,5-dihydro-[1,2,4]triazolo[4,3-f]pteridine (Intermediate DD),
(R)-7-chloro-5-(cyclopropylmethyl)-4-ethyl-1-methyl-4,5-dihydro-[1,2,4]triazolo[4,3-f]pteridine (Intermediate DD′),
7-chloro-4-ethyl-5-(4-fluorophenyl)-4,5-dihydro-[1,2,4]triazolo[4,3-f]pteridine (Intermediate EE),
7-chloro-4-ethyl-5-(4-fluorophenyl)-1-methyl-4,5-dihydro-[1,2,4]triazolo[4,3-f]pteridine (Intermediate EE′),
(4R)-S-(3-(benzyloxy)cyclopentyl)-7-chloro-4-ethyl-4,5-dihydro-[1,2,4]triazolo[4,3-f]pteridine (Intermediate FF-2),
(4R)-S-(3-(benzyloxy)cyclopentyl)-7-chloro-4-ethyl-1-methyl-4,5-dihydro-[1,2,4]triazolo[4,3-f]pteridine (Intermediate FF′-2),
7-chloro-5-isopropyl-5H-spiro[[1,2,4]triazolo[4,3-f]pteridine-4,1′-cyclobutane] (Intermediate GG),
7-chloro-5-isopropyl-1-methyl-5H-spiro[[1,2,4]triazolo[4,3-f]pteridine-4,1′-cyclobutane] (Intermediate GG′),
7-chloro-5-isopropyl-4,4-dimethyl-4,5-dihydro-[1,2,4]triazolo[4,3-f]pteridine (Intermediate HH),
7-chloro-5-isopropyl-1,4,4-trimethyl-4,5-dihydro-[1,2,4]triazolo[4,3-f]pteridine (Intermediate HH′),
tert-butyl 7-chloro-13a-ethyl-13,13a-dihydro-10H-pyrazino[2,1-h][1,2,4]triazolo[4,3-f]pteridine-12(11H)-carboxylate (Intermediate JJ),
tert-butyl 7-chloro-13a-ethyl-3-methyl-13,13a-dihydro-10H-pyrazino[2,1-h][1,2,4]triazolo[4,3-f]pteridine-12(11H)-carboxylate (Intermediate JJ′),
7-chloro-4-ethyl-5-(1-((2-(trimethylsilyl)ethoxy)methyl)-1H-pyrazol-4-yl)-4,5-dihydro-[1,2,4]triazolo[4,3-f]pteridine (Intermediate KK),
7-chloro-4-ethyl-1-methyl-5-(1-((2-(trimethylsilyl)ethoxy)methyl)-1H-pyrazol-4-yl)-4,5-dihydro-[1,2,4]triazolo[4,3-f]pteridine (Intermediate KK′),
7-chloro-10,11,13,13a-tetrahydro-[1,4]oxazino[3,4-h][1,2,4]triazolo[4,3-f]pteridine (Intermediate LL),
7-chloro-3-methyl-10,11,13,13a-tetrahydro-[1,4]oxazino[3,4-h][1,2,4]triazolo[4,3-f]pteridine (Intermediate LL′),
7-chloro-4-ethyl-5-(3-iodophenyl)-4,5-dihydro-[1,2,4]triazolo[4,3-f]pteridine (Intermediate OO-2),
7-chloro-4-ethyl-5-(3-iodophenyl)-1-methyl-4,5-dihydro-[1,2,4]triazolo[4,3-f]pteridine (Intermediate OO′-2),
7-chloro-4-ethyl-5-(4-iodophenyl)-4,5-dihydro-[1,2,4]triazolo[4,3-f]pteridine (Intermediate PP-2),
7-chloro-4-ethyl-5-(4-iodophenyl)-1-methyl-4,5-dihydro-[1,2,4]triazolo[4,3-f]pteridine (Intermediate PP′-2),
7-chloro-4-ethyl-5-(1-((2-(trimethylsilyl)ethoxy)methyl)-1H-pyrazol-3-yl)-4,5-dihydro-[
1,2,4]triazolo[4,3-f]pteridine (Intermediate QQ),
7-chloro-4-ethyl-1-methyl-5-(1-((2-(trimethylsilyl)ethoxy)methyl)-1H-pyrazol-3-yl)-4,5-dihydro-[1,2,4]triazolo[4,3-f]pteridine (Intermediate QQ′),
(4R)-7-chloro-5-(1-cyclopropylethyl)-4-ethyl-4,5-dihydro-[1,2,4]triazolo[4,3-f]pteridine (Intermediate SS),
(4R)-7-chloro-5-(1-cyclopropylethyl)-4-ethyl-1-methyl-4,5-dihydro-[1,2,4]triazolo[4,3-f]pteridine (Intermediate SS′),
7-chloro-5-(3,4-difluorophenyl)-4-ethyl-4,5-dihydro-[1,2,4]triazolo[4,3-f]pteridine (Intermediate TT),
7-chloro-5-(3,4-difluorophenyl)-4-ethyl-1-methyl-4,5-dihydro-[1,2,4]triazolo[4,3-f]pteridine (Intermediate TT′),
7-chloro-5-(3,4-difluorophenyl)-4-ethyl-4,5-dihydro-[1,2,4]triazolo[4,3-f]pteridine (Intermediate UU),
7-chloro-5-(3,4-difluorophenyl)-4-ethyl-1-methyl-4,5-dihydro-[1,2,4]triazolo[4,3-f]pteridine (Intermediate UU′),
(R)-7-chloro-4-ethyl-5-perdeuteroisopropyl-4,5-dihydro-[1,2,4]triazolo[4,3-f]pteridine (Intermediate VV), and
(R)-7-chloro-4-ethyl-5-perdeuteroisopropyl-1-methyl-4,5-dihydro-[1,2,4]triazolo[4,3-f]pteridine (Intermediate VV′),
(R)-7-chloro-4-cyclopropyl-5-isopropyl-4,5-dihydro-[1,2,4]triazolo[4,3-f]pteridine (Intermediate WW),
(R)-7-chloro-4-cyclopropyl-5-isopropyl-1-methyl-4,5-dihydro-[1,2,4]triazolo[4,3-f]pteridine (Intermediate WW′),
(R)-tert-butyl 4-(7-chloro-4-ethyl-[1,2,4]triazolo[4,3-f]pteridin-5(4H)-yl)piperidine-1-carboxylate (Intermediate YY),
(S)-7-chloro-12a-ethyl-10,11,12,12a-tetrahydropyrrolo[2,1-h][1,2,4]triazolo[4,3-f]pteridine (Intermediate XX),
(S)-7-chloro-12a-ethyl-3-methyl-10,11,12,12a-tetrahydropyrrolo[2,1-h][1,2,4]triazolo[4,3-f]pteridine (Intermediate XX′),
(R)-tert-butyl 4-(7-chloro-4-ethyl-1-methyl-[1,2,4]triazolo[4,3-f]pteridin-5(4H)-yl)piperidine-1-carboxylate (Intermediate YY′),
7-chloro-4-ethyl-4-methyl-5-(3,3,3-trifluoropropyl)-4,5-dihydro-[1,2,4]triazolo[4,3-f]pteridine (Intermediate ZZ), and
7-chloro-4-ethyl-1,4-dimethyl-5-(3,3,3-trifluoropropyl)-4,5-dihydro-[1,2,4]triazolo[4,3-f]pteridine (Intermediate ZZ′).

The following table provides the Intermediate name (column 1) and the starting Intermediate (Int. SM, column 2) to give the dihydropteridin-6(5H)-one (column 3, with LCMS data provided) which is then reacted to give the final Intermediate structure as provided in column 4.

| | Int. | dihydropteridin- | |
|---|---|---|---|
| Int. | SM | 6(5H)-one | Intermediate structure |
| E | E-0 | 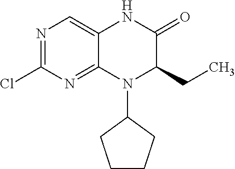 | 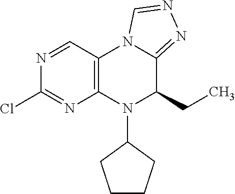 |
| F | | | 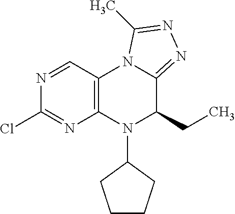 |
| G | G-1 | 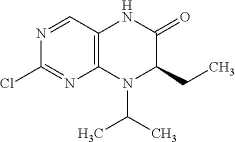 | 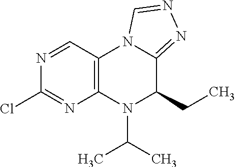 |
| H | | | 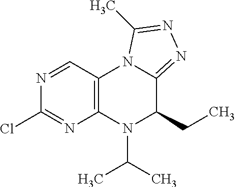 |
| I | I-1 | 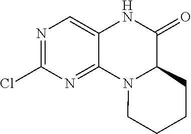 | 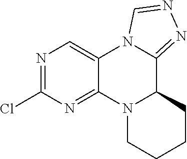 |
| J | | | 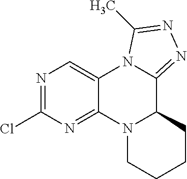 |
| K | K-1 | 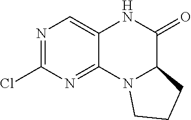 | 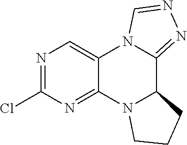 |
| L | | | 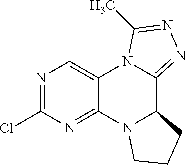 |
| M | M-1 | 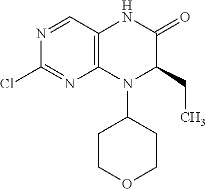 | 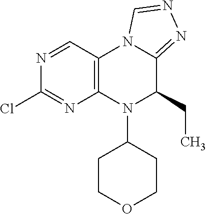 |
| M′ | | | 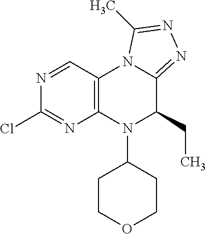 |
| N | N-1 | 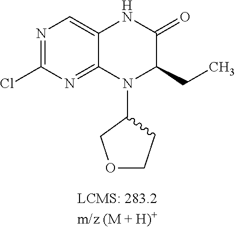 | 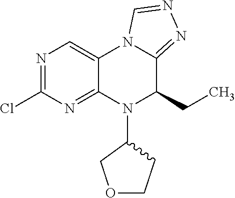 |
| N′ | | | 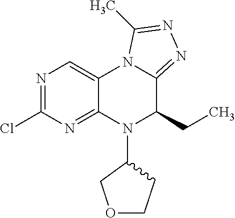 |
| O | O-1 | 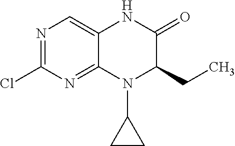 | 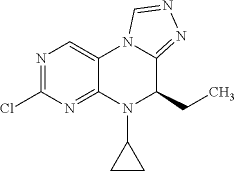 |
| O′ | | | 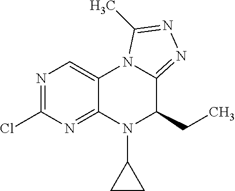 |
| P | P-1 | 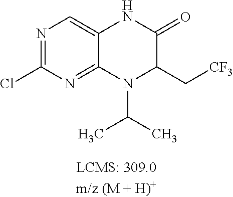 | 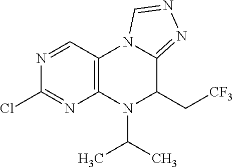 |
| P′ | | | 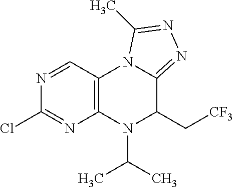 |
| Q | Q-1 | 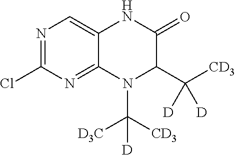 | 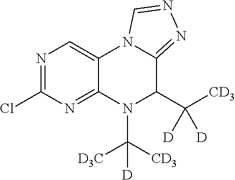 |
| Q′ | | | 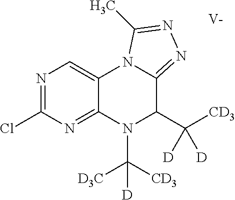 |
| R | R-1 | 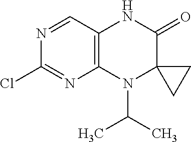 | 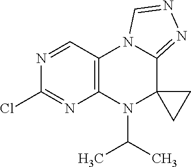 |
| R′ | | | 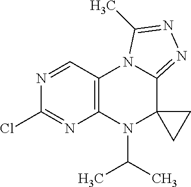 |
| S′ | S-1 | 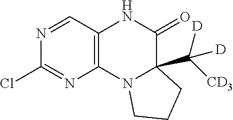 | 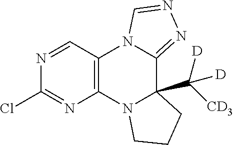 |
| S | | | 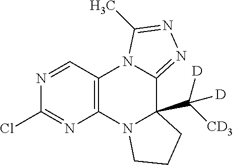 |
| T | T-1 | 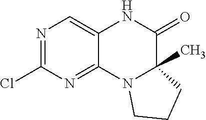 | 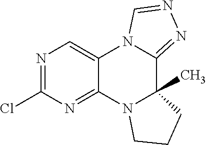 |
| T′ | | | 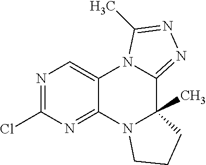 |
| U | U-1 | 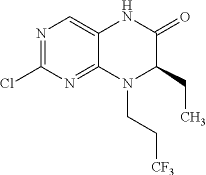 | 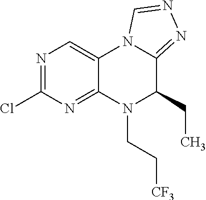 |
| U′ | | | 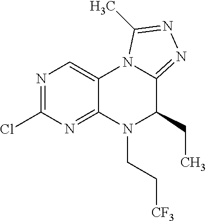 |
| V-2 | V-1 | 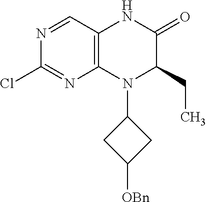 | 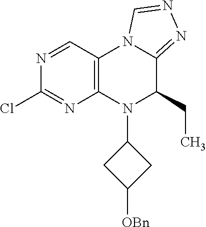 |
| V′-2 | | | 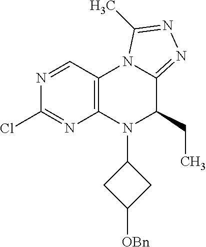 |
| Y | Y-1 | 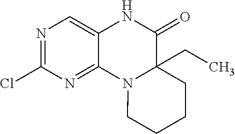 | 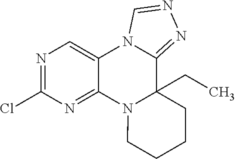 |
| Y′ | | | 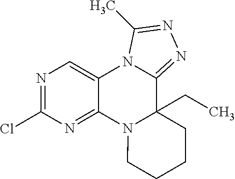 |
| Z | Z-1 | 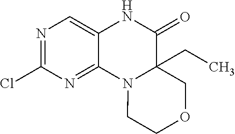 | 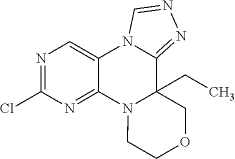 |
| Z′ | | | 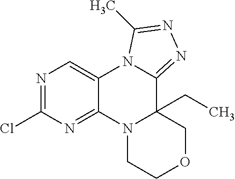 |
| BB | BB-1 | 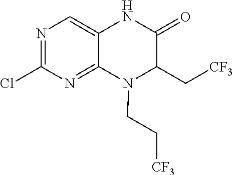 | 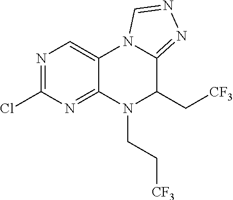 |
| BB′ | | | 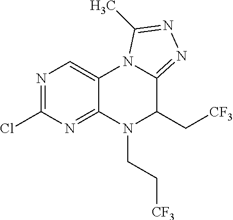 |
| CC | CC-1 | 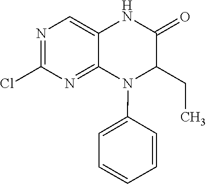 | 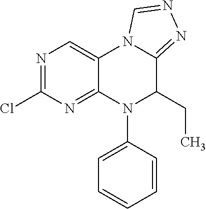 |
| CC′ | | | 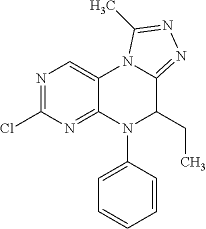 |
| DD | DD-1 | 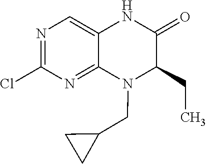 | 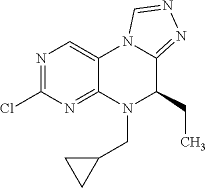 |
| DD′ | | | 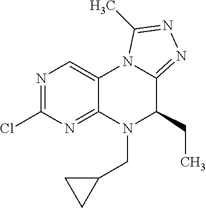 |
| EE | EE-1 | 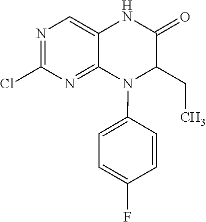 | 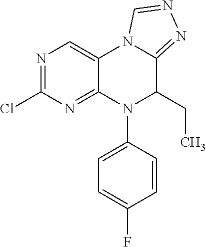 |
| EE′ | | | 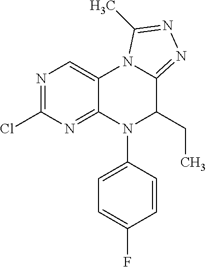 |
| FF-2 | FF-1 | 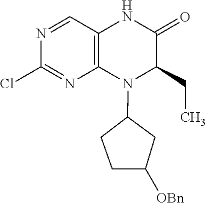 | 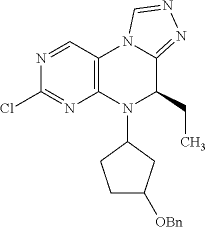 |
| FF′-2 | | | 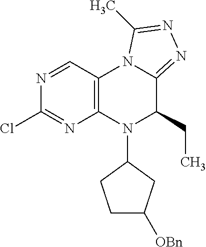 |
| GG | GG-1 | 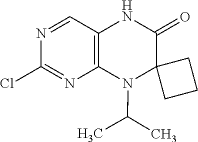 | 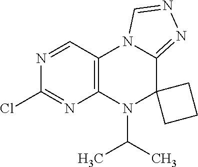 |
| GG′ | | | 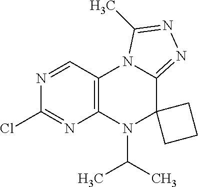 |
| HH | HH-1 | 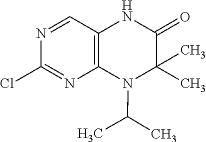 | 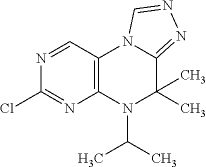 |
| HH′ | | | 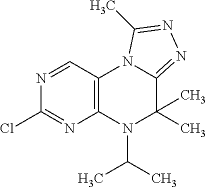 |
| JJ | JJ-1 | 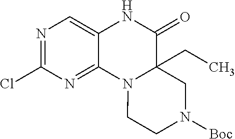 | 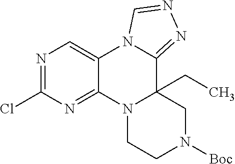 |
| JJ′ | | | 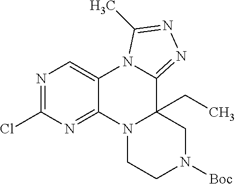 |
| KK | KK-1 | 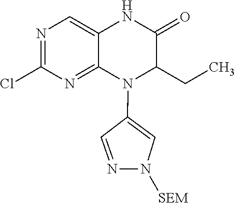 | 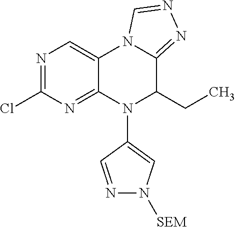 |
| KK′ | | | 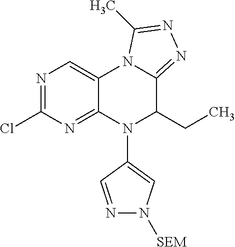 |
| LL | LL-1 | 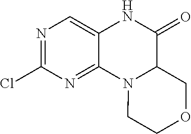 | 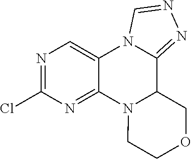 |
| LL′ | | | 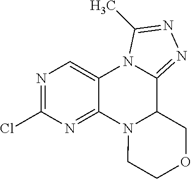 |
| OO-2 | OO-0 | 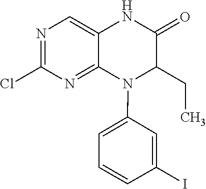 | 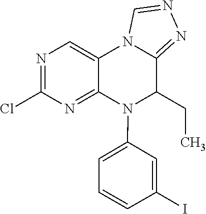 |
| OO′-2 | | | 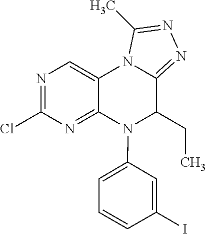 |
| PP-2 | PP-1 | 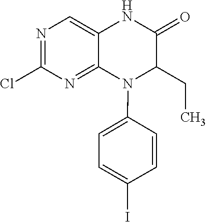 | 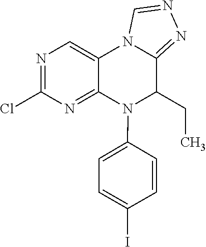 |
| PP′-2 | | | 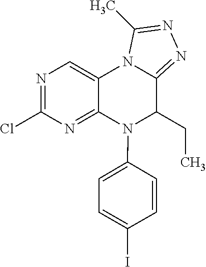 |
| QQ | QQ-1 | 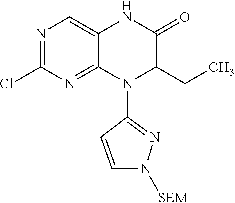 | 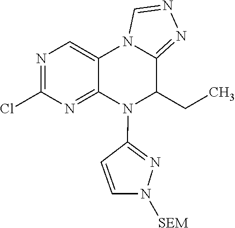 |
| QQ′ | | | 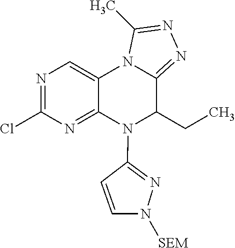 |
| SS | SS-1 | 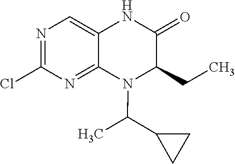 | 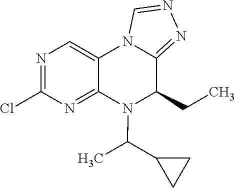 |
| SS′ | | | 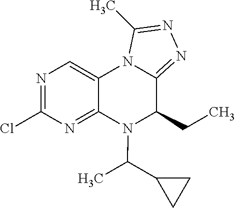 |
| TT | TT-1 | 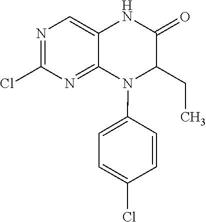 | 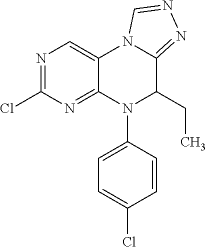 |
| TT′ | | | 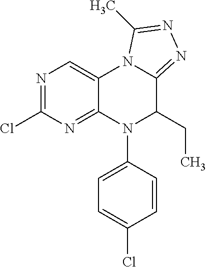 |
| UU | UU-1 | 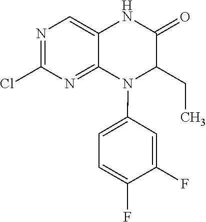 | 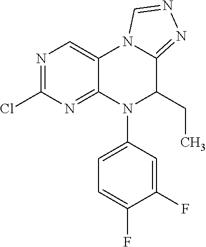 |
| UU′ | | | 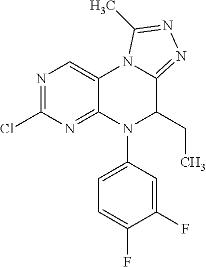 |
| VV | VV-1 | 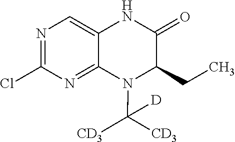 | 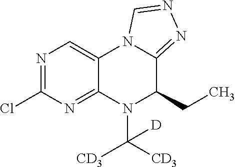 |
| VV′ | | | 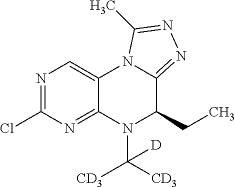 |
| WW | WW-1 | 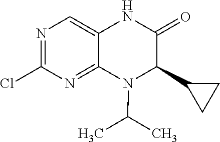 | 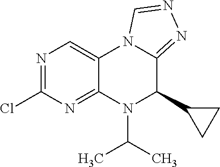 |
| WW′ | | | 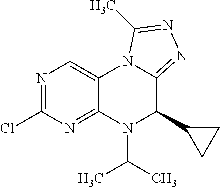 |
| XX | XX-1 | 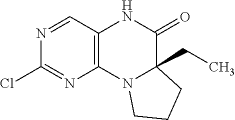 | 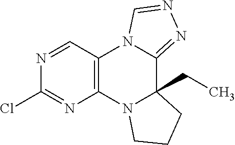 |
| XX′ | | | 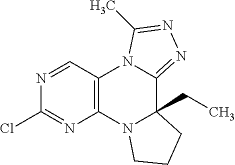 |
| YY | YY-1 | 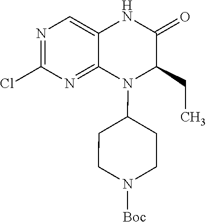 | 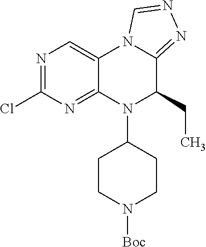 |
| YY′ | | | 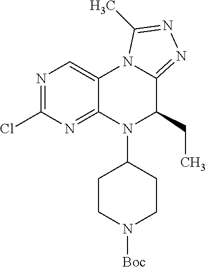 |
| ZZ | ZZ-1 | 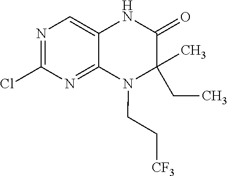 | 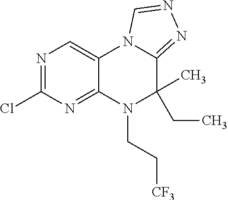 |
| ZZ′ | | | 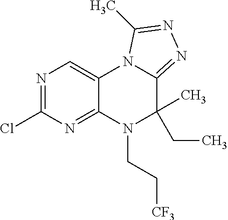 |

### (R)-2-(5-cyclopentyl-4-ethyl-4,5-dihydro-[1,2,4]-triazolo[4,3-f]pteridin-7-yl)-1-phenylethanone (Intermediate E-1) and (R)-2-(5-cyclopentyl-4-ethyl-1-methyl-4,5-dihydro-[1,2,4]-triazolo[4,3-f]pteridin-7-yl)-1-phenylethanone (Intermediate F-1)

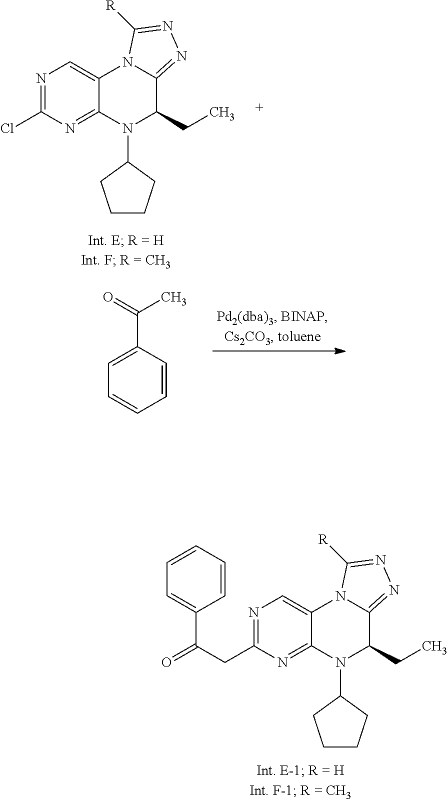

Intermediate E or F, 2.5 eq of acetophenone, 0.05 eq of Pd₂(dba)₃, 0.1 eq of BINAP and 2.0 eq of Cs₂CO₃ are suspended in a mixture of 5:1 toluene and water, then heated to 120° C. under N₂ for 60 hours. After cooling to rt, water is added and the organic phase is washed, dried with anhydrous Na₂SO₄, concentrated and purified by silica gel column to give the pure Intermediate E-1 or F-1.
(R)-2-(4-ethyl-5-isopropyl-4,5-dihydro-[1,2,4]triazolo[4,3-f]pteridin-7-yl)-1-phenylethanone (Intermediate G-2),
(R)-2-(4-ethyl-5-isopropyl-1-methyl-4,5-dihydro-[1,2,4]triazolo[4,3-f]pteridin-7-yl)-1-phenylethanone (Intermediate H-2),
(R)-2-(4-ethyl-5-isopropyl-4,5-dihydro-[1,2,4]triazolo[4,3-f]pteridin-7-yl)-1-(4-(trifluoromethyl)phenyl)ethanone (Intermediate G-3),
(R)-2-(4-ethyl-5-isopropyl-1-methyl-4,5-dihydro-[1,2,4]triazolo[4,3-f]pteridin-7-yl)-1-(4-(trifluoromethyl)phenyl)ethanone (Intermediate H-3),
(R)-2-(4-ethyl-5-isopropyl-4,5-dihydro-[1,2,4]triazolo[4,3-f]pteridin-7-yl)-1-(4-fluorophenyl)ethanone (Intermediate G-4),
(R)-2-(4-ethyl-5-isopropyl-1-methyl-4,5-dihydro-[1,2,4]triazolo[4,3-f]pteridin-7-yl)-1-(4-fluorophenyl)ethanone (Intermediate H-4),
(R)-2-(4-ethyl-5-isopropyl-4,5-dihydro-[1,2,4]triazolo[4,3-f]pteridin-7-yl)-1-(thiazol-2-yl)ethanone (Intermediate G-5),
(R)-2-(4-ethyl-5-isopropyl-1-methyl-4,5-dihydro-[1,2,4]triazolo[4,3-f]pteridin-7-yl)-1-(thiazol-2-yl)ethanone (Intermediate H-5),
3-(4-ethyl-7-(2-(4-fluorophenyl)-2-oxoethyl)-[1,2,4]triazolo[4,3-f]pteridin-5(4H)-yl)benzonitrile (Intermediate OO-1),
3-(4-ethyl-7-(2-(4-fluorophenyl)-2-oxoethyl)-1-methyl-[1,2,4]triazolo[4,3-f]pteridin-5(4H)-yl)benzonitrile (Intermediate OO′-1),
4-(4-ethyl-7-(2-(4-fluorophenyl)-2-oxoethyl)-[1,2,4]triazolo[4,3-f]pteridin-5(4H)-yl)benzonitrile (Intermediate PP-3),
4-(4-ethyl-7-(2-(4-fluorophenyl)-2-oxoethyl)-1-methyl-[1,2,4]triazolo[4,3-f]pteridin-5(4H)-yl)benzonitrile (Intermediate PP′-3),
(S)-2-(12a-ethyl-10,11,12,12a-tetrahydropyrrolo[2,1-h][1,2,4]triazolo[4,3-f]pteridin-7-yl)-1-phenylethanone (Intermediate XX-2),
(S)-2-(12a-ethyl-3-methyl-10,11,12,12a-tetrahydropyrrolo[2,1-h][1,2,4]triazolo[4,3-f]pteridin-7-yl)-1-phenylethanone (Intermediate XX′-2),
(S)-2-(12a-ethyl-10,11,12,12a-tetrahydropyrrolo[2,1-h][1,2,4]triazolo[4,3-f]pteridin-7-yl)-1-(thiazol-2-yl)ethanone (Intermediate XX-3),
(S)-2-(12a-ethyl-3-methyl-10,11,12,12a-tetrahydropyrrolo[2,1-h][1,2,4]triazolo[4,3-f]pteridin-7-yl)-1-(thiazol-2-yl)ethanone (Intermediate XX′-3),
   are prepared similarly to Intermediates E-1 and F-1, where Intermediate G or H is used instead of Intermediate E or F, and with 4-trifluoromethylacetophenone, 4-trifluoromethylacetophenone, and 1-(thiazol-2-yl)ethanone used instead of acetophenone. The following table provides the Intermediate name (column 1), Intermediate used in the reaction (column 2), and ketone (column 3) to give the Intermediate structure given in column 4.

| Inter- | | | |
|---|---|---|---|
| me- | Int. | Ketone | |
| diate | reactant | reactant | Intermediate structure |
| G-2 | G | 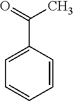 | 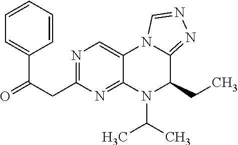 |
| H-2 | H | 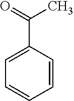 | 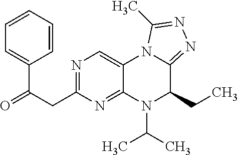 |
| G-3 | G | 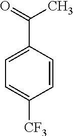 | 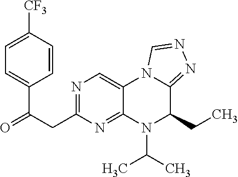 |
| H-3 | H | 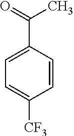 | 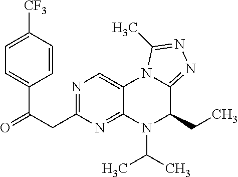 |
| G-4 | G | 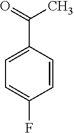 | 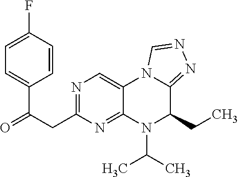 |
| H-4 | H | 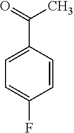 | 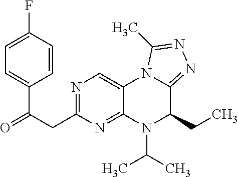 |
| G-5 | G | 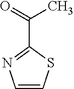 | 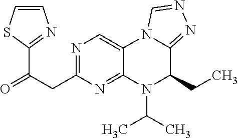 |
| H-5 | H | 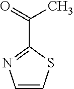 | 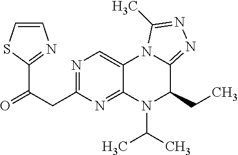 |
| OO-1 | OO | 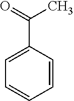 | 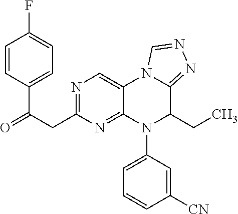 |
| OO′-1 | OO′ | 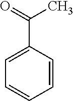 | 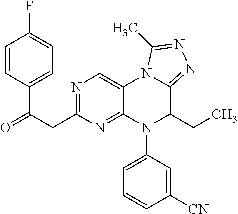 |
| PP-3 | PP | 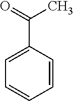 | 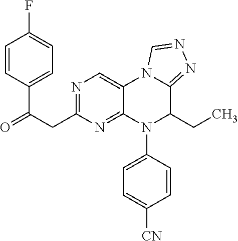 |
| PP′-3 | PP′ | 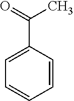 | 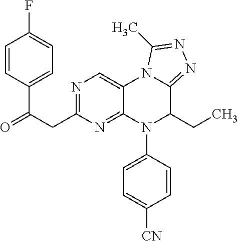 |
| XX-2 | XX | 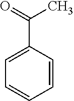 | 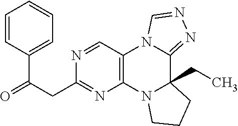 |
| XX′-2 | XX′ | 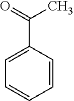 | 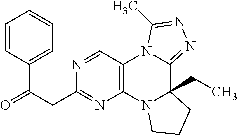 |
| XX-3 | XX | 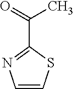 | 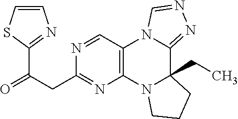 |
| XX′-3 | XX′ | 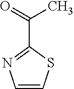 | 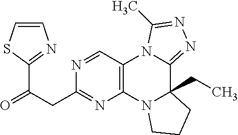 |

### (R)-5-cyclopentyl-4-ethyl-7-hydrazinyl-4,5-dihydro-[1,2,4]-triazolo[4,3-f]pteridine (Intermediate E-2) and (R)-5-cyclopentyl-4-ethyl-7-hydrazinyl-1-methyl-4,5-dihydro-1,2,4]-triazolo[4,3-f]pteridine (Intermediate F-2)

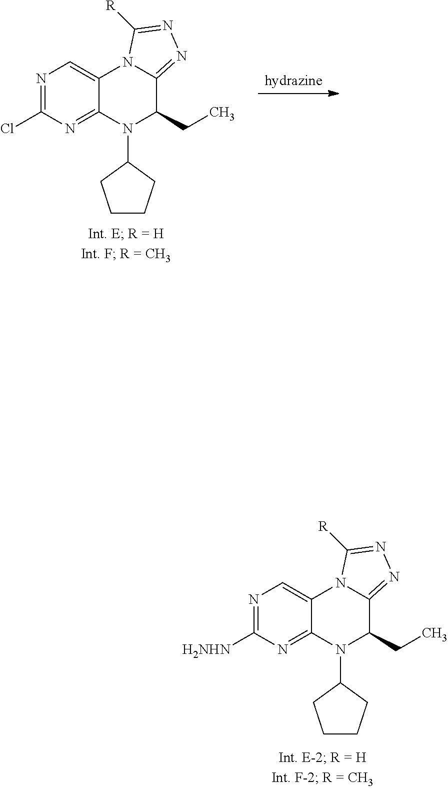

Intermediate E or F and hydrazine (6 equivalents) in ethanol is heated in a microwave for 1 h at 120° C. The solvent is removed to give Intermediate E-2 or F-2.

Intermediate G or Intermediate H is reacted similarly to give (R)-4-ethyl-7-hydrazinyl-5-isopropyl-4,5-dihydro-[1,2,4]triazolo[4,3 4] pteridine (Intermediate G-6) or (R)-4-ethyl-7-hydrazinyl-5-isopropyl-1-methyl-4,5-dihydro-[1,2,4]triazolo[4,3-f]pteridine (Intermediate H-6):

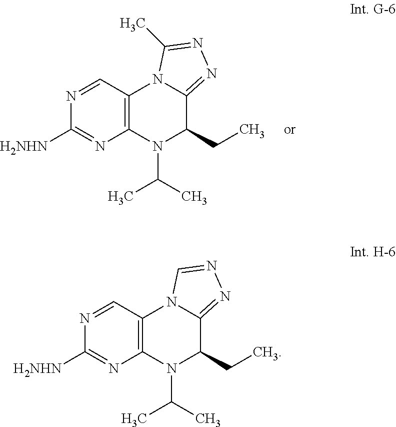

### (R)-2-(4-ethyl-5-isopropyl-4,5-dihydro-[1,2,4]-triazolo[4,3-f]pteridin-7-yl)-1-(1-((2-(trimethylsilyl)ethoxy)methyl)-1H-pyrazol-5-yl)ethanone (Intermediate G-7) and (R)-2-(4-ethyl-5-isopropyl-1-methyl-4,5-dihydro-[1,2,4]-triazolo[4,3-f]pteridin-7-yl)-1-(1-((2-(trimethylsilyl)ethoxy)methyl)-1H-pyrazol-5-yl)ethanone (Intermediate H-7)

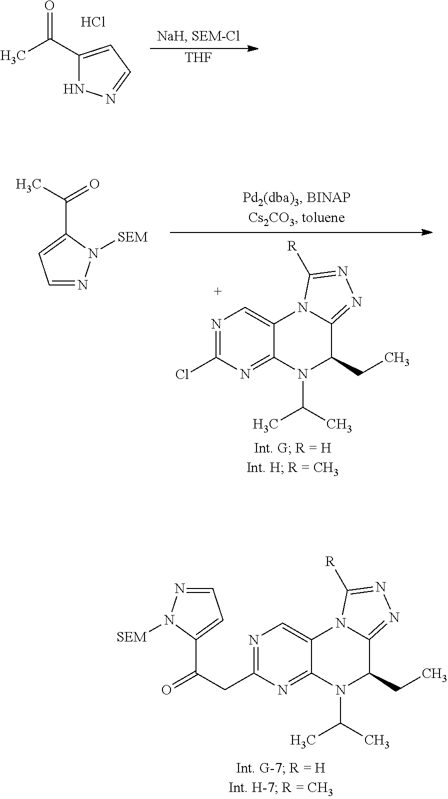

To a suspension of sodium hydride (3.07 g, 76.75 mmol) in 100 mL of anhydrous THF cooled to 0° C. under N₂ (g) inlet was added 1-(1H-pyrazole-5-yl)ethan-1-one hydrochloride (3.09 g, 21.08 mmol). After warming to rt over 1 h, a solution of 2-(trimethylsilyl)ethoxymethyl chloride (4.5 mL, 25.43 mmol) in 100 mL of anhydrous THF was added to the reaction flask via cannulation. The reaction was quenched with water and extracted with EtOAc after 2 h. The organic phase was collected, dried with sodium sulfate, filtered and concentrated under reduced pressure followed by purification by flash chromatography (silica, 50:50 EtOAc/hexane) to give 1-(1-((2-(trimethylsilyl)ethoxy)methyl)-1H-pyrazol-5-yl)ethanone. LCMS; 241.1 m/z (M+H)⁺.

Intermediates G-7 or Intermediate H-7 are prepared similarly to the synthetic methods used to prepare intermediate E-1, with Intermediate G or H instead of Intermediate E or F and with 1-(1-((2-(trimethylsilyl)ethoxy)methyl)-1H-pyrazol-5-yl)ethanone instead of acetophenone.

2-(4-ethyl-5-(1-methyl-1H-pyrazol-4-yl)-4,5-dihydro-[1,2,4]triazolo[4,3-f]pteridin-7-yl)-1-(1-((2-(trimethylsilyl)ethoxy)methyl)-1H-pyrazol-5-yl)ethanone (Intermediate KK-5) and 2-(4-ethyl-1-methyl-5-(1-methyl-1H-pyrazol-4-yl)-4,5-dihydro-[1,2,4]triazolo[4,3-f]pteridin-7-yl)-1-(1-(2-(trimethylsilyl)ethoxy)methyl)-1H-pyrazol-5-yl)ethanone (Intermediate KK′-5)

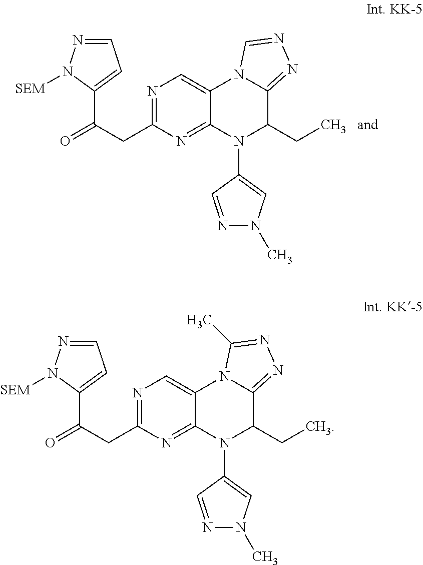

is prepared similarly, with Intermediate KK-3 instead of Intermediate C.

### (R)-7-chloro-5-(3,3-difluorocyclobutyl)-4-ethyl-4,5-dihydro-[1,2,4]-triazolo[4,3-f]pteridine (Intermediate V) and (R)-7-chloro-5-(3,3-difluorocyclobutyl)-4-ethyl-1-methyl-4,5-dihydro-[1,2,4]triazolo[4,3-f]pteridine (Intermediate V′)

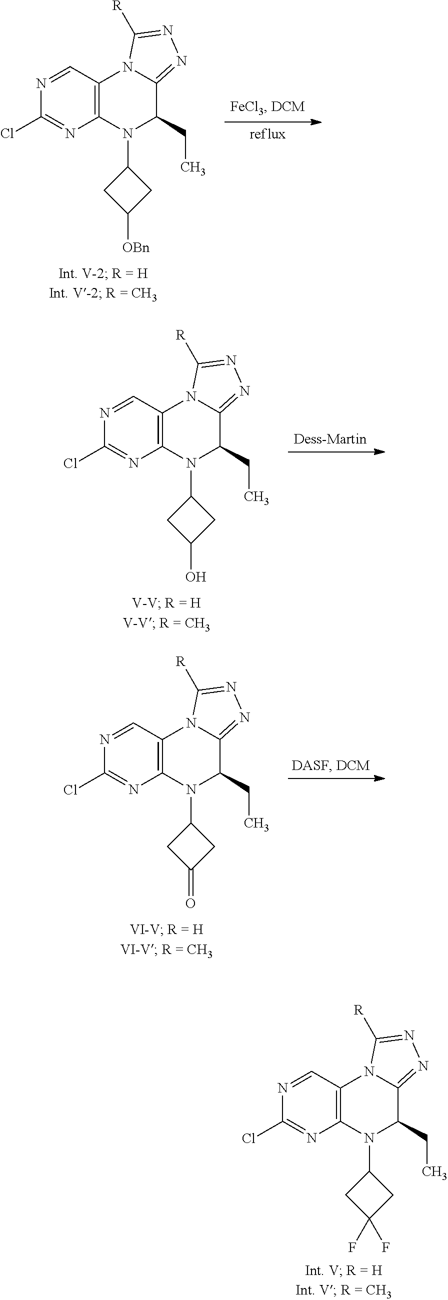

To a stirring mixture of Intermediate V-2 or V′-2 (1 eq) in DCM at rt, FeCl₃ (10 eq) is added. The reaction mixture is heated at reflux for 1 h, then cooled to rt and slowly diluted with DCM and a solution of 3 N NaOH. The resulting mixture is stirred at rt for 30 min before the layers are separated. The aqueous layer is extracted 2× with DCM. The organic layers are dried over MgSO₄, filtered, and concentrated under reduced pressure. The crude compound V-V or V-V′ is further purified by MPLC.

To a stirring mixture of compound V-V or V-V′ (1 eq) in DCM at rt, NaHCO₃ (6.0 eq) and Dess-Martin reagent (4.55 eq) are added. The reaction mixture is stirred at rt until all the alcohol is consumed. The reaction mixture is slowly quenched with a saturated NaHCO₃ and Na₂S₂O₃ solution (1:1 in volume). A normal aqueous work up with DCM is followed. The crude product is further purified by MPLC to give the ketone compound VI-V or VI-V′.

To a stirring mixture of compound VI-V or VI-V′ (1 eq) in DCM at 0° C., DAST (5.0 eq) is added. The reaction mixture is slowly warmed up to rt overnight. The resulting mixture is poured over an ice cold water beaker. The mixture is allowed to stir at rt for 10 min. A normal work up with DCM is followed. The crude product is purified by MPLC to give Intermediate V or V′.

(R)-7-chloro-4-ethyl-5-(3-fluorocyclobutyl)-4,5-dihydro-[1,2,4]triazolo[4,3-f]pteridine (Intermediate W) and (R)-7-chloro-4-ethyl-5-(3-fluorocyclobutyl)-1-methyl-4,5-dihydro-[1,2,4]triazolo[4,3-f]pteridine (Intermediate W′)

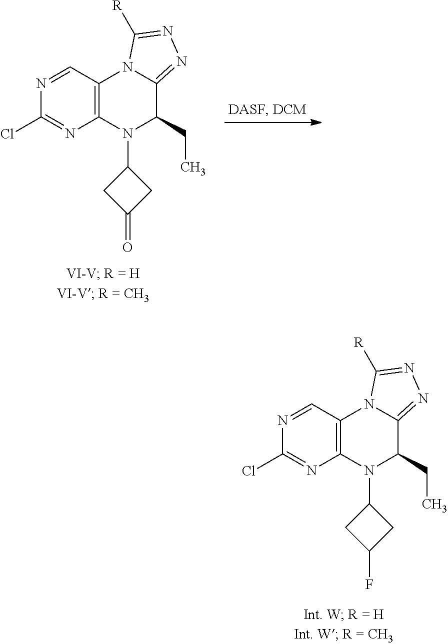

are prepared similarly from compound VI-V or VI-V′, where DAST is used at 4.0 equivalents instead of 5.0 equivalents.

(4R)-7-chloro-5-(3,3-difluorocyclopentyl)-4-ethyl-4,5-dihydro-[1,2,4]triazolo[4,3-f]pteridine (Intermediate FF) and (4R)-7-chloro-5-(3,3-difluorocyclopentyl)-4-ethyl-1-methyl-4,5-dihydro-[1,2,4]triazolo[4,3-f]pteridine (Intermediate FF′)

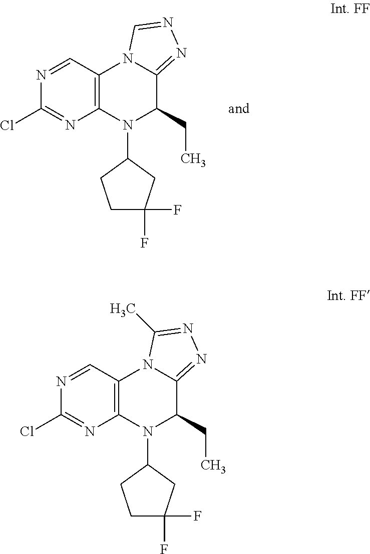

are prepared similarly with Intermediate FF-2 or FF′-2 instead of Intermediate V-2 or V′-2.

### (+/−)-7-chloro-14a-ethyl-10,11,12,13,14,14a-hexahydroazepino[2,1-h][1,2,4]-triazolo[4,3-f]pteridine (Intermediate X) and 7-chloro-14a-ethyl-3-methyl-10,11,12,13,14,14a-hexahydroazepino[2,1-h][1,2,4]-triazolo[4,3-f]pteridine (Intermediate X′)

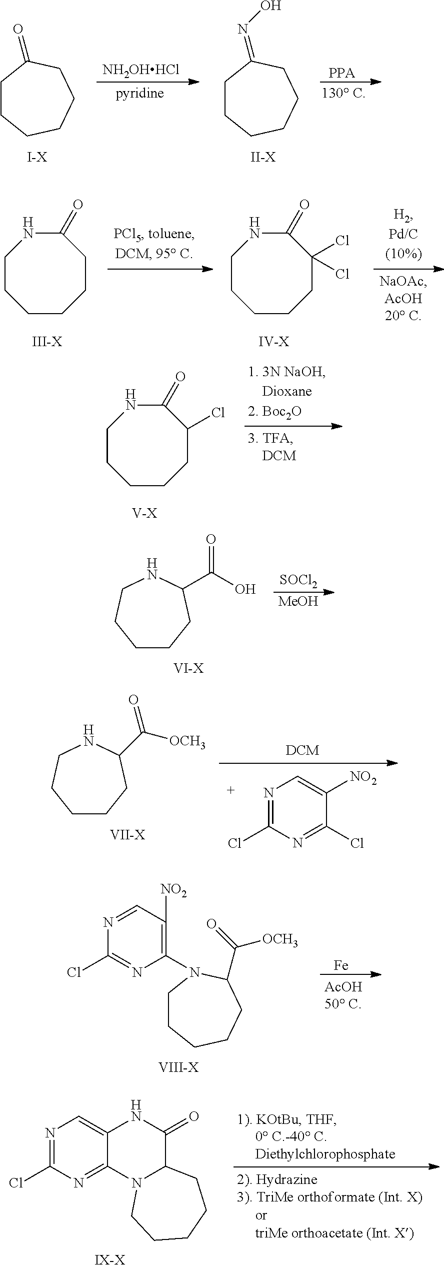

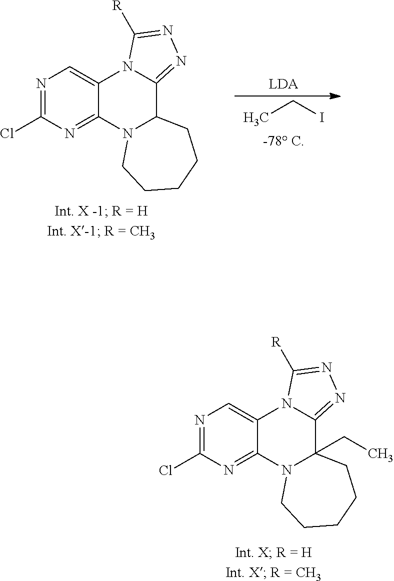

To a solution of cycloheptanone (I-X, 1.0 eq) and pyridine (1.5 eq), NH₂OH, HCl salt (1.1 eq) was added at 0° C. After stirring for 10 min at 0° C., the mixture was allowed to warm to rt and stirred 18 h, then solvent was evaporated. The residue was washed with EtOAc, and the filtrate was evaporated to give compound II-X.

Water (6.0 eq) was added to PPA (P₂O₅ 80%, 2.6 eq), then heated to 130° C.; and compound II-X (1.0 eq) was added at such a rate that the temperature was maintained between 130-140° C. The solution was kept at 130° C. for 1 h and slowly cooled to 100° C. The mixture was then stirred with ice water, then extracted with DCM. The organic layer was dried with Na₂SO₄ and concentrated to give compound III-X.

Compound III-X (1.0 eq) in DCM was slowly added to a stirred suspension of PCl₅ (2.0 eq) in toluene. After heating under reflux for 2 h, the brown solution was concentrated. Ice was added to the residue followed by acetone, then aqueous 10% NaHCO₃ solution was added until pH=8. After stirring 16 h, the solution was extracted with DCM, and the extract was dried over Na₂SO₄, filtered, and concentrated under reduced pressure to give an orange oil, which was purified by silica column chromatography (EA/PE=1:5-1:3) to give compound IV-X.

Compound IV-X (1.0 eq) was dissolved in AcOH, 10% Pd/C (0.1 eq) and NaOAc (2.8 eq) were added and the mixture was hydrogenated at 20° C. for 18 h. The catalyst was removed by filtration and the filtrate was evaporated. The residue was neutralized with 10% Na₂CO₃ solution and extracted with DCM several times. The extract was concentrated, and the residue was crystallized from DCM/PE to give compound V-X.

A suspension of compound V-X (1.0 eq) in 3N NaOH (9.0 eq) and dioxane was refluxed for 18 h, then the solution was cooled to rt and Boc₂O (2.0 eq) was added to the mixture followed by dioxane. The reaction mixture was stirred for 4 h, then the mixture was washed with DCM to remove diketopiperazine by-product. The resulting aqueous phase was acidified with concentrated HCl and extracted with DCM. The extract was evaporated to give a colorless oil. The oil was dissolved in DCM, TFA was added and stirred at rt for 30 min. The mixture was evaporated to give an oil, which was washed with DCM/Et₂O to give compound VI-X.

To compound VI-X (1.0 eq) in methanol, SOCl₂ (2.5 eq) was added drop-wise at 0° C. The mixture was stirred at rt for 16 h, then was evaporated and the residue was diluted with DCM and washed with saturated Na₂CO₃ solution. The organic phase was then evaporated to give compound VII-X.

Compound VII-X (1.0 eq) and 2,4-dichloro-5-nitropyrimidine (1.0 eq) were dissolved in DCM, then K₂CO₃ (1.5 eq) was added. The resulting suspension was stirred at rt for 16 h. The mixture was diluted with DCM, then washed with water and brine. The combined organic phases were dried over Na₂SO₄, evaporated and purified by silica column (EtOAc/PE=1:7) to give compound VIII-X.

To compound VIII-X (1.0 eq) in AcOH, Fe (10.0 eq) was added and stirred at 50° C. for 1.5 h. The mixture was filtered and the filtrate was evaporated, the residue was dissolved in DCM, and then washed with saturated NaHCO₃. The aqueous phase was extracted with DCM. The combined organic phase was dried over Na₂SO₄, evaporated and purified by silica column chromatography (EtOAc/PE=1:3 to 1:1) to give compound IX-X.

7-Chloro-10,11,12,13,14,14a-hexahydroazepino[2,1-h][1,2,4]triazolo[4,3-f]pteridine (Intermediate X-1) and 7-chloro-3-methyl-10,11,12,13,14,14a-hexahydroazepino[2,1-h][1,2,4]triazolo[4,3-f]pteridine (Intermediate X′-1) are prepared from compound IX-X similarly to the conversion of Intermediate B to Intermediate C and Intermediate D.

n-BuLi (2.5 M solution in hexane, 1.5 eq) is added dropwise to a stirred solution of diisopropylamine (1.6 eq) in dry THF at −78° C. under Ar. The solution is stirred for 5 min at −78° C., then warmed to 0° C. and stirred for another 20 min. The resulting solution is added dropwise to a solution of Intermediate X-1 or Intermediate X′-1 (1.0 eq) in dry THF at −78° C.; this is stirred for a further 40 min then MeI (3.0 eq) is added and the solution is stirred for 40 min at −78° C. Water is added, the solution is warmed to rt and extracted 3× with EtOAc. The combined organic phases are dried with solid Na₂SO₄, evaporated and purified by silica column chromatography (EtOAc: PE=1:2) to give the title compounds.

### 2-(13a-ethyl-10,11,13,13a-tetrahydro-[1,4]oxazino[3,4-h][1,2,4]-triazolo[4,3-f]pteridin-7-yl)-1-phenylethanone (Intermediate Z-2) and 2-(13a-ethyl-3-methyl-10,11,13,13a-tetrahydro-[1,4]oxazino[3,4-h][1,2,4]-triazolo[4,3-f]pteridin-7-yl)-1-phenylethanone (Intermediate Z′-2)

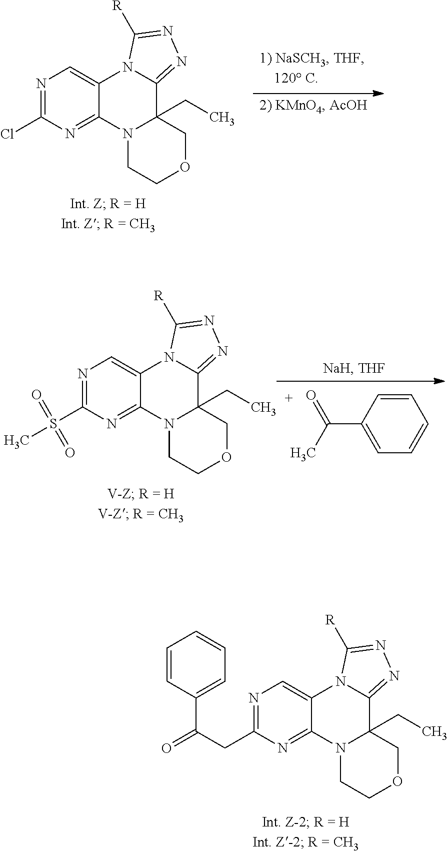

Intermediate Z-1 or Z′-1 (0.707 mmol), sodium methanethiolate (2.12 mmol) and THF are combined in a sealed tube and heated to 120° C. for 18 h. The reaction mixture is cooled to rt, diluted with EtOAc, washed with water, dried with Na₂SO₄, filtered and concentrated.

The resulting residue is dissolved in AcOH, the temperature is decreased to 0° C., and a solution of KMnO₄ (0.848 mmol) in water is added. The reaction mixture is stirred for 2 h at 0° C., then is quenched with saturated Na₂SO₃ and warmed to rt and extracted 3× into EtOAc. The combined organic layers are dried with Na₂SO₄, filtered and concentrated. The resulting residue is purified by flash chromatography (50% EtOAc in hexanes) to give compound V-Z or V-Z′.

Compound V-Z or V-Z′ is added to a suspension of NaH (1.81 mmol) and acetophenone (1.64 mmol) in THF with stirring at 0° C. The reaction mixture is stirred for 18 h while slowly warming to rt. The reaction mixture is quenched with saturated NH₄Cl, diluted with EtOAc, and the two layers were separated. The organic layer is dried with Na₂SO₄, filtered and concentrated. The resulting residue is purified by flash chromatography (50% EtOAc in hexanes) to provide the title compounds.
2-(13a-ethyl-10,11,13,13a-tetrahydro-[1,4]oxazino[3,4-h][1,2,4]triazolo[4,3-f]pteridin-7-yl)-1-(thiazol-2-yl)ethanone (Intermediate Z-3),
2-(13a-ethyl-3-methyl-10,11,13,13a-tetrahydro-[1,4]oxazino[3,4-h][1,2,4]triazolo[4,3-f]pteridin-7-yl)-1-(thiazol-2-yl)ethanone (Intermediate Z′-3)
1-(2,4-difluorophenyl)-2-(13a-ethyl-10,11,13,13a-tetrahydro-[1,4]oxazin[3,4-h][1,2,4]triazolo[4,3-f]pteridin-7-yl)ethanone (Intermediate Z-4),
1-(2,4-difluorophenyl)-2-(13a-ethyl-3-methyl-10,11,13,13a-tetrahydro-[1,4]oxazino[3,4-h][1,2,4]triazolo[4,3-f]pteridin-7-yl)ethanone (Intermediate Z′-4),
2-(13a-ethyl-10,11,13,13a-tetrahydro-[1,4]oxazino[3,4-h][1,2,4]triazolo[4,3-f]pteridin-7-yl)-1-(5-fluoropyridin-2-yl)ethanone (Intermediate Z-5), and 2-(13a-ethyl-3-methyl-10,11,13,13a-tetrahydro-[1,4]oxazino[3,4-h][1,2,4]triazolo[4,3-f]pteridin-7-yl)-1-(5-fluoropyridin-2-yl)ethanone (Intermediate Z′-5),

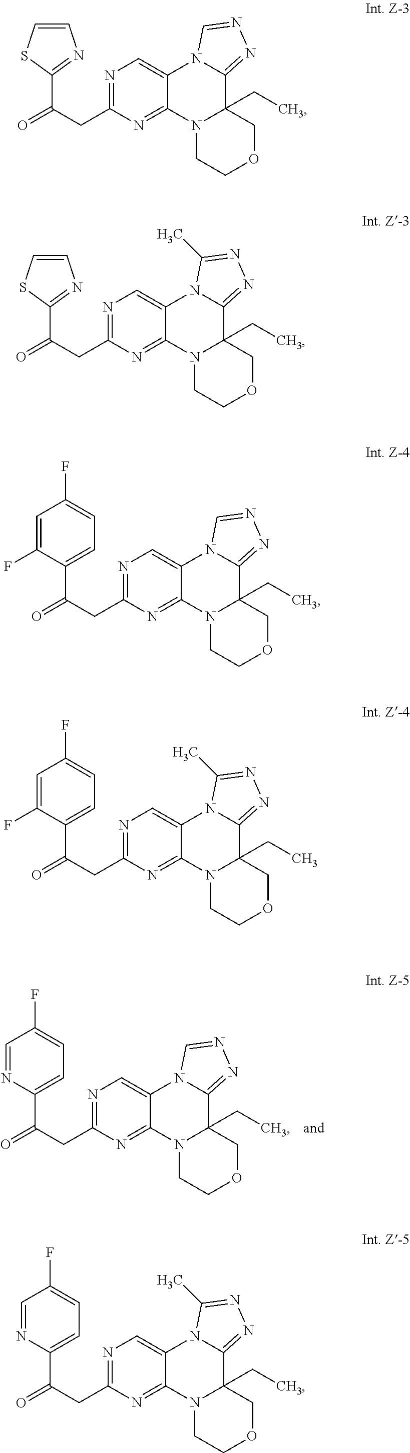

Intermediate Z-3, Z′-3. Z-4, Z′-4, Z-5, and Z′-5 are prepared from Intermediate Z or Z′ similarly to the method used for Intermediate Z-2 or Z′-2, with 1-(thiazol-2-yl)ethanone, 2,4-difluorophenylmethylketone, and 1-(5-fluoropyridin-2-yl)ethanone, respectively, instead of acetophenone.

### 7-chloro-4-ethyl-5-(1-methyl-1H-pyrazol-4-yl)-4,5-dihydro-[1,2,4]-triazolo[4,3-f]pteridine (KK-3) and 7-chloro-4-ethyl-1-methyl-5-(1-methyl-1H-pyrazol-4-yl)-4,5-dihydro-1,2,4]-triazolo[4,3-f]pteridine (Intermediate KK′-3)

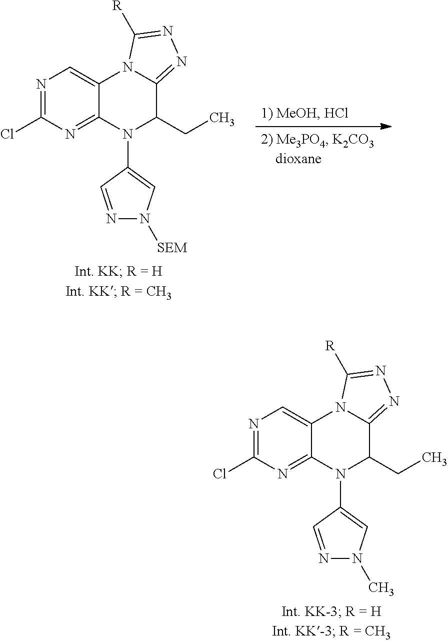

To a stirring mixture of Intermediate KK or KK′ (0.71 mmol) in MeOH, HCl (4N in dioxane) is added. The resulting mixture is warmed to reflux until all the starting material is consumed. The reaction mixture is cooled to rt and concentrated. The crude residue is diluted with EtOAc and neutralized with a saturated NaHCO₃ solution. The layers were separated and the aqueous layer is extracted 2× with EtOAc. The organic layers were dried over MgSO₄, filtered, and concentrated under reduced pressure. The crude material is purified by MPLC to give the compound with nitrogen protecting group removed. This is dissolved in dioxane and K₂CO₃ and Me₃PO₄ are added. The resulting mixture is stirred at 100° C. overnight. The reaction mixture is cooled to rt and diluted with water and EtOAc. The layers are separated and the aqueous layer is extracted 2× with EtOAc. The organic layers are dried over MgSO₄, filtered, and concentrated. The crude material is purified by MPLC to give the desired Intermediate KK-3 and KK′-3.

7-chloro-4-ethyl-5-(1-methyl-1H-pyrazol-3-yl)-4,5-dihydro-[1,2,4]triazolo[4,3-f]pteridine (QQ-2) and 7-chloro-4-ethyl-1-methyl-5-(1-methyl-1H-pyrazol-3-yl)-4,5-dihydro-[1,2,4]triazolo[4,3-f]pteridine (Intermediate QQ′-2)

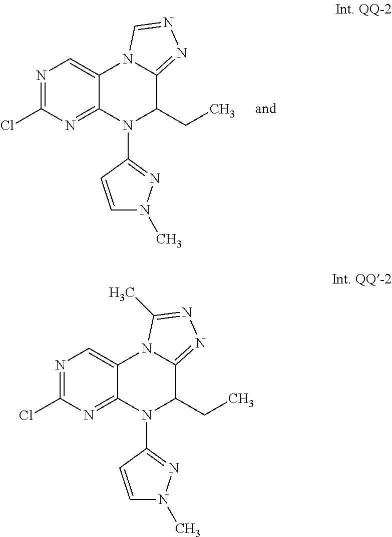

are prepared similarly from Intermediate QQ or QQ′.

(R)-7-chloro-4-ethyl-5-(1-methyl-1H-pyrazol-4-yl)-4,5-dihydro-[1,2,4]triazolo[4,3-f]pteridine (Intermediate KK-2) and (R)-7-chloro-4-ethyl-1-methyl-5-(1-methyl-1H-pyrazol-4-yl)-4,5-dihydro-[1,2,4]triazolo[4,3-f]pteridine (Intermediate KK′-2)

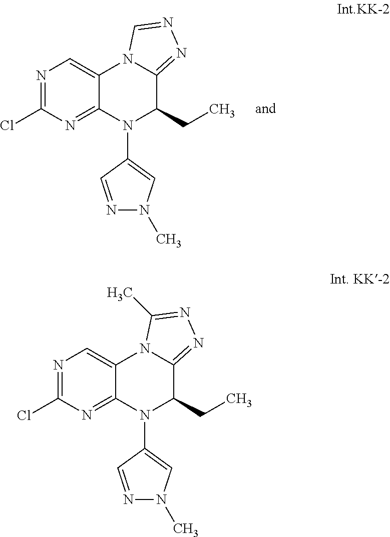

are prepared similarly, where Intermediate KK-1 is separated by chiral chromatography, and the appropriate isomer is carried through to the analog of Intermediate KK or KK′ and reacted similarly to the method above for Intermediate KK-3 or KK′-3 to give Intermediate KK-2 or KK′-2.

### 7-chloro-5-(1-(cyclopropylmethyl)-1H-pyrazol-4-yl)-4-ethyl-4,5-dihydro-[1,2,4]-triazolo[4,3-f]pteridine (Intermediate KK-4) and 7-chloro-5-(1-(cyclopropylmethyl)-1H-pyrazol-4-yl)-4-ethyl-1-methyl-4,5-dihydro-[1,2,4]-triazolo[4,3-f]pteridine (Intermediate KK′-4)

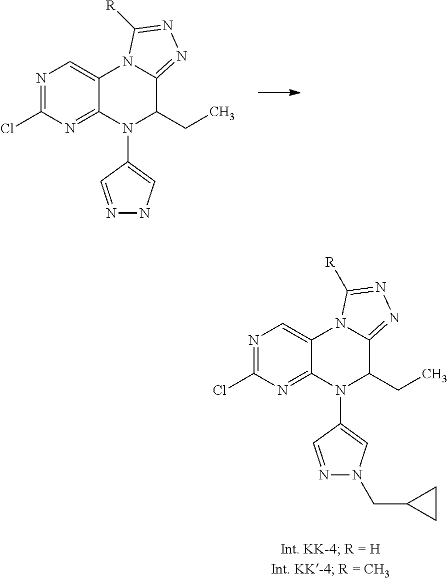

To the Intermediate KK or KK′ with deprotected nitrogen (as prepared in the method of making Intermediate KK-3 or KK′-3, 0.59 mmol) in acetonitrile, cyclopropyl methyl bromide (1.78 mmol), K₁ and K₂CO₃ (1.81 mmol) are added. The reaction mixture is stirred at 90° C. overnight. The resulting mixture is cooled to rt and slowly quenched with a saturated NaHCO₃ solution. The reaction mixture is diluted with EtOAc. The layers are separated and the aqueous layer is extracted 2× with EtOAc. The layers are dried over MgSO₄, filtered, and concentrated, and the resulting material is purified by MPLC to give Intermediate KK-4 or KK′-4.

### 7-chloro-4-ethyl-5-(3-(pyrimidin-5-yl)phenyl)-4,5-dihydro-[1,2,4]-triazolo[4,3-f]pteridine (Intermediate MM) and 7-chloro-4-ethyl-1-methyl-5-(3-(pyrimidin-5-yl)phenyl)-4,5-dihydro-[1,2,4]-triazolo[4,3-f]pteridine (Intermediate MM′)

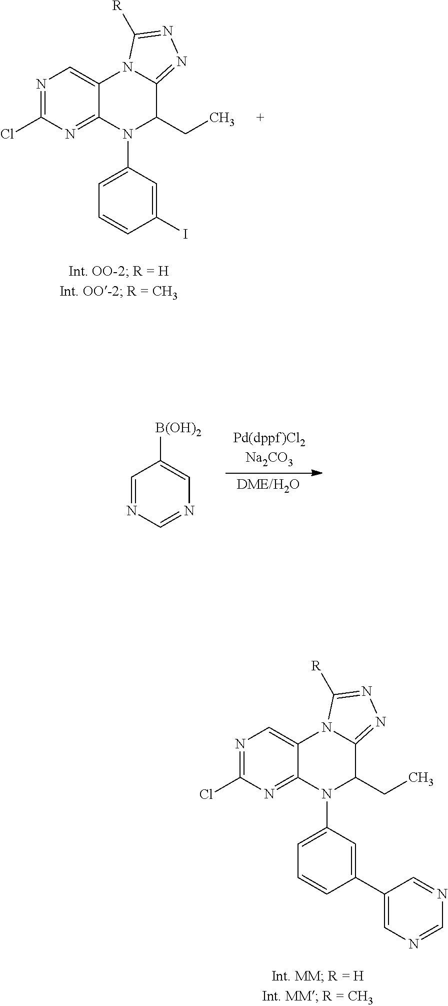

Intermediate OO-2 or OO′-2 (0.116 mmol), pyrimidin-5-ylboronic acid (0.174 mmol), sodium carbonate (0.232 mmol) and Pd(dppf)Cl₂ (0.0116 mmol) are dissolved in DME/H₂O (4/1, v/v) and a stream of nitrogen is bubbled through the mixture for 5 minutes. The resulting solution is stirred at 70° C. for 2 h. The reaction mixture is diluted with brine, extracted with EtOAc, dried with Na₂SO₄ and concentrated to give the title compounds.

### 5-(3-(1H-pyrazol-1-yl)phenyl)-7-chloro-4-ethyl-4,5-dihydro-[1,2,4]-triazolo[4,3-f]pteridine (Intermediate NN) and 5-(3-(1H-pyrazol-1-yl)phenyl)-7-chloro-4-ethyl-1-methyl-4,5-dihydro-[1,2,4]-triazolo[4,3-f]pteridine (Intermediate NN′)

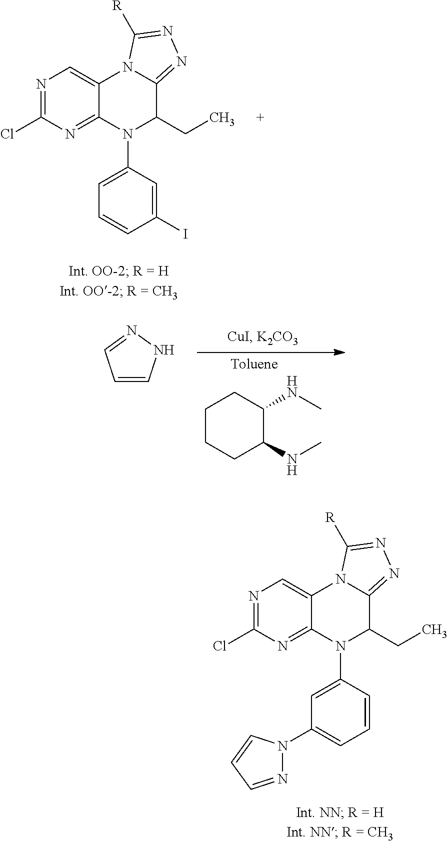

Intermediate OO-2 or OO′-2 (0.116 mmol), pyrazole (0.174 mmol), CuI (0.0116 mmol), trans-1,2-bis(methylamino)cyclohexane (0.0232) and K₂CO₃ (0.232 mmol) are dissolved in toluene in a screw cap vial and a stream of nitrogen is bubbled through the mixture for 2 minutes. The resulting solution is stirred at 80° C. for 8 h. The reaction mixture is diluted with brine, extracted with EtOAc, dried with Na₂SO₄ and purified by silica column (hexane:EtOAc) to give the title compounds.

### 3-(7-chloro-4-ethyl-[1,2,4]triazolo[4,3-f]pteridin-5(4H)-yl)benzonitrile (Intermediate OO) and 3-(7-chloro-4-ethyl-1-methyl-[1,2,4]-triazolo[4,3-f]pteridin-5(4H)-yl)benzonitrile (Intermediate OO′)

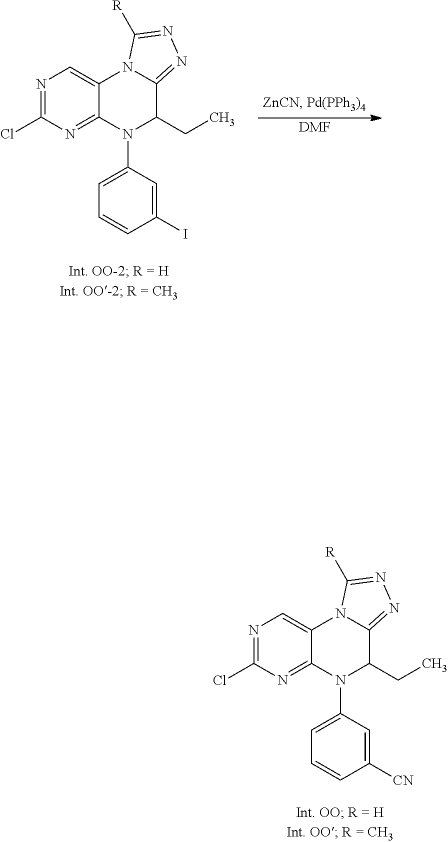

Intermediate OO-2 or OO′-2 (0.256 mmol), zinc cyanide (0.282 mmol) and Pd(PPh₃)₄ (0.0256 mmol) are dissolved in DMF in a screw cap vial and a stream of nitrogen is bubbled through the solution for 5 minutes. The vial is sealed and the reaction mixture is stirred at 100° C. for 18 h. The reaction mixture is purified by silica column (hexane: EtOAc) to give the title compounds.

4-(7-chloro-4-ethyl-[1,2,4]triazolo[4,3-f]pteridin-5(4H)-yl)benzonitrile (Intermediate PP) and 4-(7-chloro-4-ethyl-1-methyl-[1,2,4]triazolo[4,3-f]pteridin-5(4H)-yl)benzonitrile (Intermediate PP′)

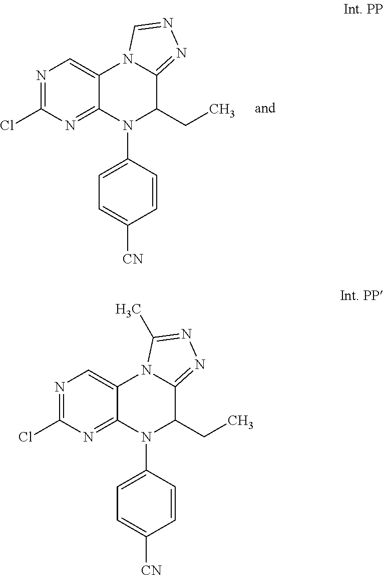

are prepared similarly with Intermediate PP-2 and Intermediate PP′-2 instead of Intermediate OO-2 and Intermediate OO′-2.

### Synthesis of Imidazole Intermediates

A number of methods exist in the literature that describe the synthesis of the required imidazole analogs used in the examples herein. For methods that access imidazoles from aldehydes via the dihydroimidazoles followed by oxidation to the imidazole see: Fujioka et al., Tetrahedron Letters 46 (2005) 2197-2199; Gogoi, Konwar, Tetrahedron Letters 47 (2006) 79-82; Nicolaou et al, J. Am. Chem. Soc. 2004, 126, 5192-5201; or Ishihara, Togo, Synlett. 2006, 227-230. For a one-pot method from aryl and heteroaryl nitriles see: Voss et al. Tetrahedron 2008, 64, 645-51. These references are hereby incorporated by reference herein as they relate to the synthesis of such imidazoles.

### Synthesis of 2-(4-(methylsulfonyl)phenyl)-1H-imidazole (Imidazole 1)

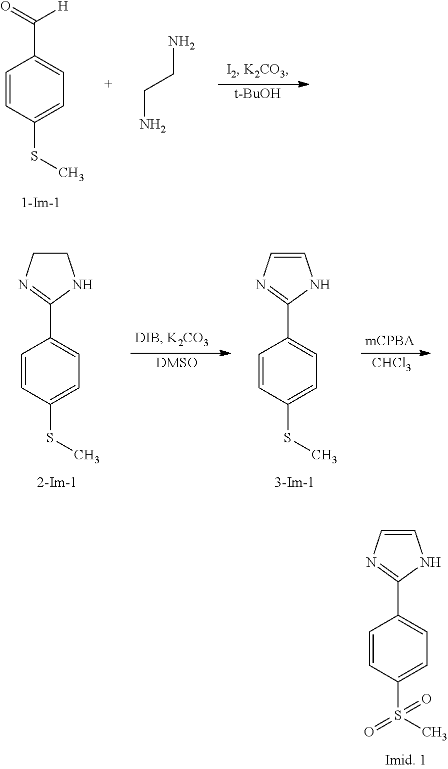

To a solution of 4-(methylthio)benzaldehyde (1-Im-1, 10 g, 1.0 eq) in 1000 mL of t-BuOH, ethylene diamine (1.1 eq) was added. The mixture was stirred at rt under Ar for 30 min, then K₂CO₃ (3.0 eq) and I₂ (1.25 eq) were added to the mixture. This mixture was stirred at 70° C. for 3 h, then was quenched with aqueous Na₂SO₃ until the color of iodine disappeared, then extracted with CHCl₃. The organic layer was washed with NaHCO₃ and brine and dried with Na₂SO₄. The solvent was removed to give 2-(4-(methylthio)phenyl)-4,5-dihydro-1H-imidazole (compound 2-Im-1).

To a solution of 2-(4-(methylthio)phenyl)-4,5-dihydro-1H-imidazole (2-Im-1, 9.6 g, 1.0 eq) in 100 mL of DMSO, DIB (1.1 eq) and K₂CO₃ (1.1 eq) were added. The mixture was heated to 70° C. overnight, then extracted with EtOAc and the organic layer was concentrated to provide 2-(4-(methylthio)phenyl)-1H-imidazole (compound 3-Im-1).

To a stirred solution of 2-(4-(methylthio)phenyl)-1H-imidazole (3-Im-1, 5 g, 1.0 eq) in 50 mL of CHCl₃, m-CPBA (2.0 eq) was added and the reaction was stirred at rt for 1 h, then washed with 5% aqueous Na₂SO₃ and aqueous Na₂CO₃ and extracted with EtOAc. The organic layer was dried over Na₂SO₄, concentrated and the residue was purified by silica column (80% EtOAc: 20% MeOH) to give Imidazole 1. LCMS (0.01% Ammonia): 223.1 m/z (M+H)⁺; ¹H-NMR (DMSO-d6, 500 MHz): δ: 12.87 (s, 1H), 8.17 (d, 2H, J=8.5 Hz), 8.00 (d, 2H, J=8.5 Hz), 7.38 (s, 1H), 7.13 (s, 1H), 3.25 (s, 3H).

### Synthesis of 2-(1H-imidazol-2-yl)thiazole (Imidazole 2)

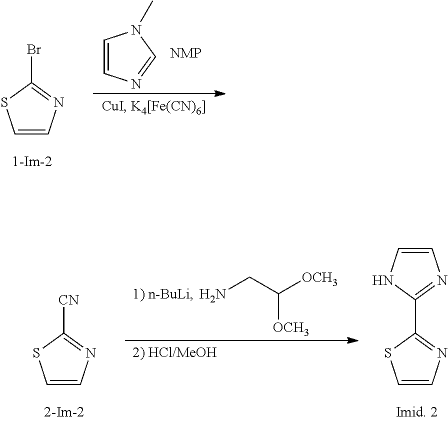

2-Bromothiazole (13.0 g, 1.0 eq), 1-methyl-imidazole (2.0 eq), CuI (0.05 eq) and K₄[Fe(CN)₆] (0.1 eq) were combined in 80 mL of dry NMP and heated in a sealed tube at 140° C. for 16 h. This mixture was extracted with EtOAc and solvent was removed from the organic fraction to give thiazole-2-carbonitrile (compound 2-Im-2).

A 2.5 M solution of nBuLi (2.0 eq) in hexane was added under argon to a solution of 2,2-dimethoxyethanamine (2.0 eq) in THF at −78° C. After stirring for 30 min, thiazole-2-carbonitrile (2-Im-2, 3.0 g, 1.0 eq) was added and the resulting solution was stirred at 0° C. for 2 h, then quenched with 20 mL of 5% MeOH in water. The volatiles were removed and 6N HCl was added to adjust to pH=1. The acidic solution was refluxed overnight, cooled to rt then poured into a mixture of ice and aqueous Na₂CO₃. This was extracted with EtOAc and the organic layer was concentrated to give Imadazole 2. LCMS (0.01% Ammonia): 152.1 m/z (M+H)⁺; ¹H-NMR (DMSO-d6, 500 MHz): δ: 13.19 (bs, 1H), 7.98 (d, 1H, J=3.0 Hz), 7.82 (d, 1H, J=3.0 Hz), 7.36 (s, 1H), 7.14 (s, 1H).

### Synthesis of 2-(1H-imidazol-2-yl)pyrimidine (Imidazole 3)

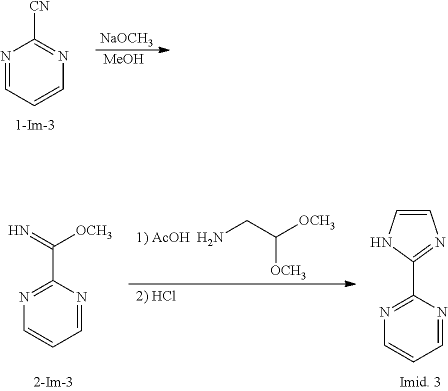

To a solution of NaOCH₃ (270 mg) in 50 mL of MeOH, pyrimidine-2-carbonitrile (1-Im-3, 50 mmol) was added. The mixture was stirred at rt for 1 h, then 2,2-dimethoxyethanamine (50 mmol) was added followed by 2 mL of AcOH. This mixture was stirred for 1 h, then 6N HCl was added to adjust pH=1. The resulting acidic solution was heated at reflux for 18 h. After cooling to rt, the reaction was poured into a mixture of ice and aqueous Na₂CO₃ solution, then extracted with EtOAc and the organic layer was concentrated to give 2-(1H-imidazol-2-yl)pyrimidine (Imidazole 3). LCMS (0.01% Ammonia): 147.2 m/z (M+H)⁺; ¹H-NMR (DMSO-d6, 500 MHz): δ: 13.04 (bs, 1H), 8.87 (d, 2H, J=5.0 Hz), 7.44 (t, 1H, J=5.0 Hz), 7.24 (s, 2H).

### 2-(4-(trifluoromethyl)phenyl)-1H-imidazole (Imidazole 4), 2-(4-(trifluoromethoxy)phenyl)-1H-imidazole (Imidazole 5), 2-(3-(trifluoromethoxy)phenyl)-1H-imidazole (Imidazole 6, and) 2-(1H-imidazol-2-yl)pyrazine (Imidazole 7)

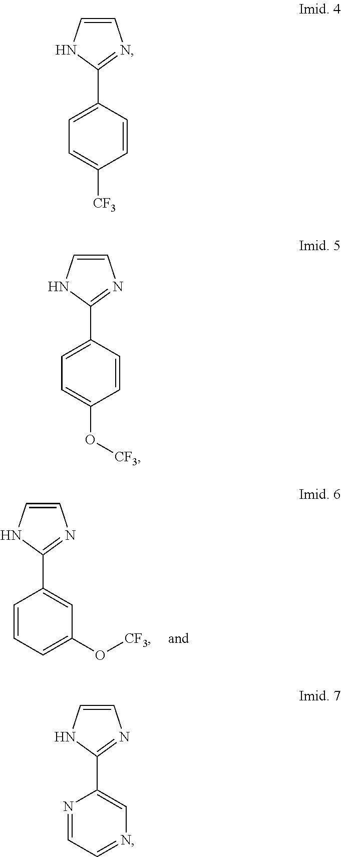

The Imidazoles 4, 5, 6 and 7 were prepared similarly to the methods used for the synthesis of Imidazole 3, with 4-(trifluoromethyl)benzonitrile, 4-(trifluoromethoxy)benzonitrile, 3-(trifluoromethoxy)benzonitrile, and pyrazine-2-carbonitrile, respectively, instead of pyrimidine-2-carbonitrile in the first step. Imidazole 4; LCMS (0.05% TFA): 213.1 m/z (M+H)⁺; ¹H-NMR (DMSO-d6, 500 MHz): 6:12.82 (bs, 1H), 8.15 (d, 2H, J=8.5 Hz), 7.82 (d, 2H, J=8.5 Hz), 7.35 (s, 1H), 7.12 (s, 1H). Imidazole 5; LCMS (0.01% Ammonia): 229.1 m/z (M+H)⁺; ¹H-NMR (DMSO-d6, 500 MHz): δ: 12.68 (bs, 1H), 8.07 (m, 2H), 7.46 (d, 2H, J=8.5 Hz), 7.19 (bs, 2H). Imidazole 6; LCMS (0.01% Ammonia): 229.1 m/z (M+H)⁺; ¹H-NMR (DMSO-d6, 500 MHz): δ: 12.73 (bs, 1H), 7.97 (d, 1H, J=8.0 Hz), 7.90 (s, 1H), 7.59 (t, 1H, J=8.0 Hz), 7.33 (d, 2H, J=8.0 Hz), 7.07 (s, 1H). Imidazole 7; LCMS (0.01% Ammonia): 147.2 m/z (M+H)⁺; ¹H-NMR (DMSO-d6, 500 MHz): δ: 13.19 (bs, 1H), 9.34 (d, 1H, J=1.5 Hz), 8.70 (dd, 1H, J¹=3 Hz, J²=1.5 Hz), 8.65 (d, 1H, J=3 Hz), 7.34 (bs, 2H).

### Synthesis of 3-(1H-imidazol-2-yl)pyridazine (Imidazole 8)

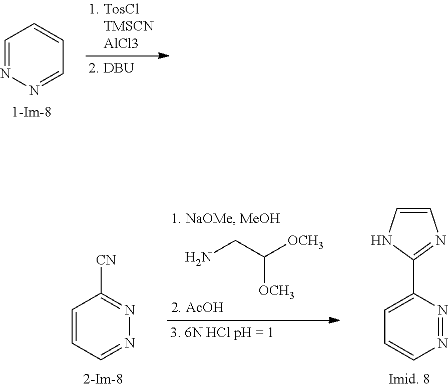

The mixture of pyridazine (1-Im-8, 1 eq), TMSCN (1.8 eq) and A1Cl₃ (0.01 eq) in dry DCM was stirred for 1 h under Ar at 0° C., then TosC1 (1.72 eq) was added. The resulting mixture was stirred for 48 h under Ar at rt. The solvent was removed under reduced pressure, then the residue was treated with EtOH and the reaction was filtered give a solid. The solid was added to dry THF, then DBU (1.2 eq) was added to the mixture. The mixture was stirred for 2 h under Ar at rt, then aqueous NH₄Cl was added and the mixture was extracted with EtOAc, the organic layer was dried with Na₂SO₄, concentrated and the residue was purified by silica column chromatography to give pyridazine-3-carbonitrile (compound 2-Im-8).

pyridazine-3-carbonitrile (compound 2-Im-8, 1 eq) was added to NaOMe (0.5 eq) in MeOH and stirred for 3 h at rt, then 2,2-dimethoxyethanamine (1 eq) and AcOH (2 eq) were added to the mixture and stirred for 2 h under Ar at 50° C. After this time, 6N HCl was added to the mixture to adjust to pH=1; the mixture was heated to reflux for 18 h, then cooled to rt. The solvent was removed and the residue was treated with aqueous Na₂CO₃ to give a mixture at pH=10. The resulting solid was collected by filtration and washed with PE to give Imidazole 8. LCMS (0.01% Ammonia): 147.1 m/z (M+H)⁺; ¹H-NMR (DMSO-d6, 500 MHz): δ: 13.37 (bs, 1H), 9.21 (d, 1H, J=5.0 Hz), 8.24 (d, 1H, J=8.5 Hz), 7.79 (dd, 1H, J¹=8.5 Hz, J²=5.0 Hz), 7.37 (s, 1H), 7.19 (s, 1H).

### Synthesis of 1-(1H-imidazol-2-yl)isoquinoline (Imidazole 9)

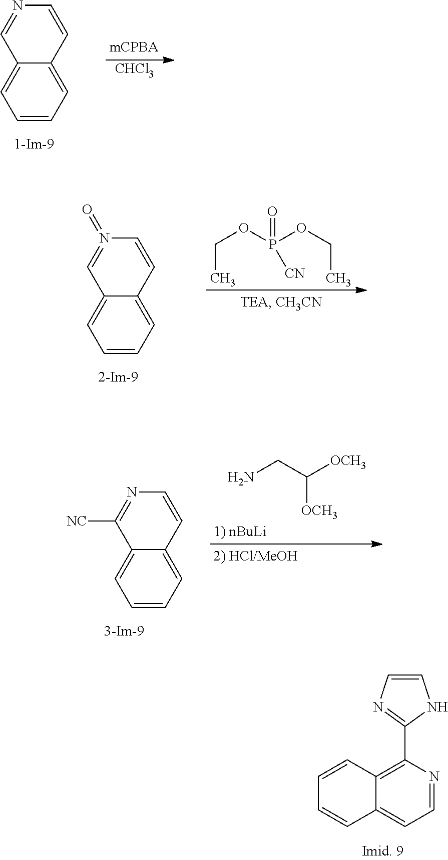

To a stirred solution of isoquinoline (1-Im-9, 5 g, 1.0 eq) in 50 mL of CHCl₃, mCPBA (2.0 eq) was added. The mixture was stirred at rt for 1 h. The reaction was washed with 5% aqueous Na₂SO₃ and aqueous Na₂CO₃, then concentrated and the residue was purified by silica column chromatography to give isoquinoline 2-oxide (2-Im-9).

To a stirred solution of isoquinoline 2-oxide (2-Im-9, 5.8 g) in 140 mL of acetonitrile, diethyl phosphoro-cyanidate (1.5 eq) was added under argon followed by slow addition of TEA (3.0 eq). The mixture was refluxed for 18 h and then extracted with DCM. The organic layer was concentrated and purified by silica column chromatography to give isoquinoline-1-carbonitrile (3-Im-9).

nBuLi (2.5 M in hexane, 2.0 eq) was added under argon to a solution of 2,2-dimethoxyethanamine (2.0 eq) in THF at −78° C. After stirring for 30 min, isoquinoline-1-carbonitrile (3-Im-9, 3.0 g, 1.0 eq) was added. The resulting solution was stirred at 0° C. for 2 h. The reaction was quenched with 20 mL of 5% MeOH in water, the volatiles were removed, then 6N HCl was added to adjust to pH=1. The acidified solution was refluxed 18 h, then cooled to rt and poured into ice/Na₂CO₃ solution. This was extracted with EtOAc and concentrated to provide Imidazole 9. LCMS (0.01% Ammonia): 196.1 m/z (M+H)⁺; ¹H-NMR (DMSO-d6, 500 MHz): δ: 12.93 (bs, 1H), 9.92 (d, 1H, J=8.0 Hz), 8.51 (d, 1H, J=5.5 Hz), 7.96 (d, 1H, J=8.0 Hz), 7.79 (d, 1H, J=5.5 Hz), 7.76 (t, 1H, J=8.0 Hz), 7.70 (t, 1H, J=8.0 Hz), 7.30 (s, 1H), 7.21 (s, 1H).

### Synthesis of 3-(1H-imidazol-2-yl)quinoline (Imidazole 10)

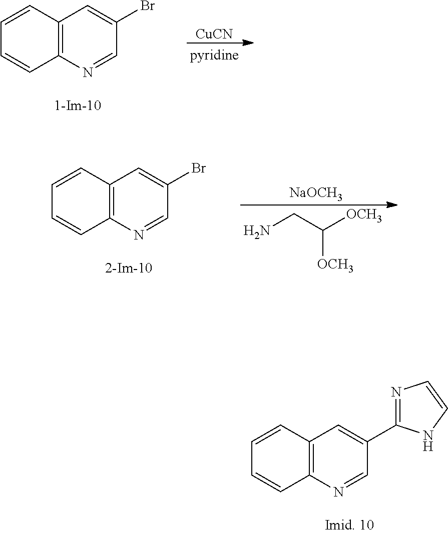

A suspension of 3-bromoquinoline (1-Im-10, 1.5 g) and CuCN (3 eq) in 10 mL of pyridine in a 25 mL microwave tube was heated at 250° C. for 30 min in a microwave. This was repeated 10 times and the reactions were combined and diluted with 200 mL of EtOAc. The solids were removed by filtration and the EtOAc solution concentrated. The residue was taken up in a solution prepared from 80 mL of 30% aqueous NH₃ and 800 mL of water. This was extracted with EtOAc (4×800 mL) then the combined extracts were dried with anhydrous Na₂SO₄, concentrated and purified by silica gel chromatography (PE: EtOAc=3:1) to give quinoline-3-carbonitrile (2-Im-10).

Quinoline-3-carbonitrile (2-Im-10, 10 g) was suspended in 65 mL of MeOH, then NaOCH₃ (0.1 eq) was added and the reaction was stirred at 25° C. for 15 h. 2,2-Dimethoxyethanamine (1 eq) was added, followed by acetic acid (2 eq) and the mixture was heated at 50° C. for 1 h. The reaction was cooled to rt and 30 mL of 6N HCl was added to give a pH=1 and this mixture was heated at reflux for 5 h. The reaction was diluted with 200 mL of water and extracted with EtOAc (2×200 mL). The aqueous phase was made basic (pH=10) with solid sodium carbonate and the desired compound precipitated out and was isolated by filtration and washed with water to give Imidazole 10. LCMS (0.01% Ammonia): 196.2 m/z (M+H)⁺; ¹H-NMR (DMSO-d6, 500 MHz): δ: 12.92 (bs, 1H), 9.51 (d, 1H, J=2.0 Hz), 8.78 (d, 1H, J=2.0 Hz), 8.03 (dd, 2H, J=8.5 Hz), 7.77 (t, 1H, J=8.0 Hz), 7.65 (t, 1H, J=8.0 Hz), 7.28 (bs, 2H).

### Synthesis of 2-(4-isopropylphenyl)-1H-imidazole (Imidazole 11)

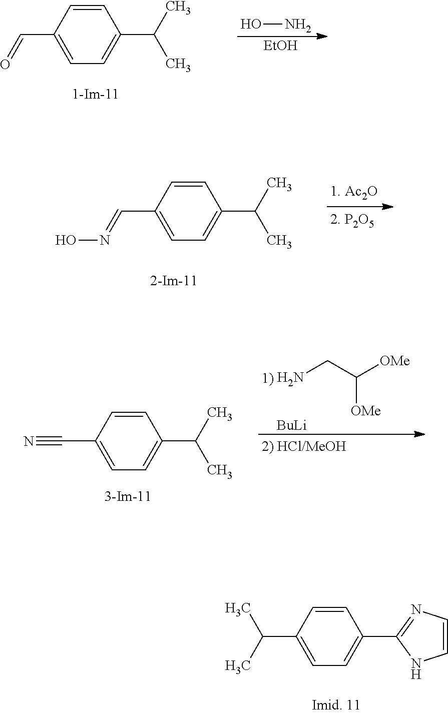

To a solution of compound 1-Im-11 (14.8 g, 1.0 eq) in 148 mL of EtOH, hydroxylamine hydrochloride (1.0 eq) was added. The reaction mixture was stirred at rt for 1 h and concentrated to give compound 2-Im-11.

Compound 2-Im-11 (13.04 g, 1.0 eq) was dissolved in 40 mL of Ac₂O and refluxed for 3 h, then cooled to room temperature and P₂O₅ (800 mg) was added; the resulting mixture was refluxed for another 30 min. This was extracted with a mixture of 9:1 PE:EtOAc and purified by silica column chromatography to give compound 3-Im-11.

n-BuLi (2.5M in hexane, 2.0 eq) was added under argon to a solution of dimethoxyethanamine (2.0 eq) in THF at −78° C. This was stirred for 30 min at −78° C., then compound 3-Im-11 (3.0 g, 1.0 eq) was added. The resulting solution was stirred at 0° C. for 2 h, then quenched with 5% MeOH/H₂O. The solvent was removed and then HCl (6N) was added until pH=1; this mixture was refluxed for 18 h, then the reaction was cooled to room temperature and poured into ice/aq. Na₂CO₃ mixture, extracted with EtOAc and purified by silica column chromatography to provide Imidazole 11. LCMS (0.05% TFA): 187.2 m/z (M+H)⁺; ¹H-NMR (DMSO-d6, 500 MHz): δ: 12.41 (bs, 1H), 7.85 (d, 2H, J=8.0 Hz), 7.30 (d, 2H, J=8.0 Hz), 7.10 (bs, 2H), 2.91 (m, 1H), 1.19 (d, 6H, J=18.5 Hz).

### 2-(3-isopropylphenyl)-1H-imidazole (Imidazole 12)

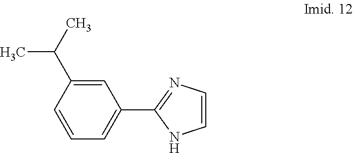

Imidazole 12 was prepared similarly to the method used for Imidazole 11 with 3-isopropylbenzaldehyde instead of 4-isopropylbenzaldehyde. LCMS (0.05% TFA): 187.2 m/z (M+H)⁺; ¹H-NMR (CDCl₃, 500 MHz): δ: 13.21 (bs, 1H), 7.85 (s, 1H), 7.77 (d, 1H, J=8.0 Hz), 7.21 (t, 1H, J=8.0 Hz), 7.16 (s, 2H), 7.14 (t, 1H, J=8.0 Hz), 2.72 (m, 1H), 1.05 (d, 6H, J=7.0 Hz).

### Preparation of Boronic Acids

### 5-(thiazol-2-yl)-1-((2-(trimethylsilyl)ethoxy)methyl)-1H-pyrazol-4-ylboronic acid (Boronic Acid 1)

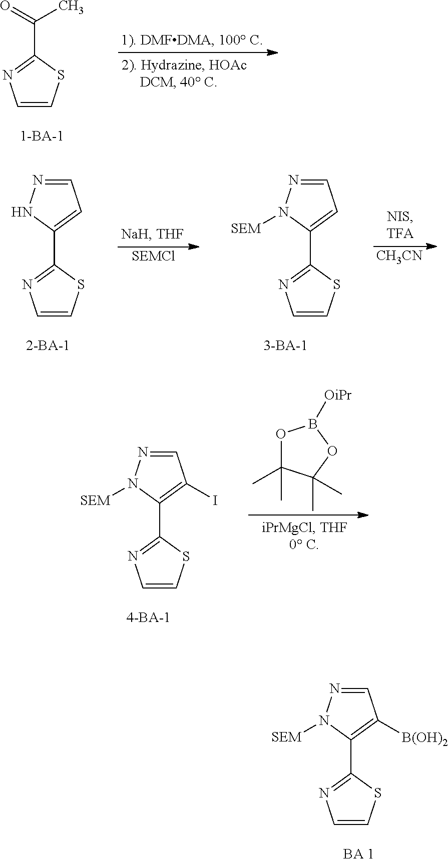

Dissolved 1-(thiazol-2-yl)ethanone (1-BA-1, 5 g, 39.7 mmole) in DMF.DMA (9.5 g, 2 eq). The resulting mixture was warmed to 100° C. until all the ketone starting material was consumed. This material was concentrated under reduced pressure to give 6.5 g of crude intermediate. This material was dissolved in 25 mL of DCM and 5 mL of HOAc was added, followed by hydrazine (5 g, 4 eq) at 0° C. The resulting mixture was heated at reflux until all the starting material was consumed. The reaction mixture was cooled to rt and neutralized with 30 mL of a saturated NaHCO₃ solution. The layers were separated and the aqueous layer was extracted with DCM (2×50 mL). The organic layers were dried over MgSO₄, filtered, and concentrated under reduced pressure. The crude material was purified by MPLC (eluted with 0-20% MeOH/DCM) to give 2-(1H-pyrazol-5-yl)thiazole (Compound 2-BA-1, ˜6 g). LC/MS: 152.0 m/z (M+H)⁺.

To a stirring mixture of 2-(1H-pyrazol-5-yl)thiazole (Compound 1-BA-1, 6.5 g) in 50 mL of THF, NaH (1.8 g, 43 mmole, 60% by weight) was added in portions. The reaction mixture was stirred at rt for 20 min before SEM-Cl (7.8 g, 47.3 mmole) was added dropwise. The reaction mixture was stirred at rt until all the starting material was consumed. The crude reaction mixture was slowly quenched with 50 mL of water, 50 mL of brine, and diluted with 50 mL of EtOAc. The layers were separated and the aqueous layer was extracted with EtOAc (2×50 mL). The organic layer was concentrated and purified by MPLC [0-50% EtOAc/hex] to give 2-(1-((2-(trimethylsilyl)ethoxy)methyl)-1H-pyrazol-5-yl)thiazole (Compound 3-BA-1, 11.3 g). LCMS: 282.1 m/z (M+H)⁺.

To a stirring mixture of 2-(1-((2-(trimethylsilyl)ethoxy)methyl)-1H-pyrazol-5-yl)thiazole (Compound 2-BA-1) in 50 mL of acetonitrile at rt under nitrogen were added TFA (1 mL) and NIS (10.8 g). The reaction mixture was stirred at rt overnight and an additional amount of NIS (0.5 eq to 1.0 eq) was added as needed. The crude reaction mixture was slowly quenched with ˜30 mL of a saturated aqueous Na₂S₂O₃ solution, and ˜30 mL of a saturated aqueous NaHCO₃ solution. The reaction mixture was diluted with 50 mL of EtOAc, the layers were separated and the aqueous layer was extracted with EtOAc (2×50 mL). The organic layer was purified by MPLC (eluted with 0-50% EtOAc/hex) to give 2-(4-iodo-1-((2-(trimethylsilyl)ethoxy)methyl)-1H-pyrazol-5-yl)thiazole (Compound 4-BA-1). LCMS: 408.0 m/z (M+H)⁺.

To stirring mixture of 2-(4-iodo-1-((2-(trimethylsilyl)ethoxy)methyl)-1H-pyrazol-5-yl)thiazole (Compound 4-BA-1, 11.3 g) in THF (0.35 M) at 0° C., a solution of iPrMgCl (16 mL, 1.2 eq) in THF was added dropwise. The reaction mixture was stirred for 30 min before 2-isopropoxy-4,4,5,5-tetramethyl-1,3,2-dioxaborolane (9.1 mL, 1.6 eq) was added over 10 min. The cold bath was removed and the resulting mixture was stirred at rt for 1 hr. The mixture was diluted with 50 mL of EtOAc and quenched with 25 mL of a saturated aqueous NH₄Cl solution. The layers were separated and the aqueous layer was extracted with EtOAc. The organic portion was purified by MPLC (eluted with 0-100% EtOAc/Hex) to give Boronic Acid 1. LCMS: 326.1 m/z (M+H)⁺.

5-(pyridin-2-yl)-1-((2-(trimethylsilyl)ethoxy)methyl)-1H-pyrazol-4-ylboronic acid (Boronic Acid 3) and 5-(2,4-difluorophenyl)-4-(4,4,5,5-tetramethyl-1,3,2-dioxaborolan-2-yl)-1-(2-(trimethylsilyl)ethoxy)methyl)-1H-pyrazole (Boronic Acid 4)

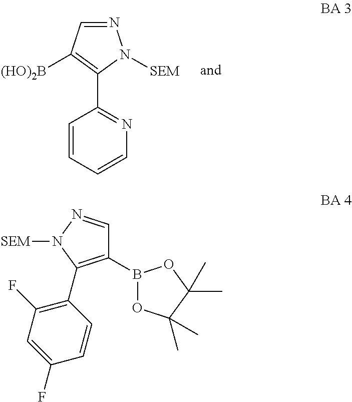

are prepared similarly with 1-(pyridin-2-yl)ethanone and 1-(2,4-difluorophenyl)ethanone, respectively, instead of 1-(thiazol-2-yl)ethanone in the first step. Boronic Acid 4 is isolated and used as the dioxaborolane ester.

### Preparation of 3-phenyl-4-(4,4,5,5-tetramethyl-1,3,2-dioxaborolan-2-yl)pyridine (Boronic Acid 2)

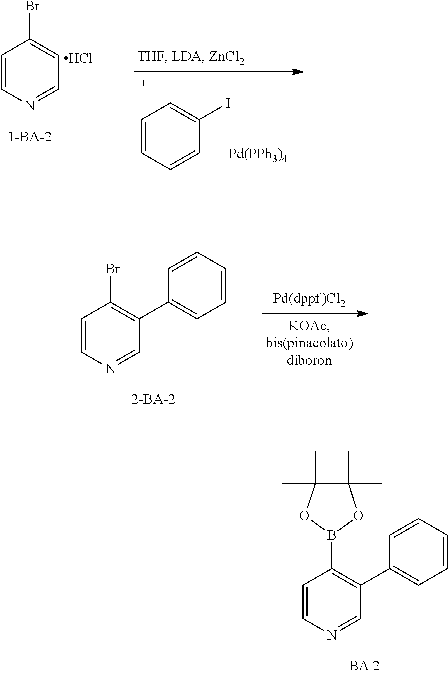

4-bromopyridine hydrochloride (1-BA-2, 1 g, 5.14 mmol) was dissolved in 5.1 mL of THF and the resulting solution was cooled to −78° C. LDA (10.28 mL of a 1 M solution in THF) was added over 10 minutes and the reaction mixture became brown. After stirring for 30 minutes, ZnCl₂ (10.3 mL of a 0.5 M solution in THF) was added over 10 minutes and the resulting mixture was stirred for 10 minutes and then allowed to warm to rt. Iodobenzene (0.229 mL, 2.06 mmol) and Pd(PPh₃)₄ (593 mg, 0.514 mmol) were added and the resulting mixture was stirred under reflux for 2 h. The reaction mixture was diluted with aqueous saturated ammonium chloride and extracted with ethyl acetate. The organic phase was dried over Na₂SO₄ and evaporated. The residue was purified by silica column (hexane:EtOAc) to give 4-bromo-3-phenylpyridine (2-BA-2, 741 mg, 62%); LCMS: 234.0 m/z (M+H)⁺.

4-bromo-3-phenylpyridine (2-BA-2, 0.11 mg, 0.469 mmol), Pd(dppf)Cl₂ (34 mg, 0.0469 mmol), KOAc (138 mg, 1.41 mmol) and bis(pinacolato)diboron (238 mg, 0.939 mmol) were dissolved in 1.5 mL of DMF and a stream of nitrogen was bubbled through the solution for 5 minutes. The resulting solution was stirred at 90° C. for 18 hours and was subsequently diluted with ethyl acetate and washed with brine. The organic phase was dried over Na₂SO₄ and evaporated to give Boronic Acid 2 (741 mg, 62%); LCMS: 282.2 m/z (M+H)⁺.

### Example 1

### Synthesis of (R)-5-Cyclobutyl-7-(2-(3,4-difluorophenyl)-1H-imidazol-1-yl)-4-ethyl-4,5-dihydro-[1,2,4]triazolo[4,3-f]pteridine

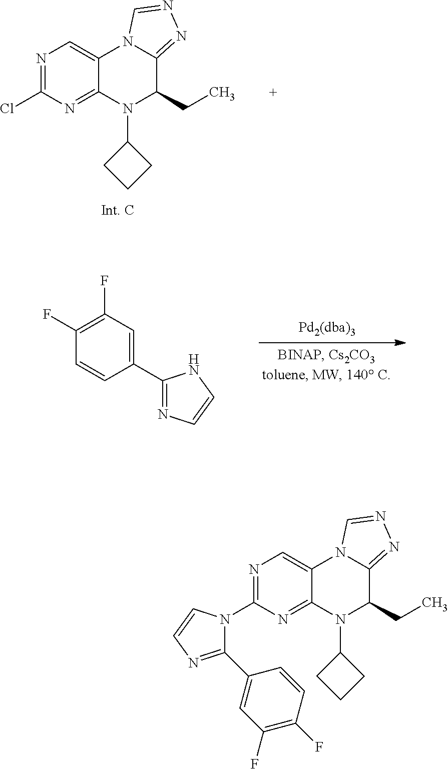

To Intermediate C ((R)-7-chloro-5-cyclobutyl-4-ethyl-4,5-dihydro-[1,2,4]triazolo[4,3-f]pteridine, 20 mg, 1 EQ) in toluene (1 mL), Pd₂(dba)₃ (25.3 mg, 0.4 EQ), BINAP (34.4 mg, 0.8 EQ), Cs₂CO₃ (68 mg, 3 EQ), and 2-(3,4-difluorophenyl)-1H-imidazole (15 mg, 1.2 EQ) were added. The reaction mixture was flushed with argon twice and heated under microwave condition at 150° C. overnight. The mixture was concentrated and water was added to the residue, then extracted with EtOAc. The EtOAc layer was separated and dried with anhydrous Na₂SO₄. The crude material was purified via isco column and further purified via HPLC to afford the desired product. LCMS: [M+H] 435.2; ¹H-NMR (CDCl₃, 300 MHz): δ 8.76 (s, 1H), 8.37 (s, 1H), 7.93 (s, 1H), 7.61 (s, 1H), 7.45-7.31 (m, 3H), 5.49-5.15 (m, 1H), 4.02-3.94 (m, 1H), 2.31-1.62 (m, 8H), 0.86 (t, J=7.33 Hz, 3H).

Additional compounds are prepared similarly to this method, optionally replacing Intermediate C with a suitable intermediate, and/or replacing 2-(3,4-difluorophenyl)-1H-imidazole with an appropriate compound. The following compounds are prepared:
(R)-5-cyclobutyl-7-(2-(3,4-difluorophenyl)-1H-imidazol-1-yl)-4-ethyl-1-methyl-4,5-dihydro-[1,2,4]triazolo[4,3-f]pteridine (Example 2),
(R)-5-Cyclobutyl-4-ethyl-7-(2-phenyl-1H-imidazol-1-yl)-4,5-dihydro-[1,2,4]triazolo[4,3-f]pteridine (Example 3),
(R)-5-Cyclobutyl-4-ethyl-7-(2-phenyl-1H-imidazol-1-yl)-4,5-dihydro-[1,2,4]triazolo[4,3-f]pteridine (Example 4),
(R)-5-cyclobutyl-4-ethyl-7-(2-(2-fluorophenyl)-1H-imidazol-1-yl)-4,5-dihydro-[1,2,4]triazolo[4,3-f]pteridine (Example 5),
(R)-5-cyclobutyl-4-ethyl-7-(2-(2-fluorophenyl)-1H-imidazol-1-yl)-1-methyl-4,5-dihydro-[1,2,4]triazolo[4,3-f]pteridine (Example 6),
(R)-5-Cyclobutyl-4-ethyl-7-(2-(4-fluorophenyl)-1H-imidazol-1-yl)-4,5-dihydro-[1,2,4]triazolo[4,3-f]pteridine (Example 7),
(R)-5-cyclobutyl-4-ethyl-7-(2-(4-fluorophenyl)-1H-imidazol-1-yl)-1-methyl-4,5-dihydro-[1,2,4]triazolo[4,3-f]pteridine (Example 8),
(R)-5-cyclobutyl-7-(2-(2,3-difluorophenyl)-1H-imidazol-1-yl)-4-ethyl-4,5-dihydro-[1,2,4]triazolo[4,3-f]pteridine (Example 9),
(R)-5-cyclobutyl-7-(2-(2,3-difluorophenyl)-1H-imidazol-1-yl)-4-ethyl-1-methyl-4,5-dihydro-[1,2,4]triazolo[4,3-f]pteridine (Example 10),
(R)-5-Cyclobutyl-7-(2-(2,4-difluorophenyl)-1H-imidazol-1-yl)-4-ethyl-4,5-dihydro-[1,2,4]triazolo[4,3-f]pteridine (Example 11),
(R)-5-cyclobutyl-7-(2-(2,4-difluorophenyl)-1H-imidazol-1-yl)-4-ethyl-1-methyl-4,5-dihydro-[1,2,4]triazolo[4,3-f]pteridine (Example 12).

The following table provides the example number (column 1), Intermediate used (column 2) and ring reactant used (column 3) to give the compound shown in column 4. Identification data is provided in column 5.

| Ex. | | | | |
|---|---|---|---|---|
| No. | Int. | Ring reactant | Compound structure | Identification |
| 2 | D | 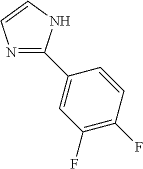 | 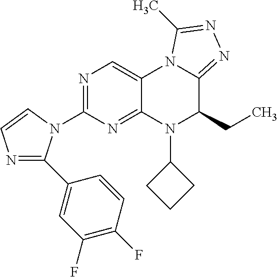 | |
| 3 | C | 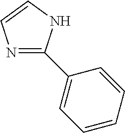 | 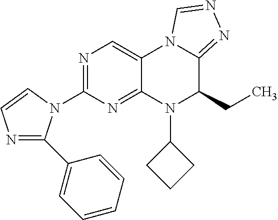 | LCMS: [M + H] 399.2; ¹H-NMR (CDCl₃, 300 MHz): δ: 8.68 (s, 1H), 8.37 (s, 1H), 7.83-7.82 (m, 1H), 7.58-7.43 (m, 6H), 5.21-5.17 (m, 1H), 3.77-3.66 (m, 1H), 2.11-1.47 (m, 8H), 0.89 (t, J = 7.0 Hz, 3H) |
| 4 | D | 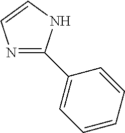 | 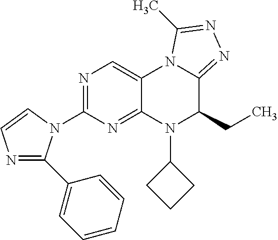 | |
| 5 | C | 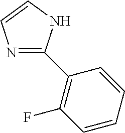 | 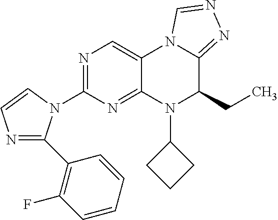 | LC-MS: [M + H] 417.2; ¹H-NMR (CDCl₃, 300 MHz): δ: 8.68 (s, 1H), 8.33 (s, 1H), 7.96 (d, J = 1.8 Hz, 1H), 7.91-7.86 (m, 1H), 7.62-7.55 (m, 2H), 7.44-7.39 (m, 1H), 7.11-7.05 (m, 1H), 5.41-5.18 (m, 1H), 3.71-3.61 (m, 1H), 2.15-1.42 (m, 8H), 0.75 (t, J = 9 Hz, 3H) |
| 6 | D | 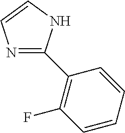 | 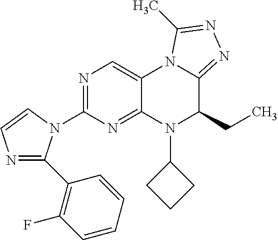 | |
| 7 | C | 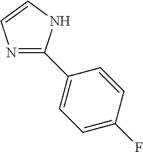 | 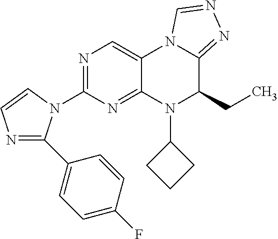 | LCMS: [M + H] 417.1; ¹H-NMR (CDCl₃, 300 MHz): δ: 8.73 (s, 1H), 8.38 (s, 1H), 8.18 (b s, 1H), 7.90 (s, 1H), 7.63-7.59 (m, 2H), 7.23-7.18 (m, 2H), 5.26-5.25 (m, 1H), 3.90-3.81 (m, 1H), 2.14-1.54 (m, 8H), 0.81 (t, J = 7.2 Hz, 3H) |
| 8 | D | 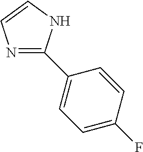 | 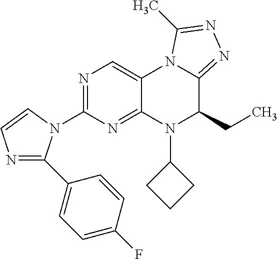 | |
| 9 | C | 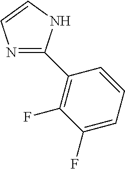 | 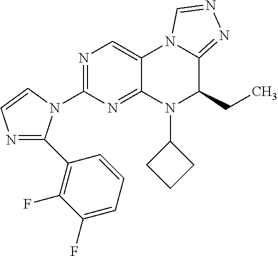 | LCMS: [M + H] 435.1; ¹H-NMR (CDCl₃, 300 MHz): δ: 8.74 (s, 1H), 8.36 (s, 1H), 8.03 (s, 1H), 7.67-7.61 (m, 2H), 7.49-7.34 (m, 2H), 5.30-5.26 (m, 1H), 3.86-3.75 (m, 1H), 2.20-1.48 (m, 8H), 0.78 (t, J = 8.13 (Hz, 3H) |
| 10 | D | 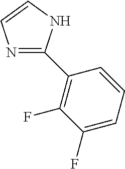 | 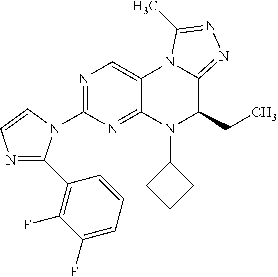 | |
| 11 | C | 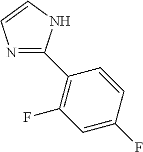 | 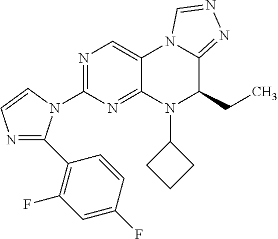 | LCMS: [M + H] 435.1; ¹H-NMR (CDCl₃, 300 MHz): δ: 8.69 (s, 1H), 8.32 (s, 1H), 7.98 (s, 1H), 7.93-7.85 (m, 1H), 7.59 (s, 1H), 7.19-7.13 (m, 1H), 6.89-6.84 (m, 1H), 5.28-5.26 (m, 1H), 3.94-3.83 (m, 1H) 2.20-1.55 (m, 8H), 0.81 (t, J = 7.32 Hz, 3H) |
| 12 | D | 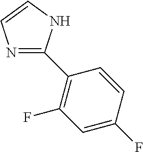 | 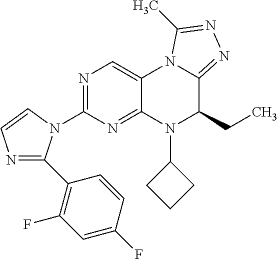 | |

### Example 13

### Synthesis of (R)-5-cyclopentyl-4-ethyl-7-(pyrrolidin-1-yl)-4,5-dihydro-[1,2,4]triazolo[4,3-f]pteridine

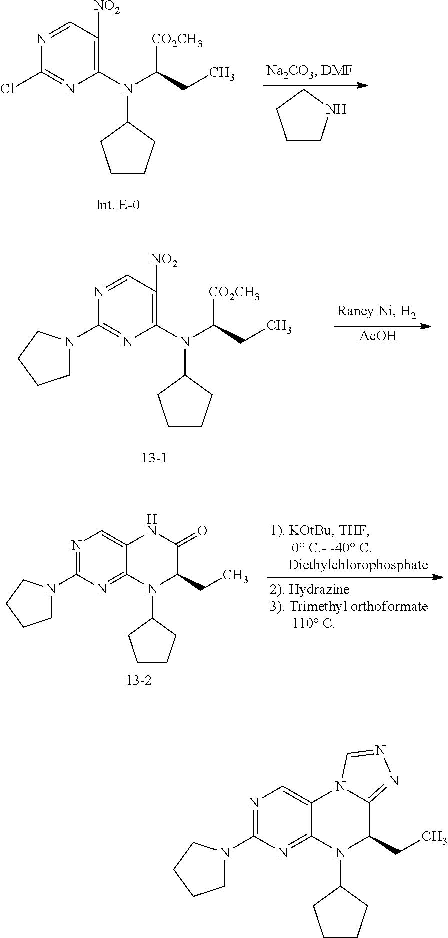

To a solution of (R)-methyl 2-((2-chloro-5-nitropyrimidin-4-yl)(cyclopentyl)amino)butanoate (Intermediate E-0) in DMF, Na₂CO₃ (1 eq) and pyrrolidine (1.6 eq) are added. The mixture is stirred at 100° C. for 3 hr under N₂, then is diluted with water and extracted with EtOAc. The solvent is removed by evaporation and the residue is purified by silica column to give (R)-methyl 2-(cyclopentyl(5-nitro-2-(pyrrolidin-1-yl)pyrimidin-4-yl)amino)butanoate (13-1).

To a solution of (R)-methyl 2-(cyclopentyl(5-nitro-2-(pyrrolidin-1-yl)pyrimidin-4-yl)amino)butanoate (13-1) in AcOH, Raney Ni is added and the mixture is stirred under H₂ at 75° C. for 5 hr until the starting material is consumed. The solvent is removed and the residue is purified by flash silica column to give (R)-8-cyclopentyl-7-ethyl-2-(pyrrolidin-1-yl)-7,8-dihydropteridin-6(5H)-one (13-2).

A solution of (R)-8-cyclopentyl-7-ethyl-2-(pyrrolidin-1-yl)-7,8-dihydropteridin-6(5H)-one (13-2) in THF is stirred at −20° C. and potassium tert-butoxide (1.3 eq) is added over 5 min. The reaction mixture is warmed up to 0° C. for 25 min after complete addition. The reaction mixture is cooled to −40° C. and diethylchlorophosphate (1.4 eq) is added. The reaction mixture is warmed up to rt for 45 min. To the resulting mixture, 1M hydrazine (10 eq) is added and the reaction mixture is stirred at rt for 18 h. The reaction mixture is concentrated under reduced pressure and diluted with DCM and a saturated NaHCO₃ solution. The organic layer is dried over MgSO₄ and concentrated under pressure. The resulting material is purified via the iso column, then dissolved in trimethyl orthoformate (10 eq) and heated to 110° C. for 1 h. The reaction mixture is concentrated under reduced pressure and purified via silica gel column chromatography to afford the title compound.

Additional compounds are prepared similarly to this method, optionally replacing Intermediate E-0 with a suitable intermediate, and/or replacing pyrrolidine with an appropriate ring reactant, and/or replacing trimethyl orthoformate with trimethyl orthoacetate in the final step. In some instances, where a racemic mixture results, the two enantiomers may be isolated by chiral chromatography. The following compounds are prepared:
(R)-5-cyclopentyl-4-ethyl-1-methyl-7-(pyrrolidin-1-yl)-4,5-dihydro-[1,2,4]triazolo[4,3-f]pteridine (Example 14),
(R)-5-cyclopentyl-4-ethyl-7-(piperidin-1-yl)-4,5-dihydro-[1,2,4]triazolo[4,3-f]pteridine (Example 15),
(R)-5-cyclopentyl-4-ethyl-1-methyl-7-(piperidin-1-yl)-4,5-dihydro-[1,2,4]triazolo[4,3-f]pteridine (Example 16),
(R)-5-cyclopentyl-4-ethyl-7-(1H-imidazol-1-yl)-4,5-dihydro-[1,2,4]triazolo[4,3-f]pteridine (Example 17),
(R)-5-cyclopentyl-4-ethyl-7-(1H-imidazol-1-yl)-1-methyl-4,5-dihydro-[1,2,4]triazolo[4,3-f]pteridine (Example 18),
(R)-7-(1H-benzo[d]imidazol-1-yl)-5-cyclopentyl-4-ethyl-4,5-dihydro-[1,2,4]triazolo[4,3-f]pteridine (Example 19),
R)-7-(1H-benzo[d]imidazol-1-yl)-5-cyclopentyl-4-ethyl-1-methyl-4,5-dihydro-[1,2,4]triazolo[4,3-f]pteridine (Example 20),
(R)-ethyl 1-(5-cyclopentyl-4-ethyl-4,5-dihydro-[1,2,4]triazolo[4,3-f]pteridin-7-yl)-1H-pyrazole-4-carboxylate (Example 45),
(R)-ethyl 1-(5-cyclopentyl-4-ethyl-1-methyl-4,5-dihydro-[1,2,4]triazolo[4,3-f]pteridin-7-yl)-1H-pyrazole-4-carboxylate (Example 46),
(R)-4-ethyl-7-(1H-imidazol-1-yl)-5-isopropyl-4,5-dihydro-[1,2,4]triazolo[4,3-f]pteridine (Example 53),
(R)-4-ethyl-7-(1H-imidazol-1-yl)-5-isopropyl-1-methyl-4,5-dihydro-[1,2,4]triazolo[4,3-f]pteridine (Example 54),
(R)-7-(1H-imidazol-1-yl)-11,12,13,13a-tetrahydro-10H-pyrido[2,1-h][1,2,4]triazolo[4,3-f]pteridine (Example 57),
(R)-7-(1H-imidazol-1-yl)-3-methyl-11,12,13,13a-tetrahydro-10H-pyrido[2,1-h][1,2,4]triazolo[4,3-f]pteridine (Example 58),
(R)-5-cyclopentyl-4-ethyl-7-(1H-pyrazol-1-yl)-4,5-dihydro-[1,2,4]triazolo[4,3-f]pteridine (Example 67),
(R)-5-cyclopentyl-4-ethyl-1-methyl-7-(1H-pyrazol-1-yl)-4,5-dihydro-[1,2,4]triazolo[4,3-f]pteridine (Example 68),
(R)-7-(1H-imidazol-1-yl)-10,11,12,12a-tetrahydropyrrolo[2,1-h][1,2,4]triazolo[4,3-f]pteridine (Example 73),
(R)-7-(1H-imidazol-1-yl)-3-methyl-10,11,12,12a-tetrahydropyrrolo[2,1-h][1,2,4]triazolo[4,3-f]pteridine (Example 74),
(S)-12a-ethyl-7-(2-phenyl-1H-imidazol-1-yl)-10,11,12,12a-tetrahydropyrrolo[2,1-h][1,2,4]triazolo[4,3-f]pteridine (Example 99),
(S)-12a-ethyl-3-methyl-7-(2-phenyl-1H-imidazol-1-yl)-10,11,12,12a-tetrahydropyrrolo[2,1-h][1,2,4]triazolo[4,3-f]pteridine (Example 100),
(S)-12a-methyl-7-(2-phenyl-1H-imidazol-1-yl)-10,11,12,12a-tetrahydropyrrolo[2,1-h][1,2,4]triazolo[4,3-f]pteridine (Example 101),
(S)-3,12a-dimethyl-7-(2-phenyl-1H-imidazol-1-yl)-10,11,12,12a-tetrahydropyrrolo[2,1-h][1,2,4]triazolo[4,3-f]pteridine (Example 102),
(R)- 5-cyclopentyl-4-ethyl-7-(2-(pyridin-4-yl)-1H-imidazol-1-yl)-4,5-dihydro-[1,2,4]triazolo[4,3-f]pteridine (Example 103),
(R)-5-cyclopentyl-4-ethyl-1-methyl-7-(2-(pyridin-4-yl)-1H-imidazol-1-yl)-4,5-dihydro-[1,2,4]triazolo[4,3-f]pteridine (Example 104),
(S)-12a-ethyl-7-(2-(pyridin-4-yl)-1H-imidazol-1-yl)-10,11,12,12a-tetrahydropyrrolo[2,1-h][1,2,4]triazolo[4,3-f]pteridine (Example 105),
(S)-12a-ethyl-3-methyl-7-(2-(pyridin-4-yl)-1H-imidazol-1-yl)-10,11,12,12a-tetrahydropyrrolo[2,1-h][1,2,4]triazolo[4,3-f]pteridine (Example 106),
(S)-12a-ethyl-7-(2-(pyridin-3-yl)-1H-imidazol-1-yl)-10,11,12,12a-tetrahydropyrrolo[2,1-h][1,2,4]triazolo[4,3-f]pteridine (Example 107),
(S)-12a-ethyl-3-methyl-7-(2-(pyridin-3-yl)-1H-imidazol-1-yl)-10,11,12,12a-tetrahydropyrrolo[2,1-h][1,2,4]triazolo[4,3-f]pteridine (Example 108),
(S)-12a-ethyl-7-(2-(pyrazin-2-yl)-1H-imidazol-1-yl)-10,11,12,12a-tetrahydropyrrolo[2,1-h][1,2,4]triazolo[4,3-f]pteridine (Example 109),
(S)-12a-ethyl-3-methyl-7-(2-(pyrazin-2-yl)-1H-imidazol-1-yl)-10,11,12,12a-tetrahydropyrrolo[2,1-h][1,2,4]triazolo[4,3-f]pteridine (Example 110),
(S)-12a-ethyl-7-(2-(pyridin-2-yl)-1H-imidazol-1-yl)-10,11,12,12a-tetrahydropyrrolo[2,1-h][1,2,4]triazolo[4,3-f]pteridine (Example 111),
(S)-12a-ethyl-3-methyl-7-(2-(pyridin-2-yl)-1H-imidazol-1-yl)-10,11,12,12a-tetrahydropyrrolo[2,1-h][1,2,4]triazolo[4,3-f]pteridine (Example 112),
4-perdeuteroethyl-5-perdeuteroisopropyl-7-(2-(3-(trifluoromethoxy)phenyl)-1H-imidazol-1-yl)-4,5-dihydro-[1,2,4]triazolo[4,3-f]pteridine (Example 113),
4-perdeuteroethyl-5-perdeuteroisopropyl-1-methyl-7-(2-(3-(trifluoromethoxy)phenyl)-1H-imidazol-1-yl)-4,5-dihydro-[1,2,4]triazolo[4,3-f]pteridine (Example 114),
4-perdeuteroethyl-5-perdeuteroisopropyl-7-(2-(4-(trifluoromethyl)phenyl)-1H-imidazol-1-yl)-4,5-dihydro-[1,2,4]triazolo[4,3-f]pteridine (Example 115),
4-perdeuteroethyl-5-perdeuteroisopropyl-1-methyl-7-(2-(4-(trifluoromethyl)phenyl)-1H-imidazol-1-yl)-4,5-dihydro-[1,2,4]triazolo[4,3-f]pteridine (Example 116),
(R)-4-perdeuteroethyl-7-(2-(4-fluorophenyl)-1H-imidazol-1-yl)-5-perdeuteroisopropyl-4,5-dihydro-[1,2,4]triazolo[4,3-f]pteridine and (S)-4-perdeuteroethyl-7-(2-(4-fluorophenyl)-1H-imidazol-1-yl)-5-perdeuteroisopropyl-4,5-dihydro-[1,2,4]triazolo[4,3-f]pteridine (Example 117),
(R)-4-perdeuteroethyl-7-(2-(4-fluorophenyl)-1H-imidazol-1-yl)-5-perdeuteroisopropyl-1-methyl-4,5-dihydro-[1,2,4]triazolo[4,3-f]pteridine and (S)-4-perdeuteroethyl-7-(2-(4-fluorophenyl)-1H-imidazol-1-yl)-5-perdeuteroisopropyl-1-methyl-4,5-dihydro-[1,2,4]triazolo[4,3-f]pteridine (Example 118),
(R)-7-(2-(3,5-difluorophenyl)-1H-imidazol-1-yl)-4-perdeuteroethyl-5-perdeuteroisopropyl-4,5-dihydro-[1,2,4]triazolo[4,3-f]pteridine and (S)-7-(2-(3,5-difluorophenyl)-1H-imidazol-1-yl)-4-perdeuteroethyl-5-perdeuteroisopropyl-4,5-dihydro-[1,2,4]triazolo[4,3-f]pteridine (Example 119),
(R)-7-(2-(3,5-difluorophenyl)-1H-imidazol-1-yl)-4-perdeuteroethyl-5-perdeuteroisopropyl-1-methyl-4,5-dihydro-[1,2,4]triazolo[4,3-f]pteridine and (S)-7-(2-(3,5-difluorophenyl)-1H-imidazol-1-yl)-4-perdeuteroethyl-5-perdeuteroisopropyl-1-methyl-4,5-dihydro-[1,2,4]triazolo[4,3-f]pteridine (Example 120),
7-(2-(3,4-difluorophenyl)-1H-imidazol-1-yl)-4-perdeuteroethyl-5-perdeuteroisopropyl-4,5-dihydro-[1,2,4]triazolo[4,3-f]pteridine (Example 121),
7-(2-(3,4-difluorophenyl)-1H-imidazol-1-yl)-4-perdeuteroethyl-5-perdeuteroisopropyl-1-methyl-4,5-dihydro-[1,2,4]triazolo[4,3-f]pteridine (Example 122),
4-perdeuteroethyl-5-perdeuteroisopropyl-7-(2-(4-(trifluoromethoxy)phenyl)-1H-imidazol-1-yl)-4,5-dihydro-[1,2,4]triazolo[4,3-f]pteridine (Example 123),
4-perdeuteroethyl-5-perdeuteroisopropyl-1-methyl-7-(2-(4-(trifluoromethoxy)phenyl)-1H-imidazol-1-yl)-4,5-dihydro-[1,2,4]triazolo[4,3-f]pteridine (Example 124),
4-perdeuteroethyl-5-perdeuteroisopropyl-7-(2-(3-(trifluoromethyl)phenyl)-1H-imidazol-1-yl)-4,5-dihydro-[1,2,4]triazolo[4,3-f]pteridine (Example 125),
4-perdeuteroethyl-5-perdeuteroisopropyl-1-methyl-7-(2-(3-(trifluoromethyl)phenyl)-1H-imidazol-1-yl)-4,5-dihydro-[1,2,4]triazolo[4,3-f]pteridine (Example 126),
(R)-4-perdeuteroethyl-7-(2-(3-fluorophenyl)-1H-imidazol-1-yl)-5-perdeuteroisopropyl-4,5-dihydro-[1,2,4]triazolo[4,3-f]pteridine and (S)-4-perdeuteroethyl-7-(2-(3-fluorophenyl)-1H-imidazol-1-yl)-5-perdeuteroisopropyl-4,5-dihydro-[1,2,4]triazolo[4,3-f]pteridine (Example 127),
(R)-4-perdeuteroethyl-7-(2-(3-fluorophenyl)-1H-imidazol-1-yl)-5-perdeuteroisopropyl-1-methyl-4,5-dihydro-[1,2,4]triazolo[4,3-f]pteridine and (S)-4-perdeuteroethyl-7-(2-(3-fluorophenyl)-1H-imidazol-1-yl)-5-perdeuteroisopropyl-1-methyl-4,5-dihydro-[1,2,4]triazolo[4,3-f]pteridine (Example 128),
(4R)-4-ethyl-7-(2-phenyl-1H-imidazol-1-yl)-5-(tetrahydrofuran-3-yl)-4,5-dihydro-[1,2,4]triazolo[4,3-f]pteridine (Example 129),
(4R)-4-ethyl-1-methyl-7-(2-phenyl-1-imidazol-1-yl)-5-(tetrahydrofuran-3-yl)-4,5-dihydro-[1,2,4]triazolo[4,3-f]pteridine (Example 130),
(S)-7-(2-(3,5-dichlorophenyl)-1H-imidazol-1-yl)-12a-ethyl-10,11,12,12a-tetrahydropyrrolo[2,1-h][1,2,4]triazolo[4,3-f]pteridine (Example 131),
(S)-7-(2-(3,5-dichlorophenyl)-1H-imidazol-1-yl)-12a-ethyl-3-methyl-10,11,12,12a-tetrahydropyrrolo[2,1-h][1,2,4]triazolo[4,3-f]pteridine (Example 132),
(R)-4-ethyl-7-(2-phenyl-1H-imidazol-1-yl)-5-(3,3,3-trifluoropropyl)-4,5-dihydro-[1,2,4]triazolo[4,3-f]pteridine (Example 133),
(R)-4-ethyl-1-methyl-7-(2-phenyl-1H-imidazol-1-yl)-5-(3,3,3-trifluoropropyl)-4,5-dihydro-[1,2,4]triazolo[4,3-f]pteridine (Example 134),
(4R)-7-(2-(3,4-difluorophenyl)-1H-imidazol-1-yl)-4-ethyl-5-(tetrahydrofuran-3-yl)-4,5-dihydro-[1,2,4]triazolo[4,3-f]pteridine (Example 135),
(4R)-7-(2-(3,4-difluorophenyl)-1H-imidazol-1-yl)-4-ethyl-1-methyl-5-(tetrahydrofuran-3-yl)-4,5-dihydro-[1,2,4]triazolo[4,3-f]pteridine (Example 136),
(4R)-4-ethyl-7-(2-(3-fluorophenyl)-1H-imidazol-1-yl)-5-(tetrahydrofuran-3-yl)-4,5-dihydro-[1,2,4]triazolo[4,3-f]pteridine (Example 137),
(4R)-4-ethyl-7-(2-(3-fluorophenyl)-1H-imidazol-1-yl)-1-methyl-5-(tetrahydrofuran-3-yl)-4,5-dihydro-[1,2,4]triazolo[4,3-f]pteridine (Example 138),
(4R)-4-ethyl-7-(2-phenyl-1H-imidazol-1-yl)-5-(tetrahydrofuran-3-yl)-4,5-dihydro-[1,2,4]triazolo[4,3-f]pteridine (Example 139),
(4R)-4-ethyl-1-methyl-7-(2-phenyl-1H-imidazol-1-yl)-5-(tetrahydrofuran-3-yl)-4,5-dihydro-[1,2,4]triazolo[4,3-f]pteridine (Example 140),
(S)-12a-ethyl-7-(2-(isoquinolin-1-yl)-1H-imidazol-1-yl)-10,11,12,12a-tetrahydropyrrolo[2,1-h][1,2,4]triazolo[4,3-f]pteridine (Example 141),
(S)-12a-ethyl-7-(2-(isoquinolin-1-yl)-1H-imidazol-1-yl)-3-methyl-10,11,12,12a-tetrahydropyrrolo[2,1-h][1,2,4]triazolo[4,3-f]pteridine (Example 142),
(4R)-7-(2-(3-chlorophenyl)-1H-imidazol-1-yl)-4-ethyl-5-(tetrahydrofuran-3-yl)-4,5-dihydro-[1,2,4]triazolo[4,3-f]pteridine (Example 143),
(4R)-7-(2-(3-chlorophenyl)-1H-imidazol-1-yl)-4-ethyl-1-methyl-5-(tetrahydrofuran-3-yl)-4,5-dihydro-[1,2,4]triazolo[4,3-f]pteridine (Example 144),
(S)-7-(2-(3-chloro-4-fluorophenyl)-1H-imidazol-1-yl)-12a-ethyl-10,11,12,12a-tetrahydropyrrolo[2,1-h][1,2,4]triazolo[4,3-f]pteridine (Example 145),
(S)-7-(2-(3-chloro-4-fluorophenyl)-1H-imidazol-1-yl)-12a-ethyl-3-methyl-10,11,12,12a-tetrahydropyrrolo[2,1-h][1,2,4]triazolo[4,3-f]pteridine (Example 146),
(S)-7-(2-(5-chlorothiophen-2-yl)-1H-imidazol-1-yl)-12a-ethyl-10,11,12,12a-tetrahydropyrrolo[2,1-h][1,2,4]triazolo[4,3-f]pteridine (Example 147),
(S)-7-(2-(5-chlorothiophen-2-yl)-1H-imidazol-1-yl)-12a-ethyl-3-methyl-10,11,12,12a-tetrahydropyrrolo[2,1-h][1,2,4]triazolo[4,3-f]pteridine (Example 148),
2-(1-((4R)-4-ethyl-5-(tetrahydrofuran-3-yl)-4,5-dihydro-[1,2,4]triazolo[4,3-f]pteridin-7-yl)-1H-imidazol-2-yl)thiazole (Example 149),
2-(1-((4R)-4-ethyl-1-methyl-5-(tetrahydrofuran-3-yl)-4,5-dihydro-[1,2,4]triazolo[4,3-f]pteridin-7-yl)-1H-imidazol-2-yl)thiazole (Example 150),
(S)-12a-ethyl-7-(2-(3-fluorophenyl)-1H-imidazol-1-yl)-10,11,12,12a-tetrahydropyrrolo[2,1-h][1,2,4]triazolo[4,3-f]pteridine (Example 151),
(S)-12a-ethyl-7-(2-(3-fluorophenyl)-1H-imidazol-1-yl)-3-methyl-10,11,12,12a-tetrahydropyrrolo[2,1-h][1,2,4]triazolo[4,3-f]pteridine (Example 152),
(S)-7-(2-(3,5-difluorophenyl)-1H-imidazol-1-yl)-12a-ethyl-10,11,12,12a-tetrahydropyrrolo[2,1-h][1,2,4]triazolo[4,3-f]pteridine (Example 153),
(S)-7-(2-(3,5-difluorophenyl)-1H-imidazol-1-yl)-12a-ethyl-3-methyl-10,11,12,12a-tetrahydropyrrolo[2,1-h][1,2,4]triazolo[4,3-f]pteridine (Example 154),
(4R)-4-ethyl-7-(2-(4-fluorophenyl)-1H-imidazol-1-yl)-5-(tetrahydrofuran-3-yl)-4,5-dihydro-[1,2,4]triazolo[4,3-f]pteridine (Example 155),
(4R)-4-ethyl-7-(2-(4-fluorophenyl)-1H-imidazol-1-yl)-1-methyl-5-(tetrahydrofuran-3-yl)-4,5-dihydro-[1,2,4]triazolo[4,3-f]pteridine (Example 156),
(4R)-7-(2-(3-chloro-4-fluorophenyl)-1H-imidazol-1-yl)-4-ethyl-5-(tetrahydrofuran-3-yl)-4,5-dihydro-[1,2,4]triazolo[4,3-f]pteridine (Example 157),
(4R)-7-(2-(3-chloro-4-fluorophenyl)-1H-imidazol-1-yl)-4-ethyl-1-methyl-5-(tetrahydrofuran-3-yl)-4,5-dihydro-[1,2,4]triazolo[4,3-f]pteridine (Example 158),
(R)-4-ethyl-7-(2-(4-fluorophenyl)-1H-imidazol-1-yl)-5-(3,3,3-trifluoropropyl)-4,5-dihydro-[1,2,4]triazolo[4,3-f]pteridine (Example 159),
(R)-4-ethyl-7-(2-(4-fluorophenyl)-1H-imidazol-1-yl)-1-methyl-5-(3,3,3-trifluoropropyl)-4,5-dihydro-[1,2,4]triazolo[4,3-f]pteridine (Example 160),
(R)-4-ethyl-7-(2-(pyridin-4-yl)-1H-imidazol-1-yl)-5-(3,3,3-trifluoropropyl)-4,5-dihydro-[1,2,4]triazolo[4,3-f]pteridine (Example 161),
(R)-4-ethyl-1-methyl-7-(2-(pyridin-4-yl)-1H-imidazol-1-yl)-5-(3,3,3-trifluoropropyl)-4,5-dihydro-[1,2,4]triazolo[4,3-f]pteridine (Example 162),
(S)-7-(2-(3-chlorophenyl)-1H-imidazol-1-yl)-12a-ethyl-10,11,12,12a-tetrahydropyrrolo[2,1-h][1,2,4]triazolo[4,3-f]pteridine (Example 163),
(S)-7-(2-(3-chlorophenyl)-1H-imidazol-1-yl)-12a-ethyl-3-methyl-10,11,12,12a-tetrahydropyrrolo[2,1-h][1,2,4]triazolo[4,3-f]pteridine (Example 164),
(S)-12a-ethyl-7-(2-(quinolin-3-yl)-1H-imidazol-1-yl)-10,11,12,12a-tetrahydropyrrolo[2,1-h][1,2,4]triazolo[4,3-f]pteridine (Example 165),
(S)-12a-ethyl-3-methyl-7-(2-(quinolin-3-yl)-1H-imidazol-1-yl)-10,11,12,12a-tetrahydropyrrolo[2,1-h][1,2,4]triazolo[4,3-f]pteridine (Example 166),
(R)-4-ethyl-7-(2-(5-fluoropyridin-2-yl)-1H-imidazol-1-yl)-5-(3,3,3-trifluoropropyl)-4,5-dihydro-[1,2,4]triazolo[4,3-f]pteridine (Example 167),
(R)-4-ethyl-7-(2-(5-fluoropyridin-2-yl)-1H-imidazol-1-yl)-1-methyl-5-(3,3,3-trifluoropropyl)-4,5-dihydro-[1,2,4]triazolo[4,3-f]pteridine (Example 168),
(S)-12a-ethyl-7-(2-(3-(trifluoromethoxy)phenyl)-1H-imidazol-1-yl)-10,11,12,12a-tetrahydropyrrolo[2,1-h][1,2,4]triazolo[4,3-f]pteridine (Example 169),
(S)-12a-ethyl-3-methyl-7-(2-(3-(trifluoromethoxy)phenyl)-1H-imidazol-1-yl)-10,11,12,12a-tetrahydropyrrolo[2,1-h][1,2,4]triazolo[4,3-f]pteridine (Example 170),
(S)-7-(2-(3-bromophenyl)-1H-imidazol-1-yl)-12a-ethyl-10,11,12,12a-tetrahydropyrrolo[2,1-h][1,2,4]triazolo[4,3-f]pteridine (Example 171),
(S)-7-(2-(3-bromophenyl)-1H-imidazol-1-yl)-12a-ethyl-3-methyl-10,11,12,12a-tetrahydropyrrolo[2,1-h][1,2,4]triazolo[4,3-f]pteridine (Example 172),
(4R)-7-(2-(2,3-difluorophenyl)-1H-imidazol-1-yl)-4-ethyl-5-(tetrahydrofuran-3-yl)-4,5-dihydro-[1,2,4]triazolo[4,3-f]pteridine (Example 173),
(4R)-7-(2-(2,3-difluorophenyl)-1H-imidazol-1-yl)-4-ethyl-1-methyl-5-(tetrahydrofuran-3-yl)-4,5-dihydro-[1,2,4]triazolo[4,3-f]pteridine (Example 174),
(R)-7-(2-(2,3-difluorophenyl)-1H-imidazol-1-yl)-4-ethyl-5-(3,3,3-trifluoropropyl)-4,5-dihydro-[1,2,4]triazolo[4,3-f]pteridine (Example 175),
(R)-7-(2-(2,3-difluorophenyl)-1H-imidazol-1-yl)-4-ethyl-1-methyl-5-(3,3,3-trifluoropropyl)-4,5-dihydro-[1,2,4]triazolo[4,3-f]pteridine (Example 176),
7-(2-(4-fluorophenyl)-1H-imidazol-1-yl)-4-(2,2,2-trifluoroethyl)-5-(3,3,3-trifluoropropyl)-4,5-dihydro-[1,2,4]triazolo[4,3-f]pteridine (Example 177),
7-(2-(4-fluorophenyl)-1H-imidazol-1-yl)-1-methyl-4-(2,2,2-trifluoroethyl)-5-(3,3,3-trifluoropropyl)-4,5-dihydro-[1,2,4]triazolo[4,3-f]pteridine (Example 178),
(4R)-7-(2-(2,4-difluorophenyl)-1H-imidazol-1-yl)-4-ethyl-5-(tetrahydrofuran-3-yl)-4,5-dihydro-[1,2,4]triazolo[4,3-f]pteridine (Example 179),
(4R)-7-(2-(2,4-difluorophenyl)-1H-imidazol-1-yl)-4-ethyl-1-methyl-5-(tetrahydrofuran-3-yl)-4,5-dihydro-[1,2,4]triazolo[4,3-f]pteridine (Example 180),
(R)-7-(2-(2,4-difluorophenyl)-1H-imidazol-1-yl)-4-ethyl-5-(3,3,3-trifluoropropyl)-4,5-dihydro-[1,2,4]triazolo[4,3-f]pteridine (Example 181),
(R)-7-(2-(2,4-difluorophenyl)-1H-imidazol-1-yl)-4-ethyl-1-methyl-5-(3,3,3-trifluoropropyl)-4,5-dihydro-[1,2,4]triazolo[4,3-f]pteridine (Example 182),
(R)-7-(2-(2-chloro-4-fluorophenyl)-1H-imidazol-1-yl)-4-ethyl-5-(3,3,3-trifluoropropyl)-4,5-dihydro-[1,2,4]triazolo[4,3-f]pteridine (Example 183),
(R)-7-(2-(2-chloro-4-fluorophenyl)-1H-imidazol-1-yl)-4-ethyl-1-methyl-5-(3,3,3-trifluoropropyl)-4,5-dihydro-[1,2,4]triazolo[4,3-f]pteridine (Example 184),
(R)-13a-ethyl-7-(2-phenyl-1H-imidazol-1-yl)-10,11,13,13a-tetrahydro-[1,4]oxazino[3,4-h][1,2,4]triazolo[4,3-f]pteridine and (S)-13a-ethyl-7-(2-phenyl-1H-imidazol-1-yl)-10,11,13,13a-tetrahydro-[1,4]oxazino[3,4-h][1,2,4]triazolo[4,3-f]pteridine (Example 185),
(R)-13a-ethyl-3-methyl-7-(2-phenyl-1H-imidazol-1-yl)-10,11,13,13a-tetrahydro-[1,4]oxazino[3,4-h][1,2,4]triazolo[4,3-f]pteridine and (S)-13a-ethyl-3-methyl-7-(2-phenyl-1H-imidazol-1-yl)-10,11,13,13a-tetrahydro-[1,4]oxazino[3,4-h][1,2,4]triazolo[4,3-f]pteridine (Example 186),
4-ethyl-7-(2-(4-fluorophenyl)-1H-imidazol-1-yl)-5-(1H-pyrazol-3-yl)-4,5-dihydro-[1,2,4]triazolo[4,3-f]pteridine (Example 187), 4-ethyl-7-(2-(4-fluorophenyl)-1H-imidazol-1-yl)-1-methyl-5-(1H-pyrazol-3-yl)-4,5-dihydro-[1,2,4]triazolo[4,3-f]pteridine (Example 188),
(R)-7-(2-(2,4-difluorophenyl)-1H-imidazol-1-yl)-13a-ethyl-10,11,13,13a-tetrahydro-[1,4]oxazino[3,4-h][1,2,4]triazolo[4,3-f]pteridine and (S)-7-(2-(2,4-difluorophenyl)-1H-imidazol-1-yl)-13a-ethyl-10,11,13,13a-tetrahydro-[1,4]oxazino[3,4-h][1,2,4]triazolo[4,3-f]pteridine (Example 189),
(R)-7-(2-(2,4-difluorophenyl)-1H-imidazol-1-yl)-13a-ethyl-3-methyl-10,11,13,13a-tetrahydro-[1,4]oxazino[3,4-h][1,2,4]triazolo[4,3-f]pteridine and (S)-7-(2-(2,4-difluorophenyl)-1H-imidazol-1-yl)-13a-ethyl-3-methyl-10,11,13,13a-tetrahydro-[1,4]oxazino[3,4-h][1,2,4]triazolo[4,3-f]pteridine (Example 190),
(R)-13a-ethyl-7-(2-(5-fluoropyridin-2-yl)-1H-imidazol-1-yl)-10,11,13,13a-tetrahydro-[1,4]oxazino[3,4-h][1,2,4]triazolo[4,3-f]pteridine and (S)-13a-ethyl-7-(2-(5-fluoropyridin-2-yl)-1H-imidazol-1-yl)-10,11,13,13a-tetrahydro-[1,4]oxazino[3,4-h][1,2,4]triazolo[4,3-f]pteridine (Example 191),
(R)-13a-ethyl-7-(2-(5-fluoropyridin-2-yl)-1H-imidazol-1-yl)-3-methyl-10,11,13,13a-tetrahydro-[1,4]oxazino[3,4-h][1,2,4]triazolo[4,3-f]pteridine and (S)-13a-ethyl-7-(2-(5-fluoropyridin-2-yl)-1H-imidazol-1-yl)-3-methyl-10,11,13,13a-tetrahydro-[1,4]oxazino[3,4-h][1,2,4]triazolo[4,3-f]pteridine (Example 192),
(R)-13a-ethyl-7-(2-(thiazol-2-yl)-1H-imidazol-1-yl)-10,11,13,13a-tetrahydro-[1,4]oxazino[3,4-h][1,2,4]triazolo[4,3-f]pteridine and (S)-13a-ethyl-7-(2-(thiazol-2-yl)-1H-imidazol-1-yl)-10,11,13,13a-tetrahydro-[1,4]oxazino[3,4-h][1,2,4]triazolo[4,3-f]pteridine (Example 193),
(R)-13a-ethyl-3-methyl-7-(2-(thiazol-2-yl)-1H-imidazol-1-yl)-10,11,13,13a-tetrahydro-[1,4]oxazino[3,4-h][1,2,4]triazolo[4,3-f]pteridine and (S)-13a-ethyl-3-methyl-7-(2-(thiazol-2-yl)-1H-imidazol-1-yl)-10,11,13,13a-tetrahydro-[1,4]oxazin[3,4-h][1,2,4]triazolo[4,3-f]pteridine (Example 194),
7-(2-(2,3-difluorophenyl)-1H-imidazol-1-yl)-13a-ethyl-10,11,13,13a-tetrahydro-[1,4]oxazino[3,4-h][1,2,4]triazolo[4,3-f]pteridine (Example 751), and
7-(2-(2,3-difluorophenyl)-1H-imidazol-1-yl)-13a-ethyl-3-methyl-10,11,13,13a-tetrahydro-[1,4]oxazino[3,4-h][1,2,4]triazolo[4,3-f]pteridine (Example 752).
   For Examples 187 and 188, the SEM nitrogen protecting group is removed similarly to the method of Example 655. The following table provides the example number (column 1), Intermediate used (column 2), ring reactant used (column 3), and trimethyl orthoformate (F) or trimethyl orthoacetate (Ac) in the final step (column 4) and to give the compound shown in column 5.

| | | | Formate | |
|---|---|---|---|---|
| Ex. | | | or | |
| No. | Int. | Ring reactant | Acetate | Compound structure |
| 14 | E-0 | 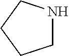 | Ac | 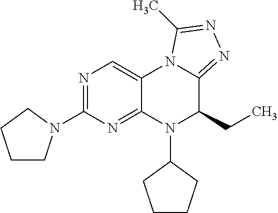 |
| 15 | E-0 | 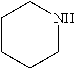 | F | 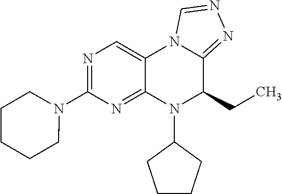 |
| 16 | | | Ac | 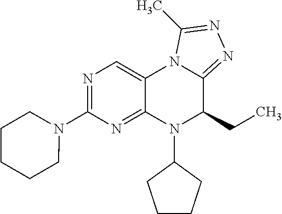 |
| 17 | E-0 | 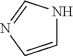 | F | 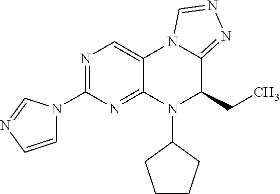 |
| 18 | | | Ac | 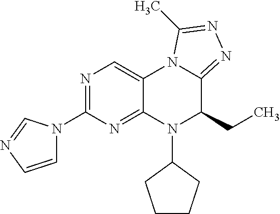 |
| 19 | E-0 | 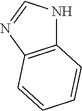 | F | 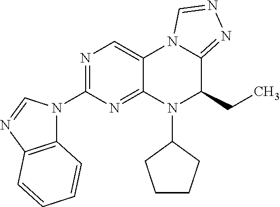 |
| 20 | | | Ac | 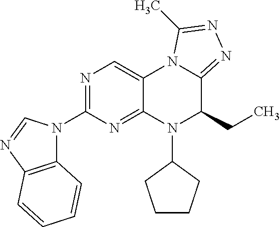 |
| 45 | E-0 | 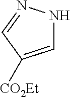 | F | 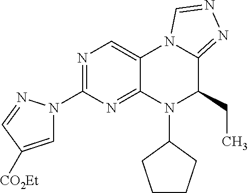 |
| 46 | | | Ac | 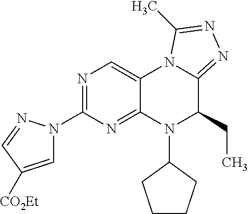 |
| 53 | G-1 | 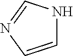 | F | 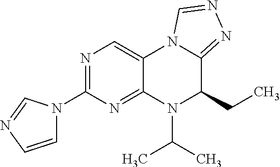 |
| 54 | | | Ac | 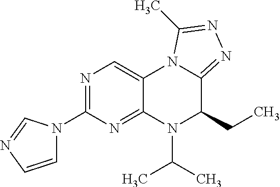 |
| 57 | I-1 | 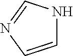 | F | 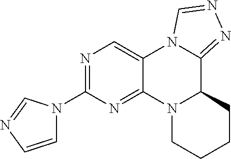 |
| 58 | | | Ac | 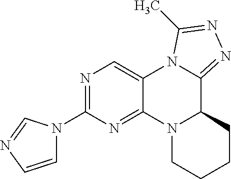 |
| 67 | E-0 | 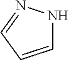 | F | 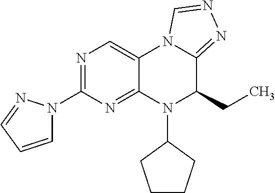 |
| 68 | | | Ac | 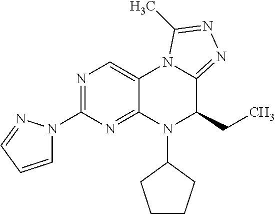 |
| 73 | K-1 | 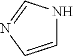 | F | 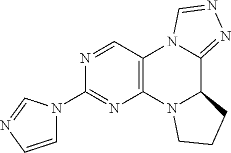 |
| 74 | | | Ac | 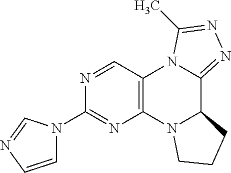 |
| 99 | XX-1 | 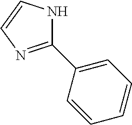 | F | 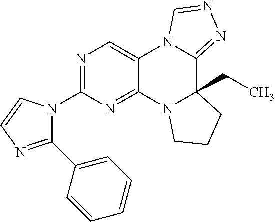 |
| 100 | | | Ac | 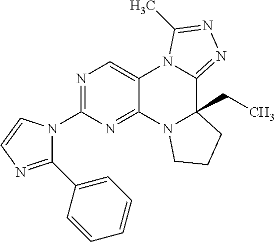 |
| 101 | T-1 | 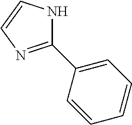 | F | 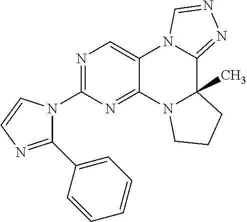 |
| 102 | | | Ac | 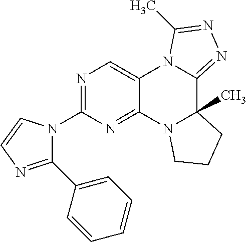 |
| 103 | E-0 | 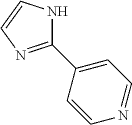 | F | 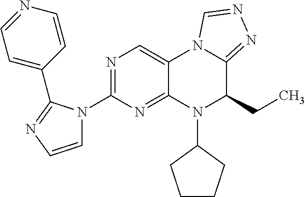 |
| 104 | | | Ac | 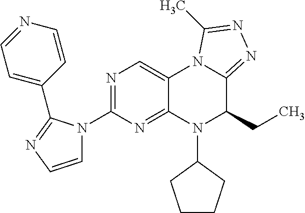 |
| 105 | XX-1 | 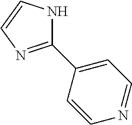 | F | 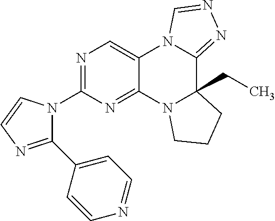 |
| 106 | | | Ac | 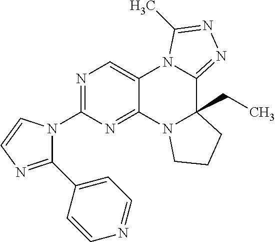 |
| 107 | XX-1 | 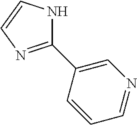 | F | 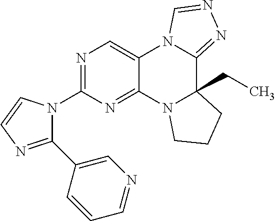 |
| 108 | | | Ac | 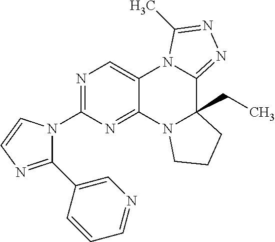 |
| 109 | XX-1 | 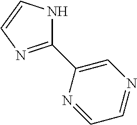 | F | 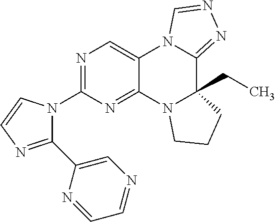 |
| 110 | | | Ac | 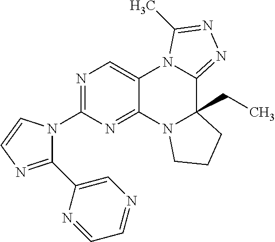 |
| 111 | XX-1 | 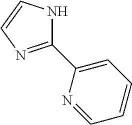 | F | 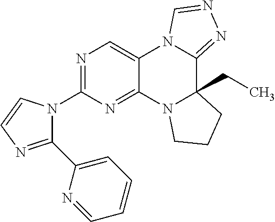 |
| 112 | | | Ac | 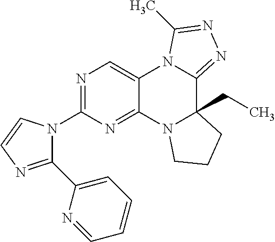 |
| 113 | Q-1 | 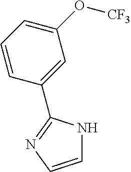 | F | 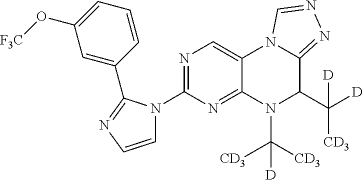 |
| 114 | | | Ac | 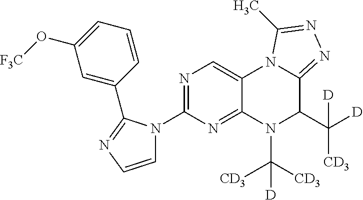 |
| 115 | Q-1 | 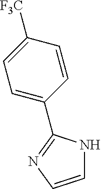 | F | 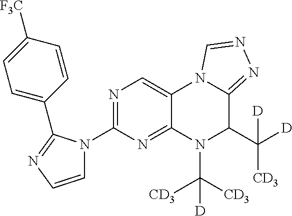 |
| 116 | | | Ac | 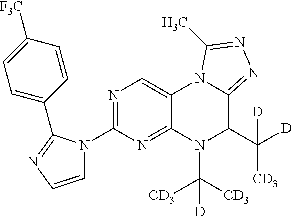 |
| 117(R) | Q-1 | 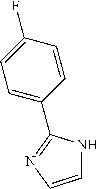 | F | 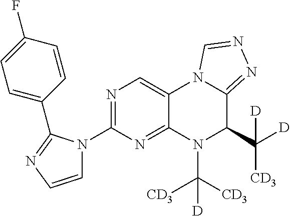 |
| 117(S) | | | | 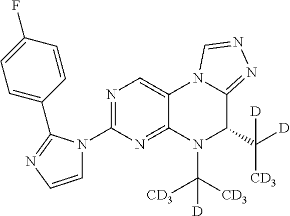 |
| 118(R) | Q-1 | 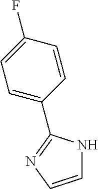 | Ac | 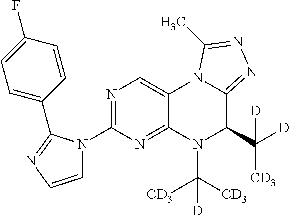 |
| 118(S) | | | | 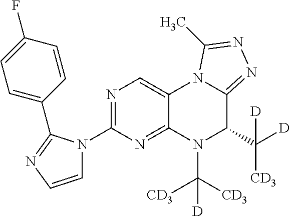 |
| 119(R) | Q-1 | 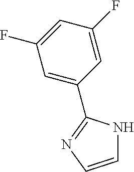 | F | 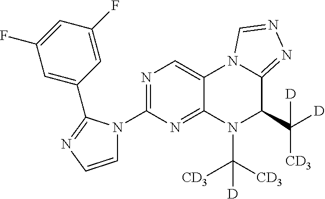 |
| 119(S) | | | | 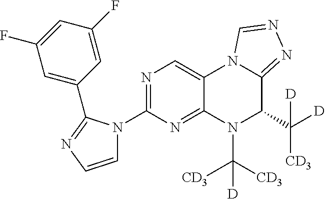 |
| 120(R) | Q-1 | 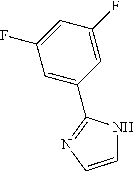 | Ac | 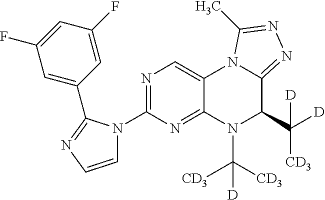 |
| 120(S) | | | | 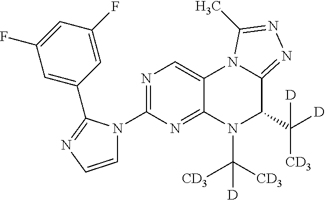 |
| 121 | Q-1 | 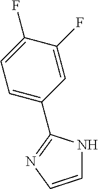 | F | 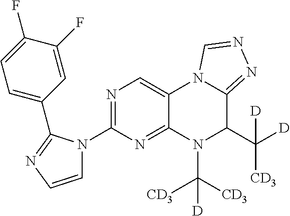 |
| 122 | | | Ac | 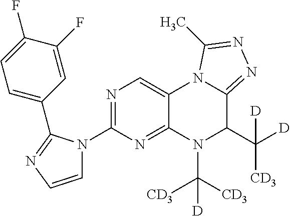 |
| 123 | Q-1 | 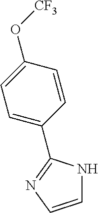 | F | 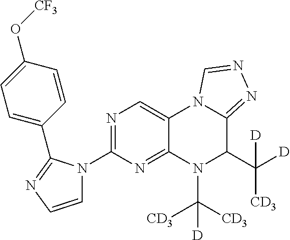 |
| 124 | | | Ac | 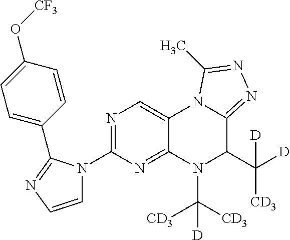 |
| 125 | Q-1 | 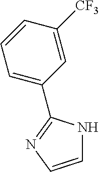 | F | 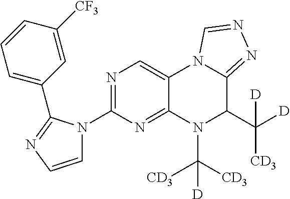 |
| 126 | | | Ac | 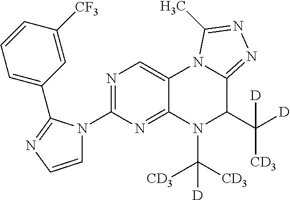 |
| 127(R) | Q-1 | 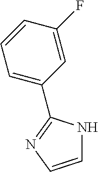 | F | 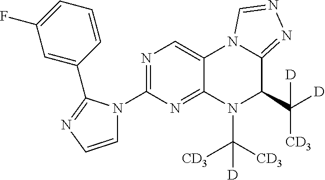 |
| 127(S) | | | | 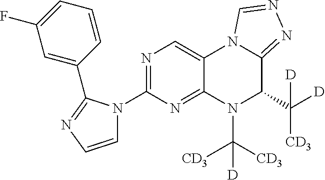 |
| 128(R) | Q-1 | 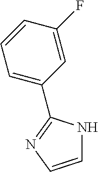 | Ac | 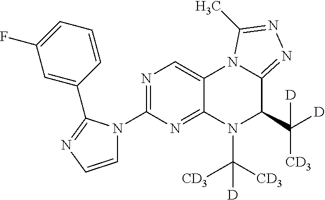 |
| 128(S) | | | | 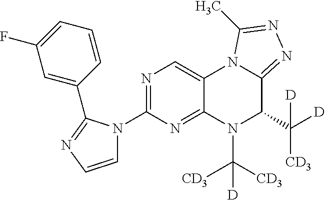 |
| 129 | N-1 | 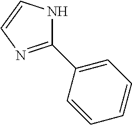 | F | 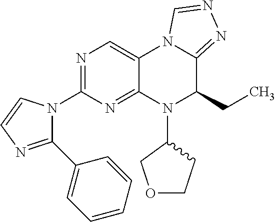 |
| 130 | | | Ac | 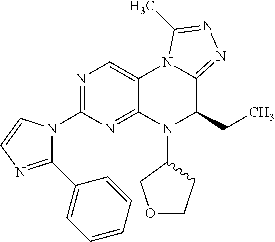 |
| 131 | XX-1 | 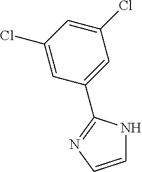 | F | 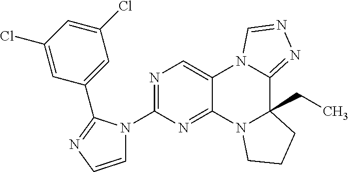 |
| 132 | | | Ac | 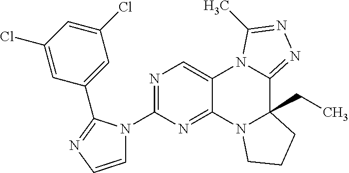 |
| 133 | U-1 | 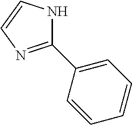 | F | 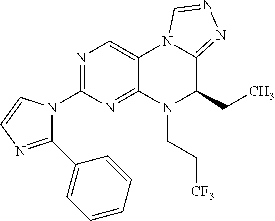 |
| 134 | | | Ac | 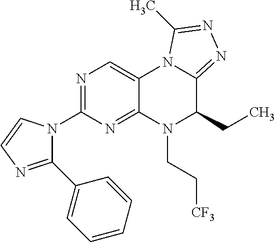 |
| 135 | N-1 | 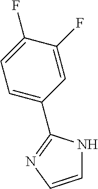 | F | 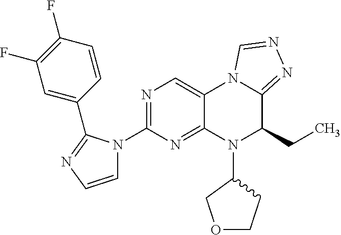 |
| 136 | | | Ac | 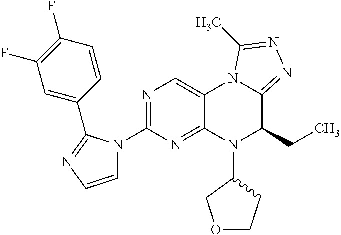 |
| 137 | N-1 | 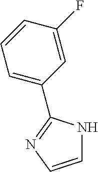 | F | 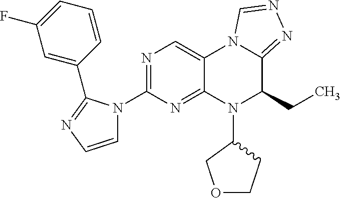 |
| 138 | | | Ac | 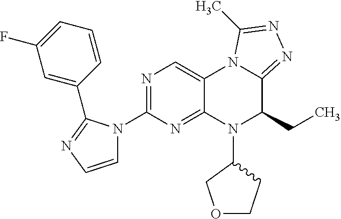 |
| 139 | N-1 | 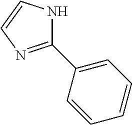 | F | 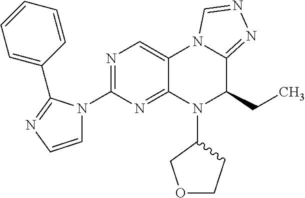 |
| 140 | | | Ac | 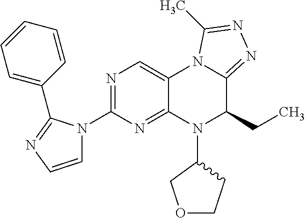 |
| 141 | XX-1 | 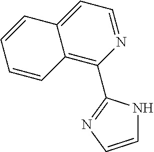 | F | 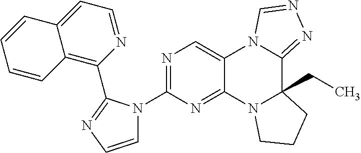 |
| 142 | | | Ac | 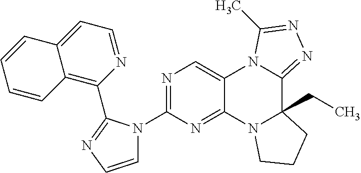 |
| 143 | N-1 | 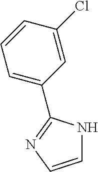 | F | 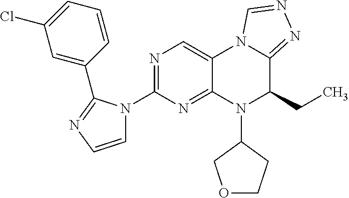 |
| 144 | | | Ac | 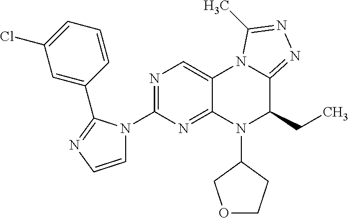 |
| 145 | XX-1 | 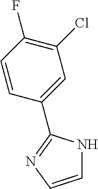 | F | 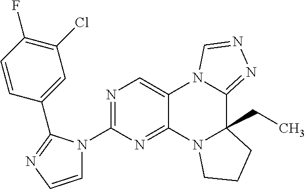 |
| 146 | | | Ac | 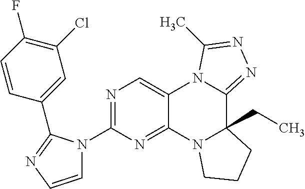 |
| 147 | XX-1 | 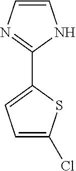 | F | 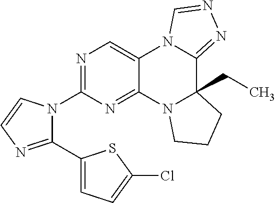 |
| 148 | | | Ac | 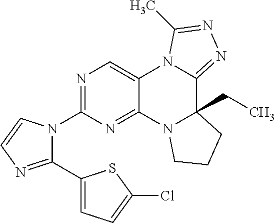 |
| 149 | N-1 | 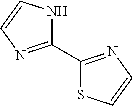 | F | 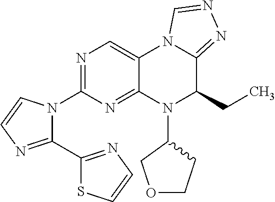 |
| 150 | | | Ac | 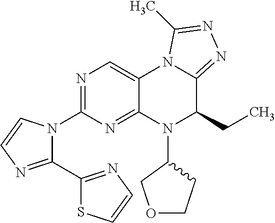 |
| 151 | XX-1 | 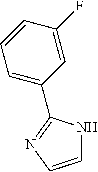 | F | 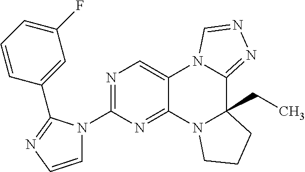 |
| 152 | | | Ac | 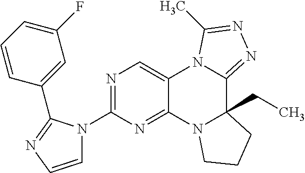 |
| 153 | XX-1 | 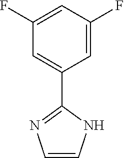 | F | 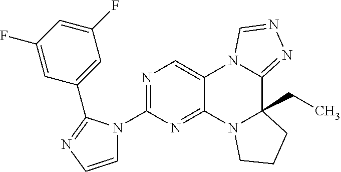 |
| 154 | | | Ac | 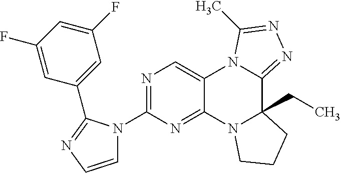 |
| 155 | N-1 | 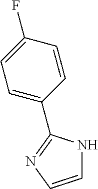 | F | 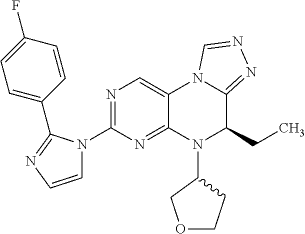 |
| 156 | | | Ac | 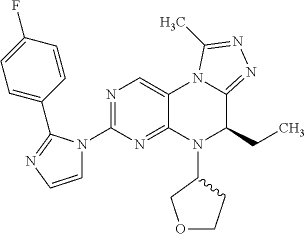 |
| 157 | N-1 | 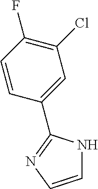 | F | 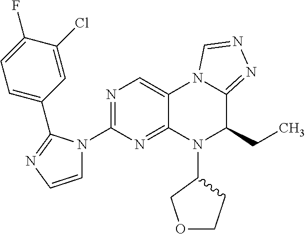 |
| 158 | | | Ac | 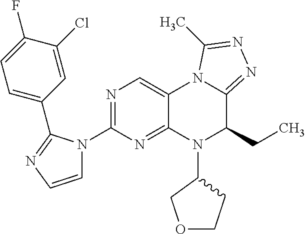 |
| 159 | U-1 | 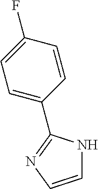 | F | 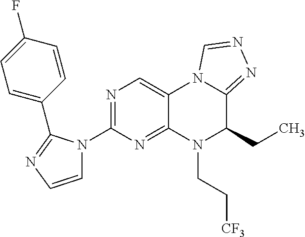 |
| 160 | | | Ac | 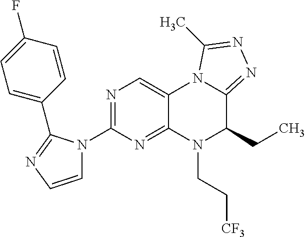 |
| 161 | U-1 | 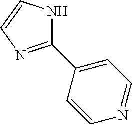 | F | 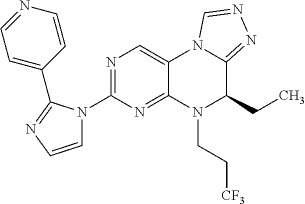 |
| 162 | | | Ac | 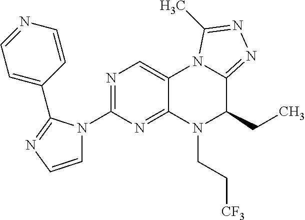 |
| 163 | XX-1 | 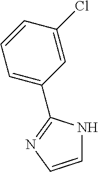 | F | 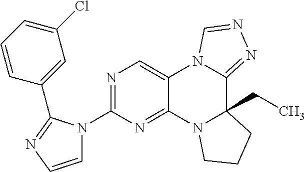 |
| 164 | | | Ac | 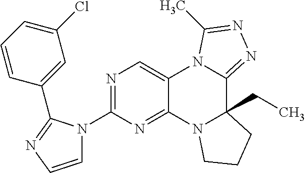 |
| 165 | XX-1 | 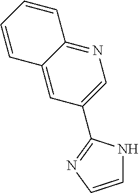 | F | 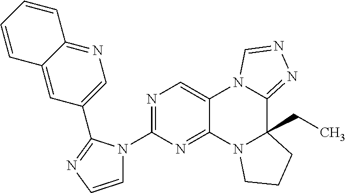 |
| 166 | | | Ac | 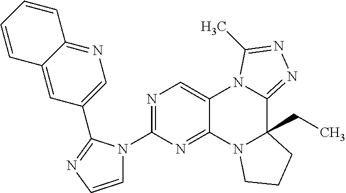 |
| 167 | U-1 | 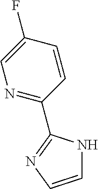 | F | 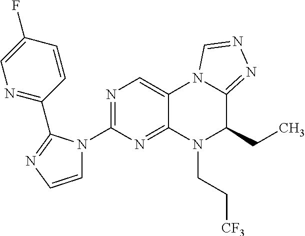 |
| 168 | | | Ac | 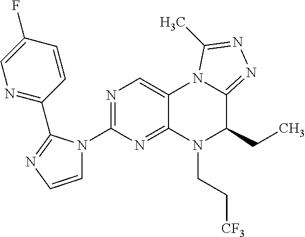 |
| 169 | XX-1 | 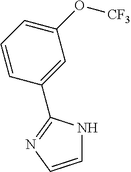 | F | 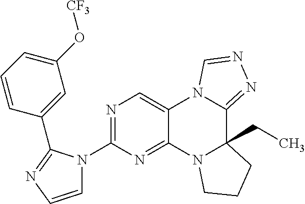 |
| 170 | | | Ac | 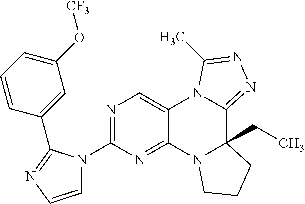 |
| 171 | XX-1 | 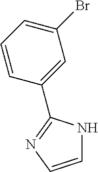 | F | 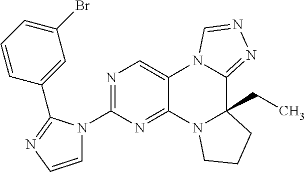 |
| 172 | | | Ac | 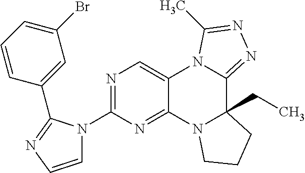 |
| 173 | N-1 | 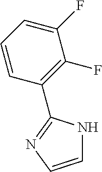 | F | 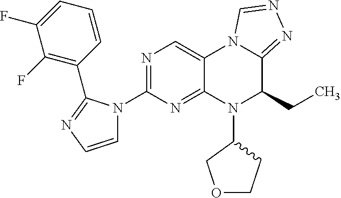 |
| 174 | | | Ac | 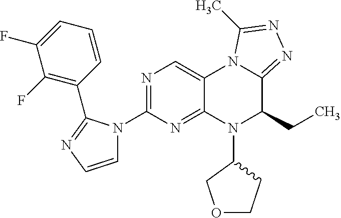 |
| 175 | U-1 | 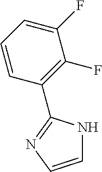 | F | 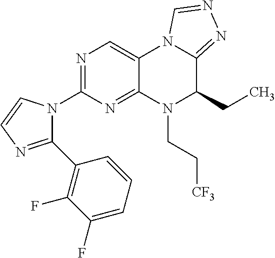 |
| 176 | | | Ac | 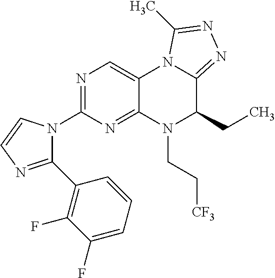 |
| 177 | BB-1 | 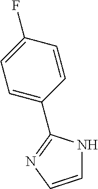 | F | 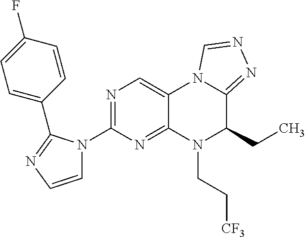 |
| 178 | | | Ac | 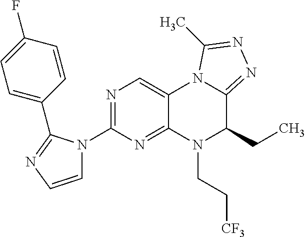 |
| 179 | N-1 | 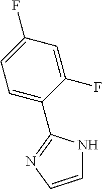 | F | 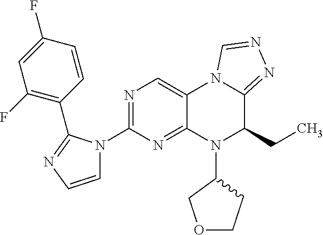 |
| 180 | | | Ac | 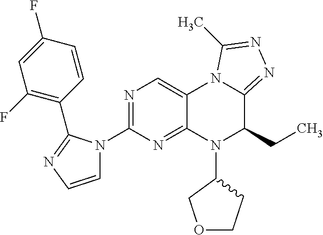 |
| 181 | U-1 | 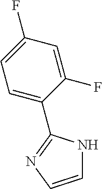 | F | 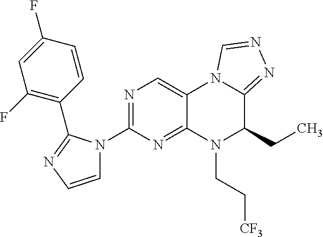 |
| 182 | | | Ac | 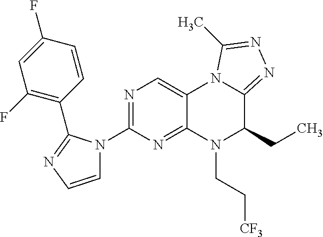 |
| 183 | U-1 | 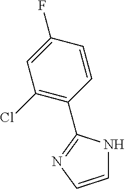 | F | 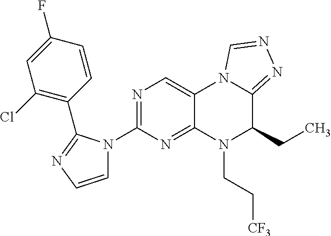 |
| 184 | | | Ac | 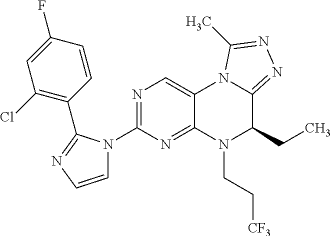 |
| 185(R) | Z-1 | 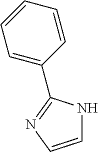 | F | 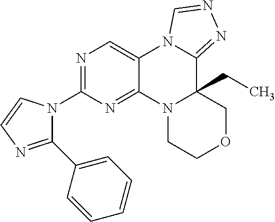 |
| 185(S) | | | | 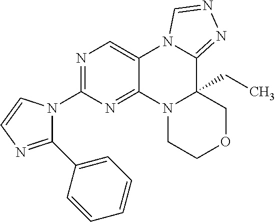 |
| 186(R) | Z-1 | 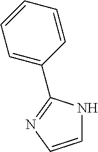 | Ac | 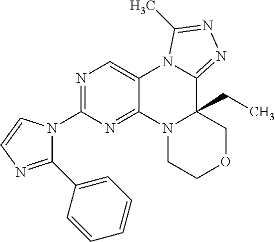 |
| 186(S) | | | | 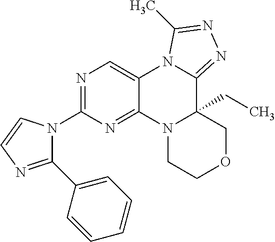 |
| 187 | QQ-1 | 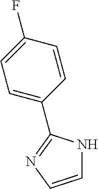 | F | 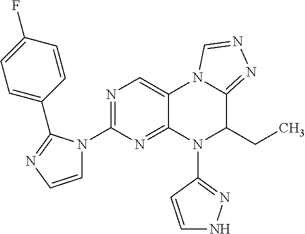 |
| 188 | | | Ac | 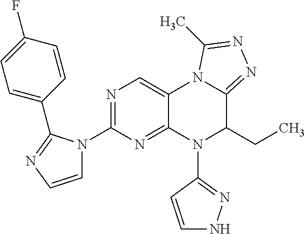 |
| 189(R) | Z-1 | 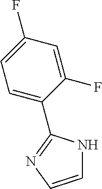 | F | 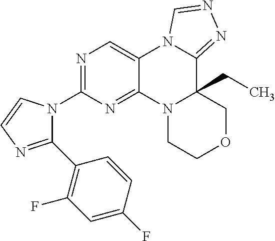 |
| 189(S) | | | | 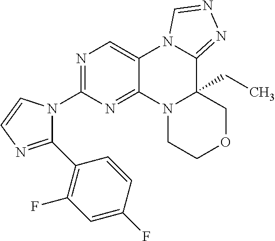 |
| 190(R) | Z-1 | 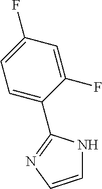 | Ac | 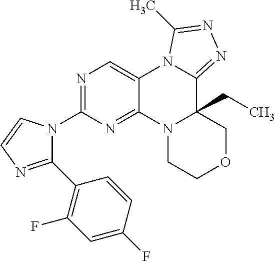 |
| 190(S) | | | | 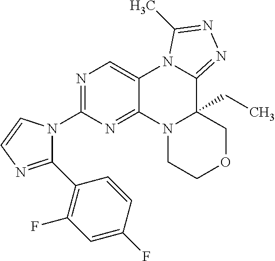 |
| 191(R) | Z-1 | 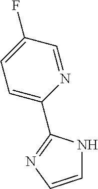 | F | 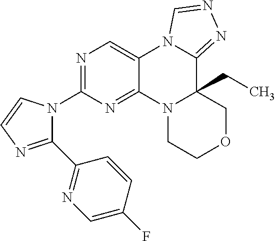 |
| 191(S) | | | | 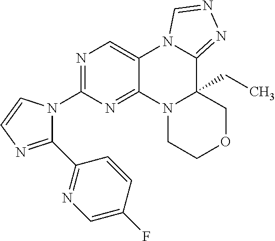 |
| 192(R) | Z-1 | 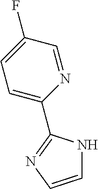 | Ac | 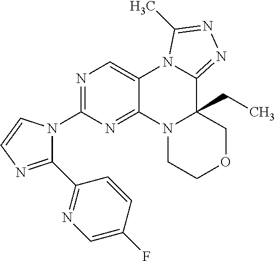 |
| 192(S) | | | | 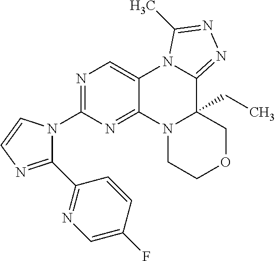 |
| 193(R) | Z-1 | 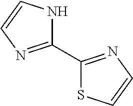 | F | 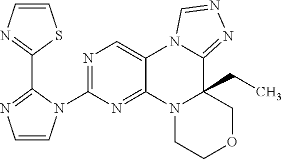 |
| 193(S) | | | | 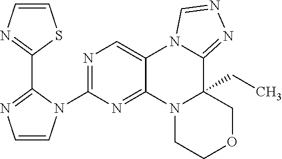 |
| 194(R) | Z-1 | 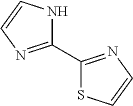 | Ac | 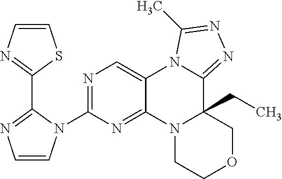 |
| 194(S) | | | | 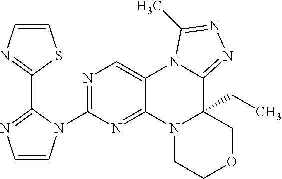 |
| 751 | Z-1 | 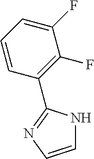 | F | 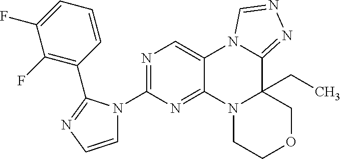 |
| 752 | | | Ac | 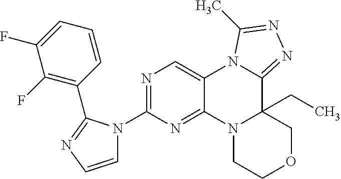 |

### Example 21

### Synthesis of (R)-5-cyclopentyl-4-ethyl-7-(pyridin-4-yl)-4,5-dihydro-[1,2,4]triazolo[4,3-f]pteridine

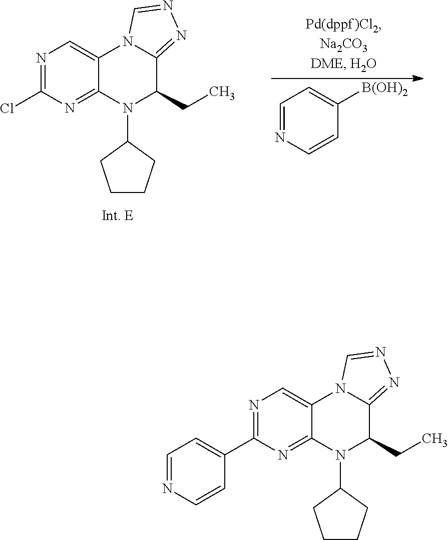

To a solution of Intermediate E in DME and H₂O (4:1) Pd(dppf)Cl₂, Na₂CO₃ and pyridin-4-ylboronic acid are added. The reaction mixture is heated in the microwave at 120° C. for 40 min. The mixture is concentrated and extracted with EtOAc and dried with Na₂SO₄. The solvent is removed and the residue is purified by silica column to give the title compound.

Additional compounds are prepared similarly to this method, optionally replacing Intermediate E with a suitable intermediate, and/or replacing pyridin-4-ylboronic acid with an appropriate boronic acid compound. In some instances, where a racemic mixture results, the two enantiomers may be isolated by chiral chromatography. The following compounds are prepared:
(R)-5-cyclopentyl-4-ethyl-1-methyl-7-(pyridin-4-yl)-4,5-dihydro-[1,2,4]triazolo[4,3-f]pteridine (Example 22),
(R)-5-cyclopentyl-4-ethyl-7-(1H-pyrrol-2-yl)-4,5-dihydro-[1,2,4]triazolo[4,3-f]pteridine (Example 23),
(R)-5-cyclopentyl-4-ethyl-1-methyl-7-(1H-pyrrol-2-yl)-4,5-dihydro-[1,2,4]triazolo[4,3-f]pteridine (Example 24),
(R)-5-cyclopentyl-4-ethyl-7-(1H-pyrazol-4-yl)-4,5-dihydro-[1,2,4]triazolo[4,3-f]pteridine (Example 25),
(R)-5-cyclopentyl-4-ethyl-1-methyl-7-(1H-pyrazol-4-yl)-4,5-dihydro-[1,2,4]triazolo[4,3-f]pteridine (Example 26),
(R)-5-cyclopentyl-4-ethyl-7-(pyridin-2-yl)-4,5-dihydro-[1,2,4]triazolo[4,3-f]pteridine (Example 27),
(R)-5-cyclopentyl-4-ethyl-1-methyl-7-(pyridin-2-yl)-4,5-dihydro-[1,2,4]triazolo[4,3-f]pteridine (Example 28),
(R)-5-cyclopentyl-4-ethyl-7-(1H-indol-2-yl)-4,5-dihydro-[1,2,4]triazolo[4,3-f]pteridine (Example 29),
(R)-5-cyclopentyl-4-ethyl-7-(1H-indol-2-yl)-1-methyl-4,5-dihydro-[1,2,4]triazolo[4,3-f]pteridine (Example 30),
(R)-5-cyclopentyl-4-ethyl-7-(1H-indol-7-yl)-4,5-dihydro-[1,2,4]triazolo[4,3-f]pteridine (Example 31),
(R)-5-cyclopentyl-4-ethyl-7-(1H-indol-7-yl)-1-methyl-4,5-dihydro-[1,2,4]triazolo[4,3-f]pteridine (Example 32),
(R)-5-cyclopentyl-4-ethyl-7-(quinolin-8-yl)-4,5-dihydro-[1,2,4]triazolo[4,3-f]pteridine (Example 33),
(R)-5-cyclopentyl-4-ethyl-1-methyl-7-(quinolin-8-yl)-4,5-dihydro-[1,2,4]triazolo[4,3-f]pteridine (Example 34),
(R)-5-cyclopentyl-4-ethyl-7-phenyl-4,5-dihydro-[1,2,4]triazolo[4,3-f]pteridine (Example 35),
(R)-5-cyclopentyl-4-ethyl-1-methyl-7-phenyl-4,5-dihydro-[1,2,4]triazolo[4,3-f]pteridine (Example 36),
(R)-5-cyclopentyl-4-ethyl-7-(2-fluoropyridin-4-yl)-4,5-dihydro-[1,2,4]triazolo[4,3-f]pteridine (Example 39),
(R)-5-cyclopentyl-4-ethyl-7-(2-fluoropyridin-4-yl)-1-methyl-4,5-dihydro-[1,2,4]triazolo[4,3-f]pteridine (Example 40),
(R)-4-ethyl-5-isopropyl-7-(pyridin-4-yl)-4,5-dihydro-[1,2,4]triazolo[4,3-f]pteridine (Example 55),
(R)-4-ethyl-5-isopropyl-1-methyl-7-(pyridin-4-yl)-4,5-dihydro-[1,2,4]triazolo[4,3-f]pteridine (Example 56),
(R)-7-(pyridin-4-yl)-11,12,13,13a-tetrahydro-10H-pyrido[2,1-h][1,2,4]triazolo[4,3-f]pteridine (Example 59),
(R)-3-methyl-7-(pyridin-4-yl)-11,12,13,13a-tetrahydro-10H-pyrido[2,1-h][1,2,4]triazolo[4,3-f]pteridine (Example 60),
(R)-4-ethyl-5-isopropyl-7-(1H-pyrazol-4-yl)-4,5-dihydro-[1,2,4]triazolo[4,3-f]pteridine (Example 69),
(R)-4-ethyl-5-isopropyl-1-methyl-7-(1H-pyrazol-4-yl)-4,5-dihydro-[1,2,4]triazolo[4,3-f]pteridine (Example 70),
(R)-5-cyclopentyl-4-ethyl-7-(1H-pyrrolo[2,3-b]pyridin-5-yl)-4,5-dihydro-[1,2,4]triazolo[4,3-f]pteridine (Example 71),
(R)-5-cyclopentyl-4-ethyl-1-methyl-7-(1H-pyrrolo[2,3-b]pyridin-5-yl)-4,5-dihydro-[1,2,4]triazolo[4,3-f]pteridine (Example 72),
(R)-7-(pyridin-4-yl)-10,11,12,12a-tetrahydropyrrolo[2,1-h][1,2,4]triazolo[4,3-f]pteridine (Example 75),
(R)-3-methyl-7-(pyridin-4-yl)-10,11,12,12a-tetrahydropyrrolo[2,1-h][1,2,4]triazolo[4,3-f]pteridine (Example 76),
(R)-N-(3-(5-cyclopentyl-4-ethyl-4,5-dihydro-[1,2,4]triazolo[4,3-f]pteridin-7-yl)phenyl)methanesulfonamide (Example 195),
(R)-N-(3-(5-cyclopentyl-4-ethyl-1-methyl-4,5-dihydro-[1,2,4]triazolo[4,3-f]pteridin-7-yl)phenyl)methanesulfonamide (Example 196),
(R)-3-(5-cyclopentyl-4-ethyl-4,5-dihydro-[1,2,4]triazolo[4,3-f]pteridin-7-yl)-N,N-dimethylbenzamide (Example 197),
(R)-3-(5-cyclopentyl-4-ethyl-1-methyl-4,5-dihydro-[1,2,4]triazolo[4,3-f]pteridin-7-yl)-N,N-dimethylbenzamide (Example 198),
(R)-5-cyclopentyl-4-ethyl-7-(4-(methylsulfonyl)phenyl)-4,5-dihydro-[1,2,4]triazolo[4,3-f]pteridine (Example 199),
(R)-5-cyclopentyl-4-ethyl-1-methyl-7-(4-(methylsulfonyl)phenyl)-4,5-dihydro-[1,2,4]triazolo[4,3-f]pteridine (Example 200),
(R)-3-(5-cyclopentyl-4-ethyl-4,5-dihydro-[1,2,4]triazolo[4,3-f]pteridin-7-yl)benzamide (Example 201),
(R)-3-(5-cyclopentyl-4-ethyl-1-methyl-4,5-dihydro-[1,2,4]triazolo[4,3-f]pteridin-7-yl)benzamide (Example 202),
(R)-7-(biphenyl-2-yl)-5-cyclopentyl-4-ethyl-4,5-dihydro-[1,2,4]triazolo[4,3-f]pteridine (Example 203),
(R)-7-(biphenyl-2-yl)-5-cyclopentyl-4-ethyl-1-methyl-4,5-dihydro-[1,2,4]triazolo[4,3-f]pteridine (Example 204),
(R)-5-cyclopentyl-4-ethyl-7-(3-(methylsulfonyl)phenyl)-4,5-dihydro-[1,2,4]triazolo[4,3-f]pteridine (Example 205),
(R)-5-cyclopentyl-4-ethyl-1-methyl-7-(3-(methylsulfonyl)phenyl)-4,5-dihydro-[1,2,4]triazolo[4,3-f]pteridine (Example 206),
(R)-7-(3-(benzyloxy)phenyl)-5-cyclopentyl-4-ethyl-4,5-dihydro-[1,2,4]triazolo[4,3-f]pteridine (Example 207),
(R)-7-(3-(benzyloxy)phenyl)-5-cyclopentyl-4-ethyl-1-methyl-4,5-dihydro-[1,2,4]triazolo[4,3-f]pteridine (Example 208),
(R)-5-cyclopentyl-4-ethyl-7-(3-phenylpyridin-4-yl)-4,5-dihydro-[1,2,4]triazolo[4,3-f]pteridine (Example 209),
(R)-5-cyclopentyl-4-ethyl-1-methyl-7-(3-phenylpyridin-4-yl)-4,5-dihydro-[1,2,4]triazolo[4,3-f]pteridine (Example 210),
(R)-5-cyclobutyl-4-ethyl-7-(3-phenylpyridin-4-yl)-4,5-dihydro-[1,2,4]triazolo[4,3-f]pteridine (Example 211),
(R)-5-cyclobutyl-4-ethyl-1-methyl-7-(3-phenylpyridin-4-yl)-4,5-dihydro-[1,2,4]triazolo[4,3-f]pteridine (Example 212),
(R)-5-cyclobutyl-4-ethyl-7-(2-(trifluoromethyl)phenyl)-4,5-dihydro-[1,2,4]triazolo[4,3-f]pteridine (Example 213),
(R)-5-cyclobutyl-4-ethyl-1-methyl-7-(2-(trifluoromethyl)phenyl)-4,5-dihydro-[1,2,4]triazolo[4,3-f]pteridine (Example 214),
(R)-5-cyclobutyl-4-ethyl-7-(pyridin-4-yl)-4,5-dihydro-[1,2,4]triazolo[4,3-f]pteridine (Example 215),
(R)-5-cyclobutyl-4-ethyl-1-methyl-7-(pyridin-4-yl)-4,5-dihydro-[1,2,4]triazolo[4,3-f]pteridine (Example 216),
(R)-4-ethyl-5-isopropyl-7-(3-phenylpyridin-4-yl)-4,5-dihydro-[1,2,4]triazolo[4,3-f]pteridine (Example 217),
(R)-4-ethyl-5-isopropyl-1-methyl-7-(3-phenylpyridin-4-yl)-4,5-dihydro-[1,2,4]triazolo[4,3-f]pteridine (Example 218),
(R)-5-cyclopropyl-4-ethyl-7-(3-phenylpyridin-4-yl)-4,5-dihydro-[1,2,4]triazolo[4,3-f]pteridine (Example 219),
(R)-5-cyclopropyl-4-ethyl-1-methyl-7-(3-phenylpyridin-4-yl)-4,5-dihydro-[1,2,4]triazolo[4,3-f]pteridine (Example 220),
(R)-4-perdeuteroethyl-5-perdeuteroisopropyl-7-(3-phenylpyridin-4-yl)-4,5-dihydro-[1,2,4]triazolo[4,3-f]pteridine (Example 221),
(R)-4-perdeuteroethyl-5-perdeuteroisopropyl-1-methyl-7-(3-phenylpyridin-4-yl)-4,5-dihydro-[1,2,4]triazolo[4,3-f]pteridine (Example 222),
(R)-2-(4-(4-ethyl-5-(tetrahydro-2H-pyran-4-yl)-4,5-dihydro-[1,2,4]triazolo[4,3-f]pteridin-7-yl)-1H-pyrazol-5-yl)thiazole (Example 223),
(R)-2-(4-(4-ethyl-1-methyl-5-(tetrahydro-2H-pyran-4-yl)-4,5-dihydro-[1,2,4]triazolo[4,3-f]pteridin-7-yl)-1H-pyrazol-5-yl)thiazole (Example 224),
(R)-2-(4-(5-(3,3-difluorocyclobutyl)-4-ethyl-4,5-dihydro-[1,2,4]triazolo[4,3-f]pteridin-7-yl)-1H-pyrazol-5-yl)thiazole (Example 225),
(R)-2-(4-(5-(3,3-difluorocyclobutyl)-4-ethyl-1-methyl-4,5-dihydro-[1,2,4]triazolo[4,3-f]pteridin-7-yl)-1H-pyrazol-5-yl)thiazole (Example 226),
2-(4-((4R)-S-(1-cyclopropylethyl)-4-ethyl-4,5-dihydro-[1,2,4]triazolo[4,3-f]pteridin-7-yl)-1H-pyrazol-5-yl)thiazole (Example 227),
2-(4-((4R)-S-(1-cyclopropylethyl)-4-ethyl-1-methyl-4,5-dihydro-[1,2,4]triazolo[4,3-f]pteridin-7-yl)-1H-pyrazol-5-yl)thiazole (Example 228),
(R)-2-(4-(4-ethyl-5-(1H-pyrazol-4-yl)-4,5-dihydro-[1,2,4]triazolo[4,3-f]pteridin-7-yl)-1H-pyrazol-5-yl)thiazole, (S)-2-(4-(4-ethyl-5-(1H-pyrazol-4-yl)-4,5-dihydro-[1,2,4]triazolo[4,3-f]pteridin-7-yl)-1H-pyrazol-5-yl)thiazole (Example 229),
(R)-2-(4-(4-ethyl-1-methyl-5-(1H-pyrazol-4-yl)-4,5-dihydro-[1,2,4]triazolo[4,3-f]pteridin-7-yl)-1H-pyrazol-5-yl)thiazole, (S)-2-(4-(4-ethyl-1-methyl-5-(1H-pyrazol-4-yl)-4,5-dihydro-[1,2,4]triazolo[4,3-f]pteridin-7-yl)-1H-pyrazol-5-yl)thiazole (Example 230),
2-(4-((4R)-4-ethyl-5-(tetrahydrofuran-3-yl)-4,5-dihydro-[1,2,4]triazolo[4,3-f]pteridin-7-yl)-1H-pyrazol-5-yl)thiazole (Example 231),
2-(4-((4R)-4-ethyl-1-methyl-5-(tetrahydrofuran-3-yl)-4,5-dihydro-[1,2,4]triazolo[4,3-f]pteridin-7-yl)-1H-pyrazol-5-yl)thiazole (Example 232), 2-(4-(4-ethyl-5-(4-fluorophenyl)-4,5-dihydro-[1,2,4]triazolo[4,3-f]pteridin-7-yl)-1H-pyrazol-5-yl)thiazole (Example 233),
2-(4-(4-ethyl-5-(4-fluorophenyl)-1-methyl-4,5-dihydro-[1,2,4]triazolo[4,3-f]pteridin-7-yl)-1H-pyrazol-5-yl)thiazole (Example 234),
3-(4-ethyl-7-(5-(thiazol-2-yl)-1H-pyrazol-4-yl)-[1,2,4]triazolo[4,3-f]pteridin-5(4H)-yl)benzonitrile (Example 235),
3-(4-ethyl-1-methyl-7-(5-(thiazol-2-yl)-1H-pyrazol-4-yl)-[1,2,4]triazolo[4,3-f]pteridin-5(4H)-yl)benzonitrile (Example 236),
(R)-2-(4-(5-(4-chlorophenyl)-4-ethyl-4,5-dihydro-[1,2,4]triazolo[4,3-f]pteridin-7-yl)-1H-pyrazol-5-yl)thiazole and (S)-2-(4-(5-(4-chlorophenyl)-4-ethyl-4,5-dihydro-[1,2,4]triazolo[4,3-f]pteridin-7-yl)-1H-pyrazol-5-yl)thiazole (Example 237),
(R)-2-(4-(5-(4-chlorophenyl)-4-ethyl-1-methyl-4,5-dihydro-[1,2,4]triazolo[4,3-f]pteridin-7-yl)-1H-pyrazol-5-yl)thiazole and (S)-2-(4-(5-(4-chlorophenyl)-4-ethyl-1-methyl-4,5-dihydro-[1,2,4]triazolo[4,3-f]pteridin-7-yl)-1H-pyrazol-5-yl)thiazole (Example 238),
(R)-2-(4-(5-(3,4-difluorophenyl)-4-ethyl-4,5-dihydro-[1,2,4]triazolo[4,3-f]pteridin-7-yl)-1H-pyrazol-5-yl)thiazole and (S)-2-(4-(5-(3,4-difluorophenyl)-4-ethyl-4,5-dihydro-[1,2,4]triazolo[4,3-f]pteridin-7-yl)-1H-pyrazol-5-yl)thiazole (Example 239),
(R)-2-(4-(5-(3,4-difluorophenyl)-4-ethyl-1-methyl-4,5-dihydro-[1,2,4]triazolo[4,3-f]pteridin-7-yl)-1H-pyrazol-5-yl)thiazole and (S)-2-(4-(5-(3,4-difluorophenyl)-4-ethyl-1-methyl-4,5-dihydro-[1,2,4]triazolo[4,3-f]pteridin-7-yl)-1H-pyrazol-5-yl)thiazole (Example 240),
(R)-13a-ethyl-7-(3-phenylpyridin-4-yl)-10,11,13,13a-tetrahydro-[1,4]oxazino[3,4-h][1,2,4]triazolo[4,3-f]pteridine and (S)-13a-ethyl-3-7-(3-phenylpyridin-4-yl)-10,11,13,13a-tetrahydro-[1,4]oxazino[3,4-h][1,2,4]triazolo[4,3-f]pteridine (Example 241),
(R)-13a-ethyl-3-methyl-7-(3-phenylpyridin-4-yl)-10,11,13,13a-tetrahydro-[1,4]oxazino[3,4-h][1,2,4]triazolo[4,3-f]pteridine and (S)-13a-ethyl-3-methyl-7-(3-phenylpyridin-4-yl)-10,11,13,13a-tetrahydro-[1,4]oxazino[3,4-h][1,2,4]triazolo[4,3-f]pteridine (Example 242),
4-ethyl-5-(1-methyl-1H-pyrazol-4-yl)-7-(5-(pyridin-2-yl)-1H-pyrazol-4-yl)-4,5-dihydro-[1,2,4]triazolo[4,3-f]pteridine (Example 697), 4-ethyl-1-methyl-5-(1-methyl-1H-pyrazol-4-yl)-7-(5-(pyridin-2-yl)-1H-pyrazol-4-yl)-4,5-dihydro-[1,2,4]triazolo[4,3-f]pteridine (Example 698),
2-(4-(5-(1-(cyclopropylmethyl)-1H-pyrazol-4-yl)-4-ethyl-4,5-dihydro-[1,2,4]triazolo[4,3-f]pteridin-7-yl)-1H-pyrazol-5-yl)thiazole (Example 699), 2-(4-(5-(1-(cyclopropylmethyl)-1H-pyrazol-4-yl)-4-ethyl-1-methyl-4,5-dihydro-[1,2,4]triazolo[4,3-f]pteridin-7-yl)-1H-pyrazol-5-yl)thiazole (Example 700),
(R)-13a-ethyl-7-(2-phenylpyridin-3-yl)-10,11,13,13a-tetrahydro-[1,4]oxazino[3,4-h][1,2,4]triazolo[4,3-f]pteridine, (S)-13a-ethyl-7-(2-phenylpyridin-3-yl)-10,11,13,13a-tetrahydro-[1,4]oxazino[3,4-h][1,2,4]triazolo[4,3-f]pteridine (Example 703),
(R)-13a-ethyl-3-methyl-7-(2-phenylpyridin-3-yl)-10,11,13,13a-tetrahydro-[1,4]oxazino[3,4-h][1,2,4]triazolo[4,3-f]pteridine, (S)-13a-ethyl-3-methyl-7-(2-phenylpyridin-3-yl)-10,11,13,13a-tetrahydro-[1,4]oxazino[3,4-h][1,2,4]triazolo[4,3-f]pteridine (Example 704),
(R)-2-(4-(4-ethyl-5-(1-methyl-1H-pyrazol-4-yl)-4,5-dihydro-[1,2,4]triazolo[4,3-f]pteridin-7-yl)-1-methyl-1H-pyrazol-3-yl)thiazole (Example 705),
(R)-2-(4-(4-ethyl-1-methyl-5-(1-methyl-1H-pyrazol-4-yl)-4,5-dihydro-[1,2,4]triazolo[4,3-f]pteridin-7-yl)-1-methyl-1H-pyrazol-3-yl)thiazole (Example 706),
(R)-7-(3-(2,4-difluorophenyl)-1H-pyrazol-4-yl)-4-ethyl-5-(1-methyl-1H-pyrazol-4-yl)-4,5-dihydro-[1,2,4]triazolo[4,3-f]pteridine (Example 707),
(R)-7-(3-(2,4-difluorophenyl)-1H-pyrazol-4-yl)-4-ethyl-1-methyl-5-(1-methyl-1H-pyrazol-4-yl)-4,5-dihydro-[1,2,4]triazolo[4,3-f]pteridine (Example 708),
7-(3-(2,4-difluorophenyl)-1H-pyrazol-4-yl)-4-ethyl-5-(1-methyl-1H-pyrazol-3-yl)-4,5-dihydro-[1,2,4]triazolo[4,3-f]pteridine (Example 709),
7-(3-(2,4-difluorophenyl)-1H-pyrazol-4-yl)-4-ethyl-1-methyl-5-(1-methyl-1H-pyrazol-3-yl)-4,5-dihydro-[1,2,4]triazolo[4,3-f]pteridine (Example 710),
(R)-2-(4-(4-ethyl-5-(1-methyl-1H-pyrazol-3-yl)-4,5-dihydro-[1,2,4]triazolo[4,3-f]pteridin-7-yl)-1-methyl-1H-pyrazol-3-yl)thiazole (Example 711),
(R)-2-(4-(4-ethyl-1-methyl-5-(1-methyl-1H-pyrazol-3-yl)-4,5-dihydro-[1,2,4]triazolo[4,3-f]pteridin-7-yl)-1-methyl-1H-pyrazol-3-yl)thiazole (Example 712),
(R)-2-(4-(4-ethyl-5-(1-methyl-1H-pyrazol-3-yl)-4,5-dihydro-[1,2,4]triazolo[4,3-f]pteridin-7-yl)-1-methyl-1H-pyrazol-5-yl)thiazole (Example 713),
(R)-2-(4-(4-ethyl-1-methyl-5-(1-methyl-1H-pyrazol-3-yl)-4,5-dihydro-[1,2,4]triazolo[4,3-f]pteridin-7-yl)-1-methyl-1H-pyrazol-5-yl)thiazole (Example 714),
2-(4-(4-ethyl-5-(1-methyl-1H-pyrazol-4-yl)-4,5-dihydro-[1,2,4]triazolo[4,3-f]pteridin-7-yl)-1H-pyrazol-5-yl)thiazole (Example 715),
2-(4-(4-ethyl-1-methyl-5-(1-methyl-1H-pyrazol-4-yl)-4,5-dihydro-[1,2,4]triazolo[4,3-f]pteridin-7-yl)-1H-pyrazol-5-yl)thiazole (Example 716),
(R)-4-ethyl-5-(1-methyl-1H-pyrazol-3-yl)-7-(3-phenylpyridin-4-yl)-4,5-dihydro-[1,2,4]triazolo[4,3-f]pteridine (Example 717),
(R)-4-ethyl-1-methyl-5-(1-methyl-1H-pyrazol-3-yl)-7-(3-phenylpyridin-4-yl)-4,5-dihydro-[1,2,4]triazolo[4,3-f]pteridine (Example 718),
(R)-4-ethyl-5-(1-methyl-1-pyrazol-3-yl)-7-(2-phenylpyridin-3-yl)-4,5-dihydro-[1,2,4]triazolo[4,3-f]pteridine (Example 719),
(R)-4-ethyl-1-methyl-5-(1-methyl-1H-pyrazol-3-yl)-7-(2-phenylpyridin-3-yl)-4,5-dihydro-[1,2,4]triazolo[4,3-f]pteridine (Example 720),
(R)-4-ethyl-5-(1-methyl-1-pyrazol-4-yl)-7-(2-phenylpyridin-3-yl)-4,5-dihydro-[1,2,4]triazolo[4,3-f]pteridine (Example 721),
(R)-4-ethyl-1-methyl-5-(1-methyl-1H-pyrazol-4-yl)-7-(2-phenylpyridin-3-yl)-4,5-dihydro-[1,2,4]triazolo[4,3-f]pteridine (Example 722),
(R)-4-ethyl-5-(1-methyl-1H-pyrazol-4-yl)-7-(3-phenylpyridin-4-yl)-4,5-dihydro-[1,2,4]triazolo[4,3-f]pteridine (Example 723),
(R)-4-ethyl-1-methyl-5-(1-methyl-1H-pyrazol-4-yl)-7-(3-phenylpyridin-4-yl)-4,5-dihydro-[1,2,4]triazolo[4,3-f]pteridine (Example 724),
4-(4-ethyl-7-(5-(thiazol-2-yl)-1H-pyrazol-4-yl)-[1,2,4]triazolo[4,3-f]pteridin-5(4H)-yl)benzonitrile (Example 725),
4-(4-ethyl-1-methyl-7-(5-(thiazol-2-yl)-1H-pyrazol-4-yl)-[1,2,4]triazolo[4,3-f]pteridin-5(4H)-yl)benzonitrile (Example 726),
4-(4-ethyl-7-(5-(thiazol-2-yl)-1H-pyrazol-4-yl)-[1,2,4]triazolo[4,3-f]pteridin-5(4H)-yl)benzamide (Example 727),
4-(4-ethyl-1-methyl-7-(5-(thiazol-2-yl)-1H-pyrazol-4-yl)-[1,2,4]triazolo[4,3-f]pteridin-5(4H)-yl)benzamide (Example 728),
(R)-2-(4-(4-ethyl-5-(tetrahydrofuran-3-yl)-4,5-dihydro-[1,2,4]triazolo[4,3-f]pteridin-7-yl)-1-methyl-1H-pyrazol-3-yl)thiazole (Example 729),
(R)-2-(4-(4-ethyl-1-methyl-5-(tetrahydrofuran-3-yl)-4,5-dihydro-[1,2,4]triazolo[4,3-f]pteridin-7-yl)-1-methyl-1H-pyrazol-3-yl)thiazole (Example 730),
(R)-2-(4-(4-ethyl-5-(tetrahydrofuran-3-yl)-4,5-dihydro-[1,2,4]triazolo[4,3-f]pteridin-7-yl)-1-methyl-1H-pyrazol-5-yl)thiazole (Example 731), and
(R)-2-(4-(4-ethyl-1-methyl-5-(tetrahydrofuran-3-yl)-4,5-dihydro-[1,2,4]triazolo[4,3-f]pteridin-7-yl)-1-methyl-1H-pyrazol-5-yl)thiazole (Example 732).
   For Examples 229, 230, 697, 698, 699, 700, 707, 708, 715, 716, 725, and 726, the SEM nitrogen protecting group is removed similarly to the method of Example 655. The following table provides the example number (column 1), Intermediate (column 2), and boronic acid (column 3), to give the compound shown in column 4.

| Ex. No. | Int. | Boronic acid | Compound structure |
|---|---|---|---|
| 22 | F | 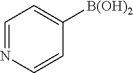 | 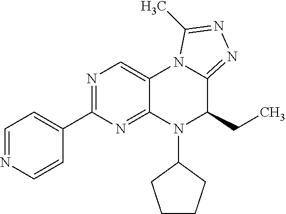 |
| 23 | E | 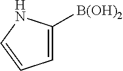 | 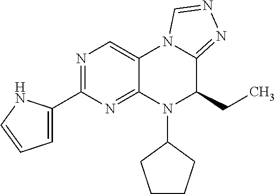 |
| 24 | F | | 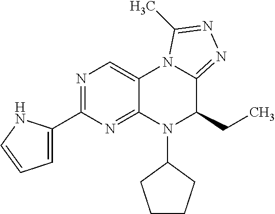 |
| 25 | E | 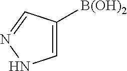 | 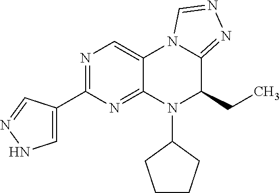 |
| 26 | F | | 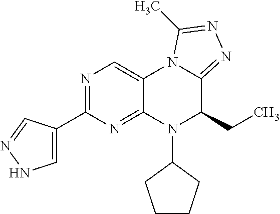 |
| 27 | E | 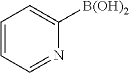 | 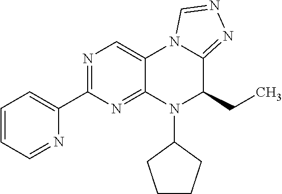 |
| 28 | F | | 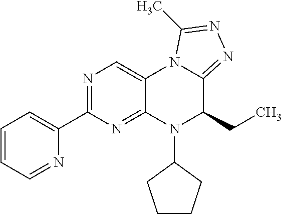 |
| 29 | E | 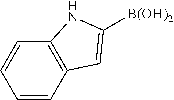 | 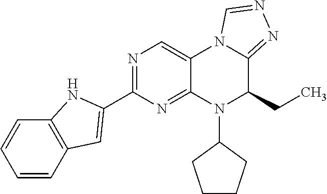 |
| 30 | F | | 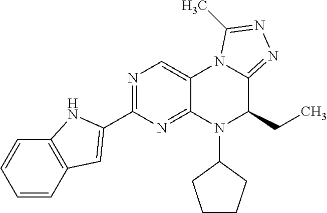 |
| 31 | E | 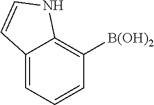 | 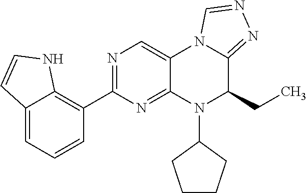 |
| 32 | F | | 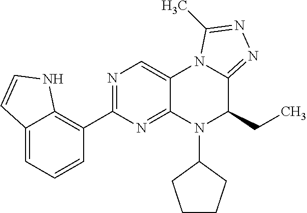 |
| 33 | E | 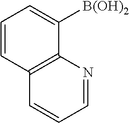 | 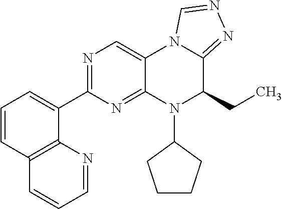 |
| 34 | F | | 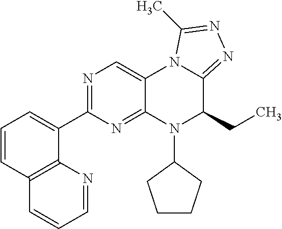 |
| 35 | E | 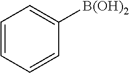 | 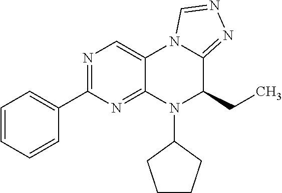 |
| 36 | F | | 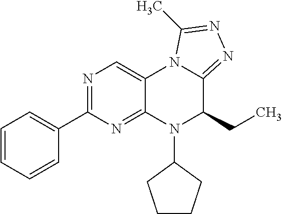 |
| 39 | E | 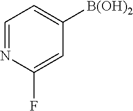 | 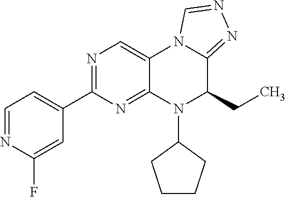 |
| 40 | F | | 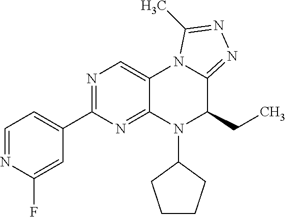 |
| 55 | G | 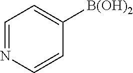 | 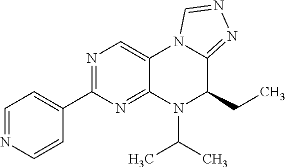 |
| 56 | H | | 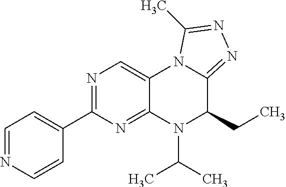 |
| 59 | I | 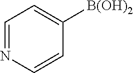 | 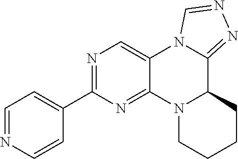 |
| 60 | J | | 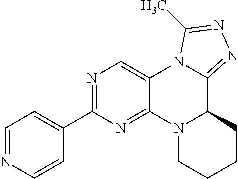 |
| 69 | G | 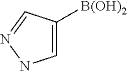 | 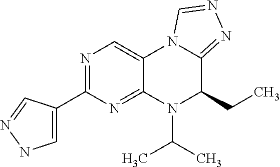 |
| 70 | H | | 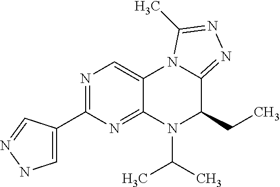 |
| 71 | E | 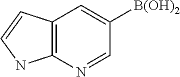 | 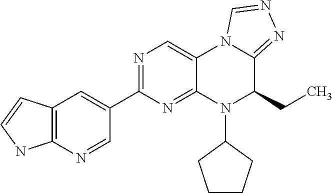 |
| 72 | F | | 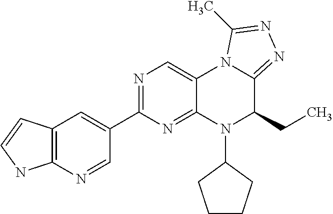 |
| 75 | K | 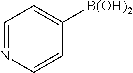 | 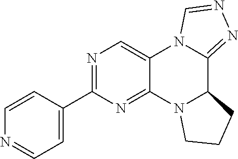 |
| 76 | L | | 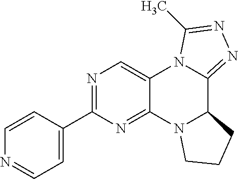 |
| 195 | E | 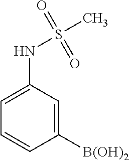 | 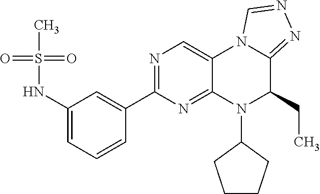 |
| 196 | F | | 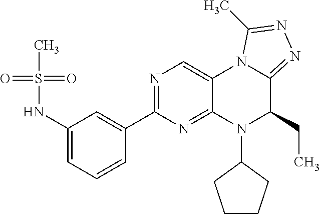 |
| 197 | E | 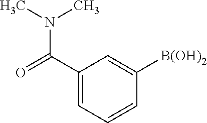 | 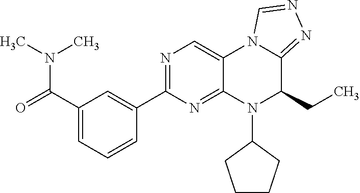 |
| 198 | F | | 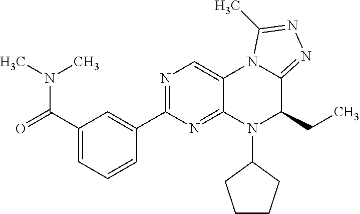 |
| 199 | E | 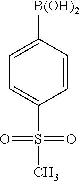 | 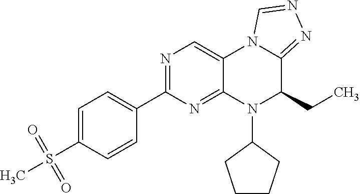 |
| 200 | F | | 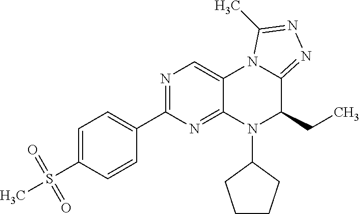 |
| 201 | E | 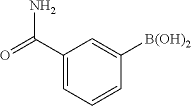 | 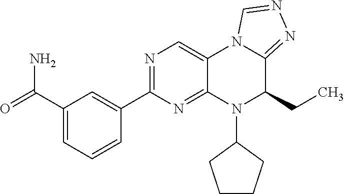 |
| 202 | F | | 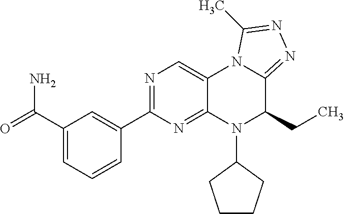 |
| 203 | E | 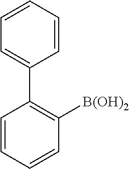 | 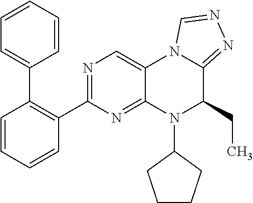 |
| 204 | F | | 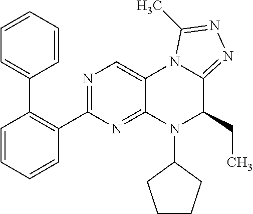 |
| 205 | E | 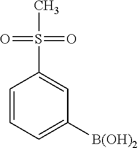 | 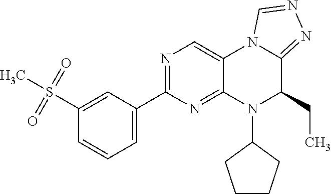 |
| 206 | F | | 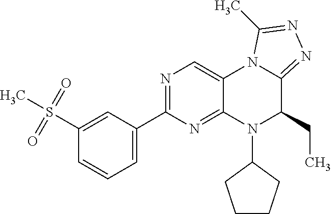 |
| 207 | E | 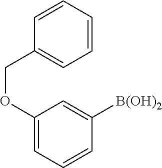 | 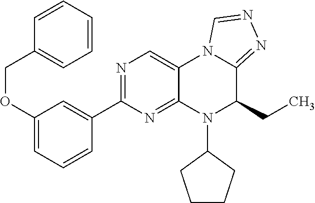 |
| 208 | F | | 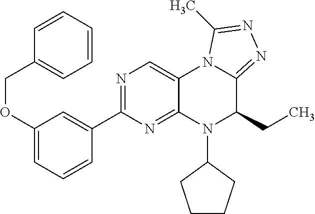 |
| 209 | E | 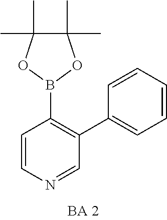 | 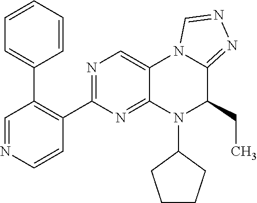 |
| 210 | F | | 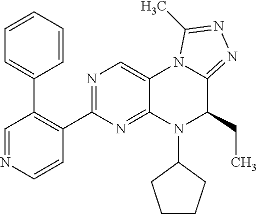 |
| 211 | C | 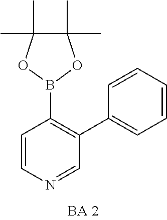 | 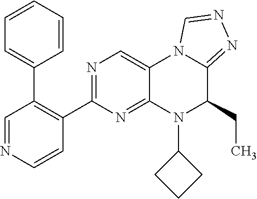 |
| 212 | D | | 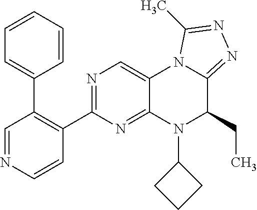 |
| 213 | C | 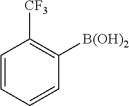 | 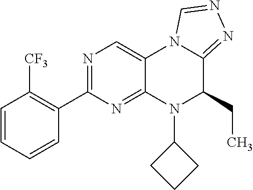 |
| 214 | D | | 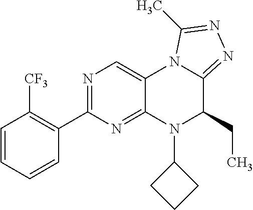 |
| 215 | C | 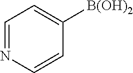 | 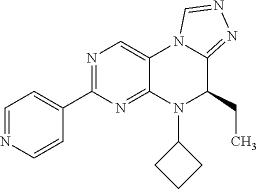 |
| 216 | D | | 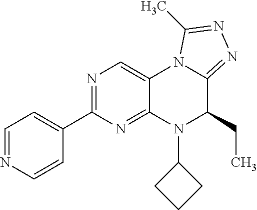 |
| 217 | G | 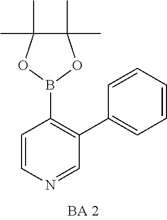 | 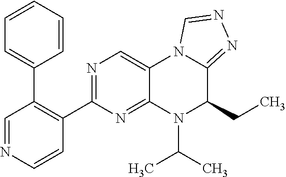 |
| 218 | H | | 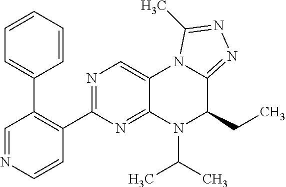 |
| 219 | O | 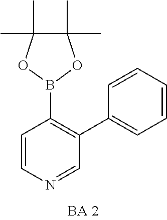 | 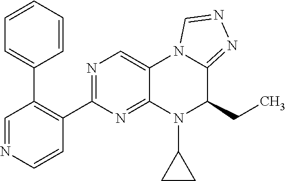 |
| 220 | O′ | | 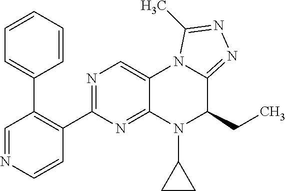 |
| 221 | Q | 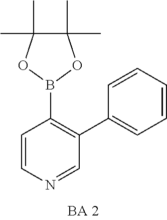 | 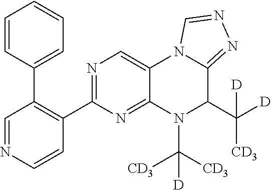 |
| 222 | Q′ | | 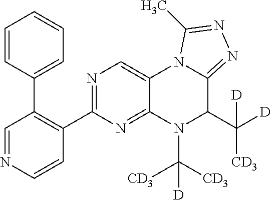 |
| 223 | M | 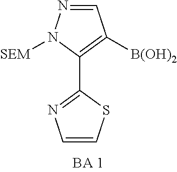 | 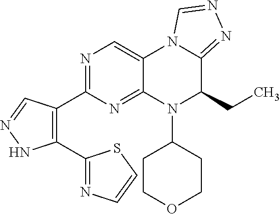 |
| 224 | M′ | | 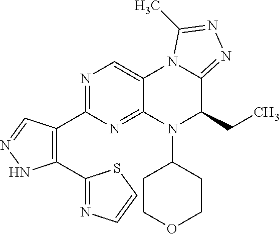 |
| 225 | V | 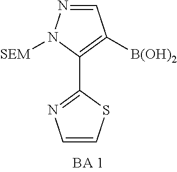 | 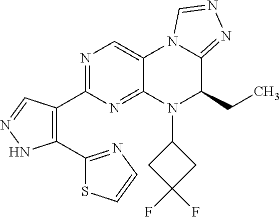 |
| 226 | V′ | | 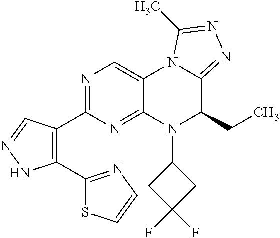 |
| 227 | SS | 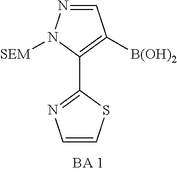 | 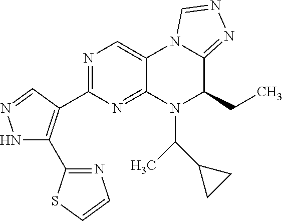 |
| 228 | SS′ | | 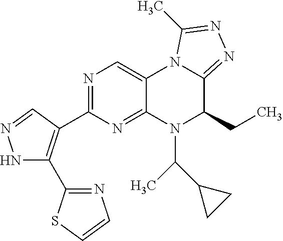 |
| 229(R) | KK | 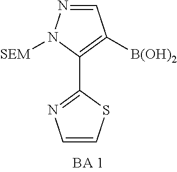 | 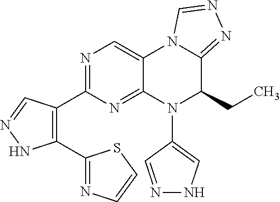 |
| 229(S) | | | 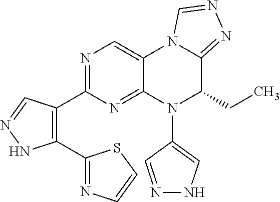 |
| 230(R) | KK′ | 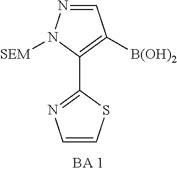 | 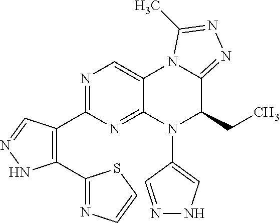 |
| 230(S) | | | 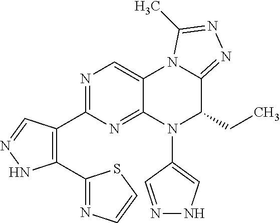 |
| 231 | N | 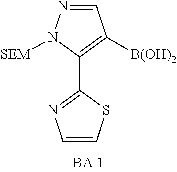 | 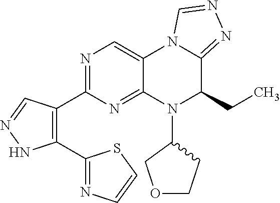 |
| 232 | N′ | | 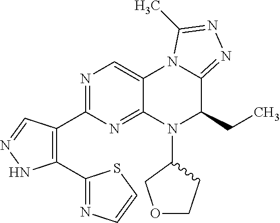 |
| 233 | EE | 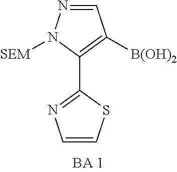 | 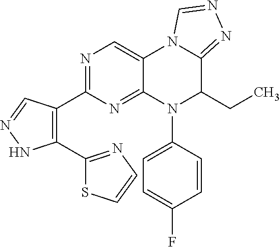 |
| 234 | EE′ | | 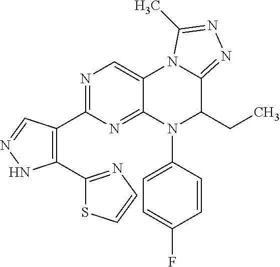 |
| 235 | OO | 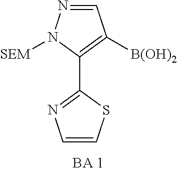 | 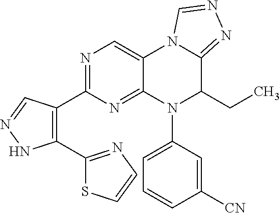 |
| 236 | OO′ | | 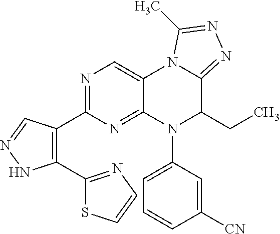 |
| 237(R) | TT | 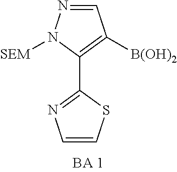 | 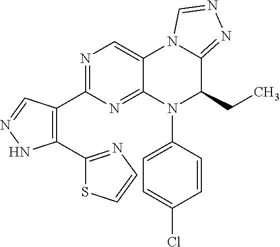 |
| 237(S) | | | 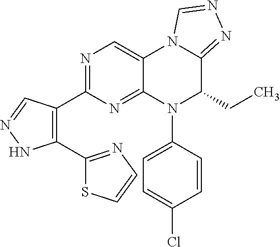 |
| 238(R) | TT′ | 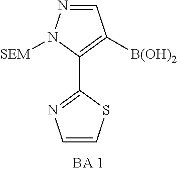 | 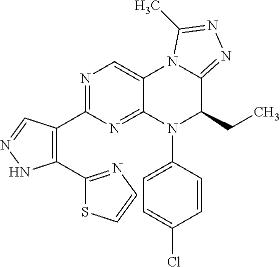 |
| 238(S) | | | 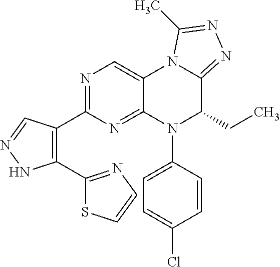 |
| 239(R) | UU | 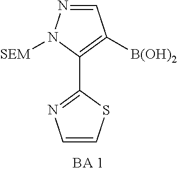 | 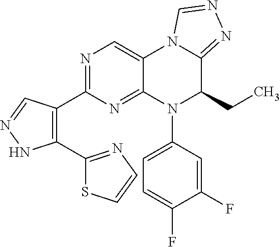 |
| 239(S) | | | 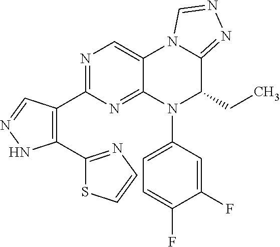 |
| 240(R) | UU′ | 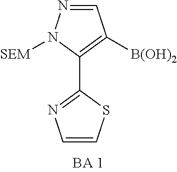 | 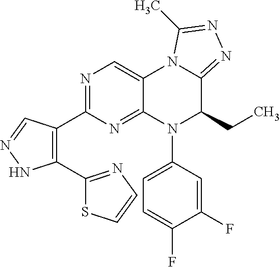 |
| 240(S) | | | 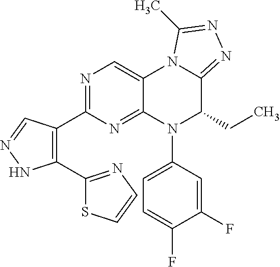 |
| 241(R) | Z | 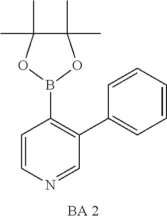 | 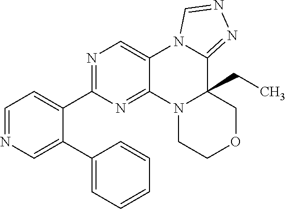 |
| 241(S) | | | 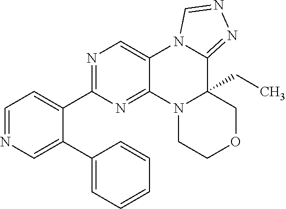 |
| 242(R) | Z′ | 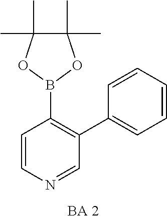 | 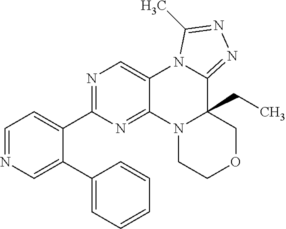 |
| 242(S) | | | 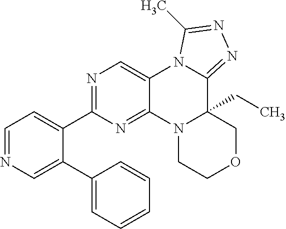 |
| 697 | KK-3 | 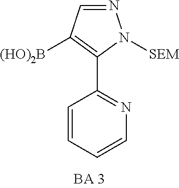 | 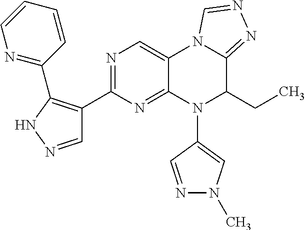 |
| 698 | KK′-3 | | 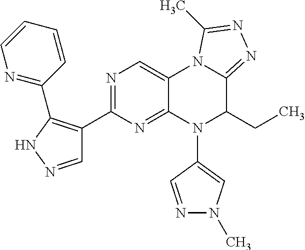 |
| 699 | KK-4 | 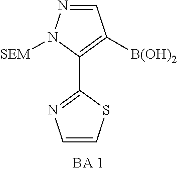 | 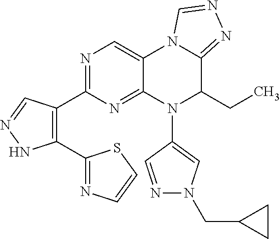 |
| 700 | KK′-4 | | 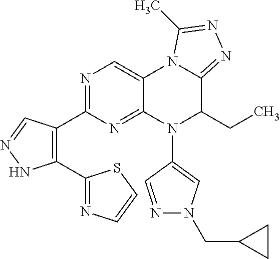 |
| 703(R) | Z | 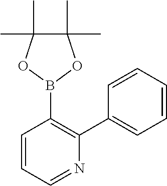 | 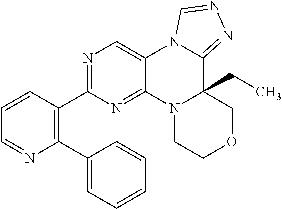 |
| 703(S) | | | 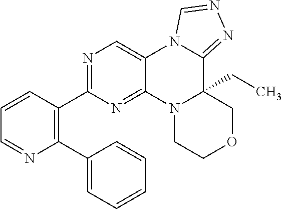 |
| 704(R) | Z′ | 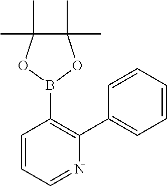 | 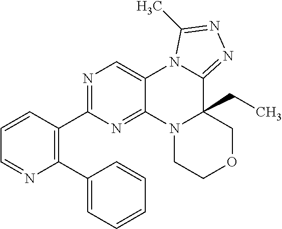 |
| 704(S) | | | 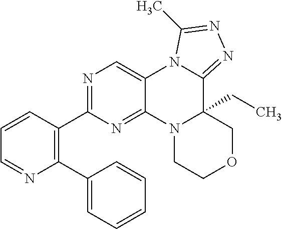 |
| 705 | KK-2 | 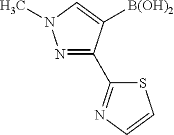 | 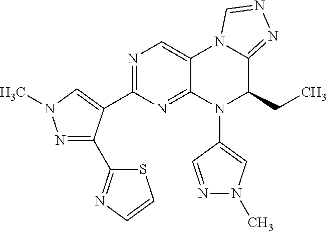 |
| 706 | KK′-2 | | 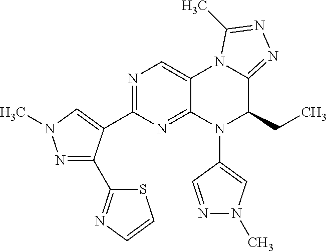 |
| 707 | KK-2 | 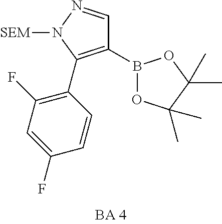 | 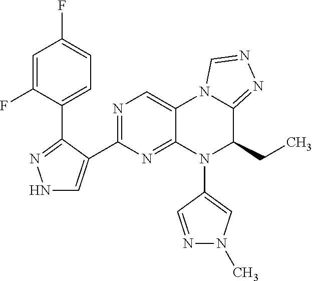 |
| 708 | KK′-2 | | 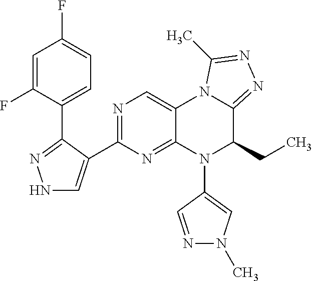 |
| 709 | QQ-2 | 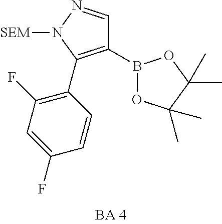 | 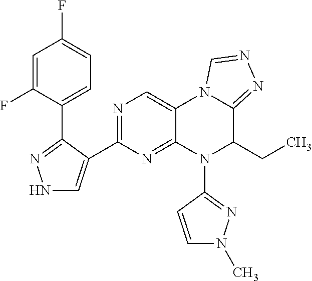 |
| 710 | QQ′-2 | | 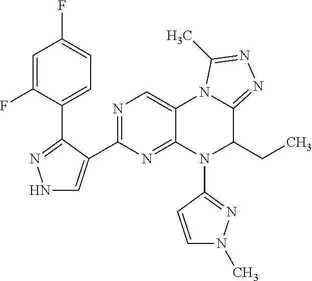 |
| 711 | QQ-2 | 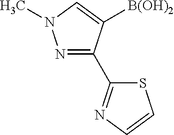 | 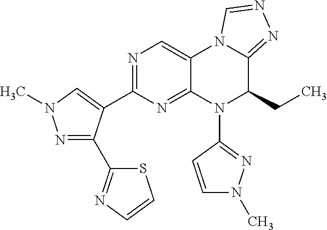 |
| 712 | QQ′-2 | | 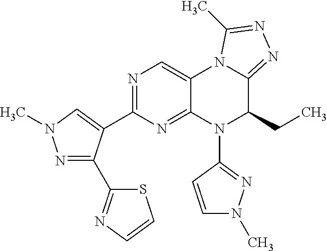 |
| 713 | QQ-2 | 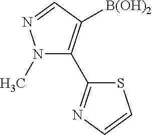 | 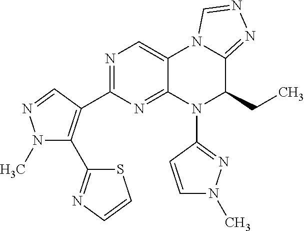 |
| 714 | QQ′-2 | | 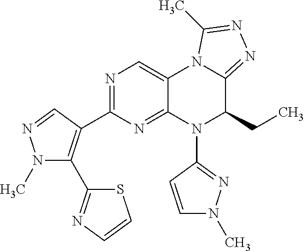 |
| 715 | KK-3 | 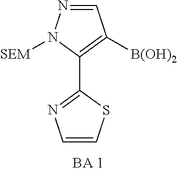 | 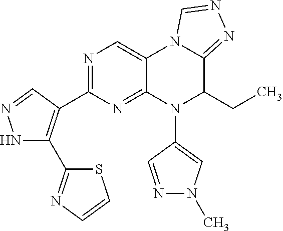 |
| 716 | KK′-3 | | 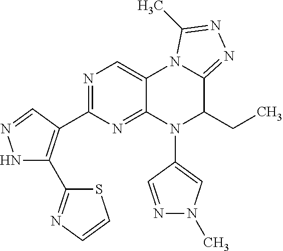 |
| 717 | QQ-2 | 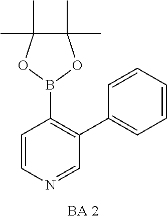 | 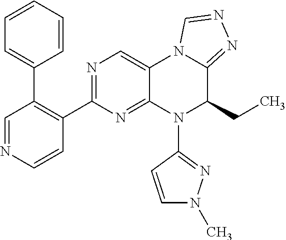 |
| 718 | QQ′-2 | | 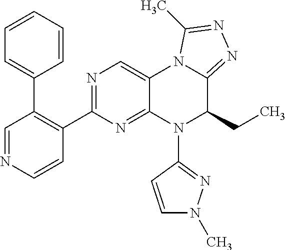 |
| 719 | QQ-2 | 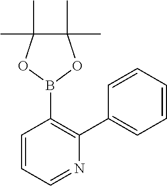 | 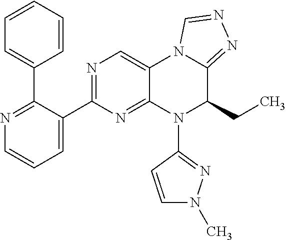 |
| 720 | QQ′-2 | | 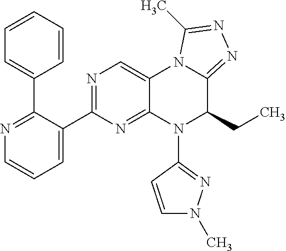 |
| 721 | KK_2 | 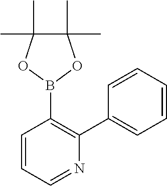 | 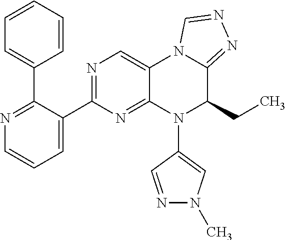 |
| 722 | KK′-2 | | 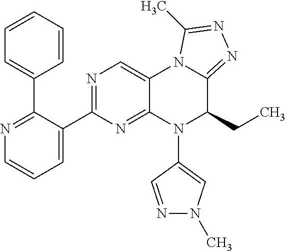 |
| 723 | KK-2 | 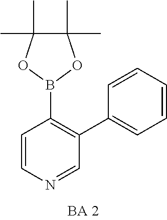 | 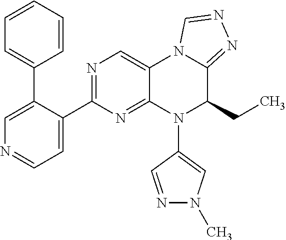 |
| 724 | KK′-2 | | 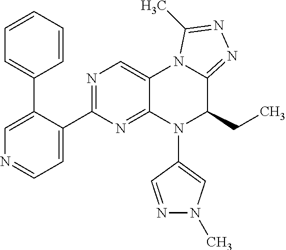 |
| 725 | PP | 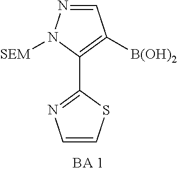 | 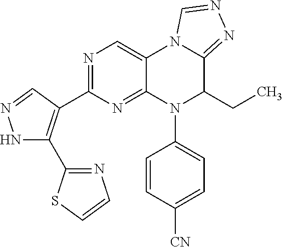 |
| 726 | PP′ | | 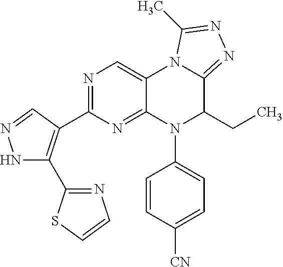 |
| 727 | Isolated side product in synthesis of Examples 725 and 726 | | 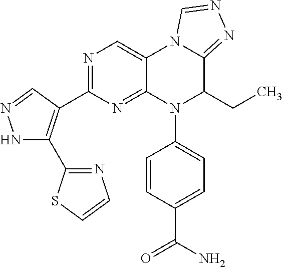 |
| 728 | | | 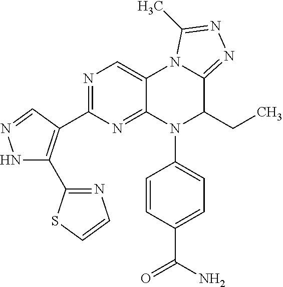 |
| 729 | N | 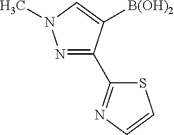 | 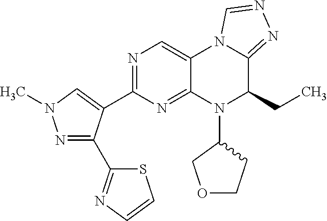 |
| 730 | N′ | | 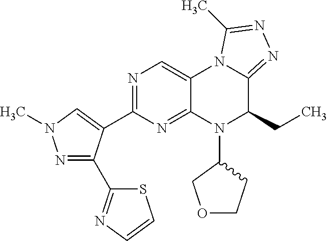 |
| 731 | N | 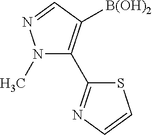 | 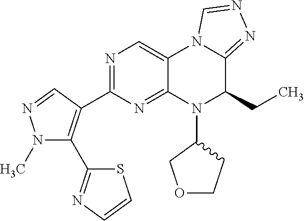 |
| 732 | N′ | | 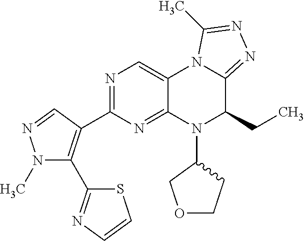 |

### Example 37

### Synthesis of (R)-4-(5-cyclopentyl-4-ethyl-4,5-dihydro-[1,2,4]triazolo[4,3-f]pteridin-7-yl)pyridine 1-oxide

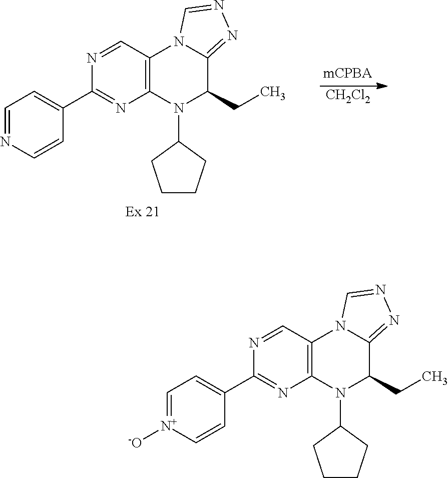

To a solution of (R)-5-cyclopentyl-4-ethyl-7-(pyridin-4-yl)-4,5-dihydro-[1,2,4]triazolo[4,3-f]pteridine (Example 21) in DCM at 0° C., mCPBA (2 eq) is added and the mixture is stirred at 0° C. for 3 hr, then at rt for another 3 hr. Saturated Na₂S₂O₄ is added and stirred at rt for 30 min. The mixture is extracted with DCM, washed with saturated NaHCO₃, concentrated and purified by prep-HPLC to give the title compound.

### Example 38

### Synthesis of (R)-4-(5-cyclopentyl-4-ethyl-1-methyl-4,5-dihydro-[1,2,4]triazolo[4,3-f]pteridin-7-yl)pyridine 1-oxide

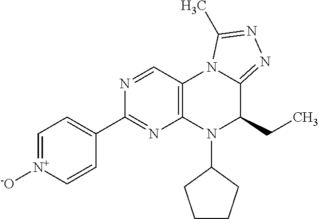

The title compound is prepared by an adaptation of the method of Example 37, with (R)-5-cyclopentyl-4-ethyl-1-methyl-7-(pyridin-4-yl)-4,5-dihydro-[1,2,4]triazolo[4,3-f]pteridine (Example 22) instead of (R)-5-cyclopentyl-4-ethyl-7-(pyridin-4-yl)-4,5-dihydro-[1,2,4]triazolo[4,3-f]pteridine.

### Example 41

### Synthesis of (R)-4-(5-cyclopentyl-4-ethyl-4,5-dihydro-[1,2,4]triazolo[4,3-f]pteridin-7-yl)pyridin-2-ol

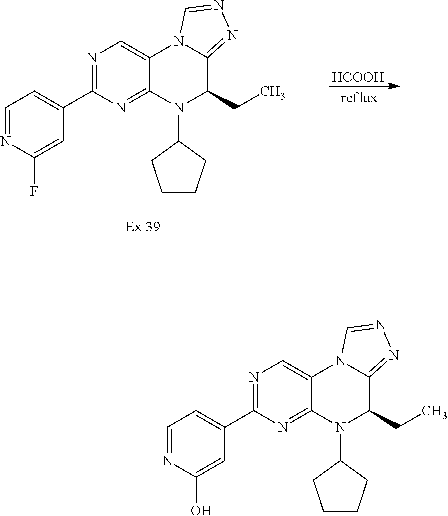

(R)-5-cyclopentyl-4-ethyl-7-(2-fluoropyridin-4-yl)-4,5-dihydro-[1,2,4]triazolo[4,3-f]pteridine (Example 39) is dissolved in HCOOH and the mixture is heated to reflux for 18 h, then aq. NaHCO₃ is added and the mixture is extracted with EtOAc. The combined organic phase is dried with Na₂SO₄, concentrated under reduced pressure and chromatographed on flash silica gel (CH₂Cl₂: CH₃OH=6:1) to give the title compound.

### Example 42

### Synthesis of (R)-4-(5-cyclopentyl-4-ethyl-1-methyl-4,5-dihydro-[1,2,4]triazolo[4,3-f]pteridin-7-yl)pyridin-2-ol

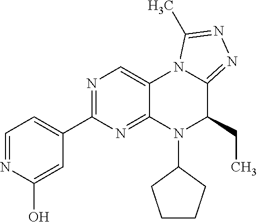

The title compound is prepared by an adaptation of the method of Example 41, with (R)-5-cyclopentyl-4-ethyl-7-(2-fluoropyridin-4-yl)-1-methyl-4,5-dihydro-[1,2,4]triazolo[4,3-f]pteridine (Example 40) instead of (R)-5-cyclopentyl-4-ethyl-7-(2-fluoropyridin-4-yl)-4,5-dihydro-[1,2,4]triazolo[4,3-f]pteridine.

### Example 43

### Synthesis of (R)-5-cyclopentyl-4-ethyl-7-(2-methoxypyridin-4-yl)-4,5-dihydro-[1,2,4]triazolo[4,3-f]pteridine

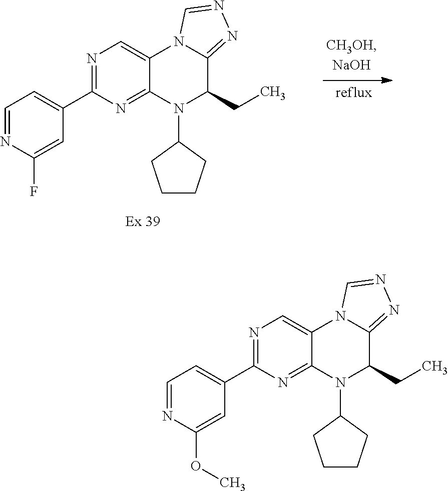

To a solution (R)-5-cyclopentyl-4-ethyl-7-(2-fluoropyridin-4-yl)-4,5-dihydro-[1,2,4]triazolo[4,3-f]pteridine (Example 39) in CH₃OH, aqueous NaOH is added and the mixture is heated to reflux overnight, concentrated under reduced pressure, and extracted with EtOAc. The combined organic phase is dried with Na₂SO₄, concentrated under reduced pressure and chromagraphed (PE: EA=1:1) to give the title compound.

### Example 44

### Synthesis of (R)-5-cyclopentyl-4-ethyl-7-(2-methoxypyridin-4-yl)-1-methyl-4,5-dihydro-[1,2,4]triazolo[4,3-f]pteridine

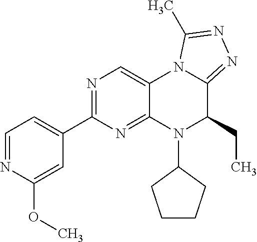

The title compound is prepared by an adaptation of the method of Example 43, with (R)-5-cyclopentyl-4-ethyl-7-(2-fluoropyridin-4-yl)-1-methyl-4,5-dihydro-[1,2,4]triazolo[4,3-f]pteridine (Example 40) instead of (R)-5-cyclopentyl-4-ethyl-7-(2-fluoropyridin-4-yl)-4,5-dihydro-[1,2,4]triazolo[4,3-f]pteridine.

### Example 47

### Synthesis of (R)-4-(5-cyclopentyl-4-ethyl-4,5-dihydro-[1,2,4]triazolo[4,3-f]pteridin-7-yl)-1-methylpyridin-2(1H)-one

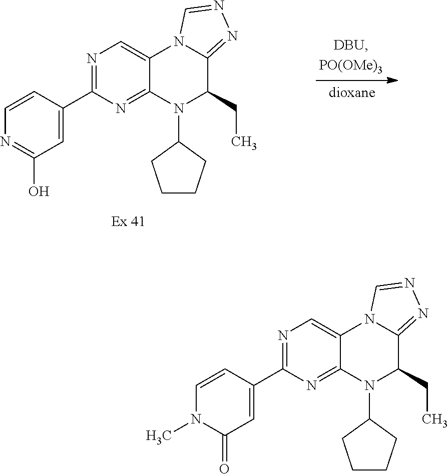

To a solution of (R)-4-(5-cyclopentyl-4-ethyl-4,5-dihydro-[1,2,4]triazolo[4,3-f]pteridin-7-yl)pyridin-2-ol (Example 41) in 1,4-dioxane, DBU (5 eq) and PO(OMe)₃ (5 eq) are added, and then the mixture is heated to reflux for 18 h, and the mixture is concentrated under reduced pressure, and extracted with EtOAc. The combined organic phase is dried with Na₂SO₄, concentrated under reduced pressure and the residue is chromatographed (PE:EA=1:1) to give the title compound.

### Example 48

### Synthesis of (R)-4-(5-cyclopentyl-4-ethyl-1-methyl-4,5-dihydro-[1,2,4]triazolo[4,3-f]pteridin-7-yl)-1-methylpyridin-2(1H)-one

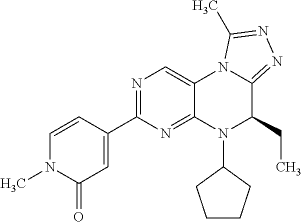

The title compound is prepared by an adaptation of the method of Example 47, with (R)-4-(5-cyclopentyl-4-ethyl-1-methyl-4,5-dihydro-[1,2,4]triazolo[4,3 4] pteridin-7-yl)pyridin-2-ol (Example 42) instead of (R)-4-(5-cyclopentyl-4-ethyl-4,5-dihydro-[1,2,4] triazolo[4,3 4] pteridin-7-yl)pyridin-2-ol.

### Example 49

### Synthesis of (R)-4-(5-cyclopentyl-4-ethyl-4,5-dihydro-[1,2,4]triazolo[4,3-f]pteridin-7-yl)-N-methylpyridin-2-amine

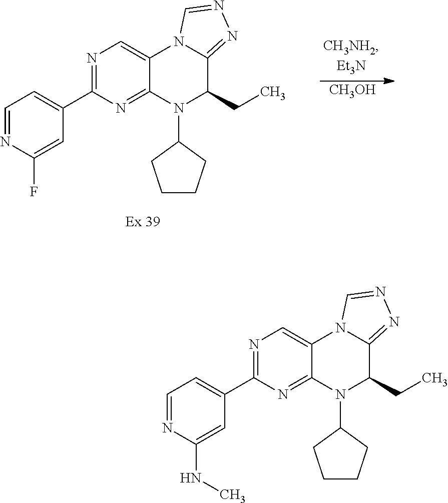

To a solution of (R)-5-cyclopentyl-4-ethyl-7-(2-fluoropyridin-4-yl)-4,5-dihydro-[1,2,4]triazolo[4,3-f]pteridine (Example 39) in methylamine (2M in CH₃OH), Et₃N is added. The mixture is stirred at 110° C. in a sealed tube for 18 h, then concentrated under reduced pressure, quenched with water and extracted with EtOAc. The combined organic phase is dried with Na₂SO₄, concentrated under reduced pressure, and chromatographed (CH₂Cl₂:CH₃OH=10:1) to give the title compound.

### Example 50

### Synthesis of (R)-4-(5-cyclopentyl-4-ethyl-1-methyl-4,5-dihydro-[1,2,4]triazolo[4,3-f]pteridin-7-yl)-N-methylpyridin-2-amine

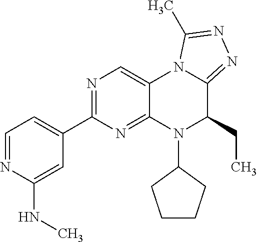

The title compound is prepared by an adaptation of the method of Example 49, with (R)-5-cyclopentyl-4-ethyl-7-(2-fluoropyridin-4-yl)-1-methyl-4,5-dihydro-[1,2,4]triazolo[4,3-f]pteridine (Example 40) instead of (R)-5-cyclopentyl-4-ethyl-7-(2-fluoropyridin-4-yl)-4,5-dihydro-[1,2,4]triazolo[4,3-f]pteridine.

### Example 51

### Synthesis of (R)-4-(5-cyclopentyl-4-ethyl-4,5-dihydro-[1,2,4]triazolo[4,3-f]pteridin-7-yl)-N,N-dimethylpyridin-2-amine

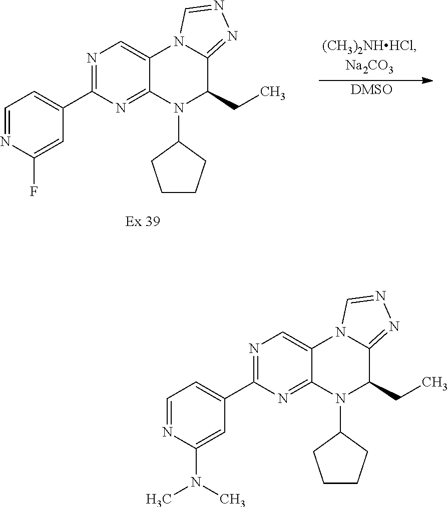

To the solution of (R)-5-cyclopentyl-4-ethyl-7-(2-fluoropyridin-4-yl)-4,5-dihydro-[1,2,4]triazolo[4,3-f]pteridine (Example 39) in DMSO, NH(Me)₂.HCl (10 eq) and Na₂CO₃ (2.1 eq) are added. The mixture is heated to 140° C. for 18 hr in a sealed tube, quenched with water and extracted with EtOAc. The combined organic phase is dried with Na₂SO₄, concentrated under reduced pressure, and chromatographed (CH₂Cl₂: CH₃OH=15:1) to give the title compound.

### Example 52

### Synthesis of (R)-4-(5-cyclopentyl-4-ethyl-1-methyl-4,5-dihydro-[1,2,4]triazolo[4,3-f]pteridin-7-yl)-N,N-dimethylpyridin-2-amine

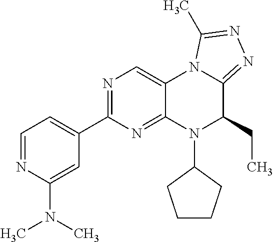

The title compound is prepared by an adaptation of the method of Example 51, with (R)-5-cyclopentyl-4-ethyl-7-(2-fluoropyridin-4-yl)-1-methyl-4,5-dihydro-[1,2,4]triazolo[4,3-f]pteridine (Example 40) instead of (R)-5-cyclopentyl-4-ethyl-7-(2-fluoropyridin-4-yl)-4,5-dihydro-[1,2,4]triazolo[4,3-f]pteridine.

### Example 61

### Synthesis of (R)-5-cyclopentyl-4-ethyl-7-(2-phenyl-1H-imidazol-1-yl)-4,5-dihydro-[1,2,4]triazolo[4,3-f]pteridine

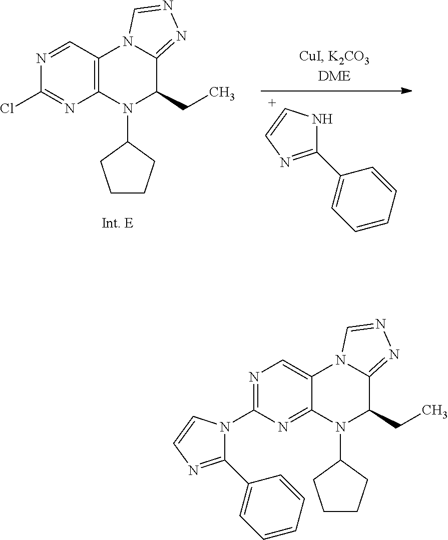

A mixture of Intermediate E, 2-phenylimidazole (20 eq), CuI (0.05 eq), 1,2-bis(methylamino) cyclohexane and K₂CO₃ in DME are heated in a microwave for 2 h at 200° C. The reaction is diluted with DME, filtered through Celite and evaporated. The residue is purified by reverse phase HPLC using a gradient of 30-50% AcCN (0.1% TFA) over 30 min with a flow rate of 20 mL/min eluting from a PCRP-5 column (2.5×30 cm).

Additional compounds are prepared similarly to this method, optionally replacing Intermediate E with a suitable intermediate, and/or replacing 2-phenyl-1H-imidazole with an appropriate ring reactant. The following compounds are prepared:
(R)-5-cyclopentyl-4-ethyl-1-methyl-7-(2-phenyl-1H-imidazol-1-yl)-4,5-dihydro-[1,2,4]triazolo[4,3-f]pteridine (Example 62),
(R)-5-cyclopentyl-4-ethyl-7-(2-methyl-1H-imidazol-1-yl)-4,5-dihydro-[1,2,4]triazolo[4,3-f]pteridine (Example 63),
(R)-5-cyclopentyl-4-ethyl-1-methyl-7-(2-methyl-1H-imidazol-1-yl)-4,5-dihydro-[1,2,4]triazolo[4,3-f]pteridine (Example 64),
(R)-5-cyclobutyl-4-ethyl-7-(2-(4-(trifluoromethyl)phenyl)-1H-imidazol-1-yl)-4,5-dihydro-[1,2,4]triazolo[4,3-f]pteridine (Example 243),
(R)-5-cyclobutyl-4-ethyl-1-methyl-7-(2-(4-(trifluoromethyl)phenyl)-1H-imidazol-1-yl)-4,5-dihydro-[1,2,4]triazolo[4,3-f]pteridine (Example 244),
(R)-5-cyclobutyl-4-ethyl-7-(2-(pyrimidin-2-yl)-1H-imidazol-1-yl)-4,5-dihydro-[1,2,4]triazolo[4,3-f]pteridine (Example 245),
(R)-5-cyclobutyl-4-ethyl-1-methyl-7-(2-(pyrimidin-2-yl)-1H-imidazol-1-yl)-4,5-dihydro-[1,2,4]triazolo[4,3-f]pteridine (Example 246),
(R)-7-(2-(3-chloro-4-fluorophenyl)-1H-imidazol-1-yl)-4-ethyl-5-isopropyl-4,5-dihydro-[1,2,4]triazolo[4,3-f]pteridine (Example 247),
(R)-7-(2-(3-chloro-4-fluorophenyl)-1H-imidazol-1-yl)-4-ethyl-5-isopropyl-1-methyl-4,5-dihydro-[1,2,4]triazolo[4,3-f]pteridine (Example 248),
14a-ethyl-7-(2-phenyl-1H-imidazol-1-yl)-10,11,12,13,14,14a-hexahydroazepino[2,1-h][1,2,4]triazolo[4,3-f]pteridine (Example 249),
14a-ethyl-3-methyl-7-(2-phenyl-1H-imidazol-1-yl)-10,11,12,13,14,14a-hexahydroazepino[2,1-h][1,2,4]triazolo[4,3-f]pteridine (Example 250),
(R)-7-(2-(3-bromophenyl)-1H-imidazol-1-yl)-4-ethyl-5-isopropyl-4,5-dihydro-[1,2,4]triazolo[4,3-f]pteridine (Example 251),
(R)-7-(2-(3-bromophenyl)-1H-imidazol-1-yl)-4-ethyl-5-isopropyl-1-methyl-4,5-dihydro-[1,2,4]triazolo[4,3-f]pteridine (Example 252),
(R)-4-(1-(4-ethyl-5-isopropyl-4,5-dihydro-[1,2,4]triazolo[4,3-f]pteridin-7-yl)-1H-imidazol-2-yl)benzonitrile (Example 253),
(R)-4-(1-(4-ethyl-5-isopropyl-1-methyl-4,5-dihydro-[1,2,4]triazolo[4,3-f]pteridin-7-yl)-1H-imidazol-2-yl)benzonitrile (Example 254),
(R)-3-(1-(4-ethyl-5-isopropyl-4,5-dihydro-[1,2,4]triazolo[4,3-f]pteridin-7-yl)-1H-imidazol-2-yl)benzonitrile (Example 255), and
(R)-3-(1-(4-ethyl-5-isopropyl-1-methyl-4,5-dihydro-[1,2,4]triazolo[4,3-f]pteridin-7-yl)-1H-imidazol-2-yl)benzonitrile (Example 256).
   The following table provides the example number (column 1), Intermediate (column 2), and ring reactant (column 3), to give the compound shown in column 4.

| Ex. | | | |
|---|---|---|---|
| No. | Int. | Ring reactant | Compound structure |
| 62 | F | 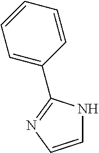 | 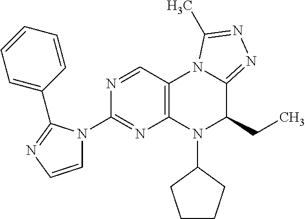 |
| 63 64 | E F | 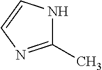 | 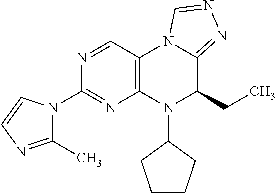 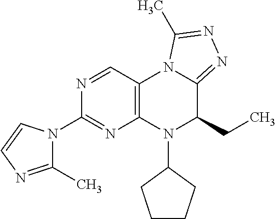 |
| 243 244 | C D | 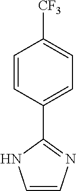 | 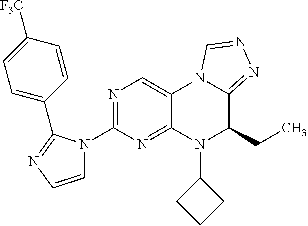 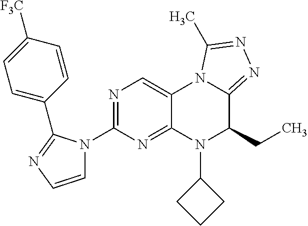 |
| 245 246 | C D | 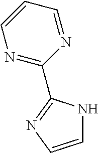 | 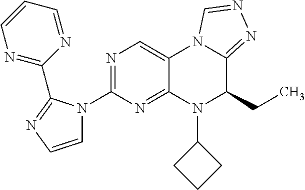 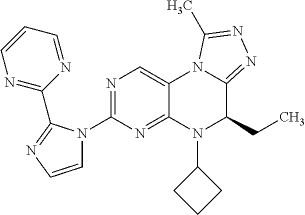 |
| 247 248 | G H | 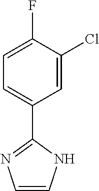 | 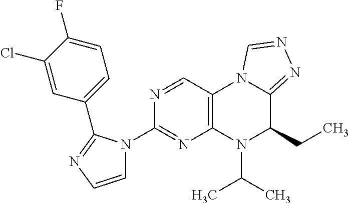 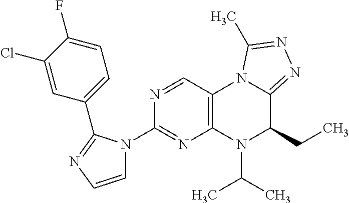 |
| 249 250 | X X′ | 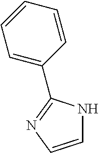 | 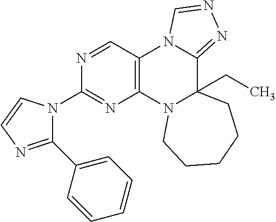 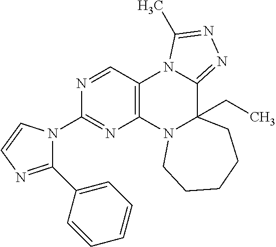 |
| 251 252 | G H | 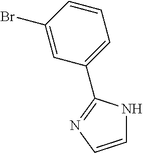 | 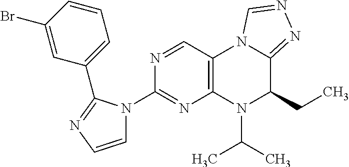 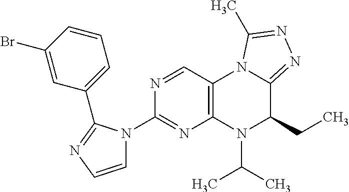 |
| 253 254 | G H | 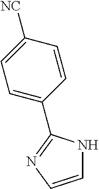 | 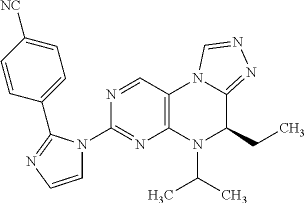 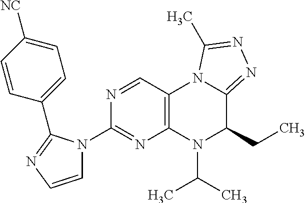 |
| 255 256 | G H | 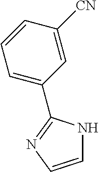 | 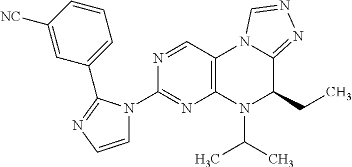 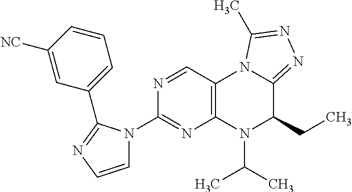 |

### Example 65

### Synthesis of (R)-1-(5-cyclopentyl-4-ethyl-4,5-dihydro-[1,2,4]triazolo[4,3-f]pteridin-7-yl)-2,3-dimethyl-1H-imidazol-3-ium

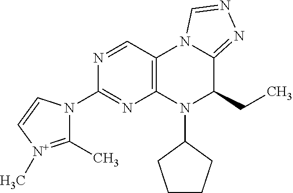

The title compound may be isolated as a side-product during the procedures of Example 63.

### Example 66

### Synthesis of (R)-1-(5-cyclopentyl-4-ethyl-1-methyl-4,5-dihydro-[1,2,4]triazolo[4,3-f]pteridin-7-yl)-2,3-dimethyl-1H-imidazol-3-ium

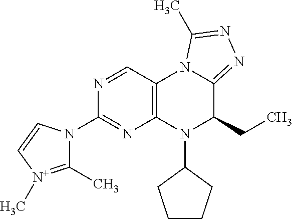

The title compound may be isolated as a side-product during the procedures of Example 64.

### Example 77

### Synthesis of (R)-2-(1-(5-cyclopentyl-4-ethyl-4,5-dihydro-[1,2,4]triazolo[4,3-f]pteridin-7-yl)-1H-imidazol-4-yl)acetonitrile

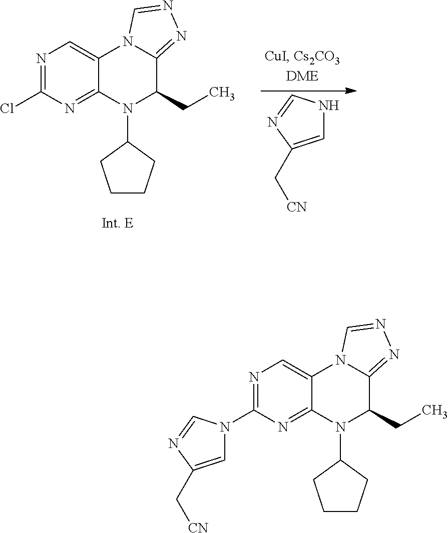

A mixture of Intermediate E, 2-(1H-imidazol-4-yl)acetonitrile (2 eq), CuI (0.1 eq), 1,2 bis(methylamino) cyclohexane and Cs₂CO₃ in DMF is purged with nitrogen and is subsequently heated in a sealed vial at 110° C. for 18 h. The reaction is diluted with ethyl acetate, filtered through Celite and evaporated. The residue is purified by reverse phase preparative HPLC and lyophilized to give the title compound.

Additional compounds are prepared similarly to this method, optionally replacing Intermediate E with a suitable intermediate, and/or replacing 2-(1H-imidazol-4-yl)acetonitrile with an appropriate ring reactant. The following compounds are prepared:
(R)-2-(1-(5-cyclopentyl-4-ethyl-1-methyl-4,5-dihydro-[1,2,4]triazolo[4,3-f]pteridin-7-yl)-1H-imidazol-4-yl)acetonitrile (Example 78),
(R)-5-cyclopentyl-4-ethyl-7-(4-phenyl-1H-imidazol-1-yl)-4,5-dihydro-[1,2,4]triazolo[4,3-f]pteridine (Example 79),
(R)-5-cyclopentyl-4-ethyl-1-methyl-7-(4-phenyl-1H-imidazol-1-yl)-4,5-dihydro-[1,2,4]triazolo[4,3-f]pteridine (Example 80),
(R)-methyl 1-(5-cyclopentyl-4-ethyl-4,5-dihydro-[1,2,4]triazolo[4,3-f]pteridin-7-yl)-1H-imidazole-4-carboxylate (Example 81),
(R)-methyl 1-(5-cyclopentyl-4-ethyl-1-methyl-4,5-dihydro-[1,2,4]triazolo[4,3-f]pteridin-7-yl)-1H-imidazole-4-carboxylate (Example 82),
(R)-7-(4-bromo-1H-imidazol-1-yl)-5-cyclopentyl-4-ethyl-4,5-dihydro-[1,2,4]triazolo[4,3-f]pteridine (Example 93),
(R)-7-(4-bromo-1H-imidazol-1-yl)-5-cyclopentyl-4-ethyl-1-methyl-4,5-dihydro-[1,2,4]triazolo[4,3-f]pteridine (Example 94),
(R)-5-cyclopentyl-4-ethyl-7-(4-methyl-1H-imidazol-1-yl)-4,5-dihydro-[1,2,4]triazolo[4,3-f]pteridine (Example 257),
(R)-5-cyclopentyl-4-ethyl-1-methyl-7-(4-methyl-1H-imidazol-1-yl)-4,5-dihydro-[1,2,4]triazolo[4,3-f]pteridine (Example 258),
(R)-1-(5-cyclopentyl-4-ethyl-4,5-dihydro-[1,2,4]triazolo[4,3-f]pteridin-7-yl)-1H-pyrazole-3-carboxylic acid (Example 259),
(R)-1-(5-cyclopentyl-4-ethyl-1-methyl-4,5-dihydro-[1,2,4]triazolo[4,3-f]pteridin-7-yl)-1H-pyrazole-3-carboxylic acid (Example 260),
(R)-5-cyclopentyl-4-ethyl-7-(4-(pyridin-3-yl)-1H-imidazol-1-yl)-4,5-dihydro-[1,2,4]triazolo[4,3-f]pteridine (Example 261),
(R)-5-cyclopentyl-4-ethyl-1-methyl-7-(4-(pyridin-3-yl)-1H-imidazol-1-yl)-4,5-dihydro-[1,2,4]triazolo[4,3-f]pteridine (Example 262),
(R)-5-cyclopentyl-4-ethyl-7-(2-(pyridin-3-yl)-1H-imidazol-1-yl)-4,5-dihydro-[1,2,4]triazolo[4,3-f]pteridine (Example 263),
(R)-5-cyclopentyl-4-ethyl-1-methyl-7-(2-(pyridin-3-yl)-1H-imidazol-1-yl)-4,5-dihydro-[1,2,4]triazolo[4,3-f]pteridine (Example 264),
(R)-5-cyclobutyl-4-ethyl-7-(1H-imidazol-1-yl)-4,5-dihydro-[1,2,4]triazolo[4,3-f]pteridine (Example 265), and
(R)-5-cyclobutyl-4-ethyl-7-(1H-imidazol-1-yl)-1-methyl-4,5-dihydro-[1,2,4]triazolo[4,3-f]pteridine (Example 266).
   The following table provides the example number (column 1), Intermediate (column 2), and ring reactant (column 3), to give the compound shown in column 4.

| Ex. | | | |
|---|---|---|---|
| No. | Int. | Ring reactant | Compound structure |
| 78 | F | 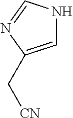 | 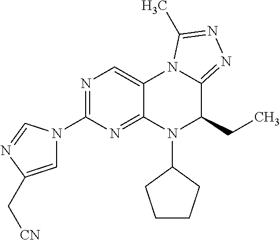 |
| 79 80 | E F | 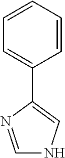 | 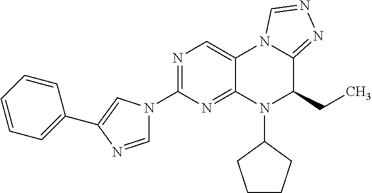 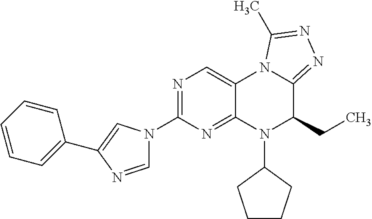 |
| 81 82 | E F | 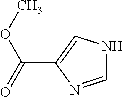 | 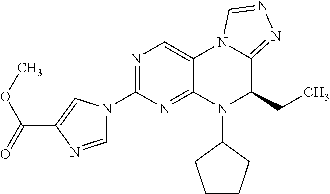 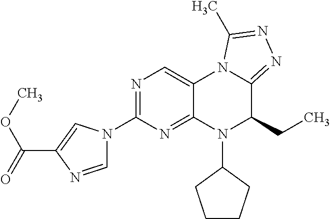 |
| 93 94 | E F | 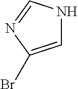 | 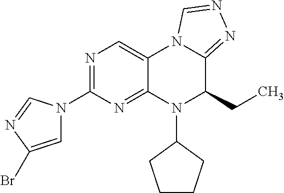 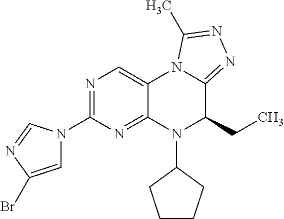 |
| 257 258 | E F | 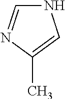 | 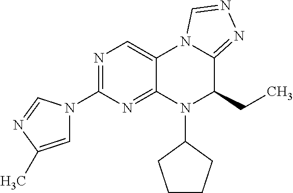 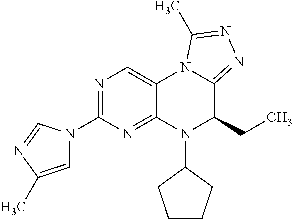 |
| 259 260 | E F | 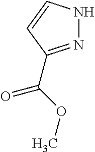 | 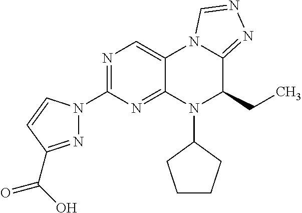 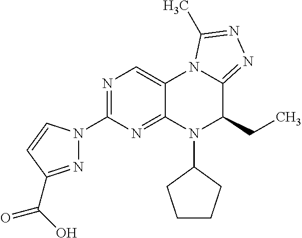 |
| 261 262 | E F | 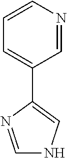 | 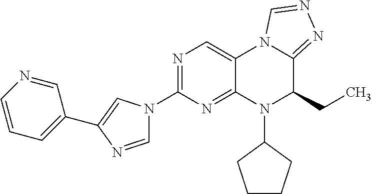 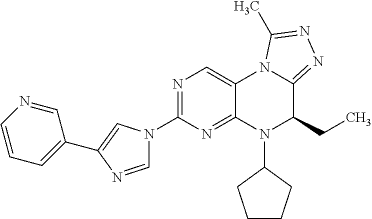 |
| 263 264 | E F | 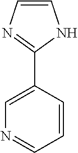 | 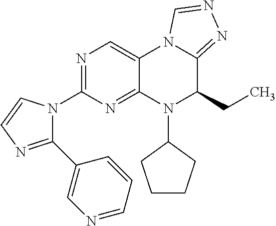 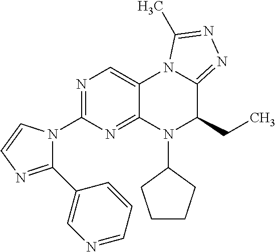 |
| 265 266 | C D | 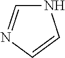 | 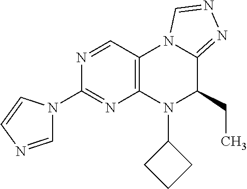 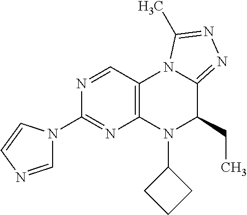 |

### Example 83

### Synthesis of (R)-1-(5-cyclopentyl-4-ethyl-4,5-dihydro-[1,2,4]triazolo[4,3-f]pteridin-7-yl)-1H-imidazole-4-carboxylic acid

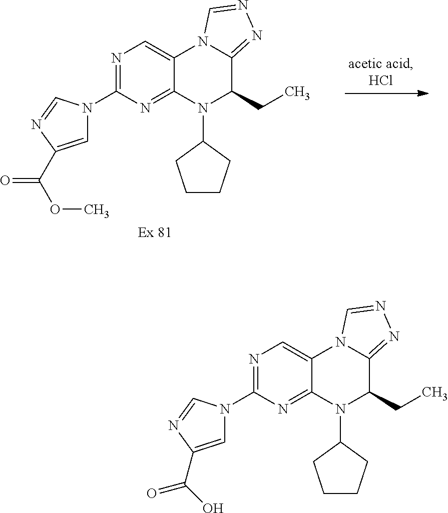

The title compound is prepared by dissolving (R)-methyl 1-(5-cyclopentyl-4-ethyl-4,5-dihydro-[1,2,4]triazolo[4,3-f]pteridin-7-yl)-1H-imidazole-4-carboxylate (Example 81) in acetic acid and concentrated aqueous HCl and heating the resulting solution to 100° C. for 4 hours. The solution is concentrated under vacuum and co-evaporated from toluene three times and the crude material is purified by preparative HPLC.

### Example 84

### Synthesis of (R)-1-(5-cyclopentyl-4-ethyl-1-methyl-4,5-dihydro-[1,2,4]triazolo[4,3-f]pteridin-7-yl)-1H-imidazole-4-carboxylic acid

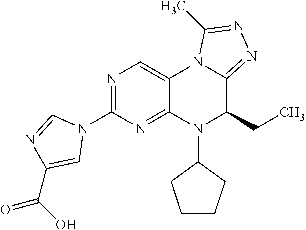

The title compound is prepared by dissolving (R)-methyl 1-(5-cyclopentyl-4-ethyl-1-methyl-4,5-dihydro-[1,2,4]triazolo[4,3-f]pteridin-7-yl)-1H-imidazole-4-carboxylate (Example 82) in acetic acid and concentrated aqueous HCl and heating the resulting solution to 100° C. for 4 hours. The solution is concentrated under vacuum and co-evaporated from toluene three times and the crude material is purified by preparative HPLC.

### Example 85

### Synthesis of (R)-1-(5-cyclopentyl-4-ethyl-4,5-dihydro-[1,2,4]triazolo[4,3-f]pteridin-7-yl)-1H-pyrazole-4-carboxylic acid

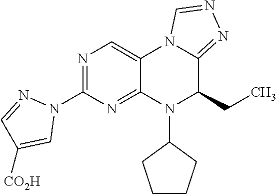

The title compound is prepared by an adaptation of the method of Example 83 using (R)-ethyl 1-(5-cyclopentyl-4-ethyl-4,5-dihydro-[1,2,4]triazolo[4,3-f]pteridin-7-yl)-1H-pyrazole-4-carboxylate (Example 45) instead of (R)-1-(5-cyclopentyl-4-ethyl-4,5-dihydro-[1,2,4]triazolo[4,3-f]pteridin-7-yl)-1H-imidazole-4-carboxylic acid.

### Example 86

### Synthesis of (R)-1-(5-cyclopentyl-4-ethyl-1-methyl-4,5-dihydro-[1,2,4]triazolo[4,3-f]pteridin-7-yl)-1H-pyrazole-4-carboxylic acid

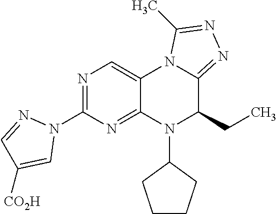

The title compound is prepared by an adaptation of the method of Example 83 using (R)-ethyl 1-(5-cyclopentyl-4-ethyl-1-methyl-4,5-dihydro-[1,2,4]triazolo[4,3-f]pteridin-7-yl)-1H-pyrazole-4-carboxylate (Example 46) instead of (R)-1-(5-cyclopentyl-4-ethyl-4,5-dihydro-[1,2,4]triazolo[4,3-f]pteridin-7-yl)-1H-imidazole-4-carboxylic acid

### Example 87

### Synthesis of (R)-1-(5-cyclopentyl-4-ethyl-4,5-dihydro-[1,2,4]triazolo[4,3-f]pteridin-7-yl)-N,N-dimethyl-1H-pyrazole-4-carboxamide

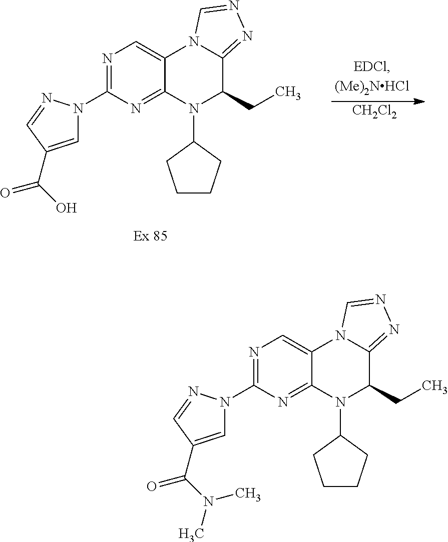

(R)-1-(5-cyclopentyl-4-ethyl-4,5-dihydro-[1,2,4]triazolo[4,3-f]pteridin-7-yl)-1H-pyrazole-4-carboxylic acid (Example 85) is dissolved in methylene chloride and EDCI (1.1 eq), dimethylamine hydrochloride (1.5 eq), HOAt (0.1 eq) and triethylamine (3 eq) are added. The resulting solution is stirred at rt for 48 hours after which the reaction mixture is diluted with methylene chloride and washed with 0.1 N aqueous HCl, then 1 N aqueous NaOH, dried (Na₂SO₄), filtered, concentrated under vacuum and purified by preparative HPLC to give the title compound.

Additional compounds are prepared similarly to this method, optionally replacing the compound of Example 85 with the compound of Example 83, 84, or 86, and/or replacing dimethylamine hydrochloride with a suitable amine reactant. The following compounds are prepared:
(R)-1-(5-cyclopentyl-4-ethyl-1-methyl-4,5-dihydro-[1,2,4]triazolo[4,3-f]pteridin-7-yl)-N,N-dimethyl-1H-pyrazole-4-carboxamide (Example 88),
(R)-1-(5-cyclopentyl-4-ethyl-4,5-dihydro-[1,2,4]triazolo[4,3-f]pteridin-7-yl)-N-methyl-1H-pyrazole-4-carboxamide (Example 89),
(R)-1-(5-cyclopentyl-4-ethyl-1-methyl-4,5-dihydro-[1,2,4]triazolo[4,3-f]pteridin-7-yl)-N-methyl-1H-pyrazole-4-carboxamide (Example 90),
(R)-(1-(5-cyclopentyl-4-ethyl-4,5-dihydro-[1,2,4]triazolo[4,3-f]pteridin-7-yl)-1H-pyrazol-4-yl)(morpholino)methanone (Example 91),
(R)-(1-(5-cyclopentyl-4-ethyl-1-methyl-4,5-dihydro-[1,2,4]triazolo[4,3-f]pteridin-7-yl)-1H-pyrazol-4-yl)(morpholino)methanone (Example 92),
(R)-(1-(5-cyclopentyl-4-ethyl-4,5-dihydro-[1,2,4]triazolo[4,3-f]pteridin-7-yl)-1H-imidazol-4-yl)(morpholino)methanone (Example 95),
(R)-(1-(5-cyclopentyl-4-ethyl-1-methyl-4,5-dihydro-[1,2,4]triazolo[4,3-f]pteridin-7-yl)-1H-imidazol-4-yl)(morpholino)methanone (Example 96),
(R)-1-(5-cyclopentyl-4-ethyl-4,5-dihydro-[1,2,4]triazolo[4,3-f]pteridin-7-yl)-N-methyl-1H-imidazole-4-carboxamide (Example 97),
(R)-1-(5-cyclopentyl-4-ethyl-1-methyl-4,5-dihydro-[1,2,4]triazolo[4,3-f]pteridin-7-yl)-N-methyl-1H-imidazole-4-carboxamide (Example 98),
(R)-1-(5-cyclopentyl-4-ethyl-4,5-dihydro-[1,2,4]triazolo[4,3-f]pteridin-7-yl)-N-methyl-1H-pyrazole-3-carboxamide (Example 267),
(R)-1-(5-cyclopentyl-4-ethyl-1-methyl-4,5-dihydro-[1,2,4]triazolo[4,3-f]pteridin-7-yl)-N-methyl-1H-pyrazole-3-carboxamide (Example 268),
(R)-1-(5-cyclopentyl-4-ethyl-4,5-dihydro-[1,2,4]triazolo[4,3-f]pteridin-7-yl)-N,N-dimethyl-1H-pyrazole-3-carboxamide (Example 269),
(R)-1-(5-cyclopentyl-4-ethyl-1-methyl-4,5-dihydro-[1,2,4]triazolo[4,3-f]pteridin-7-yl)-N,N-dimethyl-1H-pyrazole-3-carboxamide (Example 270),
(R)-1-(5-cyclopentyl-4-ethyl-4,5-dihydro-[1,2,4]triazolo[4,3-f]pteridin-7-yl)-1H-pyrazole-3-carboxamide (Example 271),
(R)-1-(5-cyclopentyl-4-ethyl-1-methyl-4,5-dihydro-[1,2,4]triazolo[4,3-f]pteridin-7-yl)-1H-pyrazole-3-carboxamide (Example 272),
(R)-1-(5-cyclopentyl-4-ethyl-4,5-dihydro-[1,2,4]triazolo[4,3-f]pteridin-7-yl)-1H-imidazole-4-carboxamide (Example 273), and
(R)-1-(5-cyclopentyl-4-ethyl-1-methyl-4,5-dihydro-[1,2,4]triazolo[4,3-f]pteridin-7-yl)-1H-imidazole-4-carboxamide (Example 274),
(R)-1-(5-cyclopentyl-4-ethyl-4,5-dihydro-[1,2,4]triazolo[4,3-f]pteridin-7-yl)-N,N-dimethyl-1H-imidazole-4-carboxamide (Example 687), and
(R)-1-(5-cyclopentyl-4-ethyl-1-methyl-4,5-dihydro-[1,2,4]triazolo[4,3-f]pteridin-7-yl)-N,N-dimethyl-1H-imidazole-4-carboxamide (Example 688).
   The following table provides the example number (column 1), starting compound (SC) Example number (column 2), and amine reactant (column 3), to give the compound shown in column 4.

| Ex. | | | |
|---|---|---|---|
| No. | SC | Amine reactant | Compound structure |
| 88 | 86 | 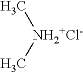 | 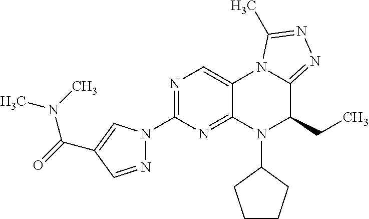 |
| 89 90 | 85 86 | 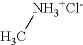 | 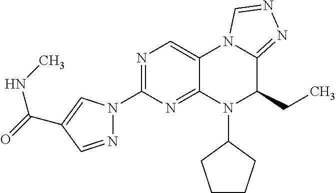 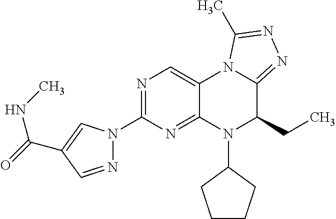 |
| 91 92 | 85 86 | 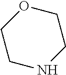 | 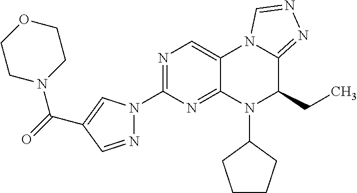 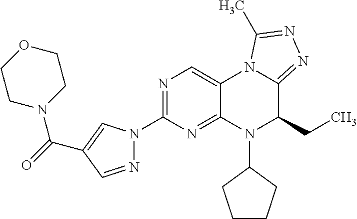 |
| 95 96 | 83 84 | 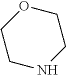 | 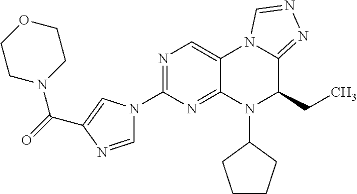 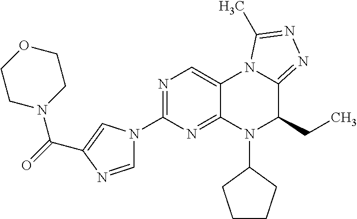 |
| 97 98 | 83 84 | 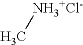 | 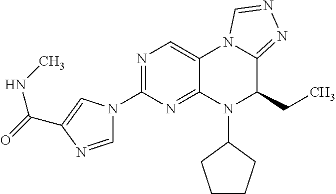 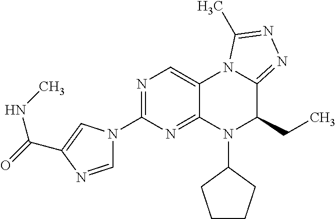 |
| 267 268 | 259 260 | 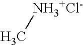 | 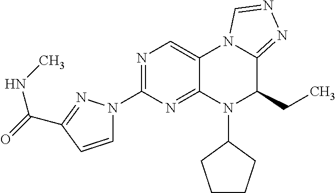 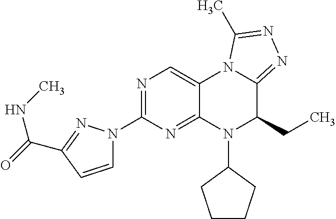 |
| 269 270 | 259 260 | 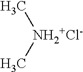 | 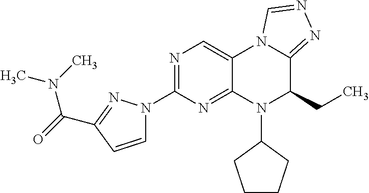 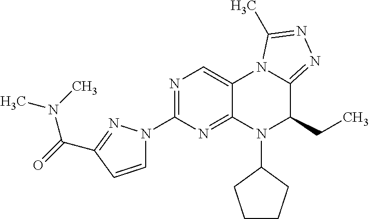 |
| 271 272 | 259 260 | NH₄OAc | 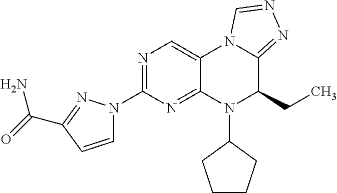 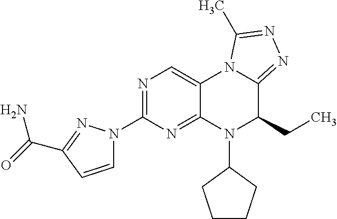 |
| 273 274 | 83 84 | NH4OAc | 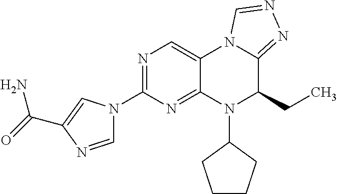 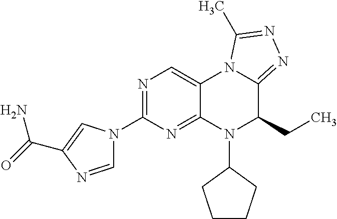 |
| 687 688 | 83 84 | 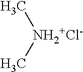 | 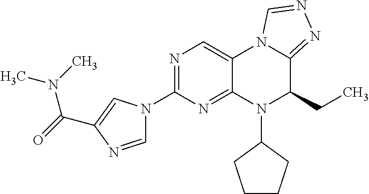 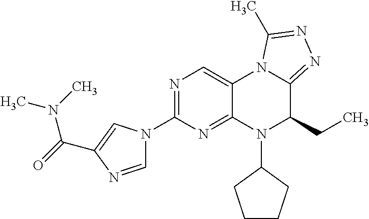 |

### Example 275 and Example 276

### Synthesis of (R)-7-(2-bromo-1H-imidazol-1-yl)-5-cyclopentyl-4-ethyl-4,5-dihydro-[1,2,4]triazolo[4,3-f]pteridine (275) and (R)-7-(2-bromo-1H-imidazol-1-yl)-5-cyclopentyl-4-ethyl-1-methyl-4,5-dihydro-[1,2,4]triazolo[4,3-f]pteridine (276)

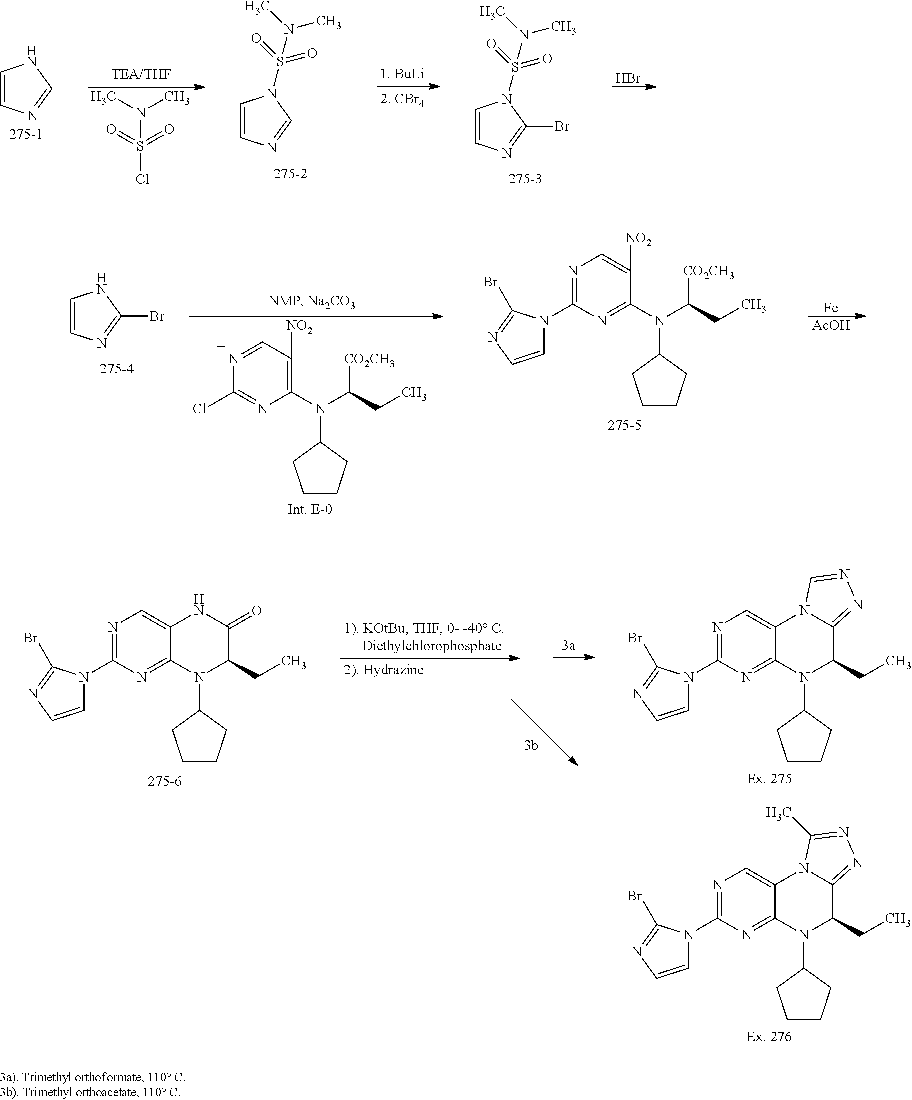

1H-imidazole (275-1, 10 g) was dissolved in 150 mL of THF with dimethylsulfamoyl chloride (19 g), followed by the drop-wise addition of TEA (20 g). The mixture was stirred at rt for 16 h, then poured into 200 mL of water and extracted with EtOAc. The organic layer was dried with Na₂SO₄. Solvent was removed to give compound 275-2 as a light yellow oil.

Compound 275-2 (1.5 g) was dissolved in 20 mL of THF and cooled to −78° C. and n-BuLi (4.1 ml, 2.5 M in hexanes) was added drop-wise at −78° C., then CBr₄ (1.1 eq) was added and the mixture was stirred at rt for 16 h. Forty mL of water was added and the suspension was extracted with EtOAc and dried with Na₂SO₄. The solvent was removed and the residue was purified with silica column (PE: DCM) to give compound 275-3.

Compound 275-3 (1.1 g) was placed in a 50 ml round flask and HBr (40%, 10 ml in water) was added to give a suspension. The mixture was stirred at rt for 16 h to give a deep yellow solution, then the pH was adjusted to 8 and the mixture was extracted with EtOAc. The solvent was removed to give compound 275-4 as a yellow solid.

Intermediate E-0 (13.6 g) was dissolved in 80 mL of NMP and compound 275-4 (6.5 g) and Na₂CO₃ (4.6 g) were added. The solution was stirred at 90° C. for 6 h, then NMP was removed under reduced pressure. The residue was dissolved in EtOAc, washed with water and purified by silica gel flash chromatography (PE: EA=2:1) to give compound 275-5 as a yellow oil.

Compound 275-5 (13.7 g) was dissolved in 150 mL of AcOH, iron powder (20 g) was added and the mixture was stirred at 42° C. for 40 min. The cooled solution was added carefully to aq. Na₂CO₃ and extracted with EtOAc, then purified by flash chromatography (DCM: EA=85:15 then 1:1) to give compound 275-6.

A solution of compound 275-6 in THF is stirred at −20° C. and potassium tert-butoxide (1.3 eq) is added over 5 min. The reaction mixture is warmed up to 0° C. for 25 min after complete addition. The reaction mixture is cooled to −40° C. and diethylchlorophosphate (1.4 eq) is added. The reaction mixture is warmed up to rt for 45 min. To the resulting mixture, 1M hydrazine (10 eq) is added and the reaction mixture is stirred at rt for 18 h. The reaction mixture is concentrated under reduced pressure and diluted with DCM and a saturated NaHCO₃ solution. The organic layer is dried over MgSO₄ and concentrated under pressure. The resulting material is purified by MPLC, then dissolved in trimethyl orthoformate (10 eq) or trimethyl orthoacetate (10 eq) and heated to 110° C. for 1 h. The reaction mixture is concentrated under reduced pressure and purified via silica gel column chromatography to give Example 275 (from orthoformate reaction) or Example 276 (from orthoacetate reaction).

### Example 277

### Synthesis of (R)-4-(1-(5-cyclopentyl-4-ethyl-4,5-dihydro-[1,2,4]triazolo[4,3-f]pteridin-7-yl)-1H-imidazol-4-yl)thiazole

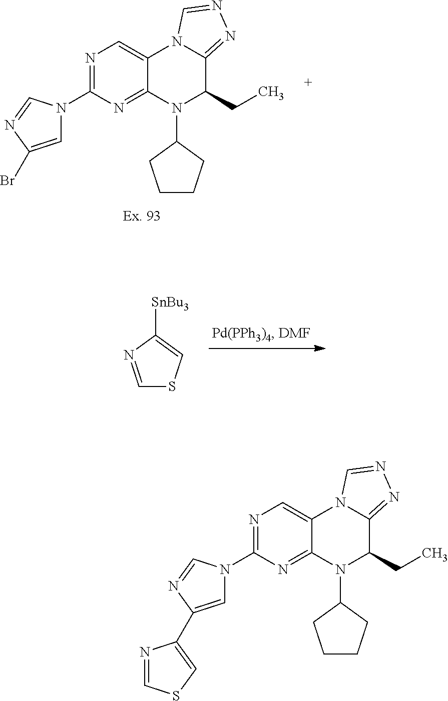

(R)-7-(4-bromo-1H-imidazol-1-yl)-5-cyclopentyl-4-ethyl-4,5-dihydro-[1,2,4]triazolo[4,3-f]pteridine (Example 93), 4-(tributylstannyl)thiazole (1 eq, see Example 693) and Pd(PPh₃)₄ (0.1 eq) are dissolved in DMF in a screw cap vial and a stream of nitrogen is bubbled through the mixture for 2 minutes. The vial is sealed and the resulting solution is stirred at 100° C. for 19 h. The reaction mixture is diluted with brine, extracted with EtOAc, dried with Na₂SO₄ then purified by flash chromatography with a silica gel column by eluting with a mixture of Hexane:EtOAc and then further purified by preparative HPLC to give the title compound.

Additional compounds are prepared similarly to this method, optionally replacing the compound of Example 93 with a suitable compound, and/or replacing 4-(tributylstannyl)thiazole with a suitable tributylstannyl derivative compound (which can be prepared similarly to methods of Example 693). The following compounds are prepared:
(R)-4-(1-(5-cyclopentyl-4-ethyl-1-methyl-4,5-dihydro-[1,2,4]triazolo[4,3-f]pteridin-7-yl)-1H-imidazol-4-yl)thiazole (Example 278),
(R)-2-(1-(5-cyclopentyl-4-ethyl-4,5-dihydro-[1,2,4]triazolo[4,3-f]pteridin-7-yl)-1H-imidazol-4-yl)thiazole (Example 279),
(R)-2-(1-(5-cyclopentyl-4-ethyl-1-methyl-4,5-dihydro-[1,2,4]triazolo[4,3-f]pteridin-7-yl)-1H-imidazol-4-yl)thiazole (Example 280),
(R)-4-(1-(5-cyclopentyl-4-ethyl-4,5-dihydro-[1,2,4]triazolo[4,3-f]pteridin-7-yl)-1H-imidazol-2-yl)thiazole (Example 281),
(R)-4-(1-(5-cyclopentyl-4-ethyl-1-methyl-4,5-dihydro-[1,2,4]triazolo[4,3-f]pteridin-7-yl)-1H-imidazol-2-yl)thiazole (Example 282),
(R)-2-(1-(5-cyclopentyl-4-ethyl-4,5-dihydro-[1,2,4]triazolo[4,3-f]pteridin-7-yl)-1H-imidazol-2-yl)thiazole (Example 283),
(R)-2-(1-(5-cyclopentyl-4-ethyl-1-methyl-4,5-dihydro-[1,2,4]triazolo[4,3-f]pteridin-7-yl)-1H-imidazol-2-yl)thiazole (Example 284),
(R)-2-(1-(5-cyclopentyl-4-ethyl-4,5-dihydro-[1,2,4]triazolo[4,3-f]pteridin-7-yl)-1H-imidazol-2-yl)oxazole (Example 285), and
(R)-2-(1-(5-cyclopentyl-4-ethyl-1-methyl-4,5-dihydro-[1,2,4]triazolo[4,3-f]pteridin-7-yl)-1H-imidazol-2-yl)oxazole (Example 286).
   The following table provides the example number (column 1), starting compound (SC) Example number (column 2), and tributylstannyl reactant (column 3), to give the compound shown in column 4.

| | | t-Butylstannyl | |
|---|---|---|---|
| Ex. No. | SC | reactant | Compound structure |
| 278 | 94 | 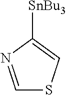 | 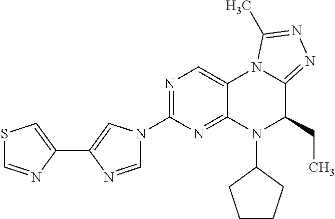 |
| 279 | 93 | 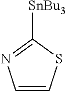 | 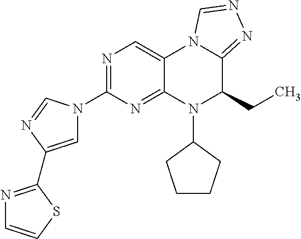 |
| 280 | 94 | | 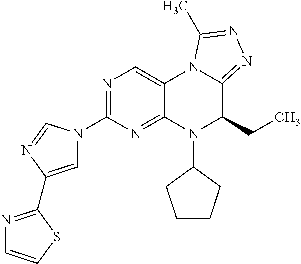 |
| 281 | 275 | 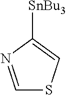 | 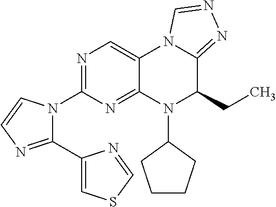 |
| 282 | 276 | | 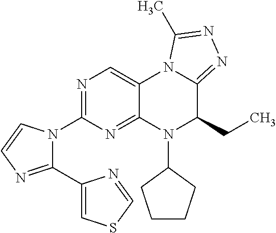 |
| 283 | 275 | 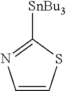 | 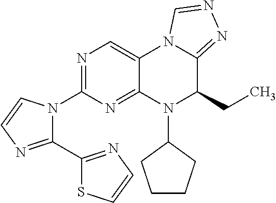 |
| 284 | 276 | | 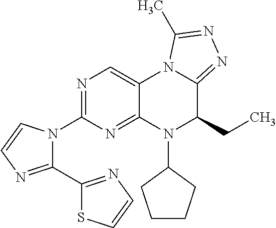 |
| 285 | 275 | 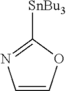 | 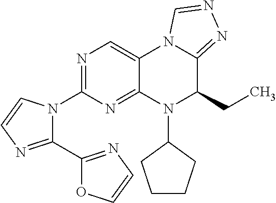 |
| 286 | 276 | | 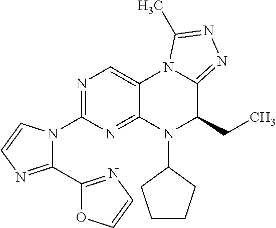 |

### Example 287 and Example 288

### Synthesis of (R)-2-(1-(5-cyclopentyl-4-ethyl-4,5-dihydro-[1,2,4]triazolo[4,3-f]pteridin-7-yl)-1H-imidazol-4-yl)ethanamine (287) and (R)-2-(1-(5-cyclopentyl-4-ethyl-1-methyl-4,5-dihydro-[1,2,4]triazolo[4,3-f]pteridin-7-yl)-1H-imidazol-4-yl)ethanamine (288)

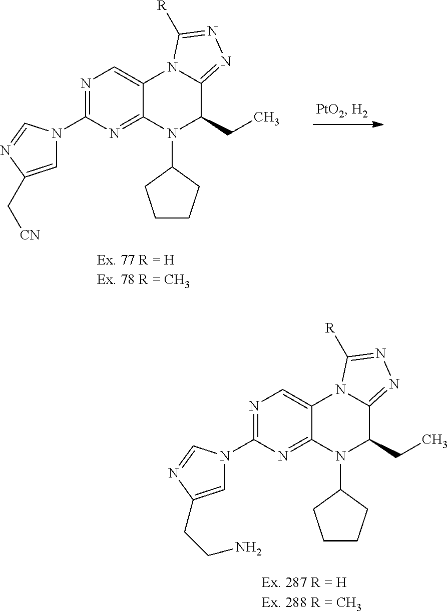

(R)-2-(1-(5-cyclopentyl-4-ethyl-4,5-dihydro-[1,2,4]triazolo[4,3-f]pteridin-7-yl)-1H-imidazol-4-yl)acetonitrile or (R)-2-(1-(5-cyclopentyl-4-ethyl-1-methyl-4,5-dihydro-[1,2,4]triazolo[4,3-f]pteridin-7-yl)-1H-imidazol-4-yl)acetonitrile (Example 77 or Example 78, 0.218 mmol) and PtO₂ (40 mg) are suspended in 2 mL of EtOAc and the resulting mixture is stirred under an atmosphere of hydrogen (1 atm, balloon) for 18 h. The resulting solution is filtered through Celite, concentrated, then purified by preparative HPLC to give the title compounds.

### Example 289 and Example 290

### Synthesis of (R)-1-(5-cyclopentyl-4-ethyl-4,5-dihydro-[1,2,4]triazolo[4,3-f]pteridin-7-yl)pyridin-4(1H)-imine (289) and (R)-1-(5-cyclopentyl-4-ethyl-1-methyl-4,5-dihydro-[1,2,4] triazolo[4,3-f]pteridin-7-yl)pyridin-4(1H)-imine (290)

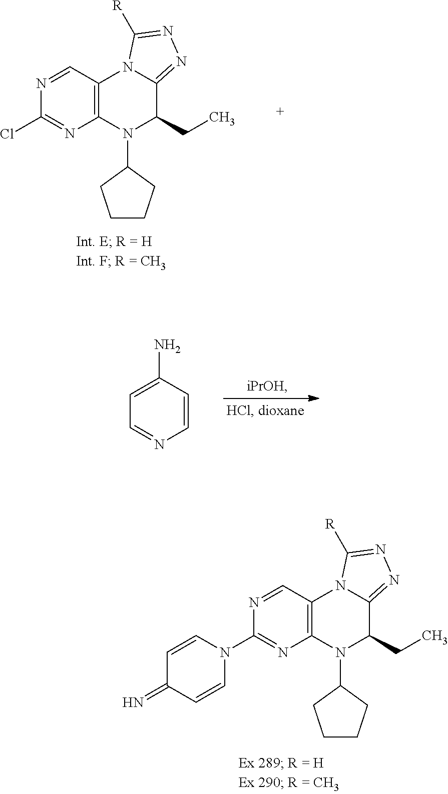

To a solution of the Intermediate E or F (1.36 mmol) in 5 mL of isopropanol in a microwave vial, 4N HCl in dioxane (0.43 mL) and 4-aminopyridine (2 eq) are added and the vial is heated in a microwave oven at 160° C. for 1 hour. Solvent is removed under reduced pressure and the resulting yellow solid is purified by reversed phase HPLC to give the title compounds.

### Example 291 and Example 292

### Synthesis of (R)-7-(2-benzyl-1H-imidazol-1-yl)-5-cyclopentyl-4-ethyl-4,5-dihydro-[1,2,4]triazolo[4,3-f]pteridine (291) and (R)-7-(2-benzyl-1H-imidazol-1-yl)-5-cyclopentyl-4-ethyl-1-methyl-4,5-dihydro-[1,2,4]triazolo[4,3-f]pteridine (292)

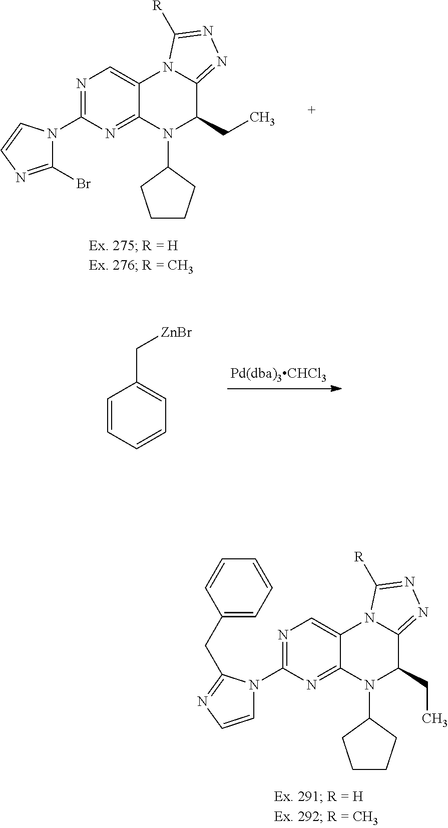

(R)-7-(2-bromo-1H-imidazol-1-yl)-5-cyclopentyl-4-ethyl-4,5-dihydro-[1,2,4]triazolo[4,3-f]pteridine or (R)-7-(2-bromo-1H-imidazol-1-yl)-5-cyclopentyl-4-ethyl-1-methyl-4,5-dihydro-[1,2,4]triazolo[4,3-f]pteridine (Example 275 or 276, 0.518 mmol), Pd₂(dba)₃.CHCl₃ (0.0518 mmol) and biphenyl-2-yldi-tert-butylphosphine (0.103 mmol) are placed in a screw cap vial and a solution of benzyl zinc bromide (1.5 mL, 0.777 mmol in THF) is added. A stream of nitrogen is bubbled through the mixture for 2 minutes and then the vial is sealed and the resulting solution is stirred at 90° C. for 18 h. The reaction mixture is filtered, then purified by flash chromatography with a silica gel column by eluting with a mixture of Hexane:EtOAc and then further purified by preparative HPLC to give the title compounds.

### Example 293

### Synthesis of (R)-4-ethyl-5-isopropyl-7-(2-phenyl-1H-imidazol-1-yl)-4,5-dihydro-[1,2,4]triazolo[4,3-f]pteridine

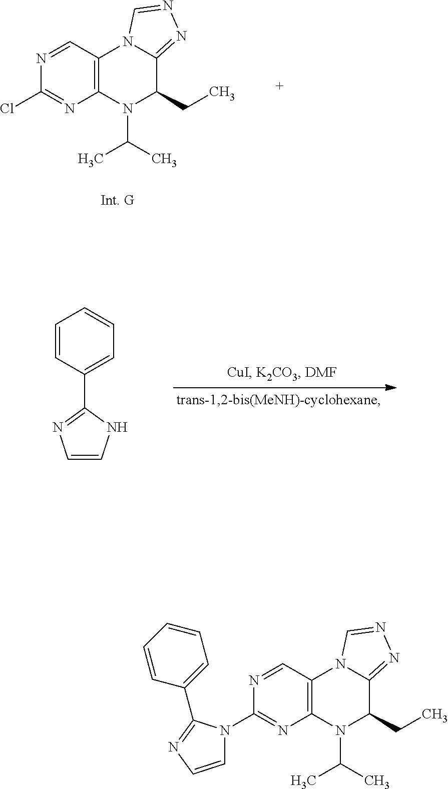

A mixture of Intermediate G (0.37 mmol), 2-phenyl-1H-imidazole (3.7 mmol), CuI (0.18 mmol), trans-1,2-bis(methylamino)cyclo-hexane (0.37 mmol) and solid K₂CO₃ (511 mg, 3.7 mmol) in 2 mL of DMF is heated in a microwave reaction apparatus for 2 h at 200° C. After this time the reaction is transferred to a round bottom flask with the aid of EtOAc, then evaporated. The residue is purified by reverse-phase HPLC (PLRPS C-18 column, eluting with a gradient of 20-25% acetonitrile in water over 30 min) to give the title compound.

Additional compounds are prepared similarly to this method, optionally replacing Intermediate G with a suitable Intermediate, and/or 2-phenyl-1H-imidazole with a suitable ring reactant. The following compounds are prepared:
(R)-4-ethyl-5-isopropyl-1-methyl-7-(2-phenyl-1H-imidazol-1-yl)-4,5-dihydro-[1,2,4]triazolo[4,3-f]pteridine (Example 294),
(R)-5-cyclobutyl-4-ethyl-7-(2-(pyridin-4-yl)-1H-imidazol-1-yl)-4,5-dihydro-[1,2,4]triazolo[4,3-f]pteridine (Example 295),
(R)-5-cyclobutyl-4-ethyl-1-methyl-7-(2-(pyridin-4-yl)-1H-imidazol-1-yl)-4,5-dihydro-[1,2,4]triazolo[4,3-f]pteridine (Example 296),
(R)-4-ethyl-7-(2-(pyridin-4-yl)-1H-imidazol-1-yl)-5-(tetrahydro-2H-pyran-4-yl)-4,5-dihydro-[1,2,4]triazolo[4,3-f]pteridine (Example 297),
(R)-4-ethyl-1-methyl-7-(2-(pyridin-4-yl)-1H-imidazol-1-yl)-5-(tetrahydro-2H-pyran-4-yl)-4,5-dihydro-[1,2,4]triazolo[4,3-f]pteridine (Example 298),
(R)-4-ethyl-7-(2-(pyrazin-2-yl)-1H-imidazol-1-yl)-5-(tetrahydro-2H-pyran-4-yl)-4,5-dihydro-[1,2,4]triazolo[4,3-f]pteridine (Example 299),
(R)-4-ethyl-1-methyl-7-(2-(pyrazin-2-yl)-1H-imidazol-1-yl)-5-(tetrahydro-2H-pyran-4-yl)-4,5-dihydro-[1,2,4]triazolo[4,3-f]pteridine (Example 300),
(R)-5-cyclopropyl-4-ethyl-1-7-(2-(pyrazin-2-yl)-1H-imidazol-1-yl)-4,5-dihydro-[1,2,4]triazolo[4,3-f]pteridine (Example 301),
(R)-5-cyclopropyl-4-ethyl-1-methyl-7-(2-(pyrazin-2-yl)-1H-imidazol-1-yl)-4,5-dihydro-[1,2,4]triazolo[4,3-f]pteridine (Example 302),
(R)-5-cyclopropyl-4-ethyl-7-(2-(pyrimidin-5-yl)-1H-imidazol-1-yl)-4,5-dihydro-[1,2,4]triazolo[4,3-f]pteridine (Example 303),
(R)-5-cyclopropyl-4-ethyl-1-methyl-7-(2-(pyrimidin-5-yl)-1H-imidazol-1-yl)-4,5-dihydro-[1,2,4]triazolo[4,3-f]pteridine (Example 304),
(R)-5-cyclopropyl-4-ethyl-7-(2-(pyridin-4-yl)-1H-imidazol-1-yl)-4,5-dihydro-[1,2,4]triazolo[4,3-f]pteridine (Example 305),
(R)-5-cyclopropyl-4-ethyl-1-methyl-7-(2-(pyridin-4-yl)-1H-imidazol-1-yl)-4,5-dihydro-[1,2,4]triazolo[4,3-f]pteridine (Example 306),
(R)-4-ethyl-5-isopropyl-7-(2-(pyrimidin-2-yl)-1H-imidazol-1-yl)-4,5-dihydro-[1,2,4]triazolo[4,3-f]pteridine (Example 307),
(R)-4-ethyl-5-isopropyl-1-methyl-7-(2-(pyrimidin-2-yl)-1H-imidazol-1-yl)-4,5-dihydro-[1,2,4]triazolo[4,3-f]pteridine (Example 308),
(R)-4-ethyl-5-isopropyl-7-(2-(pyrimidin-5-yl)-1H-imidazol-1-yl)-4,5-dihydro-[1,2,4]triazolo[4,3-f]pteridine (Example 309),
(R)-4-ethyl-5-isopropyl-1-methyl-7-(2-(pyrimidin-5-yl)-1H-imidazol-1-yl)-4,5-dihydro-[1,2,4]triazolo[4,3-f]pteridine (Example 310),
(R)-4-ethyl-5-isopropyl-7-(2-(pyridin-4-yl)-1H-imidazol-1-yl)-4,5-dihydro-[1,2,4]triazolo[4,3-f]pteridine (Example 311),
(R)-4-ethyl-5-isopropyl-1-methyl-7-(2-(pyridin-4-yl)-1H-imidazol-1-yl)-4,5-dihydro-[1,2,4]triazolo[4,3-f]pteridine (Example 312),
(R)-2-(1-(4-ethyl-5-isopropyl-4,5-dihydro-[1,2,4]triazolo[4,3-f]pteridin-7-yl)-1H-imidazol-2-yl)thiazole (Example 313),
(R)-2-(1-(4-ethyl-5-isopropyl-1-methyl-4,5-dihydro-[1,2,4]triazolo[4,3-f]pteridin-7-yl)-1H-imidazol-2-yl)thiazole (Example 314),
(R)-4-ethyl-5-isopropyl-7-(2-(pyridazin-3-yl)-1H-imidazol-1-yl)-4,5-dihydro-[1,2,4]triazolo[4,3-f]pteridine (Example 315),
(R)-4-ethyl-5-isopropyl-1-methyl-7-(2-(pyridazin-3-yl)-1H-imidazol-1-yl)-4,5-dihydro-[1,2,4]triazolo[4,3-f]pteridine (Example 316),
(R)-4-ethyl-5-isopropyl-7-(2-(pyridin-2-yl)-1H-imidazol-1-yl)-4,5-dihydro-[1,2,4]triazolo[4,3-f]pteridine (Example 317),
(R)-4-ethyl-5-isopropyl-1-methyl-7-(2-(pyridin-2-yl)-1H-imidazol-1-yl)-4,5-dihydro-[1,2,4]triazolo[4,3-f]pteridine (Example 318),
(R)-4-ethyl-5-isopropyl-7-(2-(pyridin-3-yl)-1H-imidazol-1-yl)-4,5-dihydro-[1,2,4]triazolo[4,3-f]pteridine (Example 319),
(R)-4-ethyl-5-isopropyl-1-methyl-7-(2-(pyridin-3-yl)-1H-imidazol-1-yl)-4,5-dihydro-[1,2,4]triazolo[4,3-f]pteridine (Example 320),
4-perdeuteroethyl-5-perdeuteroisopropyl-7-(2-phenyl-1H-imidazol-1-yl)-4,5-dihydro-[1,2,4]triazolo[4,3-f]pteridine (Example 321),
4-perdeuteroethyl-5-perdeuteroisopropyl-1-methyl-7-(2-phenyl-1H-imidazol-1-yl)-4,5-dihydro-[1,2,4]triazolo[4,3-f]pteridine (Example 322),
(R)-2-(1-(5-cyclobutyl-4-ethyl-4,5-dihydro-[1,2,4]triazolo[4,3-f]pteridin-7-yl)-1H-imidazol-2-yl)thiazole (Example 323),
(R)-2-(1-(5-cyclobutyl-4-ethyl-1-methyl-4,5-dihydro-[1,2,4]triazolo[4,3-f]pteridin-7-yl)-1H-imidazol-2-yl)thiazole (Example 324),
(R)-2-(1-(4-ethyl-5-(tetrahydro-2H-pyran-4-yl)-4,5-dihydro-[1,2,4]triazolo[4,3-f]pteridin-7-yl)-1H-imidazol-2-yl)thiazole (Example 325),
(R)-2-(1-(4-ethyl-1-methyl-5-(tetrahydro-2H-pyran-4-yl)-4,5-dihydro-[1,2,4]triazolo[4,3-f]pteridin-7-yl)-1H-imidazol-2-yl)thiazole (Example 326),
(R)-2-(1-(4-perdeuteroethyl-5-perdeuteroisopropyl-4,5-dihydro-[1,2,4]triazolo[4,3-f]pteridin-7-yl)-1H-imidazol-2-yl)thiazole (Example 327),
(R)-2-(1-(4-perdeuteroethyl-5-perdeuteroisopropyl-1-methyl-4,5-dihydro-[1,2,4]triazolo[4,3-f]pteridin-7-yl)-1H-imidazol-2-yl)thiazole (Example 328),
(R)-5-cyclopentyl-4-ethyl-7-(2-(pyrimidin-5-yl)-1H-imidazol-1-yl)-4,5-dihydro-[1,2,4]triazolo[4,3-f]pteridine (Example 329),
(R)-5-cyclopentyl-4-ethyl-1-methyl-7-(2-(pyrimidin-5-yl)-1H-imidazol-1-yl)-4,5-dihydro-[1,2,4]triazolo[4,3-f]pteridine (Example 330),
(R)-4-ethyl-5-isopropyl-7-(2-(4-(trifluoromethyl)phenyl)-1H-imidazol-1-yl)-4,5-dihydro-[1,2,4]triazolo[4,3-f]pteridine (Example 331),
(R)-4-ethyl-5-isopropyl-1-methyl-7-(2-(4-(trifluoromethyl)phenyl)-1H-imidazol-1-yl)-4,5-dihydro-[1,2,4]triazolo[4,3-f]pteridine (Example 332),
(R)-5-cyclobutyl-4-ethyl-7-(2-(3-fluoro-5-(trifluoromethyl)phenyl)-1H-imidazol-1-yl)-4,5-dihydro-[1,2,4]triazolo[4,3-f]pteridine (Example 333),
(R)-5-cyclobutyl-4-ethyl-7-(2-(3-fluoro-5-(trifluoromethyl)phenyl)-1H-imidazol-1-yl)-1-methyl-4,5-dihydro-[1,2,4]triazolo[4,3-f]pteridine (Example 334),
(R)-5-cyclobutyl-4-ethyl-7-(2-(pyrimidin-5-yl)-1H-imidazol-1-yl)-4,5-dihydro-[1,2,4]triazolo[4,3-f]pteridine (Example 335),
(R)-5-cyclobutyl-4-ethyl-1-methyl-7-(2-(pyrimidin-5-yl)-1H-imidazol-1-yl)-4,5-dihydro-[1,2,4]triazolo[4,3-f]pteridine (Example 336),
(R)-5-(3,3-difluorocyclobutyl)-4-ethyl-7-(2-(3-fluorophenyl)-1H-imidazol-1-yl)-4,5-dihydro-[1,2,4]triazolo[4,3-f]pteridine (Example 337),
(R)-5-(3,3-difluorocyclobutyl)-4-ethyl-7-(2-(3-fluorophenyl)-1H-imidazol-1-yl)-1-methyl-4,5-dihydro-[1,2,4]triazolo[4,3-f]pteridine (Example 338),
(R)-4-ethyl-5-isopropyl-7-(2-(isoquinolin-1-yl)-1H-imidazol-1-yl)-4,5-dihydro-[1,2,4]triazolo[4,3-f]pteridine (Example 339),
(R)-4-ethyl-5-isopropyl-7-(2-(isoquinolin-1-yl)-1H-imidazol-1-yl)-1-methyl-4,5-dihydro-[1,2,4]triazolo[4,3-f]pteridine (Example 340),
(R)-7-(2-(3-chlorophenyl)-1H-imidazol-1-yl)-4-ethyl-5-isopropyl-4,5-dihydro-[1,2,4]triazolo[4,3-f]pteridine (Example 341),
(R)-7-(2-(3-chlorophenyl)-1H-imidazol-1-yl)-4-ethyl-5-isopropyl-1-methyl-4,5-dihydro-[1,2,4]triazolo[4,3-f]pteridine (Example 342),
(R)-4-ethyl-7-(2-(4-fluorophenyl)-1H-imidazol-1-yl)-5-isopropyl-4,5-dihydro-[1,2,4]triazolo[4,3-f]pteridine (Example 343),
(R)-4-ethyl-7-(2-(4-fluorophenyl)-1H-imidazol-1-yl)-5-isopropyl-1-methyl-4,5-dihydro-[1,2,4]triazolo[4,3-f]pteridine (Example 344),
(R)- 5-cyclobutyl-4-ethyl-7-(2-(5-fluoropyridin-2-yl)-1H-imidazol-1-yl)-4,5-dihydro-[1,2,4]triazolo[4,3-f]pteridine (Example 345),
(R)-5-cyclobutyl-4-ethyl-7-(2-(5-fluoropyridin-2-yl)-1H-imidazol-1-yl)-1-methyl-4,5-dihydro-[1,2,4]triazolo[4,3-f]pteridine (Example 346),
(R)-7-(2-(3,4-difluorophenyl)-1H-imidazol-1-yl)-4-ethyl-5-(3-fluorocyclobutyl)-4,5-dihydro-[1,2,4]triazolo[4,3-f]pteridine (Example 347),
(R)-7-(2-(3,4-difluorophenyl)-1H-imidazol-1-yl)-4-ethyl-5-(3-fluorocyclobutyl)-1-methyl-4,5-dihydro-[1,2,4]triazolo[4,3-f]pteridine (Example 348),
(R)-5-cyclopentyl-4-ethyl-7-(2-(5-fluoropyridin-2-yl)-1H-imidazol-1-yl)-4,5-dihydro-[1,2,4]triazolo[4,3-f]pteridine (Example 349),
(R)-5-cyclopentyl-4-ethyl-7-(2-(5-fluoropyridin-2-yl)-1H-imidazol-1-yl)-1-methyl-4,5-dihydro-[1,2,4]triazolo[4,3-f]pteridine (Example 350),
4-ethyl-7-(2-(4-fluorophenyl)-1H-imidazol-1-yl)-4-methyl-5-(3,3,3-trifluoropropyl)-4,5-dihydro-[1,2,4]triazolo[4,3-f]pteridine (Example 351),
4-ethyl-7-(2-(4-fluorophenyl)-1H-imidazol-1-yl)-1,4-dimethyl-5-(3,3,3-trifluoropropyl)-4,5-dihydro-[1,2,4]triazolo[4,3-f]pteridine (Example 352),
(R)-5-cyclopentyl-4-ethyl-7-(1H-indol-1-yl)-4,5-dihydro-[1,2,4]triazolo[4,3-f]pteridine (Example 353), and
(R)-5-cyclopentyl-4-ethyl-7-(1H-indol-1-yl)-1-methyl-4,5-dihydro-[1,2,4]triazolo[4,3-f]pteridine (Example 354).
   The following table provides the example number (column 1), Intermediate (column 2), and ring reactant (column 3), to give the compound shown in column 4.

| Ex. | | | |
|---|---|---|---|
| No. | Int. | ring reactant | Compound structure |
| 294 | H | 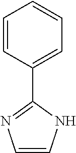 | 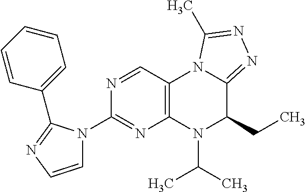 |
| 295 | C | 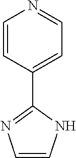 | 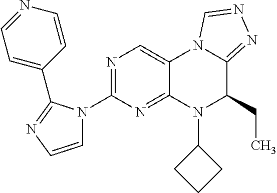 |
| 296 | D | | 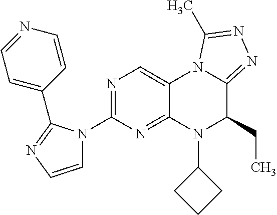 |
| 297 | M | 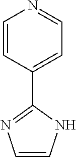 | 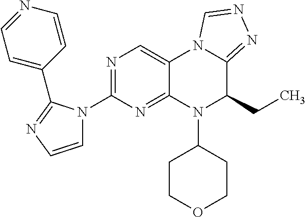 |
| 298 | M′ | | 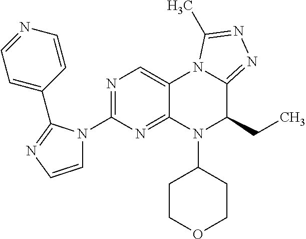 |
| 299 | M | 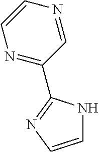 | 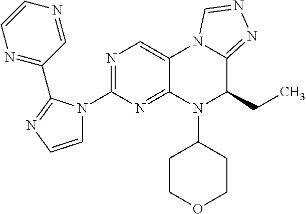 |
| 300 | M′ | | 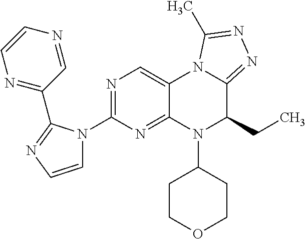 |
| 301 | O | 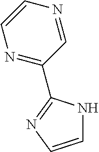 | 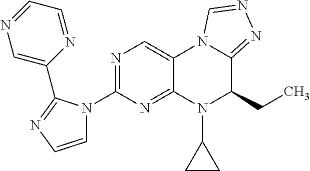 |
| 302 | O′ | | 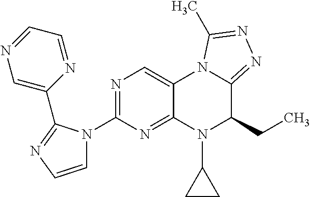 |
| 303 | O | 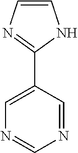 | 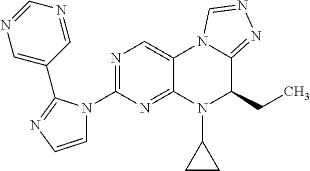 |
| 304 | O′ | | 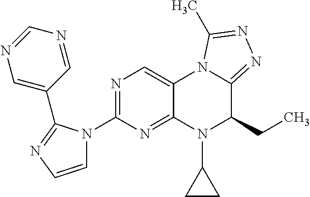 |
| 305 | O | 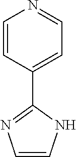 | 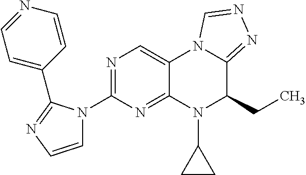 |
| 306 | O′ | | 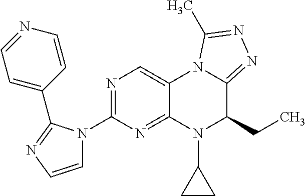 |
| 307 | G | 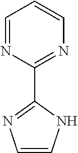 | 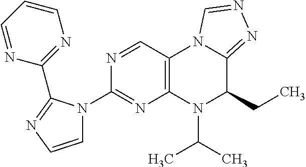 |
| 308 | H | | 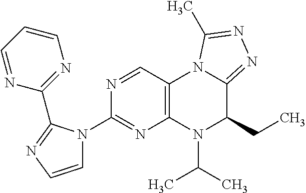 |
| 309 | G | 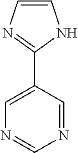 | 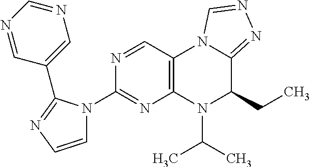 |
| 310 | H | | 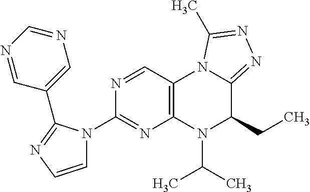 |
| 311 | G | 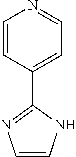 | 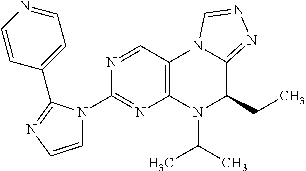 |
| 312 | H | | 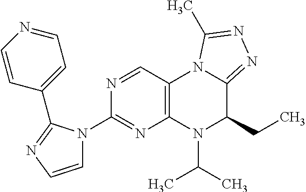 |
| 313 | G | 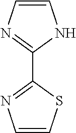 | 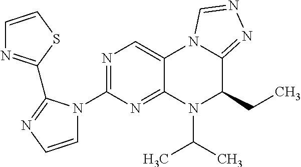 |
| 314 | H | | 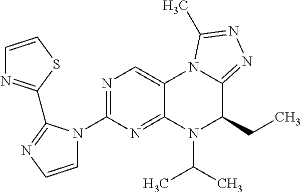 |
| 315 | G | 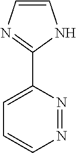 | 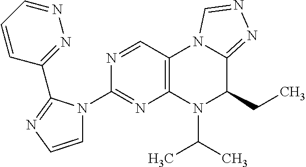 |
| 316 | H | | 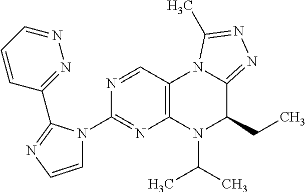 |
| 317 | G | 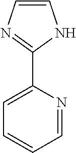 | 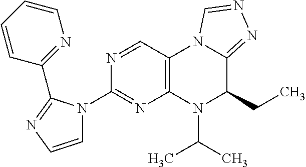 |
| 318 | H | | 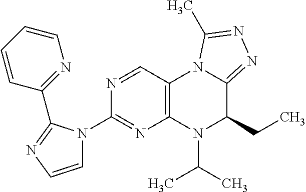 |
| 319 | G | 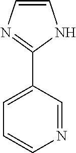 | 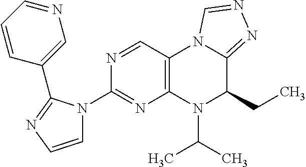 |
| 320 | H | | 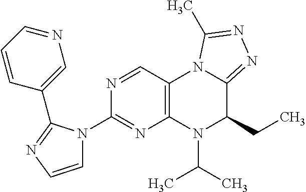 |
| 321 | Q | 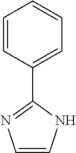 | 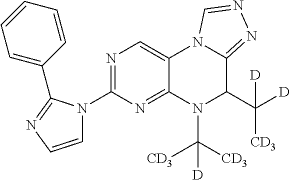 |
| 322 | Q′ | | 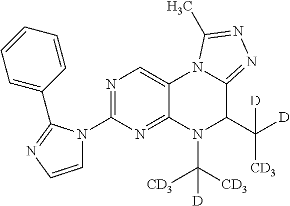 |
| 323 | C | 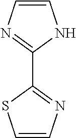 | 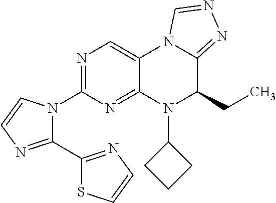 |
| 324 | D | | 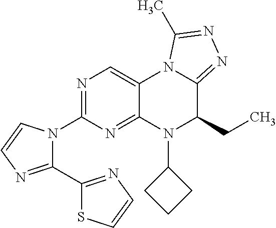 |
| 325 | M | 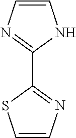 | 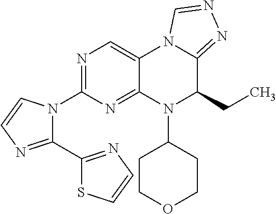 |
| 326 | M′ | | 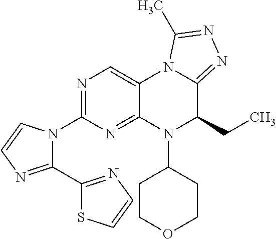 |
| 327 | Q | 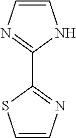 | 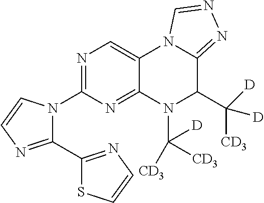 |
| 328 | Q′ | | 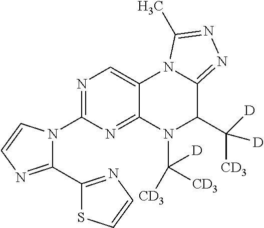 |
| 329 | E | 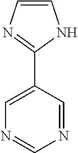 | 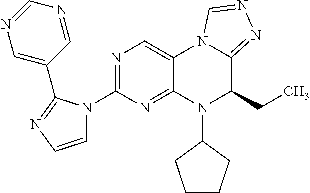 |
| 330 | F | | 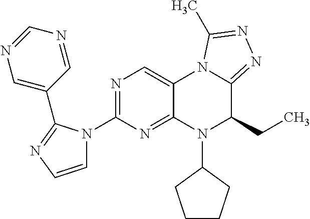 |
| 331 | G | 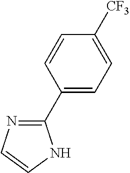 | 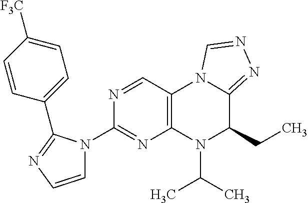 |
| 332 | H | | 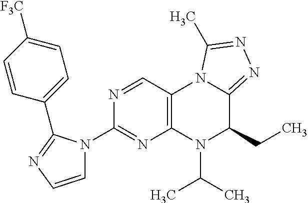 |
| 333 | C | 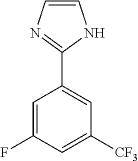 | 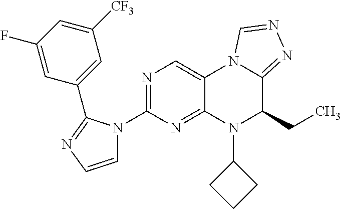 |
| 334 | D | | 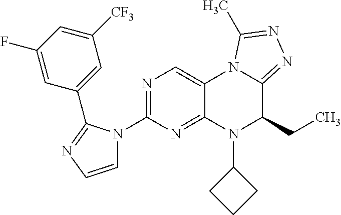 |
| 335 | C | 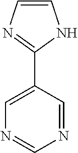 | 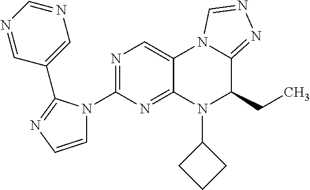 |
| 336 | D | | 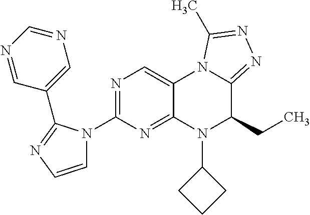 |
| 337 | V | 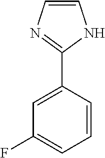 | 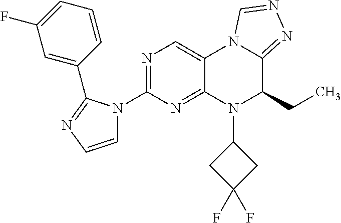 |
| 338 | V′ | | 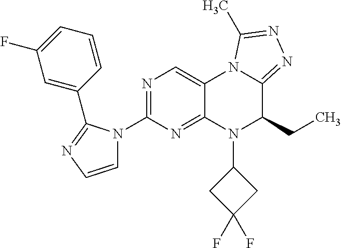 |
| 339 | G | 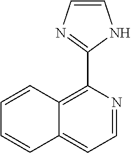 | 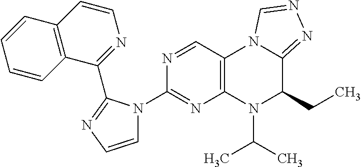 |
| 340 | H | | 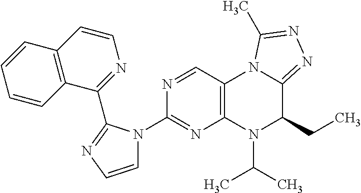 |
| 341 | G | 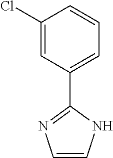 | 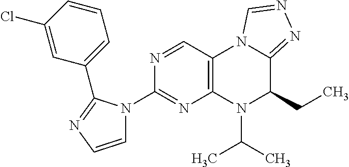 |
| 342 | H | | 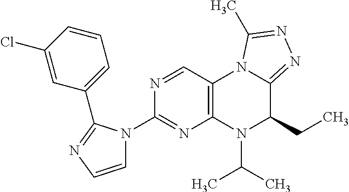 |
| 343 | G | 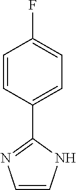 | 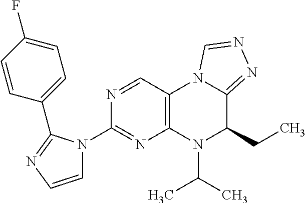 |
| 344 | H | | 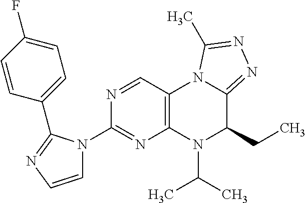 |
| 345 | C | 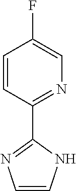 | 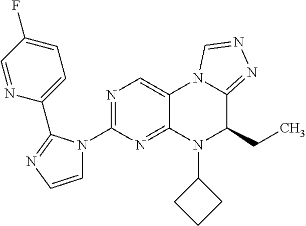 |
| 346 | D | | 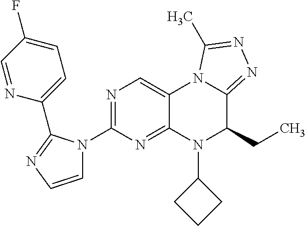 |
| 347 | W | 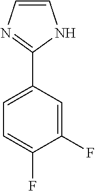 | 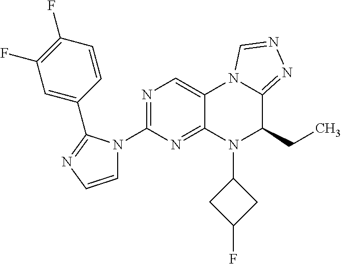 |
| 348 | W′ | | 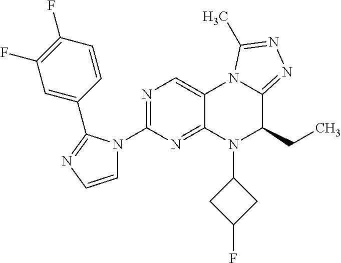 |
| 349 | E | 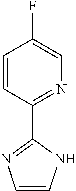 | 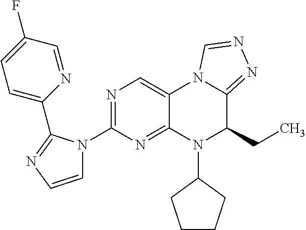 |
| 350 | F | | 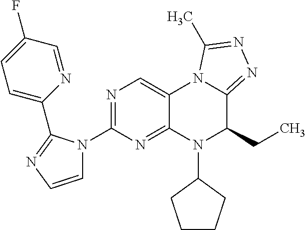 |
| 351 | ZZ | 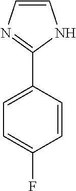 | 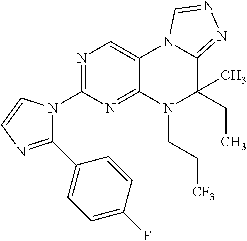 |
| 352 | ZZ′ | | 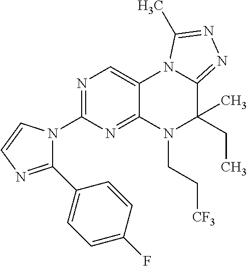 |
| 353 | E | 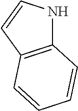 | 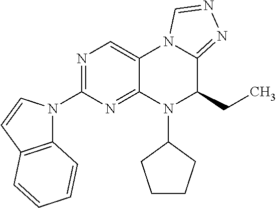 |
| 354 | F | | 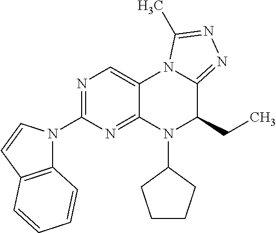 |

### Example 355

### Synthesis of ((R)-3-(1-(5-cyclopentyl-4-ethyl-4,5-dihydro-[1,2,4]triazolo[4,3-f]pteridin-7-yl)-1H-imidazol-2-yl)oxazolidin-2-one

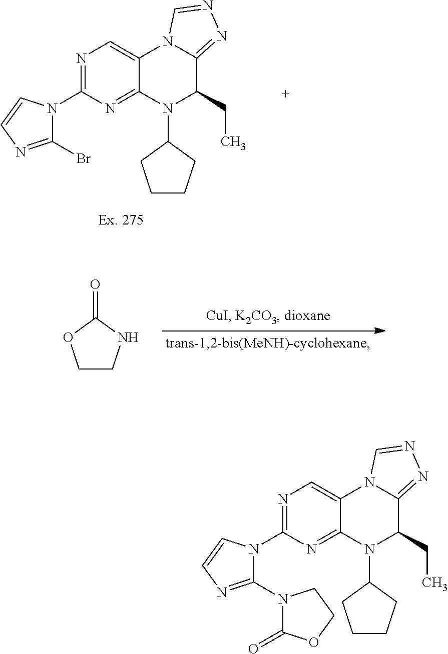

(R)-7-(2-bromo-1H-imidazol-1-yl)-5-cyclopentyl-4-ethyl-4,5-dihydro-[1,2,4]triazolo[4,3-f]pteridine (Example 275, 0.271 mmol), oxazolidin-2-one (0.406 mmol), CuI (0.054 mmol), trans-1,2-bis(methylamino)cyclohexane (0.108 mmol) and K₂CO₃ (0.542 mmol) are dissolved in 1 mL of dioxane in a screw cap vial and a stream of nitrogen is bubbled through the mixture for 2 minutes. The resulting solution is stirred at 110° C. for 18 h. The reaction mixture is filtered and concentrated, then purified by preparative HPLC to give the title compound.

Additional compounds are prepared similarly to this method, optionally replacing the compound of Example 275 with a suitable bromo compound, and/or oxazolidin-2-one with a suitable ring reactant. The following compounds are prepared:
(R)-3-(1-(5-cyclopentyl-4-ethyl-1-methyl-4,5-dihydro-[1,2,4]triazolo[4,3-f]pteridin-7-yl)-1H-imidazol-2-yl)oxazolidin-2-one (Example 356),
(R)-1-(1-(5-cyclopentyl-4-ethyl-4,5-dihydro-[1,2,4]triazolo[4,3-f]pteridin-7-yl)-1H-imidazol-2-yl)pyrrolidin-2-one (Example 357),
(R)-1-(1-(5-cyclopentyl-4-ethyl-1-methyl-4,5-dihydro-[1,2,4]triazolo[4,3-f]pteridin-7-yl)-1H-imidazol-2-yl)pyrrolidin-2-one (Example 358),
(R)-7-(2-(1H-pyrazol-1-yl)-1H-imidazol-1-yl)-5-cyclopentyl-4-ethyl-4,5-dihydro-[1,2,4]triazolo[4,3-f]pteridine (Example 359),
(R)-7-(2-(1H-pyrazol-1-yl)-1H-imidazol-1-yl)-5-cyclopentyl-4-ethyl-1-methyl-4,5-dihydro-[1,2,4]triazolo[4,3-f]pteridine (Example 360),
(R)-1-(1-(5-cyclopentyl-4-ethyl-4,5-dihydro-[1,2,4]triazolo[4,3-f]pteridin-7-yl)-1H-imidazol-2-yl)pyridin-2(1H)-one (Example 361), and
(R)-1-(1-(5-cyclopentyl-4-ethyl-1-methyl-4,5-dihydro-[1,2,4]triazolo[4,3-f]pteridin-7-yl)-1H-imidazol-2-yl)pyridin-2(1H)-one (Example 362).
   The following table provides the example number (column 1), starting compound (SC) Example number (column 2), and ring reactant (column 3), to give the compound shown in column 4.

| Ex. | | | |
|---|---|---|---|
| No. | SC | ring reactant | Compound structure |
| 356 | 276 | 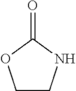 | 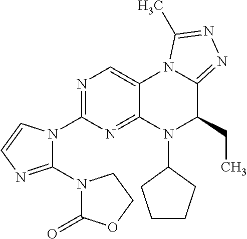 |
| 357 | 275 | 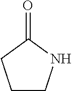 | 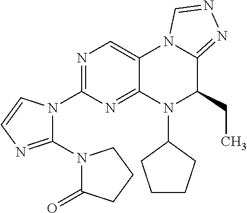 |
| 358 | 276 | | 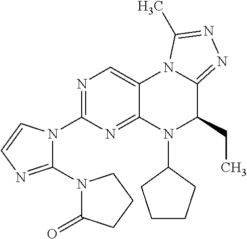 |
| 359 | 275 | 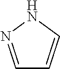 | 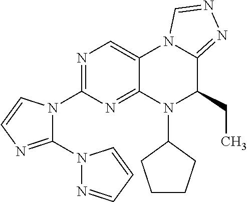 |
| 360 | 276 | | 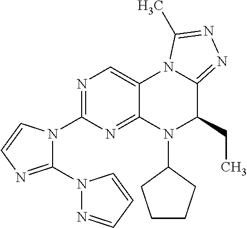 |
| 361 | 275 | 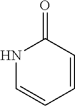 | 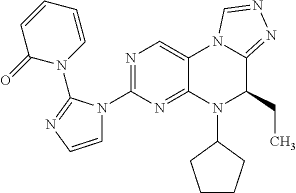 |
| 362 | 276 | | 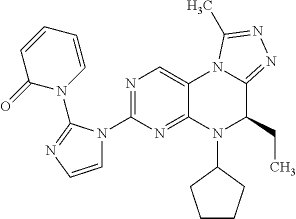 |

### Example 363

### Synthesis of (R)-5-cyclopentyl-4-ethyl-7-(2-(4-(methylsulfonyl)phenyl)-1H-imidazol-1-yl)-4,5-dihydro-[1,2,4]triazolo[4,3-f]pteridine

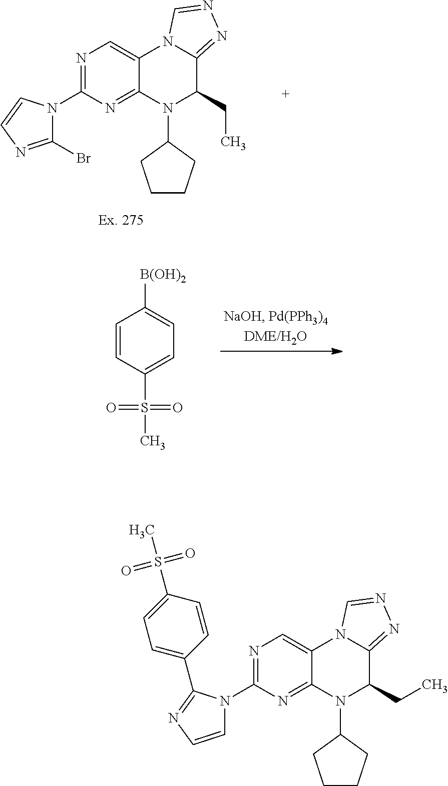

(R)-7-(2-bromo-1H-imidazol-1-yl)-5-cyclopentyl-4-ethyl-4,5-dihydro-[1,2,4]triazolo[4,3-f]pteridine (Example 275, 0.37 mmol), 4-(methylsulfonyl)phenylboronic acid (0.74 mmol), aqueous sodium hydroxide (240 μL of 3N) and Pd(PPh₃)₄ (0.037 mmol) are dissolved in 1.2 mL of DME/H₂O (5/1, v/v) and a stream of nitrogen is bubbled through the mixture for 2 minutes. The resulting solution is stirred at 90° C. for 18 h. The reaction mixture is diluted with brine, extracted with EtOAc, dried with Na₂SO₄ then purified by silica gel column chromatography and preparative HPLC to give the title compound.

Additional compounds are prepared similarly to this method, optionally replacing the compound of Example 275 with a suitable bromo compound, and/or 4-(methylsulfonyl)phenylboronic acid with a suitable boronic acid. The following compounds are prepared:
(R)-5-cyclopentyl-4-ethyl-1-methyl-7-(2-(4-(methylsulfonyl)phenyl)-1H-imidazol-1-yl)-4,5-dihydro-[1,2,4]triazolo[4,3-f]pteridine (Example 364),
(R)-7-(2-(1H-pyrazol-4-yl)-1H-imidazol-1-yl)-5-cyclopentyl-4-ethyl-4,5-dihydro-[1,2,4]triazolo[4,3-f]pteridine (Example 365),
(R)-7-(2-(1H-pyrazol-4-yl)-1H-imidazol-1-yl)-5-cyclopentyl-4-ethyl-1-methyl-4,5-dihydro-[1,2,4]triazolo[4,3-f]pteridine (Example 366),
(R)-5-cyclopentyl-4-ethyl-7-(2-(5-fluoropyridin-3-yl)-1H-imidazol-1-yl)-4,5-dihydro-[1,2,4]triazolo[4,3-f]pteridine (Example 367),
(R)-5-cyclopentyl-4-ethyl-7-(2-(5-fluoropyridin-3-yl)-1H-imidazol-1-yl)-1-methyl-4,5-dihydro-[1,2,4]triazolo[4,3-f]pteridine (Example 368),
(R)-7-(2-cyclopentenyl-1H-imidazol-1-yl)-5-cyclopentyl-4-ethyl-4,5-dihydro-[1,2,4]triazolo[4,3-f]pteridine (Example 369), and
(R)-7-(2-cyclopentenyl-1H-imidazol-1-yl)-5-cyclopentyl-4-ethyl-1-methyl-4,5-dihydro-[1,2,4]triazolo[4,3-f]pteridine (Example 370).
   The following table provides the example number (column 1), starting compound (SC) Example number (column 2), and ring reactant (column 3), to give the compound shown in column 4.

| Ex. No. | SC | Boronic acid | Compound structure |
|---|---|---|---|
| 364 | 276 | 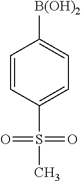 | 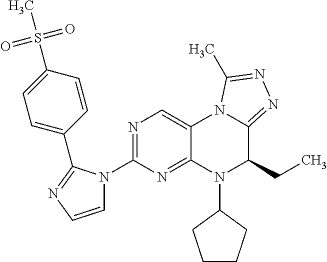 |
| 365 | 275 | 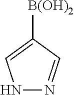 | 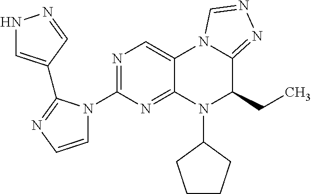 |
| 366 | 276 | | 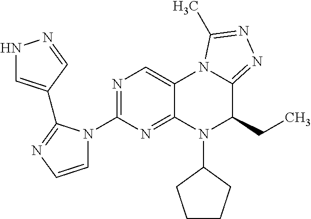 |
| 367 | 275 | 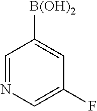 | 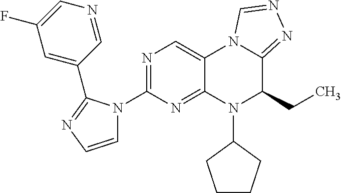 |
| 368 | 276 | | 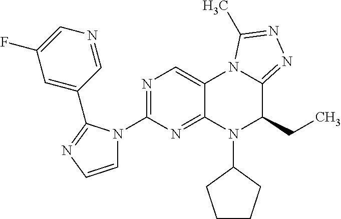 |
| 369 | 275 | 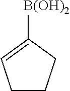 | 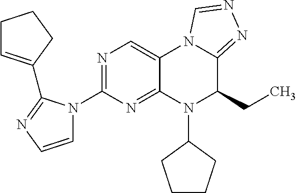 |
| 370 | 276 | | 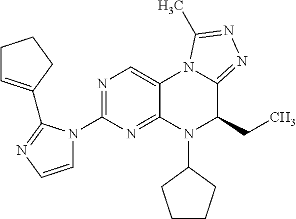 |

### Example 371-374

### Synthesis of (R)-5-cyclopentyl-7-(2-(3,6-dihydro-2H-pyran-4-yl)-1H-imidazol-1-yl)-4-ethyl-4,5-dihydro-[1,2,4]triazolo[4,3-f]pteridine (371), (R)-5-cyclopentyl-7-(2-(3,6-dihydro-2H-pyran-4-yl)-1H-imidazol-1-yl)-4-ethyl-1-methyl-4,5-dihydro-[1,2,4] triazolo[4,3-f]pteridine (372), (R)-5-cyclopentyl-4-ethyl-7-(2-(tetrahydro-2H-pyran-4-yl)-1H-imidazol-1-yl)-4,5-dihydro-[1,2,4]triazolo[4,3-f]pteridine (373), and (R)-5-cyclopentyl-4-ethyl-1-methyl-7-(2-(tetrahydro-2H-pyran-4-yl)-1H-imidazol-1-yl)-4,5-dihydro-[1,2,4]triazolo[4,3-f]pteridine (374)

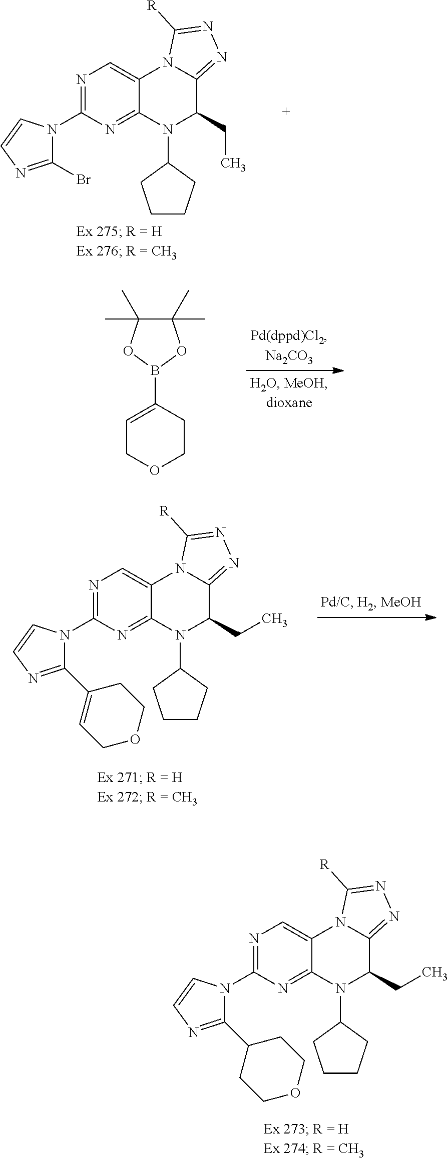

(R)-7-(2-bromo-1H-imidazol-1-yl)-5-cyclopentyl-4-ethyl-4,5-dihydro-[1,2,4]triazolo[4,3-f]pteridine or (R)-7-(2-bromo-1H-imidazol-1-yl)-5-cyclopentyl-4-ethyl-1-methyl-4,5-dihydro-[1,2,4]triazolo[4,3-f]pteridine (Example 275 or 276, 1 eq) in dioxane/water/MeOH (2 mL/0.5 mL/0.05 mL) is combined with Pd(dppf)Cl₂ (0.2 eq), Na₂CO₃ (3 eq), and 2-(3,6-dihydro-2H-pyran-4-yl)-4,4,5,5-tetramethyl-1,3,2-dioxaborolane (2 eq). The reaction mixture is stirred at 110° C. overnight. This is diluted with EtOAc and a saturated NaHCO₃ solution. The layers are separated and the aqueous layer is extracted with EtOAc (2×25 mL). The organic layers are dried over MgSO₄, filtered, and concentrated under reduced pressure. The crude material is purified by MPLC and further purified by preparative HPLC to give Example 371 or 372.

To Example 371 or 372 in 5 mL of MeOH, Pd/C (20 mg) is added. This reaction mixture is placed under a hydrogen balloon until all the starting material is consumed. The resulting mixture is filtered through a plug of Celite, and the plug is washed several times with EtOAc. The mixture is concentrated under reduced pressure and further purified by preparative HPLC to give Examples 373 and 374.

### Example 375

### Synthesis of (R)-5-cyclopentyl-4-ethyl-7-(2-(pyrrolidin-1-yl)-1H-imidazol-1-yl)-4,5-dihydro-[1,2,4]triazolo[4,3-f]pteridine

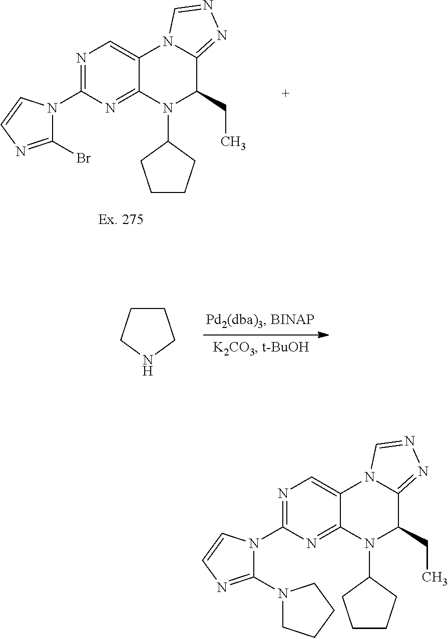

(R)-7-(2-bromo-1H-imidazol-1-yl)-5-cyclopentyl-4-ethyl-4,5-dihydro-[1,2,4]triazolo[4,3-f]pteridine (Example 275, 0.37 mmol), pyrrolidine (0.74 mmol), Pd₂ dba₃.CHCl₃ (0.074 mmol), BINAP (0.11 mmol) and K₂CO₃ (1.11 mmol) are dissolved in 1 mL of degassed t-BuOH and the resulting solution is heated at 130° C. for 18 h. The reaction mixture is diluted with EtOAc and washed with brine. The organic extracts are dried with Na₂SO₄, filtered and evaporated, and the residue is purified by preparative HPLC to give the title compound.

Additional compounds are prepared similarly to this method, optionally replacing the compound of Example 275 with a suitable bromo compound, and/or pyrrolidine with a suitable ring reactant. The following compounds are prepared:
(R)-5-cyclopentyl-4-ethyl-1-methyl-7-(2-(pyrrolidin-1-yl)-1H-imidazol-1-yl)-4,5-dihydro-[1,2,4]triazolo[4,3-f]pteridine (Example 376),
(R)-4-(1-(5-cyclopentyl-4-ethyl-4,5-dihydro-[1,2,4]triazolo[4,3-f]pteridin-7-yl)-1H-imidazol-2-yl)morpholine (Example 377),
(R)-4-(1-(5-cyclopentyl-4-ethyl-1-methyl-4,5-dihydro-[1,2,4]triazolo[4,3-f]pteridin-7-yl)-1H-imidazol-2-yl)morpholine (Example 378),
(R)-5-cyclopentyl-4-ethyl-7-(2-(4-methylpiperazin-1-yl)-1H-imidazol-1-yl)-4,5-dihydro-[1,2,4]triazolo[4,3-f]pteridine (Example 379), and
(R)-5-cyclopentyl-4-ethyl-1-methyl-7-(2-(4-methylpiperazin-1-yl)-1H-imidazol-1-yl)-4,5-dihydro-[1,2,4]triazolo[4,3-f]pteridine (Example 380).
   The following table provides the example number (column 1), starting compound (SC) Example number (column 2), and ring reactant (column 3), to give the compound shown in column 4.

| Ex. | | | |
|---|---|---|---|
| No. | SC | ring reactant | Compound structure |
| 376 | 276 | 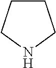 | 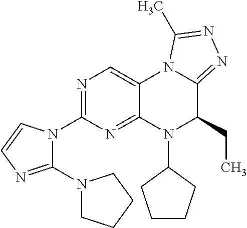 |
| 377 | 275 | 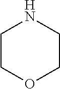 | 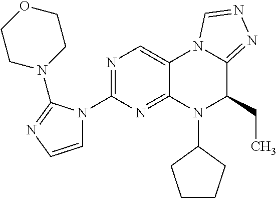 |
| 378 | 276 | | 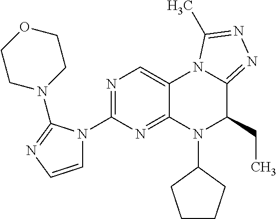 |
| 379 | 275 | 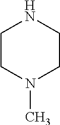 | 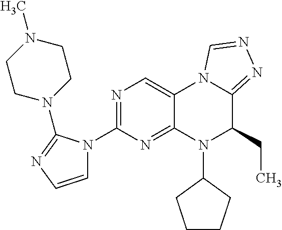 |
| 380 | 276 | | 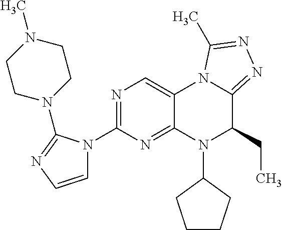 |

### Example 381 and Example 382

### Synthesis of (R)-tert-butyl 4-(5-cyclopentyl-4-ethyl-4,5-dihydro-[1,2,4]triazolo[4,3-f]pteridin-7-yl)pyridin-3-ylcarbamate (381) and (R)-4-(5-cyclopentyl-4-ethyl-4,5-dihydro-[1,2,4]triazolo[4,3-f]pteridin-7-yl)pyridin-3-amine (382)

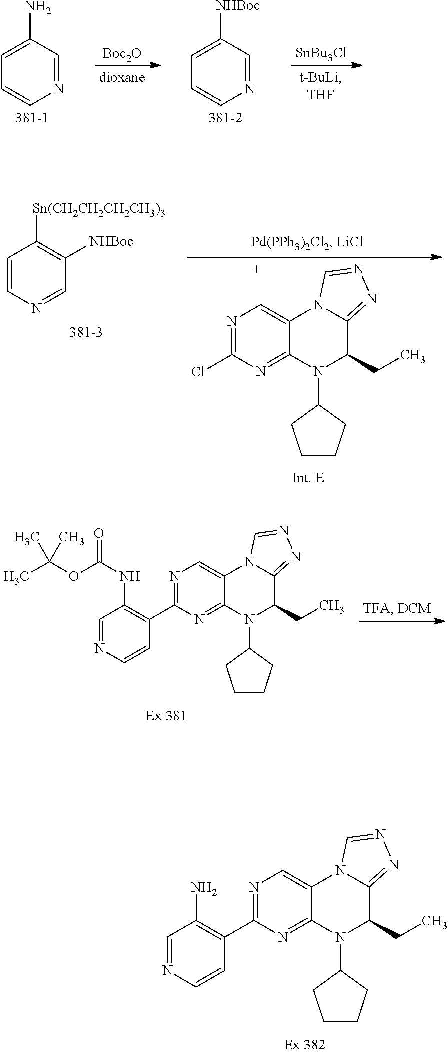

To a solution of pyridine-3-amine (compound 381-1, 9.4 g, 1 eq) in 300 mL of dioxane, Boc₂O (21.8 g, 1 eq) was added and the mixture was stirred at 60° C. for 18 h. The mixture was cooled to rt and the solvent was removed under reduced pressure. water was added to the residue and it was extracted with EtOAc. The organic layer was dried with Na₂SO₄, then concentrated and the residue was purified by a silica gel column chromatography to give the desired tert-butyl pyridin-3-ylcarbamate (compound 381-2).

To a solution of tert-butyl pyridin-3-ylcarbamate (compound 381-2, 1 eq) in dry THF, tert-butyl lithium (3 eq, in hexanes) was added dropwise. The mixture was stirred for 2 h under Ar at −78° C. and 2 h at −20° C., then SnBu₃C1 (3 eq) was added dropwise at −78° C. The mixture was stirred for 1 h at −78° C. under Ar, then the mixture was warmed to rt and stirred for 18 h under Ar. Water was added and extracted with EtOAc, the organic layer was dried with Na₂SO₄, concentrated and the residue was purified by silica column chromatography to give the desired tert-butyl 4-(tributylstannyl)pyridin-3-ylcarbamate (compound 381-3).

Intermediate E (1 eq), compound 381-3 (2 eq), Pd(PPh₃)₂Cl₂ (0.1 eq), and LiC1 (5 eq) are suspended in toluene and protected with Ar. The resulting mixture is stirred at 110° C. for 52 h. The mixture is cooled to rt and water is added and extracted with EtOAc. The organic layer is dried with Na₂SO₄, then concentrated and the residue is purified by silica column to give Example 381.

Three mL of TFA is added to a solution of Example 381 in 3 mL of DCM. The mixture is stirred for 4 h at rt, and the solvent is removed under reduced pressure. Aqueous Na₂CO₃ is added and extracted with EtOAc. The organic layer is dried with Na₂SO₄, concentrated and the residue is purified by silica column to give Example 382.

Additional compounds are prepared similarly to this method, optionally replacing pyridine-3-amine with a suitable amine compound and/or replacing Intermediate E with a suitable Intermediate, where the Boc protected compound may be isolated or deprotected to give the amine. The following compounds are prepared:
(R)-tert-butyl 4-(5-cyclopentyl-4-ethyl-1-methyl-4,5-dihydro-[1,2,4]triazolo[4,3-f]pteridin-7-yl)pyridin-3-ylcarbamate (Example 383),
(R)-4-(5-cyclopentyl-4-ethyl-1-methyl-4,5-dihydro-[1,2,4]triazolo[4,3-f]pteridin-7-yl)pyridin-3-amine (Example 384),
(R)-tert-butyl 4-(4-ethyl-5-isopropyl-4,5-dihydro-[1,2,4]triazolo[4,3-f]pteridin-7-yl)pyridin-3-ylcarbamate (Example 385),
(R)-4-(4-ethyl-5-isopropyl-4,5-dihydro-[1,2,4]triazolo[4,3-f]pteridin-7-yl)pyridin-3-amine (Example 386),
(R)-tert-butyl 4-(4-ethyl-5-isopropyl-1-methyl-4,5-dihydro-[1,2,4]triazolo[4,3-f]pteridin-7-yl)pyridin-3-ylcarbamate (Example 387),
(R)-4-(4-ethyl-5-isopropyl-1-methyl-4,5-dihydro-[1,2,4]triazolo[4,3-f]pteridin-7-yl)pyridin-3-amine (Example 388),
(R)-tert-butyl (4-(5-cyclopentyl-4-ethyl-4,5-dihydro-[1,2,4]triazolo[4,3-f]pteridin-7-yl)pyridin-3-yl)methylcarbamate (Example 389),
(R)-(4-(5-cyclopentyl-4-ethyl-4,5-dihydro-[1,2,4]triazolo[4,3-f]pteridin-7-yl)pyridin-3-yl)methanamine (Example 390),
(R)-tert-butyl (4-(5-cyclopentyl-4-ethyl-1-methyl-4,5-dihydro-[1,2,4]triazolo[4,3-f]pteridin-7-yl)pyridin-3-yl)methylcarbamate (Example 391),
(R)-(4-(5-cyclopentyl-4-ethyl-1-methyl-4,5-dihydro-[1,2,4]triazolo[4,3-f]pteridin-7-yl)pyridin-3-yl)methanamine (Example 392),
(R)-tert-butyl (4-(4-ethyl-5-isopropyl-4,5-dihydro-[1,2,4]triazolo[4,3-f]pteridin-7-yl)pyridin-3-yl)methylcarbamate (Example 393),
(R)-(4-(4-ethyl-5-isopropyl-4,5-dihydro-[1,2,4]triazolo[4,3-f]pteridin-7-yl)pyridin-3-yl)methanamine (Example 394),
(R)-tert-butyl (4-(4-ethyl-5-isopropyl-1-methyl-4,5-dihydro-[1,2,4]triazolo[4,3-f]pteridin-7-yl)pyridin-3-yl)methylcarbamate (Example 395),
(R)-(4-(4-ethyl-5-isopropyl-1-methyl-4,5-dihydro-[1,2,4]triazolo[4,3-f]pteridin-7-yl)pyridin-3-yl)methanamine (Example 396),
   The following table provides the example number (column 1), starting amine compound (column 2), and Intermediate (column 3), to give the compound shown in column 4.

| Ex. | Amine | | |
|---|---|---|---|
| No. | compound | Int. | Compound structure |
| 383 | 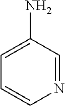 | F | 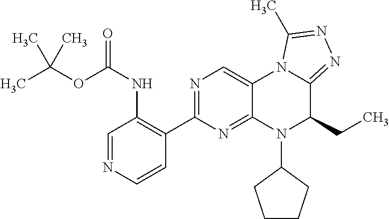 |
| 384 | 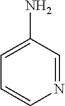 | | 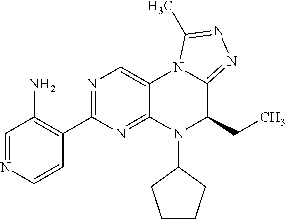 |
| 385 | 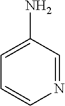 | G | 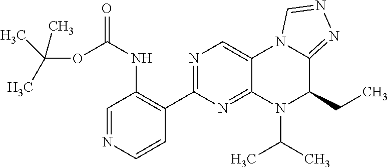 |
| 386 | | | 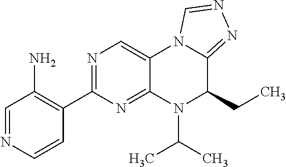 |
| 387 | 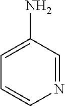 | H | 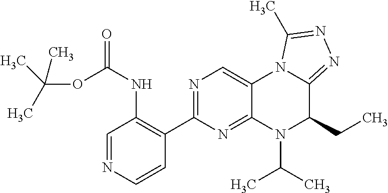 |
| 388 | | | 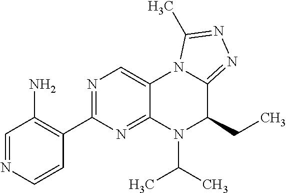 |
| 389 | 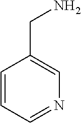 | E | 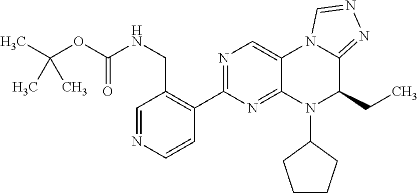 |
| 390 | | | 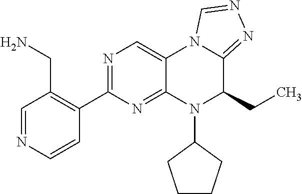 |
| 391 | 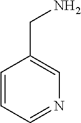 | F | 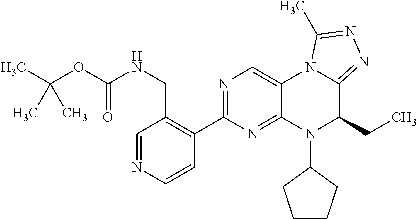 |
| 392 | | | 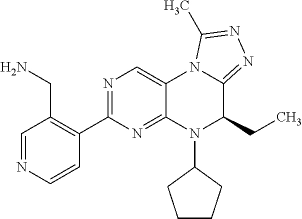 |
| 393 | 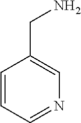 | G | 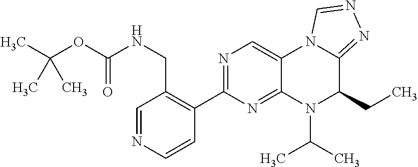 |
| 394 | | | 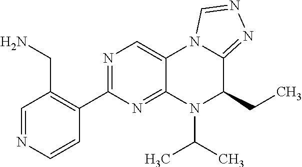 |
| 395 | 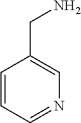 | H | 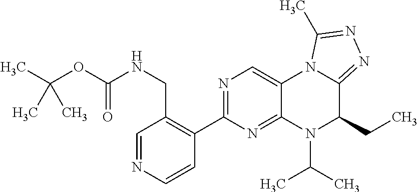 |
| 396 | | | 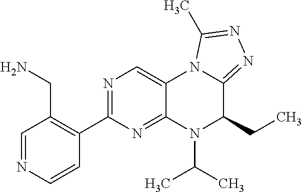 |

### Example 397

### Synthesis of (R)-N-(4-(5-cyclopentyl-4-ethyl-4,5-dihydro-[1,2,4]triazolo[4,3-f]pteridin-7-yl)pyridin-3-yl)benzamide

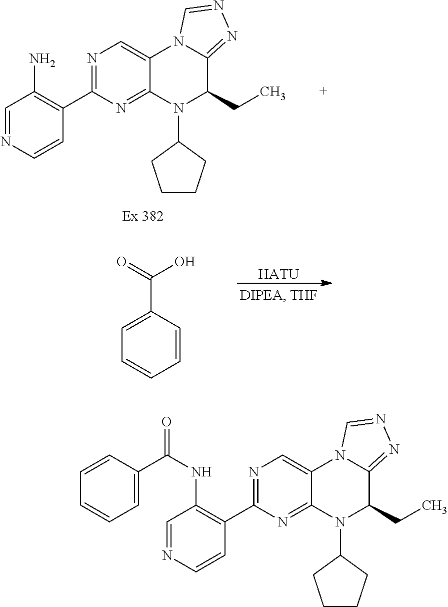

A mixture of (R)-4-(5-cyclopentyl-4-ethyl-4,5-dihydro-[1,2,4]triazolo[4,3-f]pteridin-7-yl)pyridin-3-amine (Example 382, 1 eq), benzoic acid (3 eq), HATU (3 eq), and DIPEA (4 eq) in dry THF under Ar is stirred at 90° C. for 18 h. The mixture is cooled to rt and water is added and extracted with EtOAc. The organic layer is dried with Na₂SO₄, concentrated and the residue is purified by silica gel column to give the title compound.

Additional compounds are prepared similarly to this method, optionally replacing the compound of Example 382 with a suitable amine compound and/or replacing benzoic acid with a suitable carboxylic acid. The following compounds are prepared:
(R)-N-(4-(5-cyclopentyl-4-ethyl-1-methyl-4,5-dihydro-[1,2,4]triazolo[4,3-f]pteridin-7-yl)pyridin-3-yl)benzamide (Example 398),
(R)-N-(4-(4-ethyl-5-isopropyl-4,5-dihydro-[1,2,4]triazolo[4,3-f]pteridin-7-yl)pyridin-3-yl)-3,3-dimethylbutanamide (Example 399),
(R)-N-(4-(4-ethyl-5-isopropyl-1-methyl-4,5-dihydro-[1,2,4]triazolo[4,3-f]pteridin-7-yl)pyridin-3-yl)-3,3-dimethylbutanamide (Example 400),
(R)-N-(4-(4-ethyl-5-isopropyl-4,5-dihydro-[1,2,4]triazolo[4,3-f]pteridin-7-yl)pyridin-3-yl)benzamide (Example 401),
(R)-N-(4-(4-ethyl-5-isopropyl-1-methyl-4,5-dihydro-[1,2,4]triazolo[4,3-f]pteridin-7-yl)pyridin-3-yl)benzamide (Example 402),
(R)-N-(4-(5-cyclopentyl-4-ethyl-4,5-dihydro-[1,2,4]triazolo[4,3-f]pteridin-7-yl)pyridin-3-yl)-3,3-dimethylbutanamide (Example 403),
(R)-N-(4-(5-cyclopentyl-4-ethyl-1-methyl-4,5-dihydro-[1,2,4]triazolo[4,3-f]pteridin-7-yl)pyridin-3-yl)-3,3-dimethylbutanamide (Example 404),
(R)-N-(4-(5-cyclopentyl-4-ethyl-4,5-dihydro-[1,2,4]triazolo[4,3-f]pteridin-7-yl)pyridin-3-yl)acetamide (Example 405),
(R)-N-(4-(5-cyclopentyl-4-ethyl-1-methyl-4,5-dihydro-[1,2,4]triazolo[4,3-f]pteridin-7-yl)pyridin-3-yl)acetamide (Example 406),
(R)-N-((4-(5-cyclopentyl-4-ethyl-4,5-dihydro-[1,2,4]triazolo[4,3-f]pteridin-7-yl)pyridin-3-yl)methyl)acetamide (Example 407),
(R)-N-((4-(5-cyclopentyl-4-ethyl-1-methyl-4,5-dihydro-[1,2,4]triazolo[4,3-f]pteridin-7-yl)pyridin-3-yl)methyl)acetamide (Example 408),
(R)-N-((4-(5-cyclopentyl-4-ethyl-4,5-dihydro-[1,2,4]triazolo[4,3-f]pteridin-7-yl)pyridin-3-yl)methyl)benzamide (Example 409),
(R)-N-((4-(5-cyclopentyl-4-ethyl-1-methyl-4,5-dihydro-[1,2,4]triazolo[4,3-f]pteridin-7-yl)pyridin-3-yl)methyl)benzamide (Example 410),
(R)-N-((4-(4-ethyl-5-isopropyl-4,5-dihydro-[1,2,4]triazolo[4,3-f]pteridin-7-yl)pyridin-3-yl)methyl)acetamide (Example 411),
(R)-N-((4-(4-ethyl-5-isopropyl-1-methyl-4,5-dihydro-[1,2,4]triazolo[4,3-f]pteridin-7-yl)pyridin-3-yl)methyl)acetamide (Example 412),
(R)-N-((4-(4-ethyl-5-isopropyl-4,5-dihydro-[1,2,4]triazolo[4,3-f]pteridin-7-yl)pyridin-3-yl)methyl)benzamide (Example 413), and
(R)-N-((4-(4-ethyl-5-isopropyl-1-methyl-4,5-dihydro-[1,2,4]triazolo[4,3-f]pteridin-7-yl)pyridin-3-yl)methyl)benzamide (Example 414).
   The following table provides the example number (column 1), starting compound (SC) Example number (column 2), and carboxylic acid (column 3), to give the compound shown in column 4.

| Ex. | | | |
|---|---|---|---|
| No. | SC | Carboxylic acid | Compound structure |
| 398 | 384 | 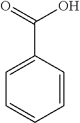 | 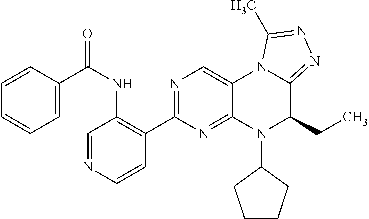 |
| 399 | 386 | 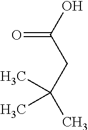 | 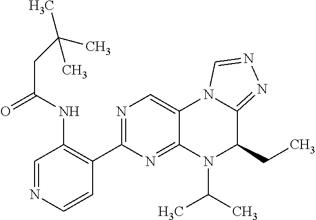 |
| 400 | 388 | | 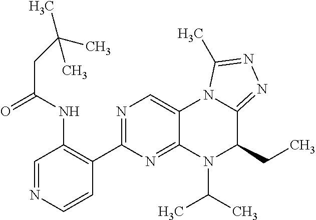 |
| 401 | 386 | 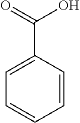 | 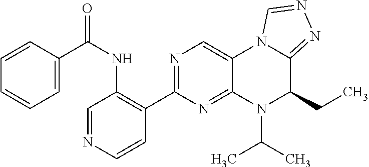 |
| 402 | 388 | | 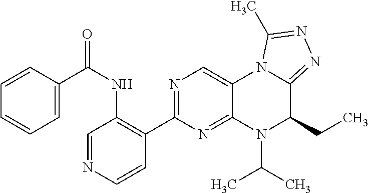 |
| 403 | 382 | 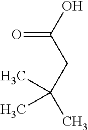 | 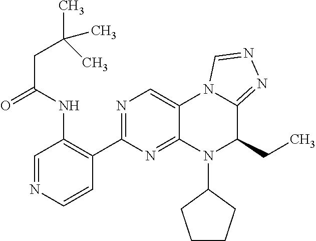 |
| 404 | 384 | | 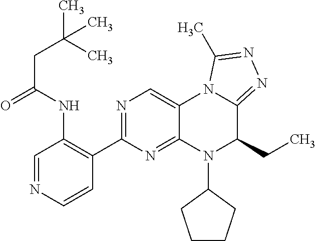 |
| 405 | 382 | 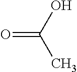 | 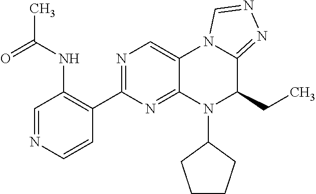 |
| 406 | 384 | | 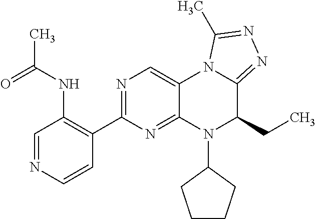 |
| 407 | 390 | 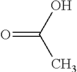 | 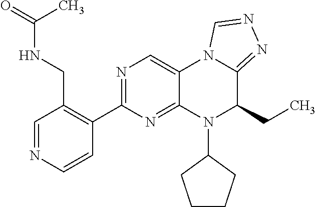 |
| 408 | 392 | | 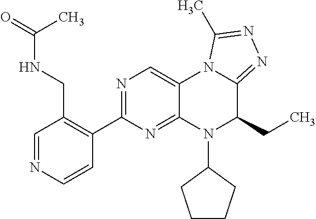 |
| 409 | 390 | 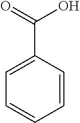 | 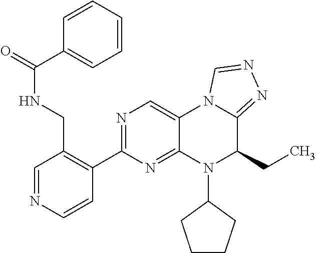 |
| 410 | 392 | | 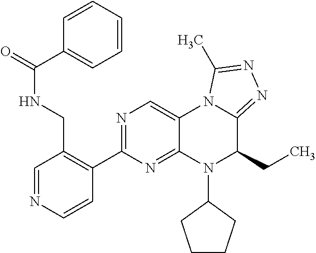 |
| 411 | 394 | 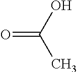 | 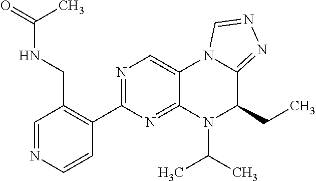 |
| 412 | 396 | | 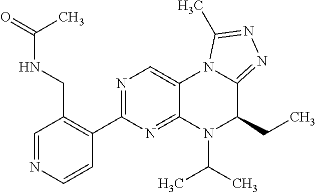 |
| 413 | 394 | 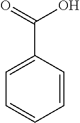 | 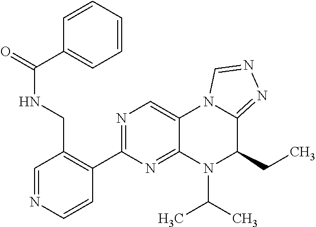 |
| 414 | 396 | | 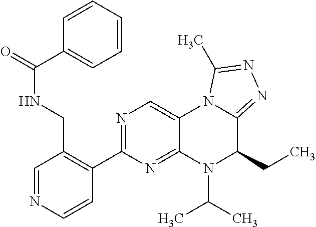 |

### Example 415 and Example 416

### Synthesis of (R)-1-(1-(5-cyclobutyl-4-ethyl-4,5-dihydro-[1,2,4]triazolo[4,3-f]pteridin-7-yl)-1H-imidazol-2-yl)pyrrolidine-2,5-dione (415) and (R)-1-(1-(5-cyclobutyl-4-ethyl-1-methyl-4,5-dihydro-[1,2,4]triazolo[4,3-f]pteridin-7-yl)-1H-imidazol-2-yl)pyrrolidine-2,5-dione (416)

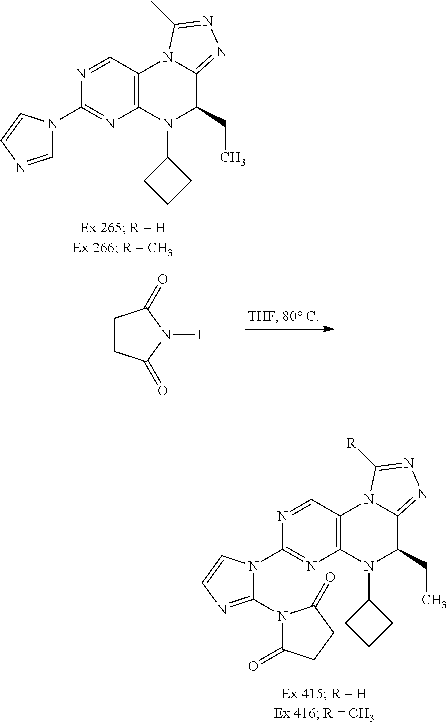

(R)-5-cyclobutyl-4-ethyl-7-(1H-imidazol-1-yl)-4,5-dihydro-[1,2,4]triazolo[4,3-f]pteridine or (R)-5-cyclobutyl-4-ethyl-7-(1H-imidazol-1-yl)-1-methyl-4,5-dihydro-[1,2,4]triazolo[4,3-f]pteridine (Example 265 or 266, 0.448 mmol) is dissolved in 2 mL of THF and NIS (0.896 mmol) is added. The solution is stirred at 80° C. for 6 hours after which the solution is concentrated and purified by preparative HPLC to give the title compounds.

### Example 417

### Synthesis of (R)-methyl 4-(5-cyclopentyl-4-ethyl-4,5-dihydro-[1,2,4]triazolo[4,3-f]pteridin-7-yl)pyridin-3-ylcarbamate

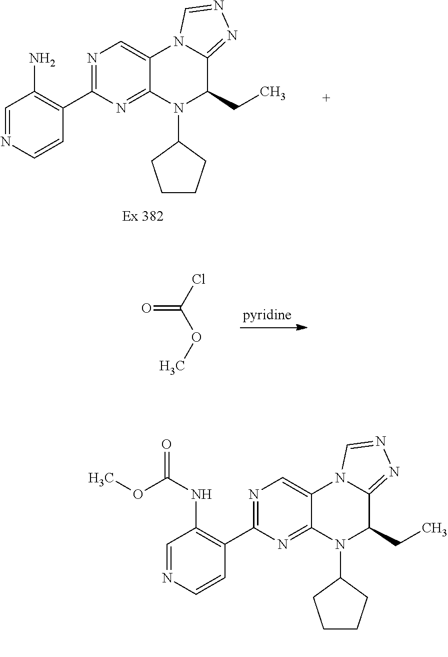

A mixture of (R)-4-(5-cyclopentyl-4-ethyl-4,5-dihydro-[1,2,4]triazolo[4,3-f]pteridin-7-yl)pyridin-3-amine (Example 382, 1 eq) and chloromethylcarbonate (10 eq) in dry pyridine under Ar is stirred at 80° C. overnight. The mixture is cooled to rt and water is added, then extracted with EtOAc. The organic layer is dried with Na₂SO₄, then concentrated and the residue is purified by a silica gel column to give the title compound.

Additional compounds are prepared similarly to this method, optionally replacing the compound of Example 382 with a suitable amine compound and/or replacing chloromethylcarbonate with acetyl chloride or a suitable sulfonyl chloride. The following compounds are prepared:
(R)-methyl 4-(5-cyclopentyl-4-ethyl-1-methyl-4,5-dihydro-[1,2,4]triazolo[4,3-f]pteridin-7-yl)pyridin-3-ylcarbamate (Example 418),
(R)-N-(4-(5-cyclopentyl-4-ethyl-4,5-dihydro-[1,2,4]triazolo[4,3-f]pteridin-7-yl)pyridin-3-yl)methanesulfonamide (Example 419),
(R)-N-(4-(5-cyclopentyl-4-ethyl-1-methyl-4,5-dihydro-[1,2,4]triazolo[4,3-f]pteridin-7-yl)pyridin-3-yl)methanesulfonamide (Example 420),
(R)-methyl 4-(4-ethyl-5-isopropyl-4,5-dihydro-[1,2,4]triazolo[4,3-f]pteridin-7-yl)pyridin-3-ylcarbamate (Example 421),
(R)-methyl 4-(4-ethyl-5-isopropyl-1-methyl-4,5-dihydro-[1,2,4]triazolo[4,3-f]pteridin-7-yl)pyridin-3-ylcarbamate (Example 422),
(R)-N-(4-(4-ethyl-5-isopropyl-4,5-dihydro-[1,2,4]triazolo[4,3-f]pteridin-7-yl)pyridin-3-yl)acetamide (Example 423),
(R)-N-(4-(4-ethyl-5-isopropyl-1-methyl-4,5-dihydro-[1,2,4]triazolo[4,3-f]pteridin-7-yl)pyridin-3-yl)acetamide (Example 424),
(R)-N-(4-(4-ethyl-5-isopropyl-4,5-dihydro-[1,2,4]triazolo[4,3-f]pteridin-7-yl)pyridin-3-yl)methanesulfonamide (Example 425),
(R)-N-(4-(4-ethyl-5-isopropyl-1-methyl-4,5-dihydro-[1,2,4]triazolo[4,3-f]pteridin-7-yl)pyridin-3-yl)methanesulfonamide (Example 426),
(R)-N-(4-(4-ethyl-5-isopropyl-4,5-dihydro-[1,2,4]triazolo[4,3-f]pteridin-7-yl)pyridin-3-yl)benzenesulfonamide (Example 427),
(R)-N-(4-(4-ethyl-5-isopropyl-1-methyl-4,5-dihydro-[1,2,4]triazolo[4,3-f]pteridin-7-yl)pyridin-3-yl)benzenesulfonamide (Example 428),
(R)-methyl (4-(5-cyclopentyl-4-ethyl-4,5-dihydro-[1,2,4]triazolo[4,3-f]pteridin-7-yl)pyridin-3-yl)methylcarbamate (Example 429),
(R)-methyl (4-(5-cyclopentyl-4-ethyl-1-methyl-4,5-dihydro-[1,2,4]triazolo[4,3-f]pteridin-7-yl)pyridin-3-yl)methylcarbamate (Example 430),
(R)-N-((4-(5-cyclopentyl-4-ethyl-4,5-dihydro-[1,2,4]triazolo[4,3-f]pteridin-7-yl)pyridin-3-yl)methyl)methanesulfonamide (Example 431),
(R)-N-((4-(5-cyclopentyl-4-ethyl-1-methyl-4,5-dihydro-[1,2,4]triazolo[4,3-f]pteridin-7-yl)pyridin-3-yl)methyl)methanesulfonamide (Example 432),
(R)-N-((4-(5-cyclopentyl-4-ethyl-4,5-dihydro-[1,2,4]triazolo[4,3-f]pteridin-7-yl)pyridin-3-yl)methyl)benzenesulfonamide (Example 433),
(R)-N-((4-(5-cyclopentyl-4-ethyl-1-methyl-4,5-dihydro-[1,2,4]triazolo[4,3-f]pteridin-7-yl)pyridin-3-yl)methyl)benzenesulfonamide (Example 434),
(R)-N-(4-(5-cyclopentyl-4-ethyl-4,5-dihydro-[1,2,4]triazolo[4,3-f]pteridin-7-yl)pyridin-3-yl)benzenesulfonamide (Example 435),
(R)-N-(4-(5-cyclopentyl-4-ethyl-1-methyl-4,5-dihydro-[1,2,4]triazolo[4,3-f]pteridin-7-yl)pyridin-3-yl)benzenesulfonamide (Example 436),
(R)-methyl (4-(4-ethyl-5-isopropyl-4,5-dihydro-[1,2,4]triazolo[4,3-f]pteridin-7-yl)pyridin-3-yl)methylcarbamate (Example 437),
(R)-methyl (4-(4-ethyl-5-isopropyl-1-methyl-4,5-dihydro-[1,2,4]triazolo[4,3-f]pteridin-7-yl)pyridin-3-yl)methylcarbamate (Example 438),
(R)-N-((4-(4-ethyl-5-isopropyl-4,5-dihydro-[1,2,4]triazolo[4,3-f]pteridin-7-yl)pyridin-3-yl)methyl)methanesulfonamide (Example 439),
(R)-N-((4-(4-ethyl-5-isopropyl-1-methyl-4,5-dihydro-[1,2,4]triazolo[4,3-f]pteridin-7-yl)pyridin-3-yl)methyl)methanesulfonamide (Example 440),
(R)-N-((4-(4-ethyl-5-isopropyl-4,5-dihydro-[1,2,4]triazolo[4,3-f]pteridin-7-yl)pyridin-3-yl)methyl)benzenesulfonamide (Example 441), and
(R)-N-((4-(4-ethyl-5-isopropyl-1-methyl-4,5-dihydro-[1,2,4]triazolo[4,3-f]pteridin-7-yl)pyridin-3-yl)methyl)benzenesulfonamide (Example 442).
   The following table provides the example number (column 1), starting compound (SC) Example number (column 2), and chloride reactant (column 3), to give the compound shown in column 4.

| Ex. | | | |
|---|---|---|---|
| No. | SC | Chloride reactant | Compound structure |
| 418 | 384 | 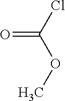 | 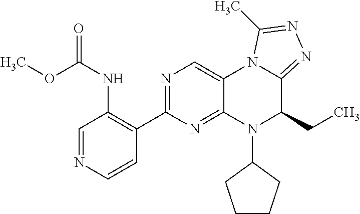 |
| 419 | 382 | 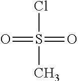 | 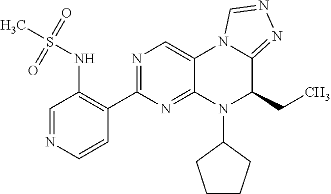 |
| 420 | 384 | | 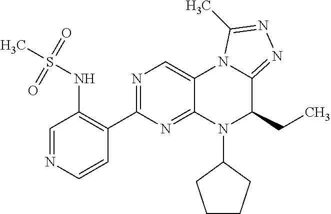 |
| 421 | 386 | 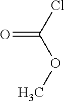 | 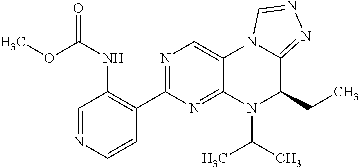 |
| 422 | 388 | | 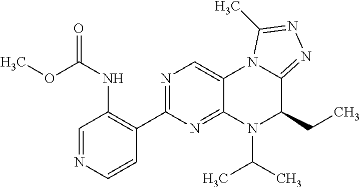 |
| 423 | 386 | 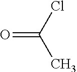 | 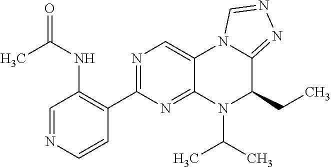 |
| 424 | 388 | | 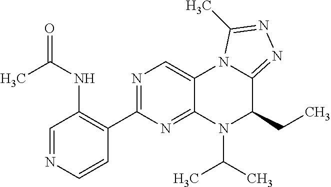 |
| 425 | 386 | 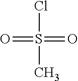 | 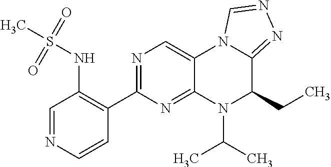 |
| 426 | 388 | | 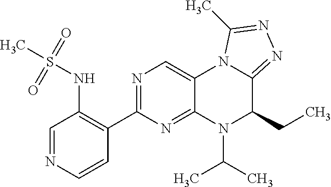 |
| 427 | 386 | 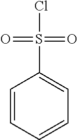 | 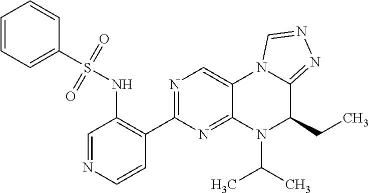 |
| 428 | 388 | | 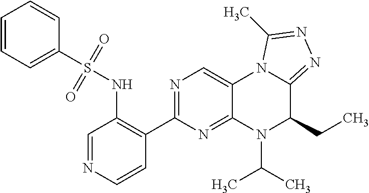 |
| 429 | 390 | 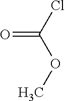 | 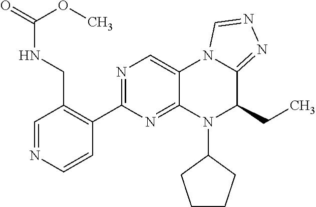 |
| 430 | 392 | | 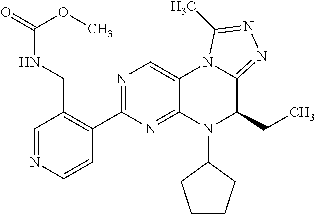 |
| 431 | 390 | 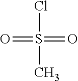 | 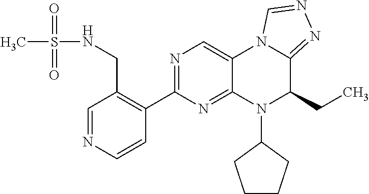 |
| 432 | 392 | | 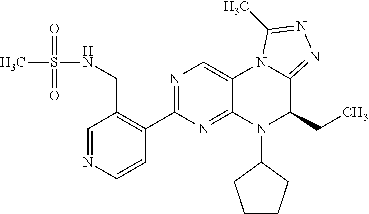 |
| 433 | 390 | 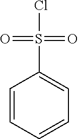 | 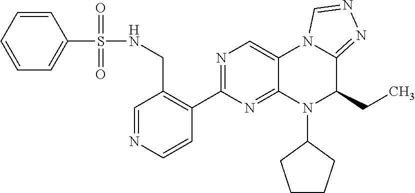 |
| 434 | 392 | | 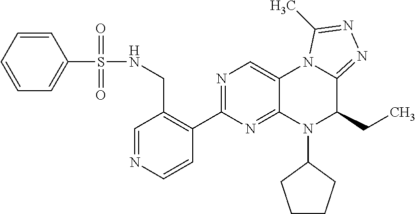 |
| 435 | 382 | 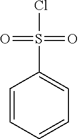 | 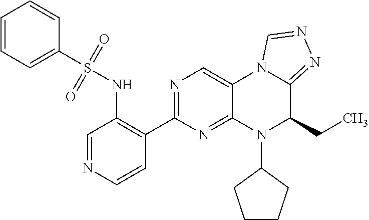 |
| 436 | 384 | | 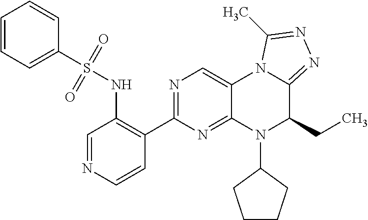 |
| 437 | 394 | 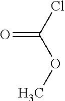 | 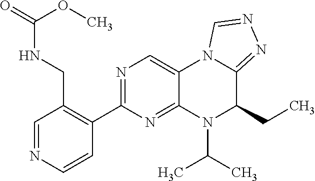 |
| 438 | 396 | | 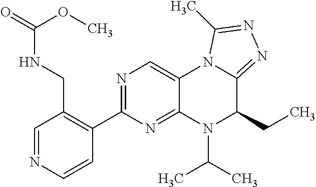 |
| 439 | 394 | 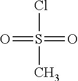 | 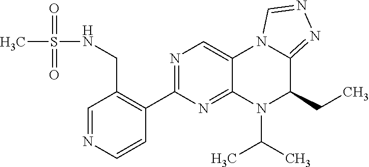 |
| 440 | 396 | | 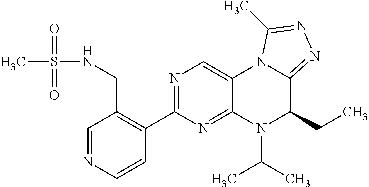 |
| 441 | 394 | 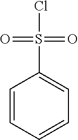 | 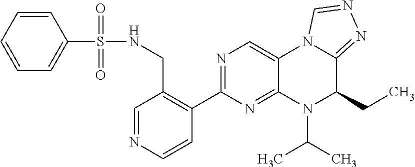 |
| 442 | 396 | | 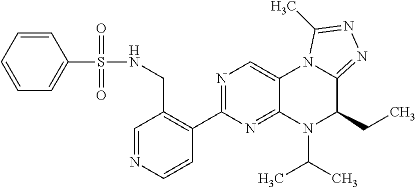 |

### Example 443

### Synthesis of (R)-5-cyclopentyl-4-ethyl-7-(4-phenylpyrimidin-5-yl)-4,5-dihydro-[1,2,4]triazolo[4,3-f]pteridine

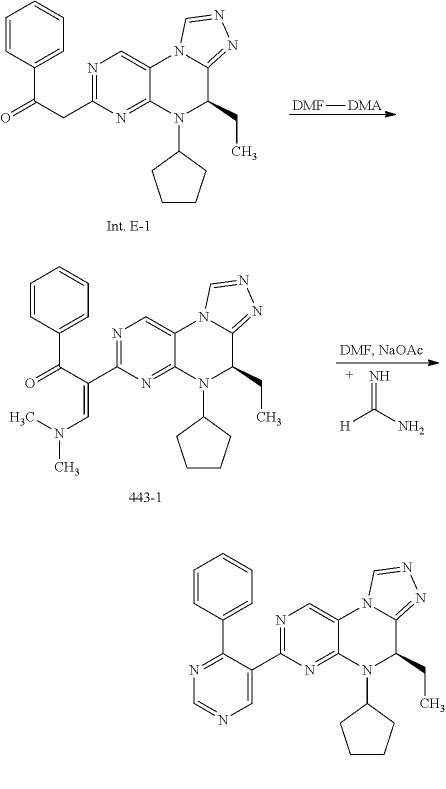

A suspension of (R)-2-(5-cyclopentyl-4-ethyl-4,5-dihydro-[1,2,4]triazolo[4,3-f]pteridin-7-yl)-1-phenylethanone (Intermediate E-1) in 10 mL of DMF-DMA is heated at 110° C. for 3 hours. The resulting mixture is concentrated to give the desired (R,Z)-2-(5-cyclopentyl-4-ethyl-4,5-dihydro-[1,2,4]triazolo[4,3 4] pteridin-7-yl)-3-(dimethylamino)-1-phenylprop-2-en-1-one (compound 443-1).

Compound 443-1 is dissolved in 5 mL of DMF, then acetate formimidamide (2.0 eq) and NaOAc (3.0 eq) are added, and the mixture is refluxed for 2 hours. The mixture is poured into ice-water, adjusted with aqueous Na₂CO₃ until PH>8, then extracted with EtOAc (3×50 mL) and purified by preparative HPLC to give the title compound.

Additional compounds are prepared similarly to this method, replacing Intermediate E-1 with a suitable Intermediate. The following compounds are prepared:
(R)-5-cyclopentyl-4-ethyl-1-methyl-7-(4-phenylpyrimidin-5-yl)-4,5-dihydro-[1,2,4]triazolo[4,3-f]pteridine (Example 444),
(R)-4-ethyl-5-isopropyl-7-(4-(4-(trifluoromethyl)phenyl)pyrimidin-5-yl)-4,5-dihydro-[1,2,4]triazolo[4,3-f]pteridine (Example 445),
(R)-4-ethyl-5-isopropyl-1-methyl-7-(4-(4-(trifluoromethyl)phenyl)pyrimidin-5-yl)-4,5-dihydro-[1,2,4]triazolo[4,3-f]pteridine (Example 446),
(R)-2-(5-(4-ethyl-5-isopropyl-4,5-dihydro-[1,2,4]triazolo[4,3-f]pteridin-7-yl)pyrimidin-4-yl)thiazole (Example 447),
(R)-2-(5-(4-ethyl-5-isopropyl-1-methyl-4,5-dihydro-[1,2,4]triazolo[4,3-f]pteridin-7-yl)pyrimidin-4-yl)thiazole (Example 448),
(S)-12a-ethyl-7-(4-phenylpyrimidin-5-yl)-10,11,12,12a-tetrahydropyrrolo[2,1-h][1,2,4]triazolo[4,3-f]pteridine (Example 449), and
(S)-12a-ethyl-3-methyl-7-(4-phenylpyrimidin-5-yl)-10,11,12,12a-tetrahydropyrrolo[2,1-h][1,2,4]triazolo[4,3-f]pteridine (Example 450).
   The following table provides the example number (column 1) and Intermediate (column 2) used to give the compound shown in column 3.

| Ex. No. | Int. | Compound structure |
|---|---|---|
| 444 | F-1 | 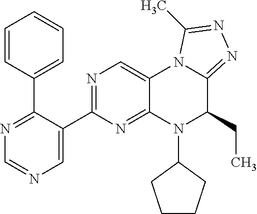 |
| 445 | G-3 | 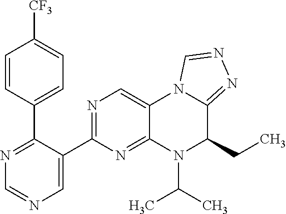 |
| 446 | H-3 | 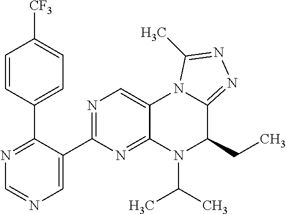 |
| 447 | G-5 | 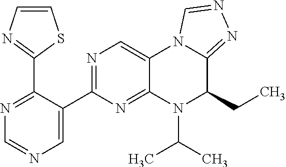 |
| 448 | H-5 | 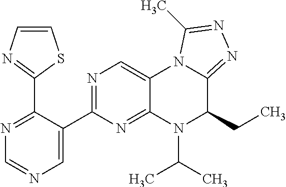 |
| 449 | XX-2 | 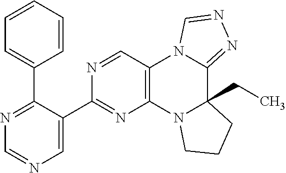 |
| 450 | XX′-2 | 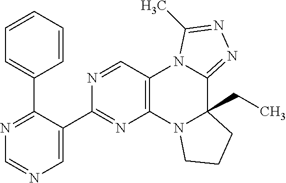 |

### Example 451

### Synthesis of (R)-4-(5-cyclopentyl-4-ethyl-4,5-dihydro-[1,2,4]triazolo[4,3-f]pteridin-7-yl)-S-phenylisoxazole

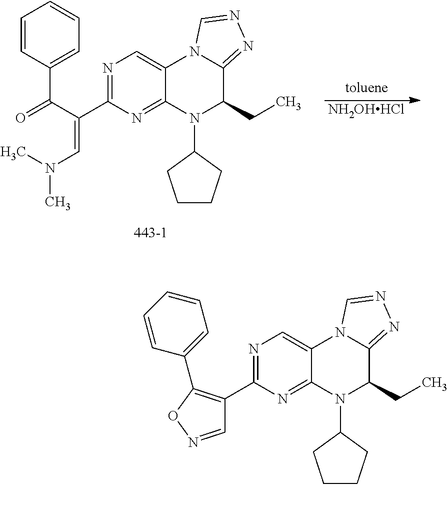

Compound 443-1 (from Example 443) is dissolved in 3 mL of toluene, then NH₂ORHCl (5.0 eq) is added and the mixture is refluxed for 2 hours. The mixture is poured into ice-water, adjusted with aqueous Na₂CO₃ until PH>8, then extracted with EtOAc (3×50 mL) and purified by silica gel column (PE:EA=3:2) to give the title compound.

Additional compounds are prepared similarly to this method, replacing compound 443-1 with a suitable analog prepared by replacing Intermediate E-1 with a suitable Intermediate in the preparation of the analogs of compound 443-1. The following compounds are prepared:
(R)-4-(5-cyclopentyl-4-ethyl-1-methyl-4,5-dihydro-[1,2,4]triazolo[4,3-f]pteridin-7-yl)-5-phenylisoxazole (Example 452),
(R)-4-(4-ethyl-5-isopropyl-4,5-dihydro-[1,2,4]triazolo[4,3-f]pteridin-7-yl)-5-(4-fluorophenyl)isoxazole (Example 453),
(R)-4-(4-ethyl-5-isopropyl-1-methyl-4,5-dihydro-[1,2,4]triazolo[4,3-f]pteridin-7-yl)-5-(4-fluorophenyl)isoxazole (Example 454),
(R)-4-(4-ethyl-5-isopropyl-4,5-dihydro-[1,2,4]triazolo[4,3-f]pteridin-7-yl)-5-(thiazol-2-yl)isoxazole (Example 455),
(R)-4-(4-ethyl-5-isopropyl-1-methyl-4,5-dihydro-[1,2,4]triazolo[4,3-f]pteridin-7-yl)-5-(thiazol-2-yl)isoxazole (Example 456),
(R)-13a-ethyl-7-(3-phenylisoxazol-4-yl)-10,11,13,13a-tetrahydro-[1,4]oxazin[3,4-h][1,2,4]triazolo[4,3-f]pteridine and (S)-13a-ethyl-3-methyl-7-(3-phenylisoxazol-4-yl)-10,11,13,13a-tetrahydro-[1,4]oxazino[3,4-h][1,2,4]triazolo[4,3-f]pteridine (Example 745),
(R)-13a-ethyl-7-(3-phenylisoxazol-4-yl)-10,11,13,13a-tetrahydro-[1,4]oxazin[3,4-h][1,2,4]triazolo[4,3-f]pteridine and (S)-13a-ethyl-3-methyl-7-(3-phenylisoxazol-4-yl)-10,11,13,13a-tetrahydro-[1,4]oxazino[3,4-h][1,2,4]triazolo[4,3-f]pteridine (Example 746).
   In the case of a racemic mixture, the isomers can be isolated by chiral chromatography. The following table provides the example number (column 1) and Intermediate (column 2) used in preparation of analogs of compound 443-1 to give the compound shown in column 3.

| Ex. No. | Int. | Compound structure |
|---|---|---|
| 452 | F-1 | 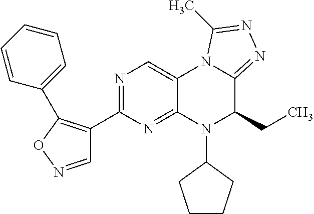 |
| 453 | G-4 | 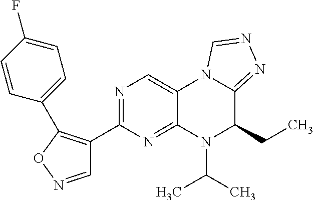 |
| 454 | H-4 | 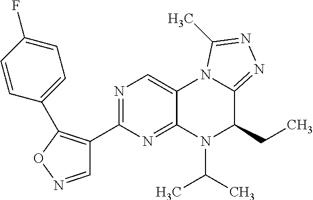 |
| 455 | G-5 | 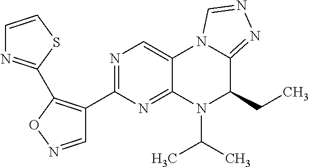 |
| 456 | H-5 | 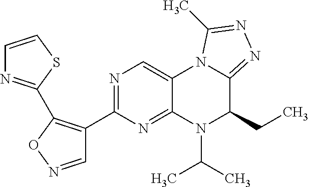 |
| 745(R) | Z-2 | 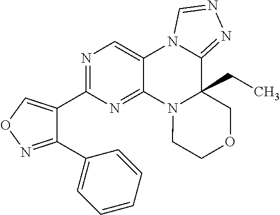 |
| 745(S) | | 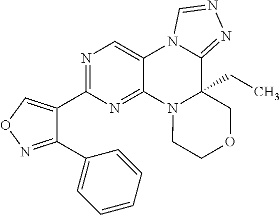 |
| 746(R) | Z′-2 | 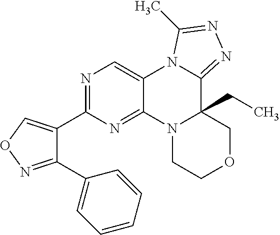 |
| 746(S) | | 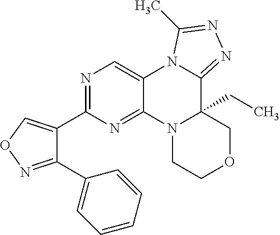 |

### Example 457

### Synthesis of (R)-5-cyclopentyl-4-ethyl-7-(5-phenyl-1H-pyrazol-4-yl)-4,5-dihydro-[1,2,4]triazolo[4,3-f]pteridine

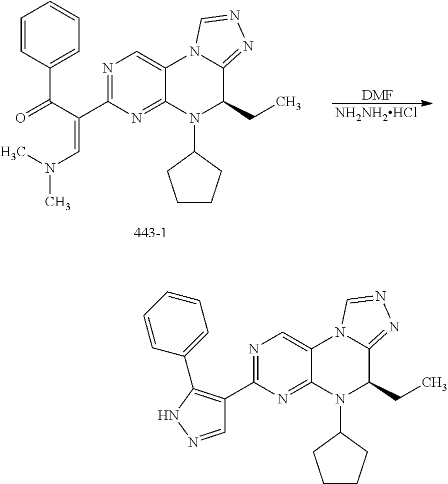

Compound 443-1 (from Example 443) is dissolved in 5 mL of DMF, then NH₂NH₂.HCl (3.0 eq) is added and the mixture is refluxed for 2 hours. The mixture is poured into ice-water, adjusted with aqueous Na₂CO₃ until PH>8, then extracted with EtOAc (3×50 mL) and purified by preparative HPLC to give the title compound.

Additional compounds are prepared similarly to this method, replacing compound 443-1 with a suitable analog prepared by replacing Intermediate E-1 with a suitable Intermediate in the preparation of the analogs of compound 443-1. In some instances, where a racemic mixture results, the two enantiomers may be isolated by chiral chromatography. The following compounds are prepared:
(R)-5-cyclopentyl-4-ethyl-1-methyl-7-(5-phenyl-1H-pyrazol-4-yl)-4,5-dihydro-[1,2,4]triazolo[4,3-f]pteridine (Example 458),
(R)-4-ethyl-5-isopropyl-7-(5-phenyl-1H-pyrazol-4-yl)-4,5-dihydro-[1,2,4]triazolo[4,3-f]pteridine (Example 459),
(R)-4-ethyl-5-isopropyl-1-methyl-7-(5-phenyl-1H-pyrazol-4-yl)-4,5-dihydro-[1,2,4]triazolo[4,3-f]pteridine (Example 460),
(R)-4-ethyl-5-isopropyl-7-(5-(4-(trifluoromethyl)phenyl)-1H-pyrazol-4-yl)-4,5-dihydro-[1,2,4]triazolo[4,3-f]pteridine (Example 461),
(R)-4-ethyl-5-isopropyl-1-methyl-7-(5-(4-(trifluoromethyl)phenyl)-1H-pyrazol-4-yl)-4,5-dihydro-[1,2,4]triazolo[4,3-f]pteridine (Example 462),
(R)-2-(4-(4-ethyl-5-isopropyl-4,5-dihydro-[1,2,4]triazolo[4,3-f]pteridin-7-yl)-1H-pyrazol-3-yl)thiazole (Example 463),
(R)-2-(4-(4-ethyl-5-isopropyl-1-methyl-4,5-dihydro-[1,2,4]triazolo[4,3-f]pteridin-7-yl)-1H-pyrazol-3-yl)thiazole (Example 464),
(S)-12a-ethyl-7-(5-phenyl-1H-pyrazol-4-yl)-10,11,12,12a-tetrahydropyrrolo[2,1-h][1,2,4]triazolo[4,3-f]pteridine (Example 465),
(S)-12a-ethyl-3-methyl-7-(5-phenyl-1H-pyrazol-4-yl)-10,11,12,12a-tetrahydropyrrolo[2,1-h][1,2,4]triazolo[4,3-f]pteridine (Example 466),
(S)-2-(4-(12a-ethyl-10,11,12,12a-tetrahydropyrrolo[2,1-h][1,2,4]triazolo[4,3-f]pteridin-7-yl)-1H-pyrazol-5-yl)thiazole (Example 467),
(S)-2-(4-(12a-ethyl-3-methyl-10,11,12,12a-tetrahydropyrrolo[2,1-h][1,2,4]triazolo[4,3-f]pteridin-7-yl)-1H-pyrazol-5-yl)thiazole (Example 468),
(R)-13a-ethyl-7-(5-(thiazol-2-yl)-1H-pyrazol-4-yl)-10,11,13,13a-tetrahydro-[1,4]oxazino[3,4-h][1,2,4]triazolo[4,3-f]pteridine and (S)-13a-ethyl-7-(5-(thiazol-2-yl)-1H-pyrazol-4-yl)-10,11,13,13a-tetrahydro-[1,4]oxazino[3,4-h][1,2,4]triazolo[4,3-f]pteridine (Example 469), and
(R)-13a-ethyl-3-methyl-7-(5-(thiazol-2-yl)-1H-pyrazol-4-yl)-10,11,13,13a-tetrahydro-[1,4]oxazino[3,4-h][1,2,4]triazolo[4,3-f]pteridine and (S)-13a-ethyl-3-methyl-7-(5-(thiazol-2-yl)-1H-pyrazol-4-yl)-10,11,13,13a-tetrahydro-[1,4]oxazino[3,4-h][1,2,4]triazolo[4,3-f]pteridine (Example 470),
(R)-7-(5-(2,4-difluorophenyl)-1H-pyrazol-4-yl)-13a-ethyl-10,11,13,13a-tetrahydro-[1,4]oxazino[3,4-h][1,2,4]triazolo[4,3-f]pteridine and (S)-7-(5-(2,4-difluorophenyl)-1H-pyrazol-4-yl)-13a-ethyl-10,11,13,13a-tetrahydro-[1,4]oxazino[3,4-h][1,2,4]triazolo[4,3-f]pteridine (Example 733), and
(R)-7-(5-(2,4-difluorophenyl)-1H-pyrazol-4-yl)-13a-ethyl-3-methyl-10,11,13,13a-tetrahydro-[1,4]oxazino[3,4-h][1,2,4]triazolo[4,3-f]pteridine and (S)-7-(5-(2,4-difluorophenyl)-1H-pyrazol-4-yl)-13a-ethyl-3-methyl-10,11,13,13a-tetrahydro-[1,4]oxazino[3,4-h][1,2,4]triazolo[4,3-f]pteridine (Example 734).
   The following table provides the example number (column 1) and Intermediate (column 2) used in preparation of analogs of compound 443-1 to give the compound shown in column 3.

| Ex. No. | Int. | Compound structure |
|---|---|---|
| 458 | F-1 | 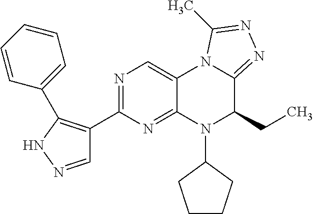 |
| 459 | G-2 | 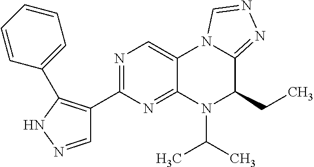 |
| 460 | H-2 | 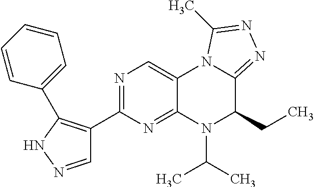 |
| 461 | G-3 | 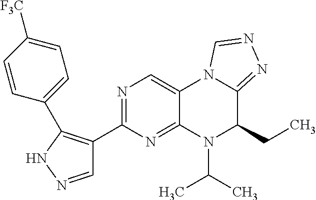 |
| 462 | H-3 | 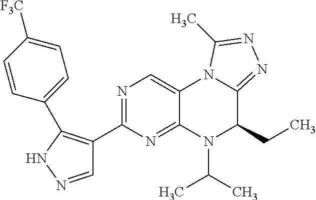 |
| 463 | G-5 | 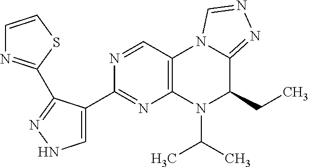 |
| 464 | H-5 | 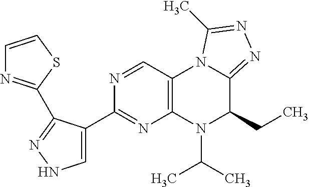 |
| 465 | XX-2 | 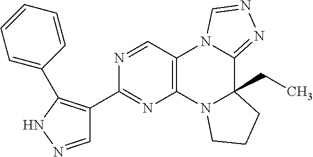 |
| 466 | XX′-2 | 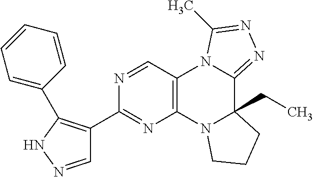 |
| 467 | XX-3 | 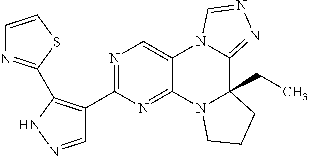 |
| 468 | XX′-3 | 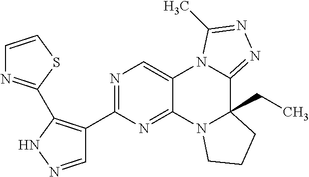 |
| 469(R) | Z-3 | 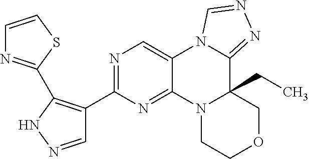 |
| 469(S) | | 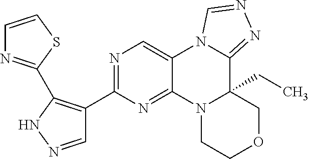 |
| 470(R) | Z′-3 | 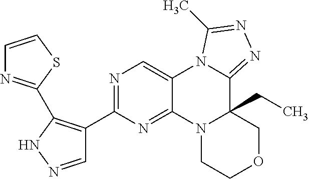 |
| 470(S) | | 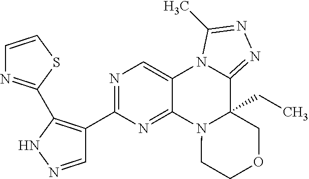 |
| 733(R) | Z-4 | 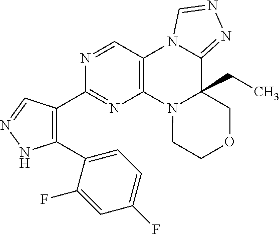 |
| 733(S) | | 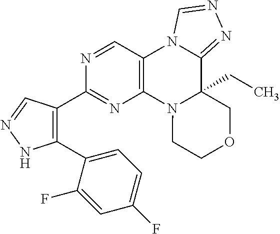 |
| 734(R) | Z′-4 | 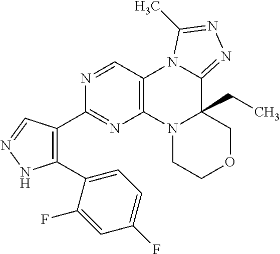 |
| 734(S) | | 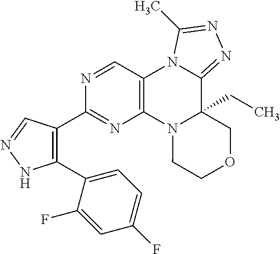 |

### Example 471

### Synthesis of (R)-5-cyclopentyl-4-ethyl-7-(5-phenyl-1H-1,2,4-triazol-1-yl)-4,5-dihydro-[1,2,4]triazolo[4,3-f]pteridine (471) and (R)-5-cyclopentyl-4-ethyl-1-methyl-7-(5-phenyl-1H-1,2,4-triazol-1-yl)-4,5-dihydro-[1,2,4]triazolo[4,3-f]pteridine (472)

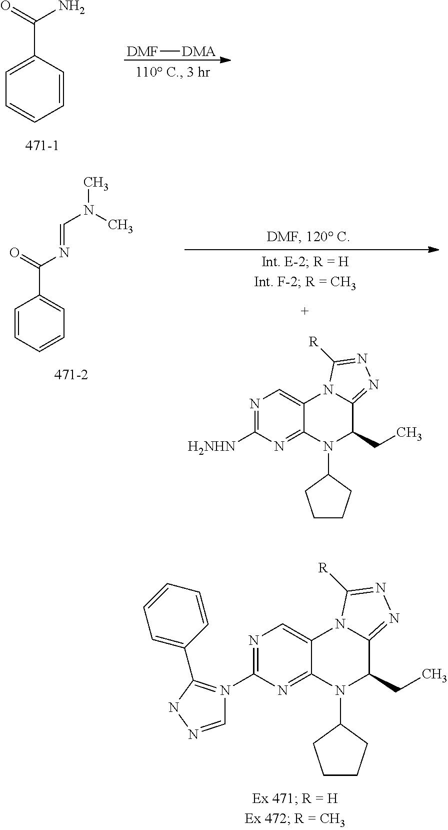

A solution of benzamide (471-1) in DMF-DMA was stirred for 3 h at 110° C., then cooled to rt. The solid was collected by filtration, the filter cake washed with PE and air dried to give the desired (E)-N-((dimethylamino)methylene)benzamide (compound 471-2).

Compound 471-2 (1.5 eq) and Intermediate E-2 or Intermediate F-2 (1 eq) in DMF is stirred for 3 h at 110° C. The mixture is cooled to rt, diluted with water and extracted with EtOAc. The organic layer is dried with Na₂SO₄, concentrated and the residue is purified by silica gel column chromatography to give the title compounds.
(R)-7-(5-(1H-pyrazol-5-yl)-1H-1,2,4-triazol-1-yl)-4-ethyl-5-isopropyl-4,5-dihydro-[1,2,4]triazolo[4,3-f]pteridine (Example 473) and (R)-7-(5-(1H-pyrazol-5-yl)-1H-1,2,4-triazol-1-yl)-4-ethyl-5-isopropyl-1-methyl-4,5-dihydro-[1,2,4]triazolo[4,3-f]pteridine (Example 474),

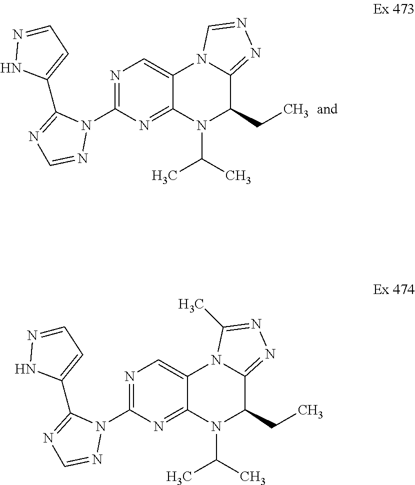

are prepared similarly, with 1H-pyrazole-5-carboxamide instead of benzamide in the first step, and with Intermediate G-6 or H-6 instead of Intermediate E-2 or F-2, respectively, in the last step.

### Example 475

### Synthesis of (R)-5-cyclopentyl-4-ethyl-7-(3-phenylpyrazin-2-yl)-4,5-dihydro-[1,2,4]triazolo[4,3-f]pteridine

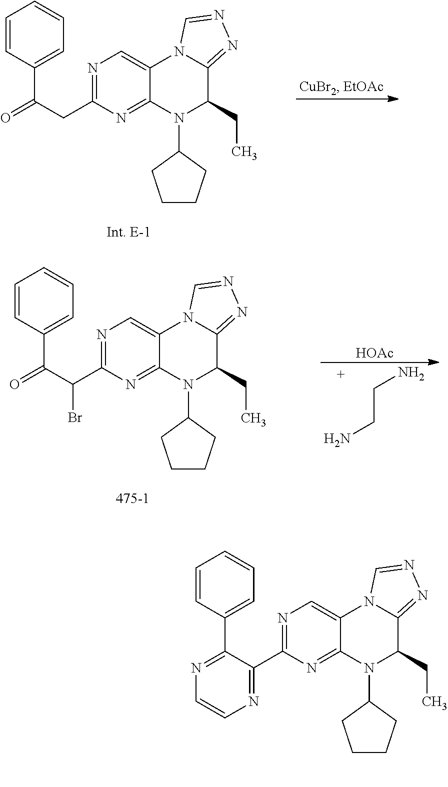

To a solution of (R)-2-(5-cyclopentyl-4-ethyl-4,5-dihydro-[1,2,4]triazolo[4,3-f]pteridin-7-yl)-1-phenylethanone (Intermediate E-1) in EtOAc, CuBr₂ (10.0 eq) is added and the reaction is stirred at reflux state for 1.5 hours. The mixture is filtered and water is added to the filtrate, adjusted PH>8 with Na₂CO₃ aqueous, extracted 3× with EtOAc, and concentrated to give the desired 2-bromo-2-((R)-5-cyclopentyl-4-ethyl-4,5-dihydro-[1,2,4]triazolo[4,3-f]pteridin-7-yl)-1-phenylethanone (compound 475-1).

Compound 475-1 is dissolved in HOAc, then ethane-1,2-diamine is added and the mixture is refluxed for 5 hours in open air. The mixture s poured into ice-water, adjusted with aqueous Na₂CO₃ until PH>9, then extracted 3× with EtOAc and purified by preparative HPLC to give the title compound.

Additional compounds are prepared similarly to this method, replacing Intermediate E-1 with a suitable Intermediate. The following compounds are prepared:
(R)-5-cyclopentyl-4-ethyl-1-methyl-7-(3-phenylpyrazin-2-yl)-4,5-dihydro-[1,2,4]triazolo[4,3-f]pteridine (Example 476),
(R)-4-ethyl-5-isopropyl-7-(3-(4-(trifluoromethyl)phenyl)pyrazin-2-yl)-4,5-dihydro-[1,2,4]triazolo[4,3-f]pteridine (Example 477),
(R)-4-ethyl-5-isopropyl-1-methyl-7-(3-(4-(trifluoromethyl)phenyl)pyrazin-2-yl)-4,5-dihydro-[1,2,4]triazolo[4,3-f]pteridine (Example 478),
(R)-4-ethyl-7-(3-(4-fluorophenyl)pyrazin-2-yl)-5-isopropyl-4,5-dihydro-[1,2,4]triazolo[4,3-f]pteridine (Example 479),
(R)-4-ethyl-7-(3-(4-fluorophenyl)pyrazin-2-yl)-5-isopropyl-1-methyl-4,5-dihydro-[1,2,4]triazolo[4,3-f]pteridine (Example 480),
(S)-12a-ethyl-7-(3-phenylpyrazin-2-yl)-10,11,12,12a-tetrahydropyrrolo[2,1-h][1,2,4]triazolo[4,3-f]pteridine (Example 481), and
(S)-12a-ethyl-3-methyl-7-(3-phenylpyrazin-2-yl)-10,11,12,12a-tetrahydropyrrolo[2,1-h][1,2,4]triazolo[4,3-f]pteridine (Example 482).
   The following table provides the example number (column 1) and Intermediate (column 2) used to give the compound shown in column 3.

| Ex. No. | Int. | Compound structure |
|---|---|---|
| 476 | F-1 | 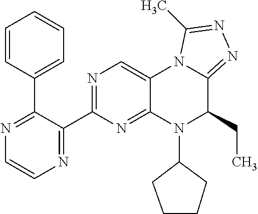 |
| 477 | G-3 | 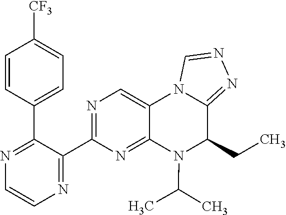 |
| 478 | H-3 | 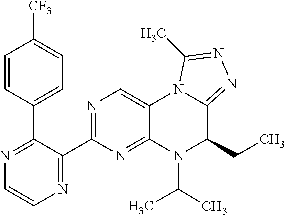 |
| 479 | G-4 | 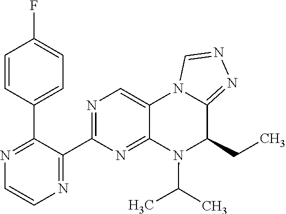 |
| 480 | H-4 | 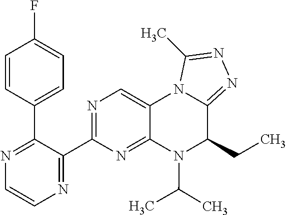 |
| 481 | XX-2 | 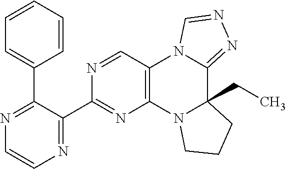 |
| 482 | XX′-2 | 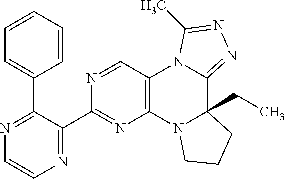 |

### Example 483 and Example 484

### Synthesis of (R)-5-cyclopentyl-4-ethyl-7-(5-phenylpyridazin-4-yl)-4,5-dihydro-[1,2,4]triazolo[4,3-f]pteridine (483) and (R)-5-cyclopentyl-4-ethyl-1-methyl-7-(5-phenylpyridazin-4-yl)-4,5-dihydro-[1,2,4]triazolo[4,3-f]pteridine (484)

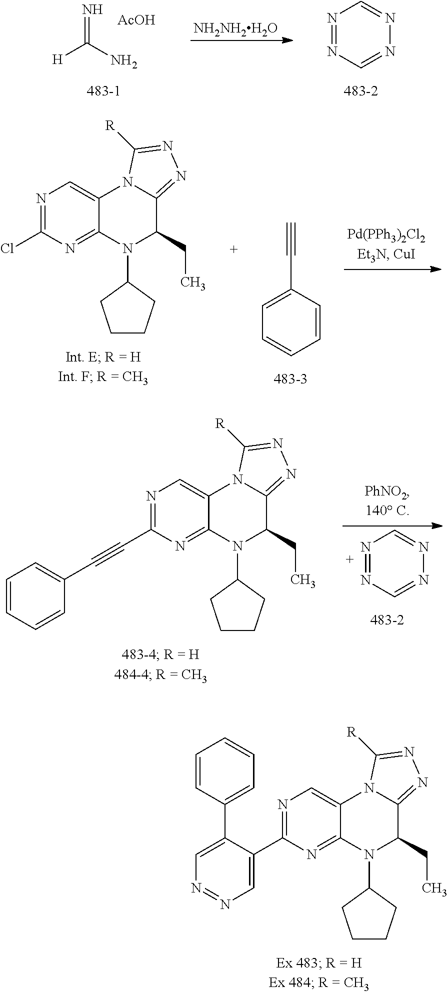

To the acetate of formimidamide (compound 483-1, 3.12 g, 0.03 mol) cooled in ice, 4 ml of hydrazine hydrate (0.08 mol) was added slowly. The resulting mixture was stirred for 1 hour at rt. After addition of 2 ml of water and stirring at 0° C. for 1 hour, the precipitate was filtered off. The precipitate was dissolved in 10 mL of acetic acid and 1 g of sodium nitrite was added in small portions at about 5° C. After stirring for 1 hour, 15 mL of water was added and the mixture was extracted with DCM (4×15 mL). The combined DCM layers were washed with aqueous NaHCO₃ until neutralized, dried with MgSO₄ and concentrated to give 1,2,4,5-tetrazine (compound 483-2) as a red solid.

To a solution of Intermediate E or Intermediate F (1.0 eq) in DMF, ethynylbenzene (compound 483-3, 3.0 eq), Pd(PPh₃)₂Cl₂ (0.2 eq), CuI (0.25 eq) and Et₃N (5.0 eq) are added. The mixture is refluxed for 18 h under argon, extracted with EtOAc and purified by silica gel column to give compound 483-4 or 484-4.

Compound 483-2 (2.0 eq) and compound 483-4 or 484-4 (1.0 eq) are combined with nitrobenzene in a sealed tube and heated to 140° C. for 3 hours. Solvent is removed under reduced pressure and the residue is purified by reverse phase HPLC to give the title compounds.

### Example 485 and Example 486

### Synthesis of 4-perdeuteroethyl-5-perdeutoeroisopropyl-7-(3-phenyl-1H-pyrazol-4-yl)-4,5-dihydro-[1,2,4]triazolo[4,3-f]pteridine (485) and 4-perdeuteroethyl-5-perdeutoeroisopropyl-1-methyl-7-(3-phenyl-1H-pyrazol-4-yl)-4,5-dihydro-[1,2,4]triazolo[4,3-f]pteridine (486)

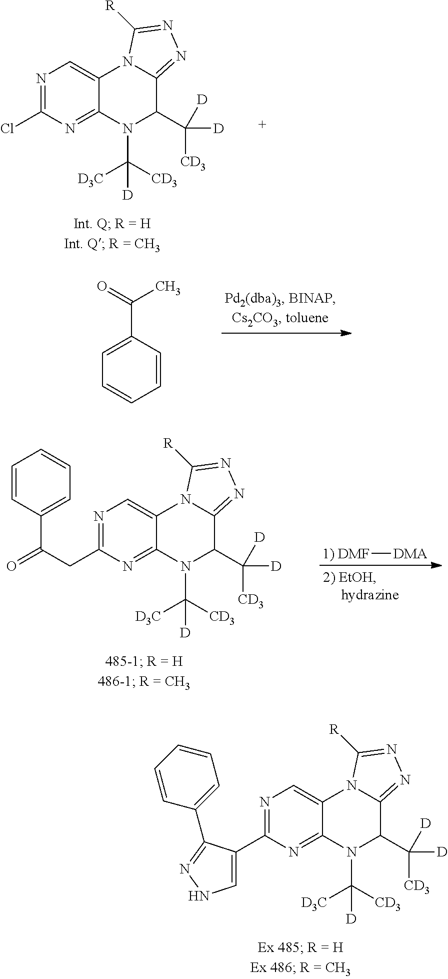

To a stirring mixture of Intermediate Q or Intermediate Q′ (1 eq) in toluene/water (1.0/0.2), Pd₂(dba)₃ (0.2 eq), BINAP (0.4 eq), acetophenone (3 eq), and Cs₂CO₃ (3 eq) are added. The reaction mixture is heated in a microwave at 140° C. for 1 h. The crude mixture is purified by MPLC to provide compound 485-1 or 486-1.

Compound 485-1 or 486-1 (1 eq) is dissolved in N,N-Dimethylformamide dimethyl acetal (15 eq). The reaction mixture is stirred at 80° C. for 2 h. The crude mixture is concentrated under reduced pressure and dissolved in EtOH, then hydrazine is added. The reaction mixture is warmed to 78° C. for 1 h. The crude reaction mixture is purified by preperative HPLC to give the title compounds.

### Example 487

### Synthesis of (R)-7-(2-(3,5-dichlorophenyl)-1H-imidazol-1-yl)-4-ethyl-5-isopropyl-4,5-dihydro-[1,2,4]triazolo[4,3-f]pteridine

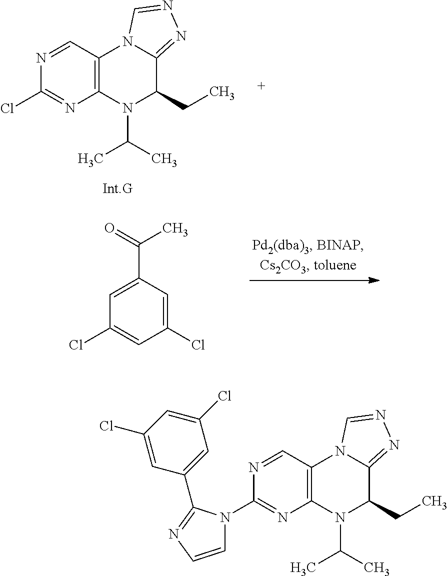

A 5 mL microwave vial is charged with Intermediate G (0.19 mmol), 2-(3,5-dichlorophenyl)-1H-imidazole (0.37 mmol), Pd₂(dba)₃ (0.04 mmol), BINAP (0.08 mmol), Cs₂CO₃ (0.37 mmol), and dioxane. The vial is sealed and heated in a microwave to 150° C. for 0.5 h. Upon cooling to 23° C., the reaction mix is diluted with EtOAc, and rinsed sequentially with saturated aqueous solutions of ammonium chloride, sodium bicarbonate, and brine. The resulting organic liquid is dried over sodium sulfate and decanted into a round bottom flask and concentrated, and the resulting residue is purified by HPLC to give the title compound.

Additional compounds are prepared similarly to this method, optionally replacing Intermediate G with a suitable Intermediate and/or replacting 2-(3,5-dichlorophenyl)-1H-imidazole with a suitable ring reagent. In some instances, where a racemic mixture results, the two enantiomers may be isolated by chiral chromatography.

The following compounds are prepared:
(R)-7-(2-(3,5-dichlorophenyl)-1H-imidazol-1-yl)-4-ethyl-5-isopropyl-1-methyl-4,5-dihydro-[1,2,4]triazolo[4,3-f]pteridine (Example 488),
(R)-5-cyclobutyl-7-(2-(3,5-dichlorophenyl)-1H-imidazol-1-yl)-4-ethyl-4,5-dihydro-[1,2,4]triazolo[4,3-f]pteridine (Example 489),
(R)-5-cyclobutyl-7-(2-(3,5-dichlorophenyl)-1H-imidazol-1-yl)-4-ethyl-1-methyl-4,5-dihydro-[1,2,4]triazolo[4,3-f]pteridine (Example 490),
(S)-13a-ethyl-7-(2-phenyl-1H-imidazol-1-yl)-11,12,13,13a-tetrahydro-10H-pyrido[2,1-h][1,2,4]triazolo[4,3-f]pteridine and (R)-13a-ethyl-7-(2-phenyl-1H-imidazol-1-yl)-11,12,13,13a-tetrahydro-10H-pyrido[2,1-h][1,2,4]triazolo[4,3-f]pteridine (Example 491),
(S)-13a-ethyl-3-methyl-7-(2-phenyl-1H-imidazol-1-yl)-11,12,13,13a-tetrahydro-10H-pyrido[2,1-h][1,2,4]triazolo[4,3-f]pteridine and (R)-13a-ethyl-3-methyl-7-(2-phenyl-1H-imidazol-1-yl)-11,12,13,13a-tetrahydro-10H-pyrido[2,1-h][1,2,4]triazolo[4,3-f]pteridine (Example 492),
(S)-13a-ethyl-7-(2-(3-fluorophenyl)-1H-imidazol-1-yl)-11,12,13,13a-tetrahydro-10H-pyrido[2,1-h][1,2,4]triazolo[4,3-f]pteridine and (R)-13a-ethyl-7-(2-(3-fluorophenyl)-1H-imidazol-1-yl)-11,12,13,13a-tetrahydro-10H-pyrido[2,1-h][1,2,4]triazolo[4,3-f]pteridine (Example 493),
(S)-13a-ethyl-7-(2-(3-fluorophenyl)-1H-imidazol-1-yl)-3-methyl-11,12,13,13a-tetrahydro-10H-pyrido[2,1-h][1,2,4]triazolo[4,3-f]pteridine and (R)-13a-ethyl-7-(2-(3-fluorophenyl)-1H-imidazol-1-yl)-3-methyl-11,12,13,13a-tetrahydro-10H-pyrido[2,1-h][1,2,4]triazolo[4,3-f]pteridine (Example 494),
7-(2-(3,4-difluorophenyl)-1H-imidazol-1-yl)-13a-ethyl-11,12,13,13a-tetrahydro-10H-pyrido[2,1-h][1,2,4]triazolo[4,3-f]pteridine (Example 495),
7-(2-(3,4-difluorophenyl)-1H-imidazol-1-yl)-13a-ethyl-3-methyl-11,12,13,13a-tetrahydro-10H-pyrido[2,1-h][1,2,4]triazolo[4,3-f]pteridine (Example 496),
(S)-7-(2-(3,5-difluorophenyl)-1H-imidazol-1-yl)-13a-ethyl-11,12,13,13a-tetrahydro-10H-pyrido[2,1-h][1,2,4]triazolo[4,3-f]pteridine and (R)-7-(2-(3,5-difluorophenyl)-1H-imidazol-1-yl)-13a-ethyl-11,12,13,13a-tetrahydro-10H-pyrido[2,1-h][1,2,4]triazolo[4,3-f]pteridine (Example 497),
(S)-7-(2-(3,5-difluorophenyl)-1H-imidazol-1-yl)-13a-ethyl-3-methyl-11,12,13,13a-tetrahydro-10H-pyrido[2,1-h][1,2,4]triazolo[4,3-f]pteridine and (R)-7-(2-(3,5-difluorophenyl)-1H-imidazol-1-yl)-13a-ethyl-3-methyl-11,12,13,13a-tetrahydro-10H-pyrido[2,1-h][1,2,4]triazolo[4,3-f]pteridine (Example 498),
(S)-7-(2-(2,3-difluorophenyl)-1H-imidazol-1-yl)-13a-ethyl-11,12,13,13a-tetrahydro-10H-pyrido[2,1-h][1,2,4]triazolo[4,3-f]pteridine and (R)-7-(2-(2,3-difluorophenyl)-1H-imidazol-1-yl)-13a-ethyl-11,12,13,13a-tetrahydro-10H-pyrido[2,1-h][1,2,4]triazolo[4,3-f]pteridine (Example 499),
(S)-7-(2-(2,3-difluorophenyl)-1H-imidazol-1-yl)-13a-ethyl-3-methyl-11,12,13,13a-tetrahydro-10H-pyrido[2,1-h][1,2,4]triazolo[4,3-f]pteridine and (R)-7-(2-(2,3-difluorophenyl)-1H-imidazol-1-yl)-13a-ethyl-3-methyl-11,12,13,13a-tetrahydro-10H-pyrido[2,1-h][1,2,4]triazolo[4,3-f]pteridine (Example 500),
(S)-13a-ethyl-7-(2-(3,4,5-trifluorophenyl)-1H-imidazol-1-yl)-11,12,13,13a-tetrahydro-10H-pyrido[2,1-h][1,2,4]triazolo[4,3-f]pteridine and (R)-13a-ethyl-7-(2-(3,4,5-trifluorophenyl)-1H-imidazol-1-yl)-11,12,13,13a-tetrahydro-10H-pyrido[2,1-h][1,2,4]triazolo[4,3-f]pteridine (Example 501),
(S)-13a-ethyl-3-methyl-7-(2-(3,4,5-trifluorophenyl)-1H-imidazol-1-yl)-11,12,13,13a-tetrahydro-10H-pyrido[2,1-h][1,2,4]triazolo[4,3-f]pteridine and (R)-13a-ethyl-3-methyl-7-(2-(3,4,5-trifluorophenyl)-1H-imidazol-1-yl)-11,12,13,13a-tetrahydro-10H-pyrido[2,1-h][1,2,4]triazolo[4,3-f]pteridine (Example 502),
(S)-7-(2-(2,4-difluorophenyl)-1H-imidazol-1-yl)-13a-ethyl-11,12,13,13a-tetrahydro-10H-pyrido[2,1-h][1,2,4]triazolo[4,3-f]pteridine and (R)-7-(2-(2,4-difluorophenyl)-1H-imidazol-1-yl)-13a-ethyl-11,12,13,13a-tetrahydro-10H-pyrido[2,1-h][1,2,4]triazolo[4,3-f]pteridine (Example 503),
(S)-7-(2-(2,4-difluorophenyl)-1H-imidazol-1-yl)-13a-ethyl-3-methyl-11,12,13,13a-tetrahydro-10H-pyrido[2,1-h][1,2,4]triazolo[4,3-f]pteridine and (R)-7-(2-(2,4-difluorophenyl)-1H-imidazol-1-yl)-13a-ethyl-3-methyl-11,12,13,13a-tetrahydro-10H-pyrido[2,1-h][1,2,4]triazolo[4,3-f]pteridine (Example 504),
(S)-12a-ethyl-7-(2-phenyl-4,5-dihydro-1H-imidazol-1-yl)-10,11,12,12a-tetrahydropyrrolo[2,1-h][1,2,4]triazolo[4,3-f]pteridine (Example 505),
(S)-12a-ethyl-3-methyl-7-(2-phenyl-4,5-dihydro-1H-imidazol-1-yl)-10,11,12,12a-tetrahydropyrrolo[2,1-h][1,2,4]triazolo[4,3-f]pteridine (Example 506),
(S)-13a-ethyl-7-(1H-imidazol-1-yl)-11,12,13,13a-tetrahydro-10H-pyrido[2,1-h][1,2,4]triazolo[4,3-f]pteridine and (R)-13a-ethyl-7-(1H-imidazol-1-yl)-11,12,13,13a-tetrahydro-10H-pyrido[2,1-h][1,2,4]triazolo[4,3-f]pteridine (Example 507),
(S)-13a-ethyl-7-(1H-imidazol-1-yl)-3-methyl-11,12,13,13a-tetrahydro-10H-pyrido[2,1-h][1,2,4]triazolo[4,3-f]pteridine and (R)-13a-ethyl-7-(1H-imidazol-1-yl)-3-methyl-11,12,13,13a-tetrahydro-10H-pyrido[2,1-h][1,2,4]triazolo[4,3-f]pteridine (Example 508),
(R)-7-(2-chloro-1H-imidazol-1-yl)-5-cyclopentyl-4-ethyl-4,5-dihydro-[1,2,4]triazolo[4,3-f]pteridine (Example 509), and
(R)-7-(2-chloro-1H-imidazol-1-yl)-5-cyclopentyl-4-ethyl-1-methyl-4,5-dihydro-[1,2,4]triazolo[4,3-f]pteridine (Example 510).

The following table provides the example number (column 1), Intermediate (column 2), and ring reactant (column 3) used to give the compound shown in column 4.

| Ex. No. | Int. | Ring reactant | Compound structure |
|---|---|---|---|
| 488 | H | 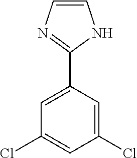 | 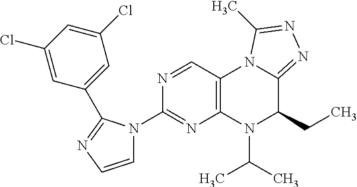 |
| 489 | C | 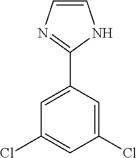 | 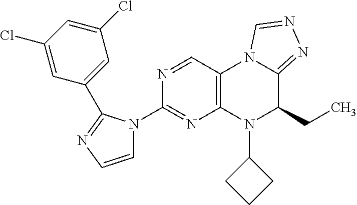 |
| 490 | D | | 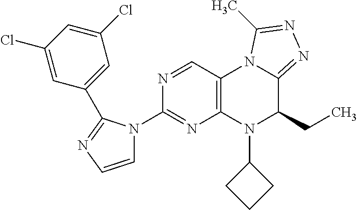 |
| 491(S) | Y | 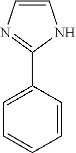 | 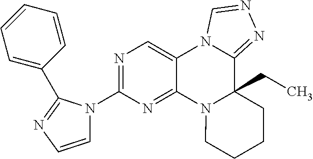 |
| 491(R) | | | 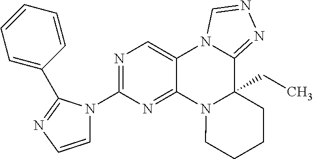 |
| 492(S) | Y′ | 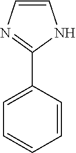 | 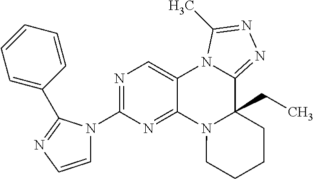 |
| 492(R) | | | 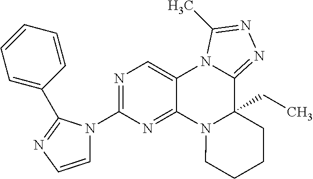 |
| 493(S) | Y | 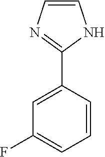 | 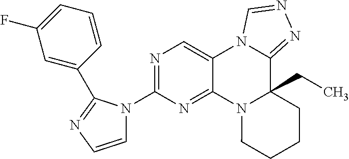 |
| 493(R) | | | 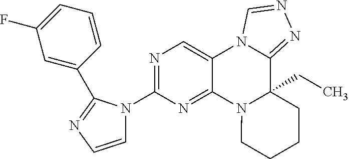 |
| 494(S) | Y′ | 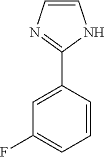 | 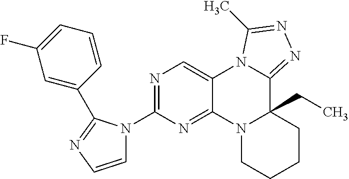 |
| 494(R) | | | 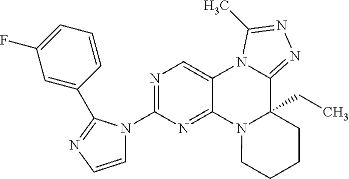 |
| 495 | Y | 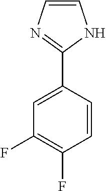 | 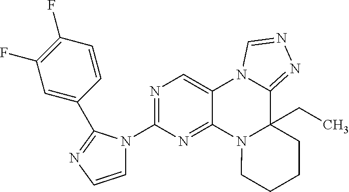 |
| 496 | Y′ | | 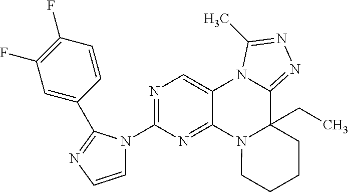 |
| 497(S) | Y | 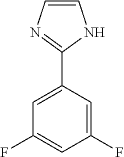 | 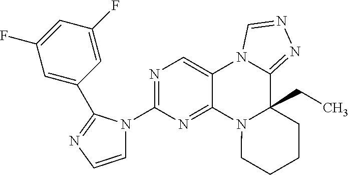 |
| 497(R) | | | 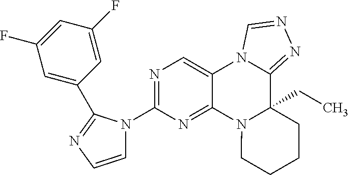 |
| 498(S) | Y′ | 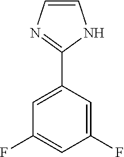 | 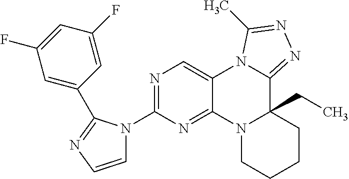 |
| 498(R) | | | 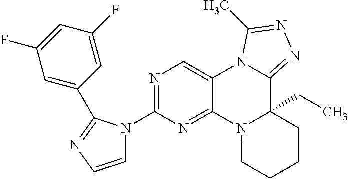 |
| 499(S) | Y | 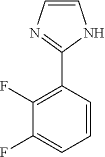 | 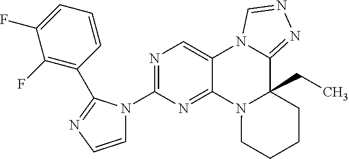 |
| 499(R) | | | 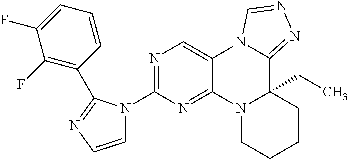 |
| 500(S) | Y′ | 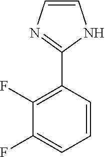 | 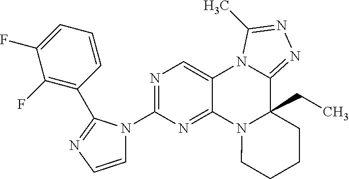 |
| 500(R) | | | 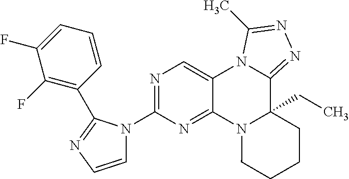 |
| 501(S) | Y | 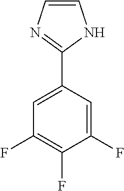 | 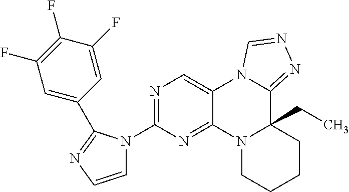 |
| 501(R) | | | 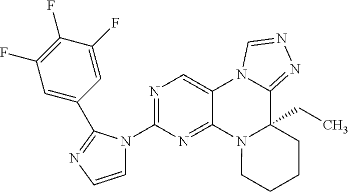 |
| 502(S) | Y′ | 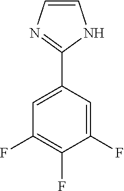 | 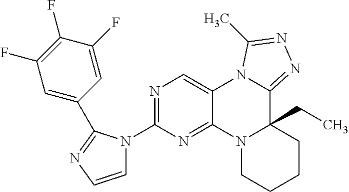 |
| 502(R) | | | 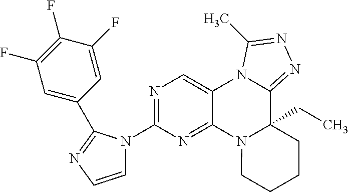 |
| 503(S) | Y | 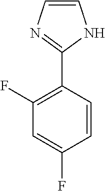 | 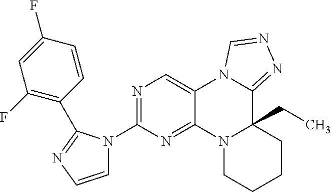 |
| 503(R) | | | 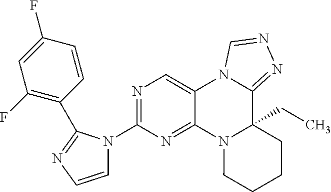 |
| 504(S) | Y′ | 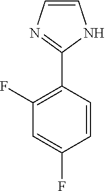 | 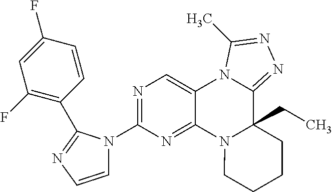 |
| 504(R) | | | 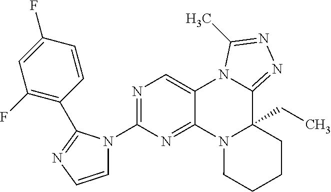 |
| 505 | XX | 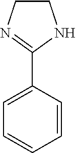 | 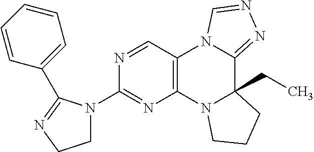 |
| 506 | XX′ | | 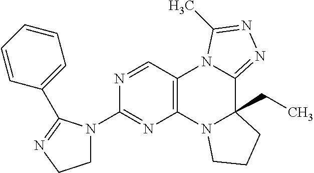 |
| 507(S) | Y | 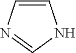 | 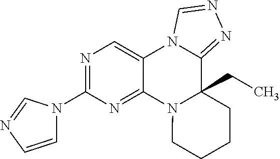 |
| 507(R) | | | 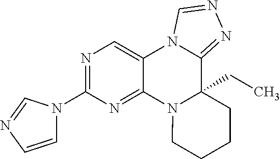 |
| 508(S) | Y′ | 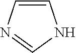 | 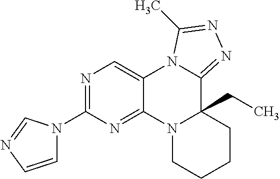 |
| 508(R) | | | 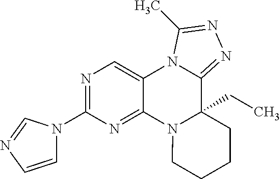 |
| 509 | E | 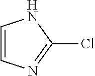 | 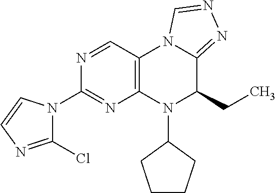 |
| 510 | F | | 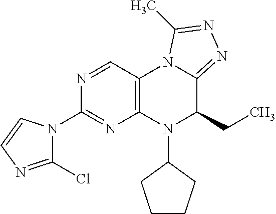 |

### Example 511

### Synthesis of (R)-4-ethyl-5-isopropyl-7-(5-phenyl-1H-1,2,4-triazol-1-yl)-4,5-dihydro-[1,2,4]triazolo[4,3-f]pteridine

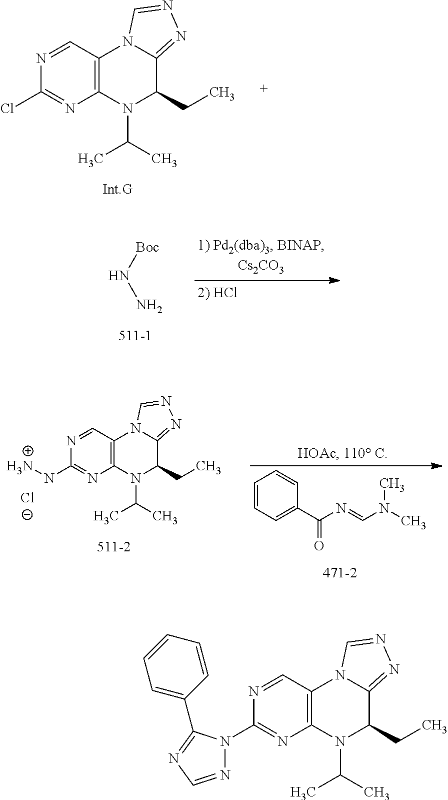

A 5 mL microwave vial is charged with Intermediate G (0.56 mmol), tert-butyl hydrazinecarboxylate (511-1, 1.68 mmol), Pd₂(dba)₃ (0.12 mmol), BINAP (0.24 mmol), Cs₂CO₃ (1.68 mmol), and dioxane. The vial is sealed and heated in a microwave to 150° C. for 0.5 h. Upon cooling to 23° C., the reaction mix is diluted with EtOAc, and rinsed sequentially with saturated, aqueous solutions of ammonium chloride, sodium bicarbonate, and brine. The resulting organic liquid is dried over sodium sulfate and decanted into a round bottom flask, concentrated, and the resulting residue is purified by MPLC (0 to 100% EtOAc/hexanes), then taken up in DCM and 4 N HCl in dioxane. After 1 h, the solution is concentrated under reduced pressure to give the HCl salt (compound 511-2).

Compound 511-2 is then taken up in AcOH and charged to a 30 mL reaction vial. (E)-N-((dimethylamino)methylene)benzamide (471-2, see Example 471, 2 eq) is added, and the reaction vial is sealed under a Teflon septum. The mixture is heated to 110° C. for 2 h. After cooling to 23° C., the reaction mixture is brought to pH 8 by slow addition of an aqueous solution of 4N K₂CO₃. The resulting mixture is extracted with EtOAc and rinsed sequentially with saturated, aqueous solutions of ammonium chloride, sodium bicarbonate, and brine. The resulting organic liquid is dried over sodium sulfate and decanted into a round bottom flask, concentrated, and the resulting residue is purified by HPLC (30-60% MeCN, 18 mL/min, 210 nM, 0.1% TFA. Stationary Phase: Phenomenex Luna C18, 2×25 cm) to give the title compound.

Additional compounds are prepared similarly to this method, optionally replacing Intermediate G with a suitable Intermediate in the first step and/or replacting
(E)-N-((dimethylamino)methylene)benzamide with a suitable reactant in the last step. The following compounds are prepared:
(R)-4-ethyl-5-isopropyl-1-methyl-7-(5-phenyl-1H-1,2,4-triazol-1-yl)-4,5-dihydro-[1,2,4]triazolo[4,3-f]pteridine (Example 512),
(R)-4-ethyl-5-isopropyl-7-(5-phenyl-1H-pyrazol-1-yl)-4,5-dihydro-[1,2,4]triazolo[4,3-f]pteridine (Example 513),
(R)-4-ethyl-5-isopropyl-1-methyl-7-(5-phenyl-1H-pyrazol-1-yl)-4,5-dihydro-[1,2,4]triazolo[4,3-f]pteridine (Example 514),
(S)-12a-ethyl-7-(5-phenyl-1H-1,2,4-triazol-1-yl)-10,11,12,12a-tetrahydropyrrolo[2,1-h][1,2,4]triazolo[4,3-f]pteridine (Example 515),
(S)-12a-ethyl-3-methyl-7-(5-phenyl-1H-1,2,4-triazol-1-yl)-10,11,12,12a-tetrahydropyrrolo[2,1-h][1,2,4]triazolo[4,3-f]pteridine (Example 516),
(S)-12a-ethyl-7-(5-phenyl-1H-pyrazol-1-yl)-10,11,12,12a-tetrahydropyrrolo[2,1-h][1,2,4]triazolo[4,3-f]pteridine (Example 517),
(S)-12a-ethyl-3-methyl-7-(5-phenyl-1H-pyrazol-1-yl)-10,11,12,12a-tetrahydropyrrolo[2,1-h][1,2,4]triazolo[4,3-f]pteridine (Example 518),
(R)-5-cyclobutyl-4-ethyl-7-(5-phenyl-1H-pyrazol-1-yl)-4,5-dihydro-[1,2,4]triazolo[4,3-f]pteridine (Example 519),
(R)-5-cyclobutyl-4-ethyl-1-methyl-7-(5-phenyl-1H-pyrazol-1-yl)-4,5-dihydro-[1,2,4]triazolo[4,3-f]pteridine (Example 520),
(R)-5-cyclobutyl-4-ethyl-7-(5-phenyl-1H-1,2,4-triazol-1-yl)-4,5-dihydro-[1,2,4]triazolo[4,3-f]pteridine (Example 521),
(R)-5-cyclobutyl-4-ethyl-1-methyl-7-(5-phenyl-1H-1,2,4-triazol-1-yl)-4,5-dihydro-[1,2,4]triazolo[4,3-f]pteridine (Example 522),
14a-ethyl-7-(5-phenyl-1H-pyrazol-1-yl)-10,11,12,13,14,14a-hexahydroazepino[2,1-h][1,2,4]triazolo[4,3-f]pteridine (Example 523),
14a-ethyl-3-methyl-7-(5-phenyl-1H-pyrazol-1-yl)-10,11,12,13,14,14a-hexahydroazepino[2,1-h][1,2,4]triazolo[4,3-f]pteridine (Example 524),
(R)-5-cyclobutyl-4-ethyl-7-(5-(quinolin-5-yl)-1H-1,2,4-triazol-1-yl)-4,5-dihydro-[1,2,4]triazolo[4,3-f]pteridine (Example 525),
(R)-5-cyclobutyl-4-ethyl-1-methyl-7-(5-(quinolin-5-yl)-1H-1,2,4-triazol-1-yl)-4,5-dihydro-[1,2,4]triazolo[4,3-f]pteridine (Example 526),
14a-ethyl-7-(5-phenyl-1H-1,2,4-triazol-1-yl)-10,11,12,13,14,14a-hexahydroazepino[2,1-h][1,2,4]triazolo[4,3-f]pteridine (Example 527),
14a-ethyl-3-methyl-7-(5-phenyl-1H-1,2,4-triazol-1-yl)-10,11,12,13,14,14a-hexahydroazepino[2,1-h][1,2,4]triazolo[4,3-f]pteridine (Example 528),
14a-ethyl-7-(5-(quinolin-5-yl)-1H-1,2,4-triazol-1-yl)-10,11,12,13,14,14a-hexahydroazepino[2,1-h][1,2,4]triazolo[4,3-f]pteridine (Example 529),
14a-ethyl-3-methyl-7-(5-(quinolin-5-yl)-1H-1,2,4-triazol-1-yl)-10,11,12,13,14,14a-hexahydroazepino[2,1-h][1,2,4]triazolo[4,3-f]pteridine (Example 530),
(S)-12a-ethyl-7-(5-(quinolin-5-yl)-1H-1,2,4-triazol-1-yl)-10,11,12,12a-tetrahydropyrrolo[2,1-h][1,2,4]triazolo[4,3-f]pteridine (Example 531),
(S)-12a-ethyl-3-methyl-7-(5-(quinolin-5-yl)-1H-1,2,4-triazol-1-yl)-10,11,12,12a-tetrahydropyrrolo[2,1-h][1,2,4]triazolo[4,3-f]pteridine (Example 532),
(S)-12a-ethyl-7-(5-(phenylethynyl)-1H-1,2,4-triazol-1-yl)-10,11,12,12a-tetrahydropyrrolo[2,1-h][1,2,4]triazolo[4,3-f]pteridine (Example 533), and
(S)-12a-ethyl-3-methyl-7-(5-(phenylethynyl)-1H-1,2,4-triazol-1-yl)-10,11,12,12a-tetrahydropyrrolo[2,1-h][1,2,4]triazolo[4,3-f]pteridine (Example 534).
   The following table provides the example number (column 1), Intermediate (column 2), and last step reactant (column 3) used to give the compound shown in column 4.

| Ex. | | | |
|---|---|---|---|
| No. | Int. | Last step reactant | Compound structure |
| 512 | H | 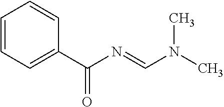 | 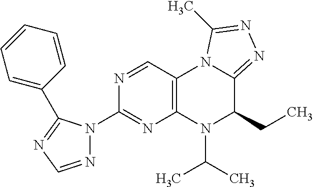 |
| 513 | G | 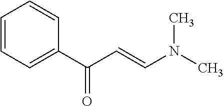 | 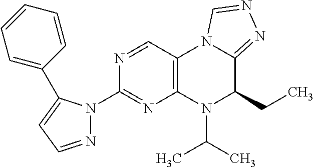 |
| 514 | H | | 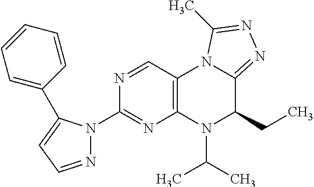 |
| 515 | XX | 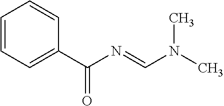 | 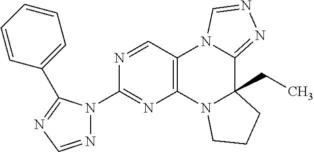 |
| 516 | XX′ | | 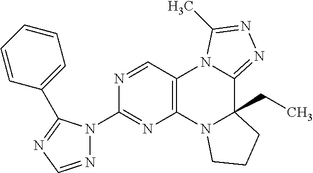 |
| 517 | XX | 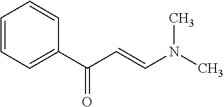 | 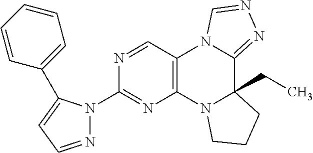 |
| 518 | XX′ | | 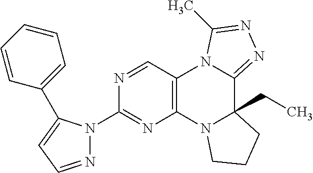 |
| 519 | C | 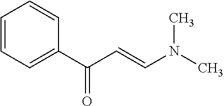 | 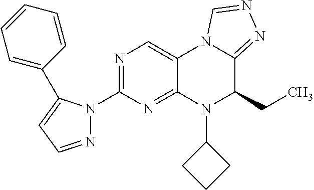 |
| 520 | D | | 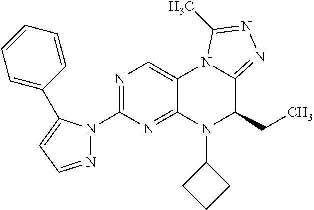 |
| 521 | C | 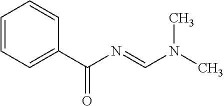 | 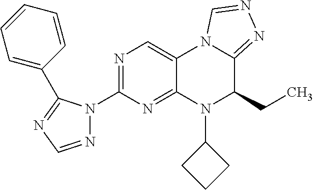 |
| 522 | D | | 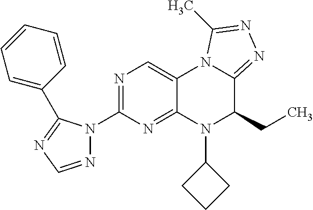 |
| 523 | X | 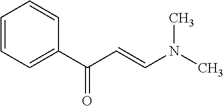 | 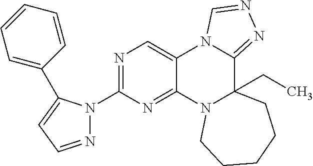 |
| 524 | X′ | | 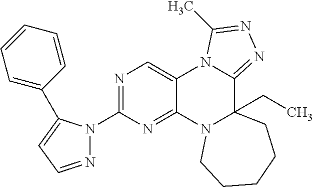 |
| 525 | C | 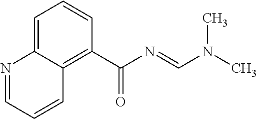 | 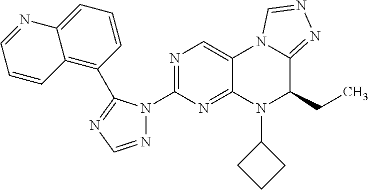 |
| 526 | D | | 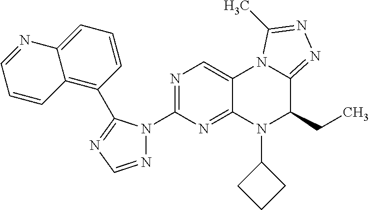 |
| 527 | X | 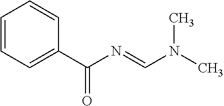 | 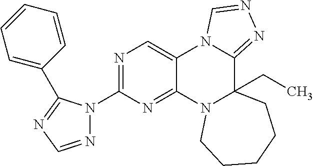 |
| 528 | X′ | | 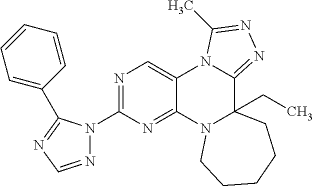 |
| 529 | X | 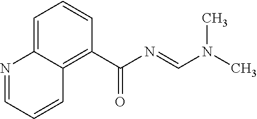 | 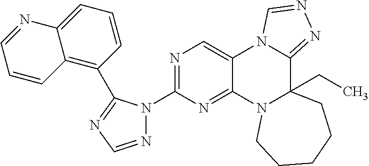 |
| 530 | X′ | | 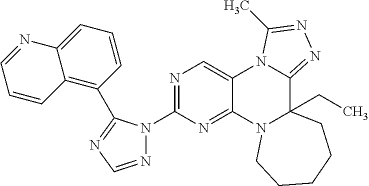 |
| 531 | XX | 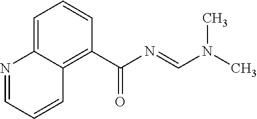 | 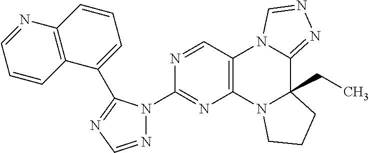 |
| 532 | XX′ | | 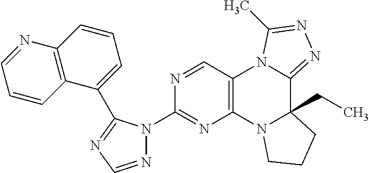 |
| 533 | XX | 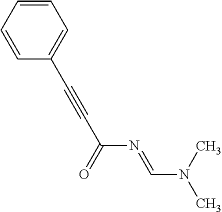 | 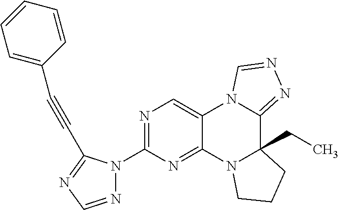 |
| 534 | XX′ | | 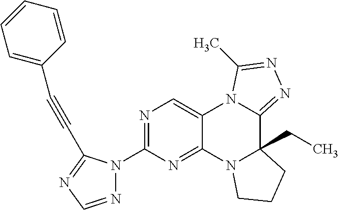 |

### Example 535

### Synthesis of (R)-5-cyclopentyl-4-ethyl-7-(4-methylpiperazin-1-yl)-4,5-dihydro-[1,2,4]triazolo[4,3-f]pteridine

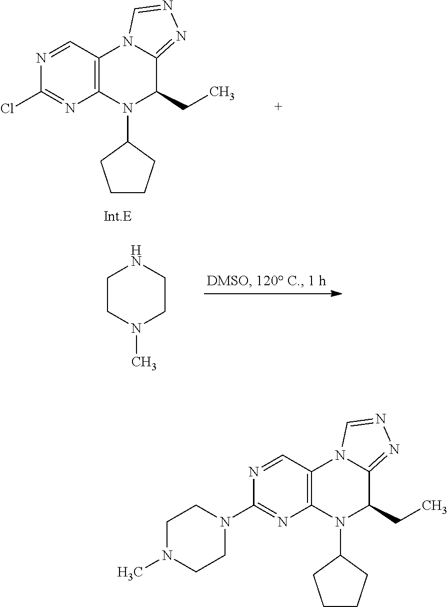

Intermediate E (0.39 mmol) and N-methylpiperazine (6 eq) in 1 mL of DMSO is heated at 120° C. in a microwave for 2 h. The reaction is diluted with water and extracted with EtOAc. The organic extracts are washed 5× with water, then dried with MgSO₄ and evaporated. The residue is purified by reverse-phase HPLC (eluting with 10-30% acetonitrile in water with 0.1% TFA over 20 min; Phenomenex Luna C-18 column, 25×2 cm) to give the title compound after lyophylization.

Additional compounds are prepared similarly to this method, optionally replacing Intermediate E with a suitable Intermediate and/or replacting N-methylpiperazine with a suitable ring reactant. The following compounds are prepared:
(R)-5-cyclopentyl-4-ethyl-1-methyl-7-(4-methylpiperazin-1-yl)-4,5-dihydro-[1,2,4]triazolo[4,3-f]pteridine (Example 536),
(R)-4-(5-cyclopentyl-4-ethyl-4,5-dihydro-[1,2,4]triazolo[4,3-f]pteridin-7-yl)piperazin-2-one (Example 537),
(R)-4-(5-cyclopentyl-4-ethyl-1-methyl-4,5-dihydro-[1,2,4]triazolo[4,3-f]pteridin-7-yl)piperazin-2-one (Example 538),
(R)-5-cyclopentyl-4-ethyl-7-(4-(pyrazin-2-yl)piperazin-1-yl)-4,5-dihydro-[1,2,4]triazolo[4,3-f]pteridine (Example 539), and
(R)-5-cyclopentyl-4-ethyl-1-methyl-7-(4-(pyrazin-2-yl)piperazin-1-yl)-4,5-dihydro-[1,2,4]triazolo[4,3-f]pteridine (Example 540).
   The following table provides the example number (column 1), Intermediate (column 2), and ring reactant (column 3) used to give the compound shown in column 4.

| Ex. No. | Int. | Ring reactant | Compound structure |
|---|---|---|---|
| 536 | F | 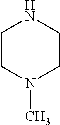 | 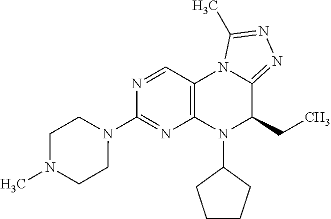 |
| 537 | E | 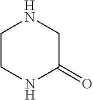 | 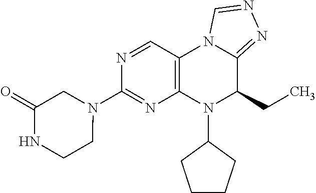 |
| 538 | F | | 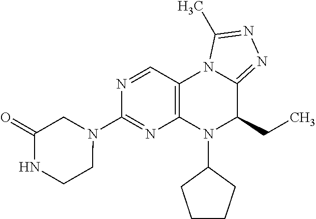 |
| 539 | E | 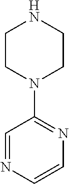 | 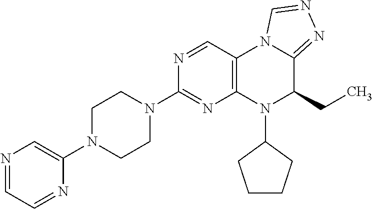 |
| 540 | F | | 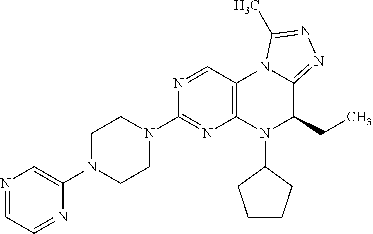 |

### Example 541

### Synthesis of (4R)-S-(3,3-difluorocyclopentyl)-7-(2-(3,4-difluorophenyl)-1H-imidazol-1-yl)-4-ethyl-4,5-dihydro-[1,2,4]triazolo[4,3-f]pteridine

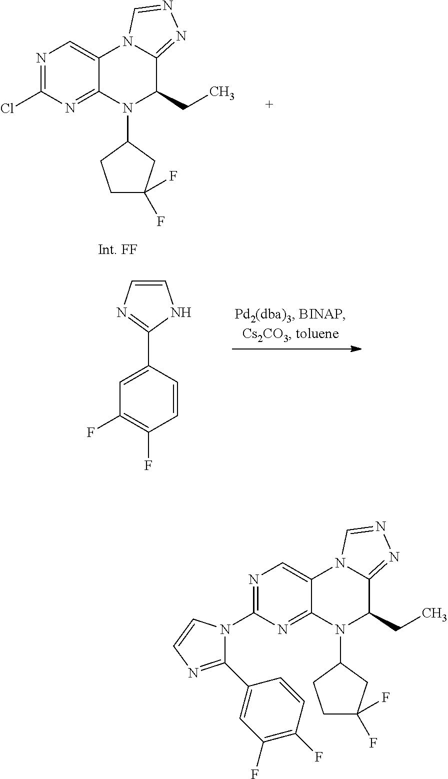

To a stirring mixture of Intermediate xx (1 eq) in 1.0 mL of toluene, Pd₂(dba)₃ (0.4 eq), BINAP (0.8 eq), 2-(3,4-difluorophenyl)-1H-imidazole (1.2 eq), and Cs₂CO₃ (3 eq) are added. The reaction mixture is heated under microwave condition at 140° C. for 1 h. The crude product mixture is purified by MPLC and further purified by preparative HPLC to give the title compound.

Additional compounds are prepared similarly to this method, optionally replacing Intermediate FF with a suitable Intermediate and/or replacting 2(3,4-difluorophenyl)-1H-imidazole with a suitable ring reactant. In some instances, where a racemic mixture results, the two enantiomers may be isolated by chiral chromatography.

The following compounds are prepared:
(4R)-S-(3,3-difluorocyclopentyl)-7-(2-(3,4-difluorophenyl)-1H-imidazol-1-yl)-4-ethyl-1-methyl-4,5-dihydro-[1,2,4]triazolo[4,3-f]pteridine (Example 542),
(4R)-S-(3,3-difluorocyclopentyl)-4-ethyl-7-(2-(4-fluorophenyl)-1H-imidazol-1-yl)-4,5-dihydro-[1,2,4]triazolo[4,3-f]pteridine (Example 543),
(4R)-S-(3,3-difluorocyclopentyl)-4-ethyl-7-(2-(4-fluorophenyl)-1H-imidazol-1-yl)-1-methyl-4,5-dihydro-[1,2,4]triazolo[4,3-f]pteridine (Example 544),
(R)-5-(3,3-difluorocyclobutyl)-4-ethyl-7-(2-(4-fluorophenyl)-1H-imidazol-1-yl)-4,5-dihydro-[1,2,4]triazolo[4,3-f]pteridine (Example 545),
(R)-5-(3,3-difluorocyclobutyl)-4-ethyl-7-(2-(4-fluorophenyl)-1H-imidazol-1-yl)-1-methyl-4,5-dihydro-[1,2,4]triazolo[4,3-f]pteridine (Example 546),
(R)-5-(3,3-difluorocyclobutyl)-7-(2-(2,4-difluorophenyl)-1H-imidazol-1-yl)-4-ethyl-4,5-dihydro-[1,2,4]triazolo[4,3-f]pteridine (Example 547),
(R)-5-(3,3-difluorocyclobutyl)-7-(2-(2,4-difluorophenyl)-1H-imidazol-1-yl)-4-ethyl-1-methyl-4,5-dihydro-[1,2,4]triazolo[4,3-f]pteridine (Example 548),
5-isopropyl-7-(2-phenyl-1H-imidazol-1-yl)-5H-spiro[[1,2,4]triazolo[4,3-f]pteridine-4,1′-cyclobutane] (Example 549),
5-isopropyl-1-methyl-7-(2-phenyl-1H-imidazol-1-yl)-5H-spiro[[1,2,4]triazolo[4,3-f]pteridine-4,1′-cyclobutane] (Example 550),
7-(2-(4-fluorophenyl)-1H-imidazol-1-yl)-5-isopropyl-5H-spiro[[1,2,4]triazolo[4,3-f]pteridine-4,1′-cyclobutane] (Example 551),
7-(2-(4-fluorophenyl)-1H-imidazol-1-yl)-5-isopropyl-1-methyl-5H-spiro[[1,2,4]triazolo[4,3-f]pteridine-4,1′-cyclobutane] (Example 552),
4-ethyl-7-(2-(4-fluorophenyl)-1H-imidazol-1-yl)-5-phenyl-4,5-dihydro-[1,2,4]triazolo[4,3-f]pteridine (Example 553),
4-ethyl-7-(2-(4-fluorophenyl)-1H-imidazol-1-yl)-1-methyl-5-phenyl-4,5-dihydro-[1,2,4]triazolo[4,3-f]pteridine (Example 554),
(R)-7-(2-(2,4-difluorophenyl)-1H-imidazol-1-yl)-4-(2,2,2-trifluoroethyl)-5-(3,3,3-trifluoropropyl)-4,5-dihydro-[1,2,4]triazolo[4,3-f]pteridine and (S)-7-(2-(2,4-difluorophenyl)-1H-imidazol-1-yl)-4-(2,2,2-trifluoroethyl)-5-(3,3,3-trifluoropropyl)-4,5-dihydro-[1,2,4]triazolo[4,3-f]pteridine (Example 555),
(R)-7-(2-(2,4-difluorophenyl)-1H-imidazol-1-yl)-1-methyl-4-(2,2,2-trifluoroethyl)-5-(3,3,3-trifluoropropyl)-4,5-dihydro-[1,2,4]triazolo[4,3-f]pteridine and (S)-7-(2-(2,4-difluorophenyl)-1H-imidazol-1-yl)-1-methyl-4-(2,2,2-trifluoroethyl)-5-(3,3,3-trifluoropropyl)-4,5-dihydro-[1,2,4]triazolo[4,3-f]pteridine (Example 556),
7-(2-(4-fluorophenyl)-1H-imidazol-1-yl)-5-isopropyl-4,4-dimethyl-4,5-dihydro-[1,2,4]triazolo[4,3-f]pteridine (Example 557),
7-(2-(4-fluorophenyl)-1H-imidazol-1-yl)-5-isopropyl-1,4,4-trimethyl-4,5-dihydro-[1,2,4]triazolo[4,3-f]pteridine (Example 558),
(R)-5-(cyclopropylmethyl)-4-ethyl-7-(2-(4-fluorophenyl)-1H-imidazol-1-yl)-4,5-dihydro-[1,2,4]triazolo[4,3-f]pteridine (Example 559),
(R)-5-(cyclopropylmethyl)-4-ethyl-7-(2-(4-fluorophenyl)-1H-imidazol-1-yl)-1-methyl-4,5-dihydro-[1,2,4]triazolo[4,3-f]pteridine (Example 560), 4-ethyl-5-(4-fluorophenyl)-7-(2-(4-fluorophenyl)-1H-imidazol-1-yl)-4,5-dihydro-[1,2,4]triazolo[4,3-f]pteridine (Example 561),
4-ethyl-5-(4-fluorophenyl)-7-(2-(4-fluorophenyl)-1H-imidazol-1-yl)-1-methyl-4,5-dihydro-[1,2,4]triazolo[4,3-f]pteridine (Example 562),
(R)-3-(4-ethyl-7-(2-(4-fluorophenyl)-1H-imidazol-1-yl)-[1,2,4]triazolo[4,3-f]pteridin-5(4H)-yl)benzonitrile and (S)-3-(4-ethyl-7-(2-(4-fluorophenyl)-1H-imidazol-1-yl)-[1,2,4]triazolo[4,3-f]pteridin-5(4H)-yl)benzonitrile (Example 563),
(R)-3-(4-ethyl-7-(2-(4-fluorophenyl)-1H-imidazol-1-yl)-1-methyl-[1,2,4]triazolo[4,3-f]pteridin-5(4H)-yl)benzonitrile and (S)-3-(4-ethyl-7-(2-(4-fluorophenyl)-1H-imidazol-1-yl)-1-methyl-[1,2,4]triazolo[4,3-f]pteridin-5(4H)-yl)benzonitrile (Example 564),
4-ethyl-7-(2-(4-fluorophenyl)-1H-imidazol-1-yl)-5-(1-((2-(trimethylsilyl)ethoxy)methyl)-1H-pyrazol-4-yl)-4,5-dihydro-[1,2,4]triazolo[4,3-f]pteridine (Example 565),
4-ethyl-7-(2-(4-fluorophenyl)-1H-imidazol-1-yl)-1-methyl-5-(1-((2-(trimethylsilyl)ethoxy)methyl)-1H-pyrazol-4-yl)-4,5-dihydro-[1,2,4]triazolo[4,3-f]pteridine (Example 566),
4-ethyl-7-(2-(4-fluorophenyl)-1H-imidazol-1-yl)-5-(3-(pyrimidin-5-yl)phenyl)-4,5-dihydro-[1,2,4]triazolo[4,3-f]pteridine (Example 567),
4-ethyl-7-(2-(4-fluorophenyl)-1H-imidazol-1-yl)-1-methyl-5-(3-(pyrimidin-5-yl)phenyl)-4,5-dihydro-[1,2,4]triazolo[4,3-f]pteridine (Example 568),
(4R)-S-(1-cyclopropylethyl)-4-ethyl-7-(2-phenyl-1H-imidazol-1-yl)-4,5-dihydro-[1,2,4]triazolo[4,3-f]pteridine (Example 569),
(4R)-S-(1-cyclopropylethyl)-4-ethyl-1-methyl-7-(2-phenyl-1H-imidazol-1-yl)-4,5-dihydro-[1,2,4]triazolo[4,3-f]pteridine (Example 570),
(4R)-S-(1-cyclopropylethyl)-4-ethyl-7-(2-(4-fluorophenyl)-1H-imidazol-1-yl)-4,5-dihydro-[1,2,4]triazolo[4,3-f]pteridine (Example 571),
(4R)-S-(1-cyclopropylethyl)-4-ethyl-7-(2-(4-fluorophenyl)-1H-imidazol-1-yl)-1-methyl-4,5-dihydro-[1,2,4]triazolo[4,3-f]pteridine (Example 572),
4-(4-ethyl-7-(2-(4-fluorophenyl)-1H-imidazol-1-yl)-[1,2,4]triazolo[4,3-f]pteridin-5(4H)-yl)benzonitrile (Example 573),
4-(4-ethyl-7-(2-(4-fluorophenyl)-1H-imidazol-1-yl)-1-methyl-[1,2,4]triazolo[4,3-f]pteridin-5(4H)-yl)benzonitrile (Example 574),
5-(4-chlorophenyl)-4-ethyl-7-(2-(4-fluorophenyl)-1H-imidazol-1-yl)-4,5-dihydro-[1,2,4]triazolo[4,3-f]pteridine (Example 575), and
5-(4-chlorophenyl)-4-ethyl-7-(2-(4-fluorophenyl)-1H-imidazol-1-yl)-1-methyl-4,5-dihydro-[1,2,4]triazolo[4,3-f]pteridine (Example 576).
   The following table provides the example number (column 1), Intermediate (column 2), and ring reactant (column 3) used to give the compound shown in column 4.

| Ex. No. | Int. | Ring reactant | Compound structure |
|---|---|---|---|
| 542 | FF′ | 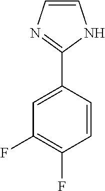 | 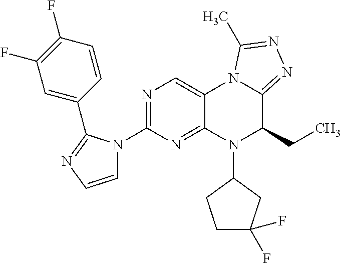 |
| 543 | FF | 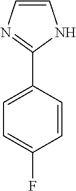 | 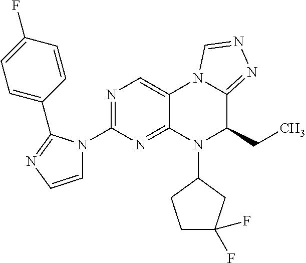 |
| 544 | FF′ | | 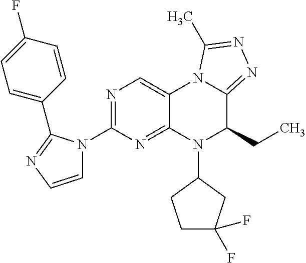 |
| 545 | V | 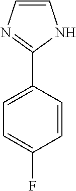 | 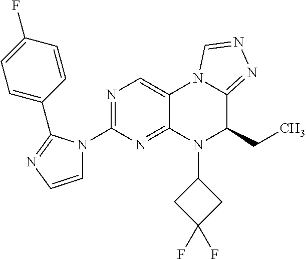 |
| 546 | V′ | | 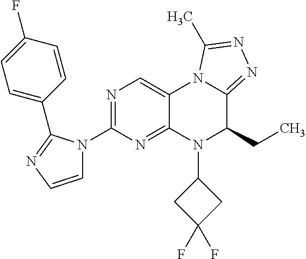 |
| 547 | V | 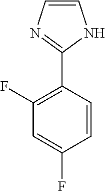 | 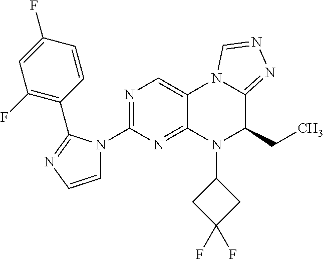 |
| 548 | V′ | | 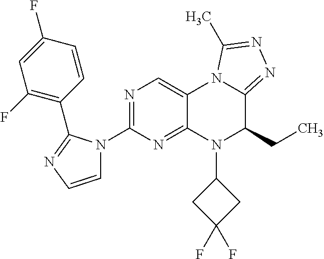 |
| 549 | GG | 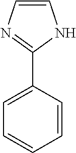 | 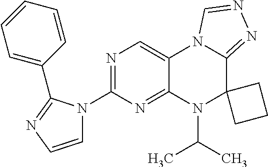 |
| 550 | GG′ | | 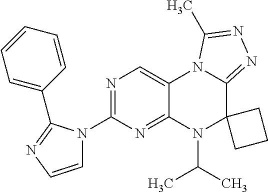 |
| 551 | GG | 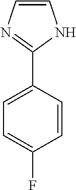 | 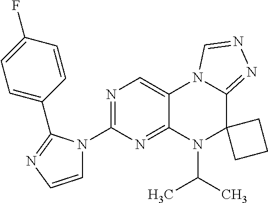 |
| 552 | GG′ | | 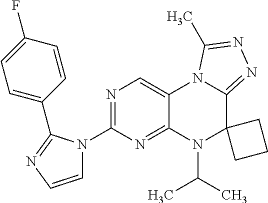 |
| 553 | CC | 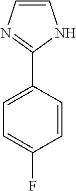 | 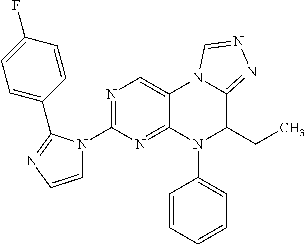 |
| 554 | CC′ | | 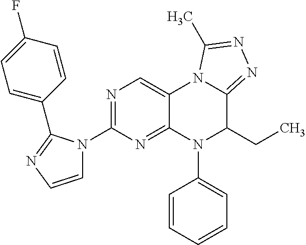 |
| 555(R) | BB | 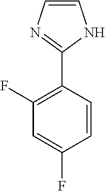 | 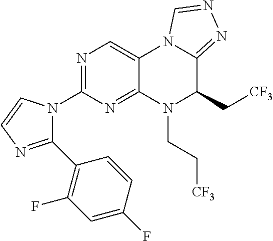 |
| 555(S) | | | 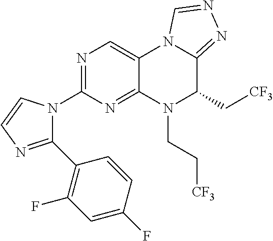 |
| 556(R) | BB′ | 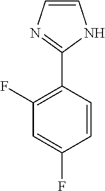 | 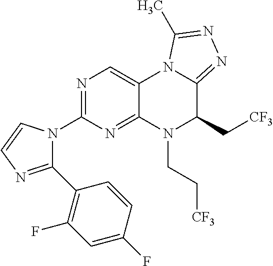 |
| 556(S) | | | 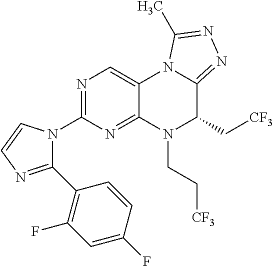 |
| 557 | HH | 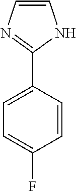 | 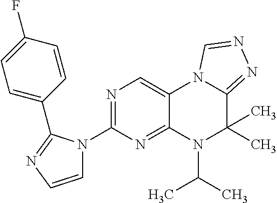 |
| 558 | HH′ | | 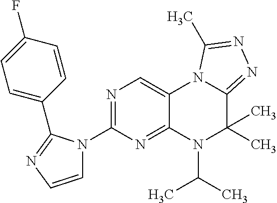 |
| 559 | DD | 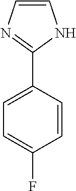 | 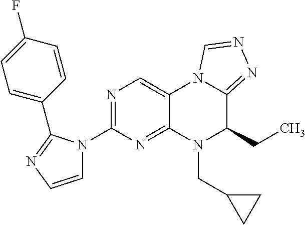 |
| 560 | DD′ | | 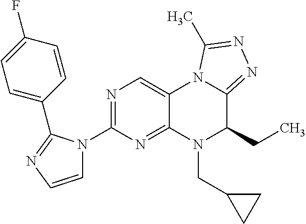 |
| 561 | EE | 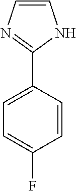 | 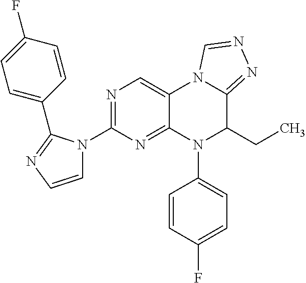 |
| 562 | EE′ | | 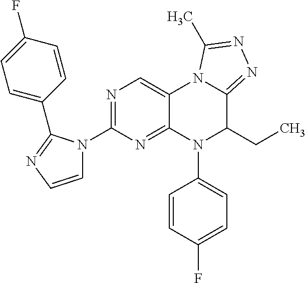 |
| 563(R) | OO | 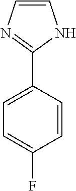 | 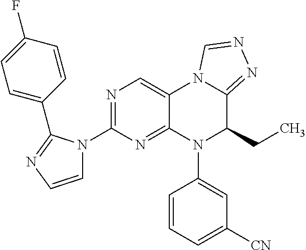 |
| 563(S) | | | 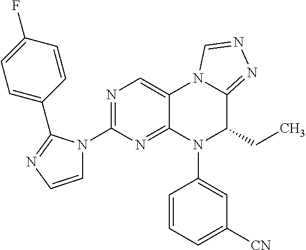 |
| 564(R) | OO′ | 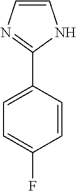 | 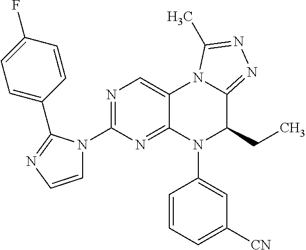 |
| 564(S) | | | 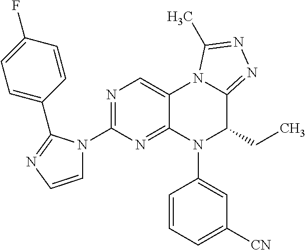 |
| 565 | KK | 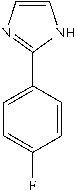 | 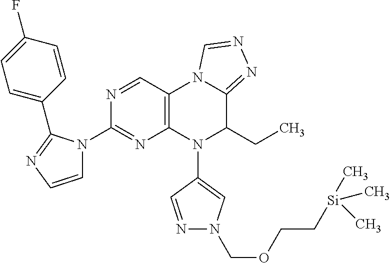 |
| 566 | KK′ | | 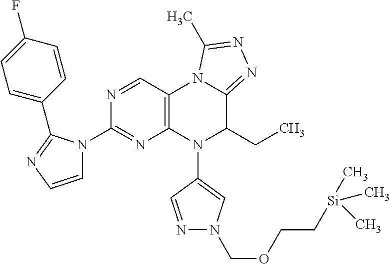 |
| 567 | MM | 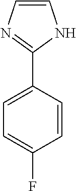 | 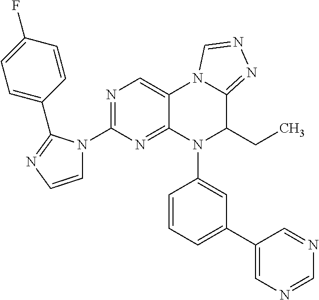 |
| 568 | MM′ | | 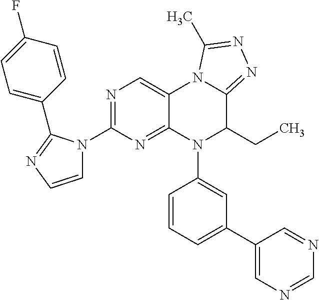 |
| 569 | SS | 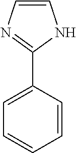 | 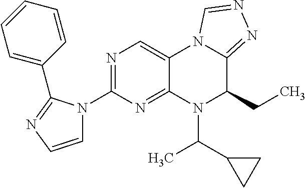 |
| 570 | SS′ | | 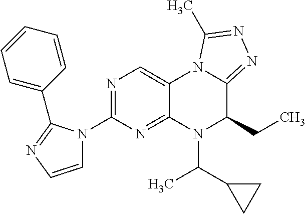 |
| 571 | SS | 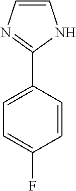 | 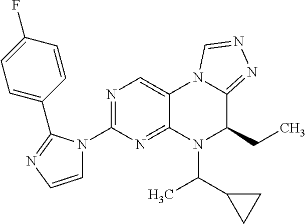 |
| 572 | SS′ | | 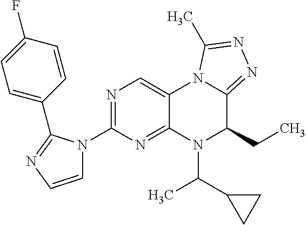 |
| 573 | PP | 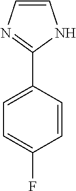 | 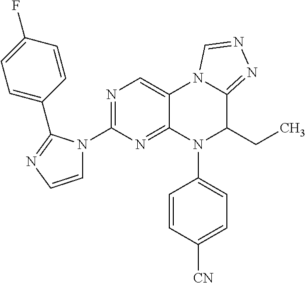 |
| 574 | PP′ | | 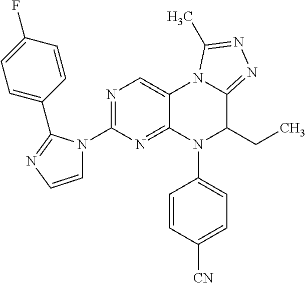 |
| 575 | TT | 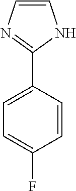 | 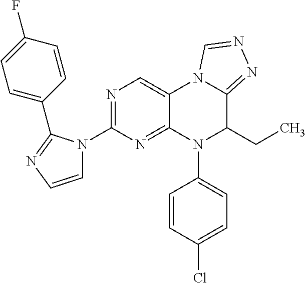 |
| 576 | TT′ | | 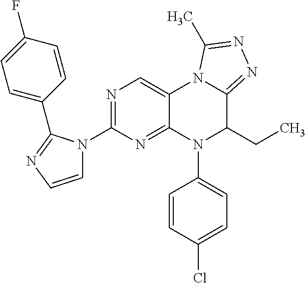 |

### Example 577 and Example 578

### Synthesis of (R)-5-cyclopentyl-4-ethyl-7-(4-phenyl-1H-pyrazol-3-yl)-4,5-dihydro-[1,2,4]triazolo[4,3-f]pteridine (577) and (R)-5-cyclopentyl-4-ethyl-1-methyl-7-(4-phenyl-1H-pyrazol-3-yl)-4,5-dihydro-[1,2,4]triazolo[4,3-f]pteridine (578)

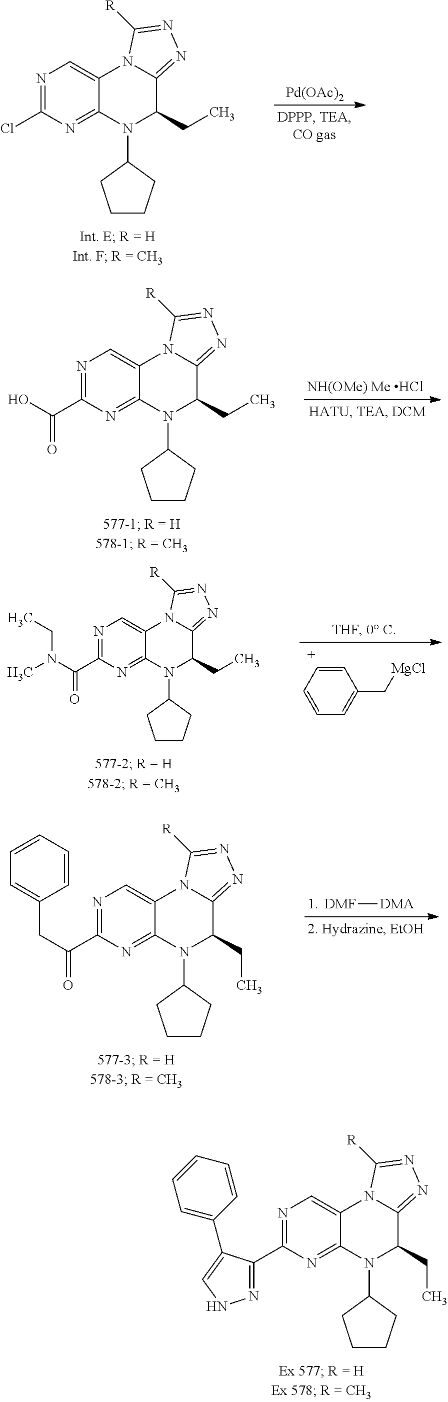

Intermediate E or Intermediat F (34.01 mmol) is dissolved in 7.5% DMSO in tBuOH and Pd(OAc)₂ (5.1 mmol), DPPP (5.1 mmol) and TEA (76.5 mmol) are added. The solution is stirred at 80° C. for 10 h under CO (10 atm). The solvent is removed under reduced pressure and the residue is dissolved in EtOAc. The organic layer is washed with water and brine, dried with Na₂SO₄, and purified by silica gel column (DCM:MeOH=20:1) to give compound 577-1 or 578-1.

Compound 577-1 or 578-1 (6.58 mmol) is dissolved in DCM, NH(OMe)Me.HCl (7.90 mmol) is added, followed by the addition of TEA (19.74 mmol) and HATU (7.90 mmol) to the solution at 0° C. The mixture is warmed to rt and stirred for 2 h, then washed with water, brine, dried and the solvent removed. The resulting material is purified by silica gel column (PE:EtOAc:MeOH=1:1:0.1) to give compound 577-2 or 578-2.

Compound 577-2 or 578-2 (4.32 mmol) is dissolved in dry THF and cooled to 0° C. Benzyl magnesium chloride (2M in THF, 5.19 mmol) is added drop-wise. The mixture is stirred for 2 h at 0° C., then the reaction quenched with water at 0° C. The THF is removed and the water layer is extracted with EtOAc. The organic layer is washed with brine, dried and purified by silica gel column (PE:EtOAc=2:1) to give compound 577-3 or 578-3.

Compound 577-3 or 578-3 (0.53 mmol) is dissolved in DMF-DMA. The mixture is refluxed for 2 h and the solvent is removed. The resulting oil is dissolved in DMF and excess hydrazine hydrogen chloride is added and this mixture is stirred at 110° C. for 18 h. The mixture is washed with water, extracted with EtOAc, the organic layer is dried, evaporated and purified by silica gel column (PE:EtOAc:MeOH=1:1:0.2) to give the title compounds.

### Example 579 and Example 580

### Synthesis of (R)-4-ethyl-5-isopropyl-7-(2-phenyl-4,5-dihydro-1H-imidazol-1-yl)-4,5-dihydro-[1,2,4]triazolo[4,3-f]pteridine (579) and (R)-4-ethyl-5-isopropyl-1-methyl-7-(2-phenyl-4,5-dihydro-1H-imidazol-1-yl)-4,5-dihydro-[1,2,4]triazolo[4,3-f]pteridine (580)

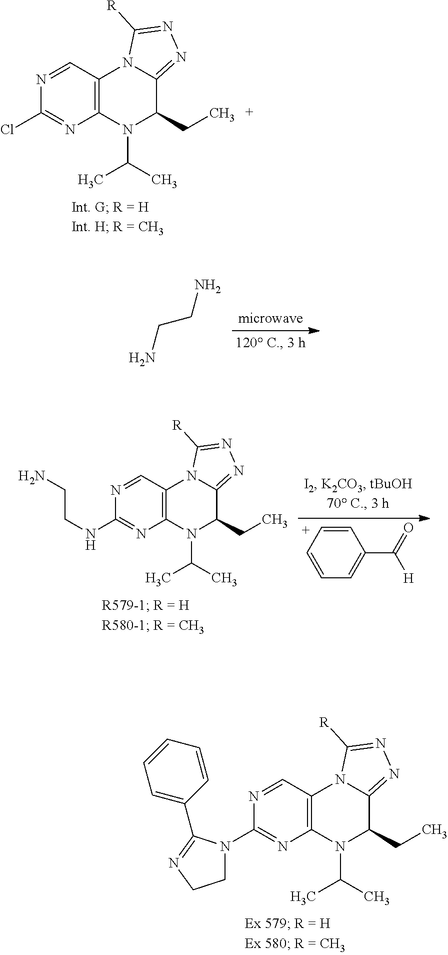

Intermediate G or Intermediate H (1 mmol) in ethylenediamine (10 mmol) is heated at 120° C. in a microwave for 3 h. The reaction is evaporated, taken up in EtOAc and washed 3× with water, then dried with MgSO₄ and evaporated to give compound 579-1 or 580-1.

Compound 579-1 or 580-1 (1.1 mmol) and benzaldehyde (1.1 mmol) are stirred in tBuOH at rt for 18 h, then K₂CO₃ (solid, 3 mmol) and 1₂ (1.25 mmol) are added. The mixture is stirred at 70° C. for 3 h, then filtered, evaporated and partitioned between CHCl₃ and water. The organic layer is washed with aqueous saturated NaHCO₃ solution and brine, then dried with MgSO₄ and evaporated. The residue is purified by HPLC (first: reverse phase, then further purified with normal phase using a ChiralPak AD column 2×25 cm, 5 micron packing) to give the title compound.

(R)-7-(2-cyclopropyl-4,5-dihydro-1H-imidazol-1-yl)-4-ethyl-5-isopropyl-1-methyl-4,5-dihydro-[1,2,4]triazolo[4,3-f]pteridine (Example 581) and (R)-7-(2-cyclopropyl-4,5-dihydro-1H-imidazol-1-yl)-4-ethyl-5-isopropyl-1-methyl-4,5-dihydro-[1,2,4]triazolo[4,3-f]pteridine (Example 582)

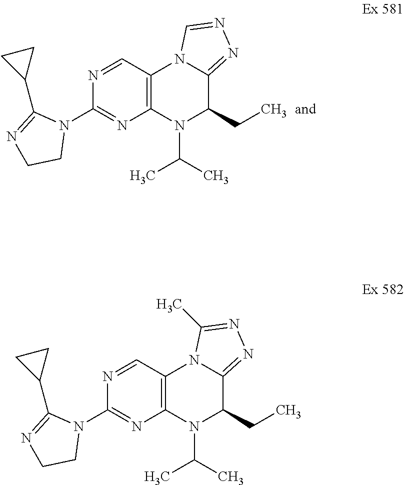

are prepared similarly, with cyclopropanecarbaldehyde instead of benzaldehyde in the last step.

### Example 583 and Example 584

### Synthesis of (5)-12a-ethyl-7-(2-phenyl-1H-imidazol-1-yl)-12,12a-dihydropyrrolo[2,1-h][1,2,4]triazolo[4,3-f]pteridin-10(11H)-one (583) and (5)-12a-ethyl-3-methyl-7-(2-phenyl-1H-imidazol-1-yl)-12,12a-dihydropyrrolo[2,1-h][1,2,4]triazolo[4,3-f]pteridin-10(11H)-one (584)

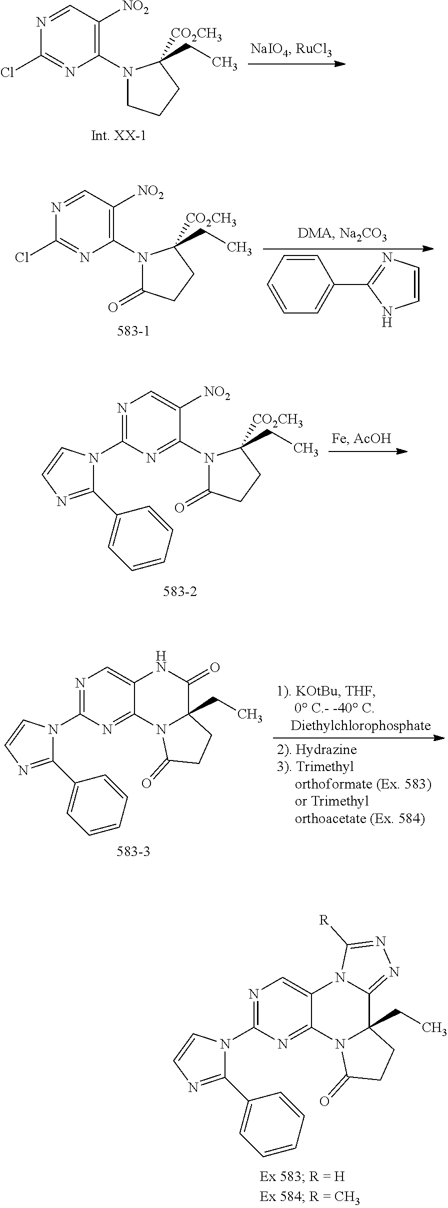

Intermediate XX-1 (1.657 mmol, 0.521 g) in 10 mL of CH₃CN was added to a solution of sodium periodate (8.285 mmol, 1.77 g) and ruthenium(III) chloride hydrate (0.165 mmol, 0.034 g) in 10 mL of H₂O. The reaction mixture was stirred at rt for 72 h, then diluted with 20 mL of isopropanol and stirred for 1 h, then concentrated. The resulting residue was dissolved in 25 mL of EtOAc and washed with 10 mL of water. The organic layer was dried with Na₂SO₄, filtered and concentrated. The resulting residue was purified by flash chromatography (30% EtOAc in hexanes) to give (S)-methyl 1-(2-chloro-5-nitropyrimidin-4-yl)-2-ethyl-5-oxopyrrolidine-2-carboxylate (compound 583-1).

The resulting residue (compound 583-1) was dissolved in 2 mL of DMA and 2-phenyl-1H-imidazole (0.176 mmol, 0.025 g) and sodium carbonate (0.176 mmol, 0.018 g) were added. The reaction mixture was microwaved for 1 h at 150° C., then diluted with 20 mL of EtOAc and washed with 10 mL of H₂O. The organic layer was dried with Na₂SO₄, filtered and concentrated. The resulting residue was purified by flash chromatography (70% EtOAc in hexanes) to give (S)-methyl 1-(2-chloro-5-nitropyrimidin-4-yl)-2-ethyl-5-oxopyrrolidine-2-carboxylate (compound 583-2).

The resulting residue (compound 583-2) was dissolved in 3 mL of AcOH and iron (0.446 mmol, 0.024 g) was added. The reaction mixture was fitted with a reflux condenser, was plunged into a preheated 90° C. oil bath, and was stirred for 25 minutes. The reaction mixture was cooled to rt, diluted with 15 mL fo EtOAc, washed with 5 mL of H₂O, 5 mL of aqueous saturated NaHCO₃, dried with Na₂SO₄, filtered and concentrated to give (S)-6a-ethyl-2-(2-phenyl-1H-imidazol-1-yl)-7,8-dihydropyrrolo[2,1-h]pteridine-6,9(5H,6aH)-dione (compound 583-3).

A solution of compound 583-3 in THF is stirred at −20° C. and potassium tert-butoxide (1.3 eq) is added over 5 min. The reaction mixture is warmed up to 0° C. for 25 min after complete addition. The reaction mixture is cooled to −40° C. and diethylchlorophosphate (1.4 eq) is added. The reaction mixture is warmed up to rt for 45 min. To the resulting mixture, 1M hydrazine (10 eq) is added and the reaction mixture is stirred at rt for 18 h. The reaction mixture is concentrated under reduced pressure and diluted with DCM and a saturated NaHCO₃ solution. The organic layer is dried over MgSO₄ and concentrated under pressure. The resulting material is purified via the iso column, then dissolved in trimethyl orthoformate or trimethyl orthoacetate (10 eq) and heated to 110° C. for 1 h. The reaction mixture is concentrated under reduced pressure and purified via silica gel column chromatography to give the title compounds.

### Example 585 and Example 586

### Synthesis of (S)-12a-ethyl-7-(2-(3-(pyridin-3-yl)phenyl)-1H-imidazol-1-yl)-10,11,12,12a-tetrahydropyrrolo[2,1-h][1,2,4]triazolo[4,3-f]pteridine (585) and (5)-12a-ethyl-3-methyl-7-(2-(3-(pyridin-3-yl)phenyl)-1H-imidazol-1-yl)-10,11,12,12a-tetrahydropyrrolo[2,1-h][1,2,4]triazolo[4,3-f]pteridine (586)

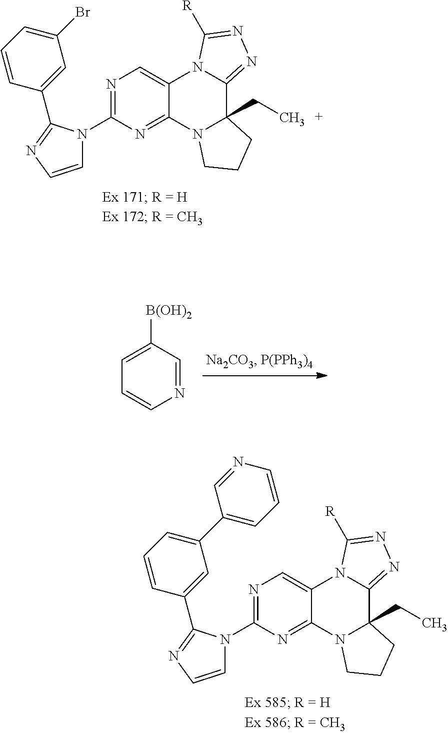

The compound of Example 171 or Example 172 (0.118 mmol) is added to a solution of 3-pyridyl boronic acid (0.593 mmol), Na₂CO₃ (0.593 mmol), and Pd(PPh₃)₄ (0.029 mmol) in 1 mL of DME and 0.5 mL of water. The reaction mixture is microwaved for 40 minutes at 135° C., then diluted with DCM, washed with water, dried with Na₂SO₄, filtered and concentrated. The resulting residue is purified by reverse phase HPLC to give the title compounds.

### Example 587 and Example 588

### Synthesis of (S)-7-(2-(3-(1H-1,2,4-triazol-1-yl)phenyl)-1H-imidazol-1-yl)-12a-ethyl-10,11,12,12a-tetrahydropyrrolo[2,1-h][1,2,4]triazolo[4,3-f]pteridine (587) and (S)-7-(2-(3-(1H-1,2,4-triazol-1-yl)phenyl)-1H-imidazol-1-yl)-12a-ethyl-3-methyl-10,11,12,12a-tetrahydropyrrolo[2,1-h][1,2,4]triazolo[4,3-f]pteridine (588)

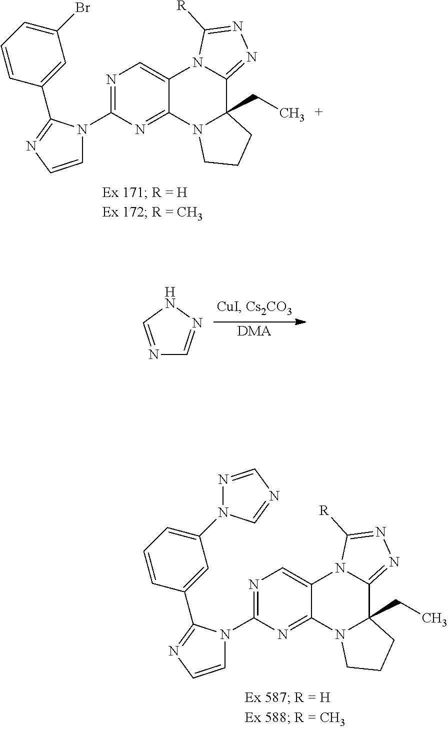

The compound of Example 171 or Example 172 (0.118 mmol) is added to a solution of 1,2,4-triazole (0.593 mmol), copper iodide (0.007 mmol), N1,N2-dimethylcyclohexane-1,2-diamine (0.023 mmol), and Cs₂CO₃ (0.593 mmol) in 1 mL of DMA. The reaction mixture is microwaved at 185° C. for 1 h. The reaction mixture is diluted with DCM, washed with water, dried with Na₂SO₄, filtered and concentrated. The resulting residue is purified by reverse phase HPLC to give the title compounds.

(S)-7-(2-(3-(1H-pyrazol-1-yl)phenyl)-1H-imidazol-1-yl)-12a-ethyl-10,11,12,12a-tetrahydropyrrolo[2,1-h][1,2,4]triazolo[4,3-f]pteridine (Example 589) and (S)-7-(2-(3-(1H-pyrazol-1-yl)phenyl)-1H-imidazol-1-yl)-12a-ethyl-3-methyl-10,11,12,12a-tetrahydropyrrolo[2,1-h][1,2,4]triazolo[4,3-f]pteridine (Example 590)

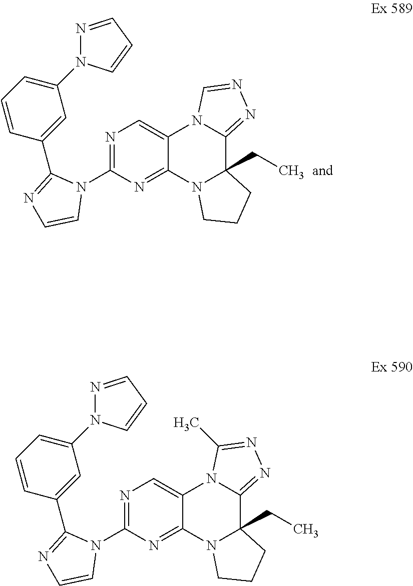

are prepared similarly, with 1H-pyrazole instead of 1H-1,2,4-triazole.

### Example 591 and Example 592

### Synthesis of tert-butyl 4-ethyl-7-(2-phenyl-1H-imidazol-1-yl)-[1,2,4]triazolo[4,3-f]pteridin-5(4H)-ylcarbamate (591) and tert-butyl 4-ethyl-1-methyl-7-(2-phenyl-1H-imidazol-1-yl)-[1,2,4]triazolo[4,3-f]pteridin-5(4H)-ylcarbamate (592)

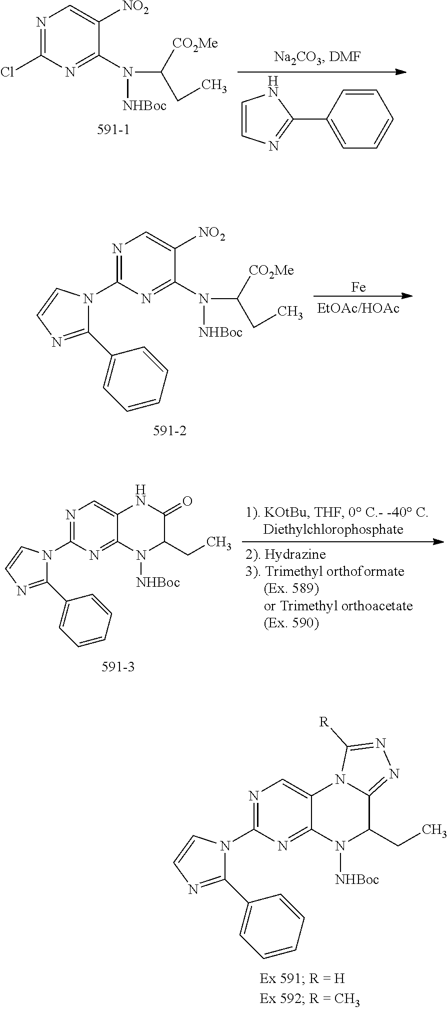

The title compounds are prepared similarly to the methods described in Example 13, with tert-butyl 2-(2-chloro-5-nitropyrimidin-4-yl)-2-(1-methoxy-1-oxobutan-2-yl)hydrazinecarboxylate (compound 591-1, prepared as described in PCT publication WO 2009130016, the contents of which are hereby incorporated by reference with respect to this compound) instead of Intermediate E-0, and 2-(4-fluorophenyl)imidazole instead of imidazole in the first step.

### Example 593 and Example 594

### Synthesis of 4-ethyl-7-(2-phenyl-1H-imidazol-1-yl)-[1,2,4]triazolo[4,3-f]pteridin-5(4H)-amine (593) and 4-ethyl-1-methyl-7-(2-phenyl-1H-imidazol-1-yl)-[1,2,4] triazolo[4,3-f]pteridin-5(4H)-amine (594)

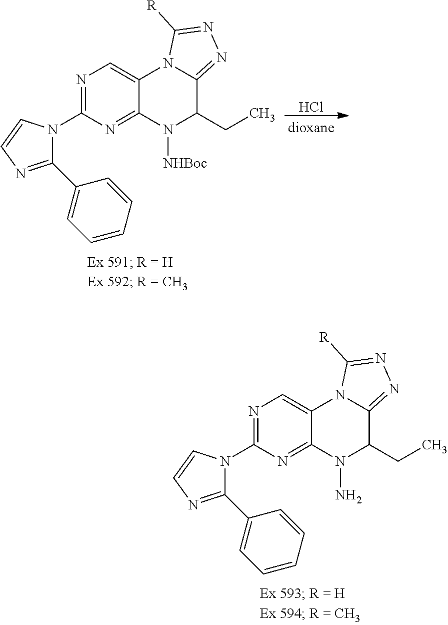

The compound of Example 591 or Example 592 (0.63 mmol) is dissolved in 4N HCl (1 mL dioxane) at 0° C., then allowed to warm to rt for 1 h. The reaction mixture is concentrated and purified by preparative HPLC to give the title compounds.

### Example 595 and Example 596

### Synthesis of 4-ethyl-7-(2-phenyl-1H-imidazol-1-yl)-5-(pyrrolidin-1-yl)-4,5-dihydro-[1,2,4]triazolo[4,3-f]pteridine (595) and 4-ethyl-1-methyl-7-(2-phenyl-1H-imidazol-1-yl)-5-(pyrrolidin-1-yl)-4,5-dihydro-[1,2,4]triazolo[4,3-f]pteridine (596)

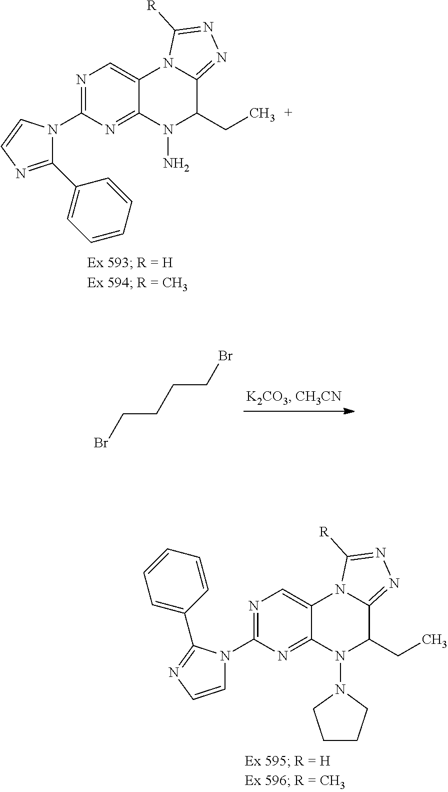

The compound of Example 593 or 594 (0.073 mmol) is combined with 1,4-dibromobutane (0.42 mmol) and potassium carbonate (0.27 mmol) in 0.2 mL of CH₃CN. This mixture is heated to 80° C. for 19 h, then filtered, washed with EtOAc, and the filtrate concentrated under reduced pressure. The residue is purified by HPLC to give the title compounds.

The compound of Example 593 or 594 can be reacted similarly, with methyl iodide instead of 1,4-dibromobutane, using DMF instead of acetonitrile as solvent, to give 1-(4-ethyl-5-(methylamino)-4,5-dihydro-[1,2,4]triazolo[4,3-f]pteridin-7-yl)-3-methyl-2-phenyl-1H-imidazol-3-ium (Example 597) and 1-(4-ethyl-1-methyl-5-(methylamino)-4,5-dihydro-[1,2,4]triazolo[4,3-f]pteridin-7-yl)-3-methyl-2-phenyl-1H-imidazol-3-ium (Example 598):

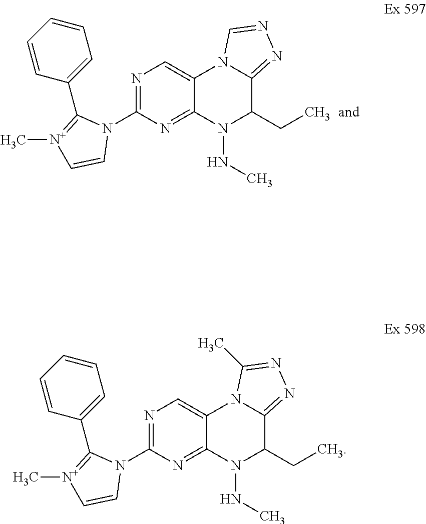

### Example 599

### Synthesis of (S)-12a-ethyl-7-(1H-imidazol-1-yl)-10,11,12,12a-tetrahydropyrrolo[2,1-h][1,2,4]triazolo[4,3-f]pteridine

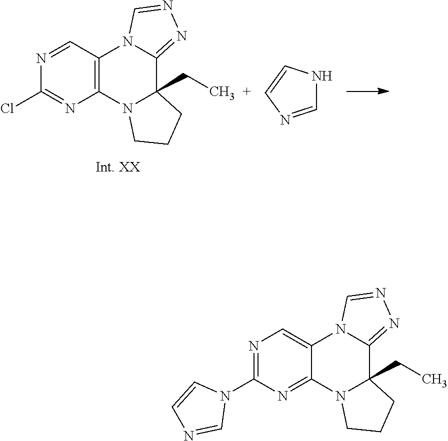

Intermediate XX (0.375 mmol) and 1H-imidazole (3.749 mmol) are combined in a sealed tube. The tube is plunged into a preheated 140° C. oil bath and stirred for 18 h. The reaction mixture is cooled to rt, diluted with DCM and washed with aqueous saturated NH₄Cl. The organic layer is dried with Na₂SO₄, filtered and concentrated. The resulting residue is purified by flash chromatography to give the title compound.

Additional compounds are prepared similarly to this method, replacing Intermediate xx with a suitable Intermediate. In some instances, where a racemic mixture results, the two enantiomers may be isolated by chiral chromatography. The following compounds are prepared:
(S)-12a-ethyl-7-(1H-imidazol-1-yl)-3-methyl-10,11,12,12a-tetrahydropyrrolo[2,1-h][1,2,4]triazolo[4,3-f]pteridine (Example 600),
(R)-4-ethyl-7-(1H-imidazol-1-yl)-5-(tetrahydro-2H-pyran-4-yl)-4,5-dihydro-[1,2,4]triazolo[4,3-f]pteridine (Example 601),
(R)-4-ethyl-7-(1H-imidazol-1-yl)-1-methyl-5-(tetrahydro-2H-pyran-4-yl)-4,5-dihydro-[1,2,4]triazolo[4,3-f]pteridine (Example 602),
(4R)-4-ethyl-7-(1H-imidazol-1-yl)-5-(tetrahydrofuran-3-yl)-4,5-dihydro-[1,2,4]triazolo[4,3-f]pteridine (Example 603),
(4R)-4-ethyl-7-(1H-imidazol-1-yl)-1-methyl-5-(tetrahydrofuran-3-yl)-4,5-dihydro-[1,2,4]triazolo[4,3-f]pteridine (Example 604),
4-ethyl-7-(1H-imidazol-1-yl)-5-phenyl-4,5-dihydro-[1,2,4]triazolo[4,3-f]pteridine (Example 605),
4-ethyl-7-(1H-imidazol-1-yl)-1-methyl-5-phenyl-4,5-dihydro-[1,2,4]triazolo[4,3-f]pteridine (Example 606),
(R)-7-(1H-imidazol-1-yl)-4-(2,2,2-trifluoroethyl)-5-(3,3,3-trifluoropropyl)-4,5-dihydro-[1,2,4]triazolo[4,3-f]pteridine and (S)-7-(1H-imidazol-1-yl)-4-(2,2,2-trifluoroethyl)-5-(3,3,3-trifluoropropyl)-4,5-dihydro-[1,2,4]triazolo[4,3-f]pteridine (Example 607),
(R)-7-(1H-imidazol-1-yl)-1-methyl-4-(2,2,2-trifluoroethyl)-5-(3,3,3-trifluoropropyl)-4,5-dihydro-[1,2,4]triazolo[4,3-f]pteridine and (S)-7-(1H-imidazol-1-yl)-1-methyl-4-(2,2,2-trifluoroethyl)-5-(3,3,3-trifluoropropyl)-4,5-dihydro-[1,2,4]triazolo[4,3-f]pteridine (Example 608),
(R)-5-(3,3-difluorocyclobutyl)-4-ethyl-7-(1H-imidazol-1-yl)-4,5-dihydro-[1,2,4]triazolo[4,3-f]pteridine (Example 609),
(R)-5-(3,3-difluorocyclobutyl)-4-ethyl-7-(1H-imidazol-1-yl)-1-methyl-4,5-dihydro-[1,2,4]triazolo[4,3-f]pteridine (Example 610),
4-ethyl-7-(1H-imidazol-1-yl)-5-(3-(pyrimidin-5-yl)phenyl)-4,5-dihydro-[1,2,4]triazolo[4,3-f]pteridine (Example 611),
4-ethyl-7-(1H-imidazol-1-yl)-1-methyl-5-(3-(pyrimidin-5-yl)phenyl)-4,5-dihydro-[1,2,4]triazolo[4,3-f]pteridine (Example 612),
5-(3-(1H-pyrazol-1-yl)phenyl)-4-ethyl-7-(1H-imidazol-1-yl)-4,5-dihydro-[1,2,4]triazolo[4,3-f]pteridine (Example 613),
5-(3-(1H-pyrazol-1-yl)phenyl)-4-ethyl-7-(1H-imidazol-1-yl)-1-methyl-4,5-dihydro-[1,2,4]triazolo[4,3-f]pteridine (Example 614),
4-ethyl-7-(1H-imidazol-1-yl)-5-(1H-pyrazol-4-yl)-4,5-dihydro-[1,2,4]triazolo[4,3-f]pteridine (Example 615), and
4-ethyl-7-(1H-imidazol-1-yl)-1-methyl-5-(1H-pyrazol-4-yl)-4,5-dihydro-[1,2,4]triazolo[4,3-f]pteridine (Example 616).
   For Examples 615 and 616, the SEM nitrogen protecting group is removed similarly to the method of Example 655. The following table provides the example number (column 1) and Intermediate (column 2) used to give the compound shown in column 3.

| Ex. No. | Int. | Compound structure |
|---|---|---|
| 600 | XX′ | 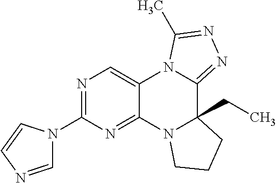 |
| 601 | M | 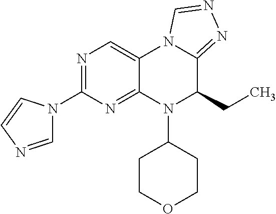 |
| 602 | M′ | 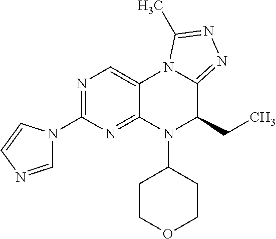 |
| 603 | N | 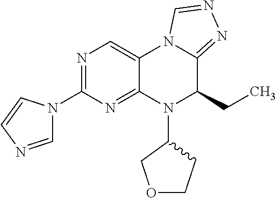 |
| 604 | N′ | 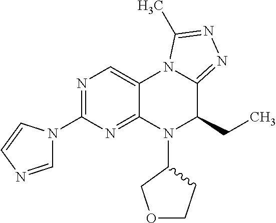 |
| 605 | CC | 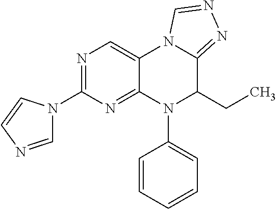 |
| 606 | CC′ | 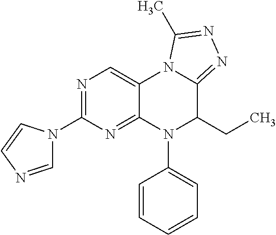 |
| 607(R) | BB | 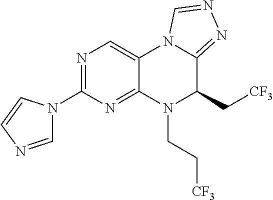 |
| 607(S) | | 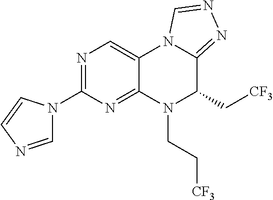 |
| 608(R) | BB′ | 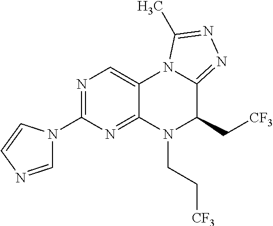 |
| 608(S) | | 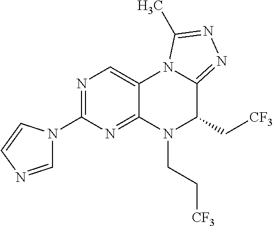 |
| 609 | V | 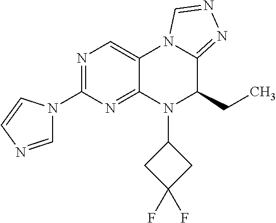 |
| 610 | V′ | 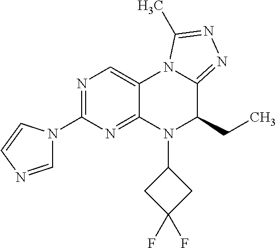 |
| 611 | MM | 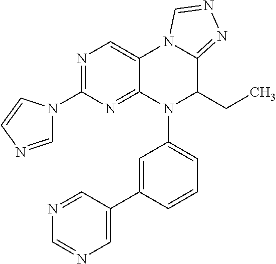 |
| 612 | MM′ | 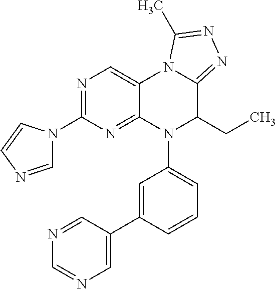 |
| 613 | NN | 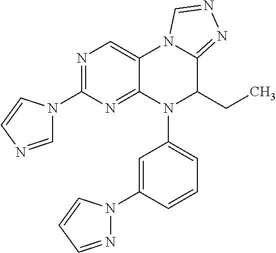 |
| 614 | NN′ | 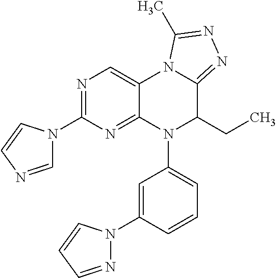 |
| 615 | KK | 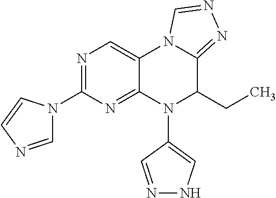 |
| 616 | KK′ | 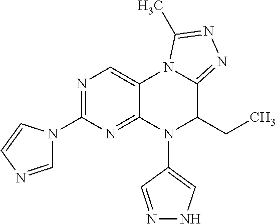 |

### Example 617 and Example 618

### Synthesis of (R)-5-cyclopentyl-7-(2-cyclopropyl-1H-imidazol-1-yl)-4-ethyl-4,5-dihydro-[1,2,4]triazolo[4,3-f]pteridine (617) and (R)-5-cyclopentyl-7-(2-cyclopropyl-1H-imidazol-1-yl)-4-ethyl-1-methyl-4,5-dihydro-[1,2,4]triazolo[4,3-f]pteridine (618)

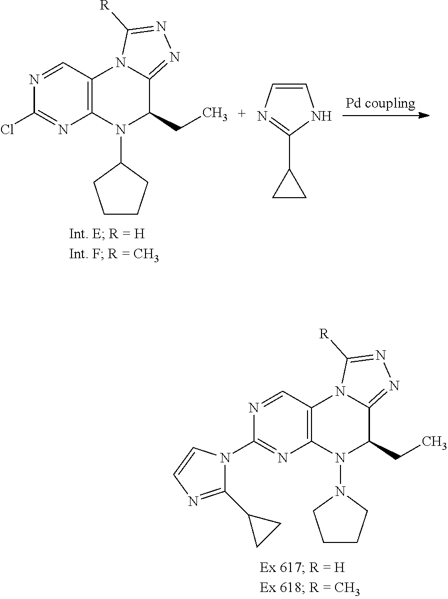

Intermediate E or Intermediate F is reacted via palladium coupling with 2-cyclopropyl-1H-imidazole (synthesized according to U.S. Pat. No. 6,610,723, column 91, Example 409, the disclosure of which is hereby incorporated by reference with respect to this compound) to provide the title compound.

### Example 619 and Example 620

### Synthesis of (R)-4-(4-ethyl-5-isopropyl-4,5-dihydro-[1,2,4]triazolo[4,3-f]pteridin-7-yl)-S-(4-fluorophenyl)isothiazole (619) and (R)-4-(4-ethyl-5-isopropyl-1-methyl-4,5-dihydro-[1,2,4]triazolo[4,3-f]pteridin-7-yl)-5-(4-fluorophenyl)isothiazole (620)

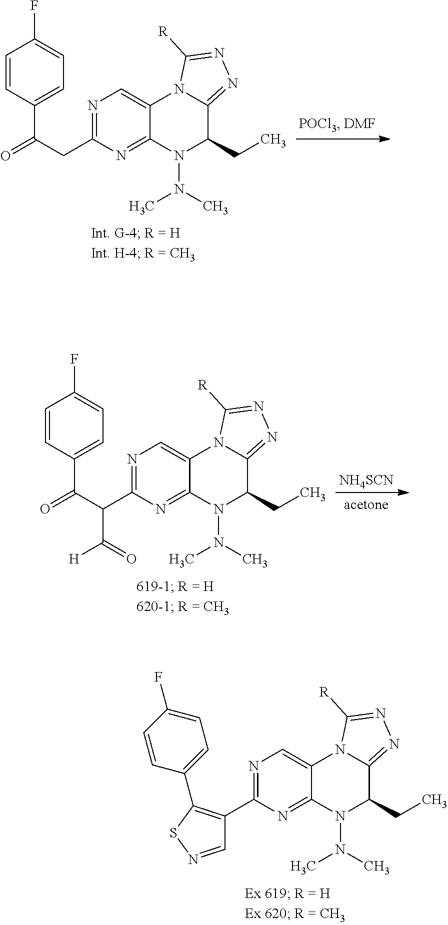

A mixture of Intermediate G-4 or H-4 (0.691 mmol) in anhydrous DMF is cooled to 0° C. under N₂ (g) inlet prior to dropwise addition of phosphorus oxychloride (1.61 mmol). The reaction mixture is warmed to rt, placed in an oil bath set at 80° C. for 4 h and then quenched with water. The mixture is partitioned between water and ethyl acetate and the organic layer is dried with sodium sulfate, filtered and concentrated to give compound 619-1 or 620-1.

To compound 619-1 or 620-1 (0.204 mmol) in anhydrous acetone, ammonium thiocyanate (0.893 mmol) is added. The reaction mixture is placed in an oil bath set at 50° C. with N₂ (g) inlet for 4 h and then cooled and concentrated, then purified by preparative HPLC to give the title compounds.
(R)-3-(4-ethyl-7-(5-(4-fluorophenyl)isothiazol-4-yl)-[1,2,4]triazolo[4,3-f]pteridin-5(4H)-yl)benzonitrile, (S)-3-(4-ethyl-7-(5-(4-fluorophenyl)isothiazol-4-yl)-[1,2,4]triazolo[4,3-f]pteridin-5(4H)-yl)benzonitrile (Example 689),
(R)-3-(4-ethyl-7-(5-(4-fluorophenyl)isothiazol-4-yl)-1-methyl-[1,2,4]triazolo[4,3-f]pteridin-5(4H)-yl)benzonitrile, (S)-3-(4-ethyl-7-(5-(4-fluorophenyl)isothiazol-4-yl)-1-methyl-[1,2,4]triazolo[4,3-f]pteridin-5(4H)-yl)benzonitrile (Example 690),
(R)-4-(4-ethyl-7-(5-(4-fluorophenyl)isothiazol-4-yl)-[1,2,4]triazolo[4,3-f]pteridin-5(4H)-yl)benzonitrile, (S)-4-(4-ethyl-7-(5-(4-fluorophenyl)isothiazol-4-yl)-[1,2,4]triazolo[4,3-f]pteridin-5(4H)-yl)benzonitrile (Example 701), and
(R)-4-(4-ethyl-7-(5-(4-fluorophenyl)isothiazol-4-yl)-1-methyl-[1,2,4]triazolo[4,3-f]pteridin-5(4H)-yl)benzonitrile, (S)-4-(4-ethyl-7-(5-(4-fluorophenyl)isothiazol-4-yl)-1-methyl-[1,2,4]triazolo[4,3-f]pteridin-5(4H)-yl)benzonitrile (Example 702),

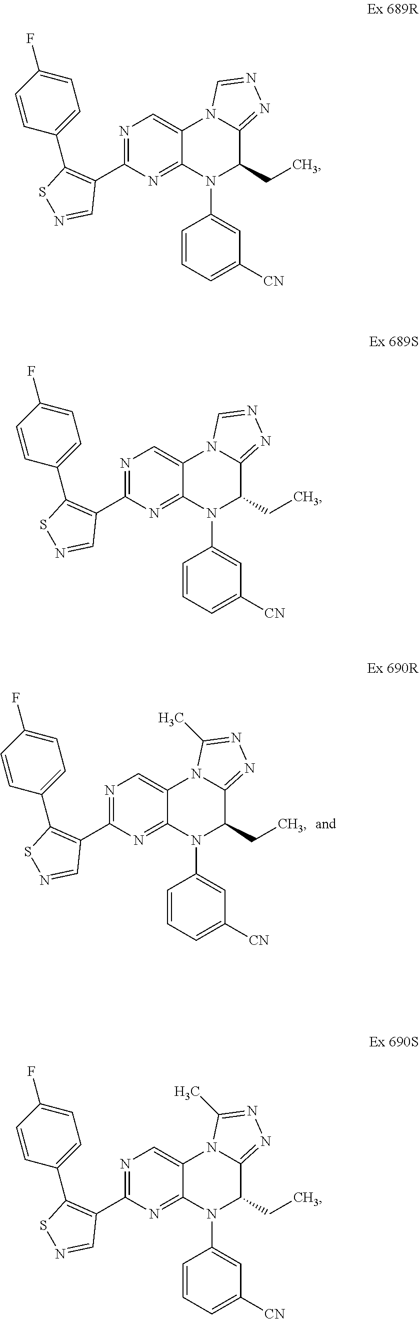

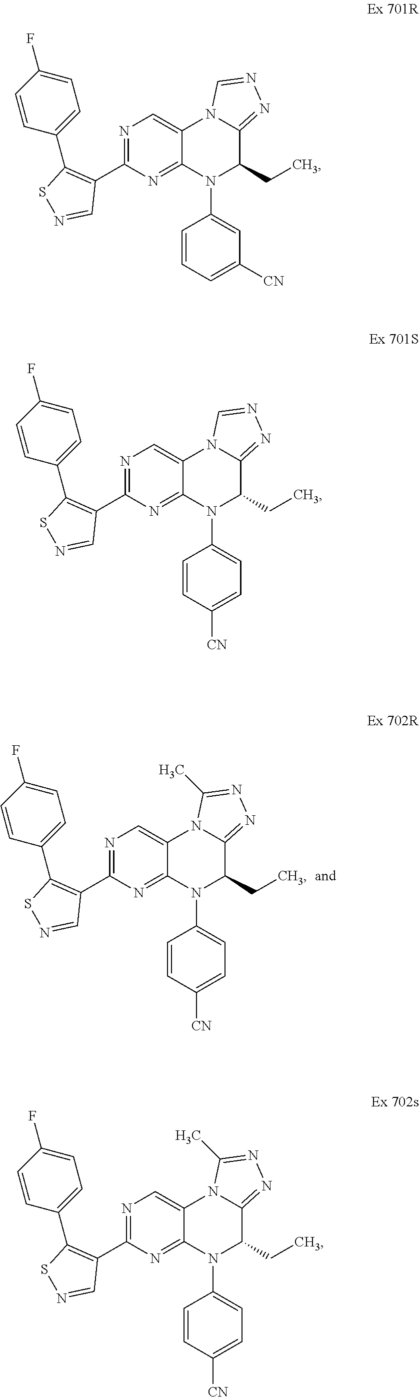

are prepared similarly, with Intermediate OO-1,00′-1, PP-3, and PP′-3 instead of Intermediate G-4 or Intermediate H-4. The resulting racemic mixture is resolved by chiral HPLC using an isocratic mixture of EtOH:Hexane (1:1, 1 mL/min) as eluent from a Chiralcel OD-H column (0.46×250 mmm) to give the isolated isomers of Example 689 and Example 690 and Examples 701 and Example 702.

### Example 621 and Example 622

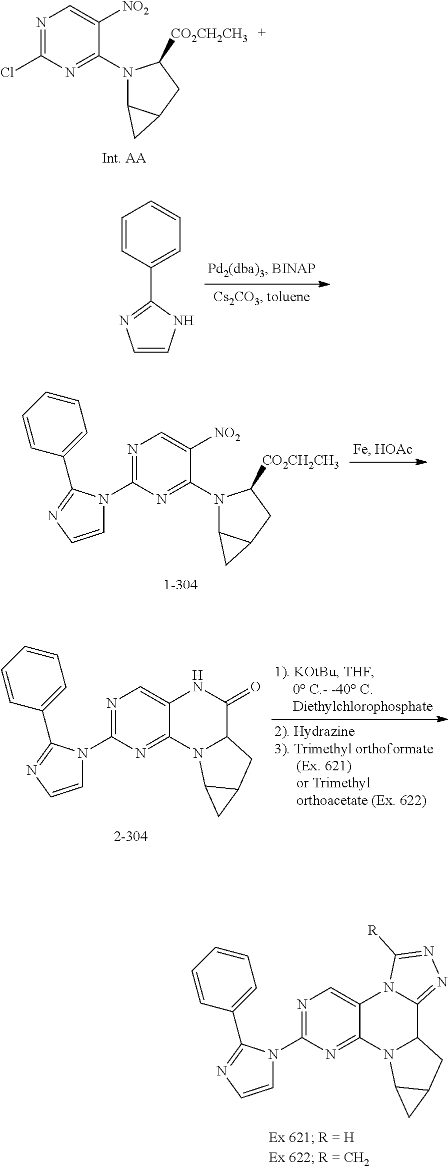

A microwave vial was charged with Intermediate AA (43.6 mg, 0.14 mmol), Pd₂(dba)₃ (25.5 mg, 0.2 eq), BINAP (43.6 mg, 0.5 eq), Cs₂CO₃ (137 mg, 3 eq), 2-phenyl-1H-imidazole (22.2 mg, 1.1 eq) and 0.5 mL of toluene. The vial was heated in a microwave at 140° C. for 60 min. The reaction mixture was diluted with EtOAc and the solid was filtered off. After evaporation of the solvent, the crude material was purified by MPLC to give compound 621-1.

Compound 621-2 was synthesized from compound 621-1 similarly to the analogous step in Example 275.

Example 621 and Example 622 are synthesized from compound 621-2 similarly to the analogous step in Example 275.

### Example 623

### Synthesis of (R)-5-cyclopentyl-4-ethyl-7-(5-(pyridin-2-yl)-1H-pyrazol-4-yl)-4,5-dihydro-[1,2,4]triazolo[4,3-f]pteridine

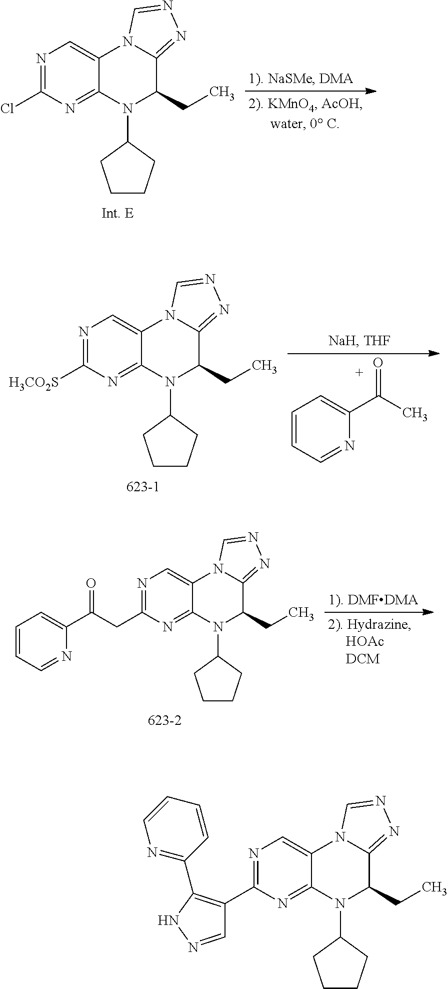

To a stirring mixture of Intermediate E (1 eq) in DMA, sodium methanethiolate (2.0 eq) is added. The reaction mixture is placed in a 150° C. preheated oil bath and stirred for 2 hr. The reaction mixture is cooled to rt and slowly diluted with ethyl ether and brine. The layers are separated. The aqueous layer is extracted 2× with ethyl ether. The combined organic layers are dried over MgSO₄, filtered, and concentrated under reduced pressure. To a stirring mixture of the crude methyl sulfide pteridine in HOAc at 0° C., a solution of KMnO₄ (2 eq) in water is added slowly over 10 min. The reaction mixture is reacted for 1 h before additional KMnO₄ (0.5 eq) in water is added. Cold water and a 10% Na₂S₂O₃ solution are added. The reaction mixture is diluted with EtOAc. The layers are separated and the aqueous layer is extracted 2× with EtOAc. The combined organic layers are dried over MgSO₄, filtered, and concentrated under reduced pressure. The resulting material is purified by MPLC to give compound 623-1.

To a stirring mixture of the compound 623-1 (1 eq) and 1-(pyridin-2-yl)ethanone (3 eq) in THF at rt, NaH (3 eq) is added in small portions. The reaction mixture is warmed to reflux for 20 min. The reaction mixture is cooled to rt and the reaction is quenched with brine and EtOAc. The layers are separated and the aqueous layer is extracted 2× with EtOAc. The organic layers are dried over MgSO₄, filtered, and concentrated under reduced pressure to give compound 623-2.

Compound 623-2 is dissolved in DMFDMA. The reaction mixture is warmed to 72° C. for 45 min, then concentrated and the residue dissolved in DCM. Hydrazine (3 drops) and HOAc (3 drops) are added to the stirring mixture. The reaction mixture is warmed to reflux for 10 min, then cooled to rt and slowly quenched with a saturated NaHCO₃ solution. The aqueous layer is extracted 2× with DCM. The organic layers are dried over MgSO₄, filtered, and concentrated, and the residue is purified by preparative HPLC to give the title compound.

Additional compounds are prepared similarly to this method, optionally replacing Intermediate E with a suitable Intermediate, and/or replacing 1-(pyridin-2-yl)ethanone with a suitable ketone reactant. In some instances, where a racemic mixture results, the two enantiomers may be isolated by chiral chromatography. The following compounds are prepared:
(R)-5-cyclopentyl-4-ethyl-1-methyl-7-(5-(pyridin-2-yl)-1H-pyrazol-4-yl)-4,5-dihydro-[1,2,4]triazolo[4,3-f]pteridine (Example 624),
(R)-4-(4-(5-cyclopentyl-4-ethyl-4,5-dihydro-[1,2,4]triazolo[4,3-f]pteridin-7-yl)-1H-pyrazol-5-yl)thiazole (Example 625),
(R)-4-(4-(5-cyclopentyl-4-ethyl-1-methyl-4,5-dihydro-[1,2,4]triazolo[4,3-f]pteridin-7-yl)-1H-pyrazol-5-yl)thiazole (Example 626),
(R)-2-(4-(4-ethyl-4-methyl-5-(3,3,3-trifluoropropyl)-4,5-dihydro-[1,2,4]triazolo[4,3-f]pteridin-7-yl)-1H-pyrazol-5-yl)thiazole (Example 627),
(R)-2-(4-(4-ethyl-1,4-dimethyl-5-(3,3,3-trifluoropropyl)-4,5-dihydro-[1,2,4]triazolo[4,3-f]pteridin-7-yl)-1H-pyrazol-5-yl)thiazole (Example 628),
(R)-7-(5-(2,4-difluorophenyl)-1H-pyrazol-4-yl)-4-ethyl-5-isopropyl-4,5-dihydro-[1,2,4]triazolo[4,3-f]pteridine (Example 629),
(R)-7-(5-(2,4-difluorophenyl)-1H-pyrazol-4-yl)-4-ethyl-5-isopropyl-1-methyl-4,5-dihydro-[1,2,4]triazolo[4,3-f]pteridine (Example 630),
(R)-4-ethyl-5-isopropyl-7-(5-(pyridin-2-yl)-1H-pyrazol-4-yl)-4,5-dihydro-[1,2,4]triazolo[4,3-f]pteridine (Example 631),
(R)-4-ethyl-5-isopropyl-1-methyl-7-(5-(pyridin-2-yl)-1H-pyrazol-4-yl)-4,5-dihydro-[1,2,4]triazolo[4,3-f]pteridine (Example 632),
(R)-4-(4-(4-ethyl-5-isopropyl-4,5-dihydro-[1,2,4]triazolo[4,3-f]pteridin-7-yl)-1H-pyrazol-5-yl)thiazole (Example 633), and
(R)-4-(4-(4-ethyl-5-isopropyl-1-methyl-4,5-dihydro-[1,2,4]triazolo[4,3-f]pteridin-7-yl)-1H-pyrazol-5-yl)thiazole (Example 634),
(R)-2-(4-(4-ethyl-5-(tetrahydro-2H-pyran-4-yl)-4,5-dihydro-[1,2,4]triazolo[4,3-f]pteridin-7-yl)-1H-pyrazol-5-yl)thiazole (Example 643), and
(R)-2-(4-(4-ethyl-1-methyl-5-(tetrahydro-2H-pyran-4-yl)-4,5-dihydro-[1,2,4]triazolo[4,3-f]pteridin-7-yl)-1H-pyrazol-5-yl)thiazole (Example 644).
   The following table provides the example number (column 1), Intermediate (column 2), and ketone reactant (column 3) used to give the compound shown in column 4.

| Ex. No. | Int. | Ketone reactant | Compound structure |
|---|---|---|---|
| 624 | F | 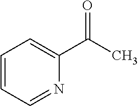 | 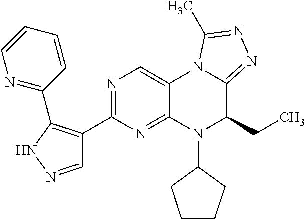 |
| 625 | E | 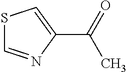 | 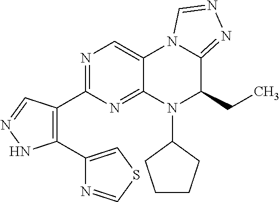 |
| 626 | F | | 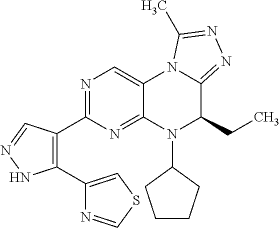 |
| 627 | ZZ | 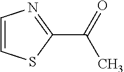 | 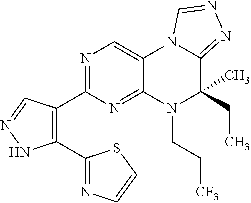 |
| 628 | ZZ′ | | 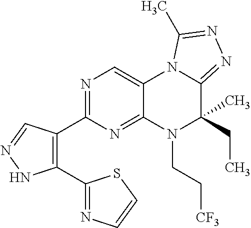 |
| 629 | G | 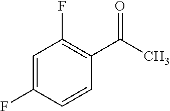 | 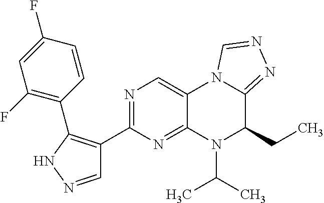 |
| 630 | H | | 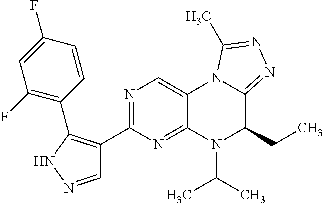 |
| 631 | G | 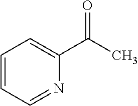 | 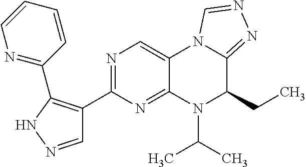 |
| 632 | H | | 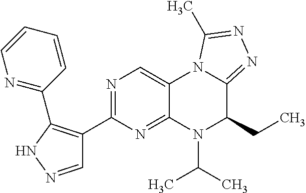 |
| 633 | G | 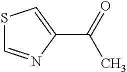 | 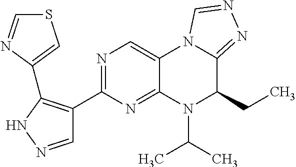 |
| 634 | H | | 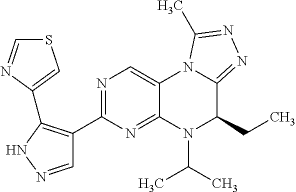 |
| 643 | M | 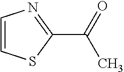 | 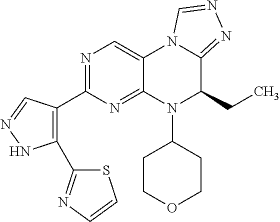 |
| 644 | M′ | | 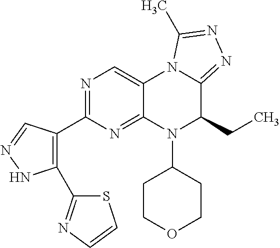 |

### Example 635 and Example 636

### Synthesis of (10R,12aS)-10,12a-diethyl-7-(2-phenyl-1H-imidazol-1-yl)-10,11,12,12a-tetrahydropyrrolo[2,1-h][1,2,4]triazolo[4,3-f]pteridine (635) and (10R,12aS)-10,12a-diethyl-3-methyl-7-(2-phenyl-1H-imidazol-1-yl)-10,11,12,12a-tetrahydropyrrolo[2,1-h][1,2,4]triazolo[4,3-f]pteridine (636)

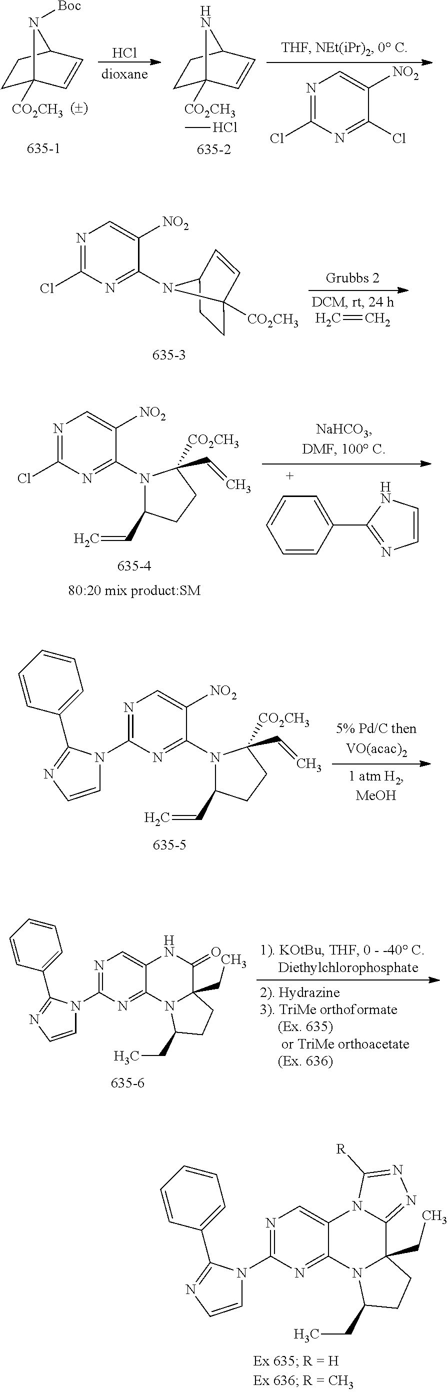

2,4-Dichloro-5-nitropyrimidine and (+)-7-tert-butyl 1-methyl 7-azabicyclo[2.2.1]hept-5-ene-1,7-dicarboxylate (Compound 635-1, 1.1 g, 4.5 mmol, prepared according to the literature method: Carreras, J. et al. Org. Lett. 2007, 9, 1235-1238) was dissolved in 4 N HCl in 5 mL of dioxane at 0° C., then allowed to warm to rt for 1 h. The mixture was diluted with diethyl ether, and the resulting solid filtered through a sintered glass funnel, and washed with a few mL of cold diethyl ether to give compound 635-2 as a crude off-white solid (700 mg, 82%).

Compound 635-2 (700 mg, 3.7 mmol) was suspended in 7 mL of dry THF at 0° C., and 2,4-dichloro-5-nitropyrimidine (AK Scientific, 725 mg, 3.74 mmol) was added. Diisopropylethylamine (1.36 mL, 7.77 mmol) was added dropwise by syringe to this mixture with stirring. After 1 h, the reaction mixture was concentrated under reduced pressure, and the residue purified by flash chromatography (EtOAc/hexanes elution) to give compound 635-3 (1.14 g, 99%): LCMS: 311.0 m/z (M+H)⁺.

Compound 635-4 was synthesized similarly to the literature procedure: Heterocycles 2006, 68, 2079. Compound 635-3 (86 mg, 0.28 mmol) was dissolved in 14 mL of dry DCM, which was saturated in ethylene (g). Second generation Grubbs′ catalyst[1,3-bis(2,4,6-trimethylphenyl)-2-imidazolidinylidene]dichloro(phenylmethylene)-(tricyclohexylphosphine)ruthenium] (30.1 mg, 0.035 mmol) was added, and the reaction was performed under an atmosphere of ethylene, with vigorous stirring at rt. After 27 h, the reaction was concentrated, and the mixture purified by flash chromatography (0-30% EtOAc/hexanes elution) to give a mixture of 635-3 and 635-4 (LCMS: 339.1 m/z (M+H)⁺).

Compound 635-4 (94 mg, 0.278 mmol, some 3-309) was dissolved in 1 mL of dry DMF, and NaHCO₃ (73 mg, 0.869 mmol) and 2-phenyl-1H-imidazole (118 mg, 0.821 mmol) were added. This mixture was heated to 100° C. for 15 h, then the solvents were removed, and the residue purified by flash chromatography (50-100% EtOAc/hexanes elution) to give compound 635-5 (67 mg, 54%): LCMS: 447.2 m/z (M+H)⁺.

According to the method outlined in WO 2009/019205, p. 13, compound 635-5 (67 mg, 0.15 mmol) was dissolved in 1 mL of MeOH, and 5% palladium on carbon (41 mg) was added. This was placed under a H₂ atm with stirring at rt. After 3 h, vanadyl acetylacetonate (27 mg, 0.10 mmol) was added, and the H₂ atm replaced. This was stirred at rt for 16 h, then the reaction mixture was filtered through diatomaceous earth, washed with MeOH, and the filtrate concentrated under reduced pressure to give compound 635-6. LCMS: 389.2 m/z (M+H)⁺.

Compound 635-6 is reacted similarly to the analogous step of Example 13, to give Example 635 or Example 636.

(10R,12aS)-10,12a-diethyl-7-(2-methyl-1H-imidazol-1-yl)-10,11,12,12a-tetrahydropyrrolo[2,1-h][1,2,4]triazolo[4,3-f]pteridine (Example 645) and (10R,12aS)-10,12a-diethyl-3-methyl-7-(2-methyl-1H-imidazol-1-yl)-10,11,12,12a-tetrahydropyrrolo[2,1-h][1,2,4]triazolo[4,3-f]pteridine (Example 646)

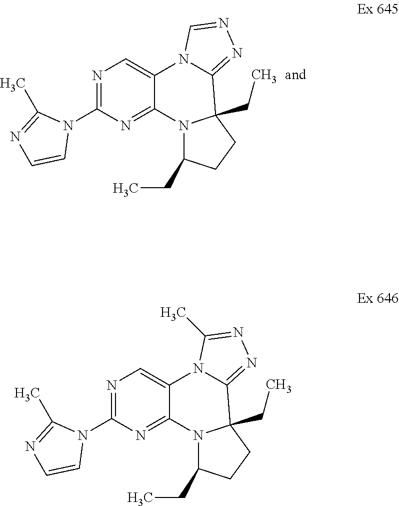

are prepared similarly, with 2-methyl-1H-imidazole instead of 2-phenyl-1H-imidazole in the reaction with compound 635-4.

(10R,12aS)-10,12a-diethyl-7-(1H-imidazol-1-yl)-10,11,12,12a-tetrahydropyrrolo[2,1-h][1,2,4]triazolo[4,3-f]pteridine (Example 657) and (10R,12aS)-10,12a-diethyl-3-methyl-7-(1H-imidazol-1-yl)-10,11,12,12a-tetrahydropyrrolo[2,1-h][1,2,4]triazolo[4,3-f]pteridine (Example 658)

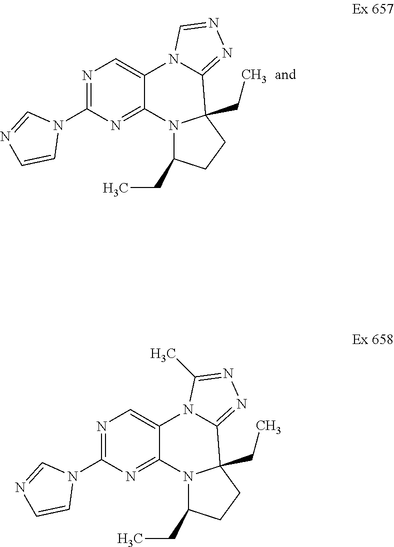

are prepared similarly, with 1H-imidazole instead of 2-phenyl-1H-imidazole in the reaction with compound 635-4.

### Example 637 and Example 638

### Synthesis of (S)-12a-ethyl-7-(1H-imidazol-1-yl)-12,12a-dihydropyrrolo[2,1-h][1,2,4]triazolo[4,3-f]pteridin-10(11H)-one (637) and (S)-12a-ethyl-7-(1H-imidazol-1-yl)-3-methyl-12,12a-dihydropyrrolo[2,1-h][1,2,4]triazolo[4,3-f]pteridin-10(11H)-one (638)

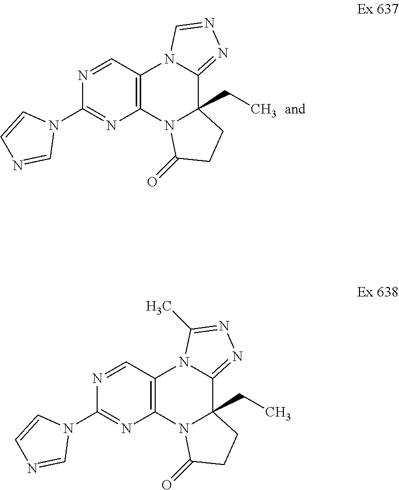

Intermediate xx is added to a solution of sodium periodate (8.285 mmol) and ruthenium(III) chloride hydrate (0.165 mmol) in water. The reaction mixture is stirred at rt for 72 h, then is diluted with 20 mL of iPrOH, stirred for 1 h, and concentrated. The resulting residue is dissolved in EtOAc and washed with water. The organic layer is dried with Na₂SO₄, filtered and concentrated. The resulting residue is purified by flash chromatography (30% EtOAc in hexanes). The resulting residue is dissolved in AcOH and iron (0.882 mmol) is added. The reaction mixture is fitted with a reflux condenser, is plunged into a preheated 90° C. oil bath, and is stirred for 1 h. The reaction mixture is cooled to rt, diluted with EtOAc, washed with water, saturated NaHCO₃, dried with Na₂SO₄, filtered and concentrated. The resulting residue is reacted similarly to the last step of Example 13 with either trimethyl orthoformate or trimethy orthoacetate to give the title compounds.

### Example 639 and Example 640

### Synthesis of (R)-5-(4-ethyl-5-isopropyl-4,5-dihydro-[1,2,4]triazolo[4,3-f]pteridin-7-yl)-4-(4-fluorophenyl)thiazol-2-amine (639) and (R)-5-(4-ethyl-5-isopropyl-1-methyl-4,5-dihydro-[1,2,4]triazolo[4,3-f]pteridin-7-yl)-4-(4-fluorophenyl)thiazol-2-amine (640)

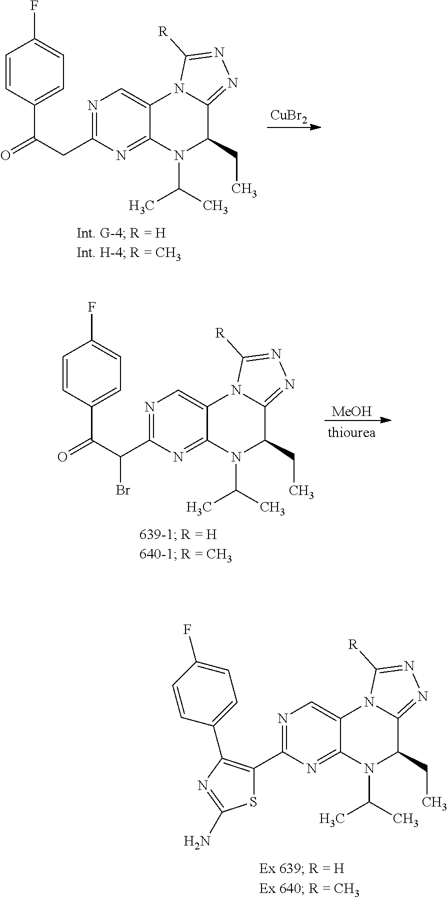

To a solution of Intermediate G-4 or Intermediate H-4 (0.891 mmol) in ethyl acetate, copper (II) bromide is added. The reaction mixture is placed in an oil bath set at 50° C. for 1.5 h. The mixture is partitioned between saturated NaHCO₃ and ethyl acetate and the organic layer is dried with sodium sulfate, filtered and concentrated to give compound 639-1 or 640-1.

To a solution of compound 639-1 or 640-1 (0.347 mmol) in methanol, thiourea (0.342 mmol) is added. The reaction mixture is place in an oil bath set at 90° C. for 2 h and then concentrated and purified by preparative HPLC to give the title compounds.

### Example 641 and Example 642

### Synthesis of (R)-5-(4-ethyl-5-isopropyl-4,5-dihydro-[1,2,4]triazolo[4,3-f]pteridin-7-yl)-4-(4-fluorophenyl)thiazole (641) and (R)-5-(4-ethyl-5-isopropyl-1-methyl-4,5-dihydro-[1,2,4]triazolo[4,3-f]pteridin-7-yl)-4-(4-fluorophenyl)thiazole (642)

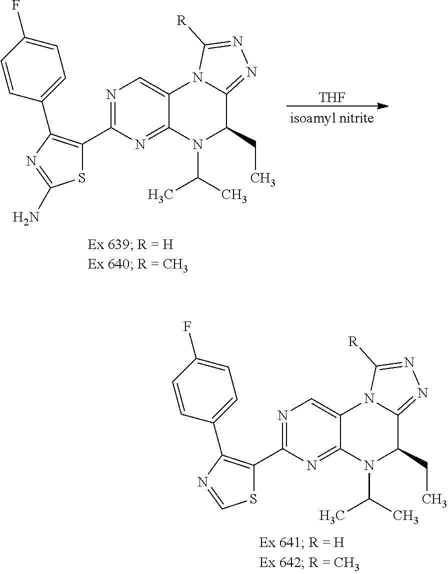

To a solution of Example 639 or Example 640 (0.324 mmol) in anhydrous THF, isoamyl nitrite (0.751 mmol) is added. The reaction mixture is place in an oil bath set at 85° C. for 2 h and then concentrated and purified by preparative HPLC to give the title compounds.

### Example 647 and Example 648

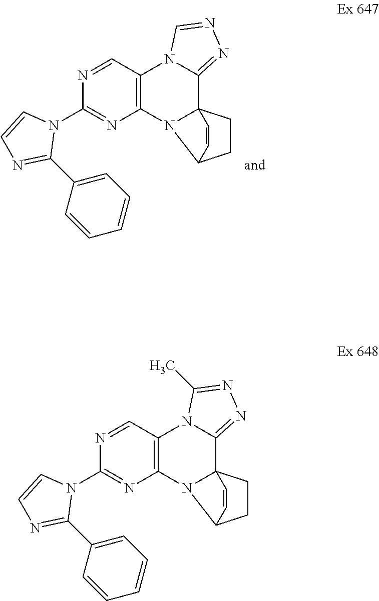

Example 647 and Example 648 are prepared similarly to the methods described in Example 13, with Compound 635-3 from Example 635 instead of Intermediate E-0 and with 2-phenyl-1H-imidazole instead of 1H-imidazole in the first step.

### Example 649 and Example 650

### Synthesis of (R)-4-ethyl-7-(2-(4-fluorophenyl)-1H-imidazol-1-yl)-5-(oxetan-3-yl)-4,5-dihydro-[1,2,4]triazolo[4,3-f]pteridine (649) and (R)-4-ethyl-7-(2-(4-fluorophenyl)-1H-imidazol-1-yl)-1-methyl-5-(oxetan-3-yl)-4,5-dihydro-[1,2,4]triazolo[4,3-f]pteridine (650)

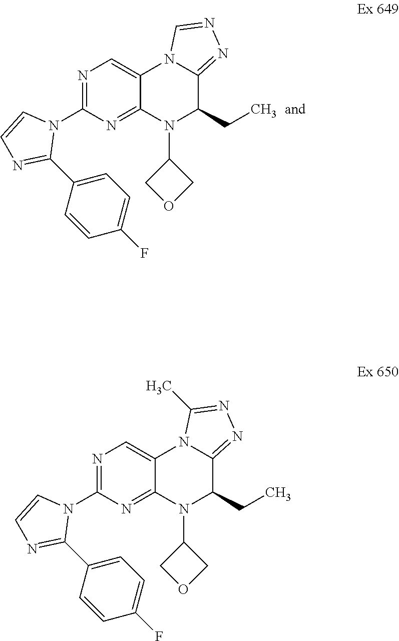

To a stirring mixture of Intermediate RR-1 (0.338 mmol) in DMSO, 2-(4-fluorophenyl)-1H-imidazole (0.67 mmol) is added. The reaction mixture is placed in a 120° C. oil bath for 2 h. The crude mixture is directly loaded and purified by silica gel chromatography to give the coupled nitro ester. To a stirring mixture of the coupled nitro ester in MeOH, Pt/C (42 mg) is added and the reaction mixture is placed under 1 atm of hydrogen for 2 h. The hydrogen balloon is removed and VO(acac)₂ is added. This reaction mixture is placed under 1 atm of hydrogen overnight. The crude mixture is filtered through a plug of Celite and the plug is washed several times with EtOAc. The filtrate is concentrated under reduced pressure. This cyclized compound is then reacted similarly to the final step of Example 13 with either trimethyl orthoformate or trimethylorthoacetate to give the title compounds.

(R)-4-ethyl-5-(oxetan-3-yl)-7-(2-phenyl-1H-imidazol-1-yl)-4,5-dihydro-[1,2,4]triazolo[4,3-f]pteridine (Example 659) and (R)-4-ethyl-1-methyl-5-(oxetan-3-yl)-7-(2-phenyl-1H-imidazol-1-yl)-4,5-dihydro-[1,2,4]triazolo[4,3-f]pteridine (Example 660)

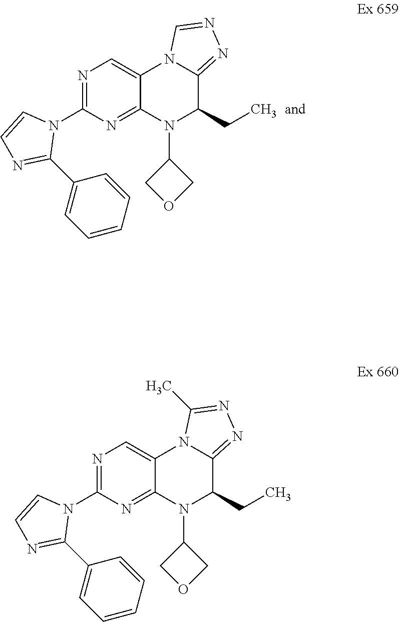

are prepared similarly, with 2-phenyl-1H-imidazole instead of 2-(4-fluorophenyl)-1H-imidazole in the first step.

(R)-7-(2-(3,4-difluorophenyl)-1H-imidazol-1-yl)-4-ethyl-5-(oxetan-3-yl)-4,5-dihydro-[1,2,4]triazolo[4,3-f]pteridine (Example 667) and (R)-7-(2-(3,4-difluorophenyl)-1H-imidazol-1-yl)-4-ethyl-1-methyl-5-(oxetan-3-yl)-4,5-dihydro-[1,2,4]triazolo[4,3-f]pteridine (Example 668)

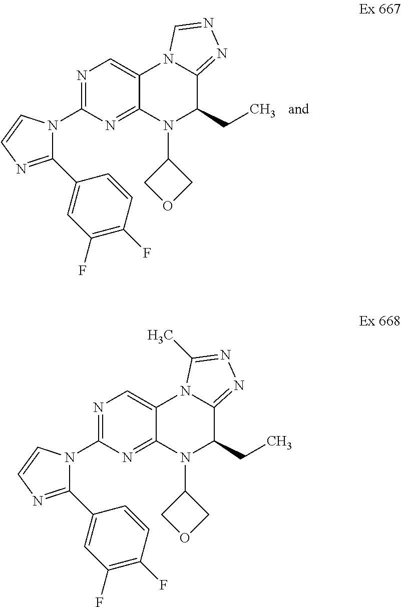

are prepared similarly, with 2-(3,4-difluorophenyl)-1H-imidazole instead of 2-(4-fluorophenyl)-1H-imidazole in the first step.

### Example 651 and Example 652

### Synthesis of 7-(1H-imidazol-1-yl)-12a-(2,2,2-trifluoroethyl)-10,11,12,12a-tetrahydropyrrolo[2,1-h][1,2,4]triazolo[4,3-f]pteridine (651) and 7-(1H-imidazol-1-yl)-3-methyl-12a-(2,2,2-trifluoroethyl)-10,11,12,12a-tetrahydropyrrolo[2,1-h][1,2,4]triazolo[4,3-f]pteridine (652)

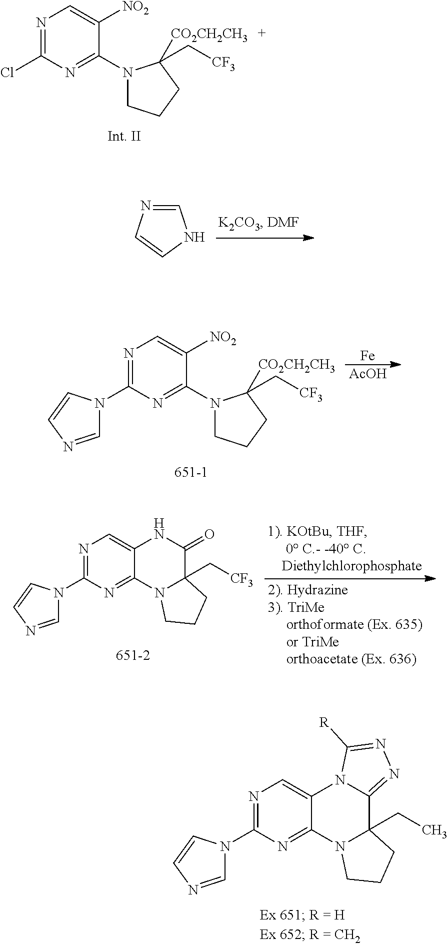

A mixture of Intermediate II (150 mg, 0.39 mmol), 1H-imidazole (40 mg, 0.59 mmol), K₂CO₃ (108 mg, 0.79 mmol) and 5 mL of DMF was heated at 50° C. for 3 h. The mixture was partitioned between 20 mL of water and 30 mL of DCM. The organic layer was washed by water (2×25 mL), dried over Na₂SO₄ and evaporated. This was purified by flash column silica chromatography (PE: EtOAc=50%:50%) to give compound 651-1. LCMS: m/z=415.1 [M+1]⁺.

Compound 651-2 was synthesized from compound 651-1 similarly to the analogous step in Example 275. LCMS: 339.1 m/z (M+H)⁺.

The title compounds are synthesized from compound 651-2 similarly to the analogous step in Example 275.

7-(2-phenyl-1H-imidazol-1-yl)-12a-(2,2,2-trifluoroethyl)-10,11,12,12a-tetrahydropyrrolo[2,1-h][1,2,4]triazolo[4,3-f]pteridine (Example 663) and 3-methyl-7-(2-phenyl-1H-imidazol-1-yl)-12a-(2,2,2-trifluoroethyl)-10,11,12,12a-tetrahydropyrrolo[2,1-h][1,2,4]triazolo[4,3-f]pteridine (Example 664)

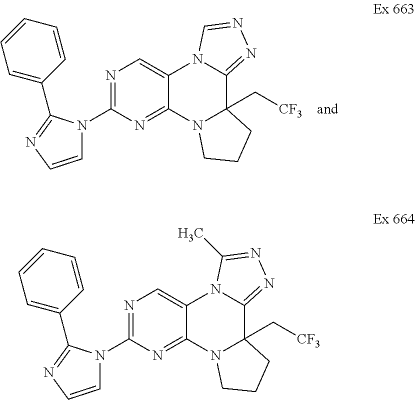

are prepared similarly, with 2-phenyl-1H-imidazole instead of 1H-imidazole in the first step.

### Example 653 and Example 654

### Synthesis of (R)-2-(5-cyclopentyl-4-ethyl-4,5-dihydro-[1,2,4]triazolo[4,3-f]pteridin-7-yl)thiazole (653) and (R)-2-(5-cyclopentyl-4-ethyl-1-methyl-4,5-dihydro-[1,2,4]triazolo[4,3-f]pteridin-7-yl)thiazole (654)

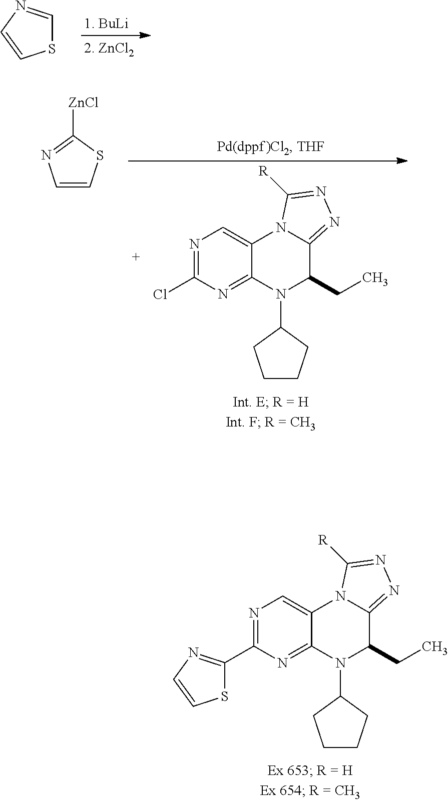

To a solution of thiazole (5 eq) in dry THF, BuLi (5 eq) is added dropwise at −78° C. and this is stirred at −78° C. for 30 min. ZnCl₂ (1M in ether, 5 eq) is added and stirred at 0° C. for 30 min, then Intermediate E or Intermediate F (1 eq) and Pd(dppf)Cl₂ (0.1 eq) are added. The reaction is heated to 70° C. for 16 h; then the mixture is diluted with EtOAc, washed with brine and concentrated. The residue is purified by silica gel flash chromatography to give the title compounds.

### Example 655 and Example 656

### Synthesis of 4-ethyl-7-(2-(4-fluorophenyl)-1H-imidazol-1-yl)-5-(1H-pyrazol-4-yl)-4,5-dihydro-[1,2,4]triazolo[4,3-f]pteridine (655) and 4-ethyl-7-(2-(4-fluorophenyl)-1H-imidazol-1-yl)-1-methyl-5-(1H-pyrazol-4-yl)-4,5-dihydro-[1,2,4]triazolo[4,3-f]pteridine (656)

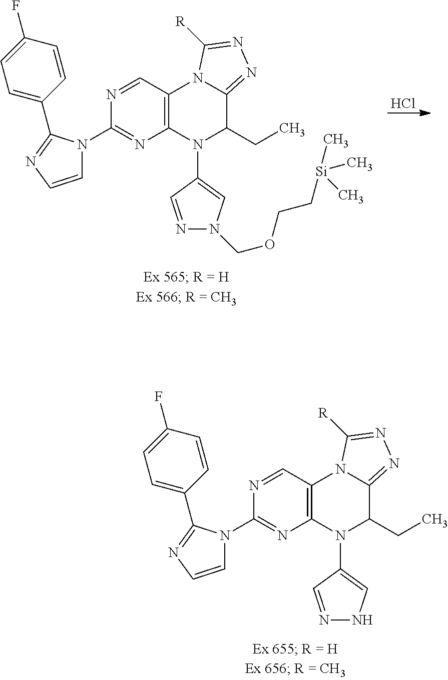

HCl (4 N solution in dioxane) is added to a solution of Example 565 or Example 566 (0.0893 mmol) in methanol and the resulting solution is stirred at 60° C. for 2 hours. The mixture is concentrated under vacuum and purified by HPLC to give the title compounds. The racemic mixture can be separated into the R or S isomers for each of these examples, e.g. by chiral HPLC, using ChiralPak AD (2×25 cm) column eluted with Ethanol: Hexane (2:3, 1 mL/min) solvent mixture.

### Example 661 and Example 662

### Synthesis of (R)-5-(4-ethyl-5-isopropyl-4,5-dihydro-[1,2,4]triazolo[4,3-f]pteridin-7-yl)-4-(1H-pyrazol-5-yl)thiazol-2-amine (661) and (R)-5-(4-ethyl-5-isopropyl-1-methyl-4,5-dihydro-[1,2,4]triazolo[4,3-f]pteridin-7-yl)-4-(1H-pyrazol-5-yl)thiazol-2-amine (662)

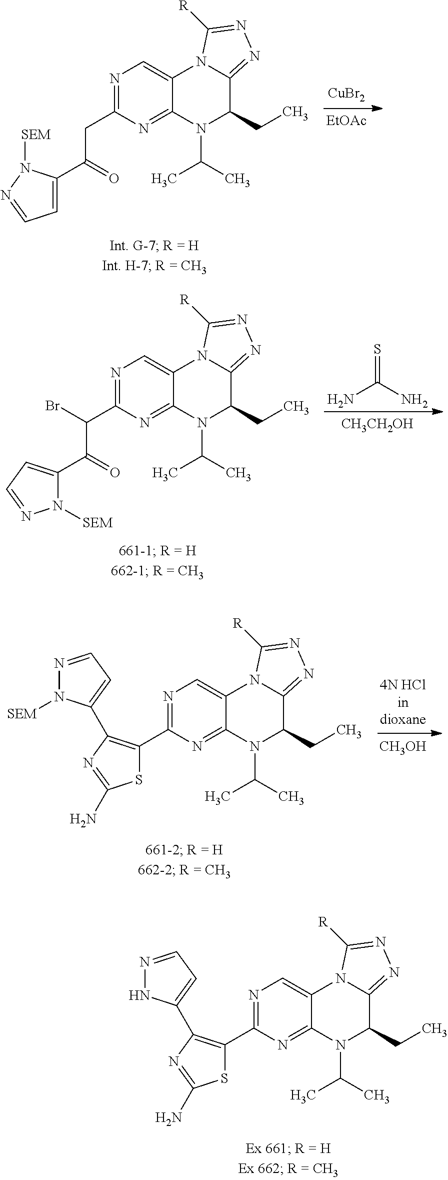

Intermediate G-7 or Intermediate H-7 is brominated similarly to the CuBr₂ procedure found in Example 475 to give compound 661-1 or 662-1.

To a solution of compound 661-1 or 662-1 (1.29 mmol) in methanol, thiourea (1.68 mmol) is added. The reaction mixture is placed in an oil bath set at 90° C. for 1 h. The reaction is quenched with water and extracted with EtOAc. The organic phase is collected, dried with sodium sulfate, filtered and concentrated to give compound 661-2 or 662-2.

A solution of compound 661-2 or 662-2 (0.165 mmol) in methanol and 4M HCl in dioxane is placed in an oil bath set at 65° C. under condenser for 1.5 h, then cooled and concentrated. The resulting material is purified by preparative HPLC to give the title compounds.

### Example 665 and Example 666

### Synthesis of 2-(4-010R,12aS)-10,12a-diethyl-10,11,12,12a-tetrahydropyrrolo[2,1-h][1,2,4]triazolo[4,3-f]pteridin-7-yl)-1H-pyrazol-5-yl)thiazole (665) and 2-(4-((10R,12aS)-10,12a-diethyl-3-methyl-10,11,12,12a-tetrahydropyrrolo[2,1-h][1,2,4]triazolo[4,3-f]pteridin-7-yl)-1H-pyrazol-5-yl)thiazole (666)

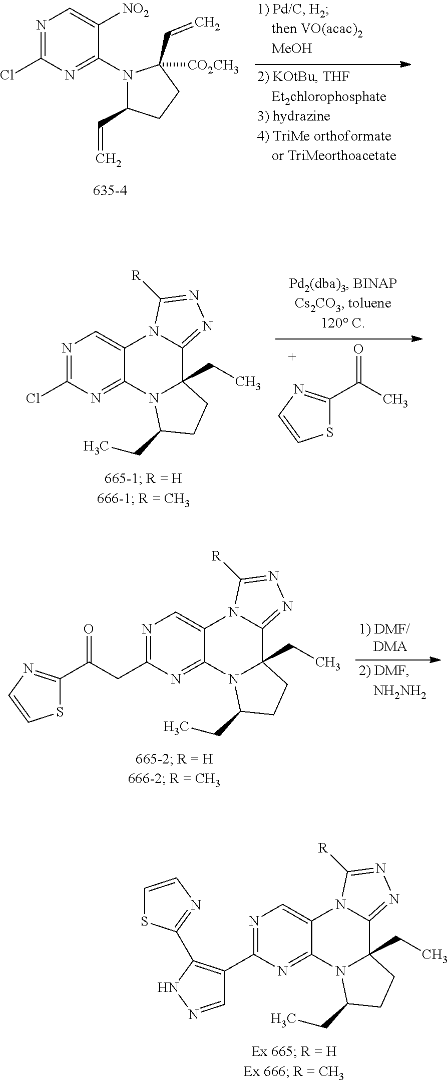

Compound 635-4 (see Example 635) is reduced and cyclized similarly to the Pd/C hydrogenation and VO(acac)₂ conditions used in Example 635 to convert compound 635-5 to 635-6, and the fused triazole formed similarly to the conversion of compound 635-6 or 636-6 to Example 635 or 636 to give compound 665-1 or 666-1.

Compound 665-1 or 666-1 is reacted similarly to the Pd coupling conditions described, for example, in the synthesis of Intermediate E-1, with 1-(thiazol-2-yl)ethanone instead of acetophenone, to give compound 665-2 or 666-2, which is then treated similarly to the conditions described in Example 457 to give the title compounds.

4-(4-((10R,12aS)-10,12a-diethyl-10,11,12,12a-tetrahydropyrrolo[2,1-h][1,2,4]triazolo[4,3-f]pteridin-7-yl)-1H-pyrazol-5-yl)thiazole (Example 677) and 4-(4-((10R,12aS)-10,12a-diethyl-3-methyl-10,11,12,12a-tetrahydropyrrolo[2,1-h][1,2,4]triazolo[4,3-f]pteridin-7-yl)-1H-pyrazol-5-yl)thiazole (Example 678)

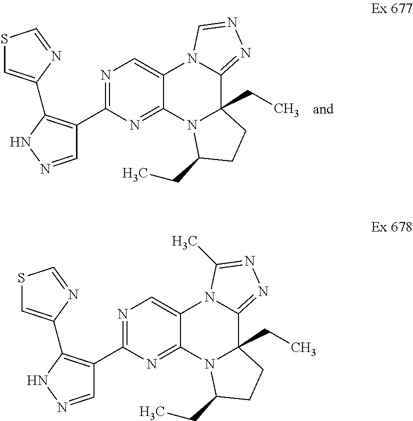

are prepared similarly, with 1-(thiazol-4-yl)ethanone instead of 1-(thiazol-2-yl)ethanone.

### Example 669 and Example 670

### Synthesis of (R)-2-bromo-5-(4-ethyl-5-isopropyl-4,5-dihydro-[1,2,4]triazolo[4,3-f]pteridin-7-yl)-4-(4-fluorophenyl)thiazole (669) and (R)-2-bromo-5-(4-ethyl-5-isopropyl-1-methyl-4,5-dihydro-[1,2,4]triazolo[4,3-f]pteridin-7-yl)-4-(4-fluorophenyl)thiazole (670)

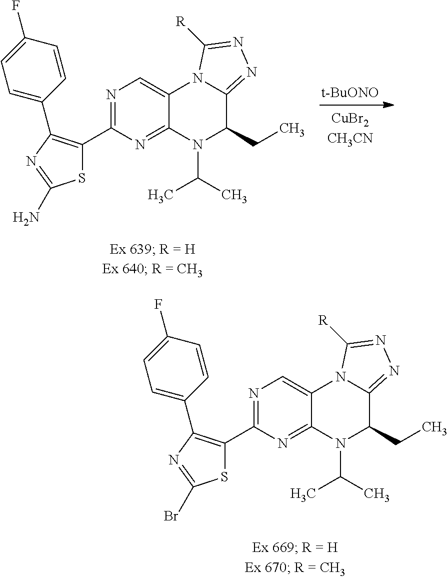

To a solution of copper (II) bromide (1.916 mmol) in anhydrous acetonitrile, t-butyl nitrite (0.926 mmol) is slowly added while stirring under N₂ (g) inlet at rt. The reaction mixture is placed in an oil bath set at 60° C. under condenser with N₂ (g) inlet. A solution of Example 639 or Example 640 (0.633 mmol) in anhydrous acetonitrile is added slowly and stirred for 1.5 h. The reaction is cooled and quenched with 1N NaOH and extracted with EtOAc. The organic phase is collected, dried with sodium sulfate, filtered and concentrated. The resulting material is purified by preparative HPLC to provide the title compounds.

### Example 671 and Example 672

### Synthesis of (R)-5-(4-ethyl-5-isopropyl-4,5-dihydro-[1,2,4]triazolo[4,3-f]pteridin-7-yl)-4-(1H-pyrazol-5-yl)thiazole (671) and (R)-5-(4-ethyl-5-isopropyl-1-methyl-4,5-dihydro-[1,2,4]triazolo[4,3-f]pteridin-7-yl)-4-(1H-pyrazol-5-yl)thiazole (672)

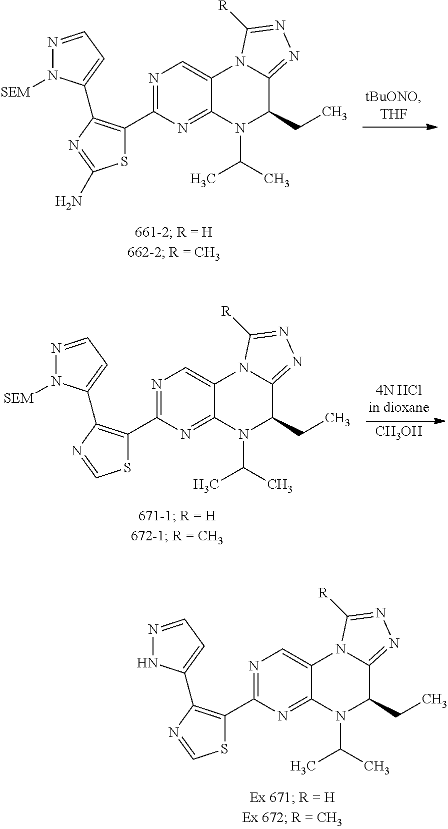

To a solution of compound 661-2 or 662-2 (0.511 mmol, see Example 661/662) in anhydrous THF, t-butyl nitrite (0.842 mmol) is added. The reaction is placed in an oil bath set at 60° C. under condenser with N₂ (g) inlet. The reaction mixture is cooled after 1 h and concentrated to give compound 671-1 or 672-1.

A solution of compound 671-1 or 672-1 (0.642 mmol) is dissolved in methanol and 4M HCl in dioxane and placed in an oil bath set at 65° C. under condenser for 1.5 h, then cooled and concentrated. The resulting material is purified by preparative HPLC to give the title compounds.

5-(4-ethyl-5-(1-methyl-1H-pyrazol-4-yl)-4,5-dihydro-[1,2,4]triazolo[4,3-f]pteridin-7-yl)-4-(1H-pyrazol-5-yl)thiazole (Example 749) and 5-(4-ethyl-1-methyl-5-(1-methyl-1H-pyrazol-4-yl)-4,5-dihydro-[1,2,4]triazolo[4,3-f]pteridin-7-yl)-4-(1H-pyrazol-5-yl)thiazole (Example 750),

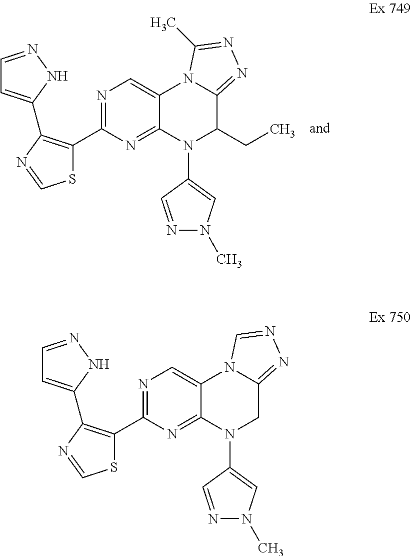

are prepared similarly, where Example 661-2 and 662-2 are substituted by reacting Intermediate KK-5 or KK′-5, respectively, instead of Intermediate G-7 or H-7 similarly to the methods of Example 661.

### Example 673 and Example 674

### Synthesis of (R)-5-cyclopentyl-4-ethyl-7-(2-(trifluoromethyl)-1H-imidazol-1-yl)-4,5-dihydro-[1,2,4]triazolo[4,3-f]pteridine (673) and (R)-5-cyclopentyl-4-ethyl-1-methyl-7-(2-(trifluoromethyl)-1H-imidazol-1-yl)-4,5-dihydro-[1,2,4]triazolo[4,3-f]pteridine (674)

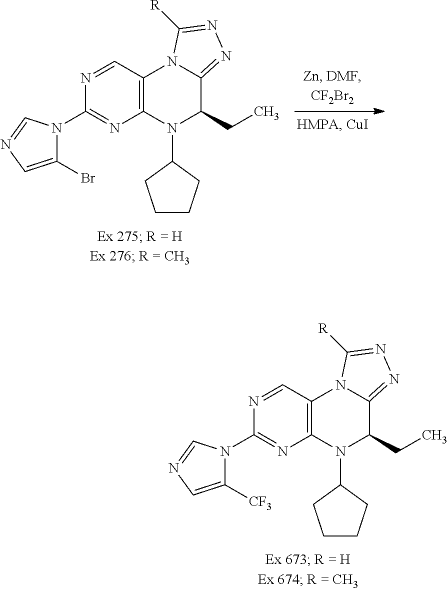

Through a suspension of activated zinc (9.868 mmol) in DMF, CF₂Br₂ is bubbled for 5 minutes. A color change to dark red occurs and the reaction mixture is stirred at rt for 2 h. The temperature is decreased to 0° C. and HMPA is added, followed by CuI (1.85 mmol) and Example 275 or Example 276 (0.616 mmol). The reaction mixture is warmed to rt, and then is plunged into a preheated 50° C. oil bath and is stirred for 18 h. The reaction mixture is cooled to rt and concentrated. The resulting residue is dissolved in DCM and is washed with water, dried with Na₂SO₄, filtered and concentrated. The resulting residue is purified by reverse phase HPLC to provide the title compounds.

### Example 675 and Example 676

### Synthesis of (R)-5-(4-ethyl-5-isopropyl-4,5-dihydro-[1,2,4]triazolo[4,3-f]pteridin-7-yl)-4-(4-fluorophenyl)thiazole-2-carbonitrile (675) and (R)-5-(4-ethyl-5-isopropyl-1-methyl-4,5-dihydro-[1,2,4]triazolo[4,3-f]pteridin-7-yl)-4-(4-fluorophenyl)thiazole-2-carbonitrile (676)

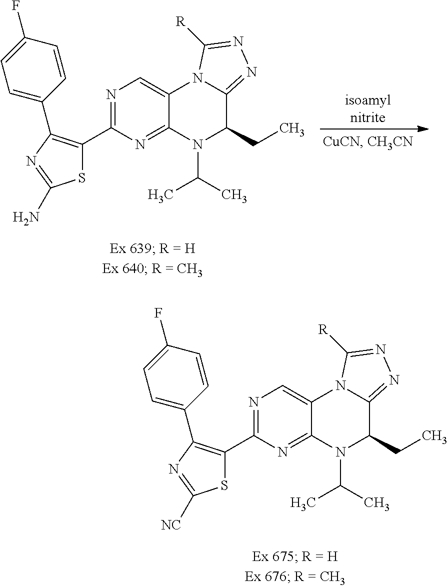

To a solution of Example 639 or Example 640 (0.586 mmol) and copper cyanide (0.598 mmol) in anhydrous acetonitrile, isoamyl nitrite (0.751 mmol) is added. The reaction is placed in an oil bath set at 90° C. under condenser with N₂ (g) inlet. The reaction mixture is stirred for 1 h, then cooled and quenched with water and extracted with EtOAc. The organic phase is collected, dried with sodium sulfate, filtered and concentrated. The resulting material is purified by preparative HPLC to give the title compounds.

### Example 679 and Example 680

### Synthesis of (R)-4-ethyl-7-(2-(4-fluorophenyl)-1H-imidazol-1-yl)-5-(1-methyl-1H-pyrazol-4-yl)-4,5-dihydro-[1,2,4]triazolo[4,3-f]pteridine, (S)-4-ethyl-7-(2-(4-fluorophenyl)-1H-imidazol-1-yl)-5-(1-methyl-1H-pyrazol-4-yl)-4,5-dihydro-[1,2,4]triazolo[4,3-f]pteridine (679) (R)-4-ethyl-7-(2-(4-fluorophenyl)-1H-imidazol-1-yl)-1-methyl-5-(1-methyl-1H-pyrazol-4-yl)-4,5-dihydro-[1,2,4]triazolo[4,3-f]pteridine, (S)-4-ethyl-7-(2-(4-fluorophenyl)-1H-imidazol-1-yl)-1-methyl-5-(1-methyl-1H-pyrazol-4-yl)-4,5-dihydro-[1,2,4]triazolo[4,3-f]pteridine (680)

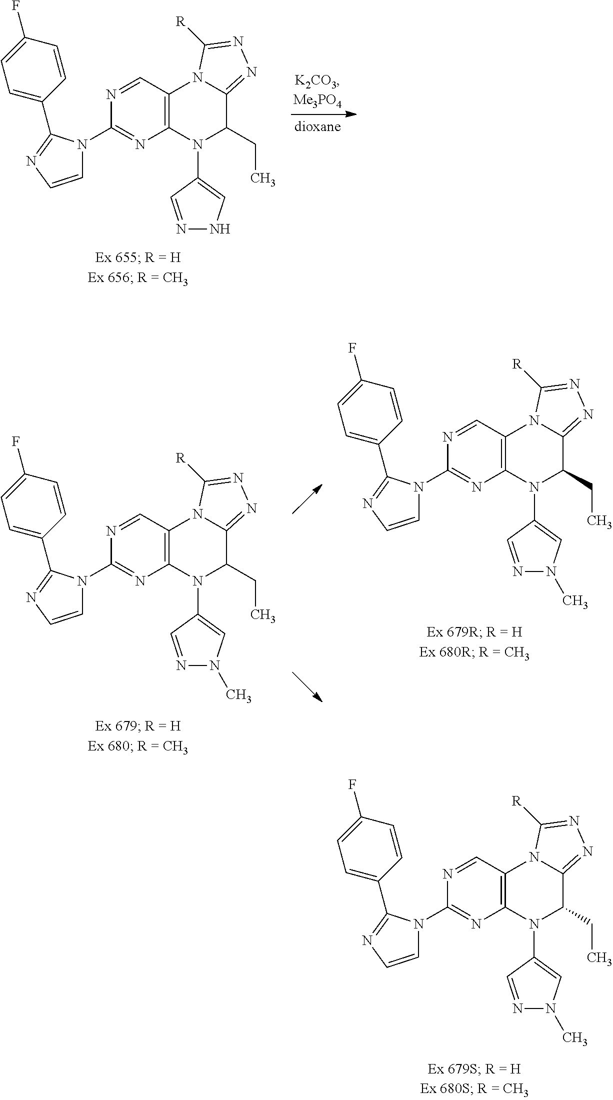

Example 655 or Example 656 (0.131 mmol) is dissolved in dioxane and Me₃PO₄ (0.262 mmol) and K₂CO₃ (0.655 mmol) are added and the reaction mixture is stirred for 18 h at 90° C. The reaction mixture is diluted with brine and extracted with EtOAc. The organic phase is dried with Na₂SO₄, filtered, concentrated under vacuum and purified by HPLC to give the title compounds. The resulting racemic mixture is resolved by chiral HPLC using an isocratic mixture of Ethanol: Hexane (33:67, 1 mL/min) eluting from a ChiralPak IA (5×50 cm) column to give isolated isomers of Example 679 and Example 680.

Additional compounds are prepared similarly to this method and the methods of Examples 541 and 655, optionally replacing Intermediate FF with a suitable intermediate and/or 2-(3,4-difluorophenyl)-1H-imidazole with a suitable imidazole in the method of Example 541, then deprotected similarly to Example 655/6 and methylated similarly to Example 679/80. In some instances, where a racemic mixture results, the two enantiomers may be isolated by chiral chromatography. The following compounds are prepared:
(R)-4-ethyl-5-(1-methyl-1-pyrazol-4-yl)-7-(2-phenyl-1H-imidazol-1-yl)-4,5-dihydro-[1,2,4]triazolo[4,3-f]pteridine and (S)-4-ethyl-5-(1-methyl-1H-pyrazol-4-yl)-7-(2-phenyl-1H-imidazol-1-yl)-4,5-dihydro-[1,2,4]triazolo[4,3-f]pteridine (Example 739),
(R)-4-ethyl-1-methyl-5-(1-methyl-1H-pyrazol-4-yl)-7-(2-phenyl-1H-imidazol-1-yl)-4,5-dihydro-[1,2,4]triazolo[4,3-f]pteridine and (S)-4-ethyl-1-methyl-5-(1-methyl-1H-pyrazol-4-yl)-7-(2-phenyl-1H-imidazol-1-yl)-4,5-dihydro-[1,2,4]triazolo[4,3-f]pteridine (Example 740),
4-ethyl-5-(1-methyl-1H-pyrazol-3-yl)-7-(2-phenyl-1H-imidazol-1-yl)-4,5-dihydro-[1,2,4]triazolo[4,3-f]pteridine (Example 741),
4-ethyl-1-methyl-5-(1-methyl-1H-pyrazol-3-yl)-7-(2-phenyl-1H-imidazol-1-yl)-4,5-dihydro-[1,2,4]triazolo[4,3-f]pteridine (Example 742),
7-(2-(1H-pyrazol-5-yl)-1H-imidazol-1-yl)-4-ethyl-5-(1-methyl-1H-pyrazol-4-yl)-4,5-dihydro-[1,2,4]triazolo[4,3-f]pteridine (Example 743), and
7-(2-(1H-pyrazol-5-yl)-1H-imidazol-1-yl)-4-ethyl-1-methyl-5-(1-methyl-1H-pyrazol-4-yl)-4,5-dihydro-[1,2,4]triazolo[4,3-f]pteridine (Example 744).
   The following table provides the example number (column 1), Intermediate (column 2), and imidazole reactant (column 3) used to give the compound shown in column 4.

| Ex. | | Imidazole | |
|---|---|---|---|
| No. | Int. | reactant | Compound structure |
| 739(R) | KK | 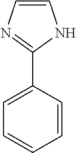 | 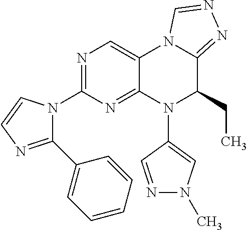 |
| 739(S) | | | 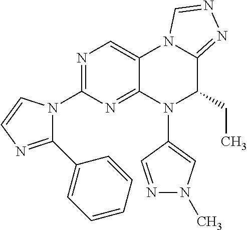 |
| 740(R) | KK′ | 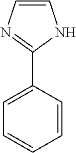 | 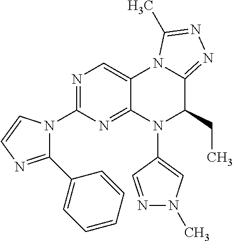 |
| 740(S) | | | 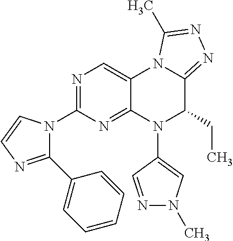 |
| 741 | QQ | 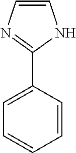 | 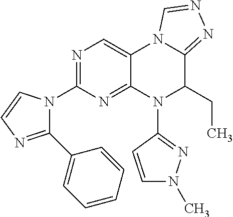 |
| 742 | QQ′ | | 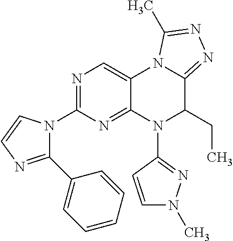 |
| 743 | KK | 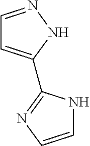 | 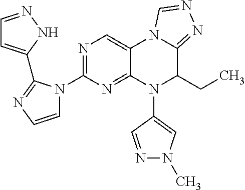 |
| 744 | KK′ | 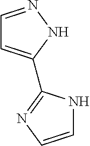 | 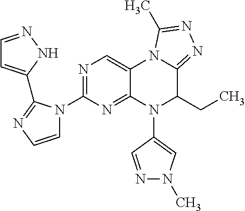 |

### Example 681 and Example 682

### Synthesis of (R)-13a-ethyl-7-(4-phenyl-1,2,3-thiadiazol-5-yl)-10,11,13,13a-tetrahydro-[1,4]oxazino[3,4-h][1,2,4]triazolo[4,3-f]pteridine, (S)-13a-ethyl-7-(4-phenyl-1,2,3-thiadiazol-5-yl)-10,11,13,13a-tetrahydro-[1,4]oxazino[3,4-h][1,2,4]triazolo[4,3-f]pteridine (681) (R)-13a-ethyl-3-methyl-7-(4-phenyl-1,2,3-thiadiazol-5-yl)-10,11,13,13a-tetrahydro-[1,4]oxazino[3,4-h][1,2,4]triazolo[4,3-f]pteridine, (S)-13a-ethyl-3-methyl-7-(4-phenyl-1,2,3-thiadiazol-5-yl)-10,11,13,13a-tetrahydro-[1,4]oxazino[3,4-h][1,2,4]triazolo[4,3-f]pteridine (682)

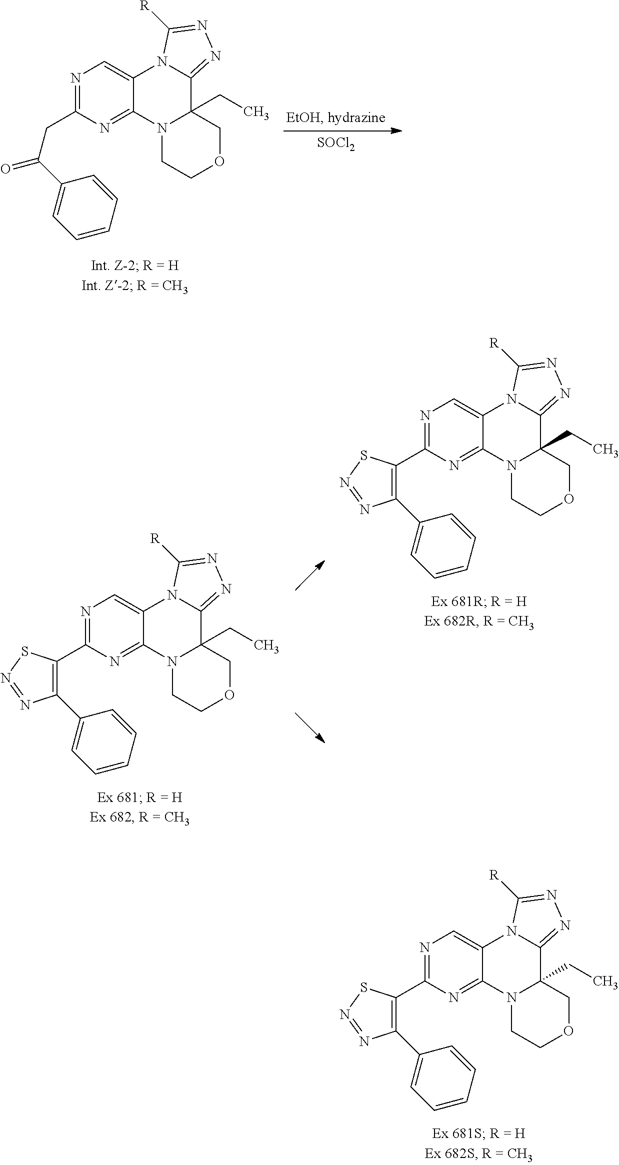

To a solution of Intermediate Z-2 or Z′-2 (0.2109 mmol) in EtOH, hydrazine (0.707 mmol) is added. The reaction mixture is plunged into a preheated 80° C. oil bath and is stirred for 18 h. The reaction mixture is cooled to rt and concentrated. Thionyl chloride is slowly added to the resulting residue. The reaction mixture is stirred for 15 minutes, then concentrated. The resulting residue is dissolved in DCM and washed with saturated NaHCO₃, dried with Na₂SO₄, filtered and concentrated to give a racemic mixture of the two title compounds. The resulting racemic mixture is resolved by chiral HPLC using an isocratic mixture of EtOH:hexane (20:80; 1 mL/min) as eluent with a Chiracel IA 4.6×250 mm column to give isolated isomers of Example 681 and Example 682.
(R)-7-(4-(2,4-difluorophenyl)-1,2,3-thiadiazol-5-yl)-13a-ethyl-10,11,13,13a-tetrahydro-[1,4]oxazino[3,4-h][1,2,4]triazolo[4,3-f]pteridine, (S)-7-(4-(2,4-difluorophenyl)-1,2,3-thiadiazol-5-yl)-13a-ethyl-10,11,13,13a-tetrahydro-[1,4]oxazino[3,4-h][1,2,4]triazolo[4,3-f]pteridine (Example 691),
(R)-7-(4-(2,4-difluorophenyl)-1,2,3-thiadiazol-5-yl)-13a-ethyl-3-methyl-10,11,13,13a-tetrahydro-[1,4]oxazino[3,4-h][1,2,4]triazolo[4,3-f]pteridine, (S)-7-(4-(2,4-difluorophenyl)-1,2,3-thiadiazol-5-yl)-13a-ethyl-3-methyl-10,11,13,13a-tetrahydro-[1,4]oxazino[3,4-h][1,2,4]triazolo[4,3-f]pteridine (Example 692),
(R)-13a-ethyl-7-(4-(5-fluoropyridin-2-yl)-1,2,3-thiadiazol-5-yl)-10,11,13,13a-tetrahydro-[1,4]oxazino[3,4-h][1,2,4]triazolo[4,3-f]pteridine and (S)-13a-ethyl-7-(4-(5-fluoropyridin-2-yl)-1,2,3-thiadiazol-5-yl)-10,11,13,13a-tetrahydro-[1,4]oxazino[3,4-h][1,2,4]triazolo[4,3-f]pteridine (Example 747), and
(R)-13a-ethyl-7-(4-(5-fluoropyridin-2-yl)-1,2,3-thiadiazol-5-yl)-3-methyl-10,11,13,13a-tetrahydro-[1,4]oxazino[3,4-h][1,2,4]triazolo[4,3-f]pteridine and (S)-13a-ethyl-7-(4-(5-fluoropyridin-2-yl)-1,2,3-thiadiazol-5-yl)-3-methyl-10,11,13,13a-tetrahydro-[1,4]oxazino[3,4-h][1,2,4]triazolo[4,3-f]pteridine (Example 748),

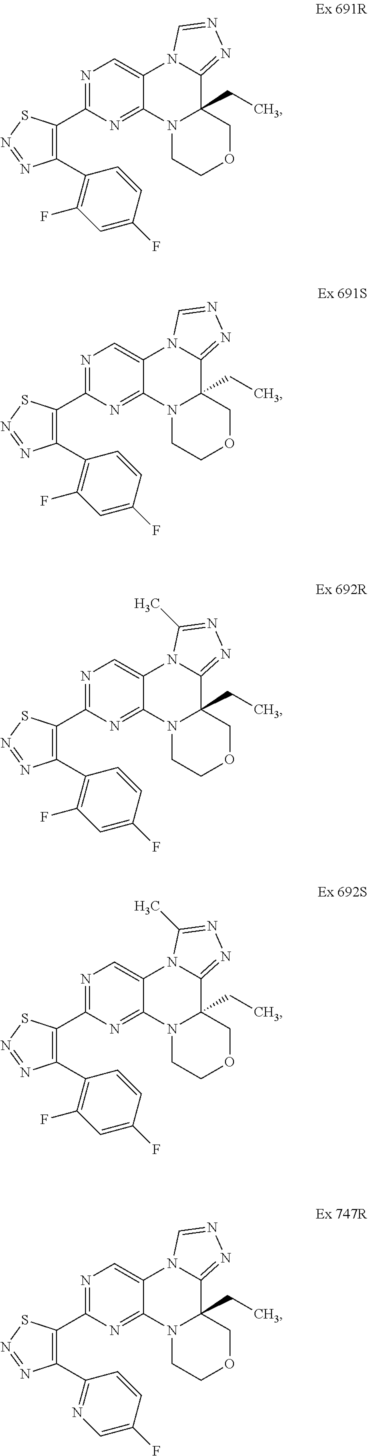

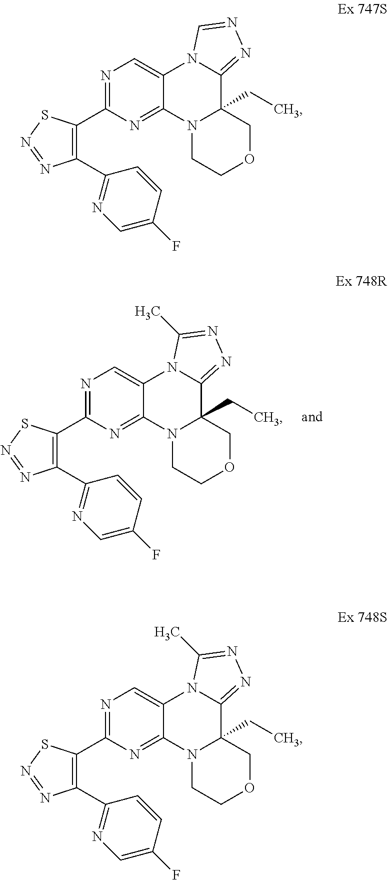

are prepared similarly, with Intermediate Z-4, Z′-4, Z-5, or Z′-5, respectively, instead of Intermediate Z-2 or Z′-2, and can be resolved, for example, using an isocratic mixture of EtOH:Hexane (3:7, 1 mL/min) as eluent from a ChiralPak IC column to give the isolated isomers of Example 691 and Example 692 and Example 747 and Example 748.

### Example 683 and Example 684

### Synthesis of (S)-13a-ethyl-12-methyl-7-(2-phenyl-1H-imidazol-1-yl)-11,12,13,13a-tetrahydro-10H-pyrazino[2,1-h][1,2,4]triazolo[4,3-f]pteridine, (R)-13a-ethyl-12-methyl-7-(2-phenyl-1H-imidazol-1-yl)-11,12,13,13a-tetrahydro-10H-pyrazino[2,1-h][1,2,4]triazolo[4,3-f]pteridine (683) and (S)-13a-ethyl-3,12-dimethyl-7-(2-phenyl-1H-imidazol-1-yl)-11,12,13,13a-tetrahydro-10H-pyrazino[2,1-h][1,2,4]triazolo[4,3-f]pteridine, (R)-13a-ethyl-3,12-dimethyl-7-(2-phenyl-1H-imidazol-1-yl)-11,12,13,13a-tetrahydro-10H-pyrazino[2,1-h][1,2,4]triazolo[4,3-f]pteridine (684)

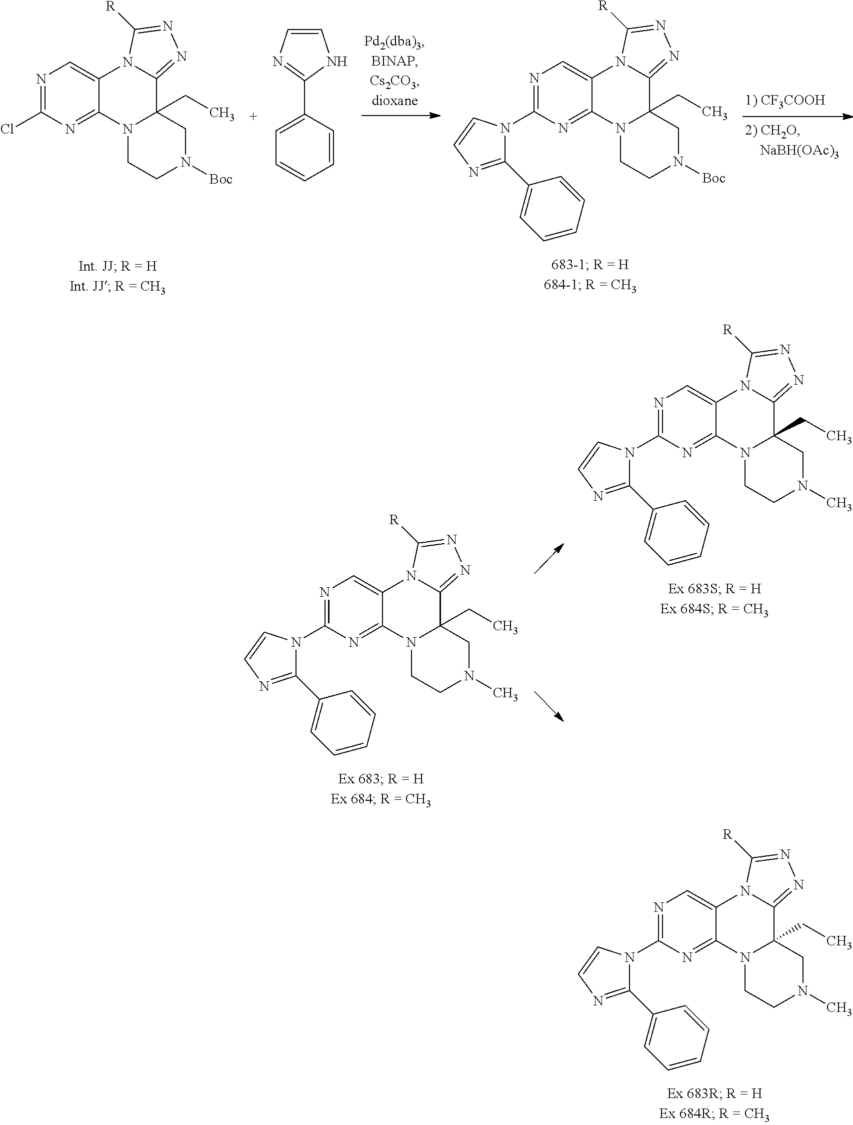

Compound 683-1 or 684-1 is prepared similarly to the methods described in Example 487/488 with Intermediate JJ or JJ′ instead of Intermediate G or H and with 2-phenyl-1H-imidazole instead of 2-(3,5-dichlorophenyl)-1H-imidazole.

Compound 683-1 or 683-2 (0.22 mmol) is dissolved in dry DCM at 0° C., and trifluoroacetic acid is added. This is then allowed to warm to rt for 2 h, then concentrated and dissolved in 1,2-dichloroethane and formalin (37% in water) and sodium triacetoxyborohydride are added with vigorous stirring at rt. After 3 h, the reaction mixture is filtered (filter cake washed with DCM), and filtrates concentrated under reduced pressure. The residue is purified by HPLC using a Phenomenex C18, 2×25 cm column with 5 μm packing, 30-70% CH₃CN/H₂O elution with 0.1% NH₄OH modifier to give the title compounds. The resulting racemic mixture can be resolved by chiral HPLC to give the isolated R and S isomers of Example 683 and Example 684.

### Example 685 and Example 686

### Synthesis of 13a-ethyl-7-(4-phenyl-1H-1,2,3-triazol-5-yl)-10,11,13,13a-tetrahydro-[1,4] oxazino[3,4-h][1,2,4]triazolo[4,3-f]pteridine (685) and 13a-ethyl-3-methyl-7-(4-phenyl-1H-1,2,3-triazol-5-yl)-10,11,13,13a-tetrahydro-[1,4]oxazino[3,4-h][1,2,4]triazolo[4,3-f]pteridine (686)

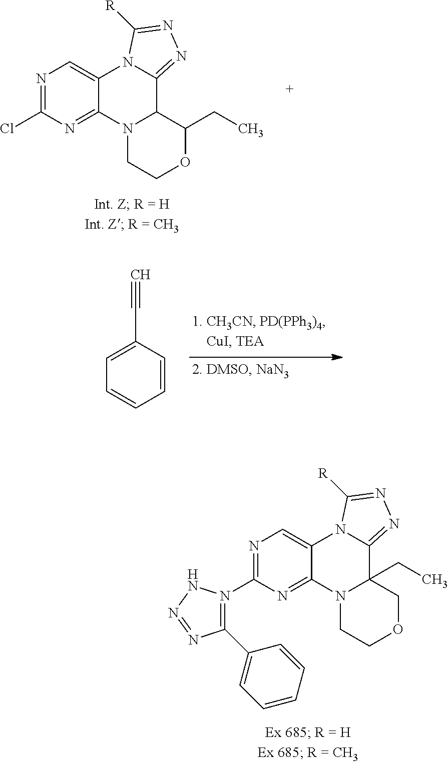

To a solution of Intermediate Z or Z′ (0.247 mmol) in acetonitrile, Pd(PPh₃)₄ (0.007 mmol), phenylacetylene (0.296 mmol), CuI (0.007 mmol), and triethylamine (0.741 mmol) are added. The reaction mixture is microwaved for 25 minutes at 140° C. The reaction mixture is filtered and concentrated. The resulting residue is purified by flash chromatography (30% EtOAc in hexanes). The resulting residue is dissolved in DMSO and sodium azide (0.071 mmol) is added. The reaction mixture is microwaved for 30 minutes at 175° C. The reaction mixture is diluted with EtOAc, washed with water, dried with Na₂SO₄, filtered and concentrated. The resulting residue is purified by reverse phase HPLC to provide the title compounds.

### Example 693 and Example 694

### Synthesis of (R)-5-(5-cyclopentyl-4-ethyl-4,5-dihydro-[1,2,4]triazolo[4,3-f]pteridin-7-yl)thiazole (693) and (R)-5-(5-cyclopentyl-4-ethyl-1-methyl-4,5-dihydro-[1,2,4]triazolo[4,3-f]pteridin-7-yl)thiazole (694)

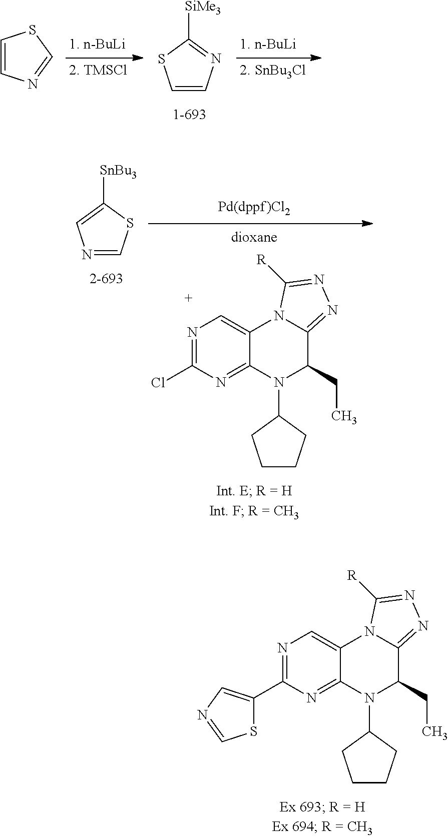

To a mixture of n-BuLi (2.5 M in hexane, 24 mL) and 18 mL of ether, a solution of 5.03 g thiazole dissolved in 59 mL of ether was added dropwise at −78° C. After 30 min, TMSCl (6.41 g) dissolved in 59 mL of ether was added at −78° C. The reaction mixture was stirred at −78° C. for 1 h and allowed to warm up to rt. The mixture was washed with saturated NaHCO₃ solution, dried over Na₂SO₄ and the solvent was evaporated. The residue was distilled (80° C./14 mmHg) to yield the desired compound 1-355 (yield: 90%); GC-MS: 157.10 m/z (M+H)⁺.

n-BuLi (2.5 M in hexane, 7.88 mmol) was added to a solution of 1-355 (826 mg, 5.25 mmol) in 45 mL of anhydrous ether and stirred at −78° C. under Ar. After 20 min, tri-n-butylstannyl chloride (2.57 g, 7.88 mmol) was added, the solution was allowed to warm to room temperature, and stirred for another 1 h. The mixture was quenched and washed with 1N sodium hydroxide, dried with MgSO₄, and the solvent was evaporated to give compound 2-355. (2 g, 100%); LCMS (0.05% TFA): 376.1 m/z (M+H)⁺.

Compound 2-355 (5 eq) and Intermediate E or Intermediate F (1 eq) are dissolved in dry 1,4-dioxane; Pd(dppf)Cl₂ (0.1 eq) is added and the resulting solution is stirred at 100° C. for 16 h. This is diluted with EtOAc and washed with water and brine, and purified by silica gel column to give the title compounds.

### Example 695

### Synthesis of (R)-2-(4-(5-cyclobutyl-4-ethyl-4,5-dihydro-[1,2,4]triazolo[4,3-f]pteridin-7-yl)-1H-pyrazol-3-yl)thiazole (695)

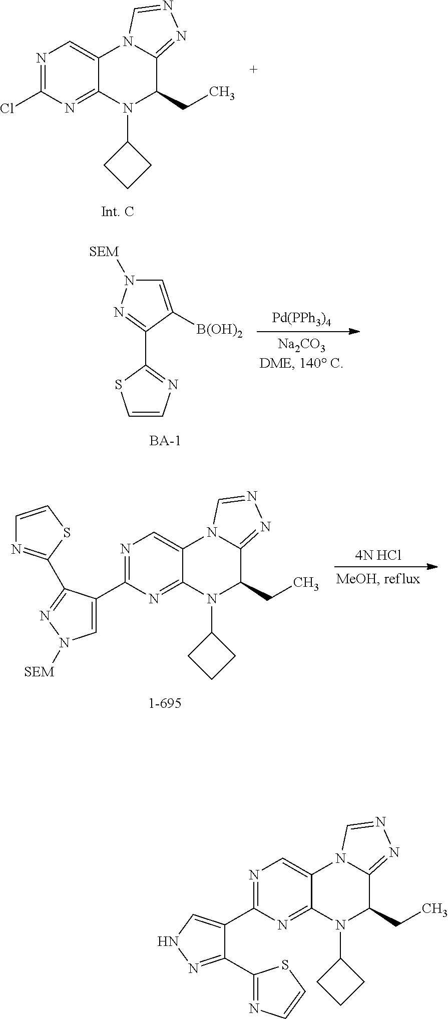

To a solution of (R)-7-chloro-5-cyclobutyl-4-ethyl-4,5-dihydro-[1,2,4]triazolo[4,3-f]pteridine (Intermediate C, 183 mg, 0.6295 mmol) in 1.8 mL of DME, Pd(PPh₃)₄ (220 mg, 0.189 mmol), Na₂CO₃ (0.95 mL, 1.89 mmol) and 3-(thiazol-2-yl)-1-42-(trimethylsilyl)ethoxy)methyl)-1H-pyrazol-4-ylboronic acid (BA-1, 205 mmol, 0.6295 mmol) were added. The reaction mixture was heated under microwave condition at 140° C. for 1 h. The reaction mixture was subjected directly to silica gel chromatography to give compound 1-695. LCMS: 536.2 m/z (M+H)⁺.

To a stirring mixture of (R)-2-(4-(5-cyclobutyl-4-ethyl-4,5-dihydro-[1,2,4]triazolo[4,3-f]pteridin-7-yl)-1-(2-(trimethylsilyl)ethoxy)methyl)-1H-pyrazol-3-yl)thiazole (compound 1-695) in 5 mL of MeOH, 10 mL of 4 N HCl in dioxane was added dropwise. The resulting mixture was warmed to reflux until all the starting material was consumed. The reaction mixture was cooled to rt and concentrated, then further purified by preparative HPLC to give the title compound. LCMS: 406.1 m/z (M+H)⁺; ¹H-NMR (CDCl₃, 300 MHz): δ 9.22 (s, 1H), 9.0 (s, 1H), 8.85 (s, 1H), 8.12-8.11 (m, 1H), 7.88-7.87 (m, 1H), 5.59-5.55 (m, 1H), 4.82-4.77 (m, 1H), 2.75-1.91 (m, 8H), 0.87 (t, J=7.4 Hz, 3H).

(R)-2-(4-(5-cyclobutyl-4-ethyl-1-methyl-4,5-dihydro-[1,2,4]triazolo[4,3-f]pteridin-7-yl)-1H-pyrazol-3-yl)thiazole (Example 696)

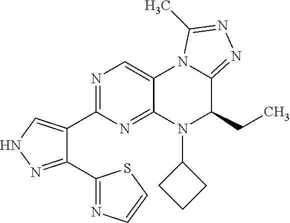

is prepared similarly, with Intermediate D instead of Intermediate C.

### Example 697 and Example 698

### Synthesis of 7-ethyl-5-methyl-8-(1-methyl-1H-pyrazol-4-yl)-2-(5-(pyridin-2-yl)-1H-pyrazol-4-yl)-7,8-dihydropteridin-6(5H)-one

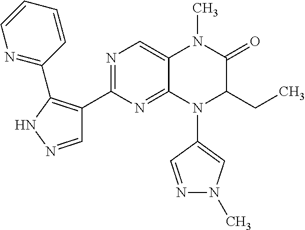

The title compound was prepared similarly to the methods described in Example 5, with Intermediate KK-3 instead of Intermediate B and with 5-(pyridin-2-yl)-1-((2-(trimethylsilyl)ethoxy)methyl)-1H-pyrazol-4-ylboronic acid (Boronic Acid 3) instead of pyridin-4-ylboronic acid. The resulting coupling product is then deprotected by the method described in Example 331 to give the title compound. LCMS: 463.1 m/z (M+H)⁺; ret. Time: 4.16 min. (Analytical Method A).

### Example 735-738

### Synthesis of 4-ethyl-7-(2-(4-fluorophenyl)-1H-imidazol-1-yl)-5-(1-methyl-1H-pyrazol-3-yl)-4,5-dihydro-[1,2,4]triazolo[4,3-f]pteridine (735), 4-ethyl-7-(2-(4-fluorophenyl)-1H-imidazol-1-yl)-1-methyl-5-(1-methyl-1H-pyrazol-3-yl)-4,5-dihydro-[1,2,4]triazolo[4,3-f]pteridine (736), 4-ethyl-7-(2-(4-fluorophenyl)-1H-imidazol-1-yl)-5-(1-methyl-1H-pyrazol-5-yl)-4,5-dihydro-[1,2,4]triazolo[4,3-f]pteridine (737), and 4-ethyl-7-(2-(4-fluorophenyl)-1H-imidazol-1-yl)-1-methyl-5-(1-methyl-1H-pyrazol-5-yl)-4,5-dihydro-[1,2,4]triazolo[4,3-f]pteridine (738)

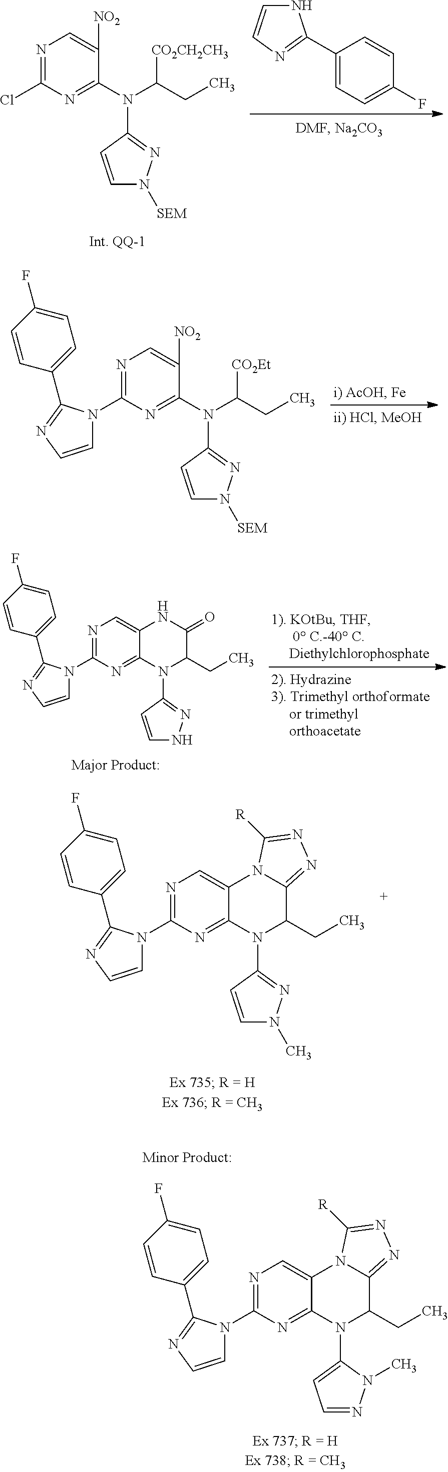

The title compounds are prepared similarly to the methods described herein, for example, the first step is similar to that of Example 13, with Intermediate QQ-1 instead of Intermediate A, and 2-(2-fluorphenyl)-1H-imidazole instead of 1H-imidazole, the next step is similar to the analogous step in the preparation of Intermediate B, with an additional deprotection step as described in Example 655. The deprotected intermediate is then reacted similarly to the last step of Example 3, resulting in a mixture of compounds with methylation at either nitrogen of the pyrazole ring.

### Example A

### In Vitro Kinase Activities (PLK TR-FRET Peptide Assay)

Compounds as described herein (compounds of Formula I, e.g., compounds of the above Examples) are tested for their in vitro kinase activities using various PLK assays. An exemplary assay procedure is described below.

(1) Test compound solution preparation: prepare 4× compound solution in PLK assay buffer (50 mM HEPES, 10 mM MgCl₂, 1 mM EGTA, 0.01% Tween-20, pH7.4). DTT is added to the buffer just before the experiment to a final concentration of 2 mM. Add 2.5 μl/well to a black 384-well low volume plate (4% DMSO at this step).
(2) Kinase preparation: Prepare 2×GST-PLK 1, 2 or 3 (e.g., CarnaBio) solutions in assay buffer (6 nM for PLK1, 6 nM for PLK2 and 0.2 nM for PLK3). Add 5 μl/well, shake the plate, incubate the enzyme with compound at ret. Time for 15 min.
(3) ATP/substrate mixture preparation: Prepare 4× (ATP/ULight-Topo Ha peptide substrate; e.g., Perkin Elmer) mixture in assay buffer (0.4 mM ATP/200 nM peptide). Add 2.5 μl/well, shake the plate and incubate at ret. Time. Reactions time: 60 min for PLK1, 60 min for PLK2 and 15 min for PLK3
(4) EDTA preparation: Dilute 0.5M EDTA to 24 mM with detection buffer. Add 5 μl/well to the plate, shake the plate well for 5 min.
(5) Prepare 4× Eu-anti-P-Topo Ha (T1342) (e.g., Perkin Elmer) solution (8 nM) in detection buffer (50 Tris-HCl, 150 mM NaCl, 0.5% BSA, PH7.5). Add 5 μl/well to the plate, shake the plate and incubate at ret. Time for 1 h before reading on Envision at 665 nm/615 nm. The fluorescent signal as a function of compound concentration is used to determine the compound IC₅₀.

The following table summarizes exemplary compounds from the Examples above and their in vitro IC₅₀ values as determined using the procedures of Example A. For IC₅₀ values in the table, (+++) indicates IC₅₀<1 μM, (++) indicates IC₅₀ of 1-10 μM, (+) indicates 10 μM<IC₅₀<50 μM, and (−) indicates IC₅₀>50 μM. For PLK2/PLK1 selectivity, (+++) indicates a ratio of IC₅₀(PLK2)/IC₅₀(PLK1) of <0.02, (++) indicates a ratio of IC₅₀(PLK2)/IC₅₀(PLK1) of 0.02 to 0.1, (+) indicates a ratio of IC₅₀(PLK2)/IC₅₀(PLK1) of 0.1 to 0.5, and (−) indicates a ratio of IC₅₀(PLK2)/IC₅₀(PLK1) of >0.5.

| Example | Plk2 IC₅₀ | Plk1 IC₅₀ | Plk3 IC₅₀ | Plk2 IC₅₀ (μM) |
|---|---|---|---|---|
| No. | (μM) | (μM) | (μM) | Plk1 IC₅₀ (μM) |
| 1 | (+++) | (+) | (+) | (+++) |
| 3 | (+++) | (+++) | (++) | (+++) |
| 5 | (+++) | (+++) | (++) | (+++) |
| 7 | (+++) | (++) | (+) | (+++) |
| 9 | (+++) | (+++) | (++) | (+++) |
| 11 | (+++) | (++) | (+) | (+++) |
| 695 | (+++) | (++) | (++) | (++) |

The compounds as described herein can be readily prepared from compounds as described in PCT International Publication Number WO 2011/079118, which describes compounds of the following Formula:

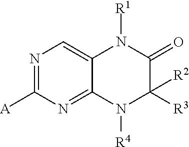

wherein the variables R², R³, R⁴ and A are similar to the analogous variables in compounds as described herein (e.g. compounds of Formula I). Intermediates wherein R¹ is H may be converted to compounds as described herein, e.g. by following the methods of Scheme 1 in converting Compound D to Compound E or E′. Compounds of Example 1, 3, 5 and 7, when compared to analogous compounds of WO 2011/079118 (i.e. compounds where R², R³, R⁴ and A are the same, and the only difference is R¹ as methyl vs. compounds as described herein with a fused ring) have comparable activity with respect to PLK2, with comparable selectivity relative to PLK1, as shown by their in vitro IC₅₀ values in the following table:

| Compound | Structure | Plk2 IC₅₀ (μM) | Plk1 IC₅₀ (μM) | $\frac{Plk2 {IC}_{50} ( μM )}{Plk1 {IC}_{50} ( μM )}$ |
|---|---|---|---|---|
| Example 1 | 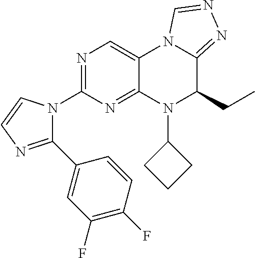 | 0.092 | 14.6 | 0.0063 |
| PCT Example 48 | 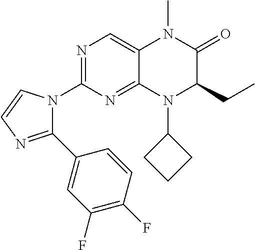 | 0.039 | 3.56 | 0.011 |
| Example 3 | 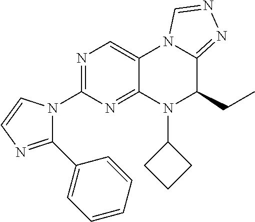 | 0.0085 | 0.441 | 0.019 |
| PCT Example 47 | 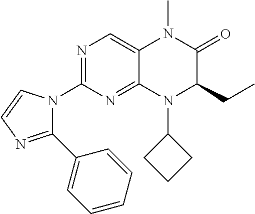 | 0.007 | 0.194 | 0.036 |
| Example 5 | 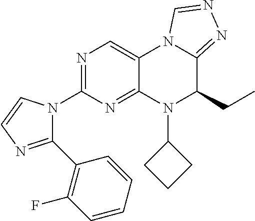 | 0.006 | 0.317 | 0.019 |
| PCT Example 49 | 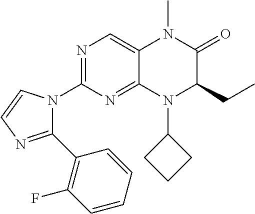 | 0.0045 | 0.077 | 0.058 |
| Example 7 | 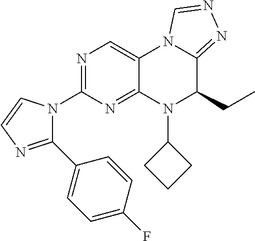 | 0.04 | 6.26 | 0.0064 |
| PCT Example 50 | 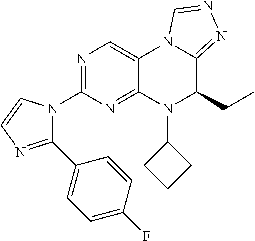 | 0.02 | 2.06 | 0.0097 |

### Example B

### Cell Activities (293-Syn/PLK2 Cell Assay)

Compounds as described herein (compounds of Formula I, e.g., compounds of the above Examples) are tested for their activity in HEK-293 cells expressing α-synuclein and PLK2. An exemplary assay procedure is described below.

(1) Plate HEK-293 cells stably transfected with α-synuclein in 10 cm dishes (Corning) at 1.5e6 cells/cm₂ in 10% FCS/DMEM.
(2) Transfect cells with PLK2 (PLK2-pCMV6 (Origene), at a concentration of 24 μg/dish and 72 μl Fugene6/dish (Roche)).
(3) Trypsinize cells the following day and plate at 30,000 cells/well in PDL coated 96 well tissue culture plates (Becton Dickinson).
(4) Starting with compounds at a concentration of 10 mM solutions, prepare five 1:3 serial dilutions in DMSO.
(5) Dilute test and positive control compound DMSO stocks 1:100 into 10% FCS DMEM. (6) Change the cell media immediately prior to compound treatment, then add compound+DMEM to cells at 1:10 final dilution (final DMSO concentration is 0.1%).
(7) After 2 hours, place the cells on ice, remove the media and the rinse cells once with cold phosphate buffered saline (PBS). Remove PBS and lyse the cells using a cell extraction buffer (CEB) (10 mM Tris, pH 7.4, 100 mM NaCl, 1 mM EDTA, 1 mM NaF, 1 mM EGTA, 20 mM Na4P2O7, 2 mM Na3VO4, 0.5% Deoxycholate, 1% TritonX-100, 10% Glycerol, 0.1% SDS) with added protease inhibitors (10 μg/ml leupeptin, 20 μg/ml aprotinin)
(8) Freeze plates on dry ice and store at −80°.

Total and p-Ser-129 α-synuclein levels can be quantified with a sandwich ELISA (e.g., using 1H7 as the capture antibody and biotinylated 5C12 and 11A5 as the total and phospho synuclein reporter antibodies respectively; see e.g., J. Biol. Chem. 2006, 281:29739-29752, the disclosure of which is incorporated herein in its entirety). Alpha-synuclein phosphorylated at serine 129 (p-Ser-129 α-synuclein) levels are normalized to the total synuclein measured in each lysate and the ratio of phosphorylated synuclein to total synuclein as a function of compound concentration can be used to determine IC₅₀ of the compounds. Cell activities of the compounds in Example A were all less than 5 μM, and Examples 1, 3, 5 and 7 compared to the analogous compounds of PCT International Publication Number WO 2011/079118 showed comparable cell activity, as shown by their cellular IC₅₀ values in the following table:

| | | Cell activity |
|---|---|---|
| Compound | Structure | IC₅₀ (μM) |
| Example 1 | 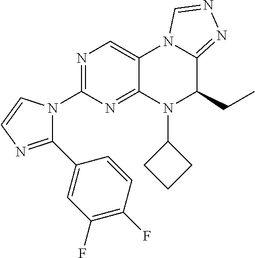 | 4.4 |
| PCT Example 48 | 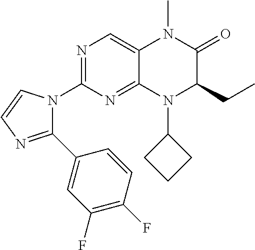 | 4.3 |
| Example 3 | 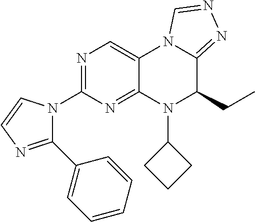 | 0.42 |
| PCT Example 47 | 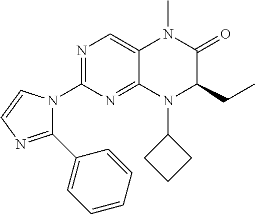 | 0.67 |
| Example 5 | 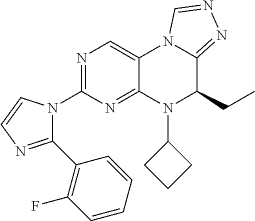 | 0.44 |
| PCT Example 49 | 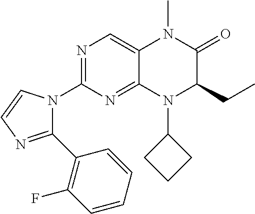 | 0.42 |
| Example 7 | 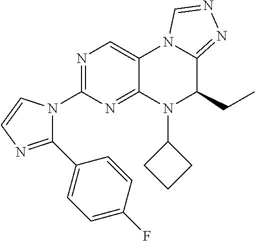 | 2.9 |
| PCT Example 50 | 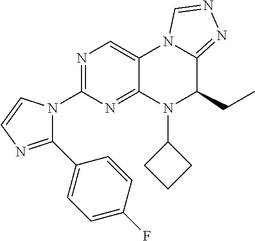 | 2.4 |

Based on comparable biochemical and cellular activity demonstrating inhibition of PLK2, it would be clear to one skilled in the art that compounds as prophetically described herein, being analogous to compounds as described in PCT International Publication Number WO 2011/079118, with the only difference being the fused triazole (or similar) ring, would have comparable PLK2 activity and selectivity compared to PLK1.

### Example C

### In Vivo Activities

Compounds as described herein (compounds of Formula I, e.g., compounds of the above Examples) can be tested for their in vivo activities, e.g., using the test procedures described in J. Biol. Chem. 2009, 284(5): 2598-2602 (see, e.g., page 2599, last paragraph), the disclosure of which is incorporated herein in its entirety. For example, mice can be dosed with the compounds of the invention at about 5 mg/kg to about 500 mg/kg (e.g., via tail vein injection) at 5 ml/kg dose volume in 0.9% saline. Mice can be euthanized (e.g., CO₂ about 3 h after dosing) and brains can be removed, rinsed in 0.9% saline and separated into left and right hemispheres. The cortex can be dissected from the right hemisphere, frozen on dry ice and stored at −80° C. until used for quantitation of alpha-synuclein levels. Tissue lysates can be prepared and analyzed, e.g., using an ELISA assay (e.g., as described in the above reference; see, e.g., page 2600, first paragraph).

Protein concentrations of lysates can be measured (e.g., using the Micro BCA Kit from Pierce Biotechnology). Total alpha-synuclein and alpha-synuclein phosphorylated at serine 129 (p-Ser-129 α-synuclein) levels can be normalized to the total protein measured in each lysate and a ratio of phosphorylated synuclein to total synuclein can be calculated. Total and p-Ser-129 α-synuclein levels can be quantified using a sandwich ELISA (e.g., using 1H7 as the capture antibody and biotinylated 11A5 as the total or phosphor synuclein reporter antibodies; see e.g., J. Biol. Chem. 2006, 281:29739-29752, the disclosure of which is incorporated herein in its entirety).

What is claimed is:

1. A compound having a structure according to Formula (I): 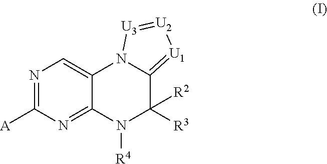
or a salt or solvate thereof, wherein:
A is a ring selected from the group consisting of substituted or unsubstituted aryl, substituted or unsubstituted 5- or 6-membered heterocycloalkyl, and substituted or unsubstituted 5- or 6-membered heteroaryl;
U¹ is N or CR¹, U² is N or CR^{1a} and U³ is N or CR^{1b}, with the proviso that any one or any two of U¹, U² and U³ is N, wherein R¹, R^{1a} and R^{1b}, if present, are independently selected from the group consisting of H, halogen, CN, unsubstituted C₁-C₄ alkyl, and C₁-C₄ haloalkyl;
R² is selected from the group consisting of H, substituted or unsubstituted C₁-C₆ alkyl, substituted or unsubstituted C₂-C₆ alkenyl, substituted or unsubstituted C₂-C₆ alkynyl, substituted or unsubstituted 3- to 6-membered heteroalkyl, substituted or unsubstituted C₃-C₆ cycloalkyl, and substituted or unsubstituted 3- to 6-membered heterocycloalkyl;
R³ is selected from the group consisting of substituted or unsubstituted C₁-C₆ alkyl, substituted or unsubstituted C₂-C₆ alkenyl, substituted or unsubstituted C₂-C₆ alkynyl, substituted or unsubstituted 3- to 6-membered heteroalkyl, substituted or unsubstituted C₃-C₆ cycloalkyl, and substituted or unsubstituted 3- to 6-membered heterocycloalkyl;
or R² and R³, together with the carbon atom to which they are attached, are optionally joined to form a substituted or unsubstituted C₃-C₆ cycloalkyl or a substituted or unsubstituted 3- to 6-membered heterocycloalkyl;
R⁴ is selected from the group consisting of substituted or unsubstituted C₁-C₁₀ alkyl, substituted or unsubstituted C₂-C₁₀ alkenyl, substituted or unsubstituted C₂-C₁₀ alkynyl, substituted or unsubstituted 3- to 10-membered heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, and —NR²⁵R²⁶; or R⁴ and R³, together with the atoms to which they are attached, are optionally joined to form a substituted or unsubstituted 3- to 8-membered heterocyclic ring; or R⁴, R² and R³, together with the atoms to which they are attached, are optionally joined to form a substituted or unsubstituted heterocyclic bicyclic ring system of fused 4- to 8-membered rings; and
R²⁵ and R²⁶ are independently H, substituted or unsubstituted C₃-C₈ cycloalkyl, or substituted or unsubstituted C₁-C₁₀ alkyl.

2. The compound of claim 1, wherein A is a member selected from pyrrolidinyl, piperidinyl, morpholinyl, thiomorpholinyl, N-alkyl-piperazinyl, oxazolidinyl, thiazolidinyl, pyridyl, pyrimidinyl, pyridazinyl, pyrazinyl, triazinyl, pyrrolyl, imidazolyl, pyrazolyl, thiazolyl, isothiazolyl, oxazolyl, isoxazolyl, thiadiazolyl, triazolyl and tetrazolyl, wherein A is substituted or unsubstituted.

3. The compound of claim 2, wherein A is a substituted or unsubstituted ring selected from the group consisting of pyridyl, pyrazolyl and imidazolyl.

4. The compound of claim 3, wherein A is a substituted or unsubstituted ring selected from the group consisting of pyridin-3-yl, pyridin-4-yl, pyrazol-4-yl and imidazol-1-yl.

5. The compound of claim 1, wherein the compound has a structure selected from the group consisting of Formula (XIIa), Formula (XIIb), Formula (XIIc), Formula (XIId), Formula (XIIe), and Formula (XIIf): 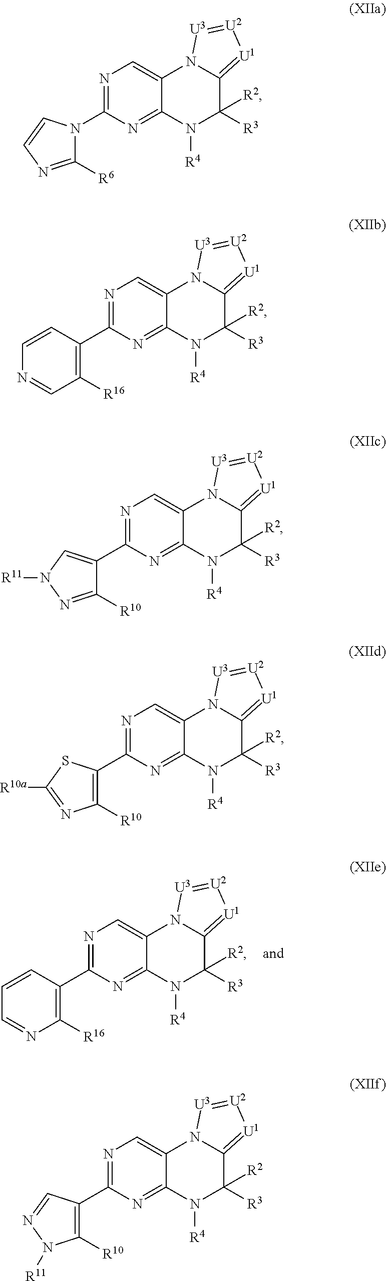
or a salt or solvate thereof, wherein:
U¹, U², U³, R², R³ and R⁴ are defined as in claim 1;
R⁶ is selected from the group consisting of H, substituted or unsubstituted alkyl, substituted or unsubstituted alkenyl, substituted or unsubstituted alkynyl, substituted or unsubstituted heteroalkyl, substituted or unsubstituted cycloalkyl, substituted or unsubstituted heterocycloalkyl, aryl optionally substituted with one or more independently selected substituents R²⁷, heteroaryl optionally substituted with one or more independently selected substituents R²⁷, —CN, -halogen, —OR¹², —SR¹², —NR¹²R¹³, —C(O)R¹⁴, —C(O)NR¹²R¹³, —OC(O)NR¹²R¹³, —C(O)OR¹², —NR¹⁵C(O)R¹⁴, —NR¹⁵C(O)OR¹², —NR¹⁵C(O)NR¹²R¹³, —NR¹⁵C(S)NR¹²R¹³, —NR¹⁵S(O)₂R¹⁴, —S(O)₂NR¹²R¹³, —S(O)R¹⁴ and —S(O)₂R¹⁴;
R¹⁰, R^{10a} and R¹⁶ are independently selected from the group consisting of H, substituted or unsubstituted alkyl, substituted or unsubstituted alkenyl, substituted or unsubstituted alkynyl, substituted or unsubstituted heteroalkyl, substituted or unsubstituted cycloalkyl, substituted or unsubstituted heterocycloalkyl, aryl optionally substituted with one or more independently selected substituents R²⁷, heteroaryl optionally substituted with one or more independently selected substituents R²⁷, —CN, -halogen, —OR²⁰, —SR²⁰, —NR²⁰R²¹, —C(O)R²², —C(O)NR²⁰R²¹, —OC(O)NR²⁰R²¹, —C(O)OR²⁰, —NR²³C(O)R²², —NR²³C(O)OR²⁰, —NR²³C(O)NR²⁰R²¹, —NR²³C(S)NR²⁰R²¹, —NR²³S(O)₂R²², —S(O)₂NR²⁰R²¹, —S(O)R²² and —S(O)₂R²²;
R¹¹ is selected from the group consisting of H, —C(O)R²², substituted or unsubstituted C₁-C₆-alkyl, substituted or unsubstituted 3- to 6-membered heteroalkyl, aryl optionally substituted with one or more independently selected substituents R²⁷, 5- or 6-membered heteroaryl optionally substituted with one or more independently selected substituents R²⁷, substituted or unsubstituted C₃-C₈ cycloalkyl and substituted or unsubstituted 3- to 8-membered heterocycloalkyl;
each occurrence of R¹², R¹³, R¹⁵, R²⁰, R²¹ and R²³ are independently selected from the group consisting of H, substituted or unsubstituted C₁-C₆ alkyl, substituted or unsubstituted 3- to 6-membered heteroalkyl, aryl optionally substituted with one or more independently selected substituents R²⁷, 5- or 6-membered heteroaryl optionally substituted with one or more independently selected substituents R²⁷, substituted or unsubstituted C₃-C₈ cycloalkyl and substituted or unsubstituted 3- to 8-membered heterocycloalkyl;
each occurrence of R¹⁴ and R²² are independently selected from the group consisting of substituted or unsubstituted C₁-C₆ alkyl, substituted or unsubstituted 3- to 6-membered heteroalkyl, aryl optionally substituted with one or more independently selected substituents R²⁷, 5- or 6-membered heteroaryl optionally substituted with one or more independently selected substituents R²⁷, substituted or unsubstituted C₃-C₈ cycloalkyl and substituted or unsubstituted 3- to 8-membered heterocycloalkyl;
R²⁷ at each occurrence is selected from the group consisting of C₁-C₁₀ alkyl optionally substituted with one or more independently selected substituents R²⁸, 3- to 10-membered heteroalkyl optionally substituted with one or more independently selected substituents R²⁸, C₃-C₈ cycloalkyl optionally substituted with one or more independently selected substituents R²⁹, 3- to 8-membered heterocycloalkyl optionally substituted with one or more independently selected substituents R²⁹, aryl optionally substituted with one or more independently selected substituents R²⁹, heteroaryl optionally substituted with one or more independently selected substituents R²⁹, —CN, —NO₂, -halogen, —OR³⁰, —SR³⁰, —NR³⁰R³¹, —C(O)R³², —C(O)NR³⁰R³¹, —OC(O)NR³⁰R³¹, —C(O)OR³⁰, —OC(O)R³², —NR³³C(O)R³², —NR³³C(O)OR³⁰, —NR³³C(O)NR³⁰R³¹, —NR³³C(S)NR³⁰R³¹, —NR³³S(O)₂R³², —S(O)₂NR³⁰R³¹, —S(O)R³² and —S(O)₂R³²;
R³⁰, R³¹, R³², and R³³, at each occurrence are independently selected from the group consisting of hydrogen, C₁-C₁₀ alkyl optionally substituted with one or more independently selected substituents R²⁸, 3- to 12-membered heteroalkyl optionally substituted with one or more independently selected substituents R²⁸, C₃-C₈ cycloalkyl optionally substituted with one or more independently selected substituents R²⁹, 3- to 8-membered heterocycloalkyl optionally substituted with one or more independently selected substituents R²⁹, aryl optionally substituted with one or more independently selected substituents R²⁹, and heteroaryl optionally substituted with one or more independently selected substituents R²⁹, provided that R³² is other than hydrogen;
R²⁸ at each occurrence is independently selected from the group consisting of aryl optionally substituted with one or more independently selected substituents R³⁹, heteroaryl optionally substituted with one or more independently selected substituents R³⁹, —OR³⁴, —SR³⁴, —NHR³⁴, —NR—C(O)R³⁴, —C(O)OR³⁴, —C(O)NHR³⁴, —C(O)NR³⁵R³⁴, —NHC(O)R³⁴, —NR³⁴C(O)R³⁴, —NHC(O)OR³⁴, —NR³⁴C(O)OR³⁴, —NR³⁴C(O)OH, —S(O)₂R³⁴, —S(O)₂NHR³⁴, —S(O)₂NR³⁵R³⁴, —NHS(O)₂R³⁴, —NR³⁴S(O)₂R³⁴, -halogen, —NHC(O)OH, —C(O)OH, —C(O)NH₂, —S(O)₂NH₂, —CN, —NO₂, ═O, —OH, ═NH, and —NH₂;
R²⁹ at each occurrence is independently —R²⁸ or —R³⁴;
R³⁴ and R³⁵ are independently selected from the group consisting of aryl optionally substituted with one or more independently selected substituents R³⁹, heteroaryl optionally substituted with one or more independently selected substituents R³⁹, and C₁-C₄ alkyl optionally substituted with one or more substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR³⁶R³⁷;
or —NR³⁴R³⁵ forms a 5-, 6-, or 7-membered heterocycloalkyl optionally substituted with one or more unsubstituted C₁-C₄ alkyl;
—NR³⁶R³⁷ forms a 5-, 6-, or 7-membered heterocycloalkyl optionally substituted with one or more unsubstituted C₁-C₄ alkyl;
R³⁹ at each occurrence is independently selected from the group consisting of —R⁴⁴, —OR⁴⁴, —SR⁴⁴, —NHR⁴⁴, —NR—C(O)R⁴⁴, —C(O)OR⁴⁴, —NHC(O)R⁴⁴, —C(O)NHR⁴⁵, —C(O)NR⁴⁴R⁴⁵, —S(O)₂R⁴⁴, —NHS(O)₂R⁴⁴, —S(O)₂NHR⁴⁵, —S(O)₂NR⁴⁴R⁴⁵, -halogen, —C(O)OH, —C(O)NH₂, —CN, —OH, and —NH₂;
R⁴⁴ and R⁴⁵ are independently C₁-C₄ alkyl optionally substituted with one or more independently selected substituents independently selected from the group consisting of —F, —OH, —NH₂, unsubstituted C₁-C₄ alkoxy, C₁-C₄ haloalkoxy, unsubstituted mono-alkylamino, unsubstituted di-alkylamino, and —NR⁴⁶R⁴⁷;
or —NR⁴⁴R⁴⁵ forms a 5-, 6-, or 7-membered heterocycloalkyl optionally substituted with one or more unsubstituted C₁-C₄ alkyl; and
—NR⁴⁶R⁴⁷ forms a 5-, 6-, or 7-membered heterocycloalkyl optionally substituted with one or more unsubstituted C₁-C₄ alkyl.

6. The compound of claim 1, wherein the compound has a structure according to Formula (XVI): 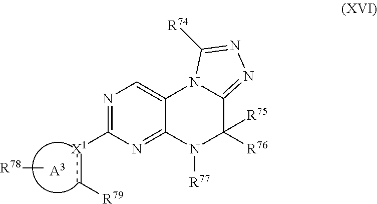
or a salt or solvate thereof, wherein:
X₁ is C or N and the dashed line represents a single or double bond;
A³ is a ring selected from the group consisting of phenyl, pyridine, pyrimidine, pyrazine, pyridazine, pyrrole, pyrazole, imidazole, thiazole, isothiazole, isoxazole, triazole, thiadiazole, benzimidazole, indole, pyrrolo[2,3-b]pyridine, quinoline, pyrrolidine, piperidine, piperazine, and dihydro-imidazole;
R⁷⁴ is hydrogen or methyl;
R⁷⁵ is hydrogen, methyl (e.g. —CD₃ or —CH₃), ethyl (e.g. —CD₂CD₃ or —CH₂CH₃), —CH₂-cyclopropyl, or —CH₂CF₃;
R⁷⁶ is methyl (e.g. —CD₃ or —CH₃), ethyl (e.g. —CD₂CD₃ or —CH₂CH₃), —CH₂-cyclopropyl, or —CH₂CF₃;
or R⁷⁵ and R⁷⁶, together with the carbon atom to which they are attached, are optionally joined to form cyclobutyl;
R⁷⁷ is selected from the group consisting of —NH₂, —NHCH₃, —NHcyclopropyl, pyrrolidine, —CH₂-cyclopropyl, —CH(CH₃)-cyclopropyl, cyclopropyl, cyclobutyl optionally substituted with 1 or 2 fluoro, cyclopentyl optionally substituted with 1 or 2 fluoro, isopropyl (e.g. —CH(CH₃)₂ or —CD(CD₃)₂), —CH₂CH₂CF₃, tetrahydropyran, tetrahydrofuran, oxetane, phenyl optionally substituted with 1 or 2 substituents R⁸⁰, pyrazole optionally substituted with 1 substituent R⁸¹, and pyrimidine;
or R⁷⁷ and R⁷⁶, together with the atoms to which they are attached, are optionally joined to form a substituted or unsubstituted 5- to 7-membered heterocyclic ring selected from the group consisting of 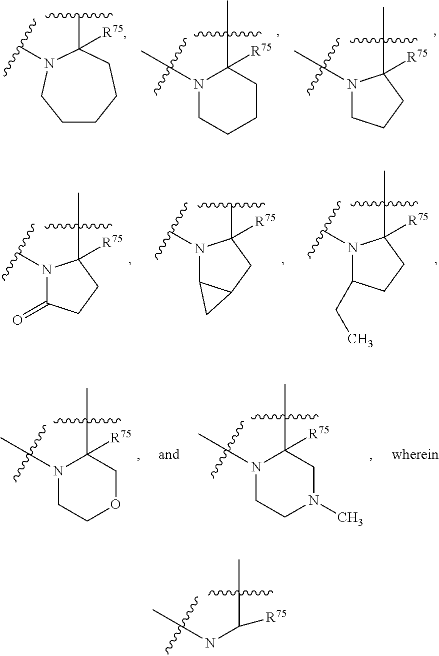
represents the core ring of Formula I, i.e. the N attached to R⁷⁷ and the C attached to R⁷⁶;
or R⁷⁷, R⁷⁵ and R⁷⁶, together with the atoms to which they are attached, are optionally joined to form a substituted or unsubstituted 7-membered heterocyclic ring selected from the group consisting of 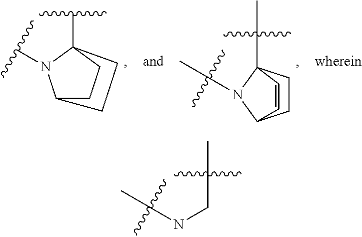
represents the core ring of Formula I, i.e. the N attached to R⁷⁷ and the C attached to R⁷⁶/R⁷⁵;
R⁷⁸ is hydrogen, —Br, —CN, —CH₃, —CH₂CN, —CH₂CH₂NH₂, —OH, —O⁻, ═O, —OCH₃, —Obenzyl, —C(O)OH, —C(O)OCH₃, —C(O)OCH₂CH₃, —C(O)NH₂, —C(O)NHCH₃, —C(O)N(CH₃)₂, 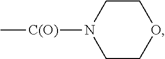
—NH₂, ═NH, —NHCH₃, —N(CH₃)₂, —NHS(O)₂CH₃, —S(O)₂CH₃, phenyl, thiazole, pyridine or pyrazine;
R⁷⁹ is hydrogen, —Cl, —Br, —CH₃, —CF₃, —CH₂NH₂, —NH₂, —CH₂NHC(O)OCH₃, —CH₂NHC(O)CH₃, —CH₂NHC(O)phenyl, —CH₂NHS(O)₂CH₃, —CH₂NHS(O)₂-phenyl, —NHC(O)CH₃, —NHC(O)OCH₃, —NHC(O)phenyl, —NHS(O)₂CH₃, —NHS(O)₂-phenyl, —CHCHphenyl, cyclopropyl, cyclopentenyl, benzyl, phenyl optionally sub with 1, 2 or 3 substituents R⁸², pyridine optionally substituted with 1 fluoro, pyrimidine, pyrazine, pyridazine, pyrazole, thiazole, oxazole, thiophene optionally substituted with 1 chloro, pyrrolidine, oxazolidinone, pyrrolidinone, dihydropyran, tetrahydropyran, morpholine, 4-methyl-piperazine, pyrrolidine-dione, pyridinone, isoquinoline, or quinoline;
R⁸⁰ at each occurrence is independently —C(O)NH₂, fluoro, chloro, cyano, pyrazole, triazole, pyridine or pyrimidine;
R⁸¹ is methyl or 2-(trimethylsilyl)ethoxy)methyl, cyclopropyl, or —CH₂-cyclopropyl; and
R⁸² at each occurrence is independently selected from the group consisting of fluoro, chloro, bromo, —S(O)₂CH₃, —OCF₃, —CF₃, —CN, pyridine, triazole, and pyrazole.

7. The compound of claim 6, wherein the compound has a structure selected from the group consisting of Formula (XVIa), Formula (XVIb), Formula (XVIc), Formula (XVId), and Formula (XVIe), 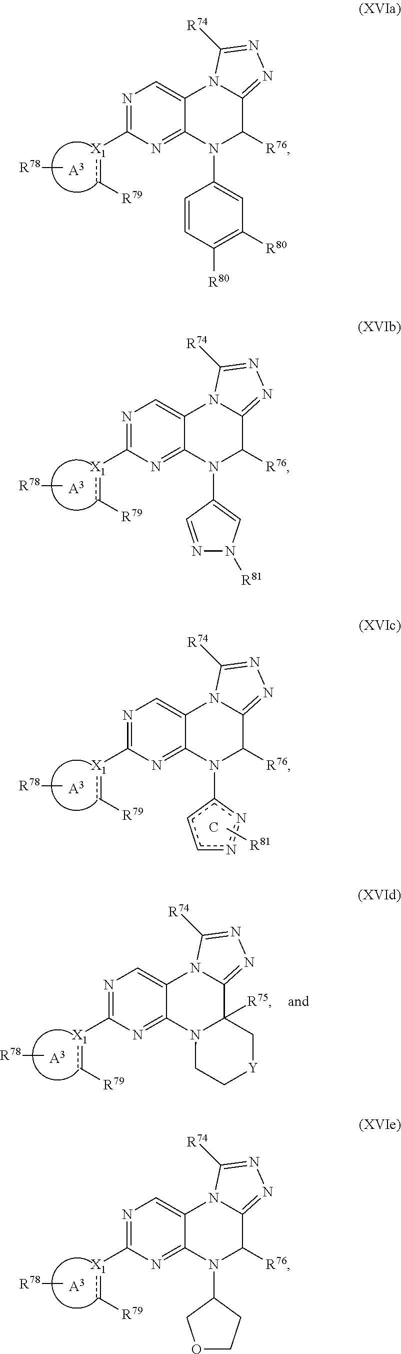
or a salt or solvate thereof, wherein:
C is pyrazole, wherein R⁸¹ is bound to either of the nitrogens in the pyrazole ring;
Y is O or N—CH₃; and
X1, A³, R⁷⁴, R⁷⁵, R⁷⁶, R⁷⁸, R⁷⁹, R⁸⁰ and R⁸¹ are as defined for claim 6.

8. The compound of claim 7, wherein the compound has a structure selected from the group consisting of Formula of Formula (XVIIa), Formula (XVIIb), Formula (XVIIc), Formula (XVIId), and Formula (XVIIe), 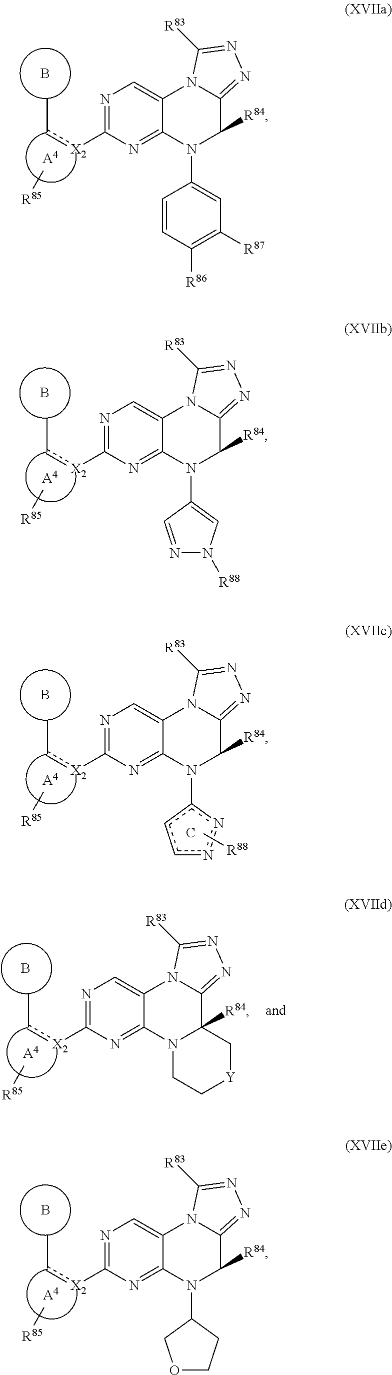
or a salt or solvate thereof, wherein:
X₂ is C or N and the dashed line represents a single or double bond;
Y is O or N—CH₃;
A⁴ is selected from the group consisting of phenyl, pyridin-2-yl, pyridin-3-yl, pyridin-4-yl, pyrimidin-5-yl, pyrazin-2-yl, pyridin-2-one, pyridin-4-imine, pyrazol-1-yl, pyrazol-4-yl, imidazol-1-yl, thiazol-5-yl, isothiazol-4-yl, isoxazol-4-yl, 1,2,3-triazol-5-yl, 1,2,4-triazol-1-yl, 1,2,3-thiadiazol-5-yl, indol-1-yl, indol-2-yl, indol-7-yl, piperazin-1-yl, 4,5-dihydro-1H-imidazol-1-yl;
B is selected from the group consisting of phenyl optionally substituted with 1, 2, or 3 substituents R⁸⁹, pyridin-2-yl, 5-fluoro-pyridin-2-yl, pyridin-3-yl, pyridin-4-yl, pyrimidin-2-yl, pyrimidin-5-yl, pyrazin-2-yl, pyridazin-3-yl, pyrazol-1-yl, pyrazol-5-yl, pyrazol-4-yl, thiazol-2-yl, thiazol-4-yl, oxazol-2-yl, pyrrolidin-1-yl, oxazolidin-2-on-3-yl, 2-oxopyrrolidin-1-yl, tetrahydro-2H-pyran-4-yl, morpholin-4-yl, 4-methyl-piperazin-1-yl, quinolin-5-yl, and quinolin-3-yl;
C is pyrazole, wherein R⁸⁸ is bound to either of the nitrogens in the pyrazole ring;
R⁸³ is hydrogen or —CH₃;
R⁸⁴ is —CD₂CD₃ or —CH₂CH₃;
R⁸⁵ is hydrogen, —CH₃, —Br, —CN, or —NH₂;
R⁸⁶ is hydrogen, —F, —Cl, —C(O)NH₂, or —CN;
R⁸⁷ is hydrogen, —F, —Cl, —C(O)NH₂, or —CN;
R⁸⁸ is hydrogen, methyl, cyclopropyl, or —CH₂-cyclopropyl; and
R⁸⁹ at each occurrence is independently selected from the group consisting of fluoro, chloro, bromo, —S(O)₂CH₃, —OCF₃, —CF₃, —CN, pyridine, triazole, and pyrazole.

9. A pharmaceutical composition comprising a compound according to claim 1 and a pharmaceutically acceptable carrier.

10. A method of treating a neurodegenerative disease comprising administering to a mammalian subject in need thereof a pharmaceutically effective amount of a compound according to claim 1 or a composition according to claim 9.

11. The method of claim 10, wherein the disease is an alpha-synucleinopathy.

12. The method of claim 11, wherein the disease is a member selected from the group consisting of Parkinson's disease, Parkinson disease with dementia, PD at risk syndrome, dementia with Lewy bodies, diffuse Lewy body disease, Lewy body dementia, cortical Lewy body disease, senile dementia of Lewy type, Lewy body variant of Alzheimer's disease, diffuse Lewy body type of Alzheimer's disease, combined Parkinson's disease and Alzheimer's disease, multiple system atrophy, striatonigral degeneration, olivopontocerebellar atrophy, and Shy-Drager syndrome.

13. The method of claim 12, wherein the disease is Parkinson's disease.

14. A pharmaceutical composition comprising a compound according to claim 6 and a pharmaceutically acceptable carrier.

15. A method of treating a neurodegenerative disease comprising administering to a mammalian subject in need thereof a pharmaceutically effective amount of a compound according to claim 6 or a composition according to claim 14.

16. The method of claim 15, wherein the disease is an alpha-synucleinopathy.

17. The method of claim 16, wherein the disease is a member selected from the group consisting of Parkinson's disease, Parkinson disease with dementia, PD at risk syndrome, dementia with Lewy bodies, diffuse Lewy body disease, Lewy body dementia, cortical Lewy body disease, senile dementia of Lewy type, Lewy body variant of Alzheimer's disease, diffuse Lewy body type of Alzheimer's disease, combined Parkinson's disease and Alzheimer's disease, multiple system atrophy, striatonigral degeneration, olivopontocerebellar atrophy, and Shy-Drager syndrome.

18. The method of claim 17, wherein the disease is Parkinson's disease.

19. A pharmaceutical composition comprising a compound according to claim 7 and a pharmaceutically acceptable carrier.

20. A method of treating a neurodegenerative disease comprising administering to a mammalian subject in need thereof a pharmaceutically effective amount of a compound according to claim 7 or a composition according to claim 19.

21. The method of claim 20, wherein the disease is an alpha-synucleinopathy.

22. The method of claim 21, wherein the disease is a member selected from the group consisting of Parkinson's disease, Parkinson disease with dementia, PD at risk syndrome, dementia with Lewy bodies, diffuse Lewy body disease, Lewy body dementia, cortical Lewy body disease, senile dementia of Lewy type, Lewy body variant of Alzheimer's disease, diffuse Lewy body type of Alzheimer's disease, combined Parkinson's disease and Alzheimer's disease, multiple system atrophy, striatonigral degeneration, olivopontocerebellar atrophy, and Shy-Drager syndrome.

23. The method of claim 22, wherein the disease is Parkinson's disease.

24. A pharmaceutical composition comprising a compound according to claim 8 and a pharmaceutically acceptable carrier.

25. A method of treating a neurodegenerative disease comprising administering to a mammalian subject in need thereof a pharmaceutically effective amount of a compound according to claim 8 or a composition according to claim 24.

26. The method of claim 25, wherein the disease is an alpha-synucleinopathy.

27. The method of claim 26, wherein the disease is a member selected from the group consisting of Parkinson's disease, Parkinson disease with dementia, PD at risk syndrome, dementia with Lewy bodies, diffuse Lewy body disease, Lewy body dementia, cortical Lewy body disease, senile dementia of Lewy type, Lewy body variant of Alzheimer's disease, diffuse Lewy body type of Alzheimer's disease, combined Parkinson's disease and Alzheimer's disease, multiple system atrophy, striatonigral degeneration, olivopontocerebellar atrophy, and Shy-Drager syndrome.

28. The method of claim 27, wherein the disease is Parkinson's disease.

29. A method of reducing p-Ser-129-alpha-synuclein concentration in brain tissue of a test animal, the method comprising administering to the test animal a compound according to claim 1.

30. A method of reducing p-Ser-129-alpha-synuclein concentration in brain tissue of a test animal, the method comprising administering to the test animal a compound according to claim 6.

31. A method of reducing p-Ser-129-alpha-synuclein concentration in brain tissue of a test animal, the method comprising administering to the test animal a compound according to claim 7.

32. A method of reducing p-Ser-129-alpha-synuclein concentration in brain tissue of a test animal, the method comprising administering to the test animal a compound according to claim 8.

33. A method of treating a cancer comprising administering to a mammalian subject in need thereof a pharmaceutically effective amount of a compound according to claim 1 or a composition according to claim 9.

34. The method of claim 33, wherein the cancer is selected from the group consisting of solid tumors, liquid tumors, tumor metastasis, angiogenic disordors, ocular neovasculization, infantile haemangiomas, acute myelogenous leukemia, chronic myelogenous leukemia, metastatic melanoma, hepatocellular carcinoma, pancreatic carcinoma, brain cancer, non small cell lung cancer, breast cancer, bladder cancer, thyroid cancer, endometrial cancer, prostate cancer, gastric cancer, oropharyngeal cancer, esophageal cancer, head and neck cancer, ovarian carcinomas, papillary carcinomas, colorectal cancers, glioma, glioblastoma, squamous cell carcinoma, hepatoma, melanoma, non-Hodgkins lymphoma, Hodgkin's lymphoma, advanced metastatic cancers, advanced solid tumors, Kaposi's sarcoma, multiple myeloma, and HTLV-1 mediated tumorigenesis.

35. The method of claim 34, wherein the cancer is selected from the group consisting of glioma, glioblastoma, hepatacellular carcinoma, pancreatic carcinoma, colorectal cancer, papillary carcinoma, ovarian carcinoma, non small cell lung cancer, breast cancer, and squamous cell carcinoma.

36. A method of treating a cancer comprising administering to a mammalian subject in need thereof a pharmaceutically effective amount of a compound according to claim 6 or a composition according to claim 14.

37. The method of claim 36, wherein the cancer is selected from the group consisting of solid tumors, liquid tumors, tumor metastasis, angiogenic disordors, ocular neovasculization, infantile haemangiomas, acute myelogenous leukemia, chronic myelogenous leukemia, metastatic melanoma, hepatocellular carcinoma, pancreatic carcinoma, brain cancer, non small cell lung cancer, breast cancer, bladder cancer, thyroid cancer, endometrial cancer, prostate cancer, gastric cancer, oropharyngeal cancer, esophageal cancer, head and neck cancer, ovarian carcinomas, papillary carcinomas, colorectal cancers, glioma, glioblastoma, squamous cell carcinoma, hepatoma, melanoma, non-Hodgkins lymphoma, Hodgkin's lymphoma, advanced metastatic cancers, advanced solid tumors, Kaposi's sarcoma, multiple myeloma, and HTLV-1 mediated tumorigenesis.

38. The method of claim 37, wherein the cancer is selected from the group consisting of glioma, glioblastoma, hepatacellular carcinoma, pancreatic carcinoma, colorectal cancer, papillary carcinoma, ovarian carcinoma, non small cell lung cancer, xzx breast cancer, and squamous cell carcinoma.
